(19) United States
(12) Patent Application Publication
(10) Pub. No.: US 2025/0353862 A1
(43) Pub. Date: Nov. 20, 2025

(54) C-MYC MRNA TRANSLATION MODULATORS AND USES THEREOF IN THE TREATMENT OF CANCER

(71) Applicant: Anima Biotech Inc., Bernardsville, NJ (US)

(72) Inventors: Boaz INBAL, Ramat Gan (IL); Aviad MANDABI, Ramat Gan (IL); Stephen David PENROSE, Ramat Gan (IL); Luba SIMHAEV ISCHAKOV, Ramat Gan (IL); Iris ALROY, Ramat Gan (IL); Rina WASSERMANN, Ramat Gan (IL); Yoni SHEINBERGER, Ramat Gan (IL); Yaode WANG, Beijing (CN); Haitang LI, Beijing (CN); Lothar WILLMS, Hillscheid (DE); Scott Alexander SADLER, Ramat Gan (IL); Shuyu CHU, Ramat Gan (IL)

(73) Assignee: Anima Biotech Inc., Bernardsville, NJ (US)

(21) Appl. No.: 18/868,765

(22) PCT Filed: Jul. 2, 2023
(86) PCT No.: PCT/US2023/026827

§ 371 Date: Nov. 24, 2024

Related U.S. Application Data
(63) Continuation-in-part of application No. 17/856,998, filed on Jul. 3, 2022.
(60) Provisional application No. 63/358,134, filed on Jul. 3, 2022.

(51) Int. Cl. C07D 498/04; A61K 31/429; A61K 31/454; A61K 31/4545; A61K 31/4725; A61K 31/4985; A61K 31/519; A61K 31/5377; A61K 31/5383; A61K 45/06; C07D 471/04; C07D 487/04; C07D 513/04; C07D 513/14

(52) U.S. Cl. CPC C07D 498/04; A61K 31/429; A61K 31/454; A61K 31/4545; A61K 31/4725; A61K 31/4985; A61K 31/519; A61K 31/5377; A61K 31/5383; A61K 45/06; C07D 471/04; C07D 487/04; C07D 513/04; C07D 513/14

(57) ABSTRACT

The present invention relates to novel c-MYC mRNA translation modulators, composition and methods of preparation thereof, and uses thereof in the treatment of cancer.

### FIELD OF THE INVENTION

The present invention relates to novel c-MYC mRNA translation modulators, composition and methods of preparation thereof, and uses thereof in the treatment of cancer.

### BACKGROUND OF THE INVENTION

Cancer is the second most common cause of death in the United States, exceeded only by heart disease. In the United States, cancer accounts for 1 of every 4 deaths. The 5-year relative survival rate for all cancer patients diagnosed in 1996-2003 is 66%, up from 50% in 1975-1977 (Cancer Facts & Figures American Cancer Society: Atlanta, GA (2008)). The rate of new cancer cases decreased by an average 0.6% per year among men between 2000 and 2009 and stayed the same for women. From 2000 through 2009, death rates from all cancers combined decreased on average 1.8% per year among men and 1.4% per year among women. This improvement in survival reflects progress in diagnosing at an earlier stage and improvements in treatment. Discovering highly effective anticancer agents with low toxicity is a primary goal of cancer research.

The Myc family includes three major members, the proto-oncogene c-Myc (cellular Myelocytomatosis, short Myc), as well as L-myc and N-myc. These three Myc homologs are involved in the early stages of carcinogenesis and metastatic spread in most human cancers. In most types of tumors Myc gene is not mutated or duplicated, but its mRNA and/or protein levels are increased, indicating that in cancer Myc overexpression is induced at the level of transcription, mRNA steady state levels and translation. Indeed, myc gene expression normally depends on growth factor signaling and both myc mRNA and Myc protein have very short half-lives (of 30 and 20 min respectively) [Dang, C. V. (2012). MYC on the path to cancer. Cell 149, 22-35]. In tumor cells however, the cellular levels of Myc become independent from such signaling and regulation, and the resulting exacerbated Myc function drives intracellular and extracellular transcription programs that allow tumors to grow and thrive. However, Myc does not necessarily need to be overexpressed in order for a cancer to be highly dependent upon its activity. A study from Soucek et al. (Nature (2008) 455(7213):679-83) shows that tumors that express c-Myc at endogenous levels exhibit tumor regression upon Myc inhibition via a genetically engineered system. Therefore, treatment with a Myc inhibitor is not necessarily limited to cancers that overexpress Myc. Compounds according to this invention may also be used to regulate the translation of Myc mRNA, wherein the direct target for the compounds is a protein or RNA which regulate Myc mRNA translation, and as such any tumor which is Myc dependent will benefit from the therapeutic utility of these compounds.

Due to its extensive pathogenic significance, MYC is an important anticancer target. Deregulated Myc gene is found in a wide range of human hematological malignancies and solid tumors, especially in breast cancer, ovarian carcinoma, acute myeloid leukemia, chronic myelogenous leukemia, Hodgkin's and Burkitt's lymphoma, diffuse large Bcell lymphoma, prostate cancer, colon cancer, gastric cancer, primary central nervous system lymphoma, glioblastoma, medulloblastoma, melanoma, non-small cell lung carcinoma, germinal center-derived lymphomas, esophageal squamous cell carcinoma, osteosarcoma, bladder cancer, pancreatic cancer and lung adenocarcinoma. Recent studies also indicate that deregulation of c-MYC is related to the occurrence of BRAF V600E thyroid cancers, choroid plexus carcinoma, and colitis-associated cancer. In addition, amplification of the MvYC gene was found in a significant number of epithelial ovarian cancer cases. In TCGA datasets, the amplification of Myc occurs in several cancer types, including breast, colorectal, pancreatic, gastric, and uterine cancers.

Although Myc gene is a very important oncogene and considered as a driver in carcinogenesis and MYC protein is a key transcription factor broadly targeting various genes, rational designing a direct Myc inhibitor is still challenging. This is mainly because MYC protein lacks structural regions amenable to therapeutic inhibition by small molecules and is considered an undruggable target [BioDrugs (2019) 33:539-553].

Designing and developing MYC modulators is challenging, primarily because the MYC protein has a disordered structure which lacks a pocket or groove that can act as a binding site for modulators.

Interfering with the MYC transcription, blocking the protein-protein interaction (PPI) of MYC and its cofactors, and influencing on signaling pathways related to MYC were used in the past as potential modulatory targets, but failed to be developed as drug candidates. Myc PPI inhibitors failed to show sufficient efficacy in cell-based assays and animal models due to the requirement of high target occupancy to drive efficacy. Modulators of signaling pathways upstream to myc, for example mTOR modulators, failed due lack of target specificity.

Nevertheless, a therapeutic approach to target c-Myc has remained elusive. The absence of a clear ligand-binding domain establishes a formidable obstacle toward direct inhibition, which is a challenging feature shared among many compelling transcriptional targets in cancer. Thus, alternative modalities that target Myc are required, as outlined herein, namely compounds which regulate Myc mRNA translation.

### SUMMARY OF THE INVENTION

This invention provides a compound or its pharmaceutically acceptable salt, stereoisomer, tautomer, hydrate, N-oxide, reverse amide analog, prodrug, isotopic variants (e.g., deuterated analog), PROTAC, pharmaceutical product or any combination thereof, represented by the structure of formula I and/or I(a)-I(n) and by the structures listed in Table 1, as defined herein below. In various embodiments, the compound is a c-MYC mRNA translation modulator. In various embodiments, the compound is a c-MYC mRNA transcription regulator. In various embodiments, the compound is a c-MYC inhibitor. In various embodiments, the compound is any combination of a c-MYC mRNA translation modulator, c-MYC mRNA transcription regulator and c-MYC inhibitor.

This invention further provides a pharmaceutical composition comprising a compound or its pharmaceutically acceptable salt, stereoisomer, tautomer, hydrate, N-oxide, prodrug, isotopic variants (e.g., deuterated analog), PROTAC, pharmaceutical product or any combination thereof, represented by the structure of formula I and/or I(a)-I(n) and by the structures listed in Table 1, as defined herein below, and a pharmaceutically acceptable carrier.

This invention further provides a method of treating, suppressing, reducing the severity, reducing the risk of developing or inhibiting cancer in a subject, comprising administering a compound represented by the structure of formula I and/or I(a)-I(n) and by the structures listed in Table 1, as defined herein below, to a subject suffering from cancer under conditions effective to treat, suppress, reduce the severity, reduce the risk of developing, or inhibit cancer in said subject.

This invention further provides a method for suppressing, reducing or inhibiting tumor growth in a subject, comprising administering a compound represented by the structure of formula I and/or I(a)-I(n) and by the structures listed in Table 1, as defined herein below, to a subject, under conditions effective to suppress, reduce or inhibit tumor growth in said subject. In some embodiment, the tumor is cancerous. In some embodiment, the subject suffers from cancer.

This invention further provides a method of modulating c-MYC mRNA translation in a cell, comprising contacting a compound represented by the structure of formula I and/or I(a)-I(n) and by the structures listed in Table 1, as defined herein below, with a cell, thereby modulating c-MYC mRNA translation in said cell.

This invention further provides a method of regulating c-MYC mRNA transcription in a cell, comprising contacting a compound represented by the structure of formula I and/or I(a)-I(n) and by the structures listed in Table 1, as defined herein below, with a cell, thereby regulating c-MYC mRNA transcription in said cell.

### DETAILED DESCRIPTION OF THE INVENTION

In various embodiments, this invention is directed to a compound represented by the structure of formula (I):

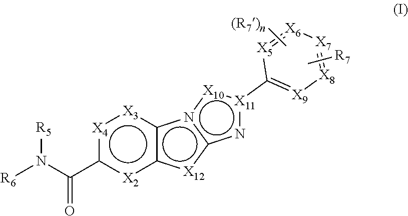

wherein
X₂, X₃, and X₄, are each independently CH, C(R) or N (e.g., C(CH₃), C(O—CH₂-cyclopropyl), C(O—CH₂-methylcyclobutyl), C(NH—CH₂-cyclopropyl), C(isopropoxy), C(O—CH(CH₃)—CH₂—O—CH₃), C(CH₂CH₃), C-iPr, C—CH₂-cyclopropyl, C(OCH₃), C(OCH₂CH₃), C(O—(CH₂)₂—O—CH₃, C(OCHF₂), C(Cl), C(C(O)CH3), C(O—CH₂CH₂—O—CH₃), C(OH));
X₅, X₆, X₇, X₈ and X₉ are each independently nitrogen or carbon atoms;
X₁₀ is N, CH, C(R), or C═O; wherein if X₁₀ is C═O then X₁₁ is N (e.g., C(CH₂—OH), C(CH₂—CH₂—OH), C(NH—CH₂-cyclopropyl), C(COOH), C(CH₃), C(cyclopropyl), C(isopropoxy));
X₁₁ is N or C; wherein if X₁₁ is N then X₁₀ is C═O;
X₁₂ is S, SO₂, O, NH, N(R), N—OH, CH═CH, CH═CH(R), C(R)═CH, N═CH, N═C(R), CH═N or C(R)═N (e.g., N—CH₂—COOH, N—CH₂—CH₂—OH, N—CH₃, N—CH₂CH₃, N-iPr, N-cyclopropyl, N—CH₂-cyclopropyl);
R₅ is H or C₁-C₅ linear or branched alkyl (e.g. methyl);
R₆ is H, F, Cl, Br, I, OH, SH, R₈—OH, R₈—SH, —R₈—O—R₁₀ (e.g., CH₂—O—CH₃, (CH₂)₂O—CH₃ (CH₂)₃O—CH₃, (CH₂)₂O—CH(CH₃)₂), R₈—S—R₁₀ (e.g., (CH₂)₃—S—(CH₂)₂CH₃), R₈—NHC(O)—R₁₀, —O—R₈-R₁₀, R₈-(substituted or unsubstituted C₃-C₈ cycloalkyl) (e.g., CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted, saturated, unsaturated or aromatic, single, fused or spiro 3-10 membered heterocyclic ring) (e.g., (CH₂)₃-piperidine, (CH₂)₃-4-fluoro-piperidine, (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-oxa-azaspirodecane, (CH₂)₃-dimethylpyrazole, CH₂-2-oxo-methylpyrrolidine, CH₂-methyl-azetidine, CH₂-azaspiroheptane), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁) (e.g., (CH₂)₂—NH₂, (CH₂)₃—N(CH₂CH₃)₂, (CH₂)₃—N(CH(CH₃)₂)₂, (CH₂)₃-piperidine, (CH₂)₃-4-fluoro-piperidine, (CH₂)₃-4-cyano-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃)), R₉—R₈—N(R₁₀)(R₁₁) (e.g., (CH₂)₂—C(O)-piperidine), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)CH₃), C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₅ cycloalkyl (e.g., cyclopropyl, cyclobutyl, cyclohexyl, methoxycyclopropyl, methylcyclobutyl, aminomethyl-cyclobutyl, methoxycyclobutyl, 2,3-dihydro-1H-indenol), R₈-(substituted or unsubstituted C₃-C₅ cycloalkyl), substituted or unsubstituted 3-8 membered heterocyclic ring (e.g., piperidine, 1-methyl-piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), substituted or unsubstituted benzyl;
or R₆ and R₅ are joined to form a substituted or unsubstituted 5-8 membered heterocyclic ring (e.g., azepane, piperazine, 2-(piperazin-1-yl)acetamide;
or R₆ is represented by the structure of formula B or Bi:

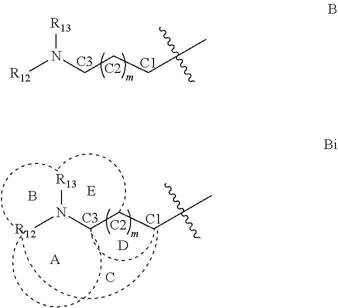

wherein
m is 0 or 1; and
   R₁₂ is R₂₀ or C₁-C₅ C(O)-alkyl, and R₁₃ is R₃₀; or
   R₁₂ and R₁₃ are both H;
   R₁₂ and R₁₃ are each independently H or substituted or unsubstituted C₁-C₅ alkyl (e.g., ethyl, trifluoroethyl);
   R₁₂ and C3 are joined to form ring A and R₁₃ is R₃₀; or
   R₁₂ and R₁₃ are joined to form ring B; or
   R₁₂ and C1 are joined to form ring C and R₁₃ is R₃₀; or
   C1 and C3 are joined to form ring D and R₁₂ and R₁₃ are each independently R₃₀; or
   R₁₃ and C2 are joined to form ring E, m is 1, and R₁₂ is R₃₀; or
   R₁₂ and R₁₃ are joined to form ring B and C1 and C3 are joined to form ring D;
   wherein
      Ring A, C and E are each independently a substituted or unsubstituted single spiro or fused 3-8 membered heterocyclic ring (e.g., A: pyrrolidine, methylpyrrolidine, ethylpyrrolidine); C: piperidine, pyrrolidine, methyl-2-oxopyrrolidine, pyran-pyrrolidine, methyl-azetidine, azabicyclooctane, 2-azabicyclo[2.1.1]hexane, 2-azaspiro[3.3]heptane; E: pyrrolidine, azetidine, ethylpyrrolidine, oxopyrrolidine, methylpiperidine;
      Ring B is a substituted or unsubstituted single, spiro or fused 3-8 membered heterocyclic ring (B: piperidine, piperidin-2-one, 4-fluoropiperidin-2-one, piperidine-4-carbonitrile, 4-fluoropiperidine, 4-fluoro-2-methylpiperidine, methyl-piperidin, fluoropiperidine, difluoropiperidine, pyrrolidine, piperazine, methylpyrrolidine, thiomorpholine 1,1-dioxide, 2-oxa-6-azaspiro[3.3]heptane, methyl-piperazine, dimethyl-pyrazole, imidazole, 2-methyl-2,5-diazabicyclo[2.2.1]heptane, hydroxymethyl-pyrrolidine, diazabicyclo[2.2.1]heptane, 6-fluoro-3-azabicyclo[3.1.1]heptane; and
      Ring D is a substituted or unsubstituted C₃-C₈ cycloalkyl (e.g., cyclobutane, cyclohexane);
R₇ is H, F, Cl, Br, I, OH, O—R₂₀, SH, R₈—OH, R₈—SH, SR₁₀, —R₈—O—R₁₀, —R₈—S—R₁₀, R₈—(C₃-C₅ cycloalkyl), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)), C(O)N(R₁₀)(R₁₁) (e.g., C(O)NH(CH₃), C(O)NH(CH₂CH₂OCH₃), C(O)NH(CH₂CH₂OH)), SO₂R, SO₂N(R₁₀)(R₁₁), CH(CF₃)(NH—R₁₀), C₁-C₅ linear or branched, substituted or unsubstituted alkyl (e.g., methylimidazole, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₅ cyclic alkoxy (e.g. methoxy, ethoxy), optionally wherein at least one methylene group (CH₂) in the alkoxy is replaced with an oxygen atom, C₁-C₅ linear or branched thioalkyl, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopropanol, cyclohexyl), substituted or unsubstituted 4-7 membered heterocyclic ring (e.g., morpholine (e.g., 2 or 3-morpholine), tetrahydrofuran, tetrahydropyran, oxetane, oxetan-3-ol, pyrrolidine, pyrrolidine-3-ol, 1-methylpyrrolidine, pyrrolidin-2-one, pyrrolidinone, imidazole, pyrazole, piperazine, piperidine, piperidine-4-ol, piperidine-4-carbonitrile, 4-fluoropiperidine, oxadiazole, triazole, 2-oxopyrrolidine, pyridine, 1-methylpyridine), R₈-(substituted or unsubstituted single, fused or spiro 3-8 membered heterocyclic ring), substituted or unsubstituted aryl, substituted or unsubstituted benzyl;
or R₇ is represented by the structure of formula A:

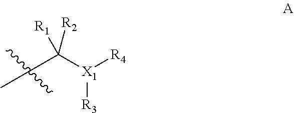

wherein
   X₁ is N or O;
   R₁ and R₂ are each independently H, F, Cl, Br, I, OH, SH, or CF₃, substituted or unsubstituted C₁-C₅ alkyl, C₁-C₅ linear or branched, or C₃-C₈ cyclic haloalkyl, substituted or unsubstituted C₁-C₅ linear or branched, or C₃-C₈ cyclic alkoxy;
   R₁ and R₂ are joined to form ═O or a C₃-C₈ carbocyclic or heterocyclic ring (e.g., cyclopropyl);
   R₃ and R₄ are each independently H, Me, substituted or unsubstituted C₁-C₅ alkyl (e.g., methoxyethylene, methylaminoethyl, aminoethyl), —R₈—O—R₁₀ (e.g., (CH₂)₂O—CH₃), R₈-N(R₁₀)(R₁₁) (e.g., (CH₂)₂—NH(CH₃)), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀;
   R₃ and R₄ are joined to form a 3-8 membered heterocyclic ring (e.g., pyrrolidine, 2-oxopyrrolidine, piperidine, morpholine, piperazine, imidazole);
   or R₂ and R₄ are joined to form a substituted or unsubstituted, saturated or unsaturated, 3-8 membered heterocyclic or carbocyclic ring (e.g., pyrrolidine, 1-methylpyrrolidine, pyrrolidin-2-one, pyridine, piperidine, tetrahydrofurane, tetrahydrothiophene, cyclopropyl, oxetane, imidazole, pyrimidine, triazole, oxadiazole, pyrazole), wherein if the ring is aromatic, then R₁ and/or R₃ are absent;
   wherein if X₁ is O then R₄ is absent;
R₇′ is H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclohexyl), substituted or unsubstituted 3-8 membered heterocyclic ring (e.g., morpholine, pyran, oxetane, pyrrolidine, imidazole, piperazine, piperidine, dioxazole, 2-oxopyrrolidine), substituted or unsubstituted aryl (e.g., phenyl), substituted or unsubstituted benzyl;
or R₇ and R₇′ are joined to form a 5 or 6 membered substituted or unsubstituted, saturated, unsaturated or aromatic, carbocyclic or heterocyclic ring (e.g., piperidine, pyrrolidine, tetrahydrofuran, tetrahydropyran);
R₂₀ is represented by the following structure:

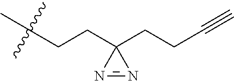 [SMILES: *CCC1(CCC#C)N=N1]

R₃₀ is H, R₂₀, F, Cl, Br, I, OH, SH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);
R is H, F, Cl, Br, I, OH, SH, COOH, CO(R₁₀) (e.g., C(O)CH₃), NH(R₁₀), NH—CH₂-cyclopropyl, N(R₁₀)(R₁₁), CF₃, CN, NO₂, C₁-C₅ linear or branched, substituted or unsubstituted alkyl (e.g., methyl, ethyl, iPr, CH₂-cyclopropyl, CH₂—OH, CH₂—CH₂—OH, CH₂—CH₂—O—CH₂—CH₂—O—CH₃, CH₂—O—CH₂—CH₂-O—CH₃), C₃-C₈ substituted or unsubstituted cycloalkyl, cyclopropyl, substituted or unsubstituted C₁-C₅ linear or branched alkoxy, (e.g., methoxy, ethoxy, O—(CH₂CH₃), OCHF₂, O—(CH₂)₂O—CH₃, isopropoxy, O—(CH₂)-cyclopropyl, O—CH₂-methylcyclobutyl, O—CH₂-3-methyloxetane, O—CH(CH₃)—CH₂—O—CH₃) C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, O—R₈-R₁₀ (e.g. O—(CH₂)₂O—CH₃, O—(CH₂CH₃), O—(CH₂)-cyclopropyl), —R₈—R₁₀ (e.g., (CH₂)-cyclopropyl, (CH₂)₂O—CH₃, (CH₂)—OH, (CH₂)₂—OH, (CH₂)—COOH), substituted or unsubstituted aryl (e.g., phenyl), substituted or unsubstituted heteroaryl (e.g., pyridine (2, 3, and 4-pyridine);
each R₈ is independently [CH₂]ₚ
   wherein p is between 1 and 10 (e.g., 1, 2);
R₉ is [CH]_{q}, [C]_{q}
   wherein q is between 2 and 10;
R₁₀ and R₁₁ are each independently H, OH, COOH, C₁-C₅ substituted or unsubstituted linear or branched alkyl (e.g., methyl, ethyl, CH₂-cyclopropyl, CH₂—CH₂—O—CH₃), C₃-C₈ substituted or unsubstituted cycloalkyl (e.g., cyclopropyl), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;
or R₁₀ and R₁₁ are joined to form a substituted or unsubstituted 3-8 membered heterocyclic ring (e.g., piperazine, piperidine),
n is an integer between 0 and 4 (e.g., 1, 2);
or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In some embodiments, at least one of X₂, X₃, and X₄ is C(R). In some embodiments, X₁₁ is N. In some embodiments, X₁₂ is not S. In some embodiments, at least one of X₂, X₃, and X₄ is C(R); X₁₁ is N; or X₁₂ is not S. In some embodiments, R is not H.

In various embodiments, this invention is directed to a compound represented by the structure of formula I(a):

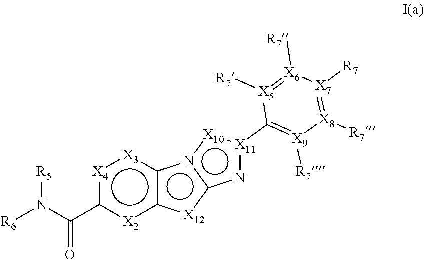

wherein
X₂, X₃, and X₄, are each independently CH, C(R) or N (e.g., C(CH₃), C(O—CH₂-cyclopropyl), C(O—CH₂-methylcyclobutyl), C(NH—CH₂-cyclopropyl), C(isopropoxy), C(O—CH(CH₃)—CH₂—O—CH₃), C(CH₂CH₃), C-iPr, C—CH₂-cyclopropyl, C(OCH₃), C(OCH₂CH₃), C(O—(CH₂)₂—O—CH₃, C(OCHF₂), C(Cl), C(C(O)CH3), C(O—CH₂CH₂—O—CH₃), C(OH));
X₅, X₆, X₇, X₈ and X₉ are each independently nitrogen or carbon atoms;
   wherein if either one of X₅, X₆, X₇, X₈ and X₉ is nitrogen, then the respective R₇′, R₇″, R₇, R₇′″, and R₇″″ substitution is absent;
X₁₀ is N, CH, C(R), or C═O; wherein if X₁₀ is C═O then X₁₁ is N (e.g., C(CH₂—OH), C(CH₂—CH₂—OH), C(NH—CH₂-cyclopropyl), C(COOH), C(CH₃), C(cyclopropyl), C(isopropoxy));
X₁₁ is N or C; wherein if X₁₁ is N then X₁₀ is C═O;
X₁₂ is S, SO₂, O, NH, N(R), N—OH, CH═CH, CH═CH(R), C(R)═CH, N═CH, N═C(R), CH═N or C(R)═N (e.g., N—CH₂—COOH, N—CH₂—CH₂—OH, N—CH₃, N—CH₂CH₃, N-iPr, N-cyclopropyl, N—CH₂-cyclopropyl);
R₅ is H or C₁-C₅ linear or branched alkyl (e.g. methyl);
R₆ is H, F, Cl, Br, I, OH, SH, R₈—OH, R₈—SH, —R₈—O—R₁₀ (e.g., CH₂—O—CH₃, (CH₂)₂O—CH₃ (CH₂)₃O—CH₃, (CH₂)₂O—CH(CH₃)₂), R₈—S—R₁₀ (e.g., (CH₂)₃—S—(CH₂)₂CH₃), R₈—NHC(O)—R₁₀, —O—R₈—R₁₀, R₈-(substituted or unsubstituted C₃-C₈ cycloalkyl) (e.g., CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted, saturated, unsaturated or aromatic, single, fused or spiro 3-10 membered heterocyclic ring) (e.g., (CH₂)₃-piperidine, (CH₂)₃-4-fluoro-piperidine, (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-oxa-azaspirodecane, (CH₂)₃-dimethylpyrazole, CH₂-2-oxo-methylpyrrolidine, CH₂-methyl-azetidine, CH₂-azaspiroheptane), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁) (e.g., (CH₂)₂—NH₂, (CH₂)₃—N(CH₂CH₃)₂, (CH₂)₃—N(CH(CH₃)₂)₂, (CH₂)₃-piperidine, (CH₂)₃-4-fluoro-piperidine, (CH₂)₃-4-cyano-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃)), R₉—R₈—N(R₁₀)(R₁₁) (e.g., (CH₂)₂—C(O)-piperidine), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)CH₃), C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₅ cycloalkyl (e.g., cyclopropyl, cyclobutyl, cyclohexyl, methoxycyclopropyl, methylcyclobutyl, aminomethyl-cyclobutyl, methoxycyclobutyl, 2,3-dihydro-1H-indenol), R₈-(substituted or unsubstituted C₃-C₈ cycloalkyl), substituted or unsubstituted 3-8 membered heterocyclic ring (e.g., piperidine, 1-methyl-piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), substituted or unsubstituted benzyl;
or R₆ and R₅ are joined to for a substituted or unsubstituted 5-8 membered heterocyclic ring (e.g., azepane, piperazine, 2-(piperazin-1-yl)acetamide;
or R₆ is represented by the structure of formula B or Bi:

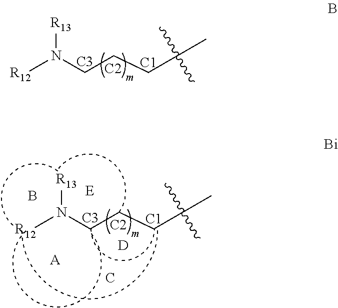

wherein
m is 0 or 1; and
   R₁₂ is R₂₀ or C₁-C₅ C(O)-alkyl, and R₁₃ is R₃₀; or
   R₁₂ and R₁₃ are both H;
   R₁₂ and R₁₃ are each independently H or substituted or unsubstituted C₁-C₅ alkyl (e.g., ethyl, trifluoroethyl);
   R₁₂ and C3 are joined to form ring A and R₁₃ is R₃₀; or
   R₁₂ and R₁₃ are joined to form ring B; or
   R₁₂ and C1 are joined to form ring C and R₁₃ is R₃₀; or
   C1 and C3 are joined to form ring D and R₁₂ and R₁₃ are each independently R₃₀; or
   R₁₃ and C2 are joined to form ring E, m is 1, and R₁₂ is R₃₀; or
   R₁₂ and R₁₃ are joined to form ring B and C1 and C3 are joined to form ring D;
   wherein
      Ring A, C and E are each independently a substituted or unsubstituted single spiro or fused 3-8 membered heterocyclic ring (e.g., A: pyrrolidine, methylpyrrolidine, ethylpyrrolidine); C: piperidine, pyrrolidine, methyl-2-oxopyrrolidine, pyran-pyrrolidine, methyl-azetidine, azabicyclooctane, 2-azabicyclo[2.1.1]hexane, 2-azaspiro[3.3]heptane; E: pyrrolidine, azetidine, ethylpyrrolidine, oxopyrrolidine, methylpiperidine);
      Ring B is a substituted or unsubstituted single, spiro or fused 3-8 membered heterocyclic ring (B: piperidine, piperidin-2-one, 4-fluoropiperidin-2-one, piperidine-4-carbonitrile, 4-fluoropiperidine, 4-fluoro-2-methylpiperidine, methyl-piperidin, fluoropiperidine, difluoropiperidine, pyrrolidine, piperazine, methylpyrrolidine, thiomorpholine 1,1-dioxide, 2-oxa-6-azaspiro[3.3]heptane, methyl-piperazine, dimethyl-pyrazole, imidazole, 2-methyl-2,5-diazabicyclo[2.2.1]heptane, hydroxymethyl-pyrrolidine, diazabicyclo[2.2.1]heptane, 6-fluoro-3-azabicyclo[3.1.1]heptane; and
      Ring D is a substituted or unsubstituted C₃-C₈ cycloalkyl (e.g., cyclobutane, cyclohexane);
R₇ is H, F, Cl, Br, I, OH, O—R₂₀, SH, R₈—OH, R₈—SH, SR₁₀, —R₈—O—R₁₀, —R₈—S—R₁₀, R₈—(C₃-C₅ cycloalkyl), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)), C(O)N(R₁₀)(R₁₁) (e.g., C(O)NH(CH₃), C(O)NH(CH₂CH₂OCH₃), C(O)NH(CH₂CH₂OH)), SO₂R, SO₂N(R₁₀)(R₁₁), CH(CF₃)(NH—R₁₀), C₁-C₅ linear or branched, substituted or unsubstituted alkyl (e.g., methylimidazole, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₅ cyclic alkoxy (e.g. methoxy, ethoxy), optionally wherein at least one methylene group (CH₂) in the alkoxy is replaced with an oxygen atom, C₁-C₅ linear or branched thioalkyl, C₁-C₅ linear or branched thioalkoxy, C₁-C₅ linear or branched haloalkoxy, C₁-C₈ linear or branched alkoxyalkyl, substituted or unsubstituted C₃-C₈ cycloalkyl (e.g., cyclopropyl, cyclopropanol, cyclohexyl), substituted or unsubstituted 4-7 membered heterocyclic ring (e.g., morpholine (e.g., 2 or 3-morpholine), tetrahydrofuran, tetrahydropyran, oxetane, oxetan-3-ol, pyrrolidine, pyrrolidine-3-ol, 1-methylpyrrolidine, pyrrolidin-2-one, pyrrolidinone, imidazole, pyrazole, piperazine, piperidine, piperidine-4-ol, piperidine-4-carbonitrile, 4-fluoropiperidine, oxadiazole, triazole, 2-oxopyrrolidine, pyridine, 1-methylpyridine), R₈-(substituted or unsubstituted single, fused or spiro 3-8 membered heterocyclic ring), substituted or unsubstituted aryl, substituted or unsubstituted benzyl;
or R₇ is represented by the structure of formula A:

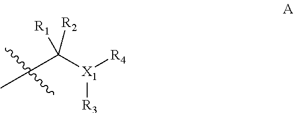

wherein
   X₁ is N or O;
   R₁ and R₂ are each independently H, F, Cl, Br, I, OH, SH, or CF₃, substituted or unsubstituted C₁-C₅ alkyl, C₁-C₅ linear or branched, or C₃-C₈ cyclic haloalkyl, substituted or unsubstituted C₁-C₅ linear or branched, or C₃-C₈ cyclic alkoxy;
   R₁ and R₂ are joined to form ═O or a C₃-C₈ carbocyclic or heterocyclic ring (e.g., cyclopropyl);
   R₃ and R₄ are each independently H, Me, substituted or unsubstituted C₁-C₅ alkyl (e.g., methoxyethylene, methylaminoethyl, aminoethyl), —R₈—O—R₁₀ (e.g., (CH₂)₂O—CH₃), R₈—N(R₁₀)(R₁₁) (e.g., (CH₂)₂—NH(CH₃)), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀;
   R₃ and R₄ are joined to form a 3-8 membered heterocyclic ring (e.g., pyrrolidine, 2-oxopyrrolidine, piperidine, morpholine, piperazine, imidazole);
   or R₂ and R₄ are joined to form a substituted or unsubstituted, saturated or unsaturated, 3-8 membered heterocyclic or carbocyclic ring (e.g., pyrrolidine, 1-methylpyrrolidine, pyrrolidin-2-one, pyridine, piperidine, tetrahydrofurane, tetrahydrothiophene, cyclopropyl, oxetane, imidazole, pyrimidine, triazole, oxadiazole, pyrazole), wherein if the ring is aromatic, then R₁ and/or R₃ are absent;
   wherein if X₁ is O then R₄ is absent;
R₇′, R₇″, R₇′″ and R₇″″ are each independently H, F, Cl, Br, I, OH, O—R₂₀, SH, R₈—OH, R₈—SH, —R₈—O—R₁₀, R₈—(C₃-C₅ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclohexyl), substituted or unsubstituted 3-8 membered heterocyclic ring (e.g., morpholine, pyran, oxetane, pyrrolidine, tetrahydrofuran, imidazole, piperazine, piperidine, dioxazole, 2-oxopyrrolidine), substituted or unsubstituted aryl (e.g., phenyl), substituted or unsubstituted benzyl;
or R₇′ and R₇″ are joined to form a 3-8 membered substituted or unsubstituted, saturated, unsaturated or aromatic, carbocyclic or heterocyclic ring (e.g., cyclopentyl, cyclohexyl, piperidine, tetrahydrofuran, tetrahydropyran, pyrrolidine);
or R₇″ and R₇ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
or R₇ and R₇′″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
or R₇′″ and R₇″″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
R₂₀ is represented by the following structure:

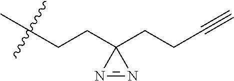 [SMILES: *CCC1(CCC#C)N=N1]

R₃₀ is H, R₂₀, F, Cl, Br, I, OH, SH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);
R is H, F, Cl, Br, I, OH, SH, COOH, CO(R₁₀) (e.g., C(O)CH₃), NH(R₁₀), NH—CH₂-cyclopropyl, N(R₁₀)(R₁₁), CF₃, CN, NO₂, C₁-C₅ linear or branched, substituted or unsubstituted alkyl (e.g., methyl, ethyl, iPr, CH₂-cyclopropyl, CH₂—OH, CH₂—CH₂—OH, CH₂—CH₂—O—CH₂—CH₂—O—CH₃, CH₂—O—CH₂—CH₂-O—CH₃), C₃-C₈ substituted or unsubstituted cycloalkyl, cyclopropyl, substituted or unsubstituted C₁-C₅ linear or branched alkoxy, (e.g., methoxy, ethoxy, O—(CH₂CH₃), OCHF₂, O—(CH₂)₂O—CH₃, isopropoxy, O—(CH₂)-cyclopropyl, O—CH₂-methylcyclobutyl, O—CH₂-3-methyloxetane, O—CH(CH₃)—CH₂—O—CH₃) C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, O—R₈-R₁₀ (e.g. O—(CH₂)₂O—CH₃, O—(CH₂CH₃), O—(CH₂)-cyclopropyl), —R₈—R₁₀ (e.g., (CH₂)-cyclopropyl, (CH₂)₂O—CH₃, (CH₂)—OH, (CH₂)₂—OH, (CH₂)—COOH), substituted or unsubstituted aryl (e.g., phenyl), substituted or unsubstituted heteroaryl (e.g., pyridine (2, 3, and 4-pyridine);
each R₈ is independently [CH₂]ₚ
   wherein p is between 1 and 10 (e.g., 1, 2);
R₉ is [CH]_{q}, [C]_{q}
   wherein q is between 2 and 10;
R₁₀ and R₁₁ are each independently H, OH, COOH, C₁-C₅ substituted or unsubstituted linear or branched alkyl (e.g., methyl, ethyl, CH₂-cyclopropyl, CH₂—CH₂—O—CH₃), C₃-C₈ substituted or unsubstituted cycloalkyl (e.g., cyclopropyl), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;
or R₁₀ and R₁₁ are joined to form a substituted or unsubstituted 3-8 membered heterocyclic ring (e.g., piperazine, piperidine),
n is an integer between 0 and 4 (e.g., 1, 2);
or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In some embodiments, at least one of R₇, R₇′, R₇″, R₇′″ and R₇″″ is not H. In some embodiments, at least two of R₇, R₇′, R₇″, R₇′″ and R₇″″ are not H. In some embodiments, at least one of R₇′, R₇″, R₇′″ and R₇″″ is not H. In some embodiments, at least two of R₇′, R₇″, R₇′″ and R₇″″ are not H. In some embodiments, at least two of R₇′, R₇″, R₇′″ and R₇″″ are different than each other. In some embodiments, at least two of R₇′, R₇″, R₇′″ and R₇″″ are not H and are different than each other.

In some embodiments, at least one of X₂, X₃, and X₄ is C(R). In some embodiments, X₁₁ is N. In some embodiments, X₁₂ is not S. In some embodiments, at least one of X₂, X₃, and X₄ is C(R); X₁₁ is N; or X₁₂ is not S. In some embodiments, R is not H.

In various embodiments, this invention is directed to a compound represented by the structure of formula I(b):

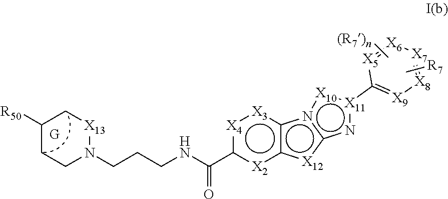

wherein
X₂, X₃, and X₄, are each independently CH, C(R) or N (e.g., C(CH₃), C(O—CH₂-cyclopropyl), C(O—CH₂-methylcyclobutyl), C(NH—CH₂-cyclopropyl), C(isopropoxy), C(O—CH(CH₃)—CH₂—O—CH₃), C(CH₂CH₃), C-iPr, C—CH₂-cyclopropyl, C(OCH₃), C(OCH₂CH₃), C(O—(CH₂)₂—O—CH₃, C(OCHF₂), C(Cl), C(C(O)CH3), C(O—CH₂CH₂—O—CH₃), C(OH));
X₅, X₆, X₇, X₈ and X₉ are each independently nitrogen or carbon atoms;
X₁₀ is N, CH, C(R), or C═O; wherein if X₁₀ is C═O then X₁₁ is N (e.g., C(CH₂—OH), C(CH₂—CH₂—OH), C(NH—CH₂-cyclopropyl), C(COOH), C(CH₃), C(cyclopropyl), C(isopropoxy));
X₁₁ is N or C; wherein if X₁₁ is N then X₁₀ is C═O;
X₁₂ is S, SO₂, O, NH, N(R), N—OH, CH═CH, CH═CH(R), C(R)═CH, N═CH, N═C(R), CH═N or C(R)═N (e.g., N—CH₂—COOH, N—CH₂—CH₂—OH, N—CH₃, N—CH₂CH₃, N-iPr, N-cyclopropyl, N—CH₂-cyclopropyl);
X₁₃ is CH₂, CH(R) (e.g., CH—CH₃), C(R)₂, or C═O;
Ring G is absent or is a substituted or unsubstituted 3-8 membered carbocyclic or heterocyclic ring (e.g., cyclobutane, cyclopentane, cyclohexane);
R₇ is H, F, Cl, Br, I, OH, O—R₂₀, SH, R₈—OH, R₈—SH, SR₁₀, —R₈—O—R₁₀, —R₈—S—R₁₀, R₈—(C₃-C₈ cycloalkyl), CF₃, CD₃, OCD₃, CN, NO₂, —CH₂CN, —R₈CN, NH₂, NHR, N(R)₂, NH(R₁₀), N(R₁₀)(R₁₁), R₈-N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)), C(O)N(R₁₀)(R₁₁) (e.g., C(O)NH(CH₃), C(O)NH(CH₂CH₂OCH₃), C(O)NH(CH₂CH₂OH)), SO₂R, SO₂N(R₁₀)(R₁₁), CH(CF₃)(NH—R₁₀), C₁-C₅ linear or branched, substituted or unsubstituted alkyl (e.g., methylimidazole, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy, ethoxy), optionally wherein at least one methylene group (CH₂) in the alkoxy is replaced with an oxygen atom, C₁-C₅ linear or branched thioalkyl, C₁-C₅ linear or branched thioalkoxy, C₁-C₅ linear or branched haloalkoxy, C₁-C₈ linear or branched alkoxyalkyl, substituted or unsubstituted C₃-C₈ cycloalkyl (e.g., cyclopropyl, cyclopropanol, cyclohexyl), substituted or unsubstituted 4-7 membered heterocyclic ring (e.g., morpholine (e.g., 2 or 3-morpholine), tetrahydrofuran, tetrahydropyran, oxetane, oxetan-3-ol, pyrrolidine, pyrrolidine-3-ol, 1-methylpyrrolidine, pyrrolidin-2-one, pyrrolidinone, imidazole, pyrazole, piperazine, piperidine, piperidine-4-ol, piperidine-4-carbonitrile, 4-fluoropiperidine, oxadiazole, triazole, 2-oxopyrrolidine, pyridine, 1-methylpyridine), R₈-(substituted or unsubstituted single, fused or spiro 3-8 membered heterocyclic ring), substituted or unsubstituted aryl, substituted or unsubstituted benzyl;
or R₇ is represented by the structure of formula A:

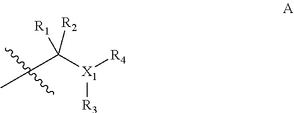

wherein
   X₁ is N or O;
   R₁ and R₂ are each independently H, F, Cl, Br, I, OH, SH, or CF₃, substituted or unsubstituted C₁-C₅ alkyl, C₁-C₅ linear or branched, or C₃-C₈ cyclic haloalkyl, substituted or unsubstituted C₁-C₅ linear or branched, or C₃-C₈ cyclic alkoxy;
   R₁ and R₂ are joined to form ═O or a C₃-C₈ carbocyclic or heterocyclic ring (e.g., cyclopropyl);
   R₃ and R₄ are each independently H, Me, substituted or unsubstituted C₁-C₅ alkyl (e.g., methoxyethylene, methylaminoethyl, aminoethyl), —R₈—O—R₁₀ (e.g., (CH₂)₂O—CH₃), R₈—N(R₁₀)(R₁₁) (e.g., (CH₂)₂—NH(CH₃)), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀;
   R₃ and R₄ are joined to form a 3-8 membered heterocyclic ring (e.g., pyrrolidine, 2-oxopyrrolidine, piperidine, morpholine, piperazine, imidazole);
   or R₂ and R₄ are joined to form a substituted or unsubstituted, saturated or unsaturated, 3-8 membered heterocyclic or carbocyclic ring (e.g., pyrrolidine, 1-methylpyrrolidine, pyrrolidin-2-one, pyridine, piperidine, tetrahydrofurane, tetrahydrothiophene, cyclopropyl, oxetane, imidazole, pyrimidine, triazole, oxadiazole, pyrazole), wherein if the ring is aromatic, then R₁ and/or R₃ are absent;
   wherein if X₁ is O then R₄ is absent;
R₇′ is H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclohexyl), substituted or unsubstituted 3-8 membered heterocyclic ring (e.g., morpholine, pyran, oxetane, pyrrolidine, imidazole, piperazine, piperidine, dioxazole, 2-oxopyrrolidine), substituted or unsubstituted aryl (e.g., phenyl), substituted or unsubstituted benzyl;
or R₇ and R₇′ are joined to form a 5 or 6 membered substituted or unsubstituted, saturated, unsaturated or aromatic, carbocyclic or heterocyclic ring (e.g., piperidine, pyrrolidine, tetrahydrofuran, tetrahydropyran);
R₂₀ is represented by the following structure:

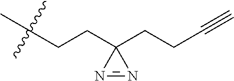 [SMILES: *CCC1(CCC#C)N=N1]

R₃₀ is H, R₂₀, F, Cl, Br, I, OH, SH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);
R₅₀ is H, R₂₀, F, Cl, Br, I, OH, SH, N(R)₂, NH(R₁₀), N(R₁₀)(R₁₁), CF₃, CN, NO₂, C₁-C₅ linear or branched, substituted or unsubstituted alkyl C₁-C₅ linear or branched alkoxy, C₁-C₅ linear or branched haloalkyl, —R₈-R₁₀ (e.g., (CH₂)₂O—CH₃), substituted or unsubstituted aryl (e.g., phenyl), substituted or unsubstituted heteroaryl (e.g., pyridine (2, 3, and 4-pyridine);
R is H, F, Cl, Br, I, OH, SH, COOH, CO(R₁₀) (e.g., C(O)CH₃), NH(R₁₀), NH—CH₂-cyclopropyl, N(R₁₀)(R₁₁), CF₃, CN, NO₂, C₁-C₅ linear or branched, substituted or unsubstituted alkyl (e.g., methyl, ethyl, iPr, CH₂-cyclopropyl, CH₂—OH, CH₂—CH₂—OH, CH₂—CH₂—O—CH₂—CH₂—O—CH₃, CH₂—O—CH₂—CH₂-O—CH₃), C₃-C₈ substituted or unsubstituted cycloalkyl, cyclopropyl, substituted or unsubstituted C₁-C₅ linear or branched alkoxy, (e.g., methoxy, ethoxy, O—(CH₂CH₃), OCHF₂, O—(CH₂)₂O—CH₃, isopropoxy, O—(CH₂)-cyclopropyl, O—CH₂-methylcyclobutyl, O—CH₂-3-methyloxetane, O—CH(CH₃)—CH₂—O—CH₃) C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, O—R₈-R₁₀ (e.g. O—(CH₂)₂O—CH₃, O—(CH₂CH₃), O—(CH₂)-cyclopropyl), —R₈-R₁₀ (e.g., (CH₂)-cyclopropyl, (CH₂)₂O—CH₃, (CH₂)—OH, (CH₂)₂—OH, (CH₂)—COOH), substituted or unsubstituted aryl (e.g., phenyl), substituted or unsubstituted heteroaryl (e.g., pyridine (2, 3, and 4-pyridine);
each R₅ is independently [CH₂]p
   wherein p is between 1 and 10 (e.g., 1, 2);
R₉ is [CH]_{q}, [C]_{q}
   wherein q is between 2 and 10;
R₁₀ and R₁₁ are each independently H, OH, COOH, C₁-C₅ substituted or unsubstituted linear or branched alkyl (e.g., methyl, ethyl, CH₂-cyclopropyl, CH₂—CH₂—O—CH₃), C₃-C₈ substituted or unsubstituted cycloalkyl (e.g., cyclopropyl), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;
or R₁₀ and R₁₁ are joined to form a substituted or unsubstituted 3-8 membered heterocyclic ring (e.g., piperazine, piperidine),
n is an integer between 0 and 4 (e.g., 1, 2);
or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In some embodiments, at least one of X₂, X₃, and X₄ is C(R). In some embodiments, X₁₁ is N. In some embodiments, X₁₂ is not S. In some embodiments, at least one of X₂, X₃, and X₄ is C(R); X₁₁ is N; or X₁₂ is not S. In some embodiments, R is not H.

In various embodiments, this invention is directed to a compound represented by the structure of formula I(c):

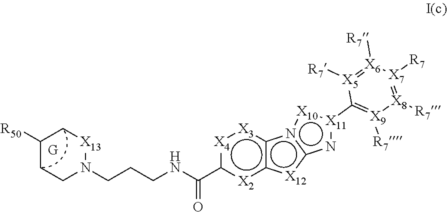

wherein
X₂, X₃, and X₄, are each independently CH, C(R) or N (e.g., C(CH₃), C(O—CH₂-cyclopropyl), C(O—CH₂-methylcyclobutyl), C(NH—CH₂-cyclopropyl), C(isopropoxy), CO—CH(CH₃)—CH₂—O—CH₃), C(CH₂CH₃), C-iPr, C—CH₂-cyclopropyl, C(OCH₃), C(OCH₂CH₃), C(O—(CH₂)₂O—CH₃, C(OCHF₂), C(Cl), C(C(O)CH3), C(O—CH₂CH₂—O—CH₃), C(OH));
X₅, X₆, X₇, X₈ and X₉ are each independently nitrogen or carbon atoms;
   wherein if either one of X₅, X₆, X₇, X₈ and X₉ is nitrogen, then the respective R₇′, R₇″, R₇, R₇′″, and R₇″″ substitution is absent;
X₁₀ is N, CH, C(R), or C═O; wherein if X₁₀ is C═O then X₁₁ is N (e.g., C(CH₂—OH), C(CH₂—CH₂—OH), C(NH—CH₂-cyclopropyl), C(COOH), C(CH₃), C(cyclopropyl), C(isopropoxy));
X₁₁ is N or C; wherein if X₁₁ is N then X₁₀ is C═O;
X₁₂ is S, SO₂, O, NH, N(R), N—OH, CH═CH, CH═CH(R), C(R)═CH, N═CH, N═C(R), CH═N or C(R)═N (e.g., N—CH₂—COOH, N—CH₂—CH₂—OH, N—CH₃, N—CH₂CH₃, N-iPr, N-cyclopropyl, N—CH₂-cyclopropyl);
X₁₃ is CH₂, CH(R) (e.g., CH—CH₃), C(R)₂, or C═O;
Ring G is absent or is a substituted or unsubstituted 3-8 membered carbocyclic or heterocyclic ring (e.g., cyclobutane, cyclopentane, cyclohexane);
R₇ is H, F, Cl, Br, I, OH, O—R₂₀, SH, R₈—OH, R₈—SH, SR₁₀, —R₈—O—R₁₀, —R₈—S—R₁₀, R₈—(C₃-C₅ cycloalkyl), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)), C(O)N(R₁₀)(R₁₁) (e.g., C(O)NH(CH₃), C(O)NH(CH₂CH₂OCH₃), C(O)NH(CH₂CH₂OH)), SO₂R, SO₂N(R₁₀)(R₁₁), CH(CF₃)(NH—R₁₀), C₁-C₈ linear or branched, substituted or unsubstituted alkyl (e.g., methylimidazole, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy, ethoxy), optionally wherein at least one methylene group (CH₂) in the alkoxy is replaced with an oxygen atom, C₁-C₅ linear or branched thioalkyl, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopropanol, cyclohexyl), substituted or unsubstituted 4-7 membered heterocyclic ring (e.g., morpholine (e.g., 2 or 3-morpholine), tetrahydrofuran, tetrahydropyran, oxetane, oxetan-3-ol, pyrrolidine, pyrrolidine-3-ol, 1-methylpyrrolidine, pyrrolidin-2-one, pyrrolidinone, imidazole, pyrazole, piperazine, piperidine, piperidine-4-ol, piperidine-4-carbonitrile, 4-fluoropiperidine, oxadiazole, triazole, 2-oxopyrrolidine, pyridine, 1-methylpyridine), R₈-(substituted or unsubstituted single, fused or spiro 3-8 membered heterocyclic ring), substituted or unsubstituted aryl, substituted or unsubstituted benzyl;
or R₇ is represented by the structure of formula A:

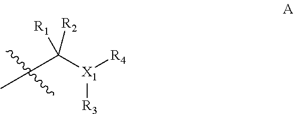

wherein
   X₁ is N or O;
   R₁ and R₂ are each independently H, F, Cl, Br, I, OH, SH, or CF₃, substituted or unsubstituted C₁-C₅ alkyl, C₁-C₅ linear or branched, or C₃-C₈ cyclic haloalkyl, substituted or unsubstituted C₁-C₅ linear or branched, or C₃-C₈ cyclic alkoxy;
   R₁ and R₂ are joined to form ═O or a C₃-C₈ carbocyclic or heterocyclic ring (e.g., cyclopropyl);
   R₃ and R₄ are each independently H, Me, substituted or unsubstituted C₁-C₅ alkyl (e.g., methoxyethylene, methylaminoethyl, aminoethyl), —R₈—O—R₁₀ (e.g., (CH₂)₂O—CH₃), R₈—N(R₁₀)(R₁₁) (e.g., (CH₂)₂—NH(CH₃)), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀;
   R₃ and R₄ are joined to form a 3-8 membered heterocyclic ring (e.g., pyrrolidine, 2-oxopyrrolidine, piperidine, morpholine, piperazine, imidazole);
   or R₂ and R₄ are joined to form a substituted or unsubstituted, saturated or unsaturated, 3-8 membered heterocyclic or carbocyclic ring (e.g., pyrrolidine, 1-methylpyrrolidine, pyrrolidin-2-one, pyridine, piperidine, tetrahydrofurane, tetrahydrothiophene, cyclopropyl, oxetane, imidazole, pyrimidine, triazole, oxadiazole, pyrazole), wherein if the ring is aromatic, then R₁ and/or R₃ are absent;
   wherein if X₁ is O then R₄ is absent;
R₇′, R₇″, R₇′″ and R₇″″ are each independently H, F, Cl, Br, I, OH, O—R₂₀, SH, R₈—OH, R₈—SH, —R₈—O—R₁₀, R₈—(C₃-C₅ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclohexyl), substituted or unsubstituted 3-8 membered heterocyclic ring (e.g., morpholine, pyran, oxetane, pyrrolidine, tetrahydrofuran, imidazole, piperazine, piperidine, dioxazole, 2-oxopyrrolidine), substituted or unsubstituted aryl (e.g., phenyl), substituted or unsubstituted benzyl;
or R₇′ and R₇″ are joined to form a 3-8 membered substituted or unsubstituted, saturated, unsaturated or aromatic, carbocyclic or heterocyclic ring (e.g., cyclopentyl, cyclohexyl, piperidine, tetrahydrofuran, tetrahydropyran, pyrrolidine);
or R₇″ and R₇ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
or R₇ and R₇′″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
or R₇′″ and R₇″″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
R₂₀ is represented by the following structure:

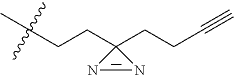 [SMILES: *CCC1(CCC#C)N=N1]

R₃₀ is H, R₂₀, F, Cl, Br, I, OH, SH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);
R₅₀ is H, R₂₀, F, Cl, Br, I, OH, SH, N(R)₂, NH(R₁₀), N(R₁₀)(R₁₁), CF₃, CN, NO₂, C₁-C₅ linear or branched, substituted or unsubstituted alkyl C₁-C₅ linear or branched alkoxy, C₁-C₅ linear or branched haloalkyl, —R₈—R₁₀ (e.g., (CH₂)₂O—CH₃), substituted or unsubstituted aryl (e.g., phenyl), substituted or unsubstituted heteroaryl (e.g., pyridine (2, 3, and 4-pyridine);
R is H, F, Cl, Br, I, OH, SH, COOH, CO(R₁₀) (e.g., C(O)CH₃), NH(R₁₀), NH—CH₂-cyclopropyl, N(R₁₀)(R₁₁), CF₃, CN, NO₂, C₁-C₅ linear or branched, substituted or unsubstituted alkyl (e.g., methyl, ethyl, iPr, CH₂-cyclopropyl, CH₂—OH, CH₂—CH₂—OH, CH₂—CH₂—O—CH₂—CH₂—O—CH₃, CH₂—O—CH₂—CH₂-O—CH₃), C₃-C₈ substituted or unsubstituted cycloalkyl, cyclopropyl, substituted or unsubstituted C₁-C₅ linear or branched alkoxy, (e.g., methoxy, ethoxy, O—(CH₂CH₃), OCHF₂, O—(CH₂)₂O—CH₃, isopropoxy, O—(CH₂)-cyclopropyl, O—CH₂-methylcyclobutyl, O—CH₂-3-methyloxetane, O—CH(CH₃)—CH₂—O—CH₃) C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, O—R₈-R₁₀ (e.g. O—(CH₂)₂O—CH₃, O—(CH₂CH₃), O—(CH₂)-cyclopropyl), —R₈—R₁₀ (e.g., (CH₂)-cyclopropyl, (CH₂)₂O—CH₃, (CH₂)—OH, (CH₂)₂—OH, (CH₂)—COOH), substituted or unsubstituted aryl (e.g., phenyl), substituted or unsubstituted heteroaryl (e.g., pyridine (2, 3, and 4-pyridine);
each R₈ is independently [CH₂]ₚ
   wherein p is between 1 and 10 (e.g., 1, 2);
R₉ is [CH]_{q}, [C]_{q}
   wherein q is between 2 and 10;
R₁₀ and R₁₁ are each independently H, OH, COOH, C₁-C₅ substituted or unsubstituted linear or branched alkyl (e.g., methyl, ethyl, CH₂-cyclopropyl, CH₂—CH₂—O—CH₃), C₃-C₈ substituted or unsubstituted cycloalkyl (e.g., cyclopropyl), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;
or R₁₀ and R₁₁ are joined to form a substituted or unsubstituted 3-8 membered heterocyclic ring (e.g., piperazine, piperidine),
n is an integer between 0 and 4 (e.g., 1, 2);
or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In some embodiments, at least one of R₇, R₇′, R₇″, R₇″″ and R₇″″ is not H. In some embodiments, at least two of R₇, R₇′, R₇″, R₇′″ and R₇″″ are not H. In some embodiments, at least one of R₇′, R₇″, R₇′″ and R₇″″ is not H. In some embodiments, at least two of R₇′, R₇″, R₇′″ and R₇″″ are not H. In some embodiments, at least two of R₇′, R₇″, R₇′″ and R₇″″ are different than each other. In some embodiments, at least two of R₇′, R₇″, R₇′″ and R₇″″ are not H and are different than each other.

In some embodiments, at least one of X₂, X₃, and X₄ is C(R). In some embodiments, X₁₁ is N. In some embodiments, X₁₂ is not S. In some embodiments, at least one of X₂, X₃, and X₄ is C(R); X₁₁ is N; or X₁₂ is not S. In some embodiments, R is not H.

In various embodiments, this invention is directed to a compound represented by the structure of formula I(d):

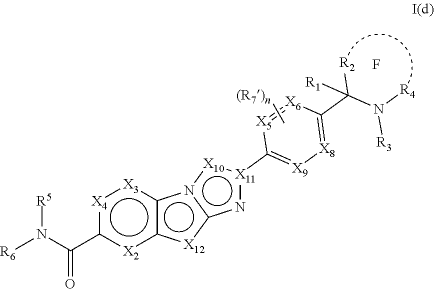

wherein
   X₂, X₃, and X₄, are each independently CH, C(R) or N (e.g., C(CH₃), C(O—CH₂-cyclopropyl), C(O—CH₂-methylcyclobutyl), C(NH—CH₂-cyclopropyl), C(isopropoxy), CO—CH(CH₃)—CH₂—O—CH₃), C(CH₂CH₃), C-iPr, C—CH₂-cyclopropyl, C(OCH₃), C(OCH₂CH₃), C(O—(CH₂)₂O—CH₃, C(OCHF₂), C(Cl), C(C(O)CH3), C(O—CH₂CH₂—O—CH₃), C(OH));
   X₅, X₆, X₇, X₈ and X₉ are each independently nitrogen or carbon atoms;
   X₁₀ is N, CH, C(R), or C═O; wherein if X₁₀ is C═O then X₁₁ is N (e.g., C(CH₂—OH), C(CH₂—CH₂—OH), C(NH—CH₂-cyclopropyl), C(COOH), C(CH₃), C(cyclopropyl), C(isopropoxy));
   X₁₁ is N or C; wherein if X₁₁ is N then X₁₀ is C═O;
   X₁₂ is S, SO₂, O, NH, N(R), N—OH, CH═CH, CH═CH(R), C(R)═CH, N═CH, N═C(R), CH═N or C(R)═N (e.g., N—CH₂—COOH, N—CH₂—CH₂—OH, N—CH₃, N—CH₂CH₃, N-iPr, N-cyclopropyl, N—CH₂-cyclopropyl);
   Ring F is absent or is a substituted or unsubstituted, saturated or unsaturated, 3-8 membered heterocyclic or carbocyclic ring (e.g., pyrrolidine, 1-methylpyrrolidine, pyrrolidin-2-one, pyrrolidin-3-ol, morpholine, pyridine, piperidine, tetrahydrofurane, tetrahydrothiophene, cyclopropyl, oxetane, imidazole, pyrimidine, triazole, oxadiazole, pyrazole);
   R₁ and R₂ are each independently H, F, Cl, Br, I, OH, SH, or CF₃, substituted or unsubstituted C₁-C₅ alkyl, C₁-C₅ linear or branched, or C₃-C₅ cyclic haloalkyl, substituted or unsubstituted C₁-C₅ linear or branched, or C₃-C₅ cyclic alkoxy;
   or R₁ and R₂ are joined to form a C═O, or a 3-8 membered carbocyclic or heterocyclic ring (e.g., cyclopropyl);
   or R₂ and R₄ are joined to form Ring F as defined above (e.g., pyrrolidine, pyrrolidin-2-one, pyrrolidin-3-ol, morpholine, pyridine, pyrimidine, triazole, oxadiazole, pyrazole), wherein if Ring F is aromatic, then R₁ and/or R₃ are absent;
   R₃ and R₄ are each independently H, Me, substituted or unsubstituted C₁-C₅ alkyl (e.g., methoxyethylene, methylaminoethyl, aminoethyl), —R₈—O—R₁₀ (e.g., (CH₂)₂O—CH₃), R₈—N(R₁₀)(R₁₁) (e.g., (CH₂)₂—NH(CH₃)), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀;
or R₃ and R₄ are joined to form a 3-8 membered heterocyclic ring (e.g., pyrrolidine, pyrrolidone, 2-oxopyrrolidine, piperidine, morpholine, piperazine, imidazole);
R₅ is H or C₁-C₅ linear or branched alkyl (e.g. methyl);
R₆ is H, F, Cl, Br, I, OH, SH, R₈—OH, R₈—SH, —R₈—O—R₁₀ (e.g., CH₂—O—CH₃, (CH₂)₂O—CH₃ (CH₂)₃O—CH₃, (CH₂)₂O—CH(CH₃)₂), R₈—S—R₁₀ (e.g., (CH₂)₃—S—(CH₂)₂CH₃), R₈—NHC(O)—R₁₀, —O—R₈-R₁₀, R₈-(substituted or unsubstituted C₃-C₈ cycloalkyl) (e.g., CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted, saturated, unsaturated or aromatic, single, fused or spiro 3-10 membered heterocyclic ring) (e.g., (CH₂)₃-piperidine, (CH₂)₃-4-fluoro-piperidine, (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-oxa-azaspirodecane, (CH₂)₃-dimethylpyrazole, CH₂-2-oxo-methylpyrrolidine, CH₂-methyl-azetidine, CH₂-azaspiroheptane), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁) (e.g., (CH₂)₂—NH₂, (CH₂)₃—N(CH₂CH₃)₂, (CH₂)₃—N(CH(CH₃)₂)₂, (CH₂)₃-piperidine, (CH₂)₃-4-fluoro-piperidine, (CH₂)₃-4-cyano-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃)), R₉—R₈—N(R₁₀)(R₁₁) (e.g., (CH₂)₂—C(O)-piperidine), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)CH₃), C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclobutyl, cyclohexyl, methoxycyclopropyl, methylcyclobutyl, aminomethyl-cyclobutyl, methoxycyclobutyl, 2,3-dihydro-1H-indenol), R₈-(substituted or unsubstituted C₃-C₈ cycloalkyl), substituted or unsubstituted 3-8 membered heterocyclic ring (e.g., piperidine, 1-methyl-piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), substituted or unsubstituted benzyl;
or R₆ and R₅ are joined to for a substituted or unsubstituted 5-8 membered heterocyclic ring (e.g., azepane, piperazine, 2-(piperazin-1-yl)acetamide;
or R₆ is represented by the structure of formula B or Bi:

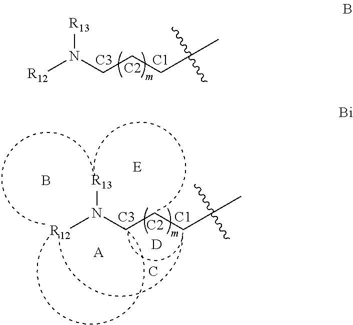

wherein
m is 0 or 1; and
   R₁₂ is R₂₀ or C₁-C₅ C(O)-alkyl, and R₁₃ is R₃₀; or
   R₁₂ and R₁₃ are both H;
   R₁₂ and R₁₃ are each independently H or substituted or unsubstituted C₁-C₅ alkyl (e.g., ethyl, trifluoroethyl);
   R₁₂ and C3 are joined to form ring A and R₁₃ is R₃₀; or
   R₁₂ and R₁₃ are joined to form ring B; or
   R₁₂ and C1 are joined to form ring C and R₁₃ is R₃₀; or
   C1 and C3 are joined to form ring D and R₁₂ and R₁₃ are each independently R₃₀; or
   R₁₃ and C2 are joined to form ring E, m is 1, and R₁₂ is R₃₀; or
   R₁₂ and R₁₃ are joined to form ring B and C1 and C3 are joined to form ring D;
   wherein
      Ring A, C and E are each independently a substituted or unsubstituted single spiro or fused 3-8 membered heterocyclic ring (e.g., A: pyrrolidine, methylpyrrolidine, ethylpyrrolidine); C: piperidine, pyrrolidine, methyl-2-oxopyrrolidine, pyran-pyrrolidine, methyl-azetidine, azabicyclooctane, 2-azabicyclo[2.1.1]hexane, 2-azaspiro[3.3]heptane; E: pyrrolidine, azetidine, ethylpyrrolidine, oxopyrrolidine, methylpiperidine);
      Ring B is a substituted or unsubstituted single, spiro or fused 3-8 membered heterocyclic ring (B: piperidine, piperidin-2-one, 4-fluoropiperidin-2-one, piperidine-4-carbonitrile, 4-fluoropiperidine, 4-fluoro-2-methylpiperidine, methyl-piperidin, fluoropiperidine, difluoropiperidine, pyrrolidine, piperazine, methylpyrrolidine, thiomorpholine 1,1-dioxide, 2-oxa-6-azaspiro[3.3]heptane, methyl-piperazine, dimethyl-pyrazole, imidazole, 2-methyl-2,5-diazabicyclo[2.2.1]heptane, hydroxymethyl-pyrrolidine, diazabicyclo[2.2.1]heptane, 6-fluoro-3-azabicyclo[3.1.1]heptane; and
      Ring D is a substituted or unsubstituted C₃-C₈ cycloalkyl (e.g., cyclobutane, cyclohexane);
R₇′ is H, F, Cl, Br, I, OH, O—R₂₀, SH, R₈—OH, R₈—SH, —R₈—O—R₁₀, R₈—(C₃-C₅ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclohexyl), substituted or unsubstituted 3-8 membered heterocyclic ring (e.g., morpholine, pyran, oxetane, pyrrolidine, imidazole, piperazine, piperidine, dioxazole, 2-oxopyrrolidine), substituted or unsubstituted aryl (e.g., phenyl), substituted or unsubstituted benzyl;
R₂₀ is represented by the following structure:

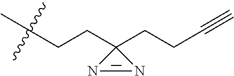 [SMILES: *CCC1(CCC#C)N=N1]

R is H, F, Cl, Br, I, OH, SH, COOH, CO(R₁₀) (e.g., C(O)CH₃), NH(R₁₀), NH—CH₂-cyclopropyl, N(R₁₀)(R₁₁), CF₃, CN, NO₂, C₁-C₅ linear or branched, substituted or unsubstituted alkyl (e.g., methyl, ethyl, iPr, CH₂-cyclopropyl, CH₂—OH, CH₂—CH₂—OH, CH₂—CH₂—O—CH₂—CH₂—O—CH₃, CH₂—O—CH₂—CH₂-O—CH₃), C₃-C₈ substituted or unsubstituted cycloalkyl, cyclopropyl, substituted or unsubstituted C₁-C₅ linear or branched alkoxy, (e.g., methoxy, ethoxy, O—(CH₂CH₃), OCHF₂, O—(CH₂)₂O—CH₃, isopropoxy, O—(CH₂)-cyclopropyl, O—CH₂-methylcyclobutyl, O—CH₂-3-methyloxetane, O—CH(CH₃)—CH₂—O—CH₃) C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, O—R₈-R₁₀ (e.g. O—(CH₂)₂O—CH₃, O—(CH₂CH₃), O—(CH₂)-cyclopropyl), —R₈—R₁₀ (e.g., (CH₂)-cyclopropyl, (CH₂)₂O—CH₃, (CH₂)—OH, (CH₂)₂—OH, (CH₂)—COOH), substituted or unsubstituted aryl (e.g., phenyl), substituted or unsubstituted heteroaryl (e.g., pyridine (2, 3, and 4-pyridine);
R₃₀ is H, R₂₀, F, Cl, Br, I, OH, SH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);
each R₈ is independently [CH₂]ₚ
   wherein p is between 1 and 10 (e.g., 1, 2);
R₉ is [CH]_{q}, [C]_{q}
   wherein q is between 2 and 10;
R₁₀ and R₁₁ are each independently H, OH, COOH, C₁-C₅ substituted or unsubstituted linear or branched alkyl (e.g., methyl, ethyl, CH₂-cyclopropyl, CH₂—CH₂—O—CH₃), C₃-C₈ substituted or unsubstituted cycloalkyl (e.g., cyclopropyl), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;
or R₁₀ and R₁₁ are joined to form a substituted or unsubstituted 3-8 membered heterocyclic ring (e.g., piperazine, piperidine),
n is an integer between 0 and 4 (e.g., 1, 2);
or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In some embodiments, at least one of X₂, X₃, and X₄ is C(R). In some embodiments, X₁₁ is N. In some embodiments, X₁₂ is not S. In some embodiments, at least one of X₂, X₃, and X₄ is C(R); X₁₁ is N; or X₁₂ is not S. In some embodiments, R is not H. In some embodiments, if R₁ and R₂ are joined to form a C═O, then at least one of X₂, X₃, X₄, and X₁₀ is not CH;

In various embodiments, this invention is directed to a compound represented by the structure of formula I(e):

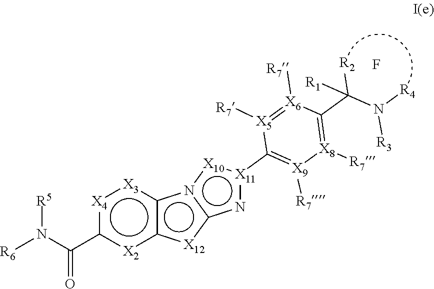

wherein
   X₂, X₃, and X₄, are each independently CH, C(R) or N (e.g., C(CH₃), CO—CH₂-cyclopropyl), CO—CH₂-methylcyclobutyl), C(NH—CH₂-cyclopropyl), C(isopropoxy), CO—CH(CH₃)—CH₂—O—CH₃), C(CH₂CH₃), C-iPr, C—CH₂-cyclopropyl, C(OCH₃), C(OCH₂CH₃), C(O—(CH₂)₂O—CH₃, C(OCHF₂), C(Cl), C(C(O)CH3), CO—CH₂CH₂—O—CH₃), C(OH));
   X₅, X₆, X₇, X₈ and X, are each independently nitrogen or carbon atoms;
      wherein if either one of X₅, X₆, X₈ and X₉ is nitrogen, then the respective R₇′, R₇″, R₇′″, and R₇″″ substitution is absent;
   X₁₀ is N, CH, C(R), or C═O; wherein if X₁₀ is C═O then X₁₁ is N (e.g., C(CH₂—OH), C(CH₂—CH₂—OH), C(NH—CH₂-cyclopropyl), C(COOH), C(CH₃), C(cyclopropyl), C(isopropoxy));
   X₁₁ is N or C; wherein if X₁₁ is N then X₁₀ is C═O;
   X₁₂ is S, SO₂, O, NH, N(R), N—OH, CH═CH, CH═CH(R), C(R)═CH, N═CH, N═C(R), CH═N or C(R)═N (e.g., N—CH₂—COOH, N—CH₂—CH₂—OH, N—CH₃, N—CH₂CH₃, N-iPr, N-cyclopropyl, N—CH₂-cyclopropyl);
   Ring F is absent or is a substituted or unsubstituted, saturated or unsaturated, 3-8 membered heterocyclic or carbocyclic ring (e.g., pyrrolidine, 1-methylpyrrolidine, pyrrolidin-2-one, pyrrolidin-3-ol, morpholine, pyridine, piperidine, tetrahydrofurane, tetrahydrothiophene, cyclopropyl, oxetane, imidazole, pyrimidine, triazole, oxadiazole, pyrazole);
   R₁ and R₂ are each independently H, F, Cl, Br, I, OH, SH, or CF₃, substituted or unsubstituted C₁-C₅ alkyl, C₁-C₅ linear or branched, or C₃-C₈ cyclic haloalkyl, substituted or unsubstituted C₁-C₅ linear or branched, or C₃-C₈ cyclic alkoxy;
   or R₁ and R₂ are joined to form a C═O, or a 3-8 membered carbocyclic or heterocyclic ring (e.g., cyclopropyl);
   or R₂ and R₄ are joined to form Ring F as defined above (e.g., pyrrolidine, pyrrolidin-2-one, pyrrolidin-3-ol, morpholine, pyridine, pyrimidine, triazole, oxadiazole, pyrazole), wherein if Ring F is aromatic, then R₁ and/or R₃ are absent;
   R₃ and R₄ are each independently H, Me, substituted or unsubstituted C₁-C₅ alkyl (e.g., methoxyethylene, methylaminoethyl, aminoethyl), —R₈—O—R₁₀ (e.g., (CH₂)₂O—CH₃), R₈—N(R₁₀)(R₁₁) (e.g., (CH₂)₂—NH(CH₃)), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀;
   or R₃ and R₄ are joined to form a 3-8 membered heterocyclic ring (e.g., pyrrolidine, pyrrolidone, 2-oxopyrrolidine, piperidine, morpholine, piperazine, imidazole);
   R₅ is H or C₁-C₅ linear or branched alkyl (e.g. methyl);
   R₆ is H, F, Cl, Br, I, OH, SH, R₈—OH, R₈—SH, —R₈—O—R₁₀ (e.g., CH₂—O—CH₃, (CH₂)₂O—CH₃ (CH₂)₃O—CH₃, (CH₂)₂O—CH(CH₃)₂), R₈—S—R₁₀ (e.g., (CH₂)₃—S—(CH₂)₂CH₃), R₈—NHC(O)—R₁₀, —O—R₈-R₁₀, R₈-(substituted or unsubstituted C₃-C₈ cycloalkyl) (e.g., CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted, saturated, unsaturated or aromatic, single, fused or spiro 3-10 membered heterocyclic ring) (e.g., (CH₂)₃-piperidine, (CH₂)₃-4-fluoro-piperidine, (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-oxa-azaspirodecane, (CH₂)₃-dimethylpyrazole, CH₂-2-oxo-methylpyrrolidine, CH₂-methyl-azetidine, CH₂-azaspiroheptane), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁) (e.g., (CH₂)₂—NH₂, (CH₂)₃—N(CH₂CH₃)₂, (CH₂)₃—N(CH(CH₃)₂)₂, (CH₂)₃-piperidine, (CH₂)₃-4-fluoro-piperidine, (CH₂)₃-4-cyano-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃)), R₉—R₈—N(R₁₀)(R₁₁) (e.g., (CH₂)₂—C(O)-piperidine), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)CH₃), C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclobutyl, cyclohexyl, methoxycyclopropyl, methylcyclobutyl, aminomethyl-cyclobutyl, methoxycyclobutyl, 2,3-dihydro-1H-indenol), R₈-(substituted or unsubstituted C₃-C₈ cycloalkyl), substituted or unsubstituted 3-8 membered heterocyclic ring (e.g., piperidine, 1-methyl-piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), substituted or unsubstituted benzyl;
   or R₆ and R₅ are joined to for a substituted or unsubstituted 5-8 membered heterocyclic ring (e.g., azepane, piperazine, 2-(piperazin-1-yl)acetamide;
   or R₆ is represented by the structure of formula B or Bi:

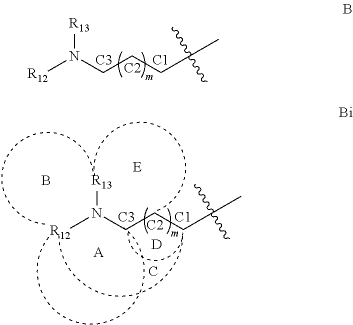

wherein
m is 0 or 1; and
   R₁₂ is R₂₀ or C₁-C₅ C(O)-alkyl, and R₁₃ is R₃₀; or
   R₁₂ and R₁₃ are both H;
   R₁₂ and R₁₃ are each independently H or substituted or unsubstituted C₁-C₅ alkyl (e.g., ethyl, trifluoroethyl);
   R₁₂ and C3 are joined to form ring A and R₁₃ is R₃₀; or
   R₁₂ and R₁₃ are joined to form ring B; or
   R₁₂ and C1 are joined to form ring C and R₁₃ is R₃₀; or
   C1 and C3 are joined to form ring D and R₁₂ and R₁₃ are each independently R₃₀; or
   R₁₃ and C2 are joined to form ring E, m is 1, and R₁₂ is R₃₀; or
   R₁₂ and R₁₃ are joined to form ring B and C1 and C3 are joined to form ring D;
   wherein
      Ring A, C and E are each independently a substituted or unsubstituted single spiro or fused 3-8 membered heterocyclic ring (e.g., A: pyrrolidine, methylpyrrolidine, ethylpyrrolidine); C: piperidine, pyrrolidine, methyl-2-oxopyrrolidine, pyran-pyrrolidine, methyl-azetidine, azabicyclooctane, 2-azabicyclo[2.1.1]hexane, 2-azaspiro[3.3]heptane; E: pyrrolidine, azetidine, ethylpyrrolidine, oxopyrrolidine, methylpiperidine);
      Ring B is a substituted or unsubstituted single, spiro or fused 3-8 membered heterocyclic ring (B: piperidine, piperidin-2-one, 4-fluoropiperidin-2-one, piperidine-4-carbonitrile, 4-fluoropiperidine, 4-fluoro-2-methylpiperidine, methyl-piperidin, fluoropiperidine, difluoropiperidine, pyrrolidine, piperazine, methylpyrrolidine, thiomorpholine 1,1-dioxide, 2-oxa-6-azaspiro[3.3]heptane, methyl-piperazine, dimethyl-pyrazole, imidazole, 2-methyl-2,5-diazabicyclo[2.2.1]heptane, hydroxymethyl-pyrrolidine, diazabicyclo[2.2.1]heptane, 6-fluoro-3-azabicyclo[3.1.1]heptane; and
      Ring D is a substituted or unsubstituted C₃-C₈ cycloalkyl (e.g., cyclobutane, cyclohexane);
R₇₉′, R₇″, R₇′″ and R₇″″ are each independently H, F, Cl, Br, I, OH, O—R₂₀, SH, R₈—OH, R₈—SH, —R₈—O—R₁₀, R₈—(C₃-C₅ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₈ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclohexyl), substituted or unsubstituted 3-8 membered heterocyclic ring (e.g., morpholine, pyran, oxetane, pyrrolidine, tetrahydrofuran, imidazole, piperazine, piperidine, dioxazole, 2-oxopyrrolidine), substituted or unsubstituted aryl (e.g., phenyl), substituted or unsubstituted benzyl;
or R₇′ and R₇″ are joined to form a 3-8 membered substituted or unsubstituted, saturated, unsaturated or aromatic, carbocyclic or heterocyclic ring (e.g., cyclopentyl, cyclohexyl, piperidine, tetrahydrofuran, tetrahydropyran, pyrrolidine);
or R₇′″ and R₇″″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
R₂₀ is represented by the following structure:

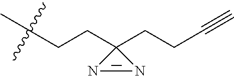 [SMILES: *CCC1(CCC#C)N=N1]

R is H, F, Cl, Br, I, OH, SH, COOH, CO(R₁₀) (e.g., C(O)CH₃), NH(R₁₀), NH—CH₂-cyclopropyl, N(R₁₀)(R₁₁), CF₃, CN, NO₂, C₁-C₅ linear or branched, substituted or unsubstituted alkyl (e.g., methyl, ethyl, iPr, CH₂-cyclopropyl, CH₂—OH, CH₂—CH₂—OH, CH₂—CH₂—O—CH₂—CH₂—O—CH₃, CH₂—O—CH₂—CH₂-O—CH₃), C₃-C₈ substituted or unsubstituted cycloalkyl, cyclopropyl, substituted or unsubstituted C₁-C₅ linear or branched alkoxy, (e.g., methoxy, ethoxy, O—(CH₂CH₃), OCHF₂, O—(CH₂)₂O—CH₃, isopropoxy, O—(CH₂)-cyclopropyl, O—CH₂-methylcyclobutyl, O—CH₂-3-methyloxetane, O—CH(CH₃)—CH₂—O—CH₃) C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, O—R₈-R₁₀ (e.g. O—(CH₂)₂O—CH₃, O—(CH₂CH₃), O—(CH₂)-cyclopropyl), —R₈—R₁₀ (e.g., (CH₂)-cyclopropyl, (CH₂)₂O—CH₃, (CH₂)—OH, (CH₂)₂—OH, (CH₂)—COOH), substituted or unsubstituted aryl (e.g., phenyl), substituted or unsubstituted heteroaryl (e.g., pyridine (2, 3, and 4-pyridine);
R₃₀ is H, R₂₀, F, Cl, Br, I, OH, SH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);
each R₈ is independently [CH₂]ₚ
   wherein p is between 1 and 10 (e.g., 1, 2);
R₉ is [CH]_{q}, [C]_{q}
   wherein q is between 2 and 10;
R₁₀ and R₁₁ are each independently H, OH, COOH, C₁-C₅ substituted or unsubstituted linear or branched alkyl (e.g., methyl, ethyl, CH₂-cyclopropyl, CH₂—CH₂—O—CH₃), C₃-C₈ substituted or unsubstituted cycloalkyl (e.g., cyclopropyl), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;
or R₁₀ and R₁₁ are joined to form a substituted or unsubstituted 3-8 membered heterocyclic ring (e.g., piperazine, piperidine),
n is an integer between 0 and 4 (e.g., 1, 2);
or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In some embodiments, at least one of R₇′, R₇″, R₇′″ and R₇″″ is not H. In some embodiments, at least two of R₇′, R₇″, R₇′″ and R₇″″ are not H. In some embodiments, at least two of R₇′, R₇″, R₇′″ and R₇″″ are different than each other. In some embodiments, at least two of R₇′, R₇″, R₇′″ and R₇″″ are not H and are different than each other.

In some embodiments, at least one of X₂, X₃, and X₄ is C(R). In some embodiments, X₁₁ is N. In some embodiments, X₁₂ is not S. In some embodiments, at least one of X₂, X₃, and X₄ is C(R); X₁₁ is N; or X₁₂ is not S. In some embodiments, R is not H. In some embodiments, if R₁ and R₂ are joined to form a C═O, then at least one of X₂, X₃, X₄, and X₁₀ is not CH;

In various embodiments, this invention is directed to a compound represented by the structure of formula I(f):

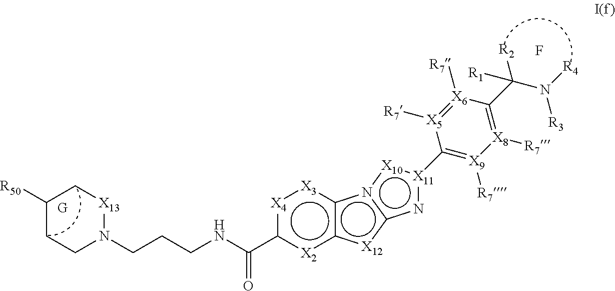

wherein
   X₂, X₃, and X₄, are each independently CH, C(R) or N (e.g., C(CH₃), C(O—CH₂-cyclopropyl), C(O—CH₂-methylcyclobutyl), C(NH—CH₂-cyclopropyl), C(isopropoxy), C(O—CH(CH₃)—CH₂—O—CH₃), C(CH₂CH₃), C-iPr, C—CH₂-cyclopropyl, C(OCH₃), C(OCH₂CH₃), C(O—(CH₂)₂O—CH₃, C(OCHF₂), C(Cl), C(C(O)CH3), C(O—CH₂CH₂—O—CH₃), C(OH));
   X₅, X₆, X₇, X₈ and X₉ are each independently nitrogen or carbon atoms;
      wherein if either one of X₅, X₆, X₈ and X₉ is nitrogen, then the respective R₇′, R₇″, R₇″, and R₇″″ substitution is absent;
   X₁₀ is N, CH, C(R), or C═O; wherein if X₁₀ is C═O then X₁₁ is N (e.g., C(CH₂—OH), C(CH₂—CH₂—OH), C(NH—CH₂-cyclopropyl), C(COOH), C(CH₃), C(cyclopropyl), C(isopropoxy));
   X₁₁ is N or C; wherein if X₁₁ is N then X₁₀ is C═O;
   X₁₂ is S, SO₂, O, NH, N(R), N—OH, CH═CH, CH═CH(R), C(R)═CH, N═CH, N═C(R), CH═N or C(R)═N (e.g., N—CH₂—COOH, N—CH₂—CH₂—OH, N—CH₃, N—CH₂CH₃, N-iPr, N-cyclopropyl, N—CH₂-cyclopropyl);
   X₁₃ is CH₂, CH(R) (e.g., CH—CH₃), C(R)₂, or C═O;
   Ring G is absent or is a substituted or unsubstituted 3-8 membered carbocyclic or heterocyclic ring (e.g., cyclobutane, cyclopentane, cyclohexane);
   Ring F is absent or is a substituted or unsubstituted, saturated or unsaturated, 3-8 membered heterocyclic or carbocyclic ring (e.g., pyrrolidine, 1-methylpyrrolidine, pyrrolidin-2-one, pyrrolidin-3-ol, morpholine, pyridine, piperidine, tetrahydrofurane, tetrahydrothiophene, cyclopropyl, oxetane, imidazole, pyrimidine, triazole, oxadiazole, pyrazole);
   R₁ and R₂ are each independently H, F, Cl, Br, I, OH, SH, or CF₃, substituted or unsubstituted C₁-C₅ alkyl, C₁-C₅ linear or branched, or C₃-C₈ cyclic haloalkyl, substituted or unsubstituted C₁-C₅ linear or branched, or C₃-C₈ cyclic alkoxy;
   or R₁ and R₂ are joined to form a C═O, or a 3-8 membered carbocyclic or heterocyclic ring (e.g., cyclopropyl);
   or R₂ and R₄ are joined to form Ring F as defined above (e.g., pyrrolidine, pyrrolidin-2-one, pyrrolidin-3-ol, morpholine, pyridine, pyrimidine, triazole, oxadiazole, pyrazole), wherein if Ring F is aromatic, then R₁ and/or R₃ are absent;
   R₃ and R₄ are each independently H, Me, substituted or unsubstituted C₁-C₅ alkyl (e.g., methoxyethylene, methylaminoethyl, aminoethyl), —R₈—O—R₁₀ (e.g., (CH₂)₂O—CH₃), R₈—N(R₁₀)(R₁₁) (e.g., (CH₂)₂—NH(CH₃)), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀;
   or R₃ and R₄ are joined to form a 3-8 membered heterocyclic ring (e.g., pyrrolidine, pyrrolidone, 2-oxopyrrolidine, piperidine, morpholine, piperazine, imidazole);
   R₇′, R₇″, R₇′″ and R₇″″ are each independently H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₈ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclohexyl), substituted or unsubstituted 3-8 membered heterocyclic ring (e.g., morpholine, pyran, oxetane, pyrrolidine, tetrahydrofuran, imidazole, piperazine, piperidine, dioxazole, 2-oxopyrrolidine), substituted or unsubstituted aryl (e.g., phenyl), substituted or unsubstituted benzyl;
   or R₇′ and R₇″ are joined to form a 3-8 membered substituted or unsubstituted, saturated, unsaturated or aromatic, carbocyclic or heterocyclic ring (e.g., cyclopentyl, cyclohexyl, piperidine, tetrahydrofuran, tetrahydropyran, pyrrolidine);
   or R₇′″ and R₇″″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
   R₂₀ is represented by the following structure:

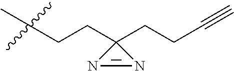 [SMILES: *CCC1(CCC#C)N=N1]

R₃₀ is H, R₂₀, F, Cl, Br, I, OH, SH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);
R₅₀ is H, R₂₀, F, Cl, Br, I, OH, SH, N(R)₂, NH(R₁₀), N(R₁₀)(R₁₁), CF₃, CN, NO₂, C₁-C₅ linear or branched, substituted or unsubstituted alkyl C₁-C₅ linear or branched alkoxy, C₁-C₅ linear or branched haloalkyl, —R₈—R₁₀ (e.g., (CH₂)₂O—CH₃), substituted or unsubstituted aryl (e.g., phenyl), substituted or unsubstituted heteroaryl (e.g., pyridine (2, 3, and 4-pyridine);
R is H, F, Cl, Br, I, OH, SH, COOH, CO(R₁₀) (e.g., C(O)CH₃), NH(R₁₀), NH—CH₂-cyclopropyl, N(R₁₀)(R₁₁), CF₃, CN, NO₂, C₁-C₅ linear or branched, substituted or unsubstituted alkyl (e.g., methyl, ethyl, iPr, CH₂-cyclopropyl, CH₂—OH, CH₂—CH₂—OH, CH₂—CH₂—O—CH₂—CH₂—O—CH₃, CH₂—O—CH₂—CH₂-O—CH₃), C₃-C₈ substituted or unsubstituted cycloalkyl, cyclopropyl, substituted or unsubstituted C₁-C₅ linear or branched alkoxy, (e.g., methoxy, ethoxy, O—(CH₂CH₃), OCHF₂, O—(CH₂)₂O—CH₃, isopropoxy, O—(CH₂)-cyclopropyl, O—CH₂-methylcyclobutyl, O—CH₂-3-methyloxetane, O—CH(CH₃)—CH₂—O—CH₃) C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, O—R₈-R₁₀ (e.g. O—(CH₂)₂O—CH₃, O—(CH₂CH₃), O—(CH₂)-cyclopropyl), —R₈—R₁₀ (e.g., (CH₂)-cyclopropyl, (CH₂)₂O—CH₃, (CH₂)—OH, (CH₂)₂—OH, (CH₂)—COOH), substituted or unsubstituted aryl (e.g., phenyl), substituted or unsubstituted heteroaryl (e.g., pyridine (2, 3, and 4-pyridine);
each R₈ is independently [CH₂]ₚ
   wherein p is between 1 and 10 (e.g., 1, 2);
R₉ is [CH]_{q}, [C]_{q}
   wherein q is between 2 and 10;
R₁₀ and R₁₁ are each independently H, OH, COOH, C₁-C₅ substituted or unsubstituted linear or branched alkyl (e.g., methyl, ethyl, CH₂-cyclopropyl, CH₂—CH₂—O—CH₃), C₃-C₈ substituted or unsubstituted cycloalkyl (e.g., cyclopropyl), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;
or R₁₀ and R₁₁ are joined to form a substituted or unsubstituted 3-8 membered heterocyclic ring (e.g., piperazine, piperidine),
n is an integer between 0 and 4 (e.g., 1, 2);
or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In some embodiments, at least one of R₇′, R₇″, R₇′″ and R₇″″ is not H. In some embodiments, at least two of R₇′, R₇″, R₇′″ and R₇″″ are not H. In some embodiments, at least two of R₇′, R₇″, R₇′″ and R₇″″ are different than each other. In some embodiments, at least two of R₇′, R₇″, R₇′″ and R₇″″ are not H and are different than each other. In some embodiments, if R₁ and R₂ are joined to form a C═O, then at least one of X₂, X₃, X₄, and X₁₀ is not CH;

In some embodiments, at least one of X₂, X₃, and X₄ is C(R). In some embodiments, X₁₁ is N. In some embodiments, X₁₂ is not S. In some embodiments, at least one of X₂, X₃, and X₄ is C(R); X₁₁ is N; or X₁₂ is not S. In some embodiments, R is not H.

In various embodiments, this invention is directed to a compound represented by the structure of formula I(g):

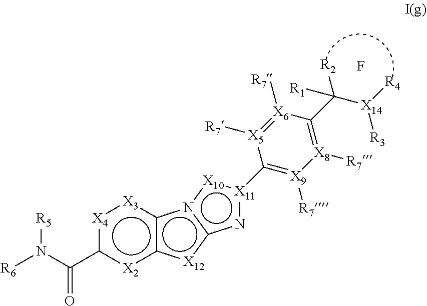

wherein
   X₂, X₃, and X₄, are each independently CH, C(R) or N (e.g., C(CH₃), C(O—CH₂-cyclopropyl), C(O—CH₂-methylcyclobutyl), C(NH—CH₂-cyclopropyl), C(isopropoxy), C(O—CH(CH₃)—CH₂—O—CH₃), C(CH₂CH₃), C-iPr, C—CH₂-cyclopropyl, C(OCH₃), C(OCH₂CH₃), C(O—(CH₂)₂—O—CH₃, C(OCHF₂), C(Cl), C(C(O)CH3), C(O—CH₂CH₂—O—CH₃), C(OH));
   X₅, X₆, X₇, X₈ and X₉ are each independently nitrogen or carbon atoms;
      wherein if either one of X₅, X₆, X₈ and X₉ is nitrogen, then the respective R₇′, R₇″, R₇′″, and R₇″″ substitution is absent;
   X₁₀ is N, CH, C(R), or C═O; wherein if X₁₀ is C═O then X₁₁ is N (e.g., C(CH₂—OH), C(CH₂—CH₂—OH), C(NH—CH₂-cyclopropyl), C(COOH), C(CH₃), C(cyclopropyl), C(isopropoxy));
   X₁₁ is N or C; wherein if X₁₁ is N then X₁₀ is C═O;
   X₁₂ is S, SO₂, O, NH, N(R), N—OH, CH═CH, CH═CH(R), C(R)═CH, N═CH, N═C(R), CH═N or C(R)═N (e.g., N—CH₂—COOH, N—CH₂—CH₂—OH, N—CH₃, N—CH₂CH₃, N-iPr, N-cyclopropyl, N—CH₂-cyclopropyl);
   X₁₄ is S, O, N or CH, wherein if X₁₄ is CH then Ring F is not absent and if X₁₄ is S or O then R₃ is absent;
   Ring F is absent or is a substituted or unsubstituted, saturated or unsaturated, 3-8 membered heterocyclic or carbocyclic ring (e.g., pyrrolidine, 1-methylpyrrolidine, pyrrolidin-2-one, pyrrolidin-3-ol, morpholine, pyridine, piperidine, tetrahydrofurane, tetrahydrothiophene, cyclopropyl, oxetane, imidazole, pyrimidine, triazole, oxadiazole, pyrazole);
   R₁ and R₂ are each independently H, F, Cl, Br, I, OH, SH, or CF₃, substituted or unsubstituted C₁-C₅ alkyl, C₁-C₅ linear or branched, or C₃-C₈ cyclic haloalkyl, substituted or unsubstituted C₁-C₅ linear or branched, or C₃-C₈ cyclic alkoxy;
   or R₁ and R₂ are joined to form a C═O, or a 3-8 membered carbocyclic or heterocyclic ring (e.g., cyclopropyl);
   or R₂ and R₄ are joined to form Ring F as defined above (e.g., pyrrolidine, pyrrolidin-2-one, pyrrolidin-3-ol, morpholine, pyridine, pyrimidine, triazole, oxadiazole, pyrazole), wherein if Ring F is aromatic, then R₁ and/or R₃ are absent;
   R₃ and R₄ are each independently H, Me, substituted or unsubstituted C₁-C₅ alkyl (e.g., methoxyethylene, methylaminoethyl, aminoethyl), —R₈—O—R₁₀ (e.g., (CH₂)₂O—CH₃), R₈—N(R₁₀)(R₁₁) (e.g., (CH₂)₂—NH(CH₃)), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀;
   or R₃ and R₄ are joined to form a 3-8 membered heterocyclic ring (e.g., pyrrolidine, pyrrolidone, 2-oxopyrrolidine, piperidine, morpholine, piperazine, imidazole);
   wherein if X₁₄ is S or O then R₃ is absent;
   R₅ is H or C₁-C₅ linear or branched alkyl (e.g. methyl);
   R₆ is H, F, Cl, Br, I, OH, SH, R₈—OH, R₈—SH, —R₈—O—R₁₀ (e.g., CH₂—O—CH₃, (CH₂)₂O—CH₃ (CH₂)₃O—CH₃, (CH₂)₂O—CH(CH₃)₂), R₈—S—R₁₀ (e.g., (CH₂)₃—S—(CH₂)₂CH₃), R₈—NHC(O)—R₁₀, —O—R₈-R₁₀, R₈-(substituted or unsubstituted C₃-C₈ cycloalkyl) (e.g., CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted, saturated, unsaturated or aromatic, single, fused or spiro 3-10 membered heterocyclic ring) (e.g., (CH₂)₃-piperidine, (CH₂)₃-4-fluoro-piperidine, (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-oxa-azaspirodecane, (CH₂)₃-dimethylpyrazole, CH₂-2-oxo-methylpyrrolidine, CH₂-methyl-azetidine, CH₂-azaspiroheptane), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁) (e.g., (CH₂)₂—NH₂, (CH₂)₃—N(CH₂CH₃)₂, (CH₂)₃—N(CH(CH₃)₂)₂, (CH₂)₃-piperidine, (CH₂)₃-4-fluoro-piperidine, (CH₂)₃-4-cyano-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃)), R₉—R₈—N(R₁₀)(R₁₁) (e.g., (CH₂)₂—C(O)-piperidine), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)CH₃), C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₅ cycloalkyl (e.g., cyclopropyl, cyclobutyl, cyclohexyl, methoxycyclopropyl, methylcyclobutyl, aminomethyl-cyclobutyl, methoxycyclobutyl, 2,3-dihydro-1H-indenol), R₈-(substituted or unsubstituted C₃-C₈ cycloalkyl), substituted or unsubstituted 3-8 membered heterocyclic ring (e.g., piperidine, 1-methyl-piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), substituted or unsubstituted benzyl;
   or R₆ and R₅ are joined to for a substituted or unsubstituted 5-8 membered heterocyclic ring (e.g., azepane, piperazine, 2-(piperazin-1-yl)acetamide;
   or R₆ is represented by the structure of formula B or Bi:

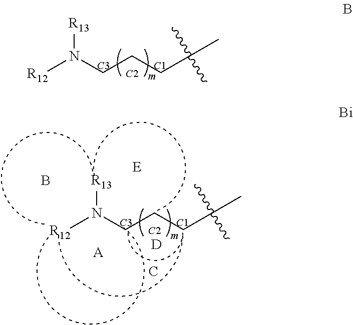

wherein
m is 0 or 1; and
   R₁₂ is R₂₀ or C₁-C₅ C(O)-alkyl, and R₁₃ is R₃₀; or
   R₁₂ and R₁₃ are both H;
   R₁₂ and R₁₃ are each independently H or substituted or unsubstituted C₁-C₅ alkyl (e.g., ethyl, trifluoroethyl);
   R₁₂ and C3 are joined to form ring A and R₁₃ is R₃₀; or
   R₁₂ and R₁₃ are joined to form ring B; or
   R₁₂ and C1 are joined to form ring C and R₁₃ is R₃₀; or
   C1 and C3 are joined to form ring D and R₁₂ and R₁₃ are each independently R₃₀; or
   R₁₃ and C2 are joined to form ring E, m is 1, and R₁₂ is R₃₀; or
   R₁₂ and R₁₃ are joined to form ring B and C1 and C3 are joined to form ring D;
   wherein
      Ring A, C and E are each independently a substituted or unsubstituted single spiro or fused 3-8 membered heterocyclic ring (e.g., A: pyrrolidine, methylpyrrolidine, ethylpyrrolidine); C: piperidine, pyrrolidine, methyl-2-oxopyrrolidine, pyran-pyrrolidine, methyl-azetidine, azabicyclooctane, 2-azabicyclo[2.1.1]hexane, 2-azaspiro[3.3]heptane; E: pyrrolidine, azetidine, ethylpyrrolidine, oxopyrrolidine, methylpiperidine);
      Ring B is a substituted or unsubstituted single, spiro or fused 3-8 membered heterocyclic ring (B: piperidine, piperidin-2-one, 4-fluoropiperidin-2-one, piperidine-4-carbonitrile, 4-fluoropiperidine, 4-fluoro-2-methylpiperidine, methyl-piperidin, fluoropiperidine, difluoropiperidine, pyrrolidine, piperazine, methylpyrrolidine, thiomorpholine 1,1-dioxide, 2-oxa-6-azaspiro[3.3]heptane, methyl-piperazine, dimethyl-pyrazole, imidazole, 2-methyl-2,5-diazabicyclo[2.2.1]heptane, hydroxymethyl-pyrrolidine, diazabicyclo[2.2.1]heptane, 6-fluoro-3-azabicyclo[3.1.1]heptane; and
      Ring D is a substituted or unsubstituted C₃-C₈ cycloalkyl (e.g., cyclobutane, cyclohexane);
R₇′, R₇″, R₇′ and R₇″″ are each independently H, F, Cl, Br, I, OH, O—R₂₀, SH, R₈—OH, R₈—SH, —R₈—O—R₁₀, R₈—(C₃-C₅ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₈ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclohexyl), substituted or unsubstituted 3-8 membered heterocyclic ring (e.g., morpholine, pyran, oxetane, pyrrolidine, tetrahydrofuran, imidazole, piperazine, piperidine, dioxazole, 2-oxopyrrolidine), substituted or unsubstituted aryl (e.g., phenyl), substituted or unsubstituted benzyl;
or R₇′ and R₇″ are joined to form a 3-8 membered substituted or unsubstituted, saturated, unsaturated or aromatic, carbocyclic or heterocyclic ring (e.g., cyclopentyl, cyclohexyl, piperidine, tetrahydrofuran, tetrahydropyran, pyrrolidine);
or R₇′″ and R₇″″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
R₂₀ is represented by the following structure:

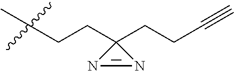 [SMILES: *CCC1(CCC#C)N=N1]

R is H, F, Cl, Br, I, OH, SH, COOH, CO(R₁₀) (e.g., C(O)CH₃), NH(R₁₀), NH—CH₂-cyclopropyl, N(R₁₀)(R₁₁), CF₃, CN, NO₂, C₁-C₅ linear or branched, substituted or unsubstituted alkyl (e.g., methyl, ethyl, iPr, CH₂-cyclopropyl, CH₂—OH, CH₂—CH₂—OH, CH₂—CH₂—O—CH₂—CH₂—O—CH₃, CH₂—O—CH₂—CH₂—O—CH₃), C₃-C₈ substituted or unsubstituted cycloalkyl, cyclopropyl, substituted or unsubstituted C₁-C₅ linear or branched alkoxy, (e.g., methoxy, ethoxy, O—(CH₂CH₃), OCHF₂, O—(CH₂)₂O—CH₃, isopropoxy, O—(CH₂)-cyclopropyl, O—CH₂-methylcyclobutyl, O—CH₂-3-methyloxetane, O—CH(CH₃)—CH₂—O—CH₃) C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, O—R₈-R₁₀ (e.g. O—(CH₂)₂O—CH₃, O—(CH₂CH₃), O—(CH₂)-cyclopropyl), —R₈—R₁₀ (e.g., (CH₂)-cyclopropyl, (CH₂)₂O—CH₃, (CH₂)—OH, (CH₂)₂—OH, (CH₂)—COOH), substituted or unsubstituted aryl (e.g., phenyl), substituted or unsubstituted heteroaryl (e.g., pyridine (2, 3, and 4-pyridine);
R₃₀ is H, R₂₀, F, Cl, Br, I, OH, SH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);
each R₈ is independently [CH₂]ₚ
   wherein p is between 1 and 10 (e.g., 1, 2);
R₉ is [CH]_{q}, [C]_{q}
   wherein q is between 2 and 10;
R₁₀ and R₁₁ are each independently H, OH, COOH, C₁-C₅ substituted or unsubstituted linear or branched alkyl (e.g., methyl, ethyl, CH₂-cyclopropyl, CH₂—CH₂—O—CH₃), C₃-C₈ substituted or unsubstituted cycloalkyl (e.g., cyclopropyl), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;
or R₁₀ and R₁₁ are joined to form a substituted or unsubstituted 3-8 membered heterocyclic ring (e.g., piperazine, piperidine),
n is an integer between 0 and 4 (e.g., 1, 2);
or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In some embodiments, at least one of R₇′, R₇″, R₇′″ and R₇″″ is not H. In some embodiments, at least two of R₇′, R₇″, R₇′″ and R₇″″ are not H. In some embodiments, at least two of R₇′, R₇″, R₇′″ and R₇″″ are different then each other. In some embodiments, at least two of R₇′, R₇″, R₇′″ and R₇″″ are not H and are different then each other.

In some embodiments, at least one of X₂, X₃, and X₄ is C(R). In some embodiments, X₁₁ is N. In some embodiments, X₁₂ is not S. In some embodiments, at least one of X₂, X₃, and X₄ is C(R); X₁₁ is N; or X₁₂ is not S. In some embodiments, R is not H. In some embodiments, if R₁ and R₂ are joined to form a C═O, then at least one of X₂, X₃, X₄, and X₁₀ is not CH;

In various embodiments, this invention is directed to a compound represented by the structure of formula I(h):

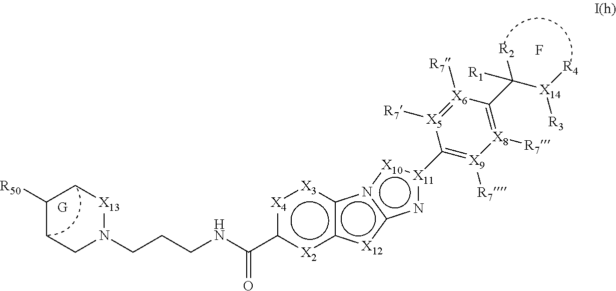

X₂, X₃, and X₄, are each independently CH, C(R) or N (e.g., C(CH₃), C(O—CH₂-cyclopropyl), C(O—CH₂-methylcyclobutyl), C(NH—CH₂-cyclopropyl), C(isopropoxy), C(O—CH(CH₃)—CH₂—O—CH₃), C(CH₂CH₃), C-iPr, C—CH₂-cyclopropyl, C(OCH₃), C(OCH₂CH₃), C(O—(CH₂)₂—O—CH₃, C(OCHF₂), C(Cl), C(C(O)CH3), C(O—CH₂CH₂—O—CH₃), C(OH));
X₅, X₆, X₇, X₈ and X₉ are each independently nitrogen or carbon atoms;
   wherein if either one of X₅, X₆, X₈ and X₉ is nitrogen, then the respective R₇′, R₇″, R₇′″, and R₇″″ substitution is absent;
X₁₀ is N, CH, C(R), or C═O; wherein if X₁₀ is C═O then X₁₁ is N (e.g., C(CH₂—OH), C(CH₂—CH₂—OH), C(NH—CH₂-cyclopropyl), C(COOH), C(CH₃), C(cyclopropyl), C(isopropoxy));
X₁₁ is N or C; wherein if X₁₁ is N then X₁₀ is C═O;
X₁₂ is S, SO₂, O, NH, N(R), N—OH, CH═CH, CH═CH(R), C(R)═CH, N═CH, N═C(R), CH═N or C(R)═N (e.g., N—CH₂—COOH, N—CH₂—CH₂—OH, N—CH₃, N—CH₂CH₃, N-iPr, N-cyclopropyl, N—CH₂-cyclopropyl);
X₁₃ is CH₂, CH(R) (e.g., CH—CH₃), C(R)₂, or C═O;
X₁₄ is S, O, N or CH, wherein if X₁₄ is CH then Ring F is not absent and if X₁₄ is S or O then R₃ is absent;
Ring G is absent or is a substituted or unsubstituted 3-8 membered carbocyclic or heterocyclic ring (e.g., cyclobutane, cyclopentane, cyclohexane);
Ring F is absent or is a substituted or unsubstituted, saturated or unsaturated, 3-8 membered heterocyclic or carbocyclic ring (e.g., pyrrolidine, 1-methylpyrrolidine, pyrrolidin-2-one, pyrrolidin-3-ol, morpholine, pyridine, piperidine, tetrahydrofurane, tetrahydrothiophene, cyclopropyl, oxetane, imidazole, pyrimidine, triazole, oxadiazole, pyrazole);
R₁ and R₂ are each independently H, F, Cl, Br, I, OH, SH, or CF₃, substituted or unsubstituted C₁-C₅ alkyl, C₁-C₅ linear or branched, or C₃-C₈ cyclic haloalkyl, substituted or unsubstituted C₁-C₅ linear or branched, or C₃-C₈ cyclic alkoxy;
or R₁ and R₂ are joined to form a C═O, or a 3-8 membered carbocyclic or heterocyclic ring (e.g., cyclopropyl);
or R₂ and R₄ are joined to form Ring F as defined above (e.g., pyrrolidine, pyrrolidin-2-one, pyrrolidin-3-ol, morpholine, pyridine, pyrimidine, triazole, oxadiazole, pyrazole), wherein if Ring F is aromatic, then R₁ and/or R₃ are absent;
R₃ and R₄ are each independently H, Me, substituted or unsubstituted C₁-C₅ alkyl (e.g., methoxyethylene, methylaminoethyl, aminoethyl), —R₈—O—R₁₀ (e.g., (CH₂)₂O—CH₃), R₈—N(R₁₀)(R₁₁) (e.g., (CH₂)₂—NH(CH₃)), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀;
or R₃ and R₄ are joined to form a 3-8 membered heterocyclic ring (e.g., pyrrolidine, pyrrolidone, 2-oxopyrrolidine, piperidine, morpholine, piperazine, imidazole);
R₇′, R₇″, R₇′″ and R₇″″ are each independently H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₈ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclohexyl), substituted or unsubstituted 3-8 membered heterocyclic ring (e.g., morpholine, pyran, oxetane, pyrrolidine, tetrahydrofuran, imidazole, piperazine, piperidine, dioxazole, 2-oxopyrrolidine), substituted or unsubstituted aryl (e.g., phenyl), substituted or unsubstituted benzyl;
or R₇′ and R₇″ are joined to form a 3-8 membered substituted or unsubstituted, saturated, unsaturated or aromatic, carbocyclic or heterocyclic ring (e.g., cyclopentyl, cyclohexyl, piperidine, tetrahydrofuran, tetrahydropyran, pyrrolidine);
or R₇′″ and R₇″″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
R₂₀ is represented by the following structure:

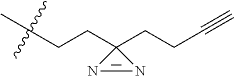 [SMILES: *CCC1(CCC#C)N=N1]

R₃₀ is H, R₂₀, F, Cl, Br, I, OH, SH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);
R₅₀ is H, R₂₀, F, Cl, Br, I, OH, SH, N(R)₂, NH(R₁₀), N(R₁₀)(R₁₁), CF₃, CN, NO₂, C₁-C₅ linear or branched, substituted or unsubstituted alkyl C₁-C₅ linear or branched alkoxy, C₁-C₅ linear or branched haloalkyl, —R₈—R₁₀ (e.g., (CH₂)₂O—CH₃), substituted or unsubstituted aryl (e.g., phenyl), substituted or unsubstituted heteroaryl (e.g., pyridine (2, 3, and 4-pyridine);
R is H, F, Cl, Br, I, OH, SH, COOH, CO(R₁₀) (e.g., C(O)CH₃), NH(R₁₀), NH—CH₂-cyclopropyl, N(R₁₀)(R₁₁), CF₃, CN, NO₂, C₁-C₅ linear or branched, substituted or unsubstituted alkyl (e.g., methyl, ethyl, iPr, CH₂-cyclopropyl, CH₂—OH, CH₂—CH₂—OH, CH₂—CH₂—O—CH₂—CH₂—O—CH₃, CH₂—O—CH₂—CH₂-O—CH₃), C₃-C₈ substituted or unsubstituted cycloalkyl, cyclopropyl, substituted or unsubstituted C₁-C₅ linear or branched alkoxy, (e.g., methoxy, ethoxy, O—(CH₂CH₃), OCHF₂, O—(CH₂)₂O—CH₃, isopropoxy, O—(CH₂)-cyclopropyl, O—CH₂-methylcyclobutyl, O—CH₂-3-methyloxetane, O—CH(CH₃)—CH₂—O—CH₃) C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, O—R₈-R₁₀ (e.g. O—(CH₂)₂O—CH₃, O—(CH₂CH₃), O—(CH₂)-cyclopropyl), —R₈—R₁₀ (e.g., (CH₂)-cyclopropyl, (CH₂)₂—O—CH₃, (CH₂)—OH, (CH₂)₂—OH, (CH₂)—COOH), substituted or unsubstituted aryl (e.g., phenyl), substituted or unsubstituted heteroaryl (e.g., pyridine (2, 3, and 4-pyridine);
each R₅ is independently [CH₂]ₚ
   wherein p is between 1 and 10 (e.g., 1, 2);
R₉ is [CH]_{q}, [C]_{q}
   wherein q is between 2 and 10;
R₁₀ and R₁₁ are each independently H, OH, COOH, C₁-C₅ substituted or unsubstituted linear or branched alkyl (e.g., methyl, ethyl, CH₂-cyclopropyl, CH₂—CH₂—O—CH₃), C₃-C₈ substituted or unsubstituted cycloalkyl (e.g., cyclopropyl), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;
or R₁₀ and R₁₁ are joined to form a substituted or unsubstituted 3-8 membered heterocyclic ring (e.g., piperazine, piperidine),
n is an integer between 0 and 4 (e.g., 1, 2);
or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In some embodiments, at least one of R₇′, R₇″, R₇′″ and R₇″″ is not H. In some embodiments, at least two of R₇′, R₇″, R₇′″ and R₇″″ are not H. In some embodiments, at least two of R₇′, R₇″, R₇′″ and R₇″″ are different then each other. In some embodiments, at least two of R₇′, R₇″, R₇′″ and R₇″″ are not H and are different then each other.

In some embodiments, at least one of X₂, X₃, and X₄ is C(R). In some embodiments, X₁₁ is N. In some embodiments, X₁₂ is not S. In some embodiments, at least one of X₂, X₃, and X₄ is C(R); X₁₁ is N; or X₁₂ is not S. In some embodiments, R is not H.

In various embodiments, this invention is directed to a compound represented by the structure of formula I(i):

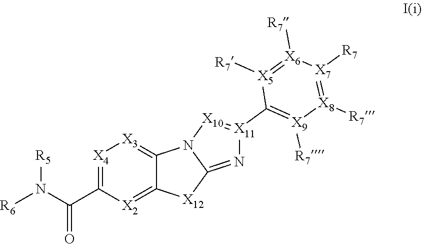

wherein
   X₂, X₃, and X₄, are each independently CH, C(R) or N (e.g., C(CH₃), C(O—CH₂-cyclopropyl), C(O—CH₂-methylcyclobutyl), C(NH—CH₂-cyclopropyl), C(isopropoxy), C(O—CH(CH₃)—CH₂—O—CH₃), C(CH₂CH₃), C-iPr, C—CH₂-cyclopropyl, C(OCH₃), C(OCH₂CH₃), C(O—(CH₂)₂O—CH₃, C(OCHF₂), C(Cl), C(C(O)CH3), C(O—CH₂CH₂—O—CH₃), C(OH));
   X₅, X₆, X₈ and X₉ are each independently nitrogen or carbon atoms;
      wherein if either one of X₅, X₆, X₈ and X₉ is nitrogen, then the respective R₇′, R₇″, R₇′″, and R₇″″ substitution is absent;
   X₁₀ is N, CH, C(R), or C═O; wherein if X₁₀ is C═O then X₁₁ is N (e.g., C(CH₂—OH), C(CH₂—CH₂—OH), C(NH—CH₂-cyclopropyl), C(COOH), C(CH₃), C(cyclopropyl), C(isopropoxy));
   X₁₁ is N or C; wherein if X₁₁ is N then X₁₀ is C═O;
   X₁₂ is S, SO₂, O, NH, N(R), N—OH, CH═CH, CH═CH(R), C(R)═CH, N═CH, N═C(R), CH═N or C(R)═N (e.g., N—CH₂—COOH, N—CH₂—CH₂—OH, N—CH₃, N—CH₂CH₃, N-iPr, N-cyclopropyl, N—CH₂-cyclopropyl);
   R₅ is H or C₁-C₅ linear or branched alkyl (e.g. methyl);
   R₆ is H, F, Cl, Br, I, OH, SH, R₈—OH, R₈—SH, —R₈—O—R₁₀ (e.g., CH₂—O—CH₃, (CH₂)₂O—CH₃ (CH₂)₃O—CH₃, (CH₂)₂O—CH(CH₃)₂), R₈—S—R₁₀ (e.g., (CH₂)₃—S—(CH₂)₂CH₃), R₈—NHC(O)—R₁₀, —O—R₈-R₁₀, R₈-(substituted or unsubstituted C₃-C₈ cycloalkyl) (e.g., CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted, saturated, unsaturated or aromatic, single, fused or spiro 3-10 membered heterocyclic ring) (e.g., (CH₂)₃-piperidine, (CH₂)₃-4-fluoro-piperidine, (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-oxa-azaspirodecane, (CH₂)₃-dimethylpyrazole, CH₂-2-oxo-methylpyrrolidine, CH₂-methyl-azetidine, CH₂-azaspiroheptane), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁) (e.g., (CH₂)₂—NH₂, (CH₂)₃—N(CH₂CH₃)₂, (CH₂)₃—N(CH(CH₃)₂)₂, (CH₂)₃-piperidine, (CH₂)₃-4-fluoro-piperidine, (CH₂)₃-4-cyano-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃)), R₉—R₈—N(R₁₀)(R₁₁) (e.g., (CH₂)₂—C(O)-piperidine), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)CH₃), C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₅ cycloalkyl (e.g., cyclopropyl, cyclobutyl, cyclohexyl, methoxycyclopropyl, methylcyclobutyl, aminomethyl-cyclobutyl, methoxycyclobutyl, 2,3-dihydro-1H-indenol), R₈-(substituted or unsubstituted C₃-C₅ cycloalkyl), substituted or unsubstituted 3-8 membered heterocyclic ring (e.g., piperidine, 1-methyl-piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), substituted or unsubstituted benzyl;
   or R₆ and R₅ are joined to for a substituted or unsubstituted 5-8 membered heterocyclic ring (e.g., azepane, piperazine, 2-(piperazin-1-yl)acetamide;
   or R₆ is represented by the structure of formula B or Bi:

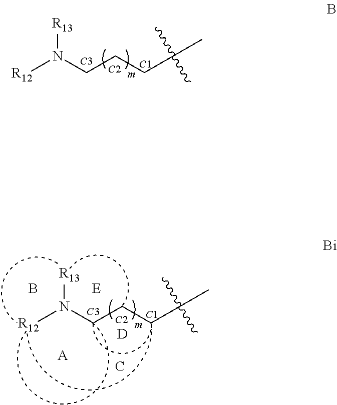

wherein
m is 0 or 1; and
   R₁₂ is R₂₀ or C₁-C₅ C(O)-alkyl, and R₁₃ is R₃₀; or
   R₁₂ and R₁₃ are both H;
   R₁₂ and R₁₃ are each independently H or substituted or unsubstituted C₁-C₅ alkyl (e.g., ethyl, trifluoroethyl);
   R₁₂ and C3 are joined to form ring A and R₁₃ is R₃₀; or
   R₁₂ and R₁₃ are joined to form ring B; or
   R₁₂ and C1 are joined to form ring C and R₁₃ is R₃₀; or
   C1 and C3 are joined to form ring D and R₁₂ and R₁₃ are each independently R₃₀; or
   R₁₃ and C2 are joined to form ring E, m is 1, and R₁₂ is R₃₀; or
   R₁₂ and R₁₃ are joined to form ring B and C1 and C3 are joined to form ring D;
   wherein
      Ring A, C and E are each independently a substituted or unsubstituted single spiro or fused 3-8 membered heterocyclic ring (e.g., A: pyrrolidine, methylpyrrolidine, ethylpyrrolidine); C: piperidine, pyrrolidine, methyl-2-oxopyrrolidine, pyran-pyrrolidine, methyl-azetidine, azabicyclooctane, 2-azabicyclo[2.1.1]hexane, 2-azaspiro[3.3]heptane; E: pyrrolidine, azetidine, ethylpyrrolidine, oxopyrrolidine, methylpiperidine);
      Ring B is a substituted or unsubstituted single, spiro or fused 3-8 membered heterocyclic ring (B: piperidine, piperidin-2-one, 4-fluoropiperidin-2-one, piperidine-4-carbonitrile, 4-fluoropiperidine, 4-fluoro-2-methylpiperidine, methyl-piperidin, fluoropiperidine, difluoropiperidine, pyrrolidine, piperazine, methylpyrrolidine, thiomorpholine 1,1-dioxide, 2-oxa-6-azaspiro[3.3]heptane, methyl-piperazine, dimethyl-pyrazole, imidazole, 2-methyl-2,5-diazabicyclo[2.2.1]heptane, hydroxymethyl-pyrrolidine, diazabicyclo[2.2.1]heptane, 6-fluoro-3-azabicyclo[3.1.1]heptane; and
      Ring D is a substituted or unsubstituted C₃-C₈ cycloalkyl (e.g., cyclobutane, cyclohexane);
R₇, R₇′, R₇″, R₇′″ and R₇″″ are each independently H, F, Cl, Br, I, OH, O—R₂₀, SH, R₈—OH, R₈—SH, —R₈—O—R₁₀, R₈—(C₃-C₅ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈-N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclohexyl), substituted or unsubstituted 3-8 membered heterocyclic ring (e.g., morpholine, pyran, oxetane, pyrrolidine, tetrahydrofuran, imidazole, piperazine, piperidine, dioxazole, 2-oxopyrrolidine), substituted or unsubstituted aryl (e.g., phenyl), substituted or unsubstituted benzyl;
or R₇′ and R₇″ are joined to form a 3-8 membered substituted or unsubstituted, saturated, unsaturated or aromatic, carbocyclic or heterocyclic ring (e.g., cyclopentyl, cyclohexyl, piperidine, tetrahydrofuran, tetrahydropyran, pyrrolidine);
or R₇″ and R₇ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
or R₇ and R₇′″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
or R₇′″ and R₇″″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
R₂₀ is represented by the following structure:

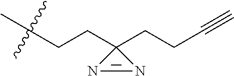 [SMILES: *CCC1(CCC#C)N=N1]

R is H, F, Cl, Br, I, OH, SH, COOH, CO(R₁₀) (e.g., C(O)CH₃), NH(R₁₀), NH—CH₂-cyclopropyl, N(R₁₀)(R₁₁), CF₃, CN, NO₂, C₁-C₅ linear or branched, substituted or unsubstituted alkyl (e.g., methyl, ethyl, iPr, CH₂-cyclopropyl, CH₂—OH, CH₂—CH₂—OH, CH₂—CH₂—O—CH₂—CH₂—O—CH₃, CH₂—O—CH₂—CH₂-O—CH₃), C₃-C₈ substituted or unsubstituted cycloalkyl, cyclopropyl, substituted or unsubstituted C₁-C₅ linear or branched alkoxy, (e.g., methoxy, ethoxy, O—(CH₂CH₃), OCHF₂, O—(CH₂)₂O—CH₃, isopropoxy, O—(CH₂)-cyclopropyl, O—CH₂-methylcyclobutyl, O—CH₂-3-methyloxetane, O—CH(CH₃)—CH₂—O—CH₃) C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, O—R₈-R₁₀ (e.g. O—(CH₂)₂O—CH₃, O—(CH₂CH₃), O—(CH₂)-cyclopropyl), —R₈—R₁₀ (e.g., (CH₂)-cyclopropyl, (CH₂)₂O—CH₃, (CH₂)—OH, (CH₂)₂—OH, (CH₂)—COOH), substituted or unsubstituted aryl (e.g., phenyl), substituted or unsubstituted heteroaryl (e.g., pyridine (2, 3, and 4-pyridine);
R₃₀ is H, R₂₀, F, Cl, Br, I, OH, SH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);
R₅₀ is H, R₂₀, F, Cl, Br, I, OH, SH, N(R)₂, NH(R₁₀), N(R₁₀)(R₁₁), CF₃, CN, NO₂, C₁-C₅ linear or branched, substituted or unsubstituted alkyl C₁-C₅ linear or branched alkoxy, C₁-C₅ linear or branched haloalkyl, —R₈—R₁₀ (e.g., (CH₂)₂O—CH₃), substituted or unsubstituted aryl (e.g., phenyl), substituted or unsubstituted heteroaryl (e.g., pyridine (2, 3, and 4-pyridine);
R is H, F, Cl, Br, I, OH, SH, COOH, CO(R₁₀) (e.g., C(O)CH₃), NH(R₁₀), NH—CH₂-cyclopropyl, N(R₁₀)(R₁₁), CF₃, CN, NO₂, C₁-C₅ linear or branched, substituted or unsubstituted alkyl (e.g., methyl, ethyl, iPr, CH₂-cyclopropyl, CH₂—OH, CH₂—CH₂—OH, CH₂—CH₂—O—CH₂—CH₂—O—CH₃, CH₂—O—CH₂—CH₂-O—CH₃), C₃-C₈ substituted or unsubstituted cycloalkyl, cyclopropyl, substituted or unsubstituted C₁-C₅ linear or branched alkoxy, (e.g., methoxy, ethoxy, O—(CH₂CH₃), OCHF₂, O—(CH₂)₂O—CH₃, isopropoxy, O—(CH₂)-cyclopropyl, O—CH₂-methylcyclobutyl, O—CH₂-3-methyloxetane, O—CH(CH₃)—CH₂—O—CH₃) C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, O—R₈-R₁₀ (e.g. O—(CH₂)₂O—CH₃, O—(CH₂CH₃), O—(CH₂)-cyclopropyl), —R₈—R₁₀ (e.g., (CH₂)-cyclopropyl, (CH₂)₂O—CH₃, (CH₂)—OH, (CH₂)₂—OH, (CH₂)—COOH), substituted or unsubstituted aryl (e.g., phenyl), substituted or unsubstituted heteroaryl (e.g., pyridine (2, 3, and 4-pyridine);
each R₈ is independently [CH₂]ₚ
   wherein p is between 1 and 10 (e.g., 1, 2);
R₉ is [CH]_{q}, [C]_{q}
   wherein q is between 2 and 10;
R₁₀ and R₁₁ are each independently H, OH, COOH, C₁-C₅ substituted or unsubstituted linear or branched alkyl (e.g., methyl, ethyl, CH₂-cyclopropyl, CH₂—CH₂—O—CH₃), C₃-C₈ substituted or unsubstituted cycloalkyl (e.g., cyclopropyl), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;
or R₁₀ and R₁₁ are joined to form a substituted or unsubstituted 3-8 membered heterocyclic ring (e.g., piperazine, piperidine),
n is an integer between 0 and 4 (e.g., 1, 2);
or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In some embodiments, at least one of R₇, R₇′, R₇″, R₇′″ and R₇″″ is not H. In some embodiments, at least two of R₇, R₇′, R₇″, R₇′″ and R₇″″ are not H. In some embodiments, at least one of R₇′, R₇″, R₇′″ and R₇″″ is not H. In some embodiments, at least two of R₇′, R₇″, R₇′″ and R₇″″ are not H. In some embodiments, at least two of R₇′, R₇″, R₇′″ and R₇″″ are different then each other. In some embodiments, at least two of R₇′, R₇″, R₇′″ and R₇″″ are not H and are different then each other.

In some embodiments, at least one of X₂, X₃, and X₄ is C(R). In some embodiments, R is not H.

In various embodiments, this invention is directed to a compound represented by the structure of formula I(j):

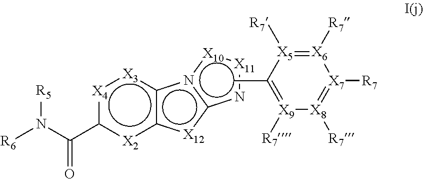

wherein
   X₂, X₃, and X₄, are each independently CH, C(R) or N (e.g., C(CH₃), C(O—CH₂-cyclopropyl), C(O—CH₂-methylcyclobutyl), C(NH—CH₂-cyclopropyl), C(isopropoxy), C(O—CH(CH₃)—CH₂—O—CH₃), C(CH₂CH₃), C-iPr, C—CH₂-cyclopropyl, C(OCH₃), C(OCH₂CH₃), C(O—(CH₂)₂O—CH₃, C(OCHF₂), C(Cl), C(C(O)CH3), C(O—CH₂CH₂—O—CH₃), C(OH));
   X₅, X₆, X₇, X₈ and X₉ are each independently nitrogen or carbon atoms;
      wherein if either one of X₅, X₆, X₇, X₈ and X₉ is nitrogen, then the respective R₇′, R₇″, R₇, R₇′″, and R₇″″ substitution is absent;
   X₁₀ is nitrogen, carbon, CH or C(R) (e.g., C(CH₂—OH), C(CH₂—CH₂—OH), C(NH—CH₂-cyclopropyl), C(COOH), C(CH₃), C(cyclopropyl), C(isopropoxy));
   X₁₁ is N or C; wherein if X₁₁ is N then X₁₀ is C═O;
   X₁₂ is S, SO₂, O, NH, N(R), N—OH, CH═CH, CH═CH(R), C(R)═CH, N═CH, N═C(R), CH═N or C(R)═N (e.g., N—CH₂—COOH, N—CH₂—CH₂—OH, N—CH₃, N—CH₂CH₃, N-iPr, N-cyclopropyl, N—CH₂-cyclopropyl);
   R₅ is H or C₁-C₅ linear or branched alkyl (e.g. methyl);
   R₆ is H, F, Cl, Br, I, OH, SH, R₈—OH, R₈—SH, —R₈—O—R₁₀ (e.g., CH₂—O—CH₃, (CH₂)₂O—CH₃ (CH₂)₃O—CH₃, (CH₂)₂O—CH(CH₃)₂), R₈—S—R₁₀ (e.g., (CH₂)₃—S—(CH₂)₂CH₃), R₈—NHC(O)—R₁₀, —O—R₈-R₁₀, R₈-(substituted or unsubstituted C₃-C₈ cycloalkyl) (e.g., CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted, saturated, unsaturated or aromatic, single, fused or spiro 3-10 membered heterocyclic ring) (e.g., (CH₂)₃-piperidine, (CH₂)₃-4-fluoro-piperidine, (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-oxa-azaspirodecane, (CH₂)₃-dimethylpyrazole, CH₂-2-oxo-methylpyrrolidine, CH₂-methyl-azetidine, CH₂-azaspiroheptane), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁) (e.g., (CH₂)₂—NH₂, (CH₂)₃—N(CH₂CH₃)₂, (CH₂)₃—N(CH(CH₃)₂)₂, (CH₂)₃-piperidine, (CH₂)₃-4-fluoro-piperidine, (CH₂)₃-4-cyano-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃)), R₉—R₈—N(R₁₀)(R₁₁) (e.g., (CH₂)₂—C(O)-piperidine), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)CH₃), C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₅ cycloalkyl (e.g., cyclopropyl, cyclobutyl, cyclohexyl, methoxycyclopropyl, methylcyclobutyl, aminomethyl-cyclobutyl, methoxycyclobutyl, 2,3-dihydro-1H-indenol), R₈-(substituted or unsubstituted C₃-C₈ cycloalkyl), substituted or unsubstituted 3-8 membered heterocyclic ring (e.g., piperidine, 1-methyl-piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), substituted or unsubstituted benzyl;
   or R₆ and R₅ are joined to form a substituted or unsubstituted saturated, unsaturated or aromatic, single, fused or spiro 5-8 membered heterocyclic ring (e.g., azepane, piperazine, 2-(piperazin-1-yl)acetamide;
   or R₆ is represented by the structure of formula B or Bi:

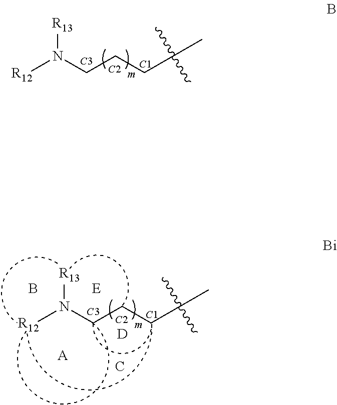

wherein
m is 0 or 1; and
   R₁₂ is R₂₀ or C₁-C₅ C(O)-alkyl, and R₁₃ is R₃₀; or
   R₁₂ and R₁₃ are both H;
   R₁₂ and R₁₃ are each independently H or substituted or unsubstituted C₁-C₅ alkyl (e.g., ethyl, trifluoroethyl);
   R₁₂ and C3 are joined to form ring A and R₁₃ is R₃₀; or
   R₁₂ and R₁₃ are joined to form ring B; or
   R₁₂ and C1 are joined to form ring C and R₁₃ is R₃₀; or
   C1 and C3 are joined to form ring D and R₁₂ and R₁₃ are each independently R₃₀; or
   R₁₃ and C2 are joined to form ring E, m is 1, and R₁₂ is R₃₀; or
   R₁₂ and R₁₃ are joined to form ring B and C1 and C3 are joined to form ring D;
   wherein
      Ring A, C and E are each independently a substituted or unsubstituted single spiro or fused 3-8 membered heterocyclic ring (e.g., A: pyrrolidine, methylpyrrolidine, ethylpyrrolidine); C: piperidine, pyrrolidine, methyl-2-oxopyrrolidine, pyran-pyrrolidine, methyl-azetidine, azabicyclooctane, 2-azabicyclo[2.1.1]hexane, 2-azaspiro[3.3]heptane; E: pyrrolidine, azetidine, ethylpyrrolidine, oxopyrrolidine, methylpiperidine);
      Ring B is a substituted or unsubstituted single, spiro or fused 3-8 membered heterocyclic ring (B: piperidine, piperidin-2-one, 4-fluoropiperidin-2-one, piperidine-4-carbonitrile, 4-fluoropiperidine, 4-fluoro-2-methylpiperidine, methyl-piperidin, fluoropiperidine, difluoropiperidine, pyrrolidine, piperazine, methylpyrrolidine, thiomorpholine 1,1-dioxide, 2-oxa-6-azaspiro[3.3]heptane, methyl-piperazine, dimethyl-pyrazole, imidazole, 2-methyl-2,5-diazabicyclo[2.2.1]heptane, hydroxymethyl-pyrrolidine, diazabicyclo[2.2.1]heptane, 6-fluoro-3-azabicyclo[3.1.1]heptane; and
      Ring D is a substituted or unsubstituted C₃-C₈ cycloalkyl (e.g., cyclobutane, cyclohexane);
R₇, R₇′, R₇″, R₇′″ and R₇″″ are each independently H, F, Cl, Br, I, OH, O—R₂₀, SH, R₈—OH, R₈—SH, —R₈—O—R₁₀, R₈—(C₃-C₅ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈-N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclohexyl), substituted or unsubstituted 3-8 membered heterocyclic ring (e.g., morpholine, pyran, oxetane, pyrrolidine, tetrahydrofuran, imidazole, piperazine, piperidine, dioxazole, 2-oxopyrrolidine), substituted or unsubstituted aryl (e.g., phenyl), substituted or unsubstituted benzyl;
or R₇′ and R₇″ are joined to form a 3-8 membered substituted or unsubstituted, saturated, unsaturated or aromatic, carbocyclic or heterocyclic ring (e.g., cyclopentyl, cyclohexyl, piperidine, tetrahydrofuran, tetrahydropyran, pyrrolidine);
or R₇″ and R₇ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
or R₇ and R₇′″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
or R₇′″ and R₇″″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
R₂₀ is represented by the following structure:

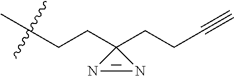 [SMILES: *CCC1(CCC#C)N=N1]

R is H, F, Cl, Br, I, OH, SH, COOH, CO(R₁₀) (e.g., C(O)CH₃), NH(R₁₀), NH—CH₂-cyclopropyl, N(R₁₀)(R₁₁), CF₃, CN, NO₂, C₁-C₅ linear or branched, substituted or unsubstituted alkyl (e.g., methyl, ethyl, iPr, CH₂-cyclopropyl, CH₂—OH, CH₂—CH₂—OH, CH₂—CH₂—O—CH₂—CH₂—O—CH₃, CH₂—O—CH₂—CH₂-O—CH₃), C₃-C₈ substituted or unsubstituted cycloalkyl, cyclopropyl, substituted or unsubstituted C₁-C₅ linear or branched alkoxy, (e.g., methoxy, ethoxy, O—(CH₂CH₃), OCHF₂, O—(CH₂)₂O—CH₃, isopropoxy, O—(CH₂)-cyclopropyl, O—CH₂-methylcyclobutyl, O—CH₂-3-methyloxetane, O—CH(CH₃)—CH₂—O—CH₃) C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, O—R₈-R₁₀ (e.g. O—(CH₂)₂O—CH₃, O—(CH₂CH₃), O—(CH₂)-cyclopropyl), —R₈—R₁₀ (e.g., (CH₂)-cyclopropyl, (CH₂)₂O—CH₃, (CH₂)—OH, (CH₂)₂—OH, (CH₂)—COOH), substituted or unsubstituted aryl (e.g., phenyl), substituted or unsubstituted heteroaryl (e.g., pyridine (2, 3, and 4-pyridine);
R₃₀ is H, R₂₀, F, Cl, Br, I, OH, SH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);
each R₅ is independently [CH₂]ₚ
   wherein p is between 1 and 10 (e.g., 1, 2);
R₉ is [CH]_{q}, [C]_{q}
   wherein q is between 2 and 10;
R₁₀ and R₁₁ are each independently H, OH, COOH, C₁-C₅ substituted or unsubstituted linear or branched alkyl (e.g., methyl, ethyl, CH₂-cyclopropyl, CH₂—CH₂—O—CH₃), C₃-C₈ substituted or unsubstituted cycloalkyl (e.g., cyclopropyl), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;
or R₁₀ and R₁₁ are joined to form a substituted or unsubstituted 3-8 membered heterocyclic ring (e.g., piperazine, piperidine),
n is an integer between 0 and 4 (e.g., 1, 2);
or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In some embodiments, at least one of R₇, R₇′, R₇″, R′″ and R₇″″ is not H. In some embodiments, at least two of R₇, R₇′, R₇″, R₇′″ and R₇″″ are not H. In some embodiments, at least one of R₇′, R₇″, R₇′″ and R₇″″ is not H. In some embodiments, at least two of R₇′, R₇″, R₇′″ and R₇″″ are not H. In some embodiments, at least two of R₇′, R₇″, R₇′″ and R₇″″ are different than each other. In some embodiments, at least two of R₇′, R₇″, R₇′″ and R₇″″ are not H and are different than each other.

In some embodiments, at least one of X₂, X₃, and X₄ is C(R). In some embodiments, X₁₁ is N. In some embodiments, X₁₂ is not S. In some embodiments, at least one of X₂, X₃, and X₄ is C(R); X₁₁ is N; or X₁₂ is not S. In some embodiments, R is not H.

In various embodiments, this invention is directed to a compound represented by the structure of formula I(k):

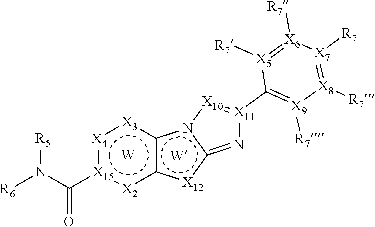

wherein
Ring W may be either aromatic or non-aromatic ring,
   wherein if ring W is aromatic then
      X₂, X₃, and X₄, are each independently CH, C(R) or N (e.g., C(CH₃), C(O—CH₂-cyclopropyl), C(O—CH₂-methylcyclobutyl), C(NH—CH₂-cyclopropyl), C(isopropoxy), C(O—CH(CH₃)—CH₂—O—CH₃), C(CH₂CH₃), C-iPr, C—CH₂-cyclopropyl, C(OCH₃), C(OCH₂CH₃), C(O—(CH₂)₂O—CH₃, C(OCHF₂), C(Cl), C(C(O)CH3), C(O—CH₂CH₂—O—CH₃), C(OH));
      X₁₅ is C;
   wherein if ring W is non-aromatic then
      X₂, X₃, and X₄, are each independently CH₂, CH(R), C(R)₂, NH, N(R), O, S, S═O or SO₂ (e.g., CH₂);
      X₁₅ is CH, C(R) or N (e.g., CH, N);
X₅, X₆, X₇, X₈ and X₉ are each independently nitrogen or carbon atoms;
   wherein if either one of X₅, X₆, X₇, X₈ and X₉ is nitrogen, then the respective R₇′, R₇″, R₇, R₇′″, and R₇″″ substitution is absent;
X₁₀ is N, CH, C(R), or C═O; wherein if X₁₀ is C═O then X₁₁ is N (e.g., C(CH₂—OH), C(CH₂—CH₂—OH), C(NH—CH₂-cyclopropyl), C(COOH), C(CH₃), C(cyclopropyl), C(isopropoxy));
X₁₁ is N or C; wherein if X₁₁ is N then X₁₀ is C═O;
Ring W′ may be either aromatic or non-aromatic ring,
   wherein if ring W′ is aromatic then
      X₁₂ is S, SO₂, O, NH, N(R), N—OH, CH═CH, CH═CH(R), C(R)═CH, N═CH, N═C(R), CH═N or C(R)═N (e.g., N—CH₂—COOH, N—CH₂—CH₂—OH, N—CH₃, N—CH₂CH₃, N-iPr, N-cyclopropyl, N—CH₂-cyclopropyl);
   wherein if ring W′ is non-aromatic then
      X₁₂ is CH═CH, CH═CH(R), C(R)═CH, OCH₂, CH₂O, SCH₂, CH₂S, CH═N, C(R)═N, N═CH, N═C(R);
R₅ is H or C₁-C₅ linear or branched alkyl (e.g. methyl);
R₆ is H, F, Cl, Br, I, OH, SH, R₈—OH, R₈—SH, —R₈—O—R₁₀ (e.g., CH₂—O—CH₃, (CH₂)₂O—CH₃ (CH₂)₃O—CH₃, (CH₂)₂O—CH(CH₃)₂), R₈—S—R₁₀ (e.g., (CH₂)₃—S—(CH₂)₂CH₃), R₈—NHC(O)—R₁₀, —O—R₈-R₁₀, R₈-(substituted or unsubstituted C₃-C₈ cycloalkyl) (e.g., CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted, saturated, unsaturated or aromatic, single, fused or spiro 3-10 membered heterocyclic ring) (e.g., (CH₂)₃-piperidine, (CH₂)₃-4-fluoro-piperidine, (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-oxa-azaspirodecane, (CH₂)₃-dimethylpyrazole, CH₂-2-oxo-methylpyrrolidine, CH₂-methyl-azetidine, CH₂-azaspiroheptane), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁) (e.g., (CH₂)₂—NH₂, (CH₂)₃—N(CH₂CH₃)₂, (CH₂)₃—N(CH(CH₃)₂)₂, (CH₂)₃-piperidine, (CH₂)₃-4-fluoro-piperidine, (CH₂)₃-4-cyano-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃)), R₉—R₈—N(R₁₀)(R₁₁) (e.g., (CH₂)₂—C(O)-piperidine), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)CH₃), C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₅ cycloalkyl (e.g., cyclopropyl, cyclobutyl, cyclohexyl, methoxycyclopropyl, methylcyclobutyl, aminomethyl-cyclobutyl, methoxycyclobutyl, 2,3-dihydro-1H-indenol), R₈-(substituted or unsubstituted C₃-C₅ cycloalkyl), substituted or unsubstituted 3-8 membered heterocyclic ring (e.g., piperidine, 1-methyl-piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), substituted or unsubstituted benzyl;
or R₆ and R₅ are joined to for a substituted or unsubstituted 5-8 membered heterocyclic ring (e.g., azepane, piperazine, 2-(piperazin-1-yl)acetamide;
or R₆ is represented by the structure of formula B or Bi:

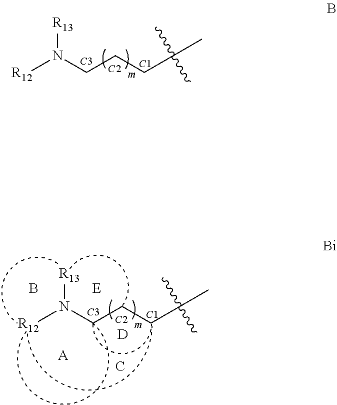

wherein
m is 0 or 1; and
   R₁₂ is R₂₀ or C₁-C₅ C(O)-alkyl, and R₁₃ is R₃₀; or
   R₁₂ and R₁₃ are both H;
   R₁₂ and R₁₃ are each independently H or substituted or unsubstituted C₁-C₅ alkyl (e.g., ethyl, trifluoroethyl);
   R₁₂ and C3 are joined to form ring A and R₁₃ is R₃₀; or
   R₁₂ and R₁₃ are joined to form ring B; or
   R₁₂ and C1 are joined to form ring C and R₁₃ is R₃₀; or
   C1 and C3 are joined to form ring D and R₁₂ and R₁₃ are each independently R₃₀; or
   R₁₃ and C2 are joined to form ring E, m is 1, and R₁₂ is R₃₀; or
   R₁₂ and R₁₃ are joined to form ring B and C1 and C3 are joined to form ring D;
   wherein
      Ring A, C and E are each independently a substituted or unsubstituted single spiro or fused 3-8 membered heterocyclic ring (e.g., A: pyrrolidine, methylpyrrolidine, ethylpyrrolidine); C: piperidine, pyrrolidine, methyl-2-oxopyrrolidine, pyran-pyrrolidine, methyl-azetidine, azabicyclooctane, 2-azabicyclo[2.1.1]hexane, 2-azaspiro[3.3]heptane; E: pyrrolidine, azetidine, ethylpyrrolidine, oxopyrrolidine, methylpiperidine);
      Ring B is a substituted or unsubstituted single, spiro or fused 3-8 membered heterocyclic ring (B: piperidine, piperidin-2-one, 4-fluoropiperidin-2-one, piperidine-4-carbonitrile, 4-fluoropiperidine, 4-fluoro-2-methylpiperidine, methyl-piperidin, fluoropiperidine, difluoropiperidine, pyrrolidine, piperazine, methylpyrrolidine, thiomorpholine 1,1-dioxide, 2-oxa-6-azaspiro[3.3]heptane, methyl-piperazine, dimethyl-pyrazole, imidazole, 2-methyl-2,5-diazabicyclo[2.2.1]heptane, hydroxymethyl-pyrrolidine, diazabicyclo[2.2.1]heptane, 6-fluoro-3-azabicyclo[3.1.1]heptane; and
      Ring D is a substituted or unsubstituted C₃-C₈ cycloalkyl (e.g., cyclobutane, cyclohexane);
R₇ is H, F, Cl, Br, I, OH, O—R₂₀, SH, R₈—OH, R₈—SH, SR₁₀, —R₈—O—R₁₀, —R₈—S—R₁₀, R₈—(C₃-C₅ cycloalkyl), CF₃, CD₃, OCD₃, CN, NO₂, —CH₂CN, —R₈CN, NH₂, NHR, N(R)₂, NH(R₁₀), N(R₁₀)(R₁₁), R₈-N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)), C(O)N(R₁₀)(R₁₁) (e.g., C(O)NH(CH₃), C(O)NH(CH₂CH₂OCH₃), C(O)NH(CH₂CH₂OH)), SO₂R, SO₂N(R₁₀)(R₁₁), CH(CF₃)(NH—R₁₀), C₁-C₅ linear or branched, substituted or unsubstituted alkyl (e.g., methylimidazole, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy, ethoxy), optionally wherein at least one methylene group (CH₂) in the alkoxy is replaced with an oxygen atom, C₁-C₅ linear or branched thioalkyl, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopropanol, cyclohexyl), substituted or unsubstituted 4-7 membered heterocyclic ring (e.g., morpholine (e.g., 2 or 3-morpholine), tetrahydrofuran, tetrahydropyran, oxetane, oxetan-3-ol, pyrrolidine, pyrrolidine-3-ol, 1-methylpyrrolidine, pyrrolidin-2-one, pyrrolidinone, imidazole, pyrazole, piperazine, piperidine, piperidine-4-ol, piperidine-4-carbonitrile, 4-fluoropiperidine, oxadiazole, triazole, 2-oxopyrrolidine, pyridine, 1-methylpyridine), R₈-(substituted or unsubstituted single, fused or spiro 3-8 membered heterocyclic ring), substituted or unsubstituted aryl, substituted or unsubstituted benzyl;
or R₇ is represented by the structure of formula A:

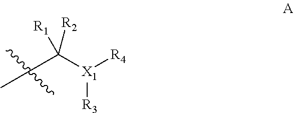

wherein
   X₁ is N or O;
   R₁ and R₂ are each independently H, F, Cl, Br, I, OH, SH, or CF₃, substituted or unsubstituted C₁-C₅ alkyl, C₁-C₅ linear or branched, or C₃-C₈ cyclic haloalkyl, substituted or unsubstituted C₁-C₅ linear or branched, or C₃-C₈ cyclic alkoxy;
   R₁ and R₂ are joined to form ═O or a C₃-C₈ carbocyclic or heterocyclic ring (e.g., cyclopropyl);
   R₃ and R₄ are each independently H, Me, substituted or unsubstituted C₁-C₅ alkyl (e.g., methoxyethylene, methylaminoethyl, aminoethyl), —R₈—O—R₁₀ (e.g., (CH₂)₂O—CH₃), R₈-N(R₁₀)(R₁₁) (e.g., (CH₂)₂—NH(CH₃)), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀;
   R₃ and R₄ are joined to form a 3-8 membered heterocyclic ring (e.g., pyrrolidine, 2-oxopyrrolidine, piperidine, morpholine, piperazine, imidazole);
   or R₂ and R₄ are joined to form a substituted or unsubstituted, saturated or unsaturated, 3-8 membered heterocyclic or carbocyclic ring (e.g., pyrrolidine, 1-methylpyrrolidine, pyrrolidin-2-one, pyridine, piperidine, tetrahydrofurane, tetrahydrothiophene, cyclopropyl, oxetane, imidazole, pyrimidine, triazole, oxadiazole, pyrazole), wherein if the ring is aromatic, then R₁ and/or R₃ are absent;
   wherein if X₁ is O then R₄ is absent;
R₇′, R₇″, R₇′″ and R₇″″ are each independently H, F, Cl, Br, I, OH, O—R₂₀, SH, R₈—OH, R₈—SH, —R₈—O—R₁₀, R₈—(C₃-C₅ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₈ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclohexyl), substituted or unsubstituted 3-8 membered heterocyclic ring (e.g., morpholine, pyran, oxetane, pyrrolidine, tetrahydrofuran, imidazole, piperazine, piperidine, dioxazole, 2-oxopyrrolidine), substituted or unsubstituted aryl (e.g., phenyl), substituted or unsubstituted benzyl;
or R₇′ and R₇″ are joined to form a 3-8 membered substituted or unsubstituted, saturated, unsaturated or aromatic, carbocyclic or heterocyclic ring (e.g., cyclopentyl, cyclohexyl, piperidine, tetrahydrofuran, tetrahydropyran, pyrrolidine);
or R₇″ and R₇ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
or R₇ and R₇′″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
or R₇′″ and R₇″″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
R₂₀ is represented by the following structure:

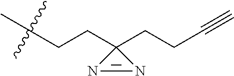 [SMILES: *CCC1(CCC#C)N=N1]

R₃₀ is H, R₂₀, F, Cl, Br, I, OH, SH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);
R is H, F, Cl, Br, I, OH, SH, COOH, CO(R₁₀) (e.g., C(O)CH₃), NH(R₁₀), NH—CH₂-cyclopropyl, N(R₁₀)(R₁₁), CF₃, CN, NO₂, C₁-C₅ linear or branched, substituted or unsubstituted alkyl (e.g., methyl, ethyl, iPr, CH₂-cyclopropyl, CH₂—OH, CH₂—CH₂—OH, CH₂—CH₂—O—CH₂—CH₂—O—CH₃, CH₂—O—CH₂—CH₂-O—CH₃), C₃-C₈ substituted or unsubstituted cycloalkyl, cyclopropyl, substituted or unsubstituted C₁-C₅ linear or branched alkoxy, (e.g., methoxy, ethoxy, O—(CH₂CH₃), OCHF₂, O—(CH₂)₂O—CH₃, isopropoxy, O—(CH₂)-cyclopropyl, O—CH₂-methylcyclobutyl, O—CH₂-3-methyloxetane, O—CH(CH₃)—CH₂—O—CH₃) C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, O—R₈-R₁₀ (e.g. O—(CH₂)₂O—CH₃, O—(CH₂CH₃), O—(CH₂)-cyclopropyl), —R₈—R₁₀ (e.g., (CH₂)-cyclopropyl, (CH₂)₂O—CH₃, (CH₂)—OH, (CH₂)₂—OH, (CH₂)—COOH), substituted or unsubstituted aryl (e.g., phenyl), substituted or unsubstituted heteroaryl (e.g., pyridine (2, 3, and 4-pyridine);
each R₈ is independently [CH₂]ₚ
   wherein p is between 1 and 10 (e.g., 1, 2);
R₉ is [CH]_{q}, [C]_{q}
   wherein q is between 2 and 10;
R₁₀ and R₁₁ are each independently H, OH, COOH, C₁-C₅ substituted or unsubstituted linear or branched alkyl (e.g., methyl, ethyl, CH₂-cyclopropyl, CH₂—CH₂—O—CH₃), C₃-C₈ substituted or unsubstituted cycloalkyl (e.g., cyclopropyl), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;
or R₁₀ and R₁₁ are joined to form a substituted or unsubstituted 3-8 membered heterocyclic ring (e.g., piperazine, piperidine),
n is an integer between 0 and 4 (e.g., 1, 2);
or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In some embodiments, at least one of R₇, R₇′, R₇″, R₇′″ and R₇″″ is not H. In some embodiments, at least two of R₇, R₇′, R₇″, R₇′″ and R₇″″ are not H. In some embodiments, at least one of R₇′, R₇″, R₇′″ and R₇″″ is not H. In some embodiments, at least two of R₇′, R₇″, R₇′″ and R₇″″ are not H. In some embodiments, at least two of R₇′, R₇″, R₇′″ and R₇″″ are different than each other. In some embodiments, at least two of R₇′, R₇″, R₇′″ and R₇″″ are not H and are different than each other.

In some embodiments, Ring W is aromatic and at least one of X₂, X₃, and X₄ is C(R). In some embodiments, Ring W is aromatic and X₁₁ is N. In some embodiments, Ring W is aromatic and X₁ is not S. In some embodiments, if Ring W is aromatic then at least one of X₂, X₃, and X₄ is C(R); X₁₁ is N; or X₁ is not S. In some embodiments, R is not H. In some embodiments, if both Ring W and Ring W′ are aromatic, then at least one of X₂, X₃, and X₄ is C(R); X₁₁ is N; or X₁₂ is not S.

In various embodiments, this invention is directed to a compound represented by the structure of formula I(l):

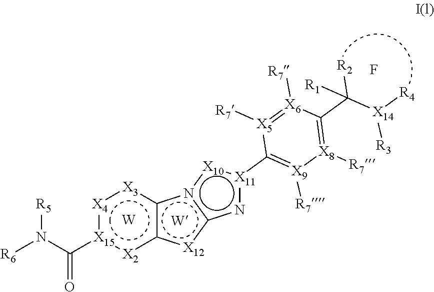

wherein
Ring W may be either aromatic or non-aromatic ring,
   wherein if ring W is aromatic then
      X₂, X₃, and X₄, are each independently CH, C(R) or N (e.g., C(CH₃), C(O—CH₂-cyclopropyl), C(O—CH₂-methylcyclobutyl), C(NH—CH₂-cyclopropyl), C(isopropoxy), CO—CH(CH₃)—CH₂—O—CH₃), C(CH₂CH₃), C-iPr, C—CH₂-cyclopropyl, C(OCH₃), C(OCH₂CH₃), C(O—(CH₂)₂O—CH₃, C(OCHF₂), C(Cl), C(C(O)CH3), C(O—CH₂CH₂—O—CH₃), C(OH));
      X₁₅ is C;
   wherein if ring W is non-aromatic then
      X₂, X₃, and X₄, are each independently CH₂, CH(R), C(R)₂, NH, N(R), O, S, S═O or SO₂ (e.g., CH₂);
X₁₅ is CH, C(R) or N (e.g., CH, N);
X₅, X₆, X₇, X₈ and X₉ are each independently nitrogen or carbon atoms;
wherein if either one of X₅, X₆, X₇, X₈ and X₉ is nitrogen, then the respective R₇′, R₇″, R₇, R₇′″, and R₇″″ substitution is absent;
X₁₀ is N, CH, C(R), or C═O; wherein if X₁₀ is C═O then X₁₁ is N (e.g., C(CH₂—OH), C(CH₂—CH₂—OH), C(NH—CH₂-cyclopropyl), C(COOH), C(CH₃), C(cyclopropyl), C(isopropoxy));
X₁₁ is N or C; wherein if X₁₁ is N then X₁₀ is C═O;
Ring W′ may be either aromatic or non-aromatic ring,
   wherein if ring W′ is aromatic then
      X₁₂ is S, SO₂, O, NH, N(R), N—OH, CH═CH, CH═CH(R), C(R)═CH, N═CH, N═C(R), CH═N or C(R)═N (e.g., N—CH₂—COOH, N—CH₂—CH₂—OH, N—CH₃, N—CH₂CH₃, N-iPr, N-cyclopropyl, N—CH₂-cyclopropyl);
   wherein if ring W′ is non-aromatic then
      X₁₂ is CH═CH, CH═CH(R), C(R)═CH, OCH₂, SCH₂, CH═N, C(R)═N, N═CH, N═C(R);
X₁₄ is S, O, N or CH, wherein if X₁₄ is CH then Ring F is not absent and if X₁₄ is S or O then R₃ is absent;
Ring F is absent or is a substituted or unsubstituted, saturated or unsaturated, 3-8 membered heterocyclic or carbocyclic ring (e.g., pyrrolidine, 1-methylpyrrolidine, pyrrolidin-2-one, pyrrolidin-3-ol, morpholine, pyridine, piperidine, tetrahydrofurane, tetrahydrothiophene, cyclopropyl, oxetane, imidazole, pyrimidine, triazole, oxadiazole, pyrazole);
R₁ and R₂ are each independently H, F, Cl, Br, I, OH, SH, or CF₃, substituted or unsubstituted C₁-C₅ alkyl, C₁-C₅ linear or branched, or C₃-C₈ cyclic haloalkyl, substituted or unsubstituted C₁-C₅ linear or branched, or C₃-C₈ cyclic alkoxy;
or R₁ and R₂ are joined to form a C═O, or a 3-8 membered carbocyclic or heterocyclic ring (e.g., cyclopropyl);
or R₂ and R₄ are joined to form Ring F as defined above (e.g., pyrrolidine, pyrrolidin-2-one, pyrrolidin-3-ol, morpholine, pyridine, pyrimidine, triazole, oxadiazole, pyrazole), wherein if Ring F is aromatic, then R₁ and/or R₃ are absent;
R₃ and R₄ are each independently H, Me, substituted or unsubstituted C₁-C₅ alkyl (e.g., methoxyethylene, methylaminoethyl, aminoethyl), —R₈—O—R₁₀ (e.g., (CH₂)₂O—CH₃), R₈—N(R₁₀)(R₁₁) (e.g., (CH₂)₂—NH(CH₃)), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀;
or R₃ and R₄ are joined to form a 3-8 membered heterocyclic ring (e.g., pyrrolidine, pyrrolidone, 2-oxopyrrolidine, piperidine, morpholine, piperazine, imidazole);
wherein if X₁₄ is S or O then R₃ is absent;
R₅ is H or C₁-C₅ linear or branched alkyl (e.g. methyl);
R₆ is H, F, Cl, Br, I, OH, SH, R₈—OH, R₈—SH, —R₈—O—R₁₀ (e.g., CH₂—O—CH₃, (CH₂)₂O—CH₃ (CH₂)₃O—CH₃, (CH₂)₂O—CH(CH₃)₂), R₈—S—R₁₀ (e.g., (CH₂)₃—S—(CH₂)₂CH₃), R₈—NHC(O)—R₁₀, —O—R₈-R₁₀, R₈-(substituted or unsubstituted C₃-C₈ cycloalkyl) (e.g., CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted, saturated, unsaturated or aromatic, single, fused or spiro 3-10 membered heterocyclic ring) (e.g., (CH₂)₃-piperidine, (CH₂)₃-4-fluoro-piperidine, (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-oxa-azaspirodecane, (CH₂)₃-dimethylpyrazole, CH₂-2-oxo-methylpyrrolidine, CH₂-methyl-azetidine, CH₂-azaspiroheptane), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁) (e.g., (CH₂)₂—NH₂, (CH₂)₃—N(CH₂CH₃)₂, (CH₂)₃—N(CH(CH₃)₂)₂, (CH₂)₃-piperidine, (CH₂)₃-4-fluoro-piperidine, (CH₂)₃-4-cyano-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃)), R₉—R₈—N(R₁₀)(R₁₁) (e.g., (CH₂)₂—C(O)-piperidine), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)CH₃), C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₅ cycloalkyl (e.g., cyclopropyl, cyclobutyl, cyclohexyl, methoxycyclopropyl, methylcyclobutyl, aminomethyl-cyclobutyl, methoxycyclobutyl, 2,3-dihydro-1H-indenol), R₈-(substituted or unsubstituted C₃-C₈ cycloalkyl), substituted or unsubstituted 3-8 membered heterocyclic ring (e.g., piperidine, 1-methyl-piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), substituted or unsubstituted benzyl;
or R₆ and R₅ are joined to for a substituted or unsubstituted 5-8 membered heterocyclic ring (e.g., azepane, piperazine, 2-(piperazin-1-yl)acetamide;
or R₆ is represented by the structure of formula B or Bi:

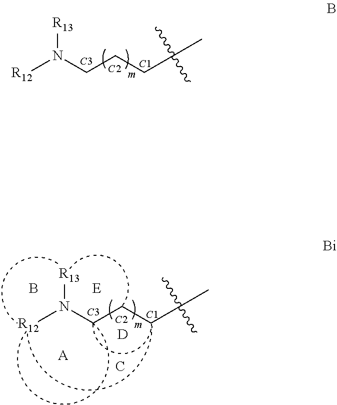

wherein
m is 0 or 1; and
   R₁₂ is R₂₀ or C₁-C₅ C(O)-alkyl, and R₁₃ is R₃₀; or
   R₁₂ and R₁₃ are both H;
   R₁₂ and R₁₃ are each independently H or substituted or unsubstituted C₁-C₅ alkyl (e.g., ethyl, trifluoroethyl);
   R₁₂ and C3 are joined to form ring A and R₁₃ is R₃₀; or
   R₁₂ and R₁₃ are joined to form ring B; or
   R₁₂ and C1 are joined to form ring C and R₁₃ is R₃₀; or
   C1 and C3 are joined to form ring D and R₁₂ and R₁₃ are each independently R₃₀; or
   R₁₃ and C2 are joined to form ring E, m is 1, and R₁₂ is R₃₀; or
   R₁₂ and R₁₃ are joined to form ring B and C1 and C3 are joined to form ring D;
   wherein
      Ring A, C and E are each independently a substituted or unsubstituted single spiro or fused 3-8 membered heterocyclic ring (e.g., A: pyrrolidine, methylpyrrolidine, ethylpyrrolidine); C: piperidine, pyrrolidine, methyl-2-oxopyrrolidine, pyran-pyrrolidine, methyl-azetidine, azabicyclooctane, 2-azabicyclo[2.1.1]hexane, 2-azaspiro[3.3]heptane; E: pyrrolidine, azetidine, ethylpyrrolidine, oxopyrrolidine, methylpiperidine);
      Ring B is a substituted or unsubstituted single, spiro or fused 3-8 membered heterocyclic ring (B: piperidine, piperidin-2-one, 4-fluoropiperidin-2-one, piperidine-4-carbonitrile, 4-fluoropiperidine, 4-fluoro-2-methylpiperidine, methyl-piperidin, fluoropiperidine, difluoropiperidine, pyrrolidine, piperazine, methylpyrrolidine, thiomorpholine 1,1-dioxide, 2-oxa-6-azaspiro[3.3]heptane, methyl-piperazine, dimethyl-pyrazole, imidazole, 2-methyl-2,5-diazabicyclo[2.2.1]heptane, hydroxymethyl-pyrrolidine, diazabicyclo[2.2.1]heptane, 6-fluoro-3-azabicyclo[3.1.1]heptane; and
      Ring D is a substituted or unsubstituted C₃-C₈ cycloalkyl (e.g., cyclobutane, cyclohexane);
R₇′, R₇″, R₇′ and R₇″″ are each independently H, F, Cl, Br, I, OH, O—R₂₀, SH, R₈—OH, R₈—SH, —R₈—O—R₁₀, R₈—(C₃-C₅ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₈ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclohexyl), substituted or unsubstituted 3-8 membered heterocyclic ring (e.g., morpholine, pyran, oxetane, pyrrolidine, tetrahydrofuran, imidazole, piperazine, piperidine, dioxazole, 2-oxopyrrolidine), substituted or unsubstituted aryl (e.g., phenyl), substituted or unsubstituted benzyl;
or R₇′ and R₇″ are joined to form a 3-8 membered substituted or unsubstituted, saturated, unsaturated or aromatic, carbocyclic or heterocyclic ring (e.g., cyclopentyl, cyclohexyl, piperidine, tetrahydrofuran, tetrahydropyran, pyrrolidine);
or R₇′″ and R₇″″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
R₂₀ is represented by the following structure:

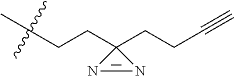 [SMILES: *CCC1(CCC#C)N=N1]

R is H, F, Cl, Br, I, OH, SH, COOH, CO(R₁₀) (e.g., C(O)CH₃), NH(R₁₀), NH—CH₂-cyclopropyl, N(R₁₀)(R₁₁), CF₃, CN, NO₂, C₁-C₅ linear or branched, substituted or unsubstituted alkyl (e.g., methyl, ethyl, iPr, CH₂-cyclopropyl, CH₂—OH, CH₂—CH₂—OH, CH₂—CH₂—O—CH₂—CH₂—O—CH₃, CH₂—O—CH₂—CH₂-O—CH₃), C₃-C₈ substituted or unsubstituted cycloalkyl, cyclopropyl, substituted or unsubstituted C₁-C₅ linear or branched alkoxy, (e.g., methoxy, ethoxy, O—(CH₂CH₃), OCHF₂, O—(CH₂)₂O—CH₃, isopropoxy, O—(CH₂)-cyclopropyl, O—CH₂-methylcyclobutyl, O—CH₂-3-methyloxetane, O—CH(CH₃)—CH₂—O—CH₃) C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, O—R₈-R₁₀ (e.g. O—(CH₂)₂O—CH₃, O—(CH₂CH₃), O—(CH₂)-cyclopropyl), —R₈—R₁₀ (e.g., (CH₂)-cyclopropyl, (CH₂)₂O—CH₃, (CH₂)—OH, (CH₂)₂—OH, (CH₂)—COOH), substituted or unsubstituted aryl (e.g., phenyl), substituted or unsubstituted heteroaryl (e.g., pyridine (2, 3, and 4-pyridine);
R₃₀ is H, R₂₀, F, Cl, Br, I, OH, SH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);
each R₈ is independently [CH₂]ₚ
   wherein p is between 1 and 10 (e.g., 1, 2);
R₉ is [CH]_{q}, [C]_{q}
   wherein q is between 2 and 10;
R₁₀ and R₁₁ are each independently H, OH, COOH, C₁-C₅ substituted or unsubstituted linear or branched alkyl (e.g., methyl, ethyl, CH₂-cyclopropyl, CH₂—CH₂—O—CH₃), C₃-C₈ substituted or unsubstituted cycloalkyl (e.g., cyclopropyl), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;
or R₁₀ and Rn are joined to form a substituted or unsubstituted 3-8 membered heterocyclic ring (e.g., piperazine, piperidine),
n is an integer between 0 and 4 (e.g., 1, 2);
or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In some embodiments, at least one of R₇′, R₇″, R₇′″ and R₇″″ is not H. In some embodiments, at least two of R₇′, R₇″, R₇′″ and R₇″″ are not H. In some embodiments, at least two of R₇′, R₇″, R₇′″ and R₇″″ are different then each other. In some embodiments, at least two of R₇′, R₇″, R₇′″ and R₇″″ are not H and are different then each other.

In some embodiments, Ring W is aromatic and at least one of X₂, X₃, and X₄ is C(R). In some embodiments, Ring W is aromatic and X₁₁ is N. In some embodiments, Ring W is aromatic and X₁ is not S. In some embodiments, if Ring W is aromatic then at least one of X₂, X₃, and X₄ is C(R); X₁₁ is N; or X₂ is not S. In some embodiments, if both Ring W and Ring W′ are aromatic, then at least one of X₂, X₃, and X₄ is C(R); X₁₁ is N; or X₁₂ is not S. In some embodiments, R is not H.

In some embodiments, if R₁ and R₂ are joined to form a C═O, then at least one of X₂, X₃, X₄, and X₁₀ is not CH;

In various embodiments, this invention is directed to a compound represented by the structure of formula I(m):

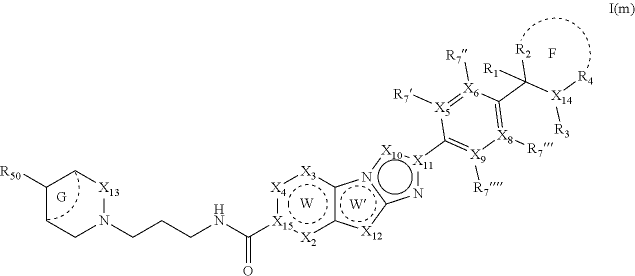

wherein
Ring W may be either aromatic or non-aromatic ring,
   wherein if ring W is aromatic then
      X₂, X₃, and X₄, are each independently CH, C(R) or N (e.g., C(CH₃), C(O—CH₂-cyclopropyl), C(O—CH₂-methylcyclobutyl), C(NH—CH₂-cyclopropyl), C(isopropoxy), C(O—CH(CH₃)—CH₂—O—CH₃), C(CH₂CH₃), C-iPr, C—CH₂-cyclopropyl, C(OCH₃), C(OCH₂CH₃), C(O—(CH₂)₂O—CH₃, C(OCHF₂), C(Cl), C(C(O)CH3), C(O—CH₂CH₂—O—CH₃), C(OH));
      X₁₅ is C;
   wherein if ring W is non-aromatic then
      X₂, X₃, and X₄, are each independently CH₂, CH(R), C(R)₂, NH, N(R), O, S, S═O or SO₂ (e.g., CH₂);
      X₁₅ is CH, C(R) or N (e.g., CH, N);
X₅, X₆, X₇, X₈ and X₉ are each independently nitrogen or carbon atoms;
   wherein if either one of X₅, X₆, X₈ and X₉ is nitrogen, then the respective R₇′, R₇″, R₇′″, and R₇″″ substitution is absent;
X₁₀ is N, CH, C(R), or C═O; wherein if X₁₀ is C═O then X₁₁ is N (e.g., C(CH₂—OH), C(CH₂—CH₂—OH), C(NH—CH₂-cyclopropyl), C(COOH), C(CH₃), C(cyclopropyl), C(isopropoxy));
X₁₁ is N or C; wherein if X₁₁ is N then X₁₀ is C═O;
Ring W′ may be either aromatic or non-aromatic ring,
   wherein if ring W′ is aromatic then
      X₁₂ is S, SO₂, O, NH, N(R), N—OH, CH═CH, CH═CH(R), C(R)═CH, N═CH, N═C(R), CH═N or C(R)═N (e.g., N—CH₂—COOH, N—CH₂—CH₂—OH, N—CH₃, N—CH₂CH₃, N-iPr, N-cyclopropyl, N—CH₂-cyclopropyl);
   wherein if ring W′ is non-aromatic then
      X₁₂ is CH═CH, CH═CH(R), C(R)═CH, OCH₂, SCH₂, CH═N, C(R)═N, N═CH, N═C(R);
X₁₃ is CH₂, CH(R) (e.g., CH—CH₃), C(R)₂, or C═O;
X₁₄ is S, O, N or CH, wherein if X₁₄ is CH then Ring F is not absent and if X₁₄ is S or O then R₃ is absent;
Ring G is absent or is a substituted or unsubstituted 3-8 membered carbocyclic or heterocyclic ring (e.g., cyclobutane, cyclopentane, cyclohexane);
Ring F is absent or is a substituted or unsubstituted, saturated or unsaturated, 3-8 membered heterocyclic or carbocyclic ring (e.g., pyrrolidine, 1-methylpyrrolidine, pyrrolidin-2-one, pyrrolidin-3-ol, morpholine, pyridine, piperidine, tetrahydrofurane, tetrahydrothiophene, cyclopropyl, oxetane, imidazole, pyrimidine, triazole, oxadiazole, pyrazole);
R₁ and R₂ are each independently H, F, Cl, Br, I, OH, SH, or CF₃, substituted or unsubstituted C₁-C₅ alkyl, C₁-C₅ linear or branched, or C₃-C₅ cyclic haloalkyl, substituted or unsubstituted C₁-C₅ linear or branched, or C₃-C₅ cyclic alkoxy;
or R₁ and R₂ are joined to form a C═O, or a 3-8 membered carbocyclic or heterocyclic ring (e.g., cyclopropyl);
or R₂ and R₄ are joined to form Ring F as defined above (e.g., pyrrolidine, pyrrolidin-2-one, pyrrolidin-3-ol, morpholine, pyridine, pyrimidine, triazole, oxadiazole, pyrazole), wherein if Ring F is aromatic, then R₁ and/or R₃ are absent;
R₃ and R₄ are each independently H, Me, substituted or unsubstituted C₁-C₅ alkyl (e.g., methoxyethylene, methylaminoethyl, aminoethyl), —R₈—O—R₁₀ (e.g., (CH₂)₂O—CH₃), R₈—N(R₁₀)(R₁₁) (e.g., (CH₂)₂—NH(CH₃)), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀;
or R₃ and R₄ are joined to form a 3-8 membered heterocyclic ring (e.g., pyrrolidine, pyrrolidone, 2-oxopyrrolidine, piperidine, morpholine, piperazine, imidazole);
R₇′, R₇″, R₇′″ and R₇″″ are each independently H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₈ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclohexyl), substituted or unsubstituted 3-8 membered heterocyclic ring (e.g., morpholine, pyran, oxetane, pyrrolidine, tetrahydrofuran, imidazole, piperazine, piperidine, dioxazole, 2-oxopyrrolidine), substituted or unsubstituted aryl (e.g., phenyl), substituted or unsubstituted benzyl;
or R₇′ and R₇″ are joined to form a 3-8 membered substituted or unsubstituted, saturated, unsaturated or aromatic, carbocyclic or heterocyclic ring (e.g., cyclopentyl, cyclohexyl, piperidine, tetrahydrofuran, tetrahydropyran, pyrrolidine);
or R₇′″ and R₇″″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
R₂₀ is represented by the following structure:

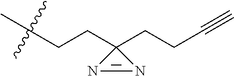 [SMILES: *CCC1(CCC#C)N=N1]

R₃₀ is H, R₂₀, F, Cl, Br, I, OH, SH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);
R₅₀ is H, R₂₀, F, Cl, Br, I, OH, SH, N(R)₂, NH(R₁₀), N(R₁₀)(R₁₁), CF₃, CN, NO₂, C₁-C₅ linear or branched, substituted or unsubstituted alkyl C₁-C₅ linear or branched alkoxy, C₁-C₅ linear or branched haloalkyl, —R₈—R₁₀ (e.g., (CH₂)₂O—CH₃), substituted or unsubstituted aryl (e.g., phenyl), substituted or unsubstituted heteroaryl (e.g., pyridine (2, 3, and 4-pyridine);
R is H, F, Cl, Br, I, OH, SH, COOH, CO(R₁₀) (e.g., C(O)CH₃), NH(R₁₀), NH—CH₂-cyclopropyl, N(R₁₀)(R₁₁), CF₃, CN, NO₂, C₁-C₅ linear or branched, substituted or unsubstituted alkyl (e.g., methyl, ethyl, iPr, CH₂-cyclopropyl, CH₂—OH, CH₂—CH₂—OH, CH₂—CH₂—O—CH₂—CH₂—O—CH₃, CH₂—O—CH₂—CH₂-O—CH₃), C₃-C₈ substituted or unsubstituted cycloalkyl, cyclopropyl, substituted or unsubstituted C₁-C₅ linear or branched alkoxy, (e.g., methoxy, ethoxy, O—(CH₂CH₃), OCHF₂, O—(CH₂)₂O—CH₃, isopropoxy, O—(CH₂)-cyclopropyl, O—CH₂-methylcyclobutyl, O—CH₂-3-methyloxetane, O—CH(CH₃)—CH₂—O—CH₃) C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, O—R₈-R₁₀ (e.g. O—(CH₂)₂O—CH₃, O—(CH₂CH₃), O—(CH₂)-cyclopropyl), —R₈—R₁₀ (e.g., (CH₂)-cyclopropyl, (CH₂)₂O—CH₃, (CH₂)—OH, (CH₂)₂—OH, (CH₂)—COOH), substituted or unsubstituted aryl (e.g., phenyl), substituted or unsubstituted heteroaryl (e.g., pyridine (2, 3, and 4-pyridine);
each R₈ is independently [CH₂]ₚ
   wherein p is between 1 and 10 (e.g., 1, 2);
R₉ is [CH]_{q}, [C]_{q}
   wherein q is between 2 and 10;
R₁₀ and R₁₁ are each independently H, OH, COOH, C₁-C₅ substituted or unsubstituted linear or branched alkyl (e.g., methyl, ethyl, CH₂-cyclopropyl, CH₂—CH₂—O—CH₃), C₃-C₈ substituted or unsubstituted cycloalkyl (e.g., cyclopropyl), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;
or R₁₀ and R₁₁ are joined to form a substituted or unsubstituted 3-8 membered heterocyclic ring (e.g., piperazine, piperidine),
n is an integer between 0 and 4 (e.g., 1, 2);
or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In some embodiments, at least one of R₇′, R₇″, R₇′″ and R₇″″ is not H. In some embodiments, at least two of R₇′, R₇″, R₇′″ and R₇″″ are not H. In some embodiments, at least two of R₇′, R₇″, R₇′″ and R₇″″ are different then each other. In some embodiments, at least two of R₇′, R₇″, R₇′″ and R₇″″ are not H and are different then each other.

In some embodiments, Ring W is aromatic and at least one of X₂, X₃, and X₄ is C(R). In some embodiments, Ring W is aromatic and X₁₁ is N. In some embodiments, Ring W is aromatic and X₂ is not S. In some embodiments, if Ring W is aromatic then at least one of X₂, X₃, and X₄ is C(R); X₁₁ is N; or X₁ is not S. In some embodiments, if both Ring W and Ring W′ are aromatic, then at least one of X₂, X₃, and X₄ is C(R); X₁₁ is N; or X₁₂ is not S. In some embodiments, R is not H.

In various embodiments, this invention is directed to a compound represented by the structure of formula I(n):

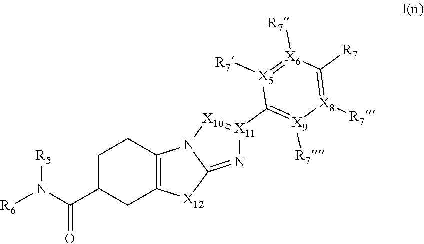

wherein
X₅, X₆, X₇, X₈ and X₉ are each independently nitrogen or carbon atoms;
   wherein if either one of X₅, X₆, X₈ and X₉ is nitrogen, then the respective R₇′, R₇″, R₇′″, and R₇″″ substitution is absent;
   X₁₀ is N, CH, C(R), or C═O; wherein if X₁₀ is C═O then X₁₁ is N (e.g., C(CH₂—OH), C(CH₂—CH₂—OH), C(NH—CH₂-cyclopropyl), C(COOH), C(CH₃), C(cyclopropyl), C(isopropoxy));
   X₁₁ is N or C; wherein if X₁₁ is N then X₁₀ is C═O;
   X₁₂ is S, SO₂, O, NH, N(R), N—OH, CH═CH, CH═CH(R), C(R)═CH, N═CH, N═C(R), CH═N or C(R)═N (e.g., N—CH₂—COOH, N—CH₂—CH₂—OH, N—CH₃, N—CH₂CH₃, N-iPr, N-cyclopropyl, N—CH₂-cyclopropyl);
   R₅ is H or C₁-C₅ linear or branched alkyl (e.g. methyl);
   R₆ is H, F, Cl, Br, I, OH, SH, R₈—OH, R₈—SH, —R₈—O—R₁₀ (e.g., CH₂—O—CH₃, (CH₂)₂O—CH₃ (CH₂)₃O—CH₃, (CH₂)₂O—CH(CH₃)₂), R₈—S—R₁₀ (e.g., (CH₂)₃—S—(CH₂)₂CH₃), R₈—NHC(O)—R₁₀, —O—R₈-R₁₀, R₈-(substituted or unsubstituted C₃-C₈ cycloalkyl) (e.g., CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted, saturated, unsaturated or aromatic, single, fused or spiro 3-10 membered heterocyclic ring) (e.g., (CH₂)₃-piperidine, (CH₂)₃-4-fluoro-piperidine, (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-oxa-azaspirodecane, (CH₂)₃-dimethylpyrazole, CH₂-2-oxo-methylpyrrolidine, CH₂-methyl-azetidine, CH₂-azaspiroheptane), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁) (e.g., (CH₂)₂—NH₂, (CH₂)₃-N(CH₂CH₃)₂, (CH₂)₃—N(CH(CH₃)₂)₂, (CH₂)₃-piperidine, (CH₂)₃-4-fluoro-piperidine, (CH₂)₃-4-cyano-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃)), R₉—R₈—N(R₁₀)(R₁₁) (e.g., (CH₂)₂—C(O)-piperidine), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)CH₃), C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclobutyl, cyclohexyl, methoxycyclopropyl, methylcyclobutyl, aminomethyl-cyclobutyl, methoxycyclobutyl, 2,3-dihydro-1H-indenol), R₈-(substituted or unsubstituted C₃-C₅ cycloalkyl), substituted or unsubstituted 3-8 membered heterocyclic ring (e.g., piperidine, 1-methyl-piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), substituted or unsubstituted benzyl;
or R₆ and R₅ are joined to for a substituted or unsubstituted 5-8 membered heterocyclic ring (e.g., azepane, piperazine, 2-(piperazin-1-yl)acetamide;
or R₆ is represented by the structure of formula B or Bi:

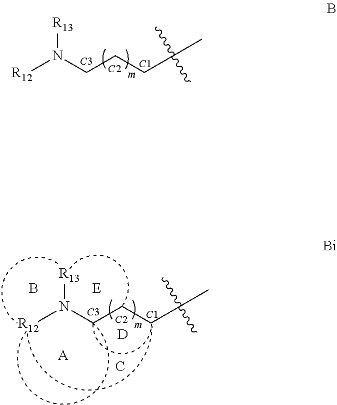

wherein
m is 0 or 1; and
   R₁₂ is R₂₀ or C₁-C₅ C(O)-alkyl, and R₁₃ is R₃₀; or
   R₁₂ and R₁₃ are both H;
   R₁₂ and R₁₃ are each independently H or substituted or unsubstituted C₁-C₅ alkyl (e.g., ethyl, trifluoroethyl);
   R₁₂ and C3 are joined to form ring A and R₁₃ is R₃₀; or
   R₁₂ and R₁₃ are joined to form ring B; or
   R₁₂ and C1 are joined to form ring C and R₁₃ is R₃₀; or
   C1 and C3 are joined to form ring D and R₁₂ and R₁₃ are each independently R₃₀; or
   R₁₃ and C2 are joined to form ring E, m is 1, and R₁₂ is R₃₀; or
   R₁₂ and R₁₃ are joined to form ring B and C1 and C3 are joined to form ring D;
   wherein
      Ring A, C and E are each independently a substituted or unsubstituted single spiro or fused 3-8 membered heterocyclic ring (e.g., A: pyrrolidine, methylpyrrolidine, ethylpyrrolidine); C: piperidine, pyrrolidine, methyl-2-oxopyrrolidine, pyran-pyrrolidine, methyl-azetidine, azabicyclooctane, 2-azabicyclo[2.1.1]hexane, 2-azaspiro[3.3]heptane; E: pyrrolidine, azetidine, ethylpyrrolidine, oxopyrrolidine, methylpiperidine);
      Ring B is a substituted or unsubstituted single, spiro or fused 3-8 membered heterocyclic ring (B: piperidine, piperidin-2-one, 4-fluoropiperidin-2-one, piperidine-4-carbonitrile, 4-fluoropiperidine, 4-fluoro-2-methylpiperidine, methyl-piperidin, fluoropiperidine, difluoropiperidine, pyrrolidine, piperazine, methylpyrrolidine, thiomorpholine 1,1-dioxide, 2-oxa-6-azaspiro[3.3]heptane, methyl-piperazine, dimethyl-pyrazole, imidazole, 2-methyl-2,5-diazabicyclo[2.2.1]heptane, hydroxymethyl-pyrrolidine, diazabicyclo[2.2.1]heptane, 6-fluoro-3-azabicyclo[3.1.1]heptane; and
      Ring D is a substituted or unsubstituted C₃-C₈ cycloalkyl (e.g., cyclobutane, cyclohexane);
R₇, R₇′, R₇″, R₇′″ and R₇″″ are each independently H, F, Cl, Br, I, OH, O—R₂₀, SH, R₈—OH, R₈—SH, —R₈—O—R₁₀, R₈—(C₃-C₅ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈-N(R₁₀)(R₁₁), R₉—R₈—N(R₁₀)(Rui), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclohexyl), substituted or unsubstituted 3-8 membered heterocyclic ring (e.g., morpholine, pyran, oxetane, pyrrolidine, tetrahydrofuran, imidazole, piperazine, piperidine, dioxazole, 2-oxopyrrolidine), substituted or unsubstituted aryl (e.g., phenyl), substituted or unsubstituted benzyl;
or R₇′ and R₇″ are joined to form a 3-8 membered substituted or unsubstituted, saturated, unsaturated or aromatic, carbocyclic or heterocyclic ring (e.g., cyclopentyl, cyclohexyl, piperidine, tetrahydrofuran, tetrahydropyran, pyrrolidine);
or R₇″ and R₇ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
or R₇ and R₇′″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
or R₇′″ and R₇″″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
R₂₀ is represented by the following structure:

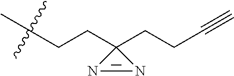 [SMILES: *CCC1(CCC#C)N=N1]

R₃₀ is H, R₂₀, F, Cl, Br, I, OH, SH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂O—CH₃), —R₈—O—R₁₀, —R₈—R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);
R is H, F, Cl, Br, I, OH, SH, COOH, CO(R₁₀) (e.g., C(O)CH₃), NH(R₁₀), NH—CH₂-cyclopropyl, N(R₁₀)(R₁₁), CF₃, CN, NO₂, C₁-C₅ linear or branched, substituted or unsubstituted alkyl (e.g., methyl, ethyl, iPr, CH₂-cyclopropyl, CH₂—OH, CH₂—CH₂—OH, CH₂—CH₂—O—CH₂—CH₂—O—CH₃, CH₂—O—CH₂—CH₂—O—CH₃), C₃-C₈ substituted or unsubstituted cycloalkyl, cyclopropyl, substituted or unsubstituted C₁-C₅ linear or branched alkoxy, (e.g., methoxy, ethoxy, O—(CH₂CH₃), OCHF₂, O—(CH₂)₂O—CH₃, isopropoxy, O—(CH₂)-cyclopropyl, O—CH₂-methylcyclobutyl, O—CH₂-3-methyloxetane, O—CH(CH₃)—CH₂—O—CH₃) C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, O—R₈-R₁₀ (e.g. O—(CH₂)₂O—CH₃, O—(CH₂CH₃), O—(CH₂)-cyclopropyl), —R₈—R₁₀ (e.g., (CH₂)-cyclopropyl, (CH₂)₂—O—CH₃, (CH₂)—OH, (CH₂)₂—OH, (CH₂)—COOH), substituted or unsubstituted aryl (e.g., phenyl), substituted or unsubstituted heteroaryl (e.g., pyridine (2, 3, and 4-pyridine);
each R₅ is independently [CH₂]ₚ
   wherein p is between 1 and 10 (e.g., 1, 2);
R₉ is [CH]_{q}, [C]_{q}
   wherein q is between 2 and 10;
R₁₀ and R₁₁ are each independently H, OH, COOH, C₁-C₅ substituted or unsubstituted linear or branched alkyl (e.g., methyl, ethyl, CH₂-cyclopropyl, CH₂—CH₂—O—CH₃), C₃-C₈ substituted or unsubstituted cycloalkyl (e.g., cyclopropyl), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;
or R₁₀ and R₁₁ are joined to form a substituted or unsubstituted 3-8 membered heterocyclic ring (e.g., piperazine, piperidine),
n is an integer between 0 and 4 (e.g., 1, 2);
or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In some embodiments, at least one of R₇′, R₇″, R₇′″ and R₇″″ is not H. In some embodiments, at least two of R₇′, R₇″, R₇′″ and R₇″″ are not H. In some embodiments, at least two of R₇′, R₇″, R₇′″ and R₇″″ are different than each other. In some embodiments, at least two of R₇′, R₇″, R′″ and R₇″″ are not H and are different than each other.

In some embodiments, at least one of X₂, X₃, and X₄ is C(R). In some embodiments, X₁₁ is N. In some embodiments, X₁₂ is not S. In some embodiments, at least one of X₂, X₃, and X₄ is C(R); X₁₁ is N; or X₁₂ is not S. In some embodiments, R is not H.

In some embodiments, X₂ of formula I and/or I(a)-I(j) is N. In other embodiments, X₂ is a CH. In other embodiments, X₂ is a C(R). In other embodiments, X₂ is C(CH₃). In other embodiments, X₂ is C(CH₂CH₃). In other embodiments, X₂ is C-iPr. In other embodiments, X₂ is C—CH₂-cyclopropyl. In other embodiments, X₂ is C(O—CH₂-cyclopropyl). In other embodiments, X₂ is C(O—CH₂-methylcyclobutyl). In other embodiments, X₂ is C(O—CH₂-3-methyloxetane). In other embodiments, X₂ is C(OCH₃) In other embodiments, X₂ is C(OCH₂CH₃) In other embodiments, X₂ is C(O—(CH₂)₂—O—CH₃. In other embodiments, X₂ is C(NH—CH₂-cyclopropyl). In other embodiments, X₂ is C(isopropoxy). In other embodiments, X₂ is C(O—CH(CH₃)—CH₂—O—CH₃). In other embodiments, X₂ is C(OCHF₂). In other embodiments, X₂ is C(Cl). In other embodiments, X₂ is C(C(O)CH₃). In other embodiments, X₂ is C(OH).

In some embodiments, Ring W of formula I(k)-I(m) is aromatic or non-aromatic. In some embodiments, if Ring W is aromatic, then X₂ is a CH. In other embodiments, if Ring W is aromatic, then X₂ is a C(R). In other embodiments, if Ring W is aromatic, then X₂ is C(CH₃). In other embodiments, if Ring W is aromatic, then X₂ is C(CH₂CH₃). In other embodiments, if Ring W is aromatic, then X₂ is C-iPr. In other embodiments, if Ring W is aromatic, then X₂ is C—CH₂-cyclopropyl. In other embodiments, if Ring W is aromatic, then X₂ is C(O—CH₂-cyclopropyl). In other embodiments, if Ring W is aromatic, then X₂ is C(O—CH₂-methylcyclobutyl). In other embodiments, if Ring W is aromatic, then X₂ is C(O—CH₂-3-methyloxetane). In other embodiments, if Ring W is aromatic, then X₂ is C(OCH₃) In other embodiments, if Ring W is aromatic, then X₂ is C(OCH₂CH₃). In other embodiments, if Ring W is aromatic, then X₂ is C(O—(CH₂)₂O—CH₃. In other embodiments, if Ring W is aromatic X₂ is C(NH—CH₂-cyclopropyl). In other embodiments, if Ring W is aromatic X₂ is C(isopropoxy). In other embodiments, if Ring W is aromatic X₂ is C(O—CH(CH₃)—CH₂—O—CH₃). In other embodiments, if Ring W is aromatic X₂ is C(OCHF₂). In other embodiments, if Ring W is aromatic X₂ is C(Cl). In other embodiments, if Ring W is aromatic X₂ is C(C(O)CH₃). In other embodiments, if Ring W is aromatic X₂ is C(OH). In some embodiments, if Ring W is non-aromatic then X₂ is CH₂. In some embodiments, if Ring W is non-aromatic then X₂ is CH(R). In some embodiments, if Ring W is non-aromatic then X₂ is C(R)₂. In some embodiments, if Ring W is non-aromatic then X₂ is NH. In some embodiments, if Ring W is non-aromatic then X₂ is N(R). In some embodiments, if Ring W is non-aromatic then X₂ is O. In some embodiments, if Ring W is non-aromatic then X₂ is S. In some embodiments, if Ring W is non-aromatic then X₂ is S═O. In some embodiments, if Ring W is non-aromatic then X₂ is SO₂.

In some embodiments, X₃ of formula I and/or I(a)-I(j) is N. In other embodiments, X₃ is a CH. In other embodiments, X₃ is a CH. In other embodiments, X₃ is a C(R). In other embodiments, X₃ is C(CH₃). In other embodiments, X₃ is C(CH₂CH₃). In other embodiments, X₃ is C-iPr. In other embodiments, X₃ is C—CH₂-cyclopropyl. In other embodiments, X₃ is C(O—CH₂-cyclopropyl). In other embodiments, X₃ is C(O—CH₂-methylcyclobutyl). In other embodiments, X₃ is C(O—CH₂-3-methyloxetane). In other embodiments, X₃ is C(OCH₃) In other embodiments, X₃ is C(OCH₂CH₃) In other embodiments, X₃ is C(O—(CH₂)₂—O—CH₃. In other embodiments, X₃ is C(NH—CH₂-cyclopropyl). In other embodiments, X₃ is C(isopropoxy). In other embodiments, X₃ is C(O—CH(CH₃)—CH₂—O—CH₃). In other embodiments, X₃ is C(OCHF₂). In other embodiments, X₃ is C(Cl). In other embodiments, X₂ is C(C(O)CH₃). In other embodiments, X₃ is C(OH).

In some embodiments, Ring W of formula I(k)-I(m) is aromatic or non-aromatic. In some embodiments, if Ring W is aromatic, then X₃ is a CH. In other embodiments, if Ring W is aromatic, then X₃ is a C(R). In other embodiments, if Ring W is aromatic, then X₃ is C(CH₃). In other embodiments, if Ring W is aromatic, then X₃ is C(CH₂CH₃). In other embodiments, if Ring W is aromatic, then X₃ is C-iPr. In other embodiments, if Ring W is aromatic, then X₃ is C—CH₂-cyclopropyl. In other embodiments, if Ring W is aromatic, then X₃ is C(O—CH₂-cyclopropyl). In other embodiments, if Ring W is aromatic, then X₃ is C(O—CH₂-methylcyclobutyl). In other embodiments, if Ring W is aromatic, then X₃ is C(O—CH₂-3-methyloxetane). In other embodiments, if Ring W is aromatic, then X₃ is C(OCH₃) In other embodiments, if Ring W is aromatic, then X₃ is C(OCH₂CH₃). In other embodiments, if Ring W is aromatic, then X₃ is C(O—(CH₂)₂O—CH₃. In other embodiments, if Ring W is aromatic X₃ is C(NH—CH₂-cyclopropyl). In other embodiments, if Ring W is aromatic X₃ is C(isopropoxy). In other embodiments, if Ring W is aromatic X₃ is C(O—CH(CH₃)—CH₂—O—CH₃). In other embodiments, if Ring W is aromatic X₃ is C(OCHF₂). In other embodiments, if Ring W is aromatic X₃ is C(Cl). In other embodiments, if Ring W is aromatic X₃ is C(C(O)CH₃). In other embodiments, if Ring W is aromatic X₃ is C(OH). In some embodiments, if Ring W is non-aromatic then X₃ is CH₂. In some embodiments, if Ring W is non-aromatic then X₃ is CH(R). In some embodiments, if Ring W is non-aromatic then X₃ is C(R)₂. In some embodiments, if Ring W is non-aromatic then X₃ is NH. In some embodiments, if Ring W is non-aromatic then X₃ is N(R). In some embodiments, if Ring W is non-aromatic then X₃ is O. In some embodiments, if Ring W is non-aromatic then X₃ is S. In some embodiments, if Ring W is non-aromatic then X₃ is S═O. In some embodiments, if Ring W is non-aromatic then X₃ is SO₂.

In some embodiments, X₄ of formula I and/or I(a)-I(j) is N. In other embodiments, X₄ is a CH. In other embodiments, X₄ is a C(R). In other embodiments, X₄ is C(CH₃). In other embodiments, X₄ is C(CH₂CH₃). In other embodiments, X₄ is C-iPr. In other embodiments, X₄ is C—CH₂-cyclopropyl. In other embodiments, X₄ is C(O—CH₂-cyclopropyl). In other embodiments, X₄ is C(O—CH₂-methylcyclobutyl). In other embodiments, X₄ is C(O—CH₂-3-methyloxetane). In other embodiments, X₄ is C(OCH₃) In other embodiments, X₄ is C(OCH₂CH₃) In other embodiments, X₄ is C(O—(CH₂)₂O—CH₃. In other embodiments, X₄ is C(NH—CH₂-cyclopropyl). In other embodiments, X₄ is C(isopropoxy). In other embodiments, X₄ is C(O—CH(CH₃)—CH₂—O—CH₃). In other embodiments, X₄ is C(OCHF₂). In other embodiments, X₄ is C(Cl). In other embodiments, X₄ is C(C(O)CH₃). In other embodiments, X₄ is C(OH).

In some embodiments, Ring W of formula I(k)-I(m) is aromatic or non-aromatic. In some embodiments, if Ring W is aromatic, then X₄ is a CH. In other embodiments, if Ring W is aromatic, then X₄ is a C(R). In other embodiments, if Ring W is aromatic, then X₄ is C(CH₃). In other embodiments, if Ring W is aromatic, then X₄ is C(CH₂CH₃). In other embodiments, if Ring W is aromatic, then X₄ is C-iPr. In other embodiments, if Ring W is aromatic, then X₄ is C—CH₂-cyclopropyl. In other embodiments, if Ring W is aromatic, then X₄ is C(O—CH₂-cyclopropyl). In other embodiments, if Ring W is aromatic, then X₄ is C(O—CH₂-methylcyclobutyl). In other embodiments, if Ring W is aromatic, then X₄ is C(O—CH₂-3-methyloxetane). In other embodiments, if Ring W is aromatic, then X₄ is C(OCH₃) In other embodiments, if Ring W is aromatic, then X₄ is C(OCH₂CH₃). In other embodiments, if Ring W is aromatic, then X₄ is C(O—(CH₂)₂O—CH₃. In other embodiments, if Ring W is aromatic X₄ is C(NH—CH₂-cyclopropyl). In other embodiments, if Ring W is aromatic X₄ is C(isopropoxy). In other embodiments, if Ring W is aromatic X₄ is C(O—CH(CH₃)—CH₂—O—CH₃). In other embodiments, if Ring W is aromatic X₄ is C(OCHF₂). In other embodiments, if Ring W is aromatic X₄ is C(Cl). In other embodiments, if Ring W is aromatic X₄ is C(C(O)CH₃). In other embodiments, if Ring W is aromatic X₄ is C(OH). In some embodiments, if Ring W is non-aromatic then X₄ is CH₂. In some embodiments, if Ring W is non-aromatic then X₄ is CH(R). In some embodiments, if Ring W is non-aromatic then X₄ is C(R)₂. In some embodiments, if Ring W is non-aromatic then X₄ is NH. In some embodiments, if Ring W is non-aromatic then X₄ is N(R). In some embodiments, if Ring W is non-aromatic then X₄ is O. In some embodiments, if Ring W is non-aromatic then X₄ is S. In some embodiments, if Ring W is non-aromatic then X₄ is S═O. In some embodiments, if Ring W is non-aromatic then X₄ is SO₂.

In some embodiments, X₅ of formula I and/or I(a)-I(n) is a nitrogen atom. In other embodiments, X₅ is a carbon atom. In some embodiments, if X₅ is nitrogen, then the respective R₇′ is absent.

In some embodiments, X₆ of formula I and/or I(a)-I(n) is a nitrogen atom. In other embodiments, X₆ is a carbon atom. In some embodiments, if X₆ is nitrogen, then the respective R₇″ is absent.

In some embodiments, X₇ of formula I and/or I(a)-I(k) and/or I(n) is a nitrogen atom. In other embodiments, X₇ is a carbon atom. In some embodiments, if X₇ is nitrogen, then the respective R₇ is absent.

In some embodiments, X₅ of formula I and/or I(a)-I(n) is a nitrogen atom. In other embodiments, X₈ is a carbon atom. In some embodiments, if X₈ is nitrogen, then the respective R₇′″ is absent.

In some embodiments, X₉ of formula I and/or I(a)-I(n) is a nitrogen atom. In other embodiments, X₉ is a carbon atom. In some embodiments, if X₉ is nitrogen, then the respective R₇″″ is absent.

In some embodiments, X₁₀ of formula I and/or I(a)-I(n) is a nitrogen atom. In other embodiments, X₁₀ is carbon. In other embodiments, X₁₀ is N. In other embodiments, X₁₀ is CH. In other embodiments, X₁₀ is C(R), wherein R is as defined below. In other embodiments, X₁₀ is C(R), wherein R is an alkyl. In other embodiments, X₁₀ is C(R), wherein R is a methyl, a substituted methyl, CH₂—OH, an ethyl, a substituted ethyl, CH₂—CH₂—OH, NH(R₁₀), NH—CH₂-cyclopropyl, COOH, cycloalkyl such as cyclopropyl, alkoxy such as isopropoxy; each represents a separate embodiment according to this invention. In other embodiments, X₁₀ is C(R), wherein R is a substituted alkyl. In other embodiments, X₁₀ is C(R), wherein R is CH₂—OH. In other embodiments, X₁₀ is C(R), wherein R is CH₂—CH₂—OH. In other embodiments, X₁₀ is C(R), wherein R is a cycloalkyl. In other embodiments, X₁₀ is C(R), wherein R is a cyclopropyl. In other embodiments, X₁₀ is C(R), wherein R is not methyl. In other embodiments, X₁₀ is C(R), wherein R is an alkoxy. In other embodiments, X₁₀ is C(R), wherein R is an isopropoxy. In other embodiments, X₁₀ is C(CH₂—OH). In other embodiments, X₁₀ is C(CH₂—CH₂—OH). In other embodiments, X₁₀ is C(R), wherein R is N(H)R₁₀; and R₁₀ is a substituted alkyl. In other embodiments, X₁₀ is C(NH—CH₂-cyclopropyl). In other embodiments, X₁₀ is C(COOH). In other embodiments, X₁₀ is C(CH₃). In other embodiments, X₁₀ is C(cyclopropyl). In other embodiments, X₁₀ is C(isopropoxy). In other embodiments, X₁₀ is C═O. In other embodiments, if X₁₀ is C═O then X₁₁ is N.

In some embodiments, X₁₁ of formula I and/or I(a)-I(n) is a nitrogen atom. In other embodiments, X₁₁ is carbon atom. In other embodiments, X₁₁ is N. In other embodiments, X₁₁ is C. In other embodiments, if X₁₁ is N then X₁₀ is C═O. In other embodiments, if X₁₀ is C═O then X₁₁ is N. In some embodiments, X₁₁ of formula I(j) is CH. In some embodiments, X₁₁ of formula I(j) is C(R).

In some embodiments, X₁₂ of formula I and/or I(a)-I(j) is S. In other embodiments, X₁₂ is not S. In other embodiments, X₁₂ is SO₂. In other embodiments, X₁₂ is O. In other embodiments, X₁₂ is NH. In other embodiments, X₁₂ is N(R). In other embodiments, X₁₂ is N—CH₂—COOH. In other embodiments, X₁₂ is N—CH₂—CH₂—OH. In other embodiments, X₁₂ is N—CH₃. In other embodiments, X₁₂ is N—OH. In other embodiments, X₁₂ is CH═CH. In other embodiments, X₁₂ is CH═CH(R). In other embodiments, X₁₂ is C(R)═CH. In other embodiments, X₁₂ is N═CH. In other embodiments, X₁₂ is N═C(R). In other embodiments, X₁₂ is CH═N. In other embodiments, X₁₂ is C(R)═N. In other embodiments, X₁₂ is N—CH₂CH₃. In other embodiments, X₁₂ is N-iPr. In other embodiments, X₁₂ is N-cyclopropyl. In other embodiments, X₁₂ is N—CH₂-cyclopropyl.

In some embodiments, Ring W′ of formula I(k)-I(m) is aromatic or non-aromatic. In some embodiments, if Ring W′ is aromatic, then X₁₂ is S. In other embodiments, if Ring W′ is aromatic, then X₁₂ is not S. In other embodiments, if Ring W′ is aromatic, then X₁₂ is SO₂. In other embodiments, if Ring W′ is aromatic, then X₁₂ is O. In other embodiments, if Ring W′ is aromatic, then X₁₂ is NH. In other embodiments, if Ring W′ is aromatic, then X₁₂ is N(R). In other embodiments, if Ring W′ is aromatic, then X₁₂ is N—CH₂—COOH. In other embodiments, if Ring W′ is aromatic, then X₁₂ is N—CH₂—CH₂—OH. In other embodiments, if Ring W′ is aromatic, then X₁₂ is N—CH₃. In other embodiments, if Ring W′ is aromatic, then X₁₂ is N—OH. In other embodiments, if Ring W′ is aromatic, then X₁₂ is CH═CH. In other embodiments, if Ring W′ is aromatic, then X₁₂ is CH═CH(R). In other embodiments, if Ring W′ is aromatic, then X₁₂ is C(R)═CH. In other embodiments, if Ring W′ is aromatic, then X₁₂ is N═CH. In other embodiments, if Ring W′ is aromatic, then X₁₂ is N═C(R). In other embodiments, if Ring W′ is aromatic, then X₁₂ is CH═N. In other embodiments, if Ring W′ is aromatic, then X₁₂ is C(R)═N. In other embodiments, if Ring W′ is aromatic, then X₁₂ is N—CH₂CH₃. In other embodiments, if Ring W′ is aromatic, then X₁₂ is N-iPr. In other embodiments, if Ring W′ is aromatic, then X₁₂ is N-cyclopropyl. In other embodiments, if Ring W′ is aromatic, then X₁₂ is N—CH₂-cyclopropyl. In other embodiments, if Ring W′ is non-aromatic, then X₁₂ is CH═CH. In other embodiments, if Ring W′ is non-aromatic, then X₁₂ is CH═CH(R). In other embodiments, if Ring W′ is non-aromatic, then X₁₂ is C(R)═CH. In other embodiments, if Ring W′ is non-aromatic, then X₁₂ is OCH₂. In other embodiments, if Ring W′ is non-aromatic, then X₁₂ is CH₂O. In other embodiments, if Ring W′ is non-aromatic, then X₁₂ is SCH₂. In other embodiments, if Ring W′ is non-aromatic, then X₁₂ is CH₂S. In other embodiments, if Ring W′ is non-aromatic, then X₁₂ is CH═N. In other embodiments, if Ring W′ is non-aromatic, then X₁₂ is C(R)═N. In other embodiments, if Ring W′ is non-aromatic, then X₁₂ is N═CH. In other embodiments, if Ring W′ is non-aromatic, then X₁₂ is N═C(R).

In some embodiments, X₁₃ of formula I(b), I(c), I(f), I(h) and/or I(m) is CH₂. In other embodiments, X₁₃ is CH(R). In other embodiments, X₁₃ is CH—CH₃. In other embodiments, X₁₃ is C(R)₂. In other embodiments, X₁₃ is C═O.

In some embodiments, X₁₄ of formula I(g), I(h), I(l) and/or I(m) is S. In other embodiments, X₁₄ is O. In other embodiments, X₁₄ is N. In other embodiments, X₁₄ is CH. In other embodiments, if X₁₄ is CH then Ring F is not absent. In other embodiments, if X₁₄ is S then R₃ is absent. In other embodiments, if X₁₄ is O then R₃ is absent.

In some embodiments, at least one of X₂, X₃, X₄, X₅, X₆, X₇, X₈ and X₉ of formula I, I(a)-I(c), and/or I(i)-I(k) is a nitrogen atom. In some embodiments, at least one of X₂, X₃, X₄, X₅, X₆, X₈ and X₉ of formula I, and/or I(a)-I(m) is a nitrogen atom. In some embodiments, at least one of X₂, X₃, X₄, X₅, X₆, X₇, X₈ and X₉ of formula I(d) is a nitrogen atom. In some embodiments, at least one of X₂, X₃, X₄, X₅, X₆, X₇, X₈, X₉ and X₁₀ of formula I(d) is a nitrogen atom. In some embodiments, if either one of X₅, X₆, X₇, X₈ and X₉ is nitrogen, then the respective R₇′, R₇″, R₇, R₇′″, and R₇″″ substitution is absent.

In some embodiments, if Ring W is aromatic, then X₁₅ of formula I(k)-I(m) is C. In some embodiments, if Ring W is non-aromatic, then X₁₅ is CH, C(R) or N; each represents a separate embodiment according to this invention. In some embodiments, if Ring W is non-aromatic, then X₁₅ is CH. In some embodiments, if Ring W is non-aromatic, then X₁₅ is C(R). In some embodiments, if Ring W is non-aromatic, then X₁₅ is N.

In some embodiments, R₅ of formula I, I(a), I(d), I(e), I(g) and/or I(i)-I(n) is H. In other embodiments, R₅ is C₁-C₅ linear or branched alkyl. In other embodiments, R₅ is methyl. In other embodiments, R₅ is methyl, ethyl, propyl, isopropyl, butyl, t-butyl, iso-butyl, pentyl, neopentyl; each represents a separate embodiment according to this invention.

In some embodiments, R₅ and R₆ of formula I, I(a), I(d), I(e), I(g), and/or I(i)-I(n) are joined to form a substituted or unsubstituted 5-8 membered heterocyclic ring. In some embodiments, R₅ and R₆ are joined to form a substituted 5-8 membered heterocyclic ring. In some embodiments, R₅ and R₆ are joined to form an unsubstituted 5-8 membered heterocyclic ring. In some embodiments, the heterocyclic ring is azepane, piperazine or 2-(piperazin-1-yl)acetamide; each represents a separate embodiment according to this invention. In some embodiments, the heterocyclic ring is substituted with at least one substitution selected from: F, Cl, Br, I, CF₃, R₂₀, C₁-C₅ linear or branched alkyl, C₁-C₅ linear or branched haloalkyl, OH, alkoxy, R₈—OH (e.g., CH₂—OH), OMe, amide, C(O)N(R)₂, C(O)N(R₁₀)(R₁₁), R₈—C(O)N(R₁₀)(R₁₁), C(O)-pyrrolidine, C(O)-piperidine, N(R)₂, NH(R₁₀), N(R₁₀)(R₁₁), N(CH₃)₂, NH₂, CF₃, aryl, phenyl, heteroaryl, substituted or unsubstituted C₃-C₈ cycloalkyl, cyclobutanol, substituted or unsubstituted 3-8 membered heterocyclic ring, which may be saturated, unsaturated, aromatice, single fused or spiral, pyran, oxetane, piperidine, pyrazole, methyl-pyrrazole, triazole, imidazole, halophenyl, (benzyloxy)phenyl, CN, and NO₂; each is a separate embodiment according to this invention.

In some embodiments, R₆ of formula I, I(a), I(d), I(e), I(g), I(i), I(j)-I(l) and/or I(n) is H. In other embodiments, R₆ is H, F, Cl, Br, I, OH, SH, R₈—OH, R₈—SH, —R₈—O—R₁₀, CH₂—O—CH₃, (CH₂)₂—O—CH₃ (CH₂)₃O—CH₃, (CH₂)₂O—CH(CH₃)₂, R₈—S—R₁₀, (CH₂)₃—S—(CH₂)₂CH₃, R₈—NHC(O)—R₁₀, —O—R₈-R₁₀, R₈-(substituted or unsubstituted C₃-C₈ cycloalkyl), CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted, saturated, unsaturated or aromatic, single, fused or spiro 3-10 membered heterocyclic ring), (CH₂)₃-piperidine, (CH₂)₃-4-fluoro-piperidine, (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-oxa-azaspirodecane, (CH₂)₃-dimethylpyrazole, CH₂-2-oxo-methylpyrrolidine, CH₂-methyl-azetidine, CH₂-azaspiroheptane, CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), (CH₂)₂—NH₂, (CH₂)₃—N(CH₂CH₃)₂, (CH₂)₃—N(CH(CH₃)₂)₂, (CH₂)₃-piperidine, (CH₂)₃-4-fluoro-piperidine, (CH₂)₃-piperidine-2-one, (CH₂)₃-4-cyano-piperidine, (CH₂)₃-4-trifluoromethyl-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃), R₈—C(O)N(R₁₀)(R₁₁), (CH₂)₂—C(O)-piperidine, R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)CH₃), C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂, C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy methoxy, optionally wherein at least one methylene group (CH₂) in the alkoxy is replaced with an oxygen atom, O—(CH₂)₂O—CH₃, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, cyclopropyl, cyclobutyl, cyclohexyl, methoxycyclopropyl, methylcyclobutyl, cyclopropyl, aminomethyl-cyclobutyl, methoxycyclobutyl, 2,3-dihydro-1H-indenol, R₈-(substituted or unsubstituted C₃-C₈ cycloalkyl), substituted or unsubstituted 3-8 membered heterocyclic ring (e.g., piperidine, 1-methyl-piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), or substituted or unsubstituted benzyl; each represents a separate embodiment according to this invention. In some embodiments, R₆ may be further substituted with at least one substitution selected from: F, Cl, Br, I, C₁-C₅ linear or branched alkyl, OH, alkoxy, OMe, amide, C(O)N(R)₂, C(O)-alkyl, C(O)-pyrrolidine, C(O)-piperidine, N(R)₂, NH(R₁₀), N(R₁₀)(R₁₁), N(CH₃)₂, NH₂, CF₃, aryl, phenyl, heteroaryl, substituted or unsubstituted C₃-C₈ cycloalkyl, cyclobutanol, substituted or unsubstituted 3-8 membered heterocyclic ring pyran, oxetane, piperidine, pyrazole, methyl-pyrrazole, triazole, imidazole, halophenyl, (benzyloxy)phenyl, CN, and NO₂; each represents a separate embodiment according to this invention. In some embodiments, R₆ is H. In some embodiments, R₆ is —R₈—O—R₁₀. In some embodiments, R₆ is CH₂—O—CH₃. In some embodiments, R₆ is R₈—S—R₁₀. In some embodiments, R₆ is (CH₂)₃—S—(CH₂)₂CH₃. In some embodiments, R₆ is R₈—NHC(O)—R₁₀. In some embodiments, R₆ is (CH₂)₃—NHC(O)—R₁₀. In some embodiments, R₆ is (CH₂)—NHC(O)—R₁₀. In some embodiments, R₆ is R₈-(substituted or unsubstituted C₃-C₈ cycloalkyl). Examples of R₈-(substituted or unsubstituted C₃-C₈ cycloalkyl) include but not limited to: CH₂-cyclobutanol, CH₂-difluorocyclopropyl, CH₂-methylcyclopropyl, CH₂-dimethylamino-cyclohexyl, (CH₂)₂-cyclopentanole, and CH₂-cyclohexanol; each represents a separate embodiment according to this invention. In some embodiments, R₆ is R₈-(substituted or unsubstituted saturated, unsaturated or aromatic, single, fused or spiro 3-8 membered heterocyclic ring). In some embodiments, R₆ is R₈-(substituted or unsubstituted saturated, single 3-8 membered heterocyclic ring). In some embodiments, R₆ is R₈-(substituted or unsubstituted unsaturated, single 3-8 membered heterocyclic ring). In some embodiments, R₆ is R₈-(substituted or unsubstituted aromatic, single 3-8 membered heterocyclic ring). In some embodiments, R₆ is R₈-(substituted or unsubstituted saturated, fused 3-8 membered heterocyclic ring). In some embodiments, R₆ is R₈-(substituted or unsubstituted unsaturated, fused 3-8 membered heterocyclic ring). In some embodiments, R₆ is R₈-(substituted or unsubstituted aromatic, fused 3-8 membered heterocyclic ring). In some embodiments, R₆ is R₈-(substituted or unsubstituted spiro 3-8 membered heterocyclic ring). Examples of R₈-(substituted or unsubstituted saturated, unsaturated or aromatic, single, fused or spiro 3-8 membered heterocyclic ring) include but not limited to: (CH₂)₃-piperidine, (CH₂)₃-4-fluoro-piperidine, (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-oxa-azaspirodecane, (CH₂)₃-dimethylpyrazole, CH₂-2-oxo-methylpyrrolidine, CH₂-methyl-azetidine, and CH₂-azaspiroheptane. In some embodiments, R₆ is NH₂. In some embodiments, R₆ is NHR. In some embodiments, R₆ is N(R)₂. In some embodiments, R₆ is NH(R₁₀). In some embodiments, R₆ is N(R₁₀)(R₁₁). In some embodiments, R₆ is R₈—N(R₁₀)(R₁₁). In some embodiments, R₈—N(R₁₀)(R₁₁) includes but not limited to: (CH₂)₃—N(CH₂CH₃)₂, (CH₂)₃—N(CH(CH₃)₂)₂, (CH₂)₃-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₂—NH₂, (CH₂)₃—NH₂, and (CH₂)₃—N(CH₂CH₃)(CH₂CF₃). In some embodiments, R₆ is R₈—C(O)N(R₁₀)(R₁₁) such as (CH₂)₂—C(O)-piperidine. In some embodiments, R₆ is C₁-C₅ linear or branched, substituted or unsubstituted alkyl. Examples of C₁-C₅ linear or branched, substituted or unsubstituted alkyl include but not limited to: CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), CH(CH₃)C(O)N(CH₃)₂, benzyl, methyl, ethyl, and CH₂—OCH₂—CH₂—O—CH₃. In some embodiments, R₆ is methyl. In some embodiments, R₆ is substituted or unsubstituted C₃-C₈ cycloalkyl. In some embodiments, substituted or unsubstituted C₃-C₈ cycloalkyl include: cyclopropyl, cyclobutyl, cyclohexyl, methoxycyclopropyl, methylcyclobutyl, cyclopropyl, aminomethyl-cyclobutyl, methoxycyclobutyl and 2,3-dihydro-1H-indeno. In some embodiments, R₆ is R₈-(substituted or unsubstituted C₃-C₈ cycloalkyl). In some embodiments, R₆ is substituted or unsubstituted saturated, unsaturated or aromatic, single, fused or spiro 3-10 membered heterocyclic ring. In some embodiments, the substituted or unsubstituted saturated, unsaturated or aromatic, single, fused or spiro 3-10 membered heterocyclic ring is piperidine, 1-methyl-piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane; each represents a separate embodiment according to this invention. In some embodiments, R₆ is piperidine. In some embodiments, R₆ is 1-methyl-piperidine. In some embodiments, R₆ is tetrahydropyran. In some embodiments, R₆ is substituted or unsubstituted R₈-aryl, such as benzyl. In some embodiments, R₆ may be further substituted by at least one substitution selected from: F, Cl, Br, I, CF₃, R₂₀, C₁-C₅ linear or branched alkyl, C₁-C₅ linear or branched haloalkyl, OH, alkoxy, R₈—OH (e.g., CH₂—OH), OMe, amide, C(O)N(R)₂, C(O)N(R₁₀)(R₁₁), R₈—C(O)N(R₁₀)(R₁₁), C(O)-pyrrolidine, C(O)-piperidine, N(R)₂, NH(R₁₀), N(R₁₀)(R₁₁), N(CH₃)₂, NH₂, CF₃, aryl, phenyl, heteroaryl, substituted or unsubstituted C₃-C₈ cycloalkyl, cyclobutanol, substituted or unsubstituted 3-8 membered heterocyclic ring, which may be saturated, unsaturated, aromatice, single fused or spiral, pyran, oxetane, piperidine, pyrazole, methyl-pyrrazole, triazole, imidazole, halophenyl, (benzyloxy)phenyl, CN, and NO₂; each is a separate embodiment according to this invention.

In some embodiments, R₆ and R₅ of formula I, I(a), I(d), I(e), I(g), I(i), I(j)-I(l) and/or I(n) are joined to form a substituted or unsubstituted saturated, unsaturated or aromatic, single, fused or spiro 5-8 membered heterocyclic ring. In some embodiments, the substituted or unsubstituted saturated, unsaturated or aromatic, single, fused or spiro 5-8 membered heterocyclic ring is azepane, piperazine, or 2-(piperazin-1-yl)acetamide; each represents a separate embodiment according to this invention. In some embodiments, the ring may be further substituted by at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

In some embodiments, R₆ of formula I, I(a), I(d), I(e), I(g), I(i), I(j)-I(l) and/or I(n) is represented by the structure of formula B:

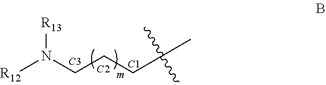

wherein
m is 0 or 1; and
   R₁₂ is R₂₀ or C₁-C₅ C(O)-alkyl, and R₁₃ is R₃₀; or
   R₁₂ and R₁₃ are both H; or
   R₁₂ and R₁₃ are each independently H or substituted or unsubstituted C₁-C₅ alkyl (e.g., ethyl, trifluoroethyl); or
   R₁₂ and C3 are joined to form ring A and R₁₃ is R₃₀; or
   R₁₂ and R₁₃ are joined to form ring B; or
   R₁₂ and C1 are joined to form ring C and R₁₃ is R₃₀; or
   C1 and C3 are joined to form ring D and R₁₂ and R₁₃ are each independently R₃₀; or
   R₁₃ and C2 are joined to form ring E, m is 1, and R₁₂ is R₃₀; or
   R₁₂ and R₁₃ are joined to form ring B and C1 and C3 are joined to form ring D;
   wherein
      Ring A, C and E are each independently a substituted or unsubstituted single spiro or fused 3-8 membered heterocyclic rings;
      Ring B is a substituted or unsubstituted single, spiro or fused 3-8 membered heterocyclic ring; and
      Ring D is a substituted or unsubstituted C₃-C₅ cycloalkyl;

In some embodiments, formula B is represented by formula Bi.

In some embodiments, R₆ of formula I, I(a), I(d), I(e), I(g), I(i), I(j)-I(l) and/or I(n) is represented by the structure of formula Bi:

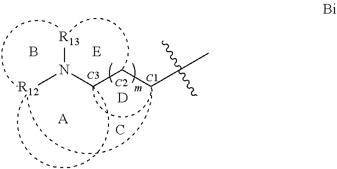

wherein
m is 0 or 1; and
   R₁₂ is R₂₀ or C₁-C₅ C(O)-alkyl, and R₁₃ is R₃₀; or
   R₁₂ and R₁₃ are both H; or
   R₁₂ and R₁₃ are each independently H or substituted or unsubstituted C₁-C₈alkyl (e.g., ethyl, trifluoroethyl); or
   R₁₂ and C3 are joined to form ring A and R₁₃ is R₃₀; or
   R₁₂ and R₁₃ are joined to form ring B; or
   R₁₂ and C1 are joined to form ring C and R₁₃ is R₃₀; or
   C1 and C3 are joined to form ring D and R₁₂ and R₁₃ are each independently R₃₀; or
   R₁₃ and C2 are joined to form ring E, m is 1, and R₁₂ is R₃₀; or
   R₁₂ and R₁₃ are joined to form ring B and C1 and C3 are joined to form ring D;
   wherein
      Ring A, C and E are each independently a substituted or unsubstituted single spiro or fused 3-8 membered heterocyclic rings;
      Ring B is a substituted or unsubstituted single, spiro or fused 3-8 membered heterocyclic ring; and
      Ring D is a substituted or unsubstituted C₃-C₈ cycloalkyl;

In some embodiments, R₁₂ of formula B and/or Bi is H. In some embodiments, R₁₂ is R₂₀. In other embodiments, R₁₂ is R₃₀. In some embodiments, R₁₂ is C₁-C₅ C(O)-alkyl. In some embodiments, R₁₂ is substituted or unsubstituted C₁-C₅ alkyl. In some embodiments, R₁₂ is unsubstituted C₁-C₅ alkyl. In some embodiments, the alkyl is ethyl. In some embodiments, R₁₂ is substituted C₁-C₅ alkyl. In some embodiments, the alkyl is trifluoroethyl.

In some embodiments, R₁₃ of formula B and/or Bi is H. In other embodiments, R₁₃ is R₃₀. In some embodiments, R₁₃ is substituted or unsubstituted C₁-C₅ alkyl. In some embodiments, R₁₃ is unsubstituted C₁-C₅ alkyl. In some embodiments, the alkyl is ethyl. In some embodiments, R₁₃ is substituted C₁-C₅ alkyl. In some embodiments, the alkyl is trifluoroethyl.

In some embodiments, R₆ of formula I, I(a), I(d), I(e), I(g), I(i), I(j)-I(l) and/or I(n) is represented by formula B. In some embodiments, R₁₂ of formula B is R₂₀ or C₁-C₅ C(O)-alkyl, and R₁₃ is R₃₀. In some embodiments, R₁₂ and R₁₃ of formula B are both H. In some embodiments, R₁₂ and R₁₃ of formula B are each independently H or substituted or unsubstituted C₁-C₅ alkyl (e.g., ethyl, trifluoroethyl). In some embodiments, R₁₂ and R₁₃ of formula B are each independently H or trifluoroethyl. In some embodiments, R₁₂ and C3 of formula B are joined to form ring A and R₁₃ is R₁₀. In some embodiments, R₁₂ and R₁₃ of formula B are joined to form ring B. In some embodiments, R₁₂ and C1 of formula B are joined to form ring C and R₁₃ is R₃₀. In some embodiments, C1 and C3 of formula B are joined to form ring D and R₁₂ and R₁₃ of formula B are each independently R₃₀. In some embodiments, R₁₃ and C2 of formula B are joined to form ring E, m is 1, and R₁₂ of formula B is R₃₀. In some embodiments, R₁₂ and R₁₃ of formula B are joined to form ring B and C1 and C3 of formula B are joined to form ring D.

In some embodiments, R₆ of formula I, I(a), I(d), I(e), I(g), I(i), I(j)-I(l) and/or I(n) is represented by formula Bi. in some embodiments, R₁₂ of formula Bi is R₂₀ or C₁-C₅ C(O)-alkyl, and R₁₃ is R₃₀. In some embodiments, R₁₂ and R₁₃ of formula Bi are both H. In some embodiments, R₁₂ and R₁₃ of formula Bi are each independently H or substituted or unsubstituted C₁-C₅ alkyl (e.g., ethyl, trifluoroethyl). In some embodiments, R₁₂ and R₁₃ of formula Bi are each independently H or trifluoroethyl. In some embodiments, R₁₂ and C3 of formula Bi are joined to form ring A and R₁₃ is R₃₀. In some embodiments, R₁₂ and R₁₃ of formula Bi are joined to form ring B. In some embodiments, R₁₂ and C1 of formula Bi are joined to form ring C and R₁₃ is R₃₀. In some embodiments, C1 and C3 of formula Bi are joined to form ring D and R₁₂ and R₁₃ of formula Bi are each independently R₃₀. In some embodiments, R₁₃ and C2 of formula Bi are joined to form ring E, m is 1, and R₁₂ of formula Bi is R₃₀. In some embodiments, R₁₂ and R₁₃ of formula Bi are joined to form ring B and C1 and C3 of formula Bi are joined to form ring D.

In some embodiments, R₆ of formula I, I(a), I(d), I(e), I(g), I(i), I(j)-I(l) and/or I(n) is represented by formula Bi and/or B and
R₁₂ of formula Bi and/or B is R₂₀ or C₁-C₅ C(O)-alkyl, and R₁₃ of formula Bi and/or B is R₃₀; or
R₁₂ and R₁₃ are both H, or
R₁₂ and R₁₃ are each independently H or trifluoroethyl; or
R₁₂ and C3 are joined to form ring A and R₁₃ is R₃₀; or
R₁₂ and R₁₃ are joined to form a substituted or unsubstituted pyrrolidine ring, piperazine, thiomorpholine 1,1-dioxide 2-oxa-6-azaspiro[3.3]heptane, pyrazole, imidazole, 2,5-diazabicyclo[2.2.1]heptane or a diazabicyclo[2.2.1]heptane; or
R₁₂ and C1 are joined to form ring C and R₁₃ is R₃₀; or
C1 and C3 are joined to form ring D and R₁₂ and R₁₃ are each independently R₃₀; or
R₁₃ and C2 are joined to form ring E, m is 1, and R₁₂ is R₃₀; or
R₁₂ and R₁₃ are joined to form ring B and C1 and C3 are joined to form ring D.

In some embodiments, R₆ of formula I, I(a), I(d), I(e), I(g), I(i), I(j)-I(l) and/or I(n) is represented by formula Bi and/or B and
R₁₂ of formula Bi and/or B is R₂₀ or C₁-C₅ C(O)-alkyl, and R₁₃ of formula Bi and/or B is R₃₀; or
R₁₂ and C3 are joined to form ring A and R₁₃ is R₃₀; or
R₁₂ and R₁₃ are joined to form a substituted or unsubstituted pyrrolidine ring, piperazine, thiomorpholine 1,1-dioxide 2-oxa-6-azaspiro[3.3]heptane, pyrazole, imidazole, 2,5-diazabicyclo[2.2.1]heptane or a diazabicyclo[2.2.1]heptane; or
R₁₂ and C1 are joined to form ring C and R₁₃ is R₃₀; or
C1 and C3 are joined to form ring D and R₁₂ and R₁₃ are each independently R₃₀; or
R₁₃ and C2 are joined to form ring E, m is 1, and R₁₂ is R₃₀; or
R₁₂ and R₁₃ are joined to form ring B and C1 and C3 are joined to form ring D.

In some embodiments, ring A of formula Bi, is a substituted or unsubstituted single spiro or fused 3-8 membered heterocyclic ring. In some embodiments, ring A, is an unsubstituted single 3-8 membered heterocyclic ring. In some embodiments, ring A, is an unsubstituted spiro 3-8 membered heterocyclic ring. In some embodiments, ring A, is an unsubstituted fused 3-8 membered heterocyclic ring. In some embodiments, ring A, is a substituted single 3-8 membered heterocyclic ring. In some embodiments, ring A, is a substituted spiro 3-8 membered heterocyclic ring. In some embodiments, ring A, is a substituted fused 3-8 membered heterocyclic ring. In some embodiments, ring A is: pyrrolidine, methylpyrrolidine, ethylpyrrolidine, 2-oxopyrrolidine, piperidine, methylpiperidine, methyl-2-oxopyrrolidine, pyran-azetidine, methyl-azetidine, azabicyclooctane, 2-azabicyclo[2.1.1]hexane, or 2-azaspiro[3.3]heptane; each represents a separate embodiment according to this invention. In some embodiments, ring A is: pyrrolidine, methylpyrrolidine, or ethylpyrrolidine; each represents a separate embodiment according to this invention.

In some embodiments, ring B of formula Bi, is a substituted or unsubstituted single spiro or fused 3-8 membered heterocyclic ring. In some embodiments, ring B, is an unsubstituted single 3-8 membered heterocyclic ring. In some embodiments, ring B, is an unsubstituted spiro 3-8 membered heterocyclic ring. In some embodiments, ring B, is an unsubstituted fused 3-8 membered heterocyclic ring. In some embodiments, ring B, is a substituted single 3-8 membered heterocyclic ring. In some embodiments, ring B, is a substituted spiro 3-8 membered heterocyclic ring. In some embodiments, ring B, is a substituted fused 3-8 membered heterocyclic ring. In some embodiments, ring B is: pyrrolidine, methylpyrrolidine, ethylpyrrolidine, 2-oxopyrrolidine, hydroxymethyl-pyrrolidine, piperidine, piperidin-2-one, 4-fluoropiperidin-2-one, piperidine-4-carbonitrile, methylpiperidine, fluoropiperidine, 4-fluoropiperidine, 4-fluoro-2-methylpiperidine, difluoropiperidine, piperazine, methyl-piperazine, dimethyl-pyrazole, methyl-2-oxopyrrolidine, pyran-, azetidine, methyl-azetidine, imidazole, azabicyclooctane, 2-azabicyclo[2.1.1]hexane, or 2-azaspiro[3.3]heptane, diazabicyclo[2.2.1]heptane, 2-methyl-2,5-diazabicyclo[2.2.1]heptane, thiomorpholine, or 1,1-dioxide-2-oxa-6-azaspiro[3.3]heptane; each represents a separate embodiment according to this invention. In some embodiments, ring B is: piperidine, methyl-piperidin, fluoropiperidine, difluoropiperidine, pyrrolidine, piperazine, methylpyrrolidine, thiomorpholine, methyl-piperazine, dimethyl-pyrazole, imidazole, 2-methyl-2,5-diazabicyclo[2.2.1]heptane, 1,1-dioxide-2-oxa-6-azaspiro[3.3]heptane, hydroxymethyl-pyrrolidine or diazabicyclo[2.2.1]heptane, 6-fluoro-3-azabicyclo[3.1.1]heptane; each represents a separate embodiment according to this invention. In some embodiments, ring B is 4-fluoropiperidine.

In some embodiments, ring C of formula Bi, is a substituted or unsubstituted single spiro or fused 3-8 membered heterocyclic ring. In some embodiments, ring C, is an unsubstituted single 3-8 membered heterocyclic ring. In some embodiments, ring C, is an unsubstituted spiro 3-8 membered heterocyclic ring. In some embodiments, ring C, is an unsubstituted fused 3-8 membered heterocyclic ring. In some embodiments, ring C, is a substituted single 3-8 membered heterocyclic ring. In some embodiments, ring C, is a substituted spiro 3-8 membered heterocyclic ring. In some embodiments, ring C, is a substituted fused 3-8 membered heterocyclic ring. In some embodiments, ring C is: pyrrolidine, methylpyrrolidine, ethylpyrrolidine, 2-oxopyrrolidine, piperidine, methylpiperidine, methyl-2-oxopyrrolidine, pyran-azetidine, methyl-azetidine, azabicyclooctane, 2-azabicyclo[2.1.1]hexane, or 2-azaspiro[3.3]heptane; each represents a separate embodiment according to this invention. In some embodiments, ring C is: piperidine, pyrrolidine, methyl-2-oxopyrrolidine, pyran-pyrrolidine, methyl-azetidine, azabicyclooctane, 2-azabicyclo[2.1.1]hexane, or 2-azaspiro[3.3]heptane; each represents a separate embodiment according to this invention.

In some embodiments, ring D of formula Bi, is a substituted or unsubstituted C₃-C₅ cycloalkyl.

In some embodiments, ring D, is a substituted C₃-C₅ cycloalkyl. In some embodiments, ring D, is an unsubstituted C₃-C₅ cycloalkyl. In some embodiments, ring D is cyclopropane, cyclobutane, cyclopentane, cyclohexane or cycloheptane; each represents a separate embodiment according to this invention.

In some embodiments, ring E of formula Bi, is a substituted or unsubstituted single spiro or fused 3-8 membered heterocyclic ring. In some embodiments, ring E, is an unsubstituted single 3-8 membered heterocyclic ring. In some embodiments, ring E, is an unsubstituted spiro 3-8 membered heterocyclic ring. In some embodiments, ring E, is an unsubstituted fused 3-8 membered heterocyclic ring. In some embodiments, ring E, is a substituted single 3-8 membered heterocyclic ring. In some embodiments, ring E, is a substituted spiro 3-8 membered heterocyclic ring. In some embodiments, ring E, is a substituted fused 3-8 membered heterocyclic ring. In some embodiments, ring E is: pyrrolidine, methylpyrrolidine, ethylpyrrolidine, 2-oxopyrrolidine, piperidine, methylpiperidine, methyl-2-oxopyrrolidine, pyran-azetidine, methyl-azetidine, azabicyclooctane, 2-azabicyclo[2.1.1]hexane, or 2-azaspiro[3.3]heptane; each represents a separate embodiment according to this invention. In some embodiments, ring E is: pyrrolidine, azetidine, ethylpyrrolidine, oxopyrrolidine, or methylpiperidine; each represents a separate embodiment according to this invention.

In some embodiments, R₆ of formula I, I(a), I(d), I(e), I(g), I(i), I(j)-I(l) and/or I(n) is F, Cl, Br, I, OH, SH, R₈—OH, R₈—SH, —R₈—O—R₁₀ (e.g., CH₂—O—CH₃), R₈—S—R₁₀ (e.g., (CH₂)₃—S—(CH₂)₂CH₃), R₈—NHC(O)—R₁₀, —O—R₈-R₁₀, R₈-(substituted or unsubstituted C₃-C₈ cycloalkyl) (e.g., CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), (CH₂)₃-pyran, CH₂-tetrahydrofurane, CH₂-dioxane, CH₂-methyl-THF, CH₂-oxa-azaspirodecane, (CH₂)₃-dimethylpyrazole, CH₂-methyl-azetidine, CH₂-azaspiroheptane, CF₃, CD₃, OCD₃, CN, NO₂, —CH₂CN, —R₈CN, NH₂, NHR, N(R)₂, NH(R₁₀), N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)CH₃), C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., methoxycyclopropyl, methylcyclobutyl, cyclopropyl, aminomethyl-cyclobutyl, methoxycyclobutyl, 2,3-dihydro-1H-indenol), substituted or unsubstituted 3-8 membered heterocyclic ring (e.g., trifluoromethyl-oxetane, hydroxy-tetrahydrofurane, 1-methylazepan-2-one, 3-azabicyclo[3.1.0]hexane), substituted or unsubstituted aryl, or substituted or unsubstituted benzyl; each represents a separate embodiment according to this invention. In some embodiments, R₆ may be further substituted with at least one substitution selected from: F, Cl, Br, I, C₁-C₅ linear or branched alkyl, OH, alkoxy (e.g., OMe), amide (e.g., C(O)N(R)₂), C(O)-alkyl, C(O)-pyrrolidine, C(O)-piperidine, N(R)₂ (e.g., N(CH₃)₂, NH₂), NH(R₁₀), N(R₁₀)(R₁₁), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN, and NO₂; each represents a separate embodiment according to this invention.

In some embodiments, R₆ of formula I, I(a), I(d), I(e), I(g), I(i), I(j)-I(l) and/or I(n) is —R₈—O—R₁₀. In some embodiments, —R₈—O—R₁₀ is CH₂—O—CH₃. In some embodiments, R₆ is R₈—S—R₁₀. In some embodiments, R₈—S—R₁₀ is (CH₂)₃—S—(CH₂)₂CH₃. In some embodiments, R₆ is R₈—NHC(O)—R₁₀. In some embodiments, R₆ is R₈-(substituted or unsubstituted C₃-C₈ cycloalkyl). In some embodiments, the R₈-(substituted or unsubstituted C₃-C₈ cycloalkyl) is CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol). each represents a separate embodiment according to this invention. In some embodiments, R₆ is R₈-(substituted or unsubstituted, saturated, unsaturated or aromatic, single, fused or spiro 3-10 membered heterocyclic ring). In some embodiments, R₆ is (CH₂)₃-piperidine. In some embodiments, R₆ is (CH₂)₂—NH₂. In some embodiments, R₆ is (CH₂)₃—NH₂. In some embodiments, R₆ is (CH₂)₃-4-fluoro-piperidine. In some embodiments, R₆ is (CH₂)₃-pyran, CH₂-tetrahydrofurane, CH₂-dioxane, CH₂-methyl-THF, CH₂-oxa-azaspirodecane, (CH₂)₃-dimethylpyrazole, CH₂-methyl-azetidine, or CH₂-azaspiroheptane; each represents a separate embodiment according to this invention. In some embodiments, R₆ is C₁-C₅ linear or branched, substituted or unsubstituted alkyl. In some embodiments, R₆ is C₁-C₅ linear or branched, substituted alkyl. In some embodiments, the substituted alkyl is CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), CH₂—OCH₂—CH₂—O—CH₃ or benzyl; each represents a separate embodiment according to this invention. In some embodiments, R₆ is C₁-C₅ linear or branched, unsubstituted alkyl. In some embodiments, the unsubstituted alkyl is methyl, ethyl, propyl, isopropyl, butyl, isobutyl, tert-butyl, pentyl, or neopentyl; each represents a separate embodiment according to this invention. In some embodiments, R₆ is substituted or unsubstituted C₃-C₈ cycloalkyl. In some embodiments, R₆ is substituted C₃-C₈ cycloalkyl. In some embodiments, the substituted cycloalkyl is methoxycyclopropyl, methylcyclobutyl, aminomethyl-cyclobutyl, or methoxycyclobutyl, 2,3-dihydro-1H-indenol; each represents a separate embodiment according to this invention. In some embodiments, R₆ is unsubstituted C₃-C₈ cycloalkyl. In some embodiments, the unsubstituted cycloalkyl is cyclopropyl, cyclobutyl, cyclopentyl, cyclohexyl, cycloheptyl or cyclooctyl; each represents a separate embodiment according to this invention. In some embodiments, R₆ is substituted or unsubstituted 3-8 membered heterocyclic ring. In some embodiments, the substituted heterocyclic ring is piperidine, 1-methyl-piperidine, tetrahydropyran, trifluoromethyl-oxetane, hydroxy-tetrahydrofurane, 1-methylazepan-2-one, or 3-azabicyclo[3.1.0]hexane; each represents a separate embodiment according to this invention. In some embodiments, R₆ is piperidine. In some embodiments, R₆ is 1-methyl-piperidine. In some embodiments, R₆ is tetrahydropyran.

In some embodiments, R₇ of formula I, I(a)-I(c), I(i), I(j), I(k) and/or I(n) is H, F, Cl, Br, I, OH, O—R₂₀, SH, R₈—OH, R₈—SH, SR₁₀, —R₈—O—R₁₀, —R₈—S—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈-N(R₁₀)(R₁₁), R₉—R₈-N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), C(O)NH(CH₃), SO₂R, SO₂N(R₁₀)(R₁₁), CH(CF₃)(NH—R₁₀), C₁-C₅ linear or branched, substituted or unsubstituted alkyl, methyl, C₁-C₅ linear or branched, substituted or unsubstituted alkenyl, C₁-C₅ linear or branched, or C₃-C₅ cyclic haloalkyl, C₁-C₅ linear or branched, or C₃-C₅ cyclic alkoxy optionally wherein at least one methylene group (CH₂) in the alkoxy is replaced with an oxygen atom, C₁-C₅ linear or branched thioalkyl, C₁-C₅ linear or branched thioalkoxy, C₁-C₅ linear or branched haloalkoxy, C₁-C₅ linear or branched alkoxyalkyl, substituted or unsubstituted C₃-C₅ cycloalkyl, substituted or unsubstituted 3-8 membered heterocyclic ring, substituted or unsubstituted aryl, or substituted or unsubstituted benzyl; each represents a separate embodiment according to this invention. In some embodiments, R₇ is further substituted with at least one substitution selected from: F, Cl, Br, I, C₁-C₅ linear or branched alkyl, OH, alkoxy (e.g., OMe), amide (e.g., C(O)N(R)₂), C(O)-alkyl, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₅ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

In some embodiments, R₇ of formula I, I(a)-I(c), I(i), I(j), I(k) and/or I(n) is H. In some embodiments, R₇ is F. In some embodiments, R₇ is Cl. In some embodiments, R₇ is Br. In some embodiments, R₇ is I. In some embodiments, R₇ is OH. In some embodiments, R₇ is O—R₂₀. In some embodiments, R₇ is CF₃. In some embodiments, R₇ is CN. In some embodiments, R₇ is NH₂. In some embodiments, R₇ is NHR. In some embodiments, R₇ is N(R)₂. In some embodiments, R₇ is NH(R₁₀). In some embodiments, R₇ is N(R₁₀)(R₁₁). In some embodiments, R₇ is NHC(O)—R₁₀. In some embodiments, R₇ is COOH. In some embodiments, R₇ is —C(O)Ph. In some embodiments, R₇ is C(O)O—R₁₀. In some embodiments, R₇ is C(O)H. In some embodiments, R₇ is C(O)—R₁₀. In some embodiments, R₇ is C₁-C₅ linear or branched C(O)-haloalkyl. In some embodiments, R₇ is —C(O)NH₂. In some embodiments, R₇ is C(O)NHR. In some embodiments, C(O)NHR is C(O)NH(CH₃). In some embodiments, R₇ is C(O)N(R₁₀)(R₁₁). In some embodiments, C(O)N(R₁₀)(R₁₁) is C(O)NH(CH₃), C(O)NH(CH₂CH₂OCH₃), or C(O)NH(CH₂CH₂OH); each represents a separate embodiment according to this invention. In some embodiments, R₇ is SO₂R. In some embodiments, R₇ is C₁-C₅ linear or branched, substituted or unsubstituted alkyl. In some embodiments, the alkyl is methylimidazole, methyl, ethyl, propyl, isopropyl, butyl, isobutyl, tert-butyl, pentyl, neopentyl or hexyl; each represents a separate embodiment according to this invention. In some embodiments, R₇ is C₁-C₅ linear or branched, or C₃-C₅ cyclic haloalkyl. In some embodiments, R₇ is C₁-C₅ linear haloalkyl. In some embodiments, the haloalkyl is CHF₂. In some embodiments, R₇ is C₁-C₅ branched haloalkyl. In some embodiments, R₇ is C₃-C₅ cyclic haloalkyl. In some embodiments, R₇ is C₁-C₅ linear or branched, or C₃-C₅ cyclic alkoxy optionally wherein at least one methylene group (CH₂) in the alkoxy is replaced with an oxygen atom. In some embodiments, R₇ is C₁-C₅ linear alkoxy. In some embodiments, the alkoxy is methoxy. In some embodiments, the alkoxy is ethoxy. In some embodiments, R₇ is C₁-C₅ branched alkoxy. In some embodiments, R₇ is C₃-C₈ cyclic alkoxy. In some embodiments, R₇ is C₁-C₅ linear or branched thioalkyl. In some embodiments, R₇ is C₁-C₅ linear or branched haloalkoxy. In some embodiments, R₇ is C₁-C₅ linear haloalkoxy. In some embodiments, R₇ is C₁-C₅ branched haloalkoxy. In some embodiments, R₇ is C₁-C₅ linear or branched alkoxyalkyl. In some embodiments, R₇ is substituted or unsubstituted C₃-C₅ cycloalkyl. In some embodiments, the cycloalkyl is cyclopropyl, cyclopropanol, cyclobutyl, cyclopentyl, cyclohexyl, cycloheptyl or cyclooctyl; each represents a separate embodiment according to this invention. In some embodiments, R₇ is substituted or unsubstituted 3-8 membered heterocyclic ring. In some embodiments, R₇ is unsubstituted 3-8 membered heterocyclic ring. In some embodiments, R₇ is substituted 3-8 membered heterocyclic ring. In some embodiments, R₇ is substituted or unsubstituted 4-7 membered heterocyclic ring. In some embodiments, R₇ is unsubstituted 4-7 membered heterocyclic ring. In some embodiments, R₇ is substituted 4-7 membered heterocyclic ring. In some embodiments, the heterocyclic ring is morpholine (e.g., 2 or 3-morpholine), tetrahydrofuran, tetrahydropyran, oxetane, oxetan-3-ol, pyrrolidine, pyrrolidine-3-ol, 1-methylpyrrolidine, pyrrolidin-2-one, pyrrolidinone, imidazole, pyrazole, piperazine, piperidine, piperidine-4-ol, piperidine-4-carbonitrile, 4-fluoropiperidine, oxadiazole, triazole, 2-oxopyrrolidine, pyridine, or 1-methylpyridine; each represents a separate embodiment according to this invention. In some embodiments, R₇ is R₈-(substituted or unsubstituted single, fused or spiro 3-8 membered heterocyclic ring). In some embodiments, R₇ is R₈-(unsubstituted single 3-8 membered heterocyclic ring). In some embodiments, R₇ is R₈-(unsubstituted fused 3-8 membered heterocyclic ring). In some embodiments, R₇ is R₈-(unsubstituted spiro 3-8 membered heterocyclic ring). In some embodiments, R₇ is R₈-(substituted single 3-8 membered heterocyclic ring). In some embodiments, R₇ is R₈-(substituted fused 3-8 membered heterocyclic ring). In some embodiments, R₇ is R₈-(substituted spiro 3-8 membered heterocyclic ring). In some embodiments, the heterocyclic ring may be saturated. In some embodiments, the heterocyclic ring may be unsaturated. In some embodiments, the hetrocyclic ring may be aromatic. In some embodiments, R₇ is substituted or unsubstituted aryl. In some embodiments, R₇ is phenyl. In some embodiments, R₇ may be further substituted with at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₅ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

In some embodiments, R₇ of formula I, I(a)-I(c), I(i), I(j), I(k) and/or I(n) is O—R₂₀. In some embodiments, R₇ is substituted or unsubstituted 4-7 membered heterocyclic ring. In some embodiments, R₇ is unsubstituted 4-7 membered heterocyclic ring. In some embodiments, R₇ is substituted 4-7 membered heterocyclic ring. In some embodiments, the heterocyclic ring is morpholine, (e.g., 2 or 3-morpholine), pyran, oxetane, pyrrolidine, pyrrolidine-3-ol, tetrahydrofuran, imidazole, piperazine, piperidine, piperidine-4-ol, dioxazole, triazole, pyridine, 1-methylpyridine, or 2-oxopyrrolidine; each represents a separate embodiment according to this invention. In some embodiments, R₇ is substituted or unsubstituted aryl. In some embodiments, R₇ is phenyl. In some embodiments, R₇ may be further substituted with at least one substitution selected from F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

In some embodiments, R₇ of formula I, I(a)-I(c), I(i), I(j), I(k) and/or I(n) is not H, F, Cl, C₁-C₅ linear or branched, or C₃-C₈ cyclic alkoxy, C₁-C₅ linear or branched haloalkoxy or C₁-C₅ linear or branched, substituted or unsubstituted alkyl.

In some embodiments, R₇ of formula I, I(a)-I(c), I(i), I(j), I(k) and/or I(n) is represented by the structure of formula A:

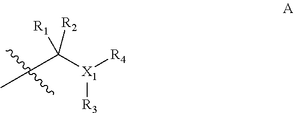

wherein
   X₁ is N or O;
   R₁ and R₂ are each independently H, F, Cl, Br, I, OH, SH, or CF₃, substituted or unsubstituted C₁-C₅ alkyl, C₁-C₅ linear or branched, or C₃-C₈ cyclic haloalkyl, substituted or unsubstituted C₁-C₅ linear or branched, or C₃-C₈ cyclic alkoxy; or R₁ and R₂ are joined to form ═O or a C₃-C₈ carbocyclic or heterocyclic ring (e.g., cyclopropyl); each is a separate embodiment according to this invention;
   R₃ and R₄ are each independently H, Me, substituted or unsubstituted C₁-C₅ alkyl (e.g., methoxyethyl, methylaminoethyl, aminoethyl), —R₈—O—R₁₀ (e.g., (CH₂)₂O—CH₃), R₈—N(R₁₀)(R₁₁) (e.g., (CH₂)₂—NH(CH₃)), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀; each is a separate embodiment according to this invention;
   or R₃ and R₄ are joined to form a 3-8 membered heterocyclic ring (e.g., pyrrolidine, 2-oxopyrrolidine, piperidine, morpholine, piperazine, imidazole; each is a separate embodiment according to this invention);
   or R₂ and R₄ are joined to form a substituted or unsubstituted, saturated or unsaturated, 3-8 membered heterocyclic or carbocyclic ring (e.g., pyrrolidine, 1-methylpyrrolidine, pyrrolidin-2-one, pyridine, piperidine, tetrahydrofurane, tetrahydrothiophene, cyclopropyl, oxetane, imidazole, pyrimidine, triazole, oxadiazole, pyrazole; each is a separate embodiment according to this invention), wherein if the ring is aromatic, then R₁ and/or R₃ are absent;
   wherein if X₁ is O then R₄ is absent;

In some embodiments, X₁ of formula A is N. In other embodiments X₁ is O.

In some embodiments, R₁ of formula A is H. In other embodiments R₁ is F. In other embodiments R₁ is CF₃.

In some embodiments, R₂ of formula A is H. In other embodiments R₂ is F. In other embodiments R₂ is CF₃.

In some embodiments, R₁ and R₂ of formula A are joined to form ═O. In other embodiments, R₁ and R₂ are joined to form a C₃-C₅ carbocyclic or heterocyclic ring. In other embodiments, R₁ and R₂ are joined to form a C₃-C₅ carbocyclic ring. In some embodiments, the carbocyclic ring is cyclopropyl. In other embodiments, R₁ and R₂ are joined to form a 3-8 membered heterocyclic ring.

In some embodiments, R₁ and R₂ of formula A of formula I, I(a)-I(c), I(i) and/or I(k)-I(n), are not joined to form ═O.

In some embodiments, R₃ of formula A is H. In some embodiments, R₃ is methyl. In some embodiments, R₃ is substituted or unsubstituted C₁-C₅ alkyl. In some embodiments, the alkyl is methoxyethylene, methylaminoethylene, aminoethylene; each represents a separate embodiment according to this invention. In some embodiments, R₃ is substituted or unsubstituted C₃-C₅ cycloalkyl. In some embodiments, the cycloalkyl is cyclopropyl. In some embodiments, R₃ is substituted or unsubstituted 5-7 membered heterocyclic ring. In some embodiments, the heterocyclic ring is pyrrolidine, methylpyrrolidine, or piperidine; each represents a separate embodiment according to this invention. In some embodiments, R₃ is R₂₀ as defined hereinbelow.

In some embodiments, R₄ of formula A is H. In some embodiments, R₄ is methyl. In some embodiments, R₄ is substituted or unsubstituted C₁-C₅ alkyl. In some embodiments, the alkyl is methoxyethylene, methylaminoethylene, aminoethylene; each represents a separate embodiment according to this invention. In some embodiments, R₄ is substituted or unsubstituted C₃-C₅ cycloalkyl. In some embodiments, the cycloalkyl is cyclopropyl. In some embodiments, R₄ is substituted or unsubstituted 5-7 membered heterocyclic ring. In some embodiments, the heterocyclic ring is pyrrolidine, methylpyrrolidine, or piperidine; each represents a separate embodiment according to this invention. In some embodiments, R₄ is R₂₀ as defined hereinbelow.

In some embodiments, R₃ and R₄ of formula A are joined to form a 3-8 membered heterocyclic ring. In some embodiments, the heterocyclic ring is imidazole, pyrrolidine, 2-oxopyrrolidine, piperidine, morpholine, or piperazine; each represents a separate embodiment according to this invention.

In some embodiments, if X₁ of formula A is O then R₄ is absent.

In some embodiments, R, of formula I, I(a)-I(c), I(i), I(j), I(k) and/or I(n) is O—R₂₀, substituted or unsubstituted 4-7 membered heterocyclic ring (e.g., morpholine, pyran, oxetane, pyrrolidine, imidazole, piperazine, piperidine, dioxazole, triazole, 2-oxopyrrolidine), or substituted or unsubstituted aryl. In some embodiments, R, of formula I, I(a)-I(c), I(i), I(j), I(k) and/or I(n) is represented by formula A, wherein X₁, R₁, R₂, R₃ and R₄ are as defined above except that R₁ and R₂ cannot be joined to form ═O.

In some embodiments, R₇′ of formula I, and/or I(a)-I(n) is not H.

In some embodiments, R₇′ of formula I and/or I(a)-I(n) is H. In some embodiments, R₇′ of formula I and/or I(a)-I(n) is F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, isopropyl, methyl, ethyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, cyclopropyl, cyclohexyl, substituted or unsubstituted 3-8 membered heterocyclic ring, substituted or unsubstituted aryl, phenyl, or substituted or unsubstituted benzyl; each represents a separate embodiment according to this invention. In some embodiments, R₇′ is further substituted with at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

In some embodiments, R₇′ of formula I and/or I(a)-I(n) is H. In some embodiments, R₇′ is F. In some embodiments, R₇′ is Cl. In some embodiments, R₇′ is Br. In some embodiments, R₇′ is I. In some embodiments, R₇′ is CF₃. In some embodiments, R₇′ is C₁-C₅ linear or branched, substituted or unsubstituted alkyl. In some embodiments, R₇′ is C₁-C₅ linear or branched unsubstituted alkyl. In some embodiments, the alkyl is isopropyl, methyl, ethyl; each represents a separate embodiment according to this invention. In some embodiments, R₇′ is C₁-C₅ linear or branched substituted alkyl. In some embodiments, R₇′ is isopropyl. In some embodiments, R₇′ is methyl. In some embodiments, R₇′ is ethyl. In some embodiments, R₇′ is C₁-C₅ linear or branched, or C₃-C₈ cyclic haloalkyl. In some embodiments, R₇′ is C₁-C₅ linear or branched haloalkyl. In some embodiments, the haloalkyl is CHF₂. In some embodiments, R₇′ is C₃-C₈ cyclic haloalkyl. In some embodiments, R₇′ is substituted or unsubstituted C₃-C₈ cycloalkyl. In some embodiments, the cycloalkyl is cyclopropyl. In some embodiments, the cycloalkyl is cyclohexyl. In some embodiments, R₇′ is substituted or unsubstituted aryl. In some embodiments, R₇′ is phenyl. In some embodiments, R₇′ is C₁-C₅ linear or branched, or C₃-C₈ cyclic alkoxy. In some embodiments, R₇′ is methoxy.

In some embodiments, R₇ and R₇′ of formula I, I(a)-I(c) and/or I(i)-I(n) are joined to form a 5 or 6 membered substituted or unsubstituted, saturated, unsaturated or aromatic, carbocyclic or heterocyclic ring. In some embodiments, R₇ and R₇′ are joined to form a 5 membered unsubstituted saturated or unsaturated carbocyclic ring. In some embodiments, R₇ and R₇′ are joined to form 6 membered unsubstituted saturated or unsaturated carbocyclic ring. In some embodiments, R₇ and R₇′ are joined to form a 5 membered substituted saturated or unsaturated carbocyclic ring. In some embodiments, R₇ and R₇′ are joined to form 6 membered substituted saturated or unsaturated carbocyclic ring. In some embodiments, R₇ and R₇′ are joined to form a 6 membered substituted or unsubstituted, aromatic, carbocyclic ring. In some embodiments, R₇ and R₇′ are joined to form a 5 or 6 membered substituted or unsubstituted, aromatic, heterocyclic ring. In some embodiments, R₇ and R₇′ are joined to form a 5 or 6 membered substituted or unsubstituted, heterocyclic ring. In some embodiments, R₇ and R₇′ are joined to form a 6 membered substituted or unsubstituted, heterocyclic ring. In some embodiments, R₇ and R₇′ are joined to form a piperidine. In some embodiments, R₇ and R₇′ are joined to form a tetrahydropyran. In some embodiments, R₇ and R₇′ are joined to form a 5 membered substituted or unsubstituted, heterocyclic ring. In some embodiments, R₇ and R₇′ are joined to form a pyrrolidine. In some embodiments, R₇ and R₇′ are joined to form a tetrahydrofuran.

In some embodiments, R₇ and R₇′ of formula I, I(a)-I(c), I(i)-I(k) and/or I(n) are different. In some embodiments, R₇ and R₇′ of formula I, I(a)-I(c), I(i)-I(k) and/or I(n) are not H, F, Cl, C₁-C₅ linear or branched, or C₃-C₈ cyclic alkoxy, C₁-C₅ linear or branched haloalkoxy or C₁-C₅ linear or branched, substituted or unsubstituted alkyl; each represents a separate embodiment according to this invention.

In some embodiments, R₇″ of formula I(a), I(c), I(e), and/or I(f)-I(n) is not H.

In some embodiments, R₇″ of formula I(a), I(c), I(e), and/or I(f)-I(n) is H. In some embodiments, R₇″ of formula I(a), I(c), I(e), and/or I(f)-I(n) is F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, cyclopropyl, cyclohexyl, substituted or unsubstituted 3-8 membered heterocyclic ring, substituted or unsubstituted aryl, phenyl, or substituted or unsubstituted benzyl; each represents a separate embodiment according to this invention. In some embodiments, R₇″ is further substituted with at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

In some embodiments, R₇″ of formula I(a), I(c), I(e), and/or I(f)-I(n) is H. In some embodiments, R₇″ is F. In some embodiments, R₇″ is Cl. In some embodiments, R₇″ is Br. In some embodiments, R₇″ is I. In some embodiments, R₇″ is CF₃. In some embodiments, R₇″ is C₁-C₅ linear or branched, substituted or unsubstituted alkyl. In some embodiments, R₇″ is C₁-C₅ linear or branched unsubstituted alkyl. In some embodiments, the alkyl is isopropyl, methyl, ethyl; each represents a separate embodiment according to this invention. In some embodiments, R₇″ is C₁-C₅ linear or branched substituted alkyl. In some embodiments, R₇″ is isopropyl. In some embodiments, R₇″ is methyl. In some embodiments, R₇″ is ethyl. In some embodiments, R₇″ is C₁-C₅ linear or branched, or C₃-C₈ cyclic haloalkyl. In some embodiments, R₇″ is C₁-C₅ linear or branched haloalkyl. In some embodiments, the haloalkyl is CHF₂. In some embodiments, R₇″ is C₃-C₈ cyclic haloalkyl. In some embodiments, R₇″ is substituted or unsubstituted C₃-C₈ cycloalkyl. In some embodiments, the cycloalkyl is cyclopropyl. In some embodiments, the cycloalkyl is cyclohexyl. In some embodiments, R₇″ is substituted or unsubstituted aryl. In some embodiments, R₇″ is phenyl. In some embodiments, R₇″ is C₁-C₅ linear or branched, or C₃-C₈ cyclic alkoxy. In some embodiments, R₇″ is methoxy.

In some embodiments, R₇′″ of formula I(a), I(c), I(e), and/or I(f)-I(n) is not H.

In some embodiments, R₇′″ of formula I(a), I(c), I(e), and/or I(f)-I(n) is H. In some embodiments, R₇′″ of formula I(i) is F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, cyclopropyl, cyclohexyl, substituted or unsubstituted 3-8 membered heterocyclic ring, substituted or unsubstituted aryl, phenyl, or substituted or unsubstituted benzyl; each represents a separate embodiment according to this invention. In some embodiments, R₇′″ is cyclopropyl. In some embodiments, R₇′″ is further substituted with at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

In some embodiments, R₇′″ of formula I(a), I(c), I(e), and/or I(f)-I(n) is H. In some embodiments, R₇′″ is F. In some embodiments, R₇′″ is Cl. In some embodiments, R₇′″ is Br. In some embodiments, R₇′″ is I. In some embodiments, R₇′″ is CF₃. In some embodiments, R₇′″ is C₁-C₅ linear or branched, substituted or unsubstituted alkyl. In some embodiments, R₇′″ is C₁-C₅ linear or branched unsubstituted alkyl. In some embodiments, the alkyl is isopropyl, methyl, ethyl; each represents a separate embodiment according to this invention. In some embodiments, R₇′″ is C₁-C₅ linear or branched substituted alkyl. In some embodiments, R₇′″ is isopropyl. In some embodiments, R₇′″ is methyl. In some embodiments, R₇′″ is ethyl. In some embodiments, R₇′″ is C₁-C₅ linear or branched, or C₃-C₈ cyclic haloalkyl. In some embodiments, R₇′″ is C₁-C₅ linear or branched haloalkyl. In some embodiments, the haloalkyl is CHF₂. In some embodiments, R₇′″ is C₃-C₈ cyclic haloalkyl. In some embodiments, R₇′″ is substituted or unsubstituted C₃-C₈ cycloalkyl. In some embodiments, the cycloalkyl is cyclopropyl. In some embodiments, the cycloalkyl is cyclohexyl. In some embodiments, R₇′″ is substituted or unsubstituted aryl. In some embodiments, R₇′″ is phenyl. In some embodiments, R₇′″ is C₁-C₅ linear or branched, or C₃-C₈ cyclic alkoxy. In some embodiments, R₇′″ is methoxy.

In some embodiments, R₇″″ of formula I(a), I(c), I(e), and/or I(f)-I(n) is not H.

In some embodiments, R₇″″ of formula I(a), I(c), I(e), and/or I(f)-I(n) is H. In some embodiments, R₇″″ of formula I(i) is F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, cyclopropyl, cyclohexyl, substituted or unsubstituted 3-8 membered heterocyclic ring, substituted or unsubstituted aryl, phenyl, or substituted or unsubstituted benzyl; each represents a separate embodiment according to this invention. In some embodiments, R₇″″ is further substituted with at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

In some embodiments, R₇″″ of formula I(a), I(c), I(e), and/or I(f)-I(n) is H. In some embodiments, R₇″″ is F. In some embodiments, R₇″″ is Cl. In some embodiments, R₇″″ is Br. In some embodiments, R₇″″ is I. In some embodiments, R₇″″ is CF₃. In some embodiments, R₇″″ is C₁-C₅ linear or branched, substituted or unsubstituted alkyl. In some embodiments, R₇″″ is C₁-C₅ linear or branched unsubstituted alkyl. In some embodiments, the alkyl is isopropyl, methyl, ethyl; each represents a separate embodiment according to this invention. In some embodiments, R₇″″ is C₁-C₅ linear or branched substituted alkyl. In some embodiments, R₇″″ is isopropyl. I'n some embodiments, R₇″″ is methyl. In some embodiments, R₇″″ is ethyl. In some embodiments, R₇″″ is C₁-C₅ linear or branched, or C₃-C₅ cyclic haloalkyl. In some embodiments, R₇″″ is C₁-C₅ linear or branched haloalkyl. In some embodiments, the haloalkyl is CHF₂. In some embodiments, R₇″″ is C₃-C₅ cyclic haloalkyl. In some embodiments, R₇″″ is substituted or unsubstituted C₃-C₅ cycloalkyl. In some embodiments, the cycloalkyl is cyclopropyl. In some embodiments, the cycloalkyl is cyclohexyl. In some embodiments, R₇″″ is substituted or unsubstituted aryl. In some embodiments, R₇″″ is phenyl. In some embodiments, R₇″″ is C₁-C₅ linear or branched, or C₃-C₅ cyclic alkoxy. In some embodiments, R₇″″ is methoxy.

In some embodiments, R₇′ and R₇″ of formula I(a), I(c), I(e), and/or I(f)-I(n) are joined to form a 3-8 membered substituted or unsubstituted, saturated, unsaturated or aromatic, carbocyclic or heterocyclic ring. In some embodiments, R₇′ and R₇″ are joined to form a 5 membered unsubstituted saturated or unsaturated carbocyclic ring. In some embodiments, R₇′ and R₇″ are joined to form a cyclopentane. In some embodiments, R₇′ and R₇″ are joined to form 6 membered unsubstituted saturated or unsaturated carbocyclic ring. In some embodiments, R₇′ and R₇″ are joined to form a cyclohexane. In some embodiments, R₇′ and R₇″ are joined to form a 5 membered substituted saturated or unsaturated carbocyclic ring. In some embodiments, R₇′ and R₇″ are joined to form 6 membered substituted saturated or unsaturated carbocyclic ring. In some embodiments, R₇′ and R₇″ are joined to form a 6 membered substituted or unsubstituted, aromatic, carbocyclic ring. In some embodiments, R₇′ and R₇″ are joined to form a 5 or 6 membered substituted or unsubstituted, aromatic, heterocyclic ring. In some embodiments, R₇′ and R₇″ are joined to form a 5 or 6 membered substituted or unsubstituted, heterocyclic ring. In some embodiments, R₇′ and R₇″ are joined to form a 6 membered substituted or unsubstituted, heterocyclic ring. In some embodiments, R₇′ and R₇″ are joined to form a piperidine. In some embodiments, R₇′ and R₇″ are joined to form a tetrahydropyran. In some embodiments, R₇′ and R₇″ are joined to form a 5 membered substituted or unsubstituted, heterocyclic ring. In some embodiments, R₇′ and R₇″ are joined to form a tetrahydrofuran. In some embodiments, R₇′ and R₇″ are joined to form a pyrrolidine.

In some embodiments, R₇′ and R₇″ of formula I(a), I(c), I(e), and/or I(f)-I(n) are different. In some embodiments, R₇′ and R₇″ of formula I(a), I(c), I(e), and/or I(f)-I(n) are not H, F, Cl, C₁-C₅ linear or branched, or C₃-C₅ cyclic alkoxy, C₁-C₅ linear or branched haloalkoxy or C₁-C₅ linear or branched, substituted or unsubstituted alkyl; each represents a separate embodiment according to this invention.

In some embodiments, R₇″ and R₇ of formula I(a), I(c), I(i), I(j)-I(k) and/or I(n) are joined to form a 3-8 membered substituted or unsubstituted, saturated, unsaturated or aromatic, carbocyclic or heterocyclic ring. In some embodiments, R₇″ and R₇ are joined to form a 5 membered unsubstituted saturated or unsaturated carbocyclic ring. In some embodiments, R₇″ and R₇ are joined to form a cyclopentane. In some embodiments, R₇″ and R₇ are joined to form 6 membered unsubstituted saturated or unsaturated carbocyclic ring. In some embodiments, R₇″ and R₇ are joined to form a cyclohexane. In some embodiments, R₇″ and R₇ are joined to form a 5 membered substituted saturated or unsaturated carbocyclic ring. In some embodiments, R₇″ and R₇ are joined to form 6 membered substituted saturated or unsaturated carbocyclic ring. In some embodiments, R₇′ and R₇″ are joined to form a 6 membered substituted or unsubstituted, aromatic, carbocyclic ring. In some embodiments, R₇″ and R₇ are joined to form a 5 or 6 membered substituted or unsubstituted, aromatic, heterocyclic ring. In some embodiments, R₇″ and R₇ are joined to form a 5 or 6 membered substituted or unsubstituted, heterocyclic ring. In some embodiments, R₇″ and R₇ are joined to form a 6 membered substituted or unsubstituted, heterocyclic ring. In some embodiments, R₇″ and R₇ are joined to form a piperidine. In some embodiments, R₇″ and R₇ are joined to form a tetrahydropyran. In some embodiments, R₇″ and R₇ are joined to form a 5 membered substituted or unsubstituted, heterocyclic ring. In some embodiments, R₇″ and R₇ are joined to form a tetrahydrofuran. In some embodiments, R₇″ and R₇ are joined to form a pyrrolidine.

In some embodiments, R₇″ and R₇ of formula I(a), I(c), I(i), I(j)-I(k) and/or I(n) are different. In some embodiments, R₇″ and R₇ of I(a), I(c), I(i), I(j)-I(k) and/or I(n) are not H, F, Cl, C₁-C₅ linear or branched, or C₃-C₅ cyclic alkoxy, C₁-C₅ linear or branched haloalkoxy or C₁-C₅ linear or branched, substituted or unsubstituted alkyl; each represents a separate embodiment according to this invention.

In some embodiments, R₇ and R₇′″ of formula I(a), I(c), I(i), I(j)-I(k) and/or I(n) are joined to form a 3-8 membered substituted or unsubstituted, saturated, unsaturated or aromatic, carbocyclic or heterocyclic ring. In some embodiments, R₇ and R₇′″ are joined to form a 5 membered unsubstituted saturated or unsaturated carbocyclic ring. In some embodiments, R₇ and R₇′″ are joined to form 6 membered unsubstituted saturated or unsaturated carbocyclic ring. In some embodiments, R₇ and R₇′″ are joined to form a 5 membered substituted saturated or unsaturated carbocyclic ring. In some embodiments, R₇ and R₇′″ are joined to form 6 membered substituted saturated or unsaturated carbocyclic ring. In some embodiments, R₇ and R₇′″ are joined to form a 6 membered substituted or unsubstituted, aromatic, carbocyclic ring. In some embodiments, R₇ and R₇′″ are joined to form a 5 or 6 membered substituted or unsubstituted, aromatic, heterocyclic ring. In some embodiments, R₇ and R₇′″ are joined to form a 5 or 6 membered substituted or unsubstituted, heterocyclic ring. In some embodiments, R₇ and R₇′″ are joined to form a 6 membered substituted or unsubstituted, heterocyclic ring. In some embodiments, R₇ and R₇′″ are joined to form a piperidine. In some embodiments, R₇ and R₇′″ are joined to form a tetrahydrofuran. In some embodiments, R₇ and R₇′″ are joined to form a tetrahydropyran. In some embodiments, R₇ and R₇′″ are joined to form a 5 membered substituted or unsubstituted, heterocyclic ring. In some embodiments, R₇ and R₇′″ are joined to form a pyrrolidine. In some embodiments, R₇ and R₇′″ are joined to form a cyclopentane. In some embodiments, R₇ and R₇′″ are joined to form a cyclohexane.

In some embodiments, R₇ and R₇′″ of formula I(a), I(c), I(i), I(j)-I(k) and/or I(n) are different. In some embodiments, R₇ and R₇′″ of formula I(i) are not H, F, Cl, C₁-C₅ linear or branched, or C₃-C₈ cyclic alkoxy, C₁-C₅ linear or branched haloalkoxy or C₁-C₅ linear or branched, substituted or unsubstituted alkyl; each represents a separate embodiment according to this invention.

In some embodiments, R₇′″ and R₇″″ of formula I(a), I(c), I(i), I(j)-I(k) and/or I(n) are joined to form a 3-8 membered substituted or unsubstituted, saturated, unsaturated or aromatic, carbocyclic or heterocyclic ring. In some embodiments, R₇′″ and R₇″″ are joined to form a 5 membered unsubstituted saturated or unsaturated carbocyclic ring. In some embodiments, R₇′″ and R₇″″ are joined to form 6 membered unsubstituted saturated or unsaturated carbocyclic ring. In some embodiments, R₇′″ and R₇″″ are joined to form a 5 membered substituted saturated or unsaturated carbocyclic ring. In some embodiments, R₇″ and R₇″″ are joined to form 6 membered substituted saturated or unsaturated carbocyclic ring. In some embodiments, R₇′″ and R₇″″ are joined to form a 6 membered substituted or unsubstituted, aromatic, carbocyclic ring. In some embodiments, R₇′″ and R₇″″ are joined to form a 5 or 6 membered substituted or unsubstituted, aromatic, heterocyclic ring. In some embodiments, R₇′″ and R₇″″ are joined to form a 5 or 6 membered substituted or unsubstituted, heterocyclic ring. In some embodiments, R₇′″ and R₇″″ are joined to form a 6 membered substituted or unsubstituted, heterocyclic ring. In some embodiments, R₇′″ and R₇″″ are joined to form a piperidine. In some embodiments, R₇′″ and R₇″″ are joined to form a tetrahydrofuran. In some embodiments, R₇′″ and R₇″″ are joined to form a tetrahydropyran. In some embodiments, R₇′″ and R₇″″ are joined to form a 5 membered substituted or unsubstituted, heterocyclic ring. In some embodiments, R₇′″ and R₇″″ are joined to form a pyrrolidine. In some embodiments, R₇′″ and R₇″″ are joined to form a cyclopentane. In some embodiments, R₇′″ and R₇″″ are joined to form a cyclohexane.

In some embodiments, R₇′″ and R₇″″ of formula I(a), I(c), and/or I(e)-I(n) are different. In some embodiments, R₇′″ and R₇″″ of formula I(i) are not H, F, Cl, C₁-C₅ linear or branched, or C₃-C₈ cyclic alkoxy, C₁-C₅ linear or branched haloalkoxy or C₁-C₅ linear or branched, substituted or unsubstituted alkyl; each represents a separate embodiment according to this invention.

In some embodiments, at least one of R₇′, R₇″, R₇′″ and R₇″″ of formula I(a), I(c), and/or I(e)-I(n) is not H. In some embodiments, at least two of R₇′, R₇″, R₇′″ and R₇″″ of formula I(a), I(c), and/or I(e)-I(n) are not H. In some embodiments, at least three of R₇′, R₇″, R₇′″ and R₇″″ of formula I(a), I(c), and/or I(e)-I(n) are not H.

In some embodiments, at least one of R₇, R₇′, R₇″, R₇′″ and R₇″″ of formula I(a), I(c), I(i), I(j)-I(k) and/or I(n) is not H. In some embodiments, at least two of R₇, R₇′, R₇″, R₇′″ and R₇″″ of formula I(a), I(c), I(i), I(j)-I(k) and/or I(n) are not H. In some embodiments, at least three of R₇, R₇′, R₇″, R₇′″ and R₇″″ of formula I(a), I(c), I(i), I(j)-I(k) and/or I(n) are not H.

In some embodiments, R₃₀ of formula I and/or I(a)-I(n) is H, R₂₀, F, Cl, Br, I, OH, SH, alkoxy, N(R)₂, NH(R₁₀), N(R₁₀)(R₁₁), CF₃, CN, NO₂, C₁-C₅ linear or branched, substituted or unsubstituted alkyl, C₁-C₅ linear or branched alkoxy, C₁-C₅ linear or branched haloalkyl, R₈-aryl, —R₈—O—R₈—O—R₁₀, —R₈—O—R₁₀, —R₈—R₁₀, substituted or unsubstituted aryl, or substituted or unsubstituted heteroaryl; each represents a separate embodiment according to this invention. In some embodiments, R₃₀ is further substituted with at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₅ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention. In some embodiments, R₃₀ is H. In some embodiments, R₃₀ is R₂₀.

In some embodiments, R₅₀ of formula I(b), I(c), I(f), I(h), I(l) and/or I(m) is H, R₂₀, F, Cl, Br, I, OH, SH, N(R)₂, NH(R₁₀), N(R₁₀)(R₁₁), CF₃, CN, NO₂, C₁-C₅ linear or branched, substituted or unsubstituted alkyl C₁-C₅ linear or branched alkoxy, C₁-C₅ linear or branched haloalkyl, —R₈-R₁₀ (e.g., (CH₂)₂O—CH₃), substituted or unsubstituted aryl (e.g., phenyl), substituted or unsubstituted heteroaryl (e.g., pyridine (2, 3, and 4-pyridine); each represents a separate embodiment according to this invention.

In some embodiments, R₅₀ is further substituted with at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₅ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention. In some embodiments, R₅₀ is H. In some embodiments, R₅₀ is F. In some embodiments, R₅₀ is CF₃. In some embodiments, R₅₀ is CN.

In some embodiments, Ring G of formula I(b), I(c), I(f), I(h), I(l) and/or I(m) is absent. In some embodiments, Ring G is a substituted or unsubstituted 3-8 membered carbocyclic or heterocyclic ring. In some embodiments, Ring G is a substituted 3-8 membered carbocyclic ring. In some embodiments, Ring G is a unsubstituted 3-8 membered carbocyclic ring. In some embodiments, Ring G is a unsubstituted 4-7 membered carbocyclic ring. In some embodiments, Ring G is a unsubstituted 3-6 membered carbocyclic ring. In some embodiments, Ring G is cyclobutane. In some embodiments, Ring G is cyclopentane. In some embodiments, Ring G is cyclohexane. In some embodiments, Ring G is a substituted or unsubstituted 3-8 membered heterocyclic ring. In some embodiments, Ring G is a substituted 3-8 membered heterocyclic ring.

In some embodiments, R of formula I and/or I(a)-I(n) is H, F, Cl, Br, I, OH, SH, COOH, CO(R₁₀), C(O)CH₃, NH(R₁₀), NH—CH₂-cyclopropyl, N(R₁₀)(R₁₁), CF₃, CN, NO₂, C₁-C₅ linear or branched, substituted or unsubstituted alkyl (e.g., methyl, ethyl, iPr, CH₂-cyclopropyl, CH₂—OH, CH₂—CH₂—OH, CH₂—CH₂—O—CH₂—CH₂—O—CH₃, CH₂—O—CH₂—CH₂—O—CH₃), C₃-C₈ substituted or unsubstituted cycloalkyl, cyclopropyl, C₁-C₅ substituted or unsubstituted, linear or branched alkoxy, (e.g., methoxy, ethoxy, O—(CH₂CH₃), OCHF₂, O—(CH₂)₂O—CH₃, isopropoxy, O—(CH₂)-cyclopropyl, O—CH₂-methylcyclobutyl, O—CH₂-3-methyloxetane, O—CH(CH₃)—CH₂—O—CH₃) C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂O—CH₃), —R₈—O—R₁₀, O—R₈-R₁₀ (e.g. O—(CH₂)₂O—CH₃, O—(CH₂CH₃), O—(CH₂)-cyclopropyl), —R₈—R₁₀ (e.g., (CH₂)-cyclopropyl, (CH₂)₂O—CH₃, (CH₂)—OH, (CH₂)₂—OH, (CH₂)—COOH), substituted or unsubstituted aryl (e.g., phenyl), substituted or unsubstituted heteroaryl (e.g., pyridine (2, 3, and 4-pyridine); each represents a separate embodiment according to this invention. In some embodiments, R is further substituted with at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₅ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention. In some embodiments, R is H. In some embodiments, R is F, Cl, Br, or I. In some embodiments, R is NH(R₁₀). In some embodiments, R is NH—CH₂-cyclopropyl. In some embodiments, R is C₁-C₅ linear or branched, substituted or unsubstituted alkyl. In some embodiments, R is methyl. In some embodiments, R is ethyl. In some embodiments, R is propyl. In some embodiments, R is isopropyl. In some embodiments, R is butyl. In some embodiments, R is substituted alkyl. In some embodiments, R is CH₂—OH. In some embodiments, R is CH₂—CH₂—OH. In some embodiments, R is C₃-C₈ substituted or unsubstituted cycloalkyl. In some embodiments, R is cyclopropyl. In some embodiments, R is C₁-C₅ linear or branched alkoxy. In some embodiments, R is methoxy. In some embodiments, R is ethoxy. In some embodiments, R is propoxy. In some embodiments, R is isopropoxy. In some embodiments, R is O—(CH₂)-cyclopropyl. In some embodiments, R is O—CH₂-methylcyclobutyl. In some embodiments, R is O—CH(CH₃)—CH₂—O—CH₃. In some embodiments, R is O—(CH₂CH₃). In some embodiments, R is OCHF₂. In some embodiments, R is O—(CH₂)₂O—CH₃. In some embodiments, R is COOH. In some embodiments, R is O—R₈-R₁₀. In some embodiments, R is O—(CH₂)₂—O—CH₃. In some embodiments, R is O—(CH₂CH₃). In some embodiments, R is —R₈—R₁₀. In some embodiments, R is (CH₂)-cyclopropyl. In some embodiments, R is (CH₂)—OH. In some embodiments, R is (CH₂)₂—OH. In some embodiments, R is (CH₂)—COOH. In some embodiments, R is OH. In some embodiments, R is CO(R₁₀).

In various embodiments, each R₈ of compound of formula I and/or I(a)-I(n) is independently CH₂. In some embodiments, R₈ is CH₂CH₂. In some embodiments, R₅ is CH₂CH₂CH₂. In some embodiments, R₈ is CH₂CH₂CH₂CH₂.

In some embodiments, p of formula I and/or I(a)-I(n) is 1. In other embodiments, p is 2. In other embodiments, p is 3. In some embodiments, p is 4. In some embodiments, p is 5. In some embodiments, p is between 1 and 3. In some embodiments, p is between 1 and 5. In some embodiments, p is between 1 and 10.

In some embodiments, R₉ of formula I and/or I(a)-I(n) is C≡C. In some embodiments, R₉ is C≡C—C≡C. In some embodiments, R₉ is CH═CH. In some embodiments, R₉ is CH═CH—CH═CH.

In some embodiments, q of formula I and/or I(a)-I(n) is 2. In some embodiments, q is 4. In some embodiments, q is 6. In some embodiments, q is 8. In some embodiments, q is between 2 and 6.

In some embodiments, R₁₀ of formula I and/or I(a)-I(n) is H, OH, COOH, C₁-C₅ substituted or unsubstituted linear or branched alkyl (e.g., methyl, ethyl, CH₂-cyclopropyl, CH₂—CH₂—O—CH₃), C₃-C₈ substituted or unsubstituted cycloalkyl (e.g., cyclopropyl), C₁-C₅ substituted or unsubstituted linear or branched haloalky, CH₂CF₃, C₁-C₅ linear or branched alkoxy (e.g., O—CH₃), R₂₀, C(O)R, or S(O)₂R; each represents a separate embodiment according to this invention. In some embodiments, R₁₀ is H. In some embodiments, R₁₀ is OH In some embodiments, R₁₀ is COOH. In some embodiments, R₁₀ is C₁-C₅ substituted or unsubstituted linear or branched alkyl. In some embodiments, R₁₀ is C₁-C₅ unsubstituted linear or branched alkyl. In other embodiments, R₁₀ is CH₃. In other embodiments, R₁₀ is CH₂CH₃. In other embodiments, R₁₀ is CH₂CH₂CH₃. In some embodiments, R₁₀ is isopropyl. In some embodiments, R₁₀ is butyl. In some embodiments, R₁₀ is isobutyl. In some embodiments, R₁₀ is t-butyl. In some embodiments, R₁₀ is pentyl. In some embodiments, R₁₀ is isopentyl. In some embodiments, R₁₀ is neopentyl. In some embodiments, R₁₀ is benzyl. In some embodiments, R₁₀ is C₁-C₅ substituted linear or branched alkyl. In other embodiments, R₁₀ is CH₂—CH₂—O—CH₃. In some embodiments, R₁₀ is C₃-C₈ substituted or unsubstituted cycloalkyl. In some embodiments, R₁₀ is cyclopropyl. In other embodiments, R₁₀ is CH₂CF₃. In other embodiments, R₁₀ is C₁-C₅ substituted or unsubstituted linear or branched haloalkyl. In other embodiments, R₁₀ is C₁-C₅ linear or branched alkoxy. In other embodiments, R₁₀ is O—CH₃. In other embodiments, R₁₀ is R₂₀. In other embodiments, R₁₀ is C(O)R. In other embodiments, R₁₀ is S(O)₂R. In some embodiments, R₁₀ is further substituted with at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₅ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

In some embodiments, R₁₁ of formula I and/or I(a)-I(n) is H, OH, COOH, C₁-C₅ substituted or unsubstituted linear or branched alkyl (e.g., methyl, ethyl, CH₂—CH₂—O—CH₃, CH₂CF₃, C₁-C₅ linear or branched alkoxy (e.g., O—CH₃), C(O)R, or S(O)₂R; each represents a separate embodiment according to this invention. In some embodiments, R₁₁ is H. In some embodiments, R₁₁ is OH In some embodiments, R₁₁ is COOH. In some embodiments, R₁₁ is C₁-C₅ substituted or unsubstituted linear or branched alkyl. In some embodiments, R₁₁ is C₁-C₅ unsubstituted linear or branched alkyl. In other embodiments, R₁₁ is CH₃. In other embodiments, R₁₁ is CH₂CH₃. In other embodiments, R₁₁ is CH₂CH₂CH₃. In some embodiments, R₁₁ is isopropyl. In some embodiments, R₁₁ is butyl. In some embodiments, R₁₁ is isobutyl. In some embodiments, R₁₁ is t-butyl. In some embodiments, R₁₁ is pentyl. In some embodiments, R₁₁ is isopentyl. In some embodiments, R₁₁ is neopentyl. In some embodiments, R₁₁ is benzyl. In some embodiments, R₁₁ is C₁-C₅ substituted linear or branched alkyl. In other embodiments, R₁₁ is CH₂—CH₂—O—CH₃. In other embodiments, R₁₁ is CH₂CF₃. In other embodiments, R₁₁ is C₁-C₅ substituted or unsubstituted linear or branched haloalkyl. In other embodiments, R₁₁ is C₁-C₅ linear or branched alkoxy. In other embodiments, R₁₁ is O—CH₃. In other embodiments, R₁₁ is R₂₀. In other embodiments, R₁₁ is C(O)R. In other embodiments, R₁₁ is S(O)₂R. In some embodiments, R₁₁ is further substituted with at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

In some embodiments, R₁₀ and R₁₁ of formula I and/or I(a)-I(n) are joined to form a substituted or unsubstituted 3-8 membered heterocyclic ring. In other embodiments, R₁₀ and R₁₁ are joined to form a piperazine ring. In other embodiments, R₁₀ and R₁₁ are joined to form a piperidine ring. In some embodiments, substitutions include: F, Cl, Br, I, C₁-C₅ linear or branched alkyl, OH, alkoxy, OMe, amide, C(O)N(R)₂, C(O)-pyrrolidine, C(O)-piperidine, N(R)₂, NH(R₁₀), N(R₁₀)(R₁₁), N(CH₃)₂, NH₂, CF₃, aryl, phenyl, heteroaryl, substituted or unsubstituted C₃-C₅ cycloalkyl, cyclobutanol, substituted or unsubstituted 3-8 membered heterocyclic ring pyran, oxetane, piperidine, pyrazole, methyl-pyrrazole, triazole, imidazole, halophenyl, (benzyloxy)phenyl, CN, and NO₂; each represents a separate embodiment according to this invention.

In some embodiments, n of formula I, I(b) and/or I(d) is an integer between 0 and 4. In some embodiments, n of formula I, I(b) and/or I(d) is an integer between 1 and 4. In some embodiments, n of formula I, I(b) and/or I(d) is 0. In some embodiments, n of formula I, I(b) and/or I(d) is 1. In some embodiments, n of formula I, I(b) and/or I(d) is 2. In some embodiments, n of formula I, I(b) and/or I(d) is 3. In some embodiments, n of formula I, I(b) and/or I(d) is 4. In some embodiments, n of formula I, I(b) and/or I(d) is 1 or 2.

In some embodiments, R₁ of formula I(d)-I(h), I(l)-I(m) and/or A is H. In other embodiments R₁ is F. In other embodiments R₁ is CF₃. In other embodiments R₁ is Cl. In other embodiments R₁ is Br. In other embodiments R₁ is I. In other embodiments R₁ is OH. In other embodiments R₁ is SH. In other embodiments R₁ is substituted or unsubstituted C₁-C₅ alkyl. In other embodiments R₁ is C₁-C₅ linear or branched, or C₃-C₅ cyclic haloalkyl. In other embodiments R₁ is substituted or unsubstituted C₁-C₅ linear or branched, or C₃-C₅ cyclic alkoxy.

In some embodiments, R₂ of formula I(d)-I(h), I(l)-I(m) and/or A is H. In other embodiments R₂ is F. In other embodiments R₂ is CF₃. In other embodiments R₂ is Cl. In other embodiments R₂ is Br. In other embodiments R₂ is I. In other embodiments R₂ is OH. In other embodiments R₂ is SH. In other embodiments R₂ is substituted or unsubstituted C₁-C₅ alkyl. In other embodiments R₂ is C₁-C₅ linear or branched, or C₃-C₅ cyclic haloalkyl. In other embodiments R₂ is substituted or unsubstituted C₁-C₅ linear or branched, or C₃-C₅ cyclic alkoxy.

In some embodiments, R₁ and R₂ of formula I(d)-I(h), I(l)-I(m) and/or A are joined to form C═O. In other embodiments, R₁ and R₂ are joined to form a 3-8 membered carbocyclic or heterocyclic ring. In other embodiments, R₁ and R₂ are joined to form a 3-8 membered carbocyclic ring. In some embodiments, the carbocyclic ring is cyclopropyl. In other embodiments, R₁ and R₂ are joined to form a 3-8 membered heterocyclic ring. In some embodiments, the heterocyclic ring is oxetane. In some embodiments, if R₁ and R₂ are joined to form a C═O, then at least one of X₂, X₃, X₄, and X₁₀ is not CH.

In some embodiments, R₃ of formula I(d)-I(h), I(l)-I(m) and/or A is H. In some embodiments, R₃ is methyl. In some embodiments, R₃ is substituted or unsubstituted C₁-C₅ alkyl. In some embodiments, the alkyl is methoxyethylene, methylaminoethylene, aminoethylene; each represents a separate embodiment according to this invention. In some embodiments, R₃ is —R₈—O—R₁₀. In some embodiments, R₃ is (CH₂)₂—O—CH₃. In some embodiments, R₃ is R₈—N(R₁₀)(R₁₁). In some embodiments, R₃ is (CH₂)₂—NH(CH₃)). In some embodiments, R₃ is substituted or unsubstituted C₃-C₈ cycloalkyl. In some embodiments, the cycloalkyl is cyclopropyl. In some embodiments, R₃ is substituted or unsubstituted 5-7 membered heterocyclic ring. In some embodiments, R₃ is pyrrolidine. In some embodiments, R₃ is methylpyrrolidine. In some embodiments, R₃ is piperidine. In some embodiments, R₃ is R₂₀ as defined hereinbelow.

In some embodiments, R₄ of formula I(d)-I(h), I(l)-I(m) and/or A is H. In some embodiments, R₄ is methyl. In some embodiments, R₄ is substituted or unsubstituted C₁-C₅ alkyl. In some embodiments, the alkyl is methoxyethylene, methylaminoethylene, aminoethylene; each represents a separate embodiment according to this invention. In some embodiments, R₄ is —R₈—O—R₁₀. In some embodiments, R₄ is (CH₂)₂—O—CH₃. In some embodiments, R₄ is R₈—N(R₁₀)(R₁₁). In some embodiments, R₄ is (CH₂)₂—NH(CH₃)). In some embodiments, R₄ is substituted or unsubstituted C₃-C₈ cycloalkyl. In some embodiments, the cycloalkyl is cyclopropyl. In some embodiments, R₄ is substituted or unsubstituted 5-7 membered heterocyclic ring. In some embodiments, R₄ is pyrrolidine. In some embodiments, R₄ is methylpyrrolidine. In some embodiments, R₄ is piperidine. In some embodiments, R₄ is R₂₀ as defined hereinbelow.

In some embodiments, R₂ and R₄ of formula I(d)-I(h), I(l)-I(m) and/or A are joined to form Ring F as defined hereinbelow. In some embodiments, R₂ and R₄ are joined to form a substituted or unsubstituted, saturated or unsaturated, 4-8 membered heterocyclic or carbocyclic ring. In some embodiments, R₂ and R₄ are joined to form a substituted or unsubstituted, unsaturated, 4-8 membered heterocyclic ring. In some embodiments, R₂ and R₄ are joined to form pyrrolidine, 1-methylpyrrolidine, pyrrolidin-2-one, pyrrolidin-3-ol, morpholine, pyridine, piperidine, tetrahydrofurane, tetrahydrothiophene, cyclopropyl, oxetane, imidazole, pyrimidine, triazole, oxadiazole, pyrazole; each represents a separate embodiment according to this invention. In some embodiments, R₂ and R₄ are joined to form pyrrolidine. In some embodiments, R₂ and R₄ are joined to form 1-methylpyrrolidine. In some embodiments, R₂ and R₄ are joined to form pyrrolidin-2-one. In some embodiments, R₂ and R₄ are joined to form pyrrolidin-3-ol. In some embodiments, R₂ and R₄ are joined to form morpholine. In some embodiments, R₂ and R₄ are joined to form piperidine. In some embodiments, if Ring F is aromatic, then R₁ is absent. In some embodiments, if Ring F is aromatic, then R₃ is absent. In some embodiments, if Ring F is aromatic, then R₁ and/or R₃ are absent.

In some embodiments, R₃ and R₄ of formula I(d)-I(h), I(l)-I(m) and/or A are joined to form a 3-8 membered heterocyclic ring. In some embodiments, the heterocyclic ring is pyrrolidine, pyrrolidone, 2-oxopyrrolidine, piperidine, morpholine, piperazine, imidazole; each represents a separate embodiment according to this invention.

In some embodiments, R₁ and R₂ of formula I(d)-I(h), I(l)-I(m) and/or A are joined to form a 3-8 membered carbocyclic or heterocyclic ring. In some embodiments, R₁ and R₂ are joined to form a cyclopropyl ring. In some embodiments, R₁ and R₂ are joined to form an oxetane ring.

In some embodiments, Ring F of formula I(d)-I(h), I(l)-I(m) and/or A is absent. In some embodiments, Ring F is a substituted or unsubstituted, saturated or unsaturated, 4-8 membered heterocyclic ring. In some embodiments, Ring F is a substituted, saturated, 4-8 membered heterocyclic ring. In some embodiments, Ring F is a substituted unsaturated, 4-8 membered heterocyclic ring. In some embodiments, Ring F is an unsubstituted, saturated, 4-8 membered heterocyclic ring. In some embodiments, Ring F is an unsubstituted, unsaturated, 4-8 membered heterocyclic ring. In some embodiments, Ring F is pyrrolidine. In some embodiments, Ring F is 1-methylpyrrolidine. In some embodiments, Ring F is pyrrolidine-2-one. In some embodiments, Ring F is pyrrolidin-3-ol. In some embodiments, Ring F is morpholine. In some embodiments, Ring F is piperidine. In some embodiments, Ring F is tetrahydrofurane. In some embodiments, Ring F is tetrahydrothiophene. In some embodiments, Ring F is cyclopropyl. In some embodiments, Ring F is oxetane. In some embodiments, Ring F is piperazine. In some embodiments, Ring F is morpholine. In some embodiments, Ring F is a pyridinyl. In other embodiments, Ring F is 2-pyridinyl. In other embodiments, Ring F is pyrimidine. In other embodiments, Ring F is imidazole. In other embodiments, Ring F is pyridazine. In other embodiments, Ring F is pyrazine. In other embodiments, Ring F is pyrazole. In other embodiments, Ring F is thiazole. In other embodiments, Ring F is isothiazolyl. In other embodiments, Ring F is thiadiazolyl. In other embodiments, Ring F is triazolyl. In other embodiments, Ring F is thiazolyl. In other embodiments, Ring F is oxazolyl. In other embodiments, Ring F is isoxazolyl. In other embodiments, Ring F is pyrrolyl. In other embodiments, Ring F is oxadiazolyl. In other embodiments, Ring F is 1,2,3-, 1,2,4-, 1,2,5- or 1,3,4-oxadiazolyl; each is a separate embodiment according to this invention. In other embodiments, Ring F is oxazolonyl. In other embodiments, Ring F is oxazolidonyl. In other embodiments, Ring F is thiazolonyl. In other embodiments, Ring F is isothiazolinonyl. In other embodiments, Ring F is isoxazolidinonyl. In other embodiments, Ring F is imidazolidinonyl. In other embodiments, Ring F is pyrazolonyl. In other embodiments, Ring F is 2H-pyrrol-2-onyl. In other embodiments, Ring F is triazolopyrimidine. In other embodiments, Ring F is 3H-[1,2,3]triazolo[4,5-d]pyrimidine, 1H-[1,2,3]triazolo[4,5-d]pyrimidine, triazolo[4,3-c]pyrimidine, [1,2,4]triazolo[4,3-a]pyrimidine, [1,2,3]triazolo[1,5-a]pyrimidine, triazolo[1,5-c]pyrimidine, [1,2,4]triazolo[1,5-a]pyrimidine or [1,2,4]triazolo[1,5-c]pyrimidine; each is a separate embodiment according to this invention. In other embodiments, Ring F is 6,7-dihydro-5H-pyrazolo[5,1-b][1,3]oxazine.

In some embodiments, Ring W of formula I(k)-I(m) may be either aromatic or non-aromatic ring. In some embodiments, Ring W is aromatic. In some embodiments, if Ring W is aromatic, then X₂, X₃, and X₄, are each independently CH, C(R) or N; each represent a separate embodiment according to this invention. In some embodiments, if Ring W is aromatic, then X₂, X₃, and X₄, are each independently C(CH₃), C(O—CH₂-cyclopropyl), C(O—CH₂-methylcyclobutyl), C(NH—CH₂-cyclopropyl), C(isopropoxy), C(O—CH(CH₃)—CH₂—O—CH₃), C(CH₂CH₃), C-iPr, C—CH₂-cyclopropyl, C(OCH₃), C(OCH₂CH₃), C(O—(CH₂)₂O—CH₃, C(OCHF₂), C(Cl), C(C(O)CH₃), C(O—CH₂CH₂—O—CH₃), or C(OH); each represent a separate embodiment according to this invention. In some embodiments, if Ring W is aromatic, then X₁₅ is C. In some embodiments, if Ring W is aromatic, then X₂, X₃, and X₄, are each independently C(CH₃), C(O—CH₂-cyclopropyl), C(O—CH₂-methylcyclobutyl), C(NH—CH₂-cyclopropyl), C(isopropoxy), C(O—CH(CH₃)—CH₂—O—CH₃), C(CH₂CH₃), C-iPr, C—CH₂-cyclopropyl, C(OCH₃), C(OCH₂CH₃), C(O—(CH₂)₂O—CH₃, C(OCHF₂), C(Cl), C(C(O)CH₃), C(O—CH₂CH₂—O—CH₃), or C(OH); each represent a separate embodiment according to this invention and X₁₅ is C.

In some embodiments, Ring W is non-aromatic. In some embodiments, Ring W is a saturated ring. In some embodiments, Ring W is unsaturated, non-aromatic ring. In some embodiments, if Ring W is non-aromatic, then X₂, X₃, and X₄, are each independently CH₂, CH(R), C(R)₂, NH, N(R), O, S, S═O or SO₂; each represents a separate embodiment according to this invention. In some embodiments, if Ring W is non-aromatic, then X₁₅ is CH, C(R) or N; each represents a separate embodiment according to this invention. In some embodiments, if Ring W is non-aromatic, then X₂, X₃, and X₄, are each independently CH₂, CH(R), C(R)₂, NH, N(R), O, S, S═O or SO₂; each represents a separate embodiment according to this invention, and X₁₅ is CH, C(R) or N; each represents a separate embodiment according to this invention.

In some embodiments, Ring W is non-aromatic, and Ring W′ is aromatic. In some embodiments, Ring W is non-aromatic, and Ring W′ is non-aromatic. In some embodiments, Ring W is aromatic, and Ring W′ is non-aromatic. In some embodiments, Ring W is aromatic, and Ring W′ is aromatic. In some embodiments, if both Ring W and Ring W′ are aromatic, then at least one of X₂, X₃, and X₄ is C(R); X₁₁ is N; or X₁₂ is not S.

In some embodiments, Ring W′ of formula I(k)-I(m) may be either aromatic or non-aromatic ring. In some embodiments, Ring W′ is aromatic. In some embodiments, if Ring W′ is aromatic, then X₁₂ is S, SO₂, O, NH, N(R), N—OH, CH═CH, CH═CH(R), C(R)═CH, N═CH, N═C(R), CH═N or C(R)═N; each represents a separate embodiment according to this invention. In some embodiments, if Ring W′ is aromatic, then X₁₂ is N—CH₂—COOH, N—CH₂—CH₂—OH, N—CH₃, N—CH₂CH₃, N-iPr, N-cyclopropyl, N—CH₂-cyclopropyl; each represents a separate embodiment according to this invention. In some embodiments, if Ring W′ is aromatic, then X₁₂ is SO₂. In some embodiments, if Ring W′ is aromatic, then X₁ is O. In some embodiments, if Ring W′ is aromatic, then X₁₂ is NH. In some embodiments, if Ring W′ is aromatic, then X₁₂ is N(R). In some embodiments, if Ring W′ is aromatic, then X₂ is N—CH₃. In some embodiments, if Ring W′ is aromatic, then X₁₂ is N—CH₂CH₃. In some embodiments, if Ring W′ is aromatic, then X₁₂ is N-iPr. In some embodiments, if Ring W′ is aromatic, then X₁₂ is N-cyclopropyl. In some embodiments, if Ring W′ is aromatic, then X₁₂ is N—CH₂-cyclopropyl. In some embodiments, if Ring W′ is aromatic, then X₁₂ is CH═CH. In some embodiments, if Ring W′ is aromatic, then X₁₂ is CH═CH(R). In some embodiments, if Ring W′ is aromatic, then X₁₂ is C(R)═CH.

In some embodiments, Ring W is non-aromatic. In some embodiments, Ring Wis a saturated ring. In some embodiments, Ring W′ is unsaturated, non-aromatic ring. In some embodiments, if Ring W′ is non-aromatic, then X₁₂ is CH═CH, CH═CH(R), C(R)═CH, OCH₂, CH₂O, SCH₂, CH₂S, CH═N, C(R)═N, N═CH, N═C(R); each represents a separate embodiment according to this invention. In some embodiments, if Ring W′ is non-aromatic, then X₁y is CH═CH. In some embodiments, if Ring W′ is non-aromatic, then X₁₂ is OCH₂. In some embodiments, if Ring W′ is non-aromatic, then X₁₂ is CH═N. In some embodiments, if Ring W′ is non-aromatic, then X₁₂ is N═CH.

In various embodiments, this invention is directed to the compounds presented in Table 1, pharmaceutical compositions and/or method of use thereof, each represents a separate embodiment according to this invention:

TABLE 1

| Comp. | | |
|---|---|---|
| No. | Structure | Compound Name |
| 100R | 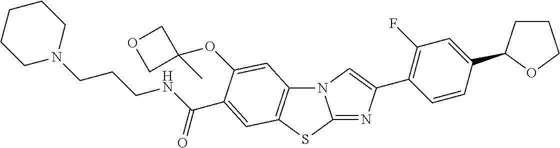 [SMILES: CC1(Oc2cc3c(cc2C(=O)NCCCN2CCCCC2)sc2nc(-c4ccc([C@H]5CCCO5)cc4F)cn23)COC1] | (R)-2-(2-fluoro-4-(tetrahydrofuran-2- yl)phenyl)-6-((3-methyloxetan-3- yl)oxy)-N-(3-(piperidin-1- yl)propyl)benzo[[d]imidazo[2,1- b]thiazole-7-carboxamide |
| 100S | 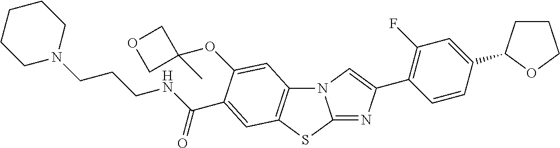 [SMILES: CC1(Oc2cc3c(cc2C(=O)NCCCN2CCCCC2)sc2nc(-c4ccc([C@@H]5CCCO5)cc4F)cn23)COC1] | (S)-2-(2-fluoro-4-(tetrahydrofuran-2- yl)phenyl)-6-((3-methyloxetan-3- yl)oxy)-N-(3-(piperidin-1- yl)propyl)benzo[d]imidazo[2,1- b]thiazole-7-carboxamide |
| 101R | 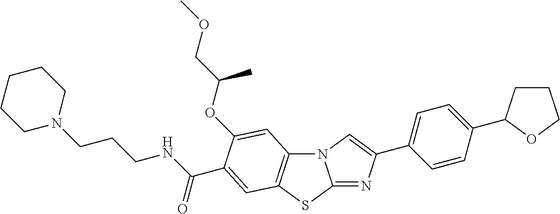 [SMILES: COC[C@@H](C)Oc1cc2c(cc1C(=O)NCCCN1CCCCC1)sc1nc(-c3ccc(C4CCCO4)cc3)cn12] | 6-(((R)-1-methoxypropan-2-yl)oxy)- N-(3-(piperidin-1-yl)propyl)-2-(4- (tetrahydrofuran-2- yl)phenyl)benzo[d]imidazo[2,1- b]thiazole-7-carboxamide |
| 101S | 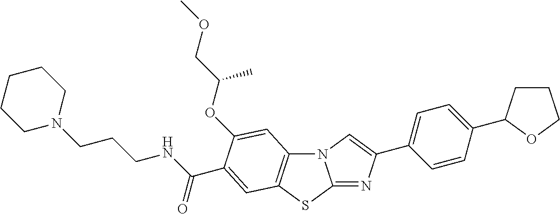 [SMILES: COC[C@H](C)Oc1cc2c(cc1C(=O)NCCCN1CCCCC1)sc1nc(-c3ccc(C4CCCO4)cc3)cn12] | 6-(((S)-1-methoxypropan-2-yl)oxy)- N-(3-(piperidin-1-yl)propyl)-2-(4- (tetrahydrofuran-2- yl)phenyl)benzo[d]imidazo[2,1- b]thiazole-7-carboxamide |
| 102S | 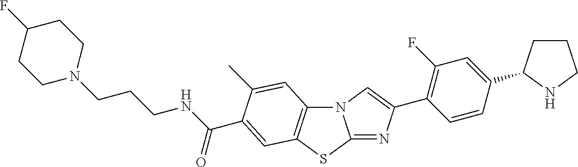 [SMILES: Cc1cc2c(cc1C(=O)NCCCN1CCC(F)CC1)sc1nc(-c3ccc([C@@H]4CCCN4)cc3F)cn12] | (S)-2-(2-fluoro-4-(pyrrolidin-2- yl)phenyl)-N-(3-(4-fluoropiperidin-1- yl)propyl)-6- methylbenzo[d]imidazo[2,1- b]thiazole-7-carboxamide |
| 102R | 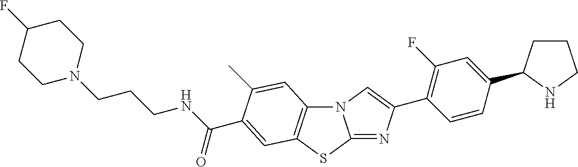 [SMILES: Cc1cc2c(cc1C(=O)NCCCN1CCC(F)CC1)sc1nc(-c3ccc([C@H]4CCCN4)cc3F)cn12] | (R)-2-(2-fluoro-4-(pyrrolidin-2- yl)phenyl)-N-(3-(4-fluoropiperidin-1- yl)propyl)-6- methylbenzo[d]imidazo[2,1- b]thiazole-7-carboxamide |
| 103S | 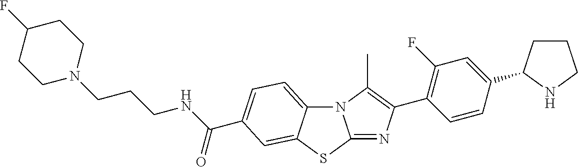 [SMILES: Cc1c(-c2ccc([C@@H]3CCCN3)cc2F)nc2sc3cc(C(=O)NCCCN4CCC(F)CC4)ccc3n12] | (S)-2-(2-fluoro-4-(pyrrolidin-2- yl)phenyl)-N-(3-(4-fluoropiperidin-1- yl)propyl)-3- methylbenzo[d]imidazo[2,1- b]thiazole-7-carboxamide |
| 103R | 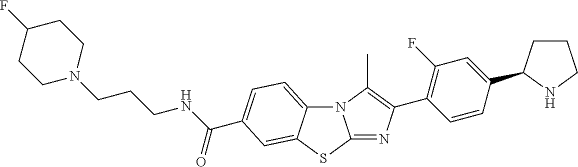 [SMILES: Cc1c(-c2ccc([C@H]3CCCN3)cc2F)nc2sc3cc(C(=O)NCCCN4CCC(F)CC4)ccc3n12] | (R)-2-(2-fluoro-4-(pyrrolidin-2- yl)phenyl)-N-(3-(4-fluoropiperidin-1- yl)propyl)-3- methylbenzo[d]imidazo[2,1- b]thiazole-7-carboxamide |
| 104S | 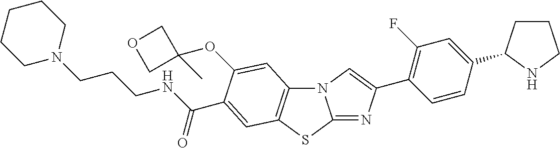 [SMILES: CC1(Oc2cc3c(cc2C(=O)NCCCN2CCCCC2)sc2nc(-c4ccc([C@@H]5CCCN5)cc4F)cn23)COC1] | (S)-2-(2-fluoro-4-(pyrrolidin-2- yl)phenyl)-6-((3-methyloxetan-3- yl)oxy)-N-(3-(piperidin-1- yl)propyl)benzo[d]imidazo[2,1- b]thiazole-7-carboxamide |
| 104R | 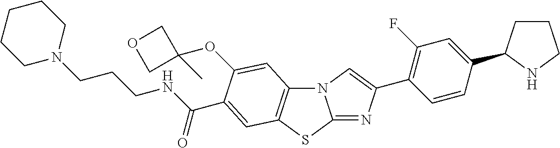 [SMILES: CC1(Oc2cc3c(cc2C(=O)NCCCN2CCCCC2)sc2nc(-c4ccc([C@H]5CCCN5)cc4F)cn23)COC1] | (R)-2-(2-fluoro-4-(pyrrolidin-2- yl)phenyl)-6-((3-methyloxetan-3- yl)oxy)-N-(3-(piperidin-1- yl)propyl)benzo[d]imidazo[2,1- b]thiazole-7-carboxamide |
| 105R | 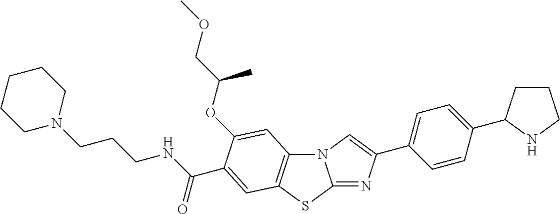 [SMILES: COC[C@@H](C)Oc1cc2c(cc1C(=O)NCCCN1CCCCC1)sc1nc(-c3ccc(C4CCCN4)cc3)cn12] | 6-(((R)-1-methoxypropan-2-yl)oxy)- N-(3-(piperidin-1-yl)propyl)-2-(4- (pyrrolidin-2- yl)phenyl)benzo[d]imidazo[2,1- b]thiazole-7-carboxamide |
| 105S | 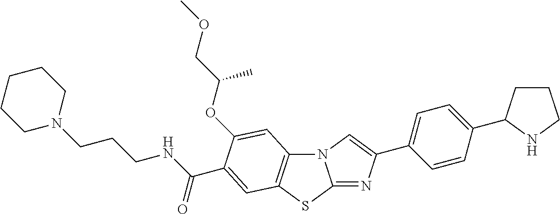 [SMILES: COC[C@H](C)Oc1cc2c(cc1C(=O)NCCCN1CCCCC1)sc1nc(-c3ccc(C4CCCN4)cc3)cn12] | 6-(((S)-1-methoxypropan-2-yl)oxy)- N-(3-(piperidin-1-yl)propyl)-2-(4- (pyrrolidin-2- yl)phenyl)benzo[d]imidazo[2,1- b]thiazole-7-carboxamide |
| 106S | 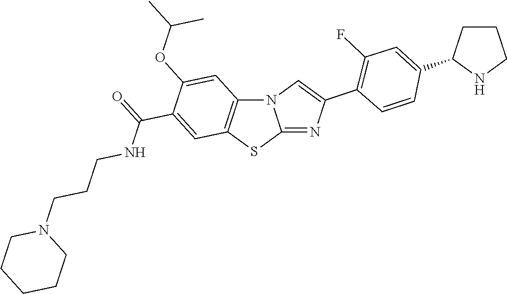 [SMILES: CC(C)Oc1cc2c(cc1C(=O)NCCCN1CCCCC1)sc1nc(-c3ccc([C@@H]4CCCN4)cc3F)cn12] | (S)-2-(2-fluoro-4-(pyrrolidin-2- yl)phenyl)-6-isopropoxy-N-(3- (piperidin-1- yl)propyl)benzo[d]imidazo[2,1- b]thiazole-7-carboxamide |
| 106R | 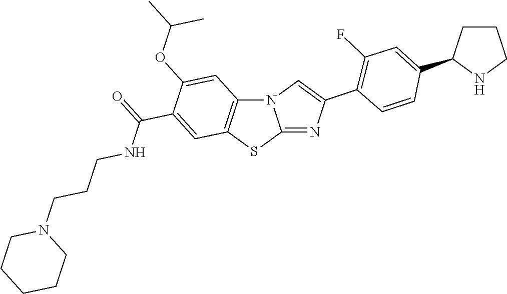 [SMILES: CC(C)Oc1cc2c(cc1C(=O)NCCCN1CCCCC1)sc1nc(-c3ccc([C@H]4CCCN4)cc3F)cn12] | (R)-2-(2-fluoro-4-(pyrrolidin-2- yl)phenyl)-6-isopropoxy-N-(3- (piperidin-1- yl)propyl)benzo[d]imidazo[2,1- b]thiazole-7-carboxamide |
| 107S | 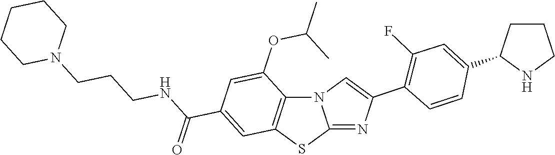 [SMILES: CC(C)Oc1cc(C(=O)NCCCN2CCCCC2)cc2sc3nc(-c4ccc([C@@H]5CCCN5)cc4F)cn3c12] | (S)-2-(2-fluoro-4-(pyrrolidin-2- yl)phenyl)-5-isopropoxy-N-(3- (piperidin-1- yl)propyl)benzo[d]imidazo[2,1- b]thiazole-7-carboxamide |
| 107R | 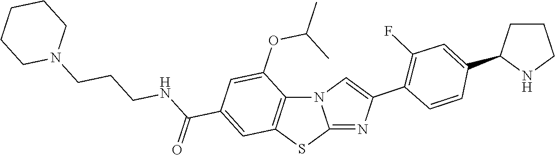 [SMILES: CC(C)Oc1cc(C(=O)NCCCN2CCCCC2)cc2sc3nc(-c4ccc([C@H]5CCCN5)cc4F)cn3c12] | (R)-2-(2-fluoro-4-(pyrrolidin-2- yl)phenyl)-5-isopropoxy-N-(3- (piperidin-1- yl)propyl)benzo[d]imidazo[2,1- b]thiazole-7-carboxamide |
| 108S | 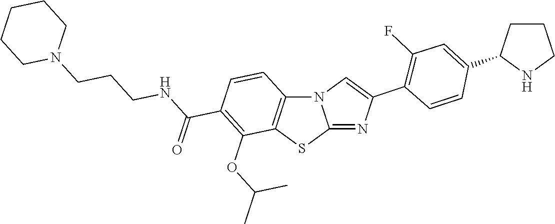 [SMILES: CC(C)Oc1c(C(=O)NCCCN2CCCCC2)ccc2c1sc1nc(-c3ccc([C@@H]4CCCN4)cc3F)cn12] | (S)-2-(2-fluoro-4-(pyrrolidin-2- yl)phenyl)-8-isopropoxy-N-(3- (piperidin-1- yl)propyl)benzo[d]imidazo[2,1- b]thiazole-7-carboxamide |
| 108R | 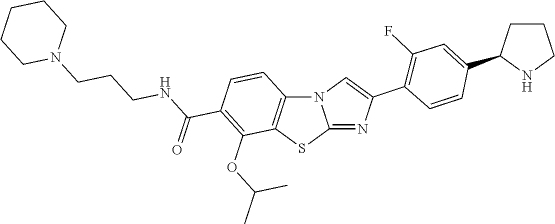 [SMILES: CC(C)Oc1c(C(=O)NCCCN2CCCCC2)ccc2c1sc1nc(-c3ccc([C@H]4CCCN4)cc3F)cn12] | (R)-2-(2-fluoro-4-(pyrrolidin-2- yl)phenyl)-8-isopropoxy-N-(3- (piperidin-1- yl)propyl)benzo[d]imidazo[2,1- b]thiazole-7-carboxamide |
| 109S | 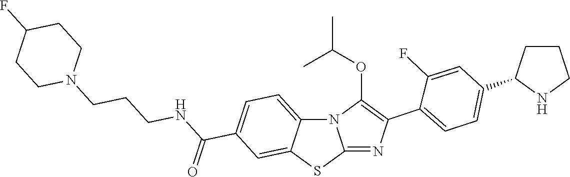 [SMILES: CC(C)Oc1c(-c2ccc([C@@H]3CCCN3)cc2F)nc2sc3cc(C(=O)NCCCN4CCC(F)CC4)ccc3n12] | (S)-2-(2-fluoro-4-(pyrrolidin-2- yl)phenyl)-N-(3-(4-fluoropiperidin-1- yl)propyl)-3- isopropoxybenzo[d]imidazo[2,1- b]thiazole-7-carboxamide |
| 109R | 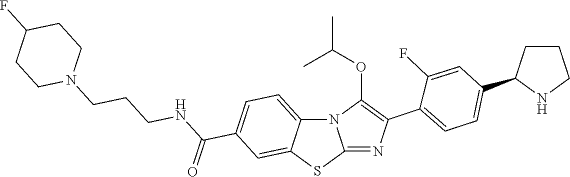 [SMILES: CC(C)Oc1c(-c2ccc([C@H]3CCCN3)cc2F)nc2sc3cc(C(=O)NCCCN4CCC(F)CC4)ccc3n12] | (R)-2-(2-fluoro-4-(pyrrolidin-2- yl)phenyl)-N-(3-(4-fluoropiperidin-1- yl)propyl)-3- isopropoxybenzo[d]imidazo[2,1- b]thiazole-7-carboxamide |
| 110S | 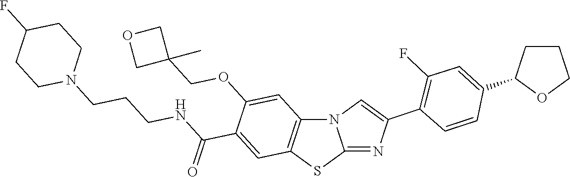 [SMILES: CC1(COc2cc3c(cc2C(=O)NCCCN2CCC(F)CC2)sc2nc(-c4ccc([C@@H]5CCCO5)cc4F)cn23)COC1] | (S)-2-(2-fluoro-4-(tetrahydrofuran-2- yl)phenyl)-N-(3-(4-fluoropiperidin-1- yl)propyl)-6-((3-methyloxetan-3- yl)methoxy)benzo[d]imidazo[2,1- b]thiazole-7-carboxamide |
| 110R | 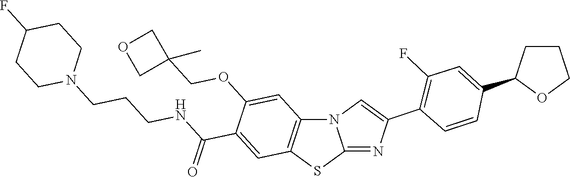 [SMILES: CC1(COc2cc3c(cc2C(=O)NCCCN2CCC(F)CC2)sc2nc(-c4ccc([C@H]5CCCO5)cc4F)cn23)COC1] | (R)-2-(2-fluoro-4-(tetrahydrofuran-2- yl)phenyl)-N-(3-(4-fluoropiperidin-1- yl)propyl)-6-((3-methyloxetan-3- yl)methoxy)benzo[d]imidazo[2,1- b]thiazole-7-carboxamide |
| 111S | 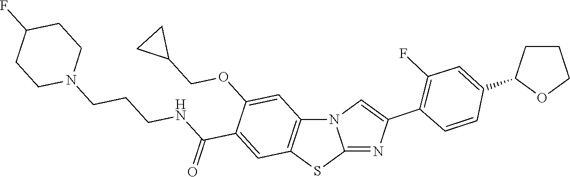 [SMILES: O=C(NCCCN1CCC(F)CC1)c1cc2sc3nc(-c4ccc([C@@H]5CCCO5)cc4F)cn3c2cc1OCC1CC1] | (S)-6-(cyclopropylmethoxy)-2-(2- fluoro-4-(tetrahydrofuran-2- yl)phenyl)-N-(3-(4-fluoropiperidin-1- yl)propyl)benzo[d]imidazo[2,1- b]thiazole-7-carboxamide |
| 111R | 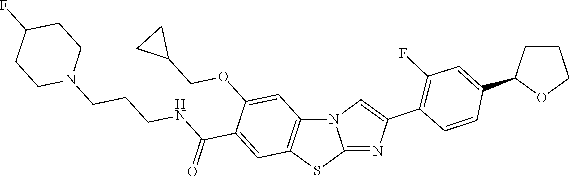 [SMILES: O=C(NCCCN1CCC(F)CC1)c1cc2sc3nc(-c4ccc([C@H]5CCCO5)cc4F)cn3c2cc1OCC1CC1] | (R)-6-(cyclopropylmethoxy)-2-(2- fluoro-4-(tetrahydrofuran-2- yl)phenyl)-N-(3-(4-fluoropiperidin-1- yl)propyl)benzo[d]imidazo[2,1- b]thiazole-7-carboxamide |
| 112 | 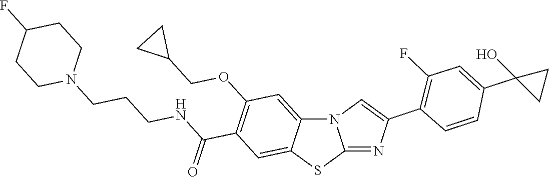 [SMILES: O=C(NCCCN1CCC(F)CC1)c1cc2sc3nc(-c4ccc(C5(O)CC5)cc4F)cn3c2cc1OCC1CC1] | 6-(cyclopropylmethoxy)-2-(2-fluoro- 4-(1-hydroxycyclopropyl)phenyl)-N- (3-(4-fluoropiperidin-1- yl)propyl)benzo[d]imidazo[2,1- b]thiazole-7-carboxamide |
| 113 | 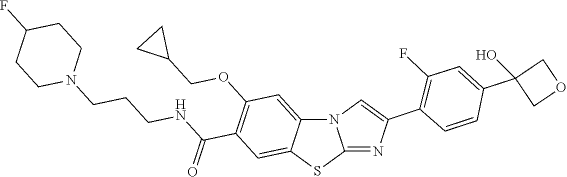 [SMILES: O=C(NCCCN1CCC(F)CC1)c1cc2sc3nc(-c4ccc(C5(O)COC5)cc4F)cn3c2cc1OCC1CC1] | 6-(cyclopropylmethoxy)-2-(2-fluoro- 4-(3-hydroxyoxetan-3-yl)phenyl)-N- (3-(4-fluoropiperidin-1- yl)propyl)benzo[d]imidazo[2,1- b]thiazole-7-carboxamide |
| 114S | 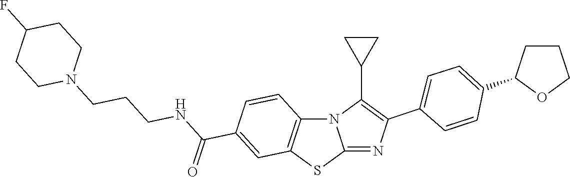 [SMILES: O=C(NCCCN1CCC(F)CC1)c1ccc2c(c1)sc1nc(-c3ccc([C@@H]4CCCO4)cc3)c(C3CC3)n12] | (S)-3-cyclopropyl-N-(3-(4- fluoropiperidin-1-yl)propyl)-2-(4- (tetrahydrofuran-2- yl)phenyl)benzo[d]imidazo[2,1- b]thiazole-7-carboxamide |
| 114R | 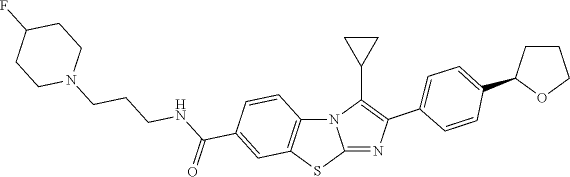 [SMILES: O=C(NCCCN1CCC(F)CC1)c1ccc2c(c1)sc1nc(-c3ccc([C@H]4CCCO4)cc3)c(C3CC3)n12] | (R)-3-cyclopropyl-N-(3-(4- fluoropiperidin-1-yl)propyl)-2-(4- (tetrahydrofuran-2- yl)phenyl)benzo[d]imidazo[2,1- b]thiazole-7-carboxamide |
| 115S | 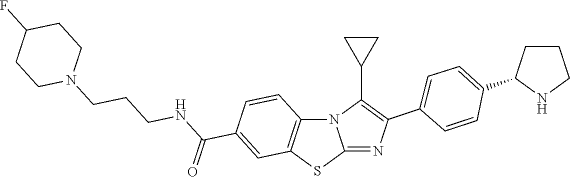 [SMILES: O=C(NCCCN1CCC(F)CC1)c1ccc2c(c1)sc1nc(-c3ccc([C@@H]4CCCN4)cc3)c(C3CC3)n12] | (S)-3-cyclopropyl-N-(3-(4- fluoropiperidin-1-yl)propyl)-2-(4- (pyrrolidin-2- yl)phenyl)benzo[d]imidazo[2,1- b]thiazole-7-carboxamide |
| 115R | 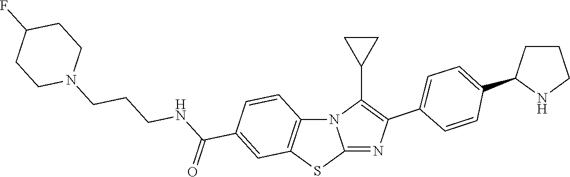 [SMILES: O=C(NCCCN1CCC(F)CC1)c1ccc2c(c1)sc1nc(-c3ccc([C@H]4CCCN4)cc3)c(C3CC3)n12] | (R)-3-cyclopropyl-N-(3-(4- fluoropiperidin-1-yl)propyl)-2-(4- (pyrrolidin-2- yl)phenyl)benzo[d]imidazo[2,1- b]thiazole-7-carboxamide |
| 116S | 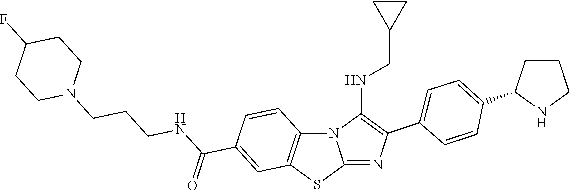 [SMILES: O=C(NCCCN1CCC(F)CC1)c1ccc2c(c1)sc1nc(-c3ccc([C@@H]4CCCN4)cc3)c(NCC3CC3)n12] | (S)-3-((cyclopropylmethyl)amino)-N- (3-(4-fluoropiperidin-1-yl)propyl)-2- (4-(pyrrolidin-2- yl)phenyl)benzo[d]imidazo[2,1- b]thiazole-7-carboxamide |
| 116R | 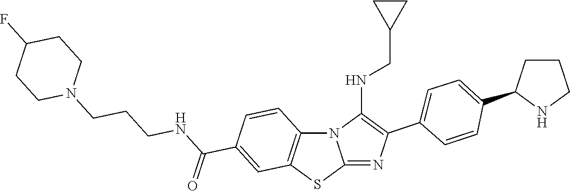 [SMILES: O=C(NCCCN1CCC(F)CC1)c1ccc2c(c1)sc1nc(-c3ccc([C@H]4CCCN4)cc3)c(NCC3CC3)n12] | (R)-3-((cyclopropylmethyl)amino)-N- (3-(4-fluoropiperidin-1-yl)propyl)-2- (4-(pyrrolidin-2- yl)phenyl)benzo[d]imidazo[2,1- b]thiazole-7-carboxamide |
| 117S | 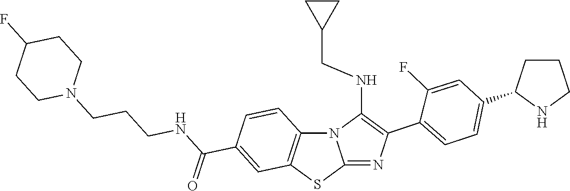 [SMILES: O=C(NCCCN1CCC(F)CC1)c1ccc2c(c1)sc1nc(-c3ccc([C@@H]4CCCN4)cc3F)c(NCC3CC3)n12] | (S)-3-((cyclopropylmethyl)amino)-2- (2-fluoro-4-(pyrrolidin-2-yl)phenyl)- N-(3-(4-fluoropiperidin-1- yl)propyl)benzo[d]imidazo[2,1- b]thiazole-7-carboxamide |
| 117R | 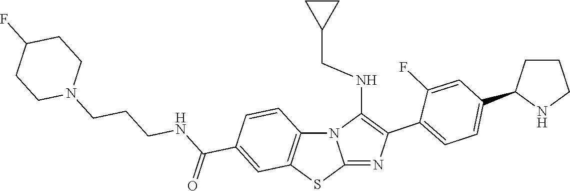 [SMILES: O=C(NCCCN1CCC(F)CC1)c1ccc2c(c1)sc1nc(-c3ccc([C@H]4CCCN4)cc3F)c(NCC3CC3)n12] | (R)-3-((cyclopropylmethyl)amino)-2- (2-fluoro-4-(pyrrolidin-2-yl)phenyl)- N-(3-(4-fluoropiperidin-1- yl)propyl)benzo[d]imidazo[2,1- b]thiazole-7-carboxamide |
| 118S | 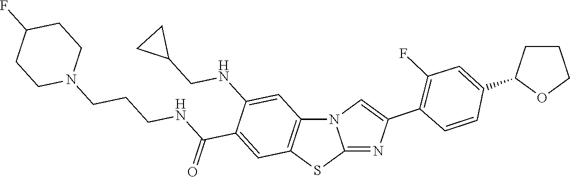 [SMILES: O=C(NCCCN1CCC(F)CC1)c1cc2sc3nc(-c4ccc([C@@H]5CCCO5)cc4F)cn3c2cc1NCC1CC1] | (S)-6-((cyclopropylmethyl)amino)-2- (2-fluoro-4-(tetrahydrofuran-2- yl)phenyl)-N-(3-(4-fluoropiperidin-1- yl)propyl)benzo[d]imidazo[2,1- b]thiazole-7-carboxamide |
| 118R | 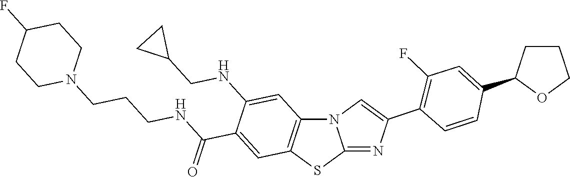 [SMILES: O=C(NCCCN1CCC(F)CC1)c1cc2sc3nc(-c4ccc([C@H]5CCCO5)cc4F)cn3c2cc1NCC1CC1] | (R)-6-((cyclopropylmethyl)amino)-2- (2-fluoro-4-(tetrahydrofuran-2- yl)phenyl)-N-(3-(4-fluoropiperidin-1- yl)propyl)benzo[d]imidazo[2,1- b]thiazole-7-carboxamide |
| 119S | 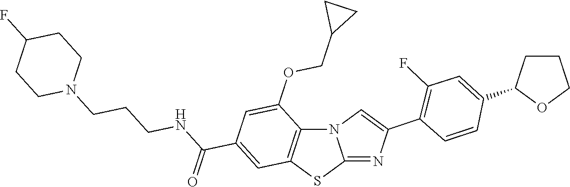 [SMILES: O=C(NCCCN1CCC(F)CC1)c1cc(OCC2CC2)c2c(c1)sc1nc(-c3ccc([C@@H]4CCCO4)cc3F)cn12] | (S)-5-(cyclopropylmethoxy)-2-(2- fluoro-4-(tetrahydrofuran-2- yl)phenyl)-N-(3-(4-fluoropiperidin-1- yl)propyl)benzo[d]imidazo[2,1- b]thiazole-7-carboxamide |
| 119R | 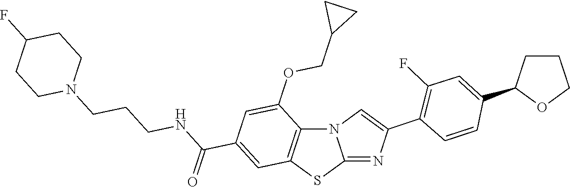 [SMILES: O=C(NCCCN1CCC(F)CC1)c1cc(OCC2CC2)c2c(c1)sc1nc(-c3ccc([C@H]4CCCO4)cc3F)cn12] | (R)-5-(cyclopropylmethoxy)-2-(2- fluoro-4-(tetrahydrofuran-2- yl)phenyl)-N-(3-(4-fluoropiperidin-1- yl)propyl)benzo[d]imidazo[2,1- b]thiazole-7-carboxamide |
| 120S | 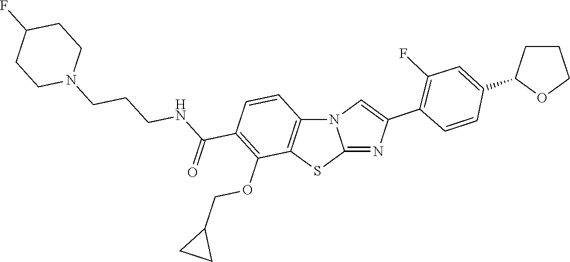 [SMILES: O=C(NCCCN1CCC(F)CC1)c1ccc2c(sc3nc(-c4ccc([C@@H]5CCCO5)cc4F)cn32)c1OCC1CC1] | (S)-8-(cyclopropylmethoxy)-2-(2- fluoro-4-(tetrahydrofuran-2- yl)phenyl)-N-(3-(4-fluoropiperidin-1- yl)propyl)benzo[d]imidazo[2,1- b]thiazole-7-carboxamide |
| 120R | 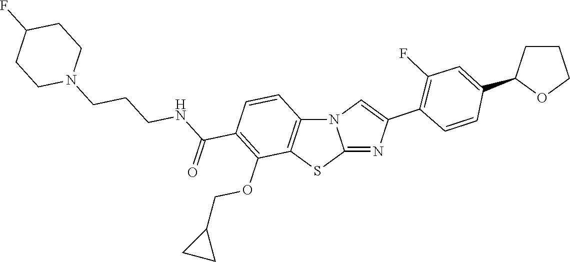 [SMILES: O=C(NCCCN1CCC(F)CC1)c1ccc2c(sc3nc(-c4ccc([C@H]5CCCO5)cc4F)cn32)c1OCC1CC1] | (R)-8-(cyclopropylmethoxy)-2-(2- fluoro-4-(tetrahydrofuran-2- yl)phenyl)-N-(3-(4-fluoropiperidin-1- ·N yl)propyl)benzo[d]imidazo[2,1- b]thiazole-7-carboxamide |
| 121S | 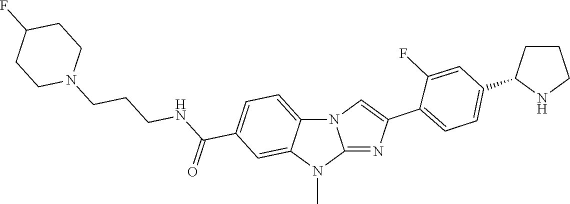 [SMILES: Cn1c2cc(C(=O)NCCCN3CCC(F)CC3)ccc2n2cc(-c3ccc([C@@H]4CCCN4)cc3F)nc12] | (S)-2-(2-fluoro-4-(pyrrolidin-2- yl)phenyl)-N-(3-(4-fluoropiperidin-1- yl)propyl)-9-methyl-9H- benzo[d]imidazo[1,2-a]imidazole-7- carboxamide |
| 121R | 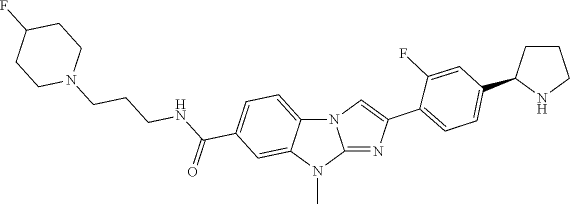 [SMILES: Cn1c2cc(C(=O)NCCCN3CCC(F)CC3)ccc2n2cc(-c3ccc([C@H]4CCCN4)cc3F)nc12] | (R)-2-(2-fluoro-4-(pyrrolidin-2- yl)phenyl)-N-(3-(4-fluoropiperidin-1- yl)propyl)-9-methyl-9H- benzo[d]imidazo[1,2-a]imidazole-7- carboxamide |
| 122S | 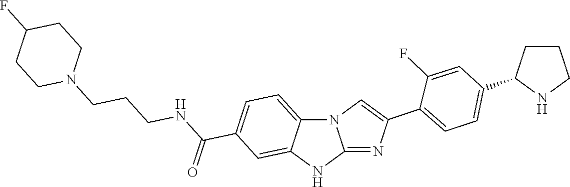 [SMILES: O=C(NCCCN1CCC(F)CC1)c1ccc2c(c1)[nH]c1nc(-c3ccc([C@@H]4CCCN4)cc3F)cn12] | (S)-2-(2-fluoro-4-(pyrrolidin-2- yl)phenyl)-N-(3-(4-fluoropiperidin-1- yl)propyl)-9H-benzo[d]imidazo[1,2- a]imidazole-7-carboxamide |
| 122R | 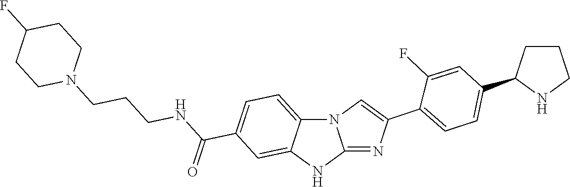 [SMILES: O=C(NCCCN1CCC(F)CC1)c1ccc2c(c1)[nH]c1nc(-c3ccc([C@H]4CCCN4)cc3F)cn12] | (R)-2-(2-fluoro-4-(pyrrolidin-2- yl)phenyl)-N-(3-(4-fluoropiperidin-1- yl)propyl)-9H-benzo[d]imidazo[1,2- a]imidazole-7-carboxamide |
| 123S | 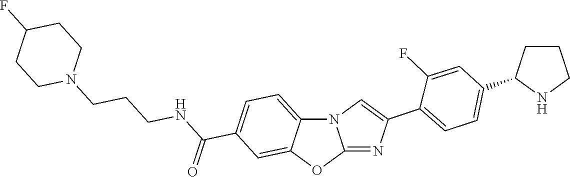 [SMILES: O=C(NCCCN1CCC(F)CC1)c1ccc2c(c1)oc1nc(-c3ccc([C@@H]4CCCN4)cc3F)cn12] | (S)-2-(2-fluoro-4-(pyrrolidin-2- yl)phenyl)-N-(3-(4-fluoropiperidin-1- yl)propyl)benzo[d]imidazo[2,1- b]oxazole-7-carboxamide |
| 123R | 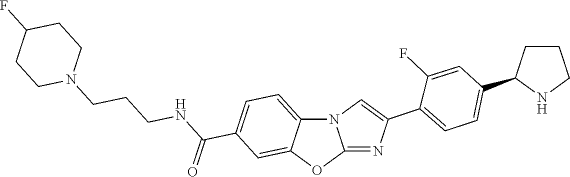 [SMILES: O=C(NCCCN1CCC(F)CC1)c1ccc2c(c1)oc1nc(-c3ccc([C@H]4CCCN4)cc3F)cn12] | (R)-2-(2-fluoro-4-(pyrrolidin-2- yl)phenyl)-N-(3-(4-fluoropiperidin-1- yl)propyl)benzo[d]imidazo[2,1- bloxazole-7-carboxamide |
| 124S | 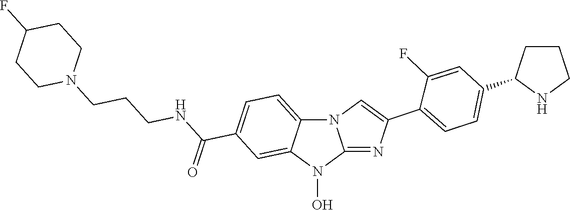 [SMILES: O=C(NCCCN1CCC(F)CC1)c1ccc2c(c1)n(O)c1nc(-c3ccc([C@@H]4CCCN4)cc3F)cn21] | (S)-2-(2-fluoro-4-(pyrrolidin-2- yl)phenyl)-N-(3-(4-fluoropiperidin-1- yl)propyl)-9-hydroxy-9H- benzo[d]imidazo[1,2-a]imidazole-7- carboxamide |
| 124R | 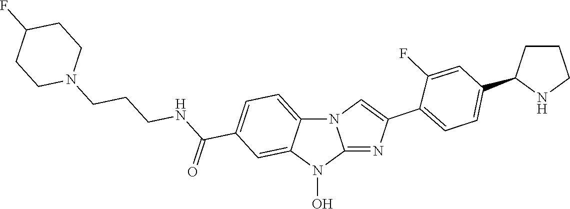 [SMILES: O=C(NCCCN1CCC(F)CC1)c1ccc2c(c1)n(O)c1nc(-c3ccc([C@H]4CCCN4)cc3F)cn21] | (R)-2-(2-fluoro-4-(pyrrolidin-2- yl)phenyl)-N-(3-(4-fluoropiperidin-1- yl)propyl)-9-hydroxy-9H- benzo[d]imidazo[1,2-a]imidazole-7- carboxamide |
| 125S | 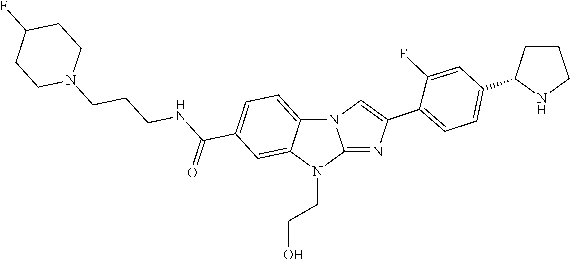 [SMILES: O=C(NCCCN1CCC(F)CC1)c1ccc2c(c1)n(CCO)c1nc(-c3ccc([C@@H]4CCCN4)cc3F)cn21] | (S)-2-(2-fluoro-4-(pyrrolidin-2- yl)phenyl)-N-(3-(4-fluoropiperidin-1- yl)propyl)-9-(2-hydroxyethyl)-9H- benzo[d]imidazo[1,2-a]imidazole-7- carboxamide |
| 125R | 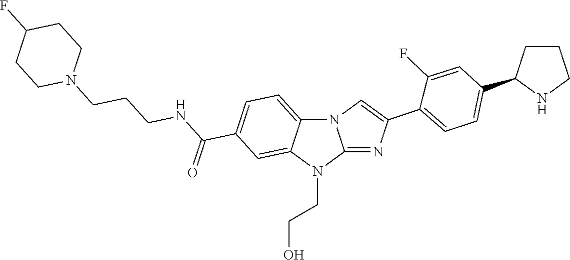 [SMILES: O=C(NCCCN1CCC(F)CC1)c1ccc2c(c1)n(CCO)c1nc(-c3ccc([C@H]4CCCN4)cc3F)cn21] | (R)-2-(2-fluoro-4-(pyrrolidin-2- yl)phenyl)-N-(3-(4-fluoropiperidin-1- yl)propyl)-9-(2-hydroxyethyl)-9H- benzo[d]imidazo[1,2-a]imidazole-7- carboxamide |
| 126S | 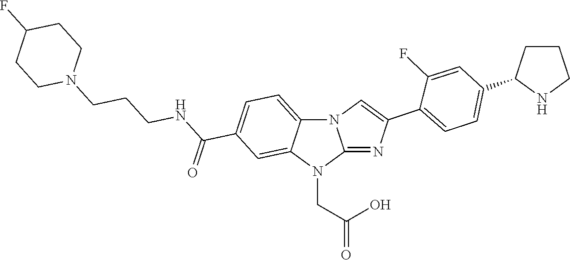 [SMILES: O=C(O)Cn1c2cc(C(=O)NCCCN3CCC(F)CC3)ccc2n2cc(-c3ccc([C@@H]4CCCN4)cc3F)nc12] | (S)-2-(2-(2-fluoro-4-(pyrrolidin-2- yl)phenyl)-7-((3-(4-fluoropiperidin-1- yl)propyl)carbamoyl)-9H- benzo[d]imidazo[1,2-a]imidazol-9- yl)acetic acid |
| 126R | 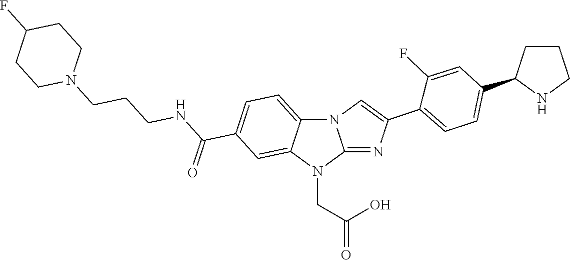 [SMILES: O=C(O)Cn1c2cc(C(=O)NCCCN3CCC(F)CC3)ccc2n2cc(-c3ccc([C@H]4CCCN4)cc3F)nc12] | (R)-2-(2-(2-fluoro-4-(pyrrolidin-2- yl)phenyl)-7-((3-(4-fluoropiperidin-1- yl)propyl)carbamoyl)-9H- benzo[d]imidazo[1,2-a]imidazol-9- yl)acetic acid |
| 127S | 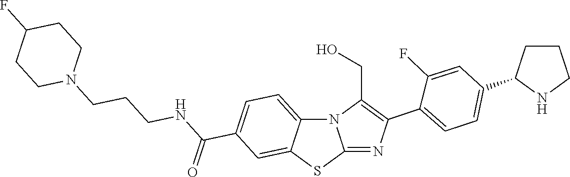 [SMILES: O=C(NCCCN1CCC(F)CC1)c1ccc2c(c1)sc1nc(-c3ccc([C@@H]4CCCN4)cc3F)c(CO)n12] | (S)-2-(2-fluoro-4-(pyrrolidin-2- yl)phenyl)-N-(3-(4-fluoropiperidin-1- yl)propyl)-3- (hydroxymethyl)benzo[d]imidazo [2,1-b]thiazole-7-carboxamide |
| 127R | 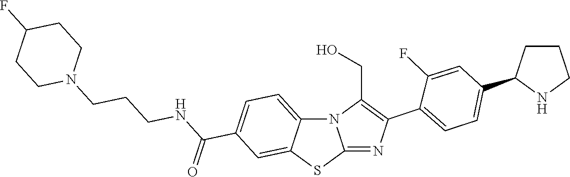 [SMILES: O=C(NCCCN1CCC(F)CC1)c1ccc2c(c1)sc1nc(-c3ccc([C@H]4CCCN4)cc3F)c(CO)n12] | (R)-2-(2-fluoro-4-(pyrrolidin-2- yl)phenyl)-N-(3-(4-fluoropiperidin-1- yl)propyl)-3- (hydroxymethyl)benzo[d]imidazo [2,1-b]thiazole-7-carboxamide |
| 128S | 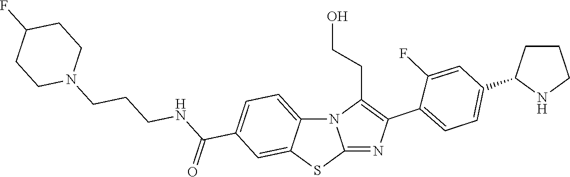 [SMILES: O=C(NCCCN1CCC(F)CC1)c1ccc2c(c1)sc1nc(-c3ccc([C@@H]4CCCN4)cc3F)c(CCO)n12] | (S)-2-(2-fluoro-4-(pyrrolidin-2- yl)phenyl)-N-(3-(4-fluoropiperidin-1- yl)propyl)-3-(2- hydroxyethyl)benzo[d]imidazo[2,1- b]thiazole-7-carboxamide |
| 128R | 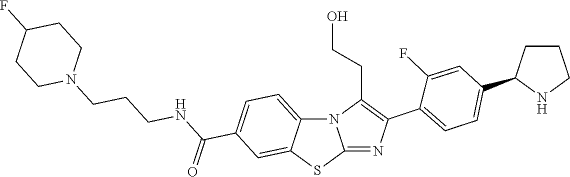 [SMILES: O=C(NCCCN1CCC(F)CC1)c1ccc2c(c1)sc1nc(-c3ccc([C@H]4CCCN4)cc3F)c(CCO)n12] | (R)-2-(2-fluoro-4-(pyrrolidin-2- yl)phenyl)-N-(3-(4-fluoropiperidin-1- yl)propyl)-3-(2- hydroxyethyl)benzo[d]imidazo[2,1- b]thiazole-7-carboxamide |
| 129S | 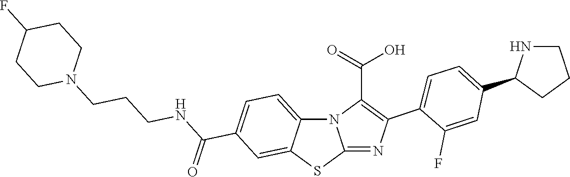 [SMILES: O=C(NCCCN1CCC(F)CC1)c1ccc2c(c1)sc1nc(-c3ccc([C@@H]4CCCN4)cc3F)c(C(=O)O)n12] | (S)-2-(2-fluoro-4-(pyrrolidin-2- yl)phenyl)-7-((3-(4-fluoropiperidin-1- yl)propyl)carbamoyl)benzo[d] imidazo[2,1-b]thiazole- 3-carboxylic acid |
| 129R | 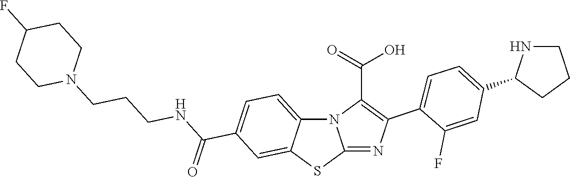 [SMILES: O=C(NCCCN1CCC(F)CC1)c1ccc2c(c1)sc1nc(-c3ccc([C@H]4CCCN4)cc3F)c(C(=O)O)n12] | (R)-2-(2-fluoro-4-(pyrrolidin-2- yl)phenyl)-7-((3-(4-fluoropiperidin-1- yl)propyl)carbamoyl)benzo[d] imidazo[2,1-b]thiazole- 3-carboxylic acid |
| 130R | 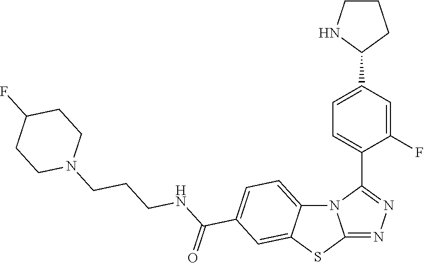 [SMILES: O=C(NCCCN1CCC(F)CC1)c1ccc2c(c1)sc1nnc(-c3ccc([C@H]4CCCN4)cc3F)n12] | (R)-3-(2-fluoro-4-(pyrrolidin-2- yl)phenyl)-N-(3-(4-fluoropiperidin-1- yl)propyl)benzo[4,5]thiazolo[2,3- c][1,2,4]triazole-7-carboxamide |
| 130S | 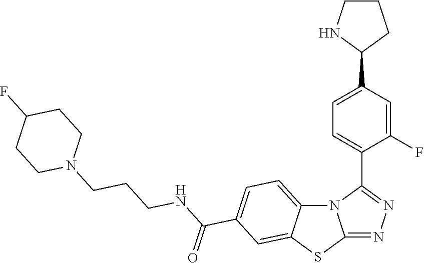 [SMILES: O=C(NCCCN1CCC(F)CC1)c1ccc2c(c1)sc1nnc(-c3ccc([C@@H]4CCCN4)cc3F)n12] | (S)-3-(2-fluoro-4-(pyrrolidin-2- yl)phenyl)-N-(3-(4-fluoropiperidin-1- yl)propyl)benzo[4,5]thiazolo[2,3- c][1,2,4]triazole-7-carboxamide |
| 131S | 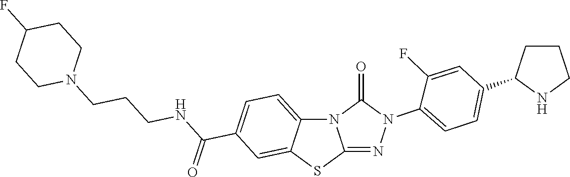 [SMILES: O=C(NCCCN1CCC(F)CC1)c1ccc2c(c1)sc1nn(-c3ccc([C@@H]4CCCN4)cc3F)c(=O)n12] | (S)-2-(2-fluoro-4-(pyrrolidin-2- yl)phenyl)-N-(3-(4-fluoropiperidin-1- yl)propyl)-3-oxo-2,3- dihydrobenzo[4,5]thiazolo[2,3- c][1,2,4]triazole-7-carboxamide |
| 131R | 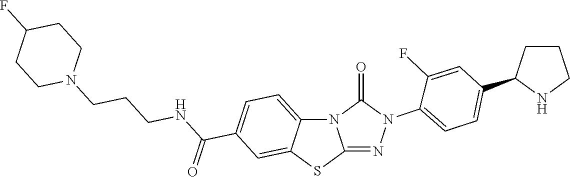 [SMILES: O=C(NCCCN1CCC(F)CC1)c1ccc2c(c1)sc1nn(-c3ccc([C@H]4CCCN4)cc3F)c(=O)n12] | (R)-2-(2-fluoro-4-(pyrrolidin-2- yl)phenyl)-N-(3-(4-fluoropiperidin-1- yl)propyl)-3-oxo-2,3- dihydrobenzo[4,5]thiazolo[2,3- c][1,2,4]triazole-7-carboxamide |
| 132S | 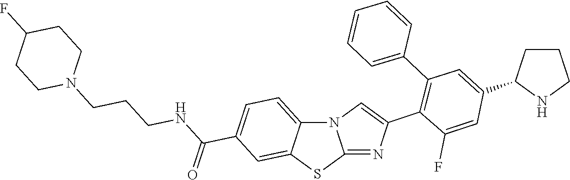 [SMILES: O=C(NCCCN1CCC(F)CC1)c1ccc2c(c1)sc1nc(-c3c(F)cc([C@@H]4CCCN4)cc3-c3ccccc3)cn12] | (S)-2-(3-fluoro-5-(pyrrolidin-2-yl)- [1,1′-biphenyl]-2-yl)-N-(3-(4- fluoropiperidin-1- yl)propyl)benzo[d]imidazo[2,1- b]thiazole-7-carboxamide |
| 132R | 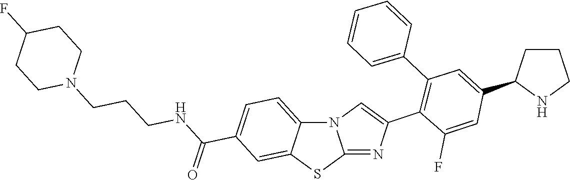 [SMILES: O=C(NCCCN1CCC(F)CC1)c1ccc2c(c1)sc1nc(-c3c(F)cc([C@H]4CCCN4)cc3-c3ccccc3)cn12] | (R)-2-(3-fluoro-5-(pyrrolidin-2-yl)- [1,1′-biphenyl]-2-yl)-N-(3-(4- fluoropiperidin-1- yl)propyl)benzo[d]imidazo[2,1- b]thiazole-7-carboxamide |
| 133S | 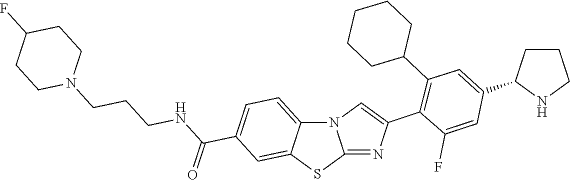 [SMILES: O=C(NCCCN1CCC(F)CC1)c1ccc2c(c1)sc1nc(-c3c(F)cc([C@@H]4CCCN4)cc3C3CCCCC3)cn12] | (S)-2-(2-cyclohexyl-6-fluoro-4- (pyrrolidin-2-yl)phenyl)-N-(3-(4- fluoropiperidin-1- yl)propyl)benzo[d]imidazo[2,1- b]thiazole-7-carboxamide |
| 133R | 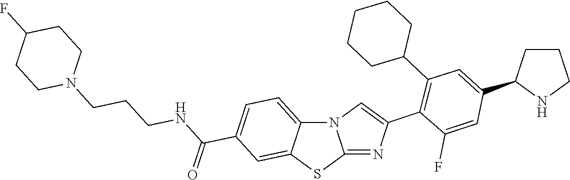 [SMILES: O=C(NCCCN1CCC(F)CC1)c1ccc2c(c1)sc1nc(-c3c(F)cc([C@H]4CCCN4)cc3C3CCCCC3)cn12] | (R)-2-(2-cyclohexyl-6-fluoro-4- (pyrrolidin-2-yl)phenyl)-N-(3-(4- fluoropiperidin-1- yl)propyl)benzo[d]imidazo[2,1- b]thiazole-7-carboxamide |
| 134S | 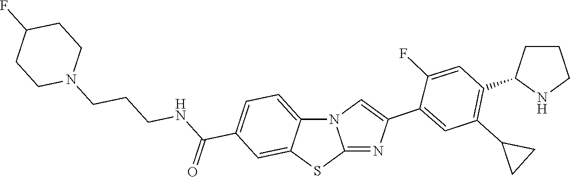 [SMILES: O=C(NCCCN1CCC(F)CC1)c1ccc2c(c1)sc1nc(-c3cc(C4CC4)c([C@@H]4CCCN4)cc3F)cn12] | (S)-2-(5-cyclopropyl-2-fluoro-4- (pyrrolidin-2-yl)phenyl)-N-(3-(4- fluoropiperidin-1- yl)propyl)benzo[d]imidazo[2,1- b]thiazole-7-carboxamide |
| 134R | 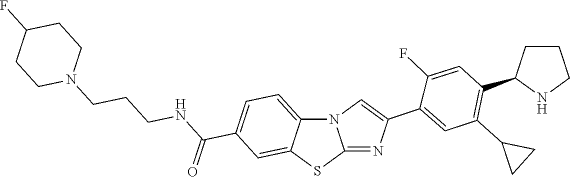 [SMILES: O=C(NCCCN1CCC(F)CC1)c1ccc2c(c1)sc1nc(-c3cc(C4CC4)c([C@H]4CCCN4)cc3F)cn12] | (R)-2-(5-cyclopropyl-2-fluoro-4- (pyrrolidin-2-yl)phenyl)-N-(3-(4- fluoropiperidin-1- yl)propyl)benzo[d]imidazo[2,1- b]thiazole-7-carboxamide |
| 135 | 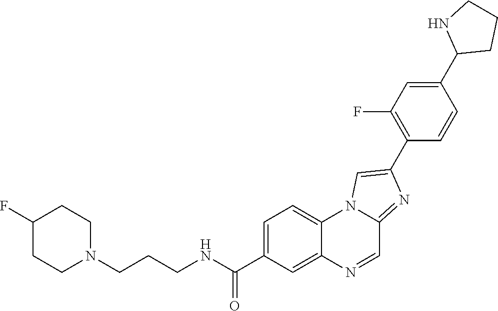 [SMILES: O=C(NCCCN1CCC(F)CC1)c1ccc2c(c1)ncc1nc(-c3ccc(C4CCCN4)cc3F)cn12] | 2-(2-fluoro-4-(pyrrolidin-2- yl)phenyl)-N-(3-(4-fluoropiperidin-1- yl)propyl)imidazo[1,2-a]quinoxaline- 7-carboxamide |
| 136 | 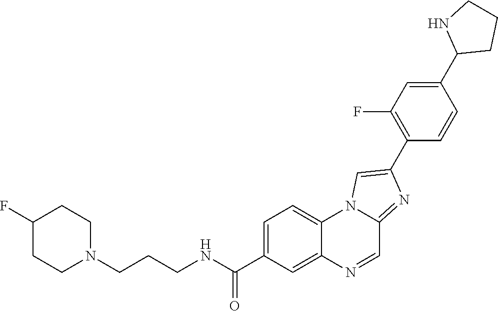 [SMILES: O=C(NCCCN1CCC(F)CC1)c1ccc2c(c1)ncc1nc(-c3ccc(C4CCCN4)cc3F)cn12] | 2-(2-fluoro-4-(pyrrolidin-2- yl)phenyl)-N-(3-(4-fluoropiperidin-1- yl)propyl)imidazo[1,2-a]quinazoline- 7-carboxamide |
| 137S | 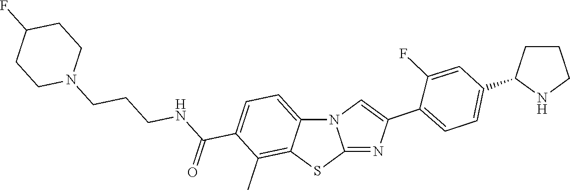 [SMILES: Cc1c(C(=O)NCCCN2CCC(F)CC2)ccc2c1sc1nc(-c3ccc([C@@H]4CCCN4)cc3F)cn12] | (S)-2-(2-fluoro-4-(pyrrolidin-2- yl)phenyl)-N-(3-(4-fluoropiperidin-1- yl)propyl)-8- methylbenzo[d]imidazo[2,1- b]thiazole-7-carboxamide |
| 137R | 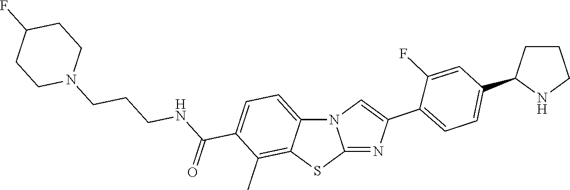 [SMILES: Cc1c(C(=O)NCCCN2CCC(F)CC2)ccc2c1sc1nc(-c3ccc([C@H]4CCCN4)cc3F)cn12] | (R)-2-(2-fluoro-4-(pyrrolidin-2- yl)phenyl)-N-(3-(4-fluoropiperidin-1- yl)propyl)-8- methylbenzo[d]imidazo[2,1- b]thiazole-7-carboxamide |
| 138 | 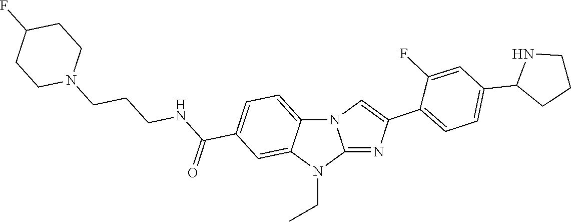 [SMILES: CCn1c2cc(C(=O)NCCCN3CCC(F)CC3)ccc2n2cc(-c3ccc(C4CCCN4)cc3F)nc12] | 9-ethyl-2-(2-fluoro-4-(pyrrolidin-2- yl)phenyl)-N-(3-(4-fluoropiperidin-1- yl)propyl)-9H-benzo[d]imidazo[1,2- a]imidazole-7-carboxamide |
| 139 | 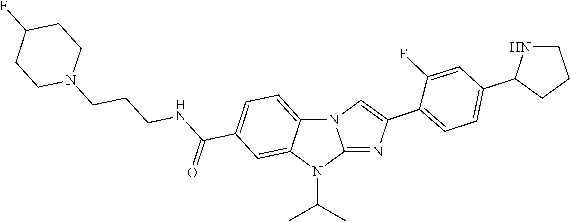 [SMILES: CC(C)n1c2cc(C(=O)NCCCN3CCC(F)CC3)ccc2n2cc(-c3ccc(C4CCCN4)cc3F)nc12] | 2-(2-fluoro-4-(pyrrolidin-2- yl)phenyl)-N-(3-(4-fluoropiperidin-1- yl)propyl)-9-isopropyl-9H- benzo[d]imidazo[1,2-a]imidazole-7- carboxamide |
| 140 | 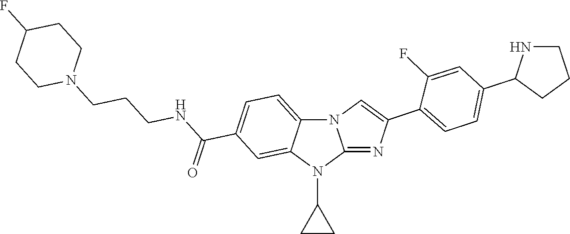 [SMILES: O=C(NCCCN1CCC(F)CC1)c1ccc2c(c1)n(C1CC1)c1nc(-c3ccc(C4CCCN4)cc3F)cn21] | 9-cyclopropyl-2-(2-fluoro-4- (pyrrolidin-2-yl)phenyl)-N-(3-(4- fluoropiperidin-1-yl)propyl)-9H- benzo[d]imidazo[1,2-a]imidazole-7- carboxamide |
| 141 | 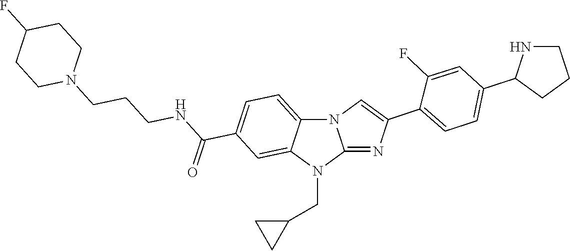 [SMILES: O=C(NCCCN1CCC(F)CC1)c1ccc2c(c1)n(CC1CC1)c1nc(-c3ccc(C4CCCN4)cc3F)cn21] | 9-(cyclopropylmethyl)-2-(2-fluoro-4- (pyrrolidin-2-yl)phenyl)-N-(3-(4- fluoropiperidin-1-yl)propyl)-9H- benzo[d]imidazo[1,2-a]imidazole-7- carboxamide |
| 142 | 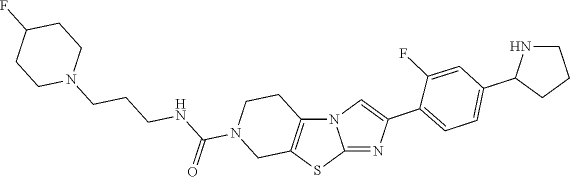 [SMILES: O=C(NCCCN1CCC(F)CC1)N1CCc2c(sc3nc(-c4ccc(C5CCCN5)cc4F)cn23)C1] | 2-(2-fluoro-4-(pyrrolidin-2- yl)phenyl)-N-(3-(4-fluoropiperidin-1- yl)propyl)-5,8- dihydroimidazo[2′,1′:2,3]thiazolo [5,4-c]pyridine-7(6H)-carboxamide |
| 143 | 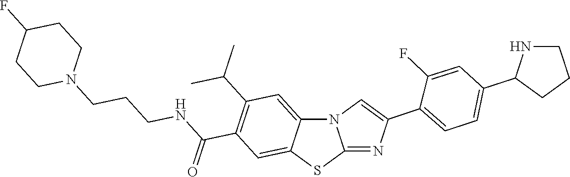 [SMILES: CC(C)c1cc2c(cc1C(=O)NCCCN1CCC(F)CC1)sc1nc(-c3ccc(C4CCCN4)cc3F)cn12] | 2-(2-fluoro-4-(pyrrolidin-2- yl)phenyl)-N-(3-(4-fluoropiperidin-1- yl)propyl)-6- isopropylbenzo[d]imidazo[2,1- b]thiazole-7-carboxamide |
| 144 | 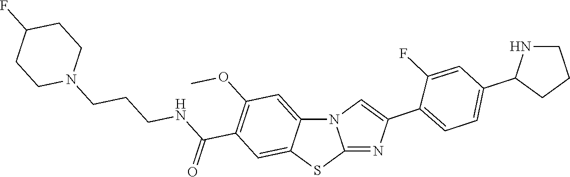 [SMILES: COc1cc2c(cc1C(=O)NCCCN1CCC(F)CC1)sc1nc(-c3ccc(C4CCCN4)cc3F)cn12] | 2-(2-fluoro-4-(pyrrolidin-2- yl)phenyl)-N-(3-(4-fluoropiperidin-1- yl)propyl)-6- methoxybenzo[d]imidazo[2,1- b]thiazole-7-carboxamide |
| 145 | 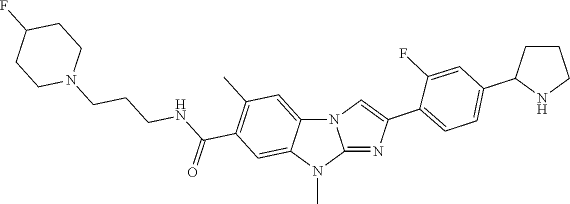 [SMILES: Cc1cc2c(cc1C(=O)NCCCN1CCC(F)CC1)n(C)c1nc(-c3ccc(C4CCCN4)cc3F)cn21] | 2-(2-fluoro-4-(pyrrolidin-2- yl)phenyl)-N-(3-(4-fluoropiperidin-1- yl)propyl)-6,9-dimethyl-9H- benzo[d]imidazo[1,2-a]imidazole-7- carboxamide |
| 146 | 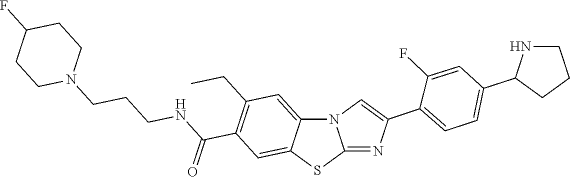 [SMILES: CCc1cc2c(cc1C(=O)NCCCN1CCC(F)CC1)sc1nc(-c3ccc(C4CCCN4)cc3F)cn12] | 6-ethyl-2-(2-fluoro-4-(pyrrolidin-2- yl)phenyl)-N-(3-(4-fluoropiperidin-1- yl)propyl)benzo[d]imidazo[2,1- b]thiazole-7-carboxamide |
| 147 | 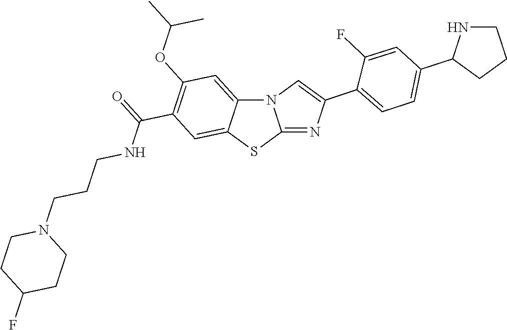 [SMILES: CC(C)Oc1cc2c(cc1C(=O)NCCCN1CCC(F)CC1)sc1nc(-c3ccc(C4CCCN4)cc3F)cn12] | 2-(2-fluoro-4-(pyrrolidin-2- yl)phenyl)-N-(3-(4-fluoropiperidin-1- yl)propyl)-6- isopropoxybenzo[d]imidazo[2,1- b]thiazole-7-carboxamide |
| 148 | 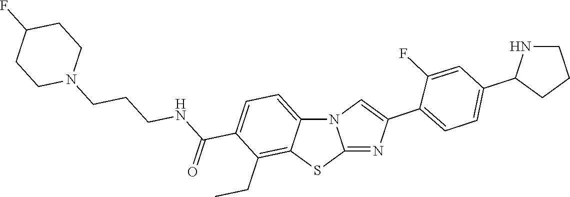 [SMILES: CCc1c(C(=O)NCCCN2CCC(F)CC2)ccc2c1sc1nc(-c3ccc(C4CCCN4)cc3F)cn12] | 8-ethyl-2-(2-fluoro-4-(pyrrolidin-2- yl)phenyl)-N-(3-(4-fluoropiperidin-1- yl)propyl)benzo[d]imidazo[2,1- b]thiazole-7-carboxamide |
| 149S | 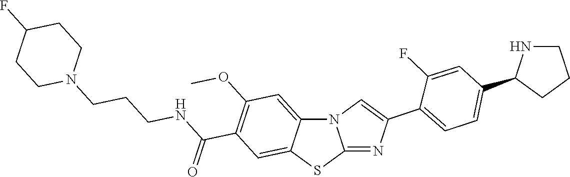 [SMILES: COc1cc2c(cc1C(=O)NCCCN1CCC(F)CC1)sc1nc(-c3ccc([C@@H]4CCCN4)cc3F)cn12] | (S)-2-(2-fluoro-4-(pyrrolidin-2- yl)phenyl)-N-(3-(4-fluoropiperidin-1- yl)propyl)-6- methoxybenzo[d]imidazo[2,1- b]thiazole-7-carboxamide |
| 149R | 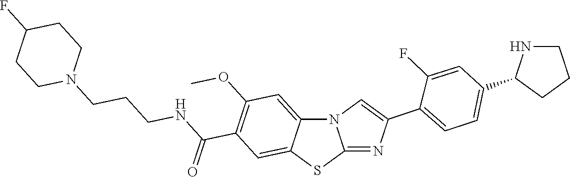 [SMILES: COc1cc2c(cc1C(=O)NCCCN1CCC(F)CC1)sc1nc(-c3ccc([C@H]4CCCN4)cc3F)cn12] | (R)-2-(2-fluoro-4-(pyrrolidin-2- yl)phenyl)-N-(3-(4-fluoropiperidin-1- yl)propyl)-6- methoxybenzo[d]imidazo[2,1- b]thiazole-7-carboxamide |
| 150 | 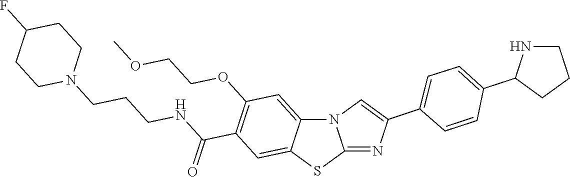 [SMILES: COCCOc1cc2c(cc1C(=O)NCCCN1CCC(F)CC1)sc1nc(-c3ccc(C4CCCN4)cc3)cn12] | N-(3-(4-fluoropiperidin-1-yl)propyl)- 6-(2-methoxyethoxy)-2-(4- (pyrrolidin-2- yl)phenyl)benzo[d]imidazo[2,1- b]thiazole-7-carboxamide |
| 151 | 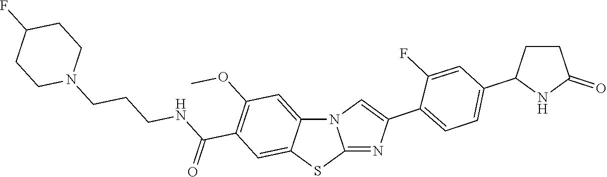 [SMILES: COc1cc2c(cc1C(=O)NCCCN1CCC(F)CC1)sc1nc(-c3ccc(C4CCC(=O)N4)cc3F)cn12] | 2-(2-fluoro-4-(5-oxopyrrolidin-2- yl)phenyl)-N-(3-(4-fluoropiperidin-1- yl)propyl)-6- methoxybenzo[d]imidazo[2,1- b]thiazole-7-carboxamide |
| 152 | 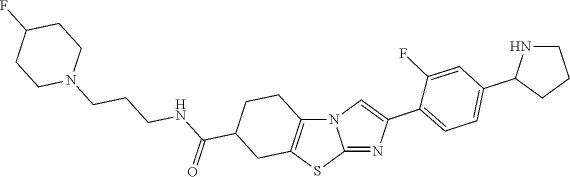 [SMILES: O=C(NCCCN1CCC(F)CC1)C1CCc2c(sc3nc(-c4ccc(C5CCCN5)cc4F)cn23)C1] | 2-(2-fluoro-4-(pyrrolidin-2- yl)phenyl)-N-(3-(4-fluoropiperidin-1- yl)propyl)-5,6,7,8- tetrahydrobenzo[d]imidazo[2,1- b]thiazole-7-carboxamide |
| 153S | 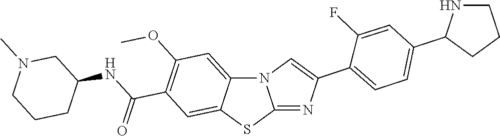 [SMILES: COc1cc2c(cc1C(=O)N[C@H]1CCCN(C)C1)sc1nc(-c3ccc(C4CCCN4)cc3F)cn12] | 2-(2-fluoro-4-(pyrrolidin-2- yl)phenyl)-6-methoxy-N-((S)-1- methylpiperidin-3- yl)benzo[d]imidazo[2,1-b]thiazole-7- carboxamide |
| 153R | 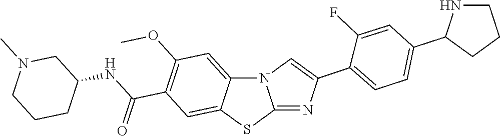 [SMILES: COc1cc2c(cc1C(=O)N[C@@H]1CCCN(C)C1)sc1nc(-c3ccc(C4CCCN4)cc3F)cn12] | 2-(2-fluoro-4-(pyrrolidin-2- yl)phenyl)-6-methoxy-N-((R)-1- methylpiperidin-3- yl)benzo[d]imidazo[2,1-b]thiazole-7- carboxamide |
| 154 | 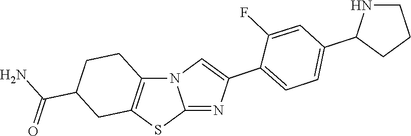 [SMILES: NC(=O)C1CCc2c(sc3nc(-c4ccc(C5CCCN5)cc4F)cn23)C1] | 2-(2-fluoro-4-(pyrrolidin-2- yl)phenyl)-5,6,7,8- tetrahydrobenzo[d]imidazo[2,1- b]thiazole-7-carboxamide |
| 155 | 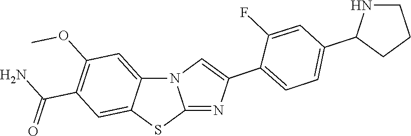 [SMILES: COc1cc2c(cc1C(N)=O)sc1nc(-c3ccc(C4CCCN4)cc3F)cn12] | 2-(2-fluoro-4-(pyrrolidin-2- yl)phenyl)-6- methoxybenzo[d]imidazo[2,1- b]thiazole-7-carboxamide |
| 156 | 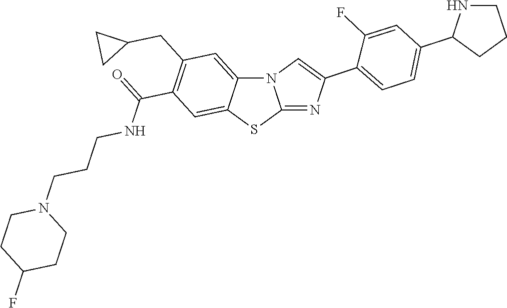 [SMILES: O=C(NCCCN1CCC(F)CC1)c1cc2sc3nc(-c4ccc(C5CCCN5)cc4F)cn3c2cc1CC1CC1] | 6-(cyclopropylmethyl)-2-(2-fluoro-4- (pyrrolidin-2-yl)phenyl)-N-(3-(4- fluoropiperidin-1- yl)propyl)benzo[d]imidazo[2,1- b]thiazole-7-carboxamide |
| 157 | 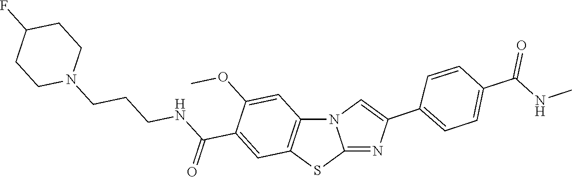 [SMILES: CNC(=O)c1ccc(-c2cn3c(n2)sc2cc(C(=O)NCCCN4CCC(F)CC4)c(OC)cc23)cc1] | N-(3-(4-fluoropiperidin-1-yl)propyl)- 6-methoxy-2-(4- (methylcarbamoyl)phenyl)benzo[d]i midazo[2,1-b]thiazole-7- carboxamide |
| 158 | 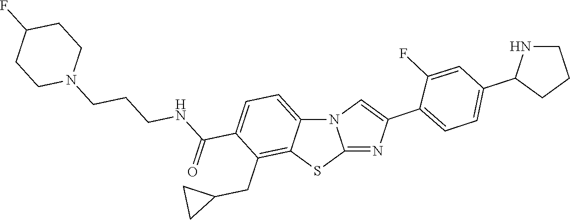 [SMILES: O=C(NCCCN1CCC(F)CC1)c1ccc2c(sc3nc(-c4ccc(C5CCCN5)cc4F)cn32)c1CC1CC1] | 8-(cyclopropylmethyl)-2-(2-fluoro-4- (pyrrolidin-2-yl)phenyl)-N-(3-(4- fluoropiperidin-1- yl)propyl)benzo[d]imidazo[2,1- b]thiazole-7-carboxamide |
| 159 | 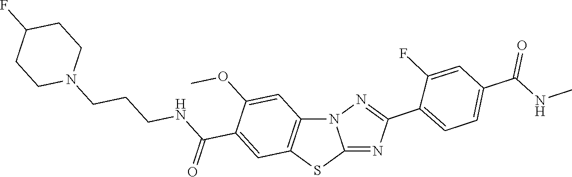 [SMILES: CNC(=O)c1ccc(-c2nc3sc4cc(C(=O)NCCCN5CCC(F)CC5)c(OC)cc4n3n2)c(F)c1] | 2-(2-fluoro-4- (methylcarbamoyl)phenyl)-N-(3-(4- fluoropiperidin-1-yl)propyl)-6- methoxybenzo[d]imidazo[2,1- b]thiazole-7-carboxamide |
| 160 | 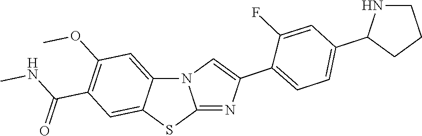 [SMILES: CNC(=O)c1cc2sc3nc(-c4ccc(C5CCCN5)cc4F)cn3c2cc1OC] | 2-(2-fluoro-4-(pyrrolidin-2- yl)phenyl)-6-methoxy-N- methylbenzo[d]imidazo[2,1- b]thiazole-7-carboxamide |
| 161 | 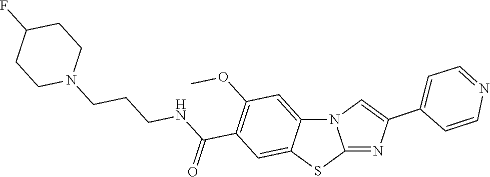 [SMILES: COc1cc2c(cc1C(=O)NCCCN1CCC(F)CC1)sc1nc(-c3ccncc3)cn12] | N-(3-(4-fluoropiperidin-1-yl)propyl)- 6-methoxy-2-(pyridin-4- yl)benzo[d]imidazo[2,1-b]thiazole-7- carboxamide |
| 162 | 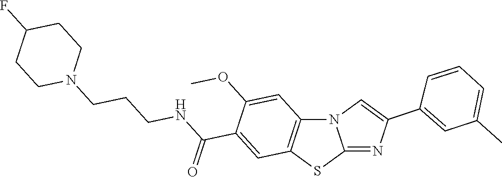 [SMILES: COc1cc2c(cc1C(=O)NCCCN1CCC(F)CC1)sc1nc(-c3cccc(C)c3)cn12] | N-(3-(4-fluoropiperidin-1-yl)propyl)- 6-methoxy-2-(m- tolyl)benzo[d]imidazo[2,1- b]thiazole-7-carboxamide |
| 163 | 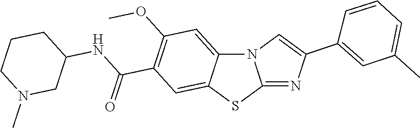 [SMILES: COc1cc2c(cc1C(=O)NC1CCCN(C)C1)sc1nc(-c3cccc(C)c3)cn12] | 6-methoxy-N-(1-methylpiperidin-3- yl)-2-(m-tolyl)benzo[d]imidazo[2,1- b]thiazole-7-carboxamide |
| 164 | 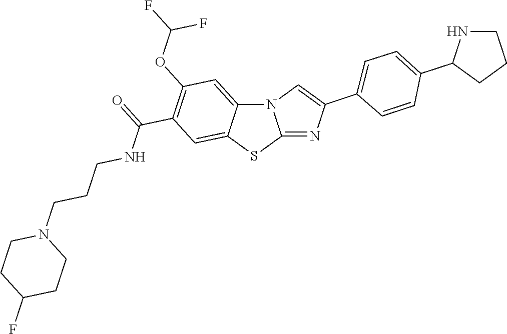 [SMILES: O=C(NCCCN1CCC(F)CC1)c1cc2sc3nc(-c4ccc(C5CCCN5)cc4)cn3c2cc1OC(F)F] | 6-(difluoromethoxy)-N-(3-(4- fluoropiperidin-1-yl)propyl)-2-(4- (pyrrolidin-2- yl)phenyl)benzo[d]imidazo[2,1- b]thiazole-7-carboxamide |
| 165 | 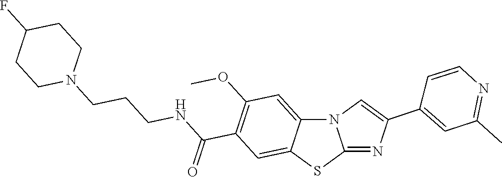 [SMILES: COc1cc2c(cc1C(=O)NCCCN1CCC(F)CC1)sc1nc(-c3ccnc(C)c3)cn12] | N-(3-(4-fluoropiperidin-1-yl)propyl)- 6-methoxy-2-(2-methylpyridin-4- yl)benzo[d]imidazo[2,1-b]thiazole-7- carboxamide |
| 166 | 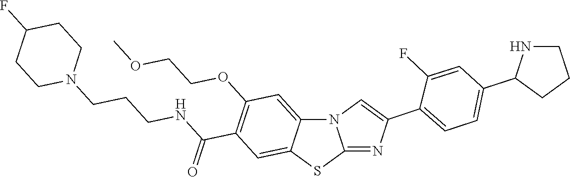 [SMILES: COCCOc1cc2c(cc1C(=O)NCCCN1CCC(F)CC1)sc1nc(-c3ccc(C4CCCN4)cc3F)cn12] | 2-(2-fluoro-4-(pyrrolidin-2- yl)phenyl)-N-(3-(4-fluoropiperidin-1- yl)propyl)-6-(2- methoxyethoxy)benzo[d]imidazo[2,1- b]thiazole-7-carboxamide |
| 167 | 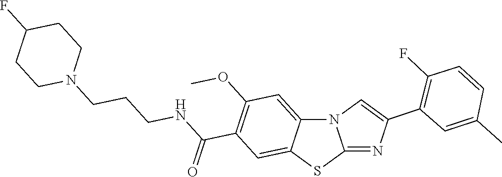 [SMILES: COc1cc2c(cc1C(=O)NCCCN1CCC(F)CC1)sc1nc(-c3cc(C)ccc3F)cn12] | 2-(2-fluoro-5-methylphenyl)-N-(3-(4- fluoropiperidin-1-yl)propyl)-6- methoxybenzo[d]imidazo[2,1- b]thiazole-7-carboxamide |
| 168 | 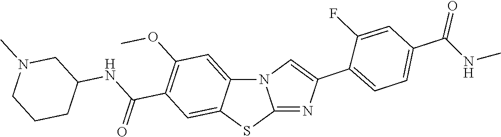 | 2-(2-fluoro-4- (methylcarbamoyl)phenyl)-6- methoxy-N-(1-methylpiperidin-3- yl)benzo[d]imidazo[2,1-b]thiazole-7- carboxamide |
| 169 | 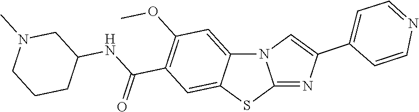 [SMILES: COc1cc2c(cc1C(=O)NC1CCCN(C)C1)sc1nc(-c3ccncc3)cn12] | 6-methoxy-N-(1-methylpiperidin-3- yl)-2-(pyridin-4- yl)benzo[d]imidazo[2,1-b]thiazole-7- carboxamide |
| 170 | 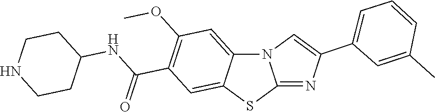 [SMILES: COc1cc2c(cc1C(=O)NC1CCNCC1)sc1nc(-c3cccc(C)c3)cn12] | 6-methoxy-N-(piperidin-4-yl)-2-(m- tolyl)benzo[d]imidazo[2,1- b]thiazole-7-carboxamide |
| 171 | 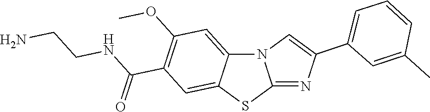 [SMILES: COc1cc2c(cc1C(=O)NCCN)sc1nc(-c3cccc(C)c3)cn12] | N-(2-aminoethyl)-6-methoxy-2-(m- tolyl)benzo[d]imidazo[2,1- b]thiazole-7-carboxamide |
| 172 | 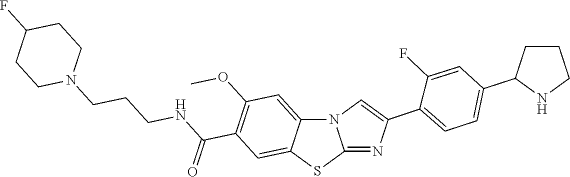 [SMILES: COc1cc2c(cc1C(=O)NCCCN1CCC(F)CC1)sc1nc(-c3ccc(C4CCCN4)cc3F)cn12] | 2-(2-fluoro-4-(pyrrolidin-2- yl)phenyl)-N-(3-(4-fluoropiperidin-1- yl)propyl)-6- methoxybenzo[d]imidazo[2,1- b]oxazole-7-carboxamide |
| 173 | 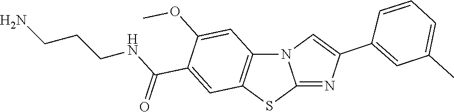 [SMILES: COc1cc2c(cc1C(=O)NCCCN)sc1nc(-c3cccc(C)c3)cn12] | N-(3-aminopropyl)-6-methoxy-2-(m- tolyl)benzo[d]imidazo[2,1- b]thiazole-7-carboxamide |
| 174 | 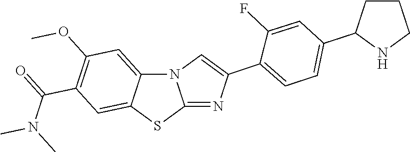 [SMILES: COc1cc2c(cc1C(=O)N(C)C)sc1nc(-c3ccc(C4CCCN4)cc3F)cn12] | 2-(2-fluoro-4-(pyrrolidin-2- yl)phenyl)-6-methoxy-N,N- dimethylbenzo[d]imidazo[2,1- b]thiazole-7-carboxamide |
| 175 | 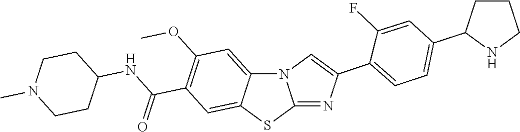 [SMILES: COc1cc2c(cc1C(=O)NC1CCN(C)CC1)sc1nc(-c3ccc(C4CCCN4)cc3F)cn12] | 2-(2-fluoro-4-(pyrrolidin-2- yl)phenyl)-6-methoxy-N-(1- methylpiperidin-4- yl)benzo[d]imidazo[2,1-b]thiazole-7- carboxamide |
| 176 | 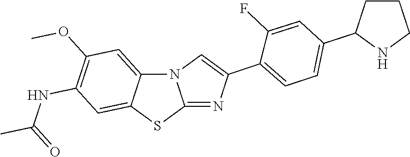 [SMILES: COc1cc2c(cc1NC(C)=O)sc1nc(-c3ccc(C4CCCN4)cc3F)cn12] | N-(2-(2-fluoro-4-(pyrrolidin-2- yl)phenyl)-6- methoxybenzo[d]imidazo[2,1- b]thiazol-7-yl)acetamide |
| 177 | 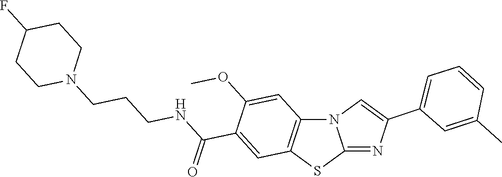 [SMILES: COc1cc2c(cc1C(=O)NCCCN1CCC(F)CC1)sc1nc(-c3cccc(C)c3)cn12] | N-(3-(4-fluoropiperidin-1-yl)propyl)- 7-methoxy-2-(m- tolyl)benzo[4,5]thiazolo[3,2- b][1,2,4]triazole-6-carboxamide |
| 178 | 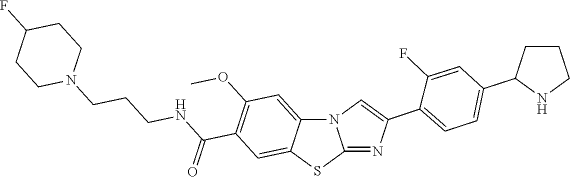 [SMILES: COc1cc2c(cc1C(=O)NCCCN1CCC(F)CC1)sc1nc(-c3ccc(C4CCCN4)cc3F)cn12] | 2-(2-fluoro-4-(pyrrolidin-2- yl)phenyl)-N-(3-(4-fluoropiperidin-1- yl)propyl)-7- methoxybenzo[4,5]thiazolo[3,2- b][1,2,4]triazole-6-carboxamide |
| 179 | 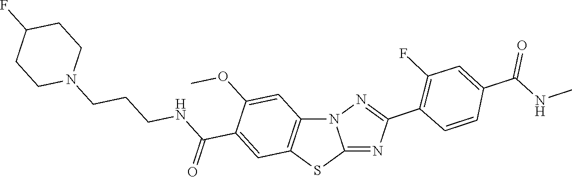 [SMILES: CNC(=O)c1ccc(-c2nc3sc4cc(C(=O)NCCCN5CCC(F)CC5)c(OC)cc4n3n2)c(F)c1] | 2-(2-fluoro-4- (methylcarbamoyl)phenyl)-N-(3-(4- fluoropiperidin-1-yl)propyl)-7- methoxybenzo[4,5]thiazolo[3,2- b][1,2,4]triazole-6-carboxamide |
| 180 | 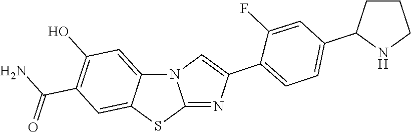 [SMILES: NC(=O)c1cc2sc3nc(-c4ccc(C5CCCN5)cc4F)cn3c2cc1O] | 2-(2-fluoro-4-(pyrrolidin-2- yl)phenyl)-6- hydroxybenzo[d]imidazo[2,1- b]thiazole-7-carboxamide |
| 181 | 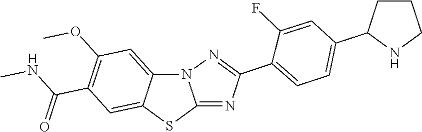 [SMILES: CNC(=O)c1cc2sc3nc(-c4ccc(C5CCCN5)cc4F)nn3c2cc1OC] | 2-(2-fluoro-4-(pyrrolidin-2- yl)phenyl)-7-methoxy-N- methylbenzo[4,5]thiazolo[3,2- b][1,2,4]triazole-6-carboxamide |
| 182 | 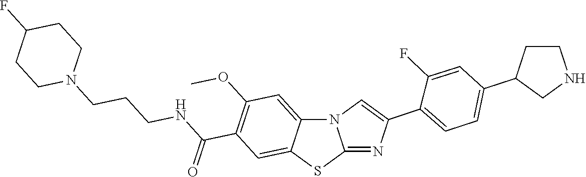 [SMILES: COc1cc2c(cc1C(=O)NCCCN1CCC(F)CC1)sc1nc(-c3ccc(C4CCNC4)cc3F)cn12] | 2-(2-fluoro-4-(pyrrolidin-3- yl)phenyl)-N-(3-(4-fluoropiperidin-1- yl)propyl)-6- methoxybenzo[d]imidazo[2,1- b]thiazole-7-carboxamide |
| 183 | 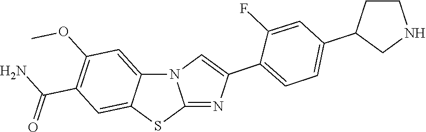 [SMILES: COc1cc2c(cc1C(N)=O)sc1nc(-c3ccc(C4CCNC4)cc3F)cn12] | 2-(2-fluoro-4-(pyrrolidin-3- yl)phenyl)-6- methoxybenzo[d]imidazo[2,1- b]thiazole-7-carboxamide |
| 184 | 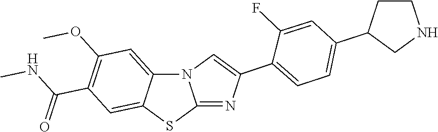 [SMILES: CNC(=O)c1cc2sc3nc(-c4ccc(C5CCNC5)cc4F)cn3c2cc1OC] | 2-(2-fluoro-4-(pyrrolidin-3- yl)phenyl)-6-methoxy-N- methylbenzo[d]imidazo[2,1- b]thiazole-7-carboxamide |
| 185 | 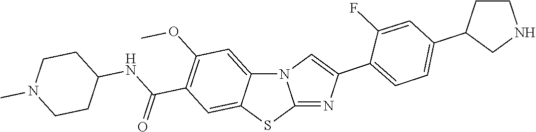 [SMILES: COc1cc2c(cc1C(=O)NC1CCN(C)CC1)sc1nc(-c3ccc(C4CCNC4)cc3F)cn12] | 2-(2-fluoro-4-(pyrrolidin-3- yl)phenyl)-6-methoxy-N-(1- methylpiperidin-4- yl)benzo[d]imidazo[2,1-b]thiazole-7- carboxamide |
| 186 | 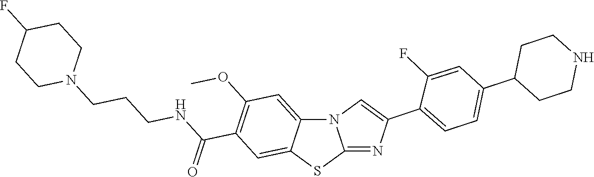 [SMILES: COc1cc2c(cc1C(=O)NCCCN1CCC(F)CC1)sc1nc(-c3ccc(C4CCNCC4)cc3F)cn12] | 2-(2-fluoro-4-(piperidin-4-yl)phenyl)- N-(3-(4-fluoropiperidin-1-yl)propyl)- 6-methoxybenzo[d]imidazo[2,1- b]thiazole-7-carboxamide |
| 187 | 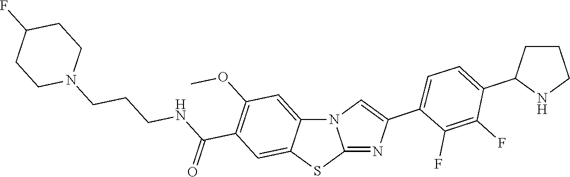 [SMILES: COc1cc2c(cc1C(=O)NCCCN1CCC(F)CC1)sc1nc(-c3ccc(C4CCCN4)c(F)c3F)cn12] | 2-(2,3-difluoro-4-(pyrrolidin-2- yl)phenyl)-N-(3-(4-fluoropiperidin-1- yl)propyl)-6- methoxybenzo[d]imidazo[2,1- b]thiazole-7-carboxamide |
| 188 | 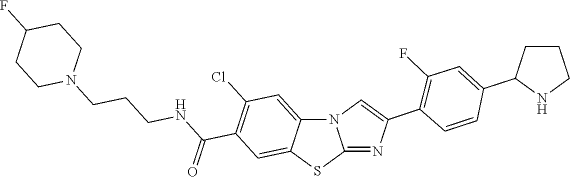 [SMILES: O=C(NCCCN1CCC(F)CC1)c1cc2sc3nc(-c4ccc(C5CCCN5)cc4F)cn3c2cc1Cl] | 6-chloro-2-(2-fluoro-4-(pyrrolidin-2- yl)phenyl)-N-(3-(4-fluoropiperidin-1- yl)propyl)benzo[d]imidazo[2,1- b]thiazole-7-carboxamide |
| 189 | 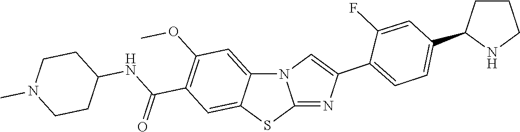 [SMILES: COc1cc2c(cc1C(=O)NC1CCN(C)CC1)sc1nc(-c3ccc([C@H]4CCCN4)cc3F)cn12] | (R)-2-(2-fluoro-4-(pyrrolidin-2- yl)phenyl)-6-methoxy-N-(1- methylpiperidin-4- yl)benzo[d]imidazo[2,1-b]thiazole-7- carboxamide |
| 190 | 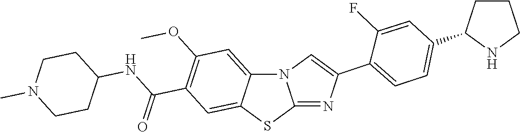 [SMILES: COc1cc2c(cc1C(=O)NC1CCN(C)CC1)sc1nc(-c3ccc([C@@H]4CCCN4)cc3F)cn12] | (S)-2-(2-fluoro-4-(pyrrolidin-2- yl)phenyl)-6-methoxy-N-(1- methylpiperidin-4- yl)benzo[d]imidazo[2,1-b]thiazole-7- carboxamide |
| 191 | 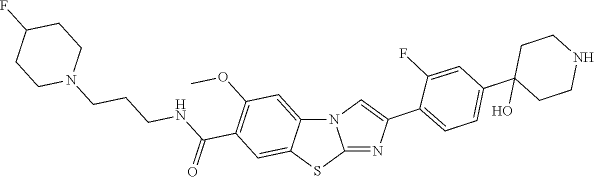 [SMILES: COc1cc2c(cc1C(=O)NCCCN1CCC(F)CC1)sc1nc(-c3ccc(C4(O)CCNCC4)cc3F)cn12] | 2-(2-fluoro-4-(4-hydroxypiperidin-4- yl)phenyl)-N-(3-(4-fluoropiperidin-1- yl)propyl)-6- methoxybenzo[d]imidazo[2,1- b]thiazole-7-carboxamide |
| 192 | 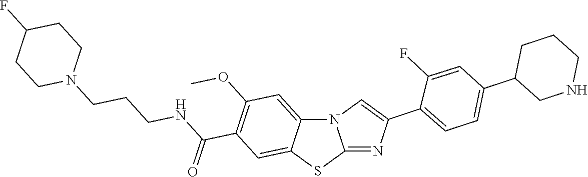 [SMILES: COc1cc2c(cc1C(=O)NCCCN1CCC(F)CC1)sc1nc(-c3ccc(C4CCCNC4)cc3F)cn12] | 2-(2-fluoro-4-(piperidin-3-yl)phenyl)- N-(3-(4-fluoropiperidin-1-yl)propyl)- 6-methoxybenzo[d]imidazo[2,1- b]thiazole-7-carboxamide |
| 195 | 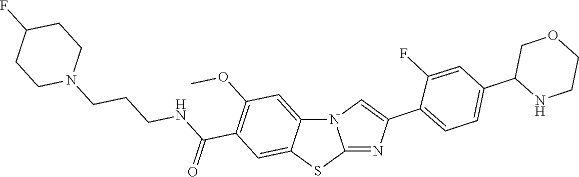 [SMILES: COc1cc2c(cc1C(=O)NCCCN1CCC(F)CC1)sc1nc(-c3ccc(C4COCCN4)cc3F)cn12] | 2-(2-fluoro-4-(morpholin-3- yl)phenyl)-N-(3-(4-fluoropiperidin-1- yl)propyl)-6- methoxybenzo[d]imidazo[2,1- b]thiazole-7-carboxamide |
| 196 | 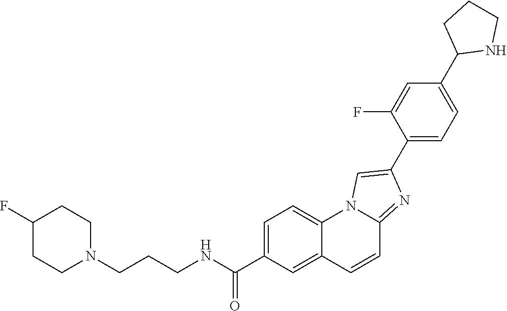 [SMILES: O=C(NCCCN1CCC(F)CC1)c1ccc2c(ccc3nc(-c4ccc(C5CCCN5)cc4F)cn32)c1] | 2-(2-fluoro-4-(pyrrolidin-2- yl)phenyl)-N-(3-(4-fluoropiperidin-1- yl)propyl)imidazo[1,2-a]quinoline-7- carboxamide |
| 197 | 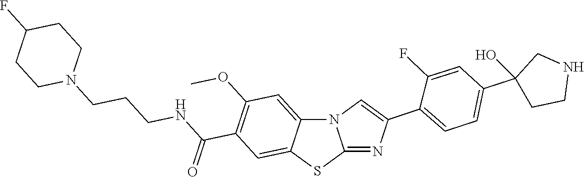 [SMILES: COc1cc2c(cc1C(=O)NCCCN1CCC(F)CC1)sc1nc(-c3ccc(C4(O)CCNC4)cc3F)cn12] | 2-(2-fluoro-4-(3-hydroxypyrrolidin- 3-yl)phenyl)-N-(3-(4-fluoropiperidin- 1-yl)propyl)-6- methoxybenzo[d]imidazo[2,1- b]thiazole-7-carboxamide |
| 198 | 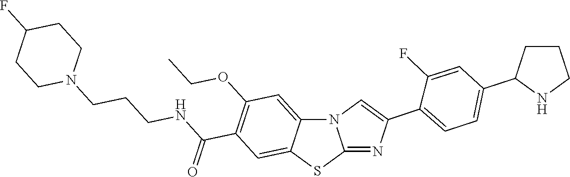 [SMILES: CCOc1cc2c(cc1C(=O)NCCCN1CCC(F)CC1)sc1nc(-c3ccc(C4CCCN4)cc3F)cn12] | 6-ethoxy-2-(2-fluoro-4-(pyrrolidin-2- yl)phenyl)-N-(3-(4-fluoropiperidin-1- yl)propyl)benzo[d]imidazo[2,1- b]thiazole-7-carboxamide |
| 199 | 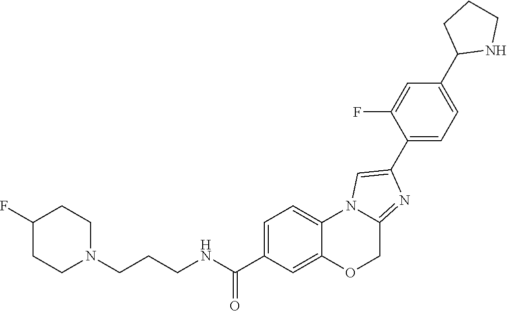 [SMILES: O=C(NCCCN1CCC(F)CC1)c1ccc2c(c1)OCc1nc(-c3ccc(C4CCCN4)cc3F)cn1-2] | 2-(2-fluoro-4-(pyrrolidin-2- yl)phenyl)-N-(3-(4-fluoropiperidin-1- yl)propyl)-4H-benzo[b]imidazo[1,2- d][1,4]oxazine-7-carboxamide |
| 200S | 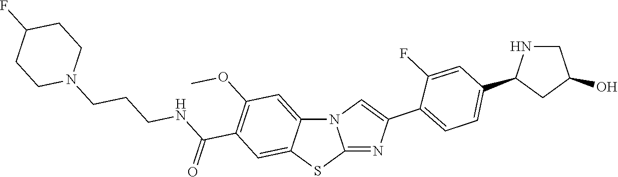 [SMILES: COc1cc2c(cc1C(=O)NCCCN1CCC(F)CC1)sc1nc(-c3ccc([C@@H]4C[C@H](O)CN4)cc3F)cn12] | 2-(2-fluoro-4-((2S,4S)-4- hydroxypyrrolidin-2-yl)phenyl)-N-(3- (4-fluoropiperidin-1-yl)propyl)-6- methoxybenzo[d]imidazo[2,1- b]thiazole-7-carboxamide |
| 200R | 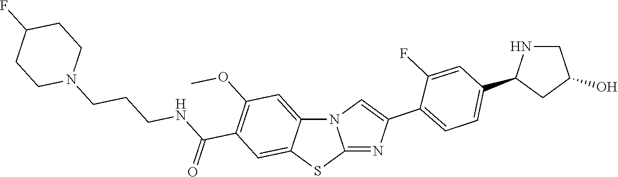 [SMILES: COc1cc2c(cc1C(=O)NCCCN1CCC(F)CC1)sc1nc(-c3ccc([C@@H]4C[C@@H](O)CN4)cc3F)cn12] | 2-(2-fluoro-4-((2S,4R)-4- hydroxypyrrolidin-2-yl)phenyl)-N-(3- (4-fluoropiperidin-1-yl)propyl)-6- methoxybenzo[d]imidazo[2,1- b]thiazole-7-carboxamide |
| 201S | 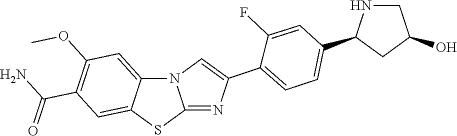 [SMILES: COc1cc2c(cc1C(N)=O)sc1nc(-c3ccc([C@@H]4C[C@H](O)CN4)cc3F)cn12] | 2-(2-fluoro-4-((2S,4S)-4- hydroxypyrrolidin-2-yl)phenyl)-6- methoxybenzo[d]imidazo[2,1- b]thiazole-7-carboxamide |
| 201R | 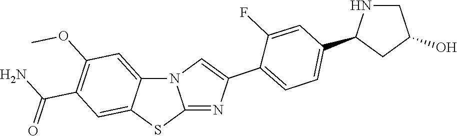 [SMILES: COc1cc2c(cc1C(N)=O)sc1nc(-c3ccc([C@@H]4C[C@@H](O)CN4)cc3F)cn12] | 2-(2-fluoro-4-((2S,4R)-4- hydroxypyrrolidin-2-yl)phenyl)-6- methoxybenzo[d]imidazo[2,1- b]thiazole-7-carboxamide |
| 202S | 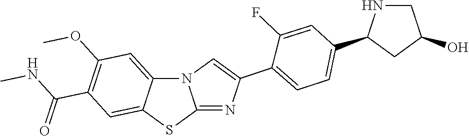 [SMILES: CNC(=O)c1cc2sc3nc(-c4ccc([C@@H]5C[C@H](O)CN5)cc4F)cn3c2cc1OC] | 2-(2-fluoro-4-((2S,4S)-4- hydroxypyrrolidin-2-yl)phenyl)-6- methoxy-N- methylbenzo[d]imidazo[2,1- b]thiazole-7-carboxamide |
| 202R | 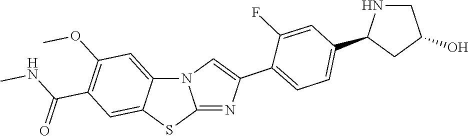 [SMILES: CNC(=O)c1cc2sc3nc(-c4ccc([C@@H]5C[C@@H](O)CN5)cc4F)cn3c2cc1OC] | 2-(2-fluoro-4-((2S,4R)-4- hydroxypyrrolidin-2-yl)phenyl)-6- methoxy-N- methylbenzo[d]imidazo[2,1- b]thiazole-7-carboxamide |
| 203S | 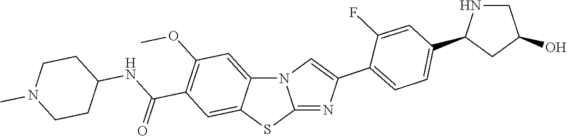 [SMILES: COc1cc2c(cc1C(=O)NC1CCN(C)CC1)sc1nc(-c3ccc([C@@H]4C[C@H](O)CN4)cc3F)cn12] | 2-(2-fluoro-4-((2S,4S)-4- hydroxypyrrolidin-2-yl)phenyl)-6- methoxy-N-(1-methylpiperidin-4- yl)benzo[d]imidazo[2,1-b]thiazole-7- carboxamide |
| 203R | 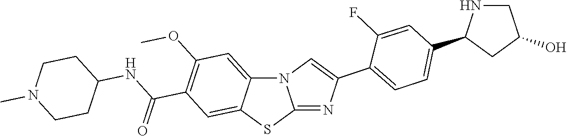 [SMILES: COc1cc2c(cc1C(=O)NC1CCN(C)CC1)sc1nc(-c3ccc([C@@H]4C[C@@H](O)CN4)cc3F)cn12] | 2-(2-fluoro-4-((2S,4R)-4- hydroxypyrrolidin-2-yl)phenyl)-6- methoxy-N-(1-methylpiperidin-4- yl)benzo[d]imidazo[2,1-b]thiazole-7- carboxamide |
| 204 | 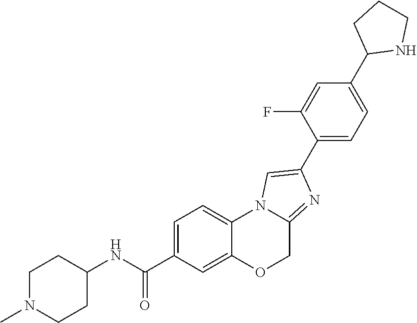 [SMILES: CN1CCC(NC(=O)c2ccc3c(c2)OCc2nc(-c4ccc(C5CCCN5)cc4F)cn2-3)CC1] | 2-(2-fluoro-4-(pyrrolidin-2- yl)phenyl)-N-(1-methylpiperidin-4- yl)-4H-benzo[b]imidazo[1,2- d][1,4]oxazine-7-carboxamide |
| 205 | 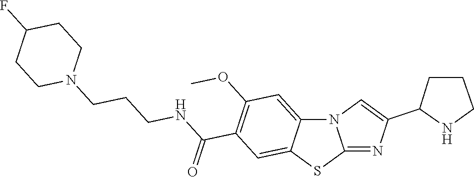 [SMILES: COc1cc2c(cc1C(=O)NCCCN1CCC(F)CC1)sc1nc(C3CCCN3)cn12] | N-(3-(4-fluoropiperidin-1-yl)propyl)- 6-methoxy-2-(pyrrolidin-2- yl)benzo[d]imidazo[2,1-b]thiazole-7- carboxamide |
| 300 | 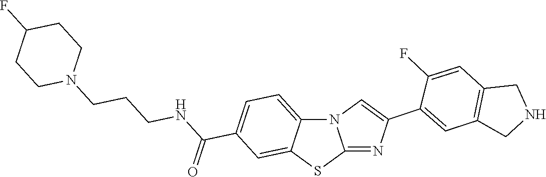 [SMILES: O=C(NCCCN1CCC(F)CC1)c1ccc2c(c1)sc1nc(-c3cc4c(cc3F)CNC4)cn12] | 2-(6-fluoroisoindolin-5-yl)-N-(3-(4- fluoropiperidin-1- yl)propyl)benzo[d]imidazo[2,1- b]thiazole-7-carboxamide |
| 301 | 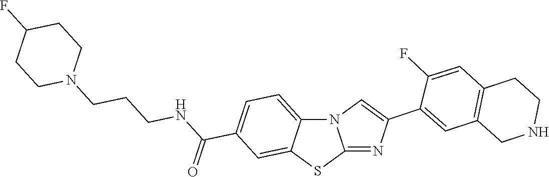 [SMILES: O=C(NCCCN1CCC(F)CC1)c1ccc2c(c1)sc1nc(-c3cc4c(cc3F)CCNC4)cn12] | 2-(6-fluoro-1,2,3,4- tetrahydroisoquinolin-7-yl)-N-(3-(4- fluoropiperidin-1- yl)propyl)benzo[d]imidazo[2,1- b]thiazole-7-carboxamide |
| 302 | 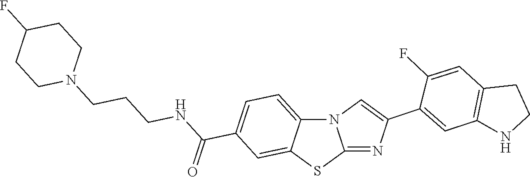 [SMILES: O=C(NCCCN1CCC(F)CC1)c1ccc2c(c1)sc1nc(-c3cc4c(cc3F)CCN4)cn12] | 2-(5-fluoroindolin-6-yl)-N-(3-(4- fluoropiperidin-1- yl)propyl)benzo[d]imidazo[2,1- b]thiazole-7-carboxamide |
| 303 | 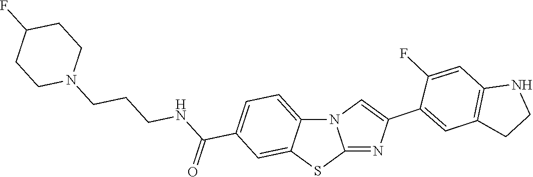 [SMILES: O=C(NCCCN1CCC(F)CC1)c1ccc2c(c1)sc1nc(-c3cc4c(cc3F)NCC4)cn12] | 2-(6-fluoroindolin-5-yl)-N-(3-(4- fluoropiperidin-1- yl)propyl)benzo[d]imidazo[2,1- b]thiazole-7-carboxamide |
| 304 | 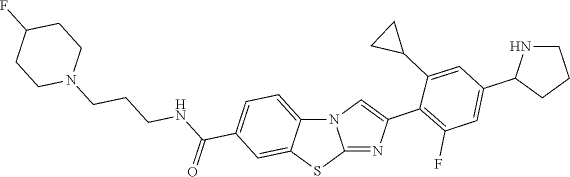 [SMILES: O=C(NCCCN1CCC(F)CC1)c1ccc2c(c1)sc1nc(-c3c(F)cc(C4CCCN4)cc3C3CC3)cn12] | 2-(2-cyclopropyl-6-fluoro-4- (pyrrolidin-2-yl)phenyl)-N-(3-(4- fluoropiperidin-1- yl)propyl)benzo[d]imidazo[2,1- b]thiazole-7-carboxamide |
| 305 | 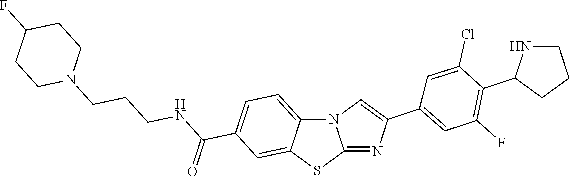 [SMILES: O=C(NCCCN1CCC(F)CC1)c1ccc2c(c1)sc1nc(-c3cc(F)c(C4CCCN4)c(Cl)c3)cn12] | 2-(3-chloro-5-fluoro-4-(pyrrolidin-2- yl)phenyl)-N-(3-(4-fluoropiperidin-1- yl)propyl)benzo[d]imidazo[2,1- b]thiazole-7-carboxamide |
| 306 | 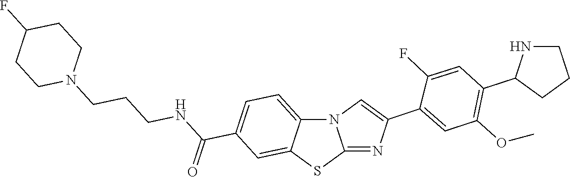 [SMILES: COc1cc(-c2cn3c(n2)sc2cc(C(=O)NCCCN4CCC(F)CC4)ccc23)c(F)cc1C1CCCN1] | 2-(2-fluoro-5-methoxy-4-(pyrrolidin- 2-yl)phenyl)-N-(3-(4-fluoropiperidin- 1-yl)propyl)benzo[d]imidazo[2,1- b]thiazole-7-carboxamide |
| 400S | 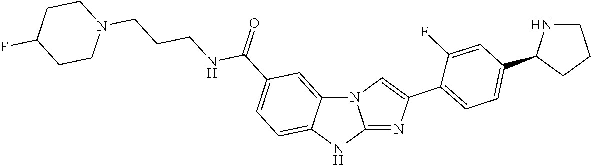 [SMILES: O=C(NCCCN1CCC(F)CC1)c1ccc2[nH]c3nc(-c4ccc([C@@H]5CCCN5)cc4F)cn3c2c1] | (S)-2-(2-fluoro-4-(pyrrolidin-2- yl)phenyl)-N-(3-(4-fluoropiperidin-1- yl)propyl)-9H-benzo[d]imidazo[1,2- a]imidazole-6-carboxamide |
| 400R | 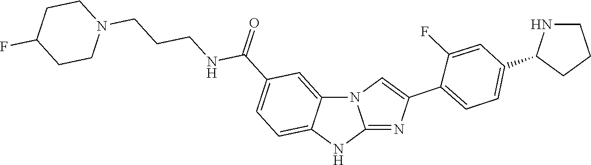 [SMILES: O=C(NCCCN1CCC(F)CC1)c1ccc2[nH]c3nc(-c4ccc([C@H]5CCCN5)cc4F)cn3c2c1] | (R)-2-(2-fluoro-4-(pyrrolidin-2- yl)phenyl)-N-(3-(4-fluoropiperidin-1- yl)propyl)-9H-benzo[d]imidazo[1,2- a]imidazole-6-carboxamide |
| 401 | 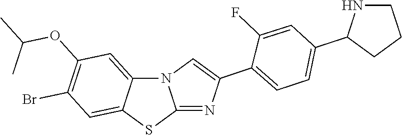 [SMILES: CC(C)Oc1cc2c(cc1Br)sc1nc(-c3ccc(C4CCCN4)cc3F)cn12] | 7-bromo-2-(2-fluoro-4-(pyrrolidin-2- yl)phenyl)-6- isopropoxybenzo[d]imidazo[2,1- b]thiazole |
| 402 | 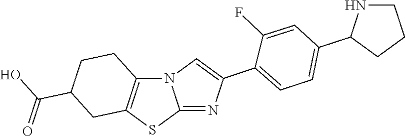 [SMILES: O=C(O)C1CCc2c(sc3nc(-c4ccc(C5CCCN5)cc4F)cn23)C1] | 2-(2-fluoro-4-(pyrrolidin-2- yl)phenyl)-5,6,7,8- tetrahydrobenzo[d]imidazo[2,1- b]thiazole-7-carboxylic acid |
| 403 | 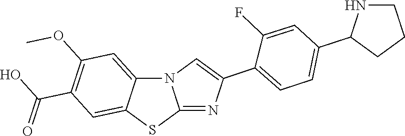 [SMILES: COc1cc2c(cc1C(=O)O)sc1nc(-c3ccc(C4CCCN4)cc3F)cn12] | 2-(2-fluoro-4-(pyrrolidin-2- yl)phenyl)-6- methoxybenzo[d]imidazo[2,1- b]thiazole-7-carboxylic acid |
| 404 | 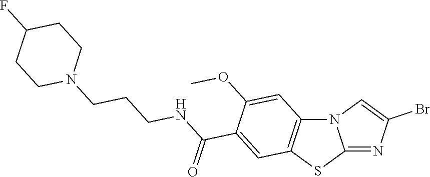 [SMILES: COc1cc2c(cc1C(=O)NCCCN1CCC(F)CC1)sc1nc(Br)cn12] | 2-bromo-N-(3-(4-fluoropiperidin-1- yl)propyl)-6- methoxybenzo[d]imidazo[2,1- b]thiazole-7-carboxamide |
| 408 | 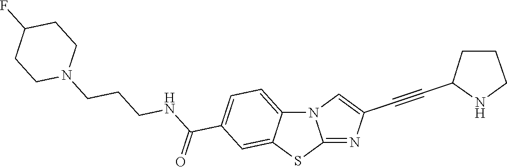 [SMILES: O=C(NCCCN1CCC(F)CC1)c1ccc2c(c1)sc1nc(C#CC3CCCN3)cn12] | N-(3-(4-fluoropiperidin-1-yl)propyl)- 2-(pyrrolidin-2- ylethynyl)benzo[d]imidazo[2,1- b]thiazole-7-carboxamide |
| 409 | 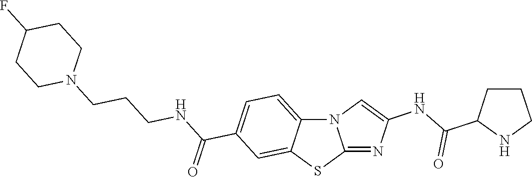 [SMILES: O=C(NCCCN1CCC(F)CC1)c1ccc2c(c1)sc1nc(NC(=O)C3CCCN3)cn12] | N-(3-(4-fluoropiperidin-1-yl)propyl)- 2-(pyrrolidine-2- carboxamido)benzo[d]imidazo[2,1- b]thiazole-7-carboxamide |
| 410 | 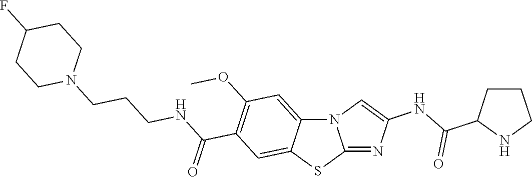 [SMILES: COc1cc2c(cc1C(=O)NCCCN1CCC(F)CC1)sc1nc(NC(=O)C3CCCN3)cn12] | N-(3-(4-fluoropiperidin-1-yl)propyl)- 6-methoxy-2-(pyrrolidine-2- carboxamido)benzo[d]imidazo[2,1- b]thiazole-7-carboxamide |

It is well understood that in structures presented in this invention wherein the carbon atom has less than 4 bonds, H atoms are present to complete the valence of the carbon. It is well understood that in structures presented in this invention wherein the nitrogen atom has less than 3 bonds, H atoms are present to complete the valence of the nitrogen.

In some embodiments, this invention is directed to the compounds listed hereinabove, pharmaceutical compositions and/or method of use thereof, wherein the compound is pharmaceutically acceptable salt, stereoisomer, tautomer, hydrate, N-oxide, reverse amide analog, prodrug, isotopic variant (deuterated analog), PROTAC, pharmaceutical product or any combination thereof. In some embodiments, the compounds are c-MYC mRNA translation modulators. In some embodiments, the compounds are c-MYC mRNA translation inhibitors. In some embodiments, the compounds are c-MYC inhibitors. In various embodiments, the compounds are c-MYC mRNA transcription regulators. In various embodiments, the compounds are any combination of c-MYC mRNA translation modulators, c-MYC mRNA transcription regulators and c-MYC inhibitors.

As used herein, the term “alkyl” can be any straight- or branched-chain alkyl group containing up to about 30 carbons unless otherwise specified. In various embodiments, an alkyl includes C₁-C₅ carbons. In some embodiments, an alkyl includes C₁-C₆ carbons. In some embodiments, an alkyl includes C₁-C₅ carbons. In some embodiments, an alkyl includes C₁-C₅ carbons. In some embodiments, an alkyl includes C₁-C₁₀ carbons. In some embodiments, an alkyl is a C₁-C₁₂ carbons. In some embodiments, an alkyl is a C₁-C₂₀ carbons. In some embodiments, branched alkyl is an alkyl substituted by alkyl side chains of 1 to 5 carbons. In various embodiments, the alkyl group may be unsubstituted. In some embodiments, the alkyl group may be substituted by a halogen, haloalkyl, hydroxyl, alkoxy, carbonyl, amido, alkylamido, dialkylamido, cyano, nitro, CO₂H, amino, alkylamino, dialkylamino, carboxyl, thio, thioalkyl, C₁-C₅ linear or branched haloalkoxy, CF₃, phenyl, halophenyl, (benzyloxy)phenyl, —CH₂CN, NH₂, NH-alkyl, N(alkyl)₂, —OC(O)CF₃, —OCH₂Ph, —NHCO-alkyl, —C(O)Ph, C(O)O-alkyl, C(O)H, —C(O)NH₂ or any combination thereof.

The alkyl group can be a sole substituent, or it can be a component of a larger substituent, such as in an alkoxy, alkoxyalkyl, haloalkyl, arylalkyl, alkylamino, dialkylamino, alkylamido, alkylurea, etc. Preferred alkyl groups are methyl, ethyl, and propyl, and thus halomethyl, dihalomethyl, trihalomethyl, haloethyl, dihaloethyl, trihaloethyl, halopropyl, dihalopropyl, trihalopropyl, methoxy, ethoxy, propoxy, arylmethyl, arylethyl, arylpropyl, methylamino, ethylamino, propylamino, dimethylamino, diethylamino, methylamido, acetamido, propylamido, halomethylamido, haloethylamido, halopropylamido, methyl-urea, ethyl-urea, propyl-urea, 2, 3, or 4-CH₂—C₆H₄—Cl, C(OH)(CH₃)(Ph), etc.

As used herein, the term “aryl” refers to any aromatic ring that is directly bonded to another group and can be either substituted or unsubstituted. The aryl group can be a sole substituent, or the aryl group can be a component of a larger substituent, such as in an arylalkyl, arylamino, arylamido, etc. In some embodiments, the term aryl according to this invention, includes also heteroaryl. Exemplary aryl groups include, without limitation, phenyl, tolyl, xylyl, furanyl, naphthyl, pyridinyl, pyrimidinyl, pyridazinyl, pyrazinyl, triazinyl, thiazolyl, oxazolyl, isooxazolyl, pyrazolyl, imidazolyl, thiophene-yl, pyrrolyl, indolyl, phenylmethyl, phenylethyl, phenylamino, phenylamido, 3-methyl-4H-1,2,4-triazolyl, oxadiazolyl, 5-methyl-1,2,4-oxadiazolyl, isothiazolyl, thiadiazolyl, triazolyl, etc. Substitutions include but are not limited to: F, Cl, Br, I, C₁-C₅ linear or branched alkyl, C₁-C₅ linear or branched haloalkyl, C₁-C₅ linear or branched alkoxy, C₁-C₅ linear or branched haloalkoxy, CF₃, phenyl, halophenyl, CN, NO₂, —CH₂CN, NH₂, NH-alkyl, N(alkyl)₂, hydroxyl, —OC(O)CF₃, —OCH₂Ph, —NHCO-alkyl, COOH, —C(O)Ph, C(O)O-alkyl, C(O)H, —C(O)NH₂ or any combination thereof.

As used herein, the term “alkoxy” refers to an ether group substituted by an alkyl group as defined above. Alkoxy refers both to linear and to branched alkoxy groups. Nonlimiting examples of alkoxy groups are methoxy, ethoxy, propoxy, iso-propoxy, tert-butoxy.

As used herein, the term “aminoalkyl” refers to an amine group substituted by an alkyl group as defined above. Aminoalkyl refers to monoalkylamine, dialkylamine or trialkylamine. Nonlimiting examples of aminoalkyl groups are —N(Me)₂, —NHMe, —NH₃.

A “haloalkyl” group refers, in some embodiments, to an alkyl group as defined above, which is substituted by one or more halogen atoms, e.g. by F, Cl, Br or I. The term “haloalkyl” include but is not limited to fluoroalkyl, i.e., to an alkyl group bearing at least one fluorine atom. Nonlimiting examples of haloalkyl groups are CF₃, CF₂CF₃, CF₂CH₃, CH₂CF₃, CF₂CH₂CH₃, CH₂CH₂CF₃, CF₂CH(CH₃)₂ and CF(CH₃)—CH(CH₃)₂.

A “halophenyl” group refers, in some embodiments, to a phenyl substitutent which is substituted by one or more halogen atoms, e.g. by F, Cl, Br or I. In one embodiment, the halophenyl is 4-chlorophenyl.

An “alkoxyalkyl” group refers, in some embodiments, to an alkyl group as defined above, which is substituted by alkoxy group as defined above, e.g. by methoxy, ethoxy, propoxy, i-propoxy, t-butoxy etc. Nonlimiting examples of alkoxyalkyl groups are —CH₂—O—CH₃, —CH₂—O—CH(CH₃)₂, —CH₂—O—C(CH₃)₃, —CH₂—CH₂—O—CH₃, —CH₂—CH₂—O—CH(CH₃)₂, —CH₂—CH₂—O—C(CH₃)₃.

A “cycloalkyl” or “carbocyclic” group refers, in various embodiments, to a ring structure comprising carbon atoms as ring atoms, which may be either saturated or unsaturated, substituted or unsubstituted, single or fused. In some embodiments the cycloalkyl is a 3-10 membered ring. In some embodiments the cycloalkyl is a 3-12 membered ring. In some embodiments the cycloalkyl is a 6 membered ring. In some embodiments the cycloalkyl is a 5-7 membered ring. In some embodiments the cycloalkyl is a 3-8 membered ring. In some embodiments, the cycloalkyl group may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio, thioalkyl, C₁-C₅ linear or branched haloalkoxy, CF₃, phenyl, halophenyl, (benzyloxy)phenyl, —CH₂CN, NH₂, NH-alkyl, N(alkyl)₂, —OC(O)CF₃, —OCH₂Ph, —NHCO-alkyl, —C(O)Ph, C(O)O-alkyl, C(O)H, —C(O)NH₂ or any combination thereof. In some embodiments, the cycloalkyl ring may be fused to another saturated or unsaturated cycloalkyl or heterocyclic 3-8 membered ring. In some embodiments, the cycloalkyl ring is a saturated ring. In some embodiments, the cycloalkyl ring is an unsaturated ring. Non limiting examples of a cycloalkyl group comprise cyclohexyl, cyclohexenyl, cyclopropyl, cyclopropenyl, cyclopentyl, cyclopentenyl, cyclopentadienyl, cyclobutyl, cyclobutenyl, cycloctyl, cycloctadienyl (COD), cycloctaene (COE) etc.

A “heterocycle” or “heterocyclic” group refers, in various embodiments, to a ring structure comprising in addition to carbon atoms, sulfur, oxygen, nitrogen or any combination thereof, as part of the ring. A “heteroaromatic ring” refers in various embodiments, to an aromatic ring structure comprising in addition to carbon atoms, sulfur, oxygen, nitrogen or any combination thereof, as part of the ring. In some embodiments the heterocycle or heteroaromatic ring is a 3-10 membered ring. In some embodiments the heterocycle or heteroaromatic ring is a 3-12 membered ring. In some embodiments the heterocycle or heteroaromatic ring is a 6 membered ring. In some embodiments the heterocycle or heteroaromatic ring is a 5-7 membered ring. In some embodiments the heterocycle or heteroaromatic ring is a 3-8 membered ring. In some embodiments, the heterocycle group or heteroaromatic ring may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio, thioalkyl, C₁-C₅ linear or branched haloalkoxy, CF₃, phenyl, halophenyl, (benzyloxy)phenyl, —CH₂CN, NH₂, NH-alkyl, N(alkyl)₂, —OC(O)CF₃, —OCH₂Ph, —NHCO-alkyl, —C(O)Ph, C(O)O-alkyl, C(O)H, —C(O)NH₂ or any combination thereof. In some embodiments, the heterocycle ring or heteroaromatic ring may be fused to another saturated or unsaturated cycloalkyl or heterocyclic 3-8 membered ring. In some embodiments, the heterocyclic ring is a saturated ring. In some embodiments, the heterocyclic ring is an unsaturated ring. Non limiting examples of a heterocyclic ring or heteroaromatic ring systems comprise pyridine, piperidine, morpholine, piperazine, thiophene, pyrrole, benzodioxole, benzofuran-2(3H)-one, benzo[d][1,3]dioxole, indole, oxazole, isoxazole, imidazole and 1-methylimidazole, furane, triazole, pyrimidine, pyrazine, oxacyclobutane (1 or 2-oxacyclobutane), naphthalene, tetrahydrothiophene 1,1-dioxide, thiazole, benzimidazole, piperidine, 1-methylpiperidine, isoquinoline, 1,3-dihydroisobenzofuran, benzofuran, 3-methyl-4H-1,2,4-triazole, oxadiazolyl, 5-methyl-1,2,4-oxadiazole, pyrazole, isothiazole, thiadiazole, tetrahydrofurane, oxazolone, oxazolidone, thiazolone, isothiazolinone, isoxazolidinone, imidazolidinone, pyrazolone, 2H-pyrrol-2-one, furanone, thiophenone, thiane 1,1-dioxide, triazolopyrimidine, 6,7-dihydro-5H-pyrazolo[5,1-b][1,3]oxazine or indole.

In some embodiments, “heterocyclic ring” according to this invention refers to substituted or unsubstituted, 3 to 8 membered, saturated, unsaturated or aromatic, single, fused or spiro rings, which comprise at least one heteroatom selected from: N, O or S. In some embodiments, the heterocyclic ring may be substituted, unsubstituted, saturated, unsaturated, aromatic, single, fused or spiro ring; each represent a separate embodiment according to this invention. The heterocyclic ring(s) may be 3-10; 3-9; 3-8; 3-7; 3-6; 3-5; 4-6; 4-7; 4-8; 4-9; 5-6; 5-7; 5-8; 5-10 or 5-9 membered ring(s); each represents a separate embodiment according to this invention. Examples of heterocyclic rings include, but to limited to: pyran, tetrahydropyran, pyrrazole, imidazole, furan, tetrahydrofuran, dioxane, azetidine, pyridine, pyridazine, pyrimidine, piperidine, piperazine, triazole, oxadiazole, tetrahydrofuran (THF), tetrahydrofurane, morpholine, thiomorpholine 1,1-dioxide, oxa-azaspirodecane, azaspiroheptane, 5-azaspiro[2.4]heptane, 2-azaspiro[3.3]heptane, oxa-azaspiroheptane, 2-oxa-6-azaspiro[3.3]heptane pyrrol, pyrrolidine, pyrrolidine-2-one, 2-oxo-pyrrolidine, pyrrolidinone, quinuclidine, oxetane, azepane, azepan-2-one, azabicyclohexane, 2-azabicyclo[2.1.1]hexane, 3-azabicyclo[3.1.0]hexane, 1-oxa-8-azaspiro[4.5]decane, diazabicyclo[2.2.1]heptane, 2,5-diazabicyclo[2.2.1]heptane. In some embodiments, the heterocyclic ring may be further substituted with at least one group selected from: F, Cl, Br, I, CF₃, R₂₀ as defined hereinbelow, C₁-C₅ linear or branched alkyl (e.g., methyl, ethyl, propyl), alkyleneamine (e.g., CH₂—NH₂), C₁-C₅ linear or branched haloalkyl, OH, alkoxy (e.g., OCH₃), alkylene-OH (e.g., CH₂—OH), amide, alkylene-amide (e.g., CH₂—C(O)NH₂), C(O)-heterocyclic ring, amine (e.g., NH₂), alkylamine (e.g., NH(CH₃)), dialkylamine (e.g., N(CH₃)₂), CF₃, aryl, phenyl, halophenyl, heteroaryl, C₃-C₅ cycloalkyl (e.g., cyclopropyl), saturated, unsaturated, aromatic, single fused or spiral 3-8 membered heterocyclic ring, CN, and NO₂; each is a separate embodiment according to this invention.

In some embodiments, “single or fused saturated, unsaturated or aromatic heterocyclic ring” or “saturated, unsaturated, aromatic, single, fused or spiro heterocyclic ring” can be any such ring(s), which comprise at least one heteroatom selected from: N, O or S, including but not limited to: pyridinyl, (2-, 3-, and 4-pyridinyl), quinolinyl, pyrimidinyl, pyridazinyl, pyrazinyl, triazinyl, tetrazinyl, thiazolyl, isothiazolyl, oxazolyl, isoxazolyl, imidazolyl, 1-methylimidazole, pyrazolyl, pyrrolyl, furanyl, thiophene-yl, quinolinyl, isoquinolinyl, 2,3-dihydroindenyl, indenyl, tetrahydronaphthyl, 3,4-dihydro-2H-benzo[b][1,4]dioxepine, benzodioxolyl, benzo[d][1,3]dioxole, tetrahydronaphthyl, indolyl, 1H-indole, isoindolyl, anthracenyl, benzimidazolyl, 2,3-dihydro-1H-benzo[d]imidazolyl, indazolyl, 2H-indazole, triazolyl, 4,5,6,7-tetrahydro-2H-indazole, 3H-indol-3-one, purinyl, benzoxazolyl, 1,3-benzoxazolyl, benzisoxazolyl, benzothiazolyl, 1,3-benzothiazole, 4,5,6,7-tetrahydro-1,3-benzothiazole, quinazolinyl, quinoxalinyl, 1,2,3,4-tetrahydroquinoxaline, 1-(pyridin-1(2H)-yl)ethanone, cinnolinyl, phthalazinyl, quinolinyl, isoquinolinyl, acridinyl, benzofuranyl, 1-benzofuran, isobenzofuranyl, benzofuran-2(3H)-one, benzothiophenyl, benzoxadiazole, benzo[c][1,2,5]oxadiazolyl, benzo[c]thiophenyl, benzodioxolyl, thiadiazolyl, [1,3]oxazolo[4,5-b]pyridine, 1,2,3-, 1,2,4-, 1,2,5- or 1,3,4-oxadiazolyl, imidazo[2,1-b][1,3]thiazole, 4H,5H,6H-cyclopenta[d][1,3]thiazole, 5H,6H,7H,8H-imidazo[1,2-a]pyridine, 7-oxo-6H,7H-[1,3]thiazolo[4,5-d]pyrimidine, [1,3]thiazolo[5,4-b]pyridine, 2H,3H-imidazo[2,1-b][1,3]thiazole, thieno[3,2-d]pyrimidin-4(3H)-one, 4-oxo-4H-thieno[3,2-d][1,3]thiazin, imidazo[1,2-a]pyridine, 1H-imidazo[4,5-b]pyridine, 1H-imidazo[4,5-c]pyridine, 3H-imidazo[4,5-c]pyridine, pyrazolo[1,5-a]pyridine, imidazo[1,2-a]pyrazine, imidazo[1,2-a]pyrimidine, 1H-pyrrolo[2,3-b]pyridine, pyrido[2,3-b]pyrazine, pyrido[2,3-b]pyrazin-3(4H)-one, 4H-thieno[3,2-b]pyrrole, quinoxalin-2(1H)-one, 1H-pyrrolo[3,2-b]pyridine, 7H-pyrrolo[2,3-d]pyrimidine, oxazolo[5,4-b]pyridine, thiazolo[5,4-b]pyridine, thieno[3,2-c]pyridine, 3-methyl-4H-1,2,4-triazole, 5-methyl-1,2,4-oxadiazole etc. In some embodiments, the heterocyclic ring according to this invention includes: pyran, tetrahydropyran, pyrrazole, imidazole, furan, tetrahydrofuran, dioxane, oxetane, azetidine, pyridine, pyridazine, pyrimidine, piperidine, piperazine, triazole, oxadiazole, tetrahydrofuran (THF), tetrahydrofurane, morpholine, thiomorpholine 1,1-dioxide, oxa-azaspirodecane, azaspiroheptane, 5-azaspiro[2.4]heptane, 2-azaspiro[3.3]heptane, oxa-azaspiroheptane, pyrrol, pyrrolidine, pyrrolidine-2-one, 2-oxo-pyrrolidine, pyrrolidinone, quinuclidine, azepane, azepan-2-one, azabicyclohexane, 2-azabicyclo[2.1.1]hexane, 3-azabicyclo[3.1.0]hexane, 1-oxa-8-azaspiro[4.5]decane, and/or diazabicyclo[2.2.1]heptane; each represent a separate embodiment according to this invention. In some embodiments, the heterocyclic ring may be further substituted with at least one group selected from: F, Cl, Br, I, CF₃, R₂₀ as defined hereinbelow, C₁-C₅ linear or branched alkyl (e.g., methyl, ethyl, propyl), alkyleneamine (e.g., CH₂—NH₂), C₁-C₅ linear or branched haloalkyl, OH, alkoxy (e.g., OCH₃), alkylene-OH (e.g., CH₂—OH), amide, alkylene-amide (e.g., CH₂—C(O)NH₂), C(O)-heterocyclic ring, amine (e.g., NH₂), alkylamine (e.g., NH(CH₃)), dialkylamine (e.g., N(CH₃)₂), CF₃, aryl, phenyl, halophenyl, heteroaryl, C₃-C₅ cycloalkyl (e.g., cyclopropyl), saturated, unsaturated, aromatice, single fused or spiral 3-8 membered heterocyclic ring, CN, and NO₂; each is a separate embodiment according to this invention.

In various embodiments, this invention provides a compound of this invention or its isomer, metabolite, pharmaceutically acceptable salt, pharmaceutical product, tautomer, hydrate, N-oxide, reverse amide analog, prodrug, isotopic variant (deuterated analog), PROTAC, polymorph, or crystal or combinations thereof. In various embodiments, this invention provides an isomer of the compound of this invention. In some embodiments, this invention provides a metabolite of the compound of this invention. In some embodiments, this invention provides a pharmaceutically acceptable salt of the compound of this invention. In some embodiments, this invention provides a pharmaceutical product of the compound of this invention. In some embodiments, this invention provides a tautomer of the compound of this invention. In some embodiments, this invention provides a hydrate of the compound of this invention. In some embodiments, this invention provides an N-oxide of the compound of this invention. In some embodiments, this invention provides a reverse amide analog of the compound of this invention. In some embodiments, “reverse amide analog” refers to acyclic amides or amides of acyclic amines. In some embodiments, this invention provides a prodrug of the compound of this invention. In some embodiments, this invention provides an isotopic variant (including but not limited to deuterated analog) of the compound of this invention. In some embodiments, this invention provides a PROTAC (Proteolysis targeting chimera) of the compound of this invention. In some embodiments, this invention provides a polymorph of the compound of this invention. In some embodiments, this invention provides a crystal of the compound of this invention. In some embodiments, this invention provides composition comprising a compound of this invention, as described herein, or, In some embodiments, a combination of an isomer, metabolite, pharmaceutically acceptable salt, pharmaceutical product, tautomer, hydrate, N-oxide, reverse amide analog, prodrug, isotopic variant (deuterated analog), PROTAC, polymorph, or crystal of the compound of this invention.

In various embodiments, the term “isomer” includes, but is not limited to, stereoisomers including optical isomers and analogs, structural isomers and analogs, conformational isomers and analogs, and the like. In some embodiments, the isomer is a stereoisomer. In another embodiment, the isomer is an optical isomer.

Certain compounds of the present invention may exist in particular geometric or stereoisomeric forms. The present invention contemplates all such compounds, including cis- and trans-isomers, R- and S-enantiomers, diastereomers, the racemic mixtures thereof, and other mixtures thereof, as falling within the scope of the invention. Additional asymmetric carbon atoms may be present in a substituent such as an alkyl group. All such isomers, as well as mixtures thereof, are included in this invention.

In various embodiments, this invention encompasses the use of various stereoisomers of the compounds of the invention. It will be appreciated by those skilled in the art that the compounds of the present invention may contain at least one chiral center. Accordingly, the compounds used in the methods of the present invention may exist in, and be isolated in, optically-active or racemic forms. The compounds according to this invention may further exist as stereoisomers which may be also optically-active isomers (e.g., enantiomers such as (R) or (S)), as enantiomerically enriched mixtures, racemic mixtures, or as single diastereomers, diastereomeric mixtures, or any other stereoisomers, including but not limited to: (R)(R), (R)(S), (S)(S), (S)(R), (R)(R)(R), (R)(R)(S), (R)(S)(R), (S)(R)(R), (R)(S)(S), (S)(R)(S), (S)(S)(R) or (S)(S)(S) stereoisomers. Some compounds may also exhibit polymorphism. It is to be understood that the present invention encompasses any racemic, optically-active, polymorphic, or stereroisomeric form, or mixtures thereof, which form possesses properties useful in the treatment of the various conditions described herein.

It is well known in the art how to prepare optically active forms (for example, by resolution of the racemic form by recrystallization techniques, by synthesis from optically-active starting materials, by chiral synthesis, or by chromatographic separation using a chiral stationary phase).

The compounds of the present invention can also be present in the form of a racemic mixture, containing substantially equivalent amounts of stereoisomers. In some embodiments, the compounds of the present invention can be prepared or otherwise isolated, using known procedures, to obtain a stereoisomer substantially free of its corresponding stereoisomer (i.e., substantially pure). By substantially pure, it is intended that a stereoisomer is at least about 80% pure, more preferably at least about 95% pure, even more preferably at least about 98% pure, most preferably at least about 99% pure. In various embodiments, the compound according to the invention comprises a substantially pure stereoisomer. In some embodiments, the substantially pure stereoisomer is at least 70%; 75%; 80%; 85%; 90%; 93%; 95%; 97%; 98%; 99%; 99.5% pure; each represents a separate embodiment according to this invention.

In various embodiments, the compound comprises a single stereoisomer in a purity of >80%; >85%; >90%; >91%; >92%; >93%; >94%; >95%; >96%; >97%; >98%; >99%; >99.5% enantiomeric excess (ee); each represents a separate embodiment according to this invention. In various embodiments, the compound comprises a single stereoisomer in a purity >80%; >85%; >90%; >91%; >92%; >93%; >94%; >95%; >96%; >97%; >98%; >99%; >99.5% enantiomeric ratio (er); each represents a separate embodiment according to this invention. In various embodiments, the compound comprises a single stereoisomer in a purity higher than 80%; 85%; 90%; 91%; 92%; 93%; 94%; 95%; 96%; 97%; 98%; 99%; 99.5%; each represents a separate embodiment according to this invention.

In various embodiments, the compound is a substantially pure single enantiomer. In various embodiments, the compound comprises a mixture of enantiomers. In various embodiments, the compound is a racemate.

In various embodiments, the compound has two chiral centers. In various embodiments, the compound comprises a mixture of stereoisomers. In various embodiments, the compound comprises a mixture of 2, 3, or 4 stereoisomers; each represents a separate embodiment according to this invention. In various embodiments, the compound is a single stereoisomer. In various embodiments, the compound is a substantially pure single stereoisomer. In various embodiments, the substantially pure stereoisomer has at least 80%, 85%, 90%, 95%, 97%, 98%, 99% purity; each represents a separate embodiment according to this invention. In various embodiments, the compound is the substantially pure RR stereoisomer. In various embodiments, the compound is the substantially pure SS stereoisomer. In various embodiments, the compound is the substantially pure RS stereoisomer. In various embodiments, the compound is the substantially pure SR stereoisomer.

Compounds of the present invention can also be in the form of a hydrate, which means that the compound further includes a stoichiometric or non-stoichiometric amount of water bound by non-covalent intermolecular forces.

As used herein, when some chemical functional group (e.g., alkyl or aryl) is said to be “substituted”, it is herein defined that one or more substitutions are possible. In some embodiments, the term “substituted” according to this invention, refers to but is not limited to at least one group selected from: halogen, substituted or unsubstituted C₁-C₅ linear or branched alkyl (e.g., methyl, ethyl, propyl, isopropyl, CH(Me)CH₂—OMe), OH, C₁-C₅ linear or branched alkyl-OH (e.g., C(CH₃)₂CH₂—OH, CH₂CH₂—OH), linear, branched or cyclic alkoxy (e.g., OMe, oxetane), amide (e.g., C(O)N(R)₂, C(O)-pyrrolidine, C(O)-piperidine, N(R)₂, NH(R₁₀), N(R₁₀)(R₁₁), (e.g., N(CH₃)₂, NH₂), CF₃, aryl, phenyl, heteroaryl, substituted or unsubstituted C₃-C₅ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

Compounds of the present invention may exist in the form of one or more of the possible tautomers and depending on the conditions it may be possible to separate some or all of the tautomers into individual and distinct entities. It is to be understood that all of the possible tautomers, including all additional enol and keto tautomers and/or isomers are hereby covered. For example, the following tautomers, but not limited to these, are included:

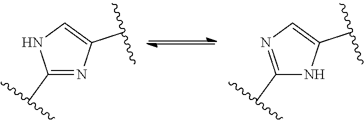 [SMILES: *c1c[nH]c(*)n1 ; *c1cnc(*)[nH]1]

### Tautomerization of the Pyrazolone Ring:

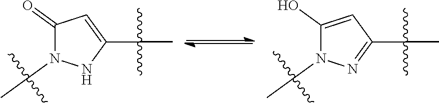 [SMILES: *c1cc(=O)n(*)[nH]1 ; *c1cc(O)n(*)n1]

The invention includes “pharmaceutically acceptable salts” of the compounds of this invention, which may be produced, by reaction of a compound of this invention with an acid or base. Certain compounds, particularly those possessing acid or basic groups, can also be in the form of a salt, preferably a pharmaceutically acceptable salt. The term “pharmaceutically acceptable salt” refers to those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. Other salts are known to those of skill in the art and can readily be adapted for use in accordance with the present invention.

Suitable pharmaceutically acceptable salts of amines of the compounds of this invention may be prepared from an inorganic acid or from an organic acid. In various embodiments, examples of inorganic salts of amines are bisulfates, borates, bromides, chlorides, hemisulfates, hydrobromates, hydrochlorates, 2-hydroxyethylsulfonates (hydroxyethanesulfonates), iodates, iodides, isethionates, nitrates, persulfates, phosphate, sulfates, sulfamates, sulfanilates, sulfonic acids (alkylsulfonates, arylsulfonates, halogen substituted alkylsulfonates, halogen substituted arylsulfonates), sulfonates and thiocyanates.

In various embodiments, examples of organic salts of amines may be selected from aliphatic, cycloaliphatic, aromatic, araliphatic, heterocyclic, carboxylic and sulfonic classes of organic acids, examples of which are acetates, arginines, aspartates, ascorbates, adipates, anthranilates, alkane carboxylates, substituted alkane carboxylates, alginates, benzenesulfonates, benzoates, bisulfates, butyrates, bicarbonates, bitartrates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthates, ethanesulfonates, edetates, edisylates, estolates, esylates, fumarates, formates, fluorides, galacturonates gluconates, glutamates, glycolates, glucorate, glucoheptanoates, glycerophosphates, gluceptates, glycollylarsanilates, glutarates, glutamate, heptanoates, hexanoates, hydroxymaleates, hydroxycarboxlic acids, hexylresorcinates, hydroxybenzoates, hydroxynaphthoates, hydrofluorates, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, naphthalenesulfonates, 2-naphthalenesulfonates, nicotinates, nitrates, napsylates, N-methylglucamines, oxalates, octanoates, oleates, pamoates, phenylacetates, picrates, phenylbenzoates, pivalates, propionates, phthalates, phenylacetate, pectinates, phenylpropionates, palmitates, pantothenates, polygalacturates, pyruvates, quinates, salicylates, succinates, stearates, sulfanilate, subacetates, tartrates, theophyllineacetates, p-toluenesulfonates (tosylates), trifluoroacetates, terephthalates, tannates, teoclates, trihaloacetates, triethiodide, tricarboxylates, undecanoates and valerates.

In various embodiments, examples of inorganic salts of carboxylic acids or hydroxyls may be selected from ammonium, alkali metals to include lithium, sodium, potassium, cesium; alkaline earth metals to include calcium, magnesium, aluminium; zinc, barium, cholines, quaternary ammoniums.

In some embodiments, examples of organic salts of carboxylic acids or hydroxyl may be selected from arginine, organic amines to include aliphatic organic amines, alicyclic organic amines, aromatic organic amines, benzathines, t-butylamines, benethamines (N-benzylphenethylamine), dicyclohexylamines, dimethylamines, diethanolamines, ethanolamines, ethylenediamines, hydrabamines, imidazoles, lysines, methylamines, meglumines, N-methyl-D-glucamines, N,N′-dibenzylethylenediamines, nicotinamides, organic amines, ornithines, pyridines, picolies, piperazines, procain, tris(hydroxymethyl)methylamines, triethylamines, triethanolamines, trimethylamines, tromethamines and ureas.

In various embodiments, the salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of a existing salt for another ion or suitable ion-exchange resin.

### Pharmaceutical Composition

Another aspect of the present invention relates to a pharmaceutical composition including a pharmaceutically acceptable carrier and a compound according to the aspects of the present invention. The pharmaceutical composition can contain one or more of the above-identified compounds of the present invention. Typically, the pharmaceutical composition of the present invention will include a compound of the present invention or its pharmaceutically acceptable salt, as well as a pharmaceutically acceptable carrier. The term “pharmaceutically acceptable carrier” refers to any suitable adjuvants, carriers, excipients, or stabilizers, and can be in solid or liquid form such as, tablets, capsules, powders, solutions, suspensions, or emulsions.

Typically, the composition will contain from about 0.01 to 99 percent, preferably from about 20 to 75 percent of active compound(s), together with the adjuvants, carriers and/or excipients. While individual needs may vary, determination of optimal ranges of effective amounts of each component is within the skill of the art. Typical dosages comprise about 0.01 to about 100 mg/kg body wt. The preferred dosages comprise about 0.1 to about 100 mg/kg body wt. The most preferred dosages comprise about 1 to about 100 mg/kg body wt. Treatment regimen for the administration of the compounds of the present invention can also be determined readily by those with ordinary skill in art. That is, the frequency of administration and size of the dose can be established by routine optimization, preferably while minimizing any side effects.

The solid unit dosage forms can be of the conventional type. The solid form can be a capsule and the like, such as an ordinary gelatin type containing the compounds of the present invention and a carrier, for example, lubricants and inert fillers such as, lactose, sucrose, or cornstarch. In some embodiments, these compounds are tabulated with conventional tablet bases such as lactose, sucrose, or cornstarch in combination with binders like acacia, cornstarch, or gelatin, disintegrating agents, such as cornstarch, potato starch, or alginic acid, and a lubricant, like stearic acid or magnesium stearate.

The tablets, capsules, and the like can also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it can contain, in addition to materials of the above type, a liquid carrier such as a fatty oil.

Various other materials may be present as coatings or to modify the physical form of the dosage unit. For instance, tablets can be coated with shellac, sugar, or both. A syrup can contain, in addition to active ingredient, sucrose as a sweetening agent, methyl and propylparabens as preservatives, a dye, and flavoring such as cherry or orange flavor.

For oral therapeutic administration, these active compounds can be incorporated with excipients and used in the form of tablets, capsules, elixirs, suspensions, syrups, and the like. Such compositions and preparations should contain at least 0.10% of active compound. The percentage of the compound in these compositions can, of course, be varied and can conveniently be between about 2% to about 60% of the weight of the unit. The amount of active compound in such therapeutically useful compositions is such that a suitable dosage will be obtained. Preferred compositions according to the present invention are prepared so that an oral dosage unit contains between about 1 mg and 800 mg of active compound.

The active compounds of the present invention may be orally administered, for example, with an inert diluent, or with an assimilable edible carrier, or they can be enclosed in hard- or soft-shell capsules, or they can be compressed into tablets, or they can be incorporated directly with the food of the diet.

The pharmaceutical forms suitable for injectable use include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds or pharmaceutical compositions of the present invention may also be administered in injectable dosages by solution or suspension of these materials in a physiologically acceptable diluent with a pharmaceutical adjuvant, carrier or excipient. Such adjuvants, carriers and/or excipients include, but are not limited to, sterile liquids, such as water and oils, with or without the addition of a surfactant and other pharmaceutically and physiologically acceptable components. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

These active compounds may also be administered parenterally. Solutions or suspensions of these active compounds can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

In various embodiments, the compounds of this invention are administered in combination with an anti-cancer therapy. Examples of such therapies include but are not limited to: chemotherapy, immunotherapy, radiotherapy, biological therapy, surgical intervention, and combinations thereof. In various embodiments, the compound is administered in combination with an anti-cancer agent by administering the compounds as herein described, alone or in combination with other agents.

When administering the compounds of the present invention, they can be administered systemically or, alternatively, they can be administered directly to a specific site where cancer is present. Thus, administering can be accomplished in any manner effective for delivering the compounds or the pharmaceutical compositions to the cancerous cells. Exemplary modes of administration include, without limitation, administering the compounds or compositions orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes.

### Biological Activity

In various embodiments, the invention provides compounds and compositions, including any embodiment described herein, for use in any of the methods of this invention. In various embodiments, use of a compound of this invention or a composition comprising the same, will have utility in inhibiting, suppressing, enhancing, or stimulating a desired response in a subject, as will be understood by one skilled in the art. In some embodiments, the compositions may further comprise additional active ingredients, whose activity is useful for the particular application for which the compound of this invention is being administered.

The invention relates to the treatment, inhibition, and reduction of cancer, employing the use of a compound according to this invention or a pharmaceutically acceptable salt thereof. Accordingly, in various embodiments, this invention is directed to a method of treating, suppressing, reducing the severity, reducing the risk of developing or inhibiting cancer in a subject, comprising administering a compound according to this invention, to a subject suffering from cancer under conditions effective to treat, suppress, reduce the severity, reduce the risk of developing, or inhibit cancer in said subject. In some embodiments, the compound is a c-MYC mRNA translation modulator. In some embodiments, the compound is a c-MYC mRNA translation inhibitor. In some embodiments, the compound is a c-MYC inhibitor. In some embodiments, the compound is a c-MYC mRNA transcription regulator. In some embodiments, the compound is any combination of a c-MYC mRNA translation modulator, a c-MYC mRNA transcription regulator and a c-MYC inhibitor. In some embodiments, the compound is any one of the compounds listed in Table 1; each compound represents a separate embodiment according to this invention. In some embodiments, the cancer is early cancer. In some embodiments, the cancer is advanced cancer. In some embodiments, the cancer is invasive cancer. In some embodiments, the cancer is metastatic cancer. In some embodiments, the cancer is drug resistant cancer.

In some embodiments, the cancer is selected from the following list: bladder cancer (urothelial carcinoma), myelodysplasia, breast cancer, cervix cancer, endometrium cancer, esophagus cancer, head and neck cancer (squamous cell carcinoma), kidney cancer (e.g., renal cell carcinoma, clear cell renal cell carcinoma), liver cancer (hepatocellular carcinoma), lung cancer (e.g., metastatic, non-small cell, NSCLC, squamous cell carcinoma, small cell (SCLC)), metastatic cancer (e.g., to brain), nasopharynx cancer, solid tumor cancer, stomach cancer, adrenocortical carcinoma, Glioblastoma multiforme, acute myeloid leukemia, chronic lymphocytic leukemia, lymphoma (e.g., Hodgkin's (classical), diffuse large B-cell, primary central nervous system), malignant melanoma, uveal melanoma, meningioma, multiple myeloma, breast cancer, metastatic breast cancer, anus cancer (e.g. squamous cell), biliary cancer, bladder cancer, muscle invasive urothelial carcinoma, colorectal cancer, metastatic colorectal cancer, fallopian tube cancer, gastroesophageal junction cancer (e.g., adenocarcinoma), larynx cancer (e.g., squamous cell), merkel cell cancer, mouth cancer, ovary cancer (e.g., epithelial), pancreas cancer (e.g., adenocarcinoma, metastatic), penis cancer (e.g., squamous cell carcinoma), peritoneum cancer, prostate cancer (e.g., castration-resistant, metastatic), rectum cancer, skin cancer (e.g., basal cell carcinoma, squamous cell carcinoma), small intestine cancer (e.g., adenocarcinoma), testicle cancer, thymus cancer, anaplastic thyroid cancer, cholangiocarcinoma, chordoma, cutaneous T-cell lymphoma, digestive-gastrointestinal cancer, familial pheochromocytoma-paraganglioma, Glioma, HTLV-1-associated adult T-cell leukemia-lymphoma, hematologic-blood cancer, hepatitis C (HCV), papillomaviral respiratory Infection, uterine leiomyosarcoma, acute lymphocytic leukemia, chronic myeloid leukemia, T-cell Lymphoma, follicular lymphoma, primary mediastinal large B-cell lymphoma, diffuse large B-cell testicular lymphoma, melanoma, malignant mesothelioma, pleural mesothelioma, mycosis fungoides, neuroendocrine cancer, oral epithelial dysplasia, Sarcoma, severe sepsis, sezary syndrome, smoldering myeloma, soft tissue sarcoma, nasal natural killer (NK) cell T-cell lymphoma, peripheral T-cell lymphoma.

In some embodiments, the cancer is selected from a list including but not limited to: breast cancer, ovarian carcinoma, acute myeloid leukemia, chronic myelogenous leukemia, Hodgkin's and Burkitt's lymphoma, diffuse large Bcell lymphoma, prostate cancer, colon cancer, gastric cancer, primary central nervous system lymphoma, glioblastoma, medulloblastoma, melanoma, non-small cell lung carcinoma, germinal center-derived lymphomas, esophageal squamous cell carcinoma, osteosarcoma, bladder cancer, pancreatic cancer, lung adenocarcinoma, BRAF V600E thyroid cancer, choroid plexus carcinoma, colitis-associated cancer, epithelial ovarian cancer, colorectal cancer, pancreatic cancer and uterine cancer.

In some embodiments, the cancer may be selected from solid tumors and non-solid tumors.

In various embodiments, this invention is directed to a method for suppressing, reducing or inhibiting tumor growth in a subject, comprising administering a compound of this invention, to a subject under conditions effective to suppress, reduce or inhibit tumor growth in said subject.

In some embodiments, the tumor may be a solid tumor or a non-solid tumor.

In some embodiments, the solid tumor cancer is selected from a list including but not limited to: breast cancer, ovarian carcinoma, prostate cancer, colon cancer, gastric cancer, glioblastoma, medulloblastoma, melanoma, non-small cell lung carcinoma, esophageal squamous cell carcinoma, osteosarcoma, bladder cancer, pancreatic cancer, lung adenocarcinoma, BRAF V600E thyroid cancer, choroid plexus carcinoma, colitis-associated cancer, epithelial ovarian cancer, colorectal cancer, pancreatic cancer and uterine cancer.

In some embodiments, the non-solid tumors include but not limited to: hematological malignancies including leukemia, lymphoma or myeloma and inherited cancers such as retinoblastoma and Wilm's tumor.

In some embodiments, the non-solid tumor cancer is selected from a list including but not limited to: acute myeloid leukemia, chronic myelogenous leukemia, Hodgkin's and Burkitt's lymphoma, diffuse large Bcell lymphoma, primary central nervous system lymphoma, glioblastoma, medulloblastoma, germinal center-derived lymphomas, myeloma, retinoblastoma and Wilm's tumor.

Therefore, and in various embodiments, this invention is directed to a method of treating, suppressing, reducing the severity, reducing the risk of developing or inhibiting cancer comprising administering a compound of this invention to a subject suffering from cancer under conditions effective to treat, suppress, reduce the severity, reduce the risk of developing, or inhibit the cancer. In some embodiments, the cancer is early cancer. In some embodiments, the cancer is advanced cancer. In some embodiments, the cancer is invasive cancer. In some embodiments, the cancer is metastatic cancer. In some embodiments, the cancer is drug resistant cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting breast cancer comprising administering a compound of this invention to a subject suffering from breast cancer under conditions effective to treat, suppress, reduce the severity, reduce the risk of developing, or inhibit the breast cancer. In some embodiments, the breast cancer is early breast cancer. In some embodiments, the breast cancer is advanced breast cancer. In some embodiments, the breast cancer is invasive breast cancer. In some embodiments, the breast cancer is metastatic breast cancer. In some embodiments, the breast cancer is drug resistant breast cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting ovarian carcinoma comprising administering a compound of this invention to a subject suffering from ovarian carcinoma under conditions effective to treat, suppress, reduce the severity, reduce the risk of developing, or inhibit the ovarian carcinoma. In some embodiments, the ovarian carcinoma is early ovarian carcinoma. In some embodiments, the ovarian carcinoma is advanced ovarian carcinoma. In some embodiments, the ovarian carcinoma is invasive ovarian carcinoma. In some embodiments, the ovarian carcinoma is metastatic ovarian carcinoma. In some embodiments, the ovarian carcinoma is drug resistant ovarian carcinoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting acute myeloid leukemia comprising administering a compound of this invention to a subject suffering from acute myeloid leukemia under conditions effective to treat, suppress, reduce the severity, reduce the risk of developing, or inhibit the acute myeloid leukemia. In some embodiments, the acute myeloid leukemia is early acute myeloid leukemia. In some embodiments, the acute myeloid leukemia is advanced acute myeloid leukemia. In some embodiments, the acute myeloid leukemia is invasive acute myeloid leukemia. In some embodiments, the acute myeloid leukemia is metastatic acute myeloid leukemia. In some embodiments, the acute myeloid leukemia is drug resistant acute myeloid leukemia. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting chronic myelogenous leukemia comprising administering a compound of this invention to a subject suffering from chronic myelogenous leukemia under conditions effective to treat, suppress, reduce the severity, reduce the risk of developing, or inhibit the chronic myelogenous leukemia. In some embodiments, the chronic myelogenous leukemia is early chronic myelogenous leukemia. In some embodiments, the chronic myelogenous leukemia is advanced chronic myelogenous leukemia. In some embodiments, the chronic myelogenous leukemia is invasive chronic myelogenous leukemia. In some embodiments, the chronic myelogenous leukemia is metastatic chronic myelogenous leukemia. In some embodiments, the chronic myelogenous leukemia is drug resistant chronic myelogenous leukemia. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting Hodgkin's and/or Burkitt's lymphoma comprising administering a compound of this invention to a subject suffering from Hodgkin's and/or Burkitt's lymphoma under conditions effective to treat, suppress, reduce the severity, reduce the risk of developing, or inhibit the Hodgkin's and/or Burkitt's lymphoma. In some embodiments, the Hodgkin's and/or Burkitt's lymphoma is early Hodgkin's and/or Burkitt's lymphoma. In some embodiments, the Hodgkin's and/or Burkitt's lymphoma is advanced Hodgkin's and/or Burkitt's lymphoma. In some embodiments, the Hodgkin's and/or Burkitt's lymphoma is invasive Hodgkin's and/or Burkitt's lymphoma. In some embodiments, the Hodgkin's and/or Burkitt's lymphoma is metastatic Hodgkin's and/or Burkitt's lymphoma. In some embodiments, the Hodgkin's and/or Burkitt's lymphoma is drug resistant Hodgkin's and/or Burkitt's lymphoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting diffuse large Bcell lymphoma comprising administering a compound of this invention to a subject suffering from diffuse large Bcell lymphoma under conditions effective to treat, suppress, reduce the severity, reduce the risk of developing, or inhibit the diffuse large Bcell lymphoma. In some embodiments, the diffuse large Bcell lymphoma is early diffuse large Bcell lymphoma. In some embodiments, the diffuse large Bcell lymphoma is advanced diffuse large Bcell lymphoma. In some embodiments, the diffuse large Bcell lymphoma is invasive diffuse large Bcell lymphoma. In some embodiments, the diffuse large Bcell lymphoma is metastatic diffuse large Bcell lymphoma. In some embodiments, the diffuse large Bcell lymphoma is drug resistant diffuse large Bcell lymphoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting prostate cancer comprising administering a compound of this invention to a subject suffering from prostate cancer under conditions effective to treat, suppress, reduce the severity, reduce the risk of developing, or inhibit the prostate cancer. In some embodiments, the prostate cancer is early prostate cancer. In some embodiments, the prostate cancer is advanced prostate cancer. In some embodiments, the prostate cancer is invasive prostate cancer. In some embodiments, the prostate cancer is metastatic prostate cancer. In some embodiments, the prostate cancer is drug resistant prostate cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting colon cancer comprising administering a compound of this invention to a subject suffering from colon cancer under conditions effective to treat, suppress, reduce the severity, reduce the risk of developing, or inhibit the colon cancer. In some embodiments, the colon cancer is early colon cancer. In some embodiments, the colon cancer is advanced colon cancer. In some embodiments, the colon cancer is invasive colon cancer. In some embodiments, the colon cancer is metastatic colon cancer. In some embodiments, the colon cancer is drug resistant colon cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting gastric cancer comprising administering a compound of this invention to a subject suffering from gastric cancer under conditions effective to treat, suppress, reduce the severity, reduce the risk of developing, or inhibit the gastric cancer. In some embodiments, the gastric cancer is early gastric cancer. In some embodiments, the gastric cancer is advanced gastric cancer. In some embodiments, the gastric cancer is invasive gastric cancer. In some embodiments, the gastric cancer is metastatic gastric cancer. In some embodiments, the gastric cancer is drug resistant gastric cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting lymphoma comprising administering a compound of this invention to a subject suffering from lymphoma under conditions effective to treat, suppress, reduce the severity, reduce the risk of developing, or inhibit the lymphoma. In some embodiments, the lymphoma is early lymphoma. In some embodiments, the lymphoma is advanced lymphoma. In some embodiments, the lymphoma is invasive lymphoma. In some embodiments, the lymphoma is metastatic lymphoma. In some embodiments, the lymphoma is drug resistant lymphoma. In some embodiments, the lymphoma is primary central nervous system lymphoma. In some embodiments, the lymphoma is germinal center-derived lymphoma. In some embodiments, the lymphoma is Hodgkin's lymphoma. In some embodiments, the lymphoma is Burkitt's lymphoma. In some embodiments, the lymphoma is diffuse large B-cell lymphoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting glioblastoma comprising administering a compound of this invention to a subject suffering from glioblastoma under conditions effective to treat, suppress, reduce the severity, reduce the risk of developing, or inhibit the glioblastoma. In some embodiments, the glioblastoma is early glioblastoma. In some embodiments, the glioblastoma is advanced glioblastoma. In some embodiments, the glioblastoma is invasive glioblastoma. In some embodiments, the glioblastoma is metastatic glioblastoma. In some embodiments, the glioblastoma is drug resistant glioblastoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting medulloblastoma comprising administering a compound of this invention to a subject suffering from medulloblastoma under conditions effective to treat, suppress, reduce the severity, reduce the risk of developing, or inhibit the medulloblastoma. In some embodiments, the medulloblastoma is early medulloblastoma. In some embodiments, the medulloblastoma is advanced medulloblastoma. In some embodiments, the medulloblastoma is invasive medulloblastoma. In some embodiments, the medulloblastoma is metastatic medulloblastoma. In some embodiments, the medulloblastoma is drug resistant medulloblastoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting melanoma comprising administering a compound of this invention to a subject suffering from melanoma under conditions effective to treat, suppress, reduce the severity, reduce the risk of developing, or inhibit the melanoma. In some embodiments, the melanoma is early melanoma. In some embodiments, the melanoma is advanced melanoma. In some embodiments, the melanoma is invasive melanoma. In some embodiments, the melanoma is metastatic melanoma. In some embodiments, the melanoma is drug resistant melanoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting non-small cell lung carcinoma comprising administering a compound of this invention to a subject suffering from non-small cell lung carcinoma under conditions effective to treat, suppress, reduce the severity, reduce the risk of developing, or inhibit the non-small cell lung carcinoma. In some embodiments, the non-small cell lung carcinoma is early non-small cell lung carcinoma. In some embodiments, the non-small cell lung carcinoma is advanced non-small cell lung carcinoma. In some embodiments, the non-small cell lung carcinoma is invasive non-small cell lung carcinoma. In some embodiments, the non-small cell lung carcinoma is metastatic non-small cell lung carcinoma. In some embodiments, the non-small cell lung carcinoma is drug resistant non-small cell lung carcinoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting esophageal squamous cell carcinoma comprising administering a compound of this invention to a subject suffering from esophageal squamous cell carcinoma under conditions effective to treat, suppress, reduce the severity, reduce the risk of developing, or inhibit the esophageal squamous cell carcinoma. In some embodiments, the esophageal squamous cell carcinoma is early esophageal squamous cell carcinoma. In some embodiments, the esophageal squamous cell carcinoma is advanced esophageal squamous cell carcinoma. In some embodiments, the esophageal squamous cell carcinoma is invasive esophageal squamous cell carcinoma. In some embodiments, the esophageal squamous cell carcinoma is metastatic esophageal squamous cell carcinoma. In some embodiments, the esophageal squamous cell carcinoma is drug resistant esophageal squamous cell carcinoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting osteosarcoma comprising administering a compound of this invention to a subject suffering from osteosarcoma under conditions effective to treat, suppress, reduce the severity, reduce the risk of developing, or inhibit the osteosarcoma. In some embodiments, the osteosarcoma is early osteosarcoma. In some embodiments, the osteosarcoma is advanced osteosarcoma. In some embodiments, the osteosarcoma is invasive osteosarcoma. In some embodiments, the osteosarcoma is metastatic osteosarcoma. In some embodiments, the osteosarcoma is drug resistant osteosarcoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting bladder cancer comprising administering a compound of this invention to a subject suffering from bladder cancer under conditions effective to treat, suppress, reduce the severity, reduce the risk of developing, or inhibit the bladder cancer. In some embodiments, the bladder cancer is early bladder cancer. In some embodiments, the bladder cancer is advanced bladder cancer. In some embodiments, the bladder cancer is invasive bladder cancer. In some embodiments, the bladder cancer is metastatic bladder cancer. In some embodiments, the bladder cancer is drug resistant bladder cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting pancreatic cancer comprising administering a compound of this invention to a subject suffering from pancreatic cancer under conditions effective to treat, suppress, reduce the severity, reduce the risk of developing, or inhibit the pancreatic cancer. In some embodiments, the pancreatic cancer is early pancreatic cancer. In some embodiments, the pancreatic cancer is advanced pancreatic cancer. In some embodiments, the pancreatic cancer is invasive pancreatic cancer. In some embodiments, the pancreatic cancer is metastatic pancreatic cancer. In some embodiments, the pancreatic cancer is drug resistant pancreatic cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting lung adenocarcinoma comprising administering a compound of this invention to a subject suffering from lung adenocarcinoma under conditions effective to treat, suppress, reduce the severity, reduce the risk of developing, or inhibit the lung adenocarcinoma. In some embodiments, the lung adenocarcinoma is early lung adenocarcinoma. In some embodiments, the lung adenocarcinoma is advanced lung adenocarcinoma. In some embodiments, the lung adenocarcinoma is invasive lung adenocarcinoma. In some embodiments, the lung adenocarcinoma is metastatic lung adenocarcinoma. In some embodiments, the lung adenocarcinoma is drug resistant lung adenocarcinoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting thyroid cancer comprising administering a compound of this invention to a subject suffering from thyroid cancer under conditions effective to treat, suppress, reduce the severity, reduce the risk of developing, or inhibit the thyroid cancer. In some embodiments, the thyroid cancer is early thyroid cancer. In some embodiments, the thyroid cancer is advanced thyroid cancer. In some embodiments, the thyroid cancer is invasive thyroid cancer. In some embodiments, the thyroid cancer is metastatic thyroid cancer. In some embodiments, the thyroid cancer is drug resistant thyroid cancer. In some embodiments, the thyroid cancer is BRAF V600E thyroid cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting choroid plexus carcinoma comprising administering a compound of this invention to a subject suffering from choroid plexus carcinoma under conditions effective to treat, suppress, reduce the severity, reduce the risk of developing, or inhibit the choroid plexus carcinoma. In some embodiments, the choroid plexus carcinoma is early choroid plexus carcinoma. In some embodiments, the choroid plexus carcinoma is advanced choroid plexus carcinoma. In some embodiments, the choroid plexus carcinoma is invasive choroid plexus carcinoma. In some embodiments, the choroid plexus carcinoma is metastatic choroid plexus carcinoma. In some embodiments, the choroid plexus carcinoma is drug resistant choroid plexus carcinoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting colitis-associated cancer comprising administering a compound of this invention to a subject suffering from colitis-associated cancer under conditions effective to treat, suppress, reduce the severity, reduce the risk of developing, or inhibit the colitis-associated cancer. In some embodiments, the colitis-associated cancer is early colitis-associated cancer. In some embodiments, the colitis-associated cancer is advanced colitis-associated cancer. In some embodiments, the colitis-associated cancer is invasive colitis-associated cancer. In some embodiments, the colitis-associated cancer is metastatic colitis-associated cancer. In some embodiments, the colitis-associated cancer is drug resistant colitis-associated cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting ovarian cancer comprising administering a compound of this invention to a subject suffering from ovarian cancer under conditions effective to treat, suppress, reduce the severity, reduce the risk of developing, or inhibit the ovarian cancer. In some embodiments, the ovarian cancer is early ovarian cancer. In some embodiments, the ovarian cancer is advanced ovarian cancer. In some embodiments, the ovarian cancer is invasive ovarian cancer. In some embodiments, the ovarian cancer is metastatic ovarian cancer. In some embodiments, the ovarian cancer is drug resistant ovarian cancer. In some embodiments, the ovarian cancer is epithelial ovarian cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting colorectal cancer comprising administering a compound of this invention to a subject suffering from colorectal cancer under conditions effective to treat, suppress, reduce the severity, reduce the risk of developing, or inhibit the colorectal cancer. In some embodiments, the colorectal cancer is early colorectal cancer. In some embodiments, the colorectal cancer is advanced colorectal cancer. In some embodiments, the colorectal cancer is invasive colorectal cancer. In some embodiments, the colorectal cancer is metastatic colorectal cancer. In some embodiments, the colorectal cancer is drug resistant colorectal cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting uterine cancer comprising administering a compound of this invention to a subject suffering from uterine cancer under conditions effective to treat, suppress, reduce the severity, reduce the risk of developing, or inhibit the uterine cancer. In some embodiments, the uterine cancer is early uterine cancer. In some embodiments, the uterine cancer is advanced uterine cancer. In some embodiments, the uterine cancer is invasive uterine cancer. In some embodiments, the uterine cancer is metastatic uterine cancer. In some embodiments, the uterine cancer is drug resistant uterine cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention provides methods for increasing the survival of a subject suffering from metastatic cancer comprising the step of administering to said subject a compound of this invention and/or an isomer, metabolite, pharmaceutically acceptable salt, pharmaceutical product, tautomer, hydrate, N-oxide, reverse amide analog, prodrug, isotopic variant (e.g., deuterated analog), PROTAC, polymorph, or crystal of said compound, or any combination thereof. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the cancer is breast cancer, ovarian carcinoma, acute myeloid leukemia, chronic myelogenous leukemia, Hodgkin's and Burkitt's lymphoma, diffuse large Bcell lymphoma, prostate cancer, colon cancer, gastric cancer, primary central nervous system lymphoma, glioblastoma, medulloblastoma, melanoma, non-small cell lung carcinoma, germinal center-derived lymphomas, esophageal squamous cell carcinoma, osteosarcoma, bladder cancer, pancreatic cancer, lung adenocarcinoma, thyroid cancer, choroid plexus carcinoma, colitis-associated cancer, colorectal cancer, or uterine cancer; each represents a separate embodiment according to this invention.

In various embodiments, this invention provides methods for treating, suppressing, reducing the severity, reducing the risk, or inhibiting advanced cancer comprising the step of administering to said subject a compound of this invention and/or an isomer, metabolite, pharmaceutically acceptable salt, pharmaceutical product, tautomer, hydrate, N-oxide, reverse amide analog, prodrug, isotopic variant (e.g., deuterated analog), PROTAC, polymorph, or crystal of said compound, or any combination thereof. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the cancer is breast cancer, ovarian carcinoma, acute myeloid leukemia, chronic myelogenous leukemia, Hodgkin's and Burkitt's lymphoma, diffuse large Bcell lymphoma, prostate cancer, colon cancer, gastric cancer, primary central nervous system lymphoma, glioblastoma, medulloblastoma, melanoma, non-small cell lung carcinoma, germinal center-derived lymphomas, esophageal squamous cell carcinoma, osteosarcoma, bladder cancer, pancreatic cancer, lung adenocarcinoma, thyroid cancer, choroid plexus carcinoma, colitis-associated cancer, colorectal cancer, or uterine cancer; each represents a separate embodiment according to this invention.

The compounds of the present invention are useful in the treatment, reducing the severity, reducing the risk of developing, or inhibition of early cancer, metastatic cancer, advanced cancer, drug resistant cancer, and various forms of cancer. In a preferred embodiment the cancer is breast cancer, ovarian carcinoma, acute myeloid leukemia, chronic myelogenous leukemia, Hodgkin's and Burkitt's lymphoma, diffuse large Bcell lymphoma, prostate cancer, colon cancer, gastric cancer, primary central nervous system lymphoma, glioblastoma, medulloblastoma, melanoma, non-small cell lung carcinoma, germinal center-derived lymphomas, esophageal squamous cell carcinoma, osteosarcoma, bladder cancer, pancreatic cancer, lung adenocarcinoma, thyroid cancer, choroid plexus carcinoma, colitis-associated cancer, colorectal cancer, or uterine cancer; each represents a separate embodiment according to this invention. Based upon their believed mode of action, it is believed that other forms of cancer will likewise be treatable or preventable upon administration of the compounds or compositions of the present invention to a patient. Preferred compounds of the present invention are selectively disruptive to cancer cells, causing ablation of cancer cells but preferably not normal cells. Significantly, harm to normal cells is minimized because the cancer cells are susceptible to disruption at much lower concentrations of the compounds of the present invention.

In various embodiments, other types of cancers that may be treatable with the c-MYC mRNA translation modulators according to this invention include: adrenocortical carcinoma, anal cancer, bladder cancer, brain tumor, brain stem tumor, breast cancer, glioma, cerebellar astrocytoma, cerebral astrocytoma, ependymoma, medulloblastoma, supratentorial primitive neuroectodermal, pineal tumors, hypothalamic glioma, carcinoid tumor, carcinoma, cervical cancer, colon cancer, central nervous system (CNS) cancer, endometrial cancer, esophageal cancer, extrahepatic bile duct cancer, Ewing's family of tumors (Pnet), extracranial germ cell tumor, eye cancer, intraocular melanoma, gallbladder cancer, gastric cancer, germ cell tumor, extragonadal, gestational trophoblastic tumor, head and neck cancer, hypopharyngeal cancer, islet cell carcinoma, laryngeal cancer, leukemia, acute lymphoblastic, leukemia, oral cavity cancer, liver cancer, lung cancer, non-small cell lung cancer, small cell, lymphoma, AIDS-related lymphoma, central nervous system (primary), lymphoma, cutaneous T-cell, lymphoma, Hodgkin's disease, non-Hodgkin's disease, malignant mesothelioma, melanoma, Merkel cell carcinoma, metasatic squamous carcinoma, multiple myeloma, mycosis fungoides, myelodysplastic syndrome, myeloproliferative disorders, nasopharyngeal cancer, neuroblastoma, oropharyngeal cancer, osteosarcoma, ovarian cancer, ovarian epithelial cancer, ovarian germ cell tumor, ovarian low malignant potential tumor, pancreatic cancer, exocrine, pancreatic cancer, islet cell carcinoma, paranasal sinus and nasal cavity cancer, parathyroid cancer, penile cancer, pheochromocytoma cancer, pituitary cancer, plasma cell neoplasm, prostate cancer, rhabdomyosarcoma, rectal cancer, renal cancer, renal cell cancer, salivary gland cancer, Sezary syndrome, skin cancer, cutaneous T-cell lymphoma, Kaposi's sarcoma, melanoma, small intestine cancer, soft tissue sarcoma, testicular cancer, thymoma, malignant, thyroid cancer, urethral cancer, uterine cancer, sarcoma, unusual cancer of childhood, vaginal cancer, vulvar cancer, Wilms' tumor, hepatocellular cancer, hematological cancer or any combination thereof. In some embodiments the cancer is invasive. In some embodiments the cancer is metastatic cancer. In some embodiments the cancer is advanced cancer. In some embodiments the cancer is drug resistant cancer.

In various embodiments “metastatic cancer” refers to a cancer that spread (metastasized) from its original site to another area of the body. Virtually all cancers have the potential to spread. Whether metastases develop depends on the complex interaction of many tumor cell factors, including the type of cancer, the degree of maturity (differentiation) of the tumor cells, the location and how long the cancer has been present, as well as other incompletely understood factors. Metastases spread in three ways—by local extension from the tumor to the surrounding tissues, through the bloodstream to distant sites or through the lymphatic system to neighboring or distant lymph nodes. Each kind of cancer may have a typical route of spread. The tumor is called by the primary site (ex. breast cancer that has spread to the brain is called metastatic breast cancer to the brain).

In various embodiments “drug-resistant cancer” refers to cancer cells that acquire resistance to chemotherapy. Cancer cells can acquire resistance to chemotherapy by a range of mechanisms, including the mutation or overexpression of the drug target, inactivation of the drug, or elimination of the drug from the cell. Tumors that recur after an initial response to chemotherapy may be resistant to multiple drugs (they are multidrug resistant). In the conventional view of drug resistance, one or several cells in the tumor population acquire genetic changes that confer drug resistance. Accordingly, the reasons for drug resistance, inter alia, are: a) some of the cells that are not killed by the chemotherapy mutate (change) and become resistant to the drug. Once they multiply, there may be more resistant cells than cells that are sensitive to the chemotherapy; b) Gene amplification. A cancer cell may produce hundreds of copies of a particular gene. This gene triggers an overproduction of protein that renders the anticancer drug ineffective; c) cancer cells may pump the drug out of the cell as fast as it is going in using a molecule called p-glycoprotein; d) cancer cells may stop taking in the drugs because the protein that transports the drug across the cell wall stops working; e) the cancer cells may learn how to repair the DNA breaks caused by some anti-cancer drugs; f) cancer cells may develop a mechanism that inactivates the drug. One major contributor to multidrug resistance is overexpression of P-glycoprotein (P-gp). This protein is a clinically important transporter protein belonging to the ATP-binding cassette family of cell membrane transporters. It can pump substrates including anticancer drugs out of tumor cells through an ATP-dependent mechanism; g) Cells and tumors with activating RAS mutations are relatively resistant to most anti-cancer agents. Thus, the resistance to anticancer agents used in chemotherapy is the main cause of treatment failure in malignant disorders, provoking tumors to become resistant. Drug resistance is the major cause of cancer chemotherapy failure.

In various embodiments “resistant cancer” refers to drug-resistant cancer as described herein above. In some embodiments “resistant cancer” refers to cancer cells that acquire resistance to any treatment such as chemotherapy, radiotherapy or biological therapy.

In various embodiments, this invention is directed to treating, suppressing, reducing the severity, reducing the risk of developing, or inhibiting cancer in a subject, wherein the subject has been previously treated with chemotherapy, radiotherapy or biological therapy.

In various embodiments “Chemotherapy” refers to chemical treatment for cancer such as drugs that kill cancer cells directly. Such drugs are referred as “anti-cancer” drugs or “antineoplastics.” Today's therapy uses more than 100 drugs to treat cancer. Chemotherapy is used to control tumor growth when cure is not possible; to shrink tumors before surgery or radiation therapy; to relieve symptoms (such as pain); and to destroy microscopic cancer cells that may be present after the known tumor is removed by surgery (called adjuvant therapy). Adjuvant therapy is given to prevent a possible cancer reoccurrence.

In various embodiments, “Radiotherapy” (also referred herein as “Radiation therapy”) refers to high energy x-rays and similar rays (such as electrons) to treat disease. Many people with cancer will have radiotherapy as part of their treatment. This can be given either as external radiotherapy from outside the body using x-rays or from within the body as internal radiotherapy. Radiotherapy works by destroying the cancer cells in the treated area. Although normal cells can also be damaged by the radiotherapy, they can usually repair themselves. Radiotherapy treatment can cure some cancers and can also reduce the chance of a cancer coming back after surgery. It may be used to reduce cancer symptoms.

In various embodiments “Biological therapy” refers to substances that occur naturally in the body to destroy cancer cells. There are several types of treatment including: monoclonal antibodies, cancer growth inhibitors, vaccines and gene therapy. Biological therapy is also known as immunotherapy.

When the compounds or pharmaceutical compositions of the present invention are administered to treat, suppress, reduce the severity, reduce the risk of developing, or inhibit a cancerous condition, the pharmaceutical composition can also contain, or can be administered in conjunction with, other therapeutic agents or treatment regimen presently known or hereafter developed for the treatment of various types of cancer. Examples of other therapeutic agents or treatment regimen include, without limitation, radiation therapy, immunotherapy, chemotherapy, surgical intervention, and combinations thereof.

In various embodiments, the compound according to this invention, is administered in combination with an anti-cancer therapy. Examples of such therapies include but are not limited to: chemotherapy, immunotherapy, radiotherapy, biological therapy, surgical intervention, and combinations thereof.

In various embodiments, the compound is administered in combination with an anti-cancer agent by administering the compounds as herein described, alone or in combination with other agents.

In various embodiments, the composition for cancer treatment of the present invention can be used together with existing chemotherapy drugs or be made as a mixture with them. Such a chemotherapy drug includes, for example, alkylating agents, nitrosourea agents, antimetabolites, antitumor antibiotics, alkaloids derived from plant, topoisomerase inhibitors, hormone therapy medicines, hormone antagonists, aromatase inhibitors, P-glycoprotein inhibitors, platinum complex derivatives, other immunotherapeutic drugs, and other anticancer agents. Further, they can be used together with hypoleukocytosis (neutrophil) medicines that are cancer treatment adjuvant, thrombopenia medicines, antiemetic drugs, and cancer pain medicines for patient's QOL recovery or be made as a mixture with them.

In various embodiments, this invention provides a method of modulating c-MYC mRNA translation in a cell, comprising contacting a compound represented by the structure of formula I and/or I(a)-I(n) and/or by the structures listed in Table 1, as defined herein above, with a cell, thereby modulating c-MYC mRNA translation in said cell. In some embodiments, the method is carried out by regulating c-MYC mRNA splicing. In some embodiments, the method is carried out by inclusion or exclusion of untranslated region or alternative usage of exons. In some embodiments, the method is carried out by regulation of c-MYC mRNA modifications. In some embodiments, the method is carried out by regulation of the interaction of RNA binding protein with c-MYC mRNA thereby changing mRNA localization. In some embodiments, the method is carried out by regulating c-MYC mRNA localization in the cytoplasm. In some embodiments, the method is carried out by regulating ribosomes or ribosome accessory factor to c-MYC mRNA. In some embodiments, the method is carried out by reducing the amount of c-MYC protein in the cell.

This invention further provides a method of regulating c-MYC mRNA transcription in a cell, comprising contacting a compound represented by the structure of formula I and/or I(a)-I(n) and/or by the structures listed in Table 1, as defined herein above, with a cell, thereby regulating c-MYC mRNA transcription in said cell. In some embodiments, the method is carried out by regulating c-MYC mRNA splicing. In some embodiments, the method is carried out by inclusion or exclusion of untranslated region or alternative usage of exons. In some embodiments, the method is carried out by regulation of c-MYC mRNA modifications. In some embodiments, the method is carried out by regulation of the interaction of RNA binding protein with c-MYC mRNA thereby changing mRNA localization. In some embodiments, the method is carried out by regulating c-MYC mRNA localization in the cytoplasm. In some embodiments, the method is carried out by regulating ribosomes or ribosome accessory factor to c-MYC mRNA. In some embodiments, the method is carried out by reducing the amount of c-MYC protein in the cell.

In various embodiments, this invention is directed to a method of destroying a cancerous cell comprising providing a compound of this invention and contacting the cancerous cell with the compound under conditions effective to destroy the contacted cancerous cell. According to various embodiments of destroying the cancerous cells, the cells to be destroyed can be located either in vivo or ex vivo (i.e., in culture).

A still further aspect of the present invention relates to a method of treating or preventing a cancerous condition that includes providing a compound of the present invention and then administering an effective amount of the compound to a patient in a manner effective to treat or prevent a cancerous condition.

According to one embodiment, the patient to be treated is characterized by the presence of a precancerous condition, and the administering of the compound is effective to prevent development of the precancerous condition into the cancerous condition. This can occur by destroying the precancerous cell prior to or concurrent with its further development into a cancerous state.

According to other embodiments, the patient to be treated is characterized by the presence of a cancerous condition, and the administering of the compound is effective either to cause regression of the cancerous condition or to inhibit growth of the cancerous condition, i.e., stopping its growth altogether or reducing its rate of growth. This preferably occurs by destroying cancer cells, regardless of their location in the patient body. That is, whether the cancer cells are located at a primary tumor site or whether the cancer cells have metastasized and created secondary tumors within the patient body.

As used herein, subject or patient refers to any mammalian patient, including without limitation, humans and other primates, dogs, cats, horses, cows, sheep, pigs, rats, mice, and other rodents. In various embodiments, the subject is male. In some embodiments, the subject is female. In some embodiments, while the methods as described herein may be useful for treating either males or females.

When administering the compounds of the present invention, they can be administered systemically or, alternatively, they can be administered directly to a specific site where cancer cells or precancerous cells are present. Thus, administering can be accomplished in any manner effective for delivering the compounds or the pharmaceutical compositions to the cancer cells or precancerous cells. Exemplary modes of administration include, without limitation, administering the compounds or compositions orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes.

The following examples are presented in order to more fully illustrate the preferred embodiments of the invention. They should in no way, however, be construed as limiting the broad scope of the invention.

### EXAMPLES

### Example 1

### General Synthetic Details for Compounds of the Invention (Schemes 1-9)

### General Methods

All reagents were commercial grade and were used as received without further purification, unless otherwise specified. Reagent grade solvents were used in all cases, unless otherwise specified. Thin layer chromatography was carried out using pre-coated silica gel F-254 plates (thickness 0.25 mm). ¹H-NMR and ¹⁹F-NMR spectra were recorded on a Bruker Bruker Avance 400 MHz or Avance III 400 MHz spectrometer. The chemical shifts are expressed in ppm using the residual solvent as internal standard. Splitting patterns are designated as s (singlet), d (doublet), dd (doublet of doublets), t (triplet), dt (doublet of triplets), q (quartet), m (multiplet) and br s (broad singlet).

### Abbreviations

AcOH Acetic acid
amphos Bis(di-tert-butyl(4-dimethylaminophenyl)phosphine
Boc tert-Butyloxycarbonyl
BuLi n-butyllithium
t-BuLi tert-butyllithium
DBU 1,8-Diazabicyclo[5.4.0]undec-7-ene
dppb 1,4-Bis(diphenylphosphino)butane
dppf 1,1′-Bis(diphenylphosphino)ferrocene
DCM Dichloromethane
DCE 1,2-Dichloroethane
DIBAL-H Diisobutylaluminum hydride
DIPEA N,N-Diisopropylethylamine
DMF N,N-Dimethylformamide
DMA N,N-Dimethylacetamide
DMAP 4-Dimethylaminopyridine
DME 1,2-Dimethoxyethane
DMSO Dimethylsulfoxide
DPPA Diphenyl phosphoryl azide
DTBF 1,1′-Bis(di-tert-butylphosphino)ferrocene
EDC·HCl N-(3-Dimethylaminopropyl)-N-ethylcarbodiimide hydrochloride
HATU [O-(7-Azabenzotriazol-1-yl)-N,N,N′,N′-tetramethyluronium-hexafluorphosphate]
HPLC High performance liquid chromatography
MsCl Methanesulfonyl chloride
NBS N-Bromosuccinimide
POBr₃ Phosphorus(V) oxybromide
Py-HBr₃ Pyridinium tribromide
SEM 2-(Trimethylsilyl)ethoxymethyl
T3P Propylphosphonic anhydride
TBAF Tetrabutylammonium fluoride
TCFH N,N,N′,N′-tetramethylchloroformamidinium hexafluorophosphate
TFA Trifluoroacetic acid
THF Tetrahydrofuran
TMS-OTf Trimethylsilyl trifluoromethanesulfonate

### General Synthesis of Compounds of the Invention

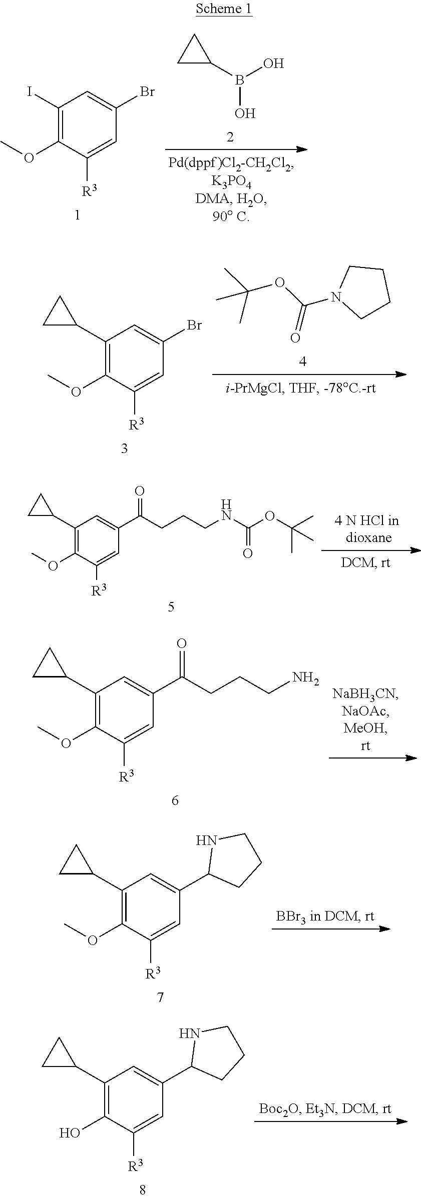

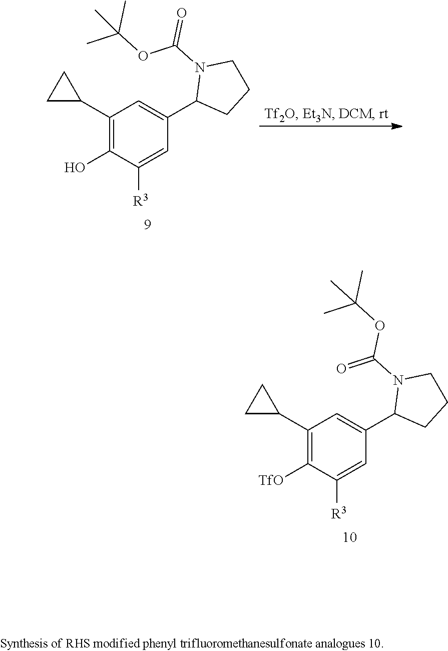

The first step of the synthesis involved a Suzuki coupling reaction between poly-substituted iodobenzenes 1 and cyclopropylboronic acid 2 to afford intermediates 3. Halogen magnesium exchange of intermediates 3 by isopropylmagnesium bromide at low temperature formed new aryl magnesium reagents, which were treated with tert-butyl 2-oxopyrrolidine-1-carboxylate 4 to generate aryl alkyl ketones 5. Subsequently the N-Boc group was removed under acidic conditions to give amine intermediates 6 as the corresponding hydrochloride salts or free bases. Intermediates 6 were subjected to an intramolecular reductive amination reaction to generate intermediates 7. The methoxy protecting groups of aryl methyl ether intermediates 7 were removed using boron tribromide to give amino poly-substituted phenols 8. Intermediates 8 were subjected to amino group protection with Boc groups to generate intermediates 9. In the final step the hydroxyl groups of intermediates 9 were converted to the corresponding trifluoromethanesulfonates by treatment with trifluoromethanesulfonic anhydride under basic conditions to give elaborated intermediates 10.

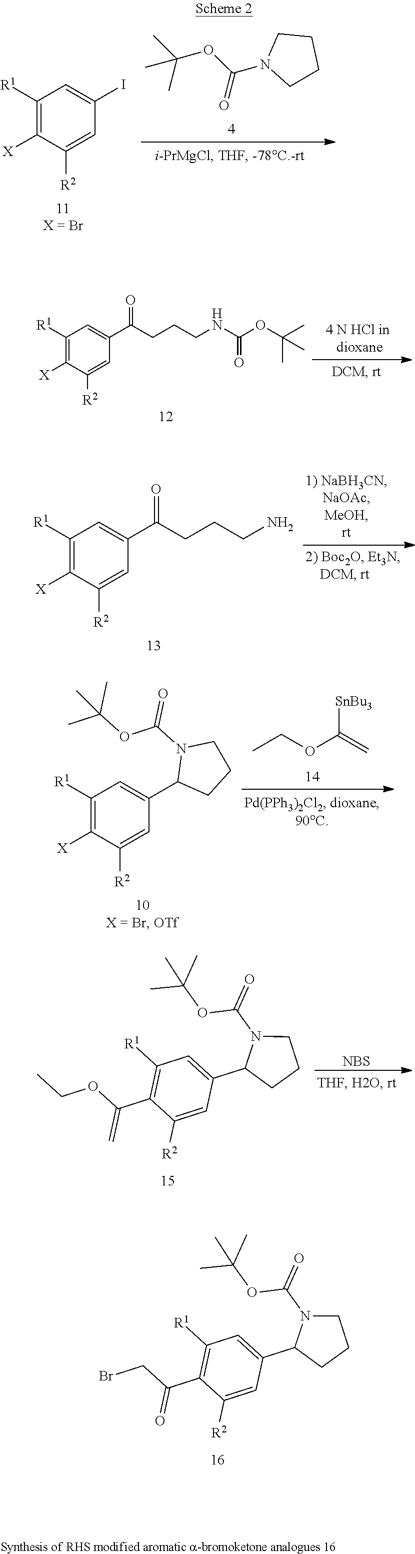

The first 3 steps were conducted using similar methodology the corresponding steps described in Scheme 1 to generate intermediates 10. Subsequently a 1-ethoxyvinyl group was introduced via Stille coupling of intermediates 10 with tributyl(1-ethoxyvinyl)stannane 14 affording intermediates 15. Intermediates 15 were treated with NBS in THF and water to give the corresponding aromatic a-bromoketones 16.

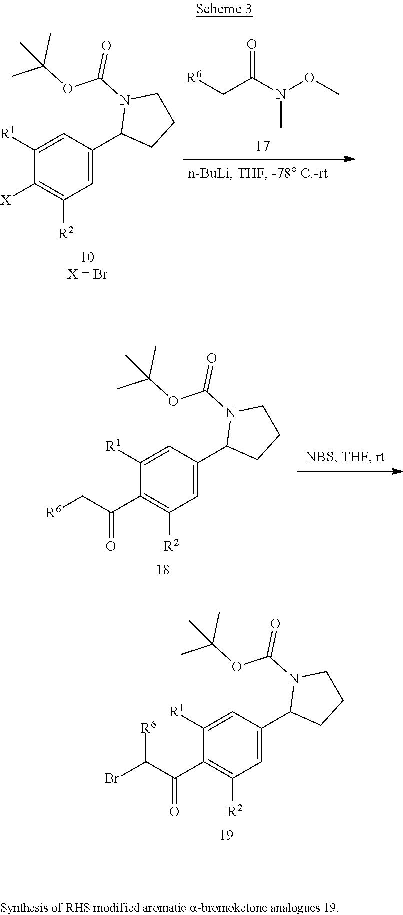

The corresponding step involved lithium halogen exchange of intermediates 10 with n-butyl lithium, followed by quenching with substituted Weinreb amides 17 forming a-substituted aromatic ketone intermediates 18. Subsequent bromination at a-position of ketone intermediates 18 with brominating reagents such as NBS generated a-bromoketone intermediates 19.

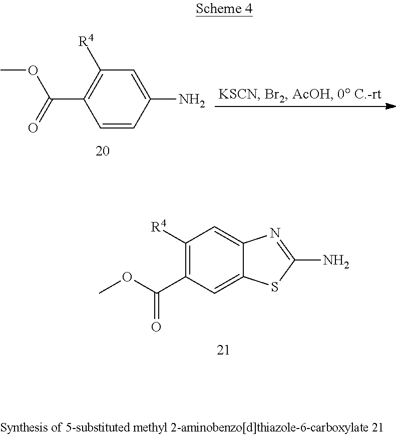

Intermediates 20 were treated with potassium thiocyanate and bromine in acetic acid to generate corresponding 2-aminobenzo[d]thiazoles 21.

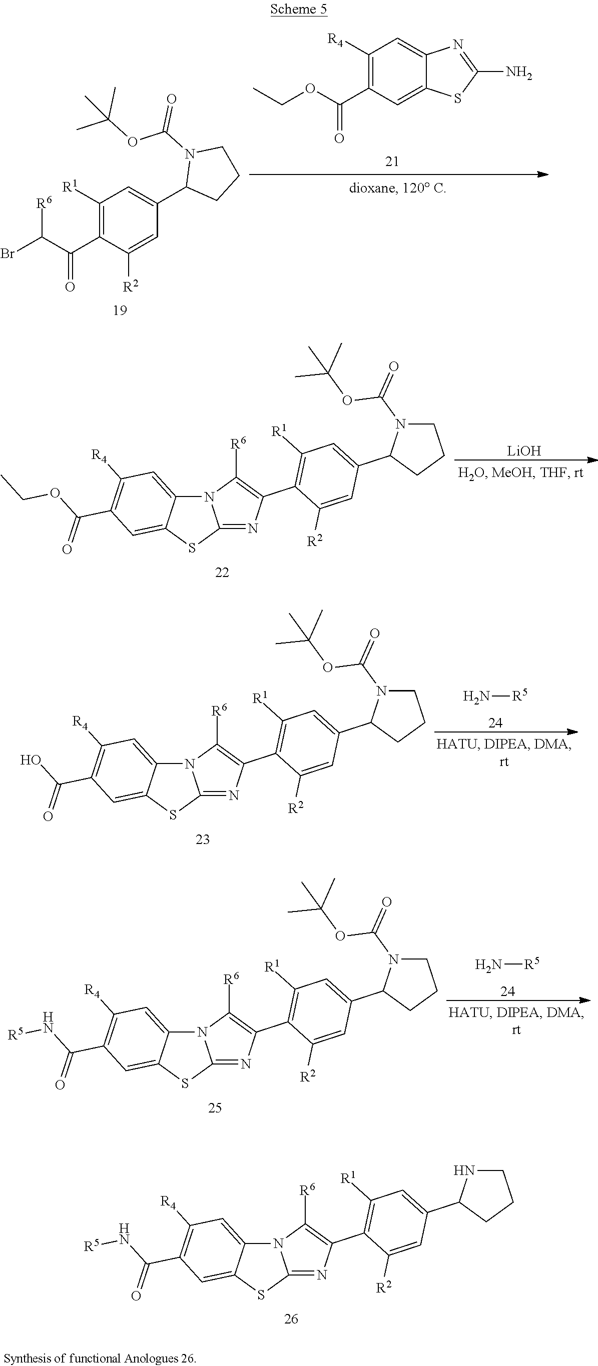

The initial step involved a cyclization reaction between modified a-bromoketone analogues 19 and corresponding 2-aminobenzo[d]thiazole 21 affording substituted tricyclic benzo[d]imidazo[2,1-b]thiazole intermediates 22. The carboxylic esters 22 were hydrolyzed under basic conditions such as aqueous lithium hydroxide to afford carboxylic acids 23. Subsequent condensation reaction of intermediates 23 with corresponding amines afforded N-Boc protected amides 25. Intermediates 25 were deprotected under acidic conditions such as hydrogen chloride in dioxane solution to generate final compounds 26.

### Synthesis of benzo[d]imidazo[2,1-b]thiazole Compounds, Structure I

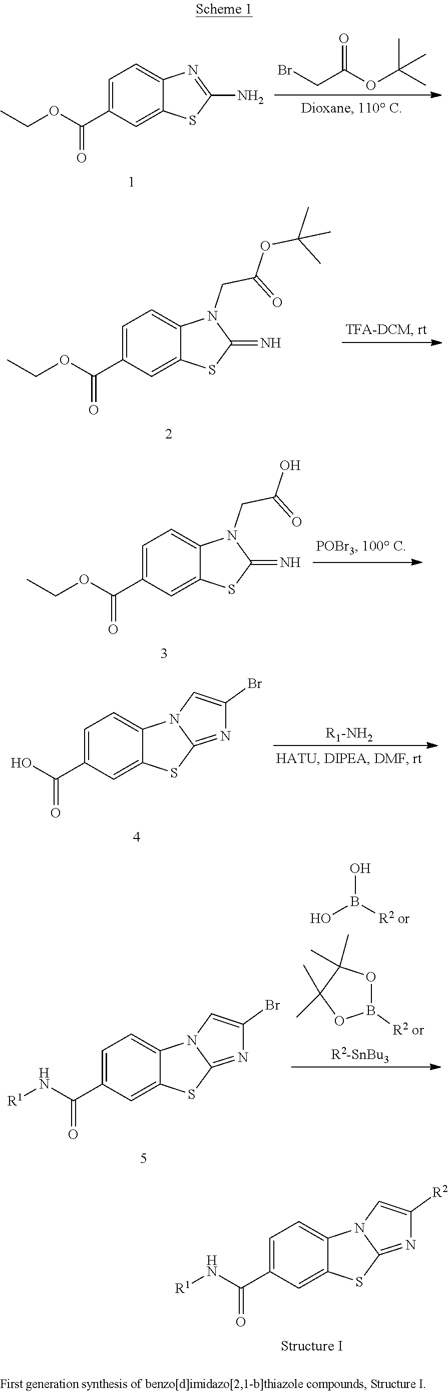

The first step of the synthesis involves alkylation of ethyl 2-aminobenzothiazole-6-carboxylate 1 (Scheme 6) with tert-butyl bromoacetate at elevated temperature affording alkylated intermediate 2 (Scheme 6). The tert-butyl group was removed using a mixture of TFA-DCM to generate the carboxylic acid intermediate 3. Treatment of the carboxylic acid intermediate 3 (Scheme 6) with phosphorus(V) oxybromide at elevated temperature results in intramolecular cyclization to form the benzo[d]imidazo[2,1-b]thiazole intermediate 4 (Scheme 6). The acid moiety of the left-hand side (LHS) of intermediate 4 (Scheme 6) was elaborated to the amides, by HATU mediated coupling with a variety of amines affording the amide intermediates 5 (Scheme 6). The final step of the synthetic sequence involves palladium catalyzed cross-coupling to introduce an aryl/heteroaryl component at the bromo substituent of the heterocyclic intermediate 5 (Scheme 6). Cross-coupling partners to introduce R² include various boronic acid/esters (Suzuki-Miyaura coupling) or various organostannane reagents (Stille coupling) to furnish the final compounds with various right-hand sides (RHS), Structure I (Scheme 6).

### Synthesis of benzo[d]imidazo[2,1-b]thiazole Compounds, Structure II

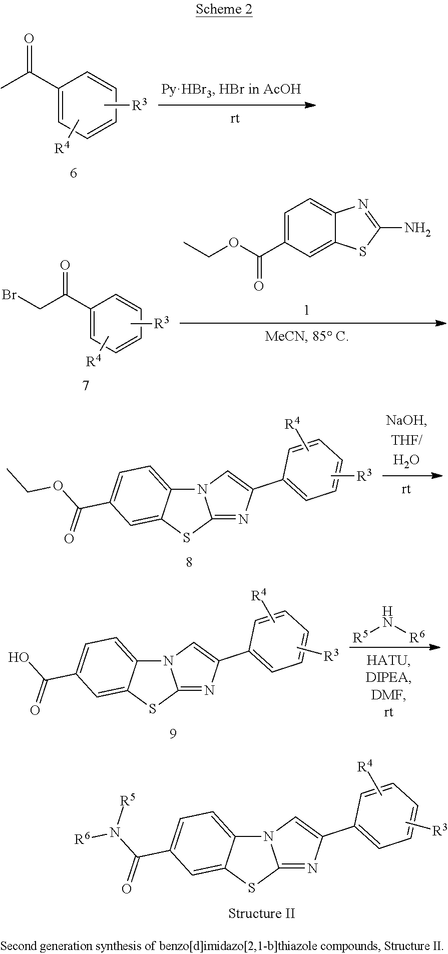

The first step of the synthesis involves bromination of the a-carbonyl position of various substituted aryl methyl ketones 6 (Scheme 7), using pyridinium tribromide in the presence of HBr in acetic acid affording substituted phenacyl bromide intermediates 7 (Scheme 7). These intermediates 7 (Scheme 7) facilitate ready diversification of the right-hand side (RHS) of the final compounds, Structure II. Intermediate 7 (Scheme 7) undergoes a alkylation reaction followed by intramolecular cyclization with ethyl 2-aminobenzothiazole-6-carboxylate 1 (Scheme 7) at elevated temperature to from ester benzo[d]imidazo[2,1-b]thiazole intermediate 8 (Scheme 7). Hydrolysis of ester intermediate 8 (Scheme 7) with sodium hydroxide in water/THF mixture affords acid intermediate 9 (Scheme 7). The final step involves an amide coupling of various primary\secondary amines with acid intermediate 9 (Scheme 7), using HATU as a coupling reagent delivering the final compounds with various left-hand side (LHS) amides, Structure II (Scheme 7).

### Alternative Synthesis of benzo[d]imidazo[2,1-b]thiazole Compounds, Structure II

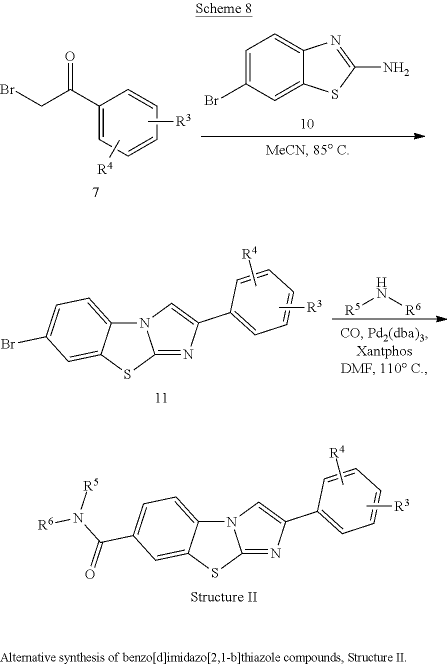

The first step involves a “one-pot” alkylation and intramolecular cyclization reaction between substituted phenacyl bromide intermediates 7 (as in Scheme 7) and 2-amino-6-bromobenzothiazole 10 (Scheme 8) at elevated temperature affording 7-bromo-2-aryl-lbenzo[d]imidazo[2,1-b]thiazole intermediates 11 (Scheme 8). The bromo heterocyclic intermediate 11 (Scheme 8) is employed as the key starting material for the final palladium-catalyzed aminocarbonylation reaction at elevated temperature. Various primary\secondary amines are used in this final palladium-catalyzed aminocarbonylation reaction to provide a variety of left-hand side (LHS) amides, Structure II (Scheme 8).

### Synthesis of Reverse Amide benzo[d]imidazo[2,1-b]thiazole Compounds, Structure III

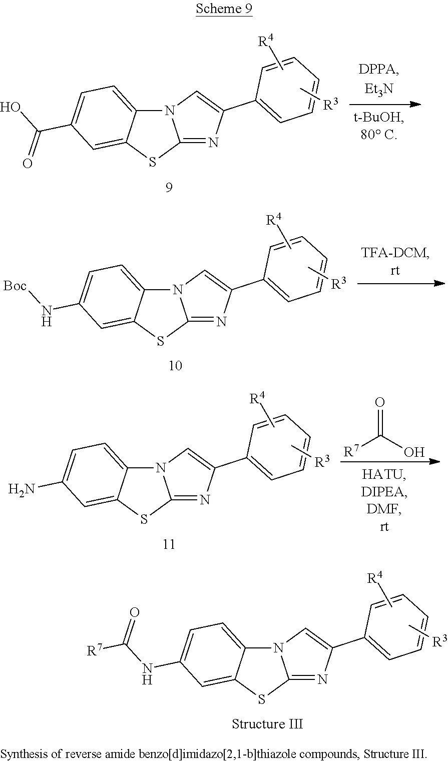

The first step of the synthesis proceeds via a Curtius Rearrangement, using diphenyl phosphoryl azide (DPPA) and tert-butanol in the presence of triethylamine at elevated temperature affording N-Boc amine intermediate 10 (Scheme 9). N-Boc deprotection of intermediate 10 (Scheme 9) using a mixture of TFA in DCM enabled ready access to the 7-amino-2-aryl-lbenzo[d]imidazo[2,1-b]thiazole intermediate 11 (Scheme 9). The final step involves amide coupling of the amine intermediate 11 (Scheme 9) with a variety of carboxylic acids, using HATU as a coupling reagent to furnish the desired left-hand side (LHS) reverse amides, Structure III (Scheme 9).

### Example 2

### General Synthetic Details for Additional Compounds of the Invention (Schemes 10-56)

### General Synthesis of Compounds of the Invention

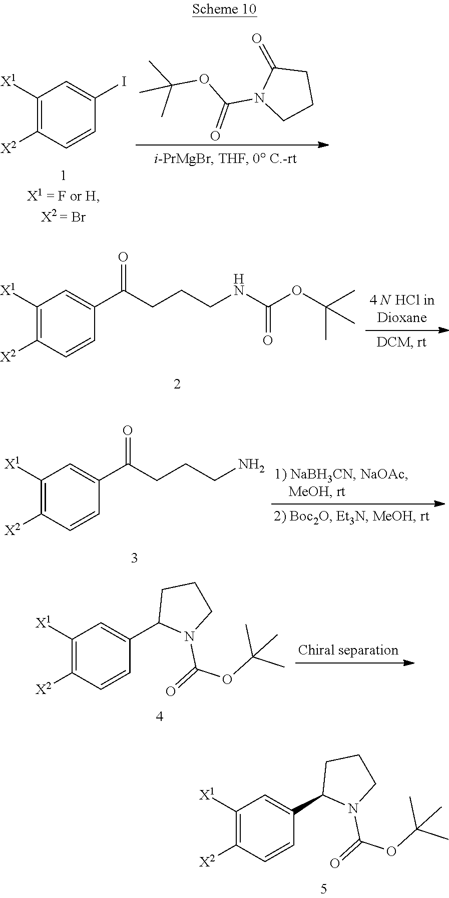

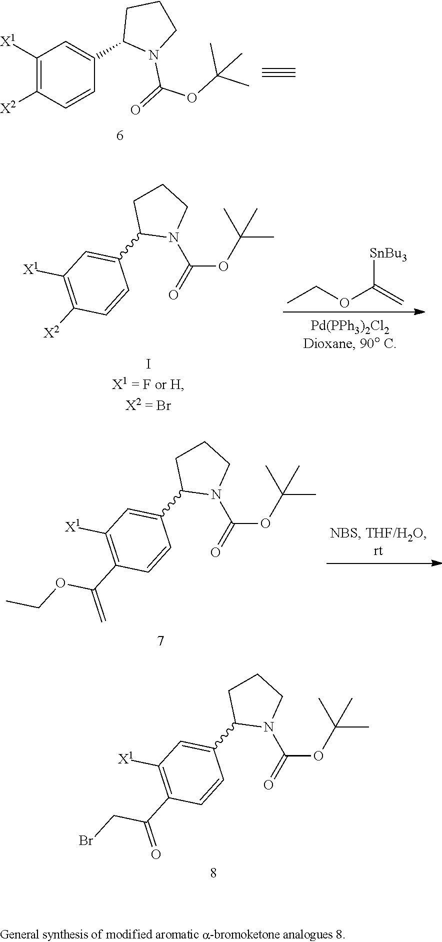

Compounds 1 were reacted with isopropylmagnesium bromide at low temperatures to generate the corresponding phenylmagnesium bromide, which were then treated with tert-butyl 2-oxopyrrolidine-1-carboxylate to generate ketones 2. The N-Boc protecting group of 2 was removed under acidic conditions to generate terminal amines 3 as the hydrochloride salts. Then an intramolecular reductive amination reaction was carried out and the resulting endo-amines were protected as the Boc carbamates in-situ to generate intermediates 4. Separation by chiral resolution gave two enantiomers 5 and 6. Intermediates 4, 5 and 6 could be expressed as common compounds I. Compounds I were reacted with tributyl(1-ethoxyvinyl)stannane in the presence of catalytic palladium at elevated temperature to generate vinyl ethyl ethers 7. These were treated with N-bromosuccinimide in aqueous conditions to generate aromatic a-bromoketone analogues 8.

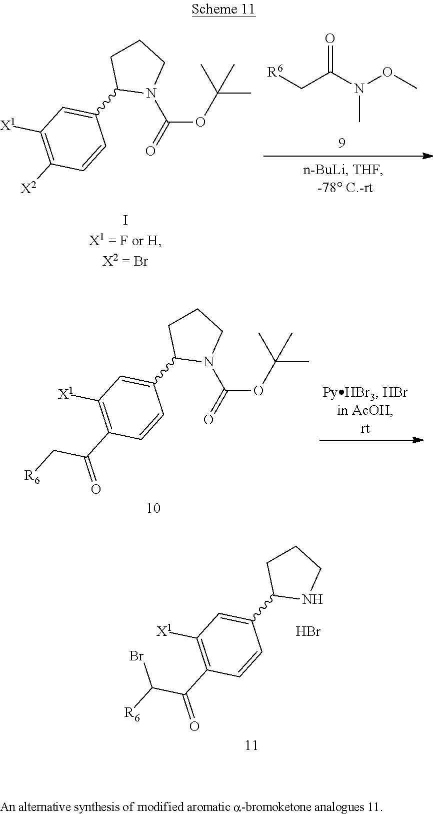

Lithium-halogen exchange was carried out on Compounds I using n-butyl lithium at low temperature. Subsequent trapping with various Weinreb amides 9 generated ketones 10. These were brominated by pyridinium tribromide to generate additional aromatic a-bromoketone analogues 11.

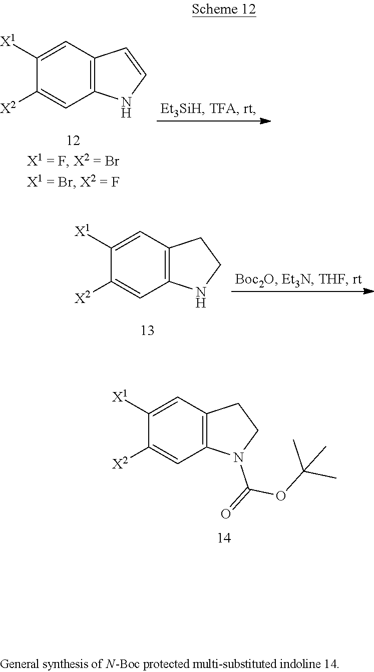

Substituted indoles 12 were reduced using triethylsilane under acidic conditions to afford indolines 13. The resulting anilines were protected by Boc group to generate intermediates 14.

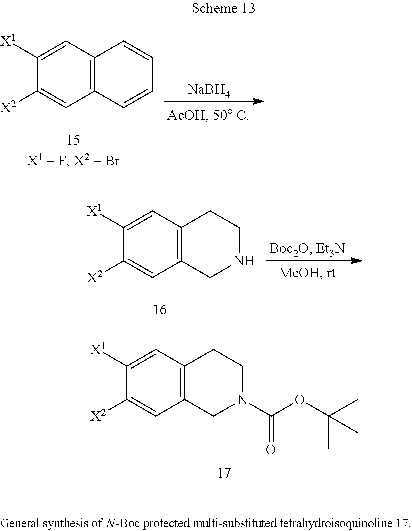

Substituted isoquinoline 15 was reduced to 1,2,3,4-tetrahydroisoquinoline 16. The resulting amine 16 was protected as the Boc carbamate to generate intermediate 17.

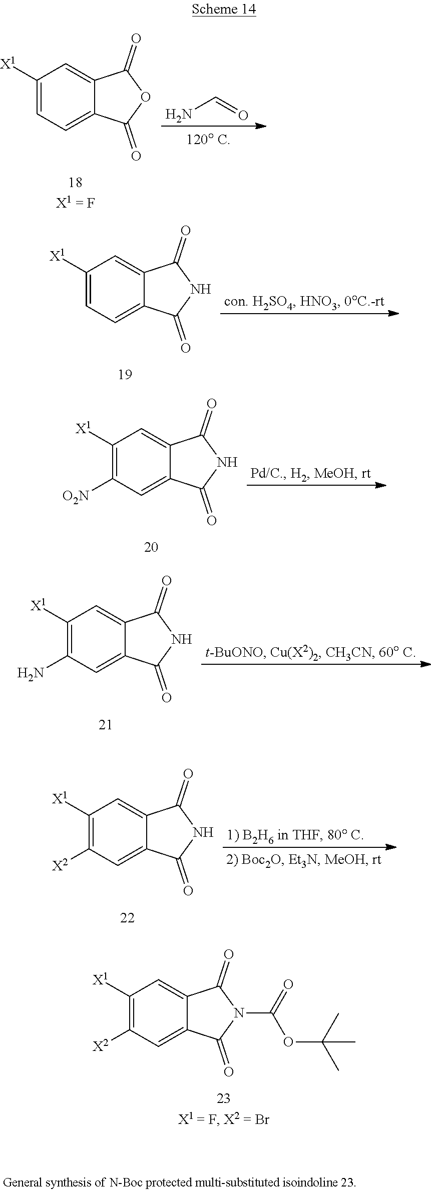

A reaction of 5-halidoisobenzofuran-1,3-dione 18 with formamide at elevated temperature afforded 5-halidoisoindoline-1,3-dione intermediate 19. Then a nitration reaction was carried out to generate intermediate 20. The nitro group of 20 was reduced by hydrogenation generating intermediate 21. The aniline was converted to halide with t-butyl nitrite and copper(II) halide to provide intermediate 22. Subsequent reduction reaction using diborane in tetrahydrofuran at elevated temperature followed by protection of the resulting endocyclic amine with a Boc group in-situ afforded intermediate 23 to complete the synthesis.

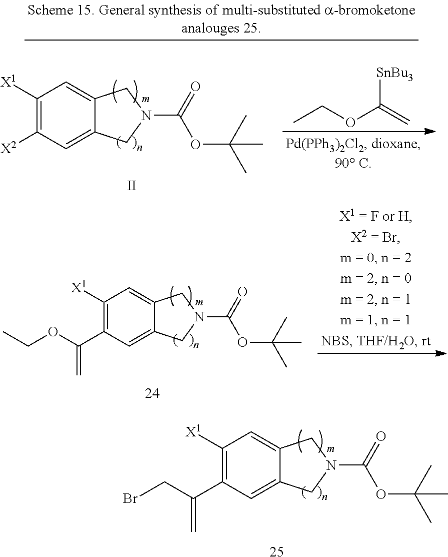

Intermediates 14, 17 and 23 could be expressed as common compounds II, which were used as starting materials following the same synthetic sequence as in Scheme 10, to generate the third a-bromoketone analogues 25.

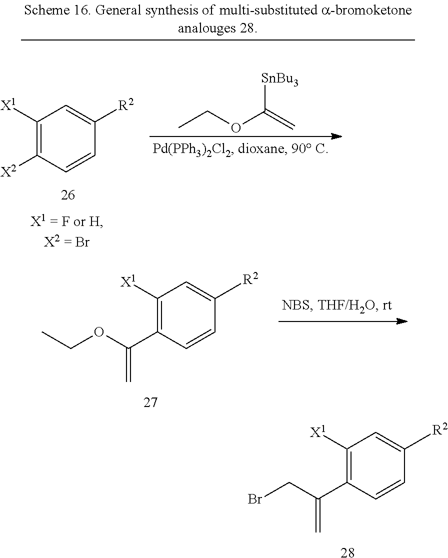

The fourth a-bromoketone analogues 28 were synthesized following the same synthetic sequence as in Scheme 10 using starting materials 26.

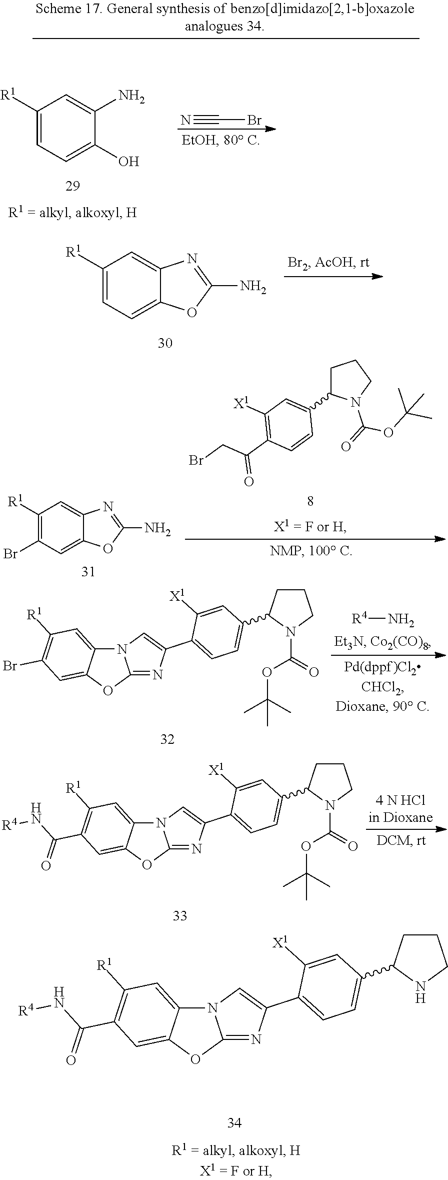

2-aminophenol compounds 29, were cyclized by reaction with cyanic bromide. A subsequent bromination reaction was performed to generate intermediates 31. This was followed by another cyclization with a-bromoketone analogues 8 at elevated temperature to generate intermediates 32. The bromide of intermediates 32 was converted to various amides via aminocarbonylation reaction to generate intermediates 33. The N-Boc protecting group was removed using 4 N hydrochloride in 1,4-dioxane to produce the final benzo[d]imidazo[2,1-b]oxazole analogues 34 as a free base or a hydrochloride salt.

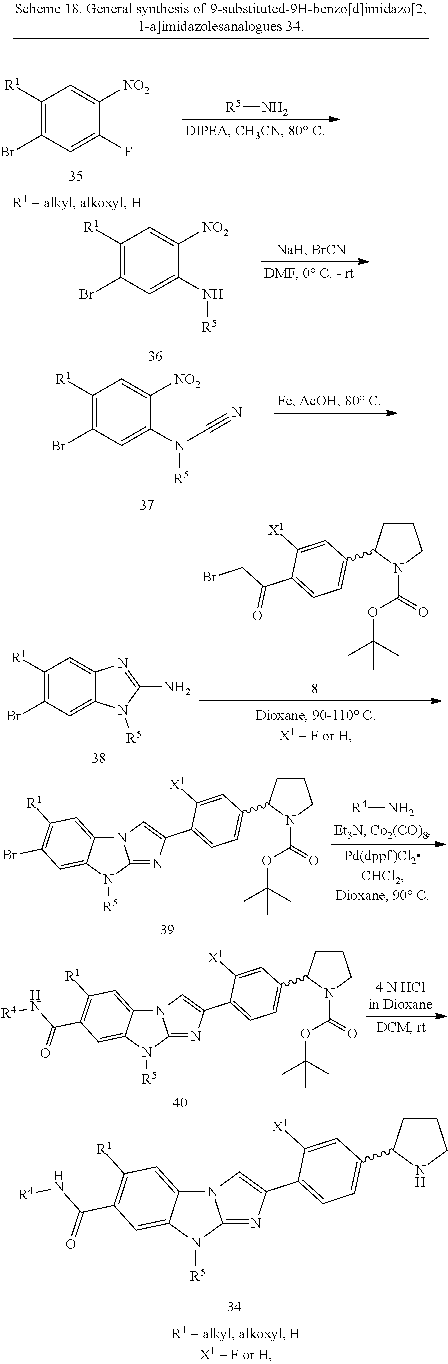

The fluoride of 2-fluoronitrobenzene compounds 35 was substituted by various amines to generate secondary anilines 36. These were reacted with cyanic bromide using strong base to generate phenylcyanamides 37. Intermediates 37 go through a reductive cyclization reaction to generate 2-aminobenzo[d]imidazoles 38. Final compounds 41 were synthesized as the free base or as the hydrochloride salt from intermediates 38 following the same synthetic sequence as in Scheme 17

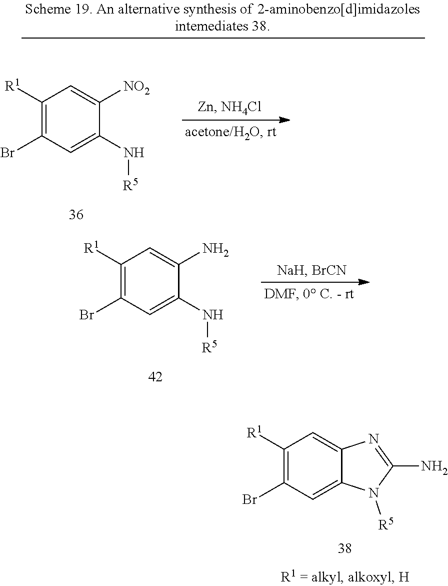

The nitro group of intermediates 36 were reduced by zinc powder to generate benzene-1,2-diamine intermediates 42. These were reacted with cyanic bromide under strong base to generate 2-aminobenzo[d]imidazoles intermediates 38.

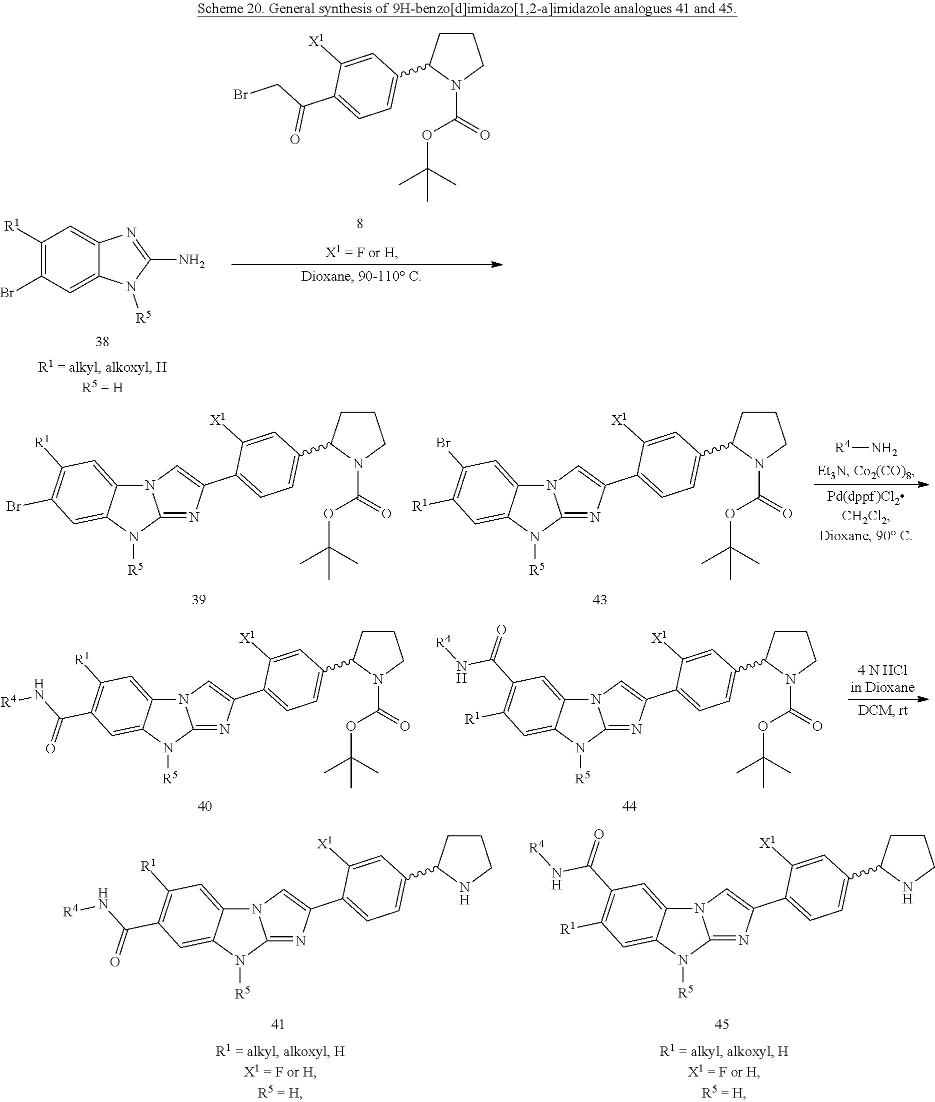

Regioisomers 39 and 43 were formed when intermediates 38 R⁵═H. The mixture of bromide intermediates 39 and 43 were converted to the corresponding amides 40 and 44 following the description in Scheme 18 step 5. The amide diastereomers 40 and 44 were separated by Chiral resolution. The N-Boc group of each isomer was removed under acidic conditions to generate final 9H-benzo[d]imidazo[1,2-a]imidazole analogues 41 and 45 respectively as the free base or salt.

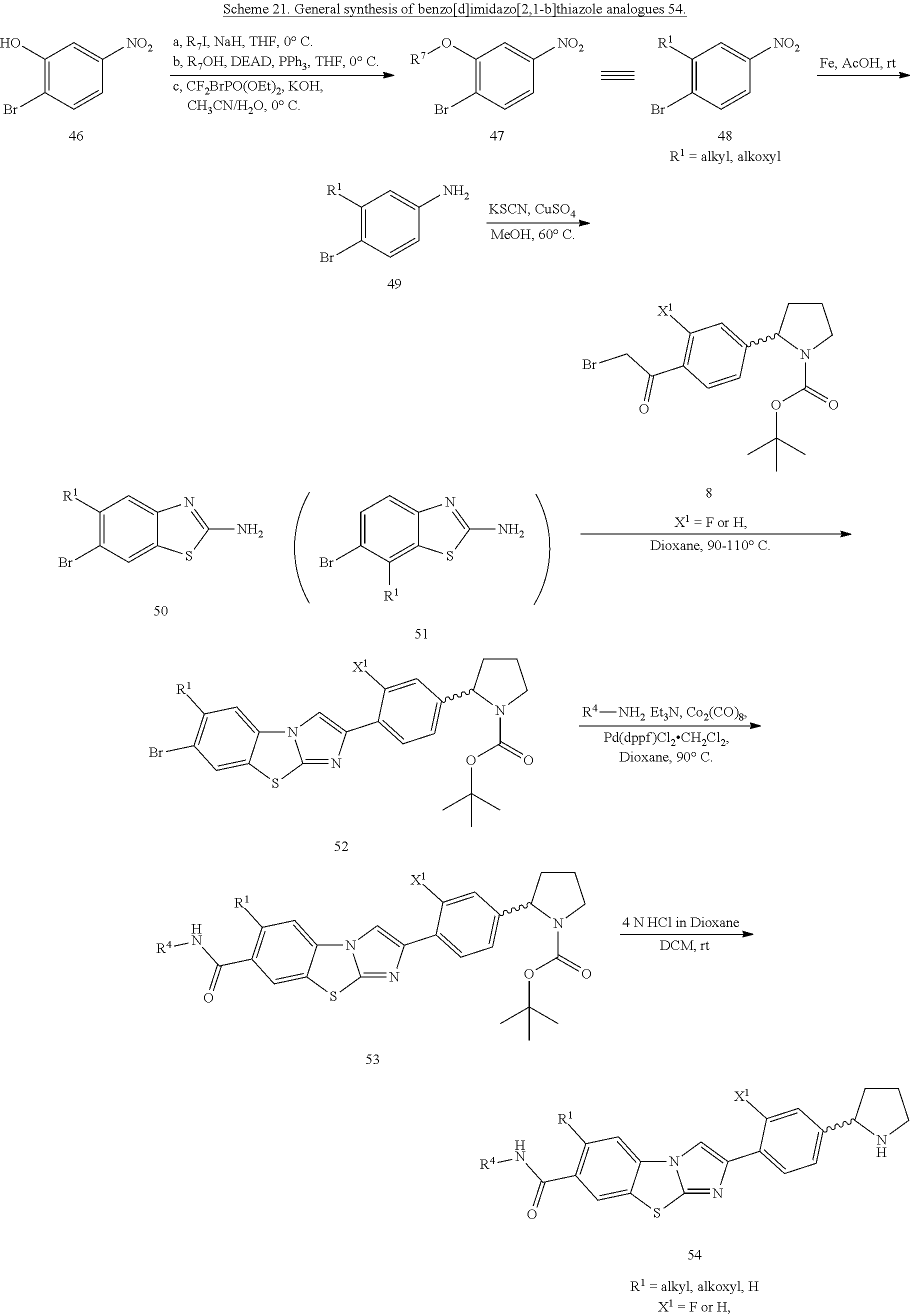

An etherification of phenol compounds 46 through substitution or Mitsunobu reaction gave phenyl alkyl ether intermediates 47, which could be expressed as compounds 48. The nitro group of 48 was reduced by iron powder to generate aniline intermediates 49. Then a cyclization reaction was carried out on 49 with potassium thiocyanate catalyzed by copper sulfate to generate 2-amine benzothiazole regioisomers 50 and 51 with various ratios according to the steric hindrance of R¹ group. Compounds 50 were converted to the final benzo[d]imidazo[2,1-b]thiazole analogues 54 as free base or salt in the same manner as in Scheme 17. Chiral resolution was performed on amides 53 to generate final enantiomers if required.

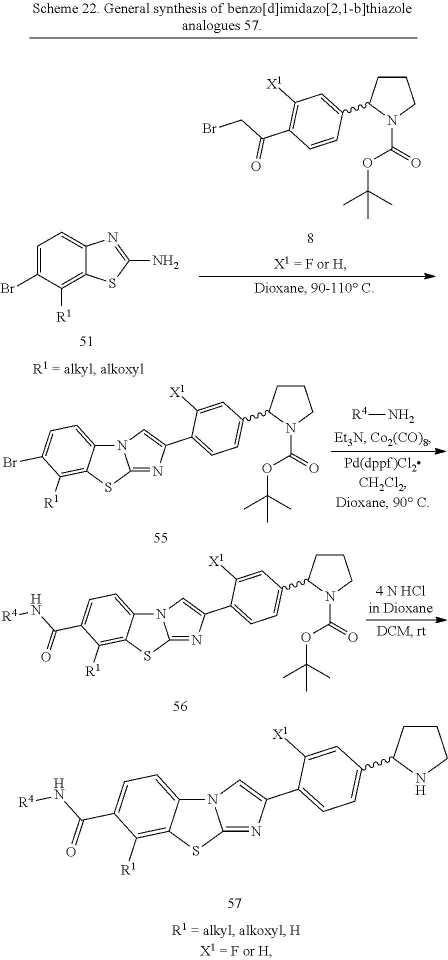

Final compounds 57 were synthesized from regioisomers 51 following the same description as in Scheme 21.

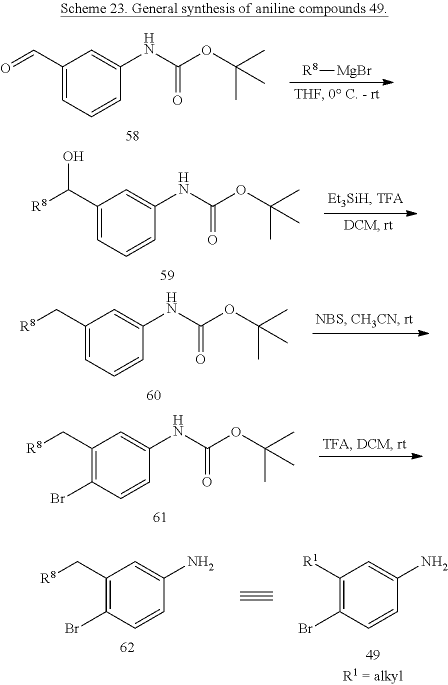

Addition of Grignard reagents to benzaldehyde 58 gave benzyl alcohol compounds 59. Reduction using triethylsilane/trifluoroacetic acid resulted intermediates 60. These were brominated with N-bromosuccinimide to generate intermediates 61. The N-Boc protecting group was removed under acidic conditions to generate common compounds 49.

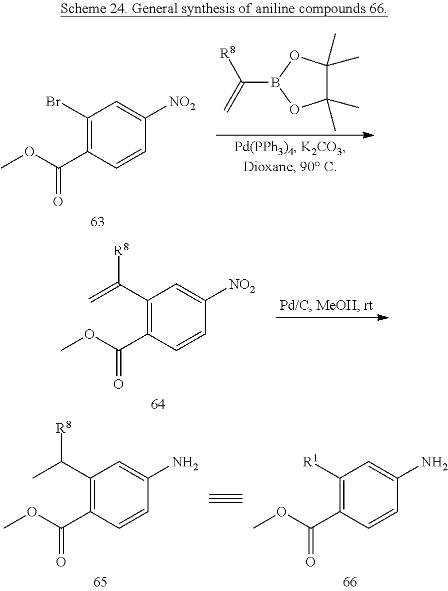

Substituted olefins were introduced to compound 63 via Suzuki cross-coupling reaction to generate intermediates 64. Reduction of both the olefin and nitro group by hydrogenation gave the common aniline compounds 66.

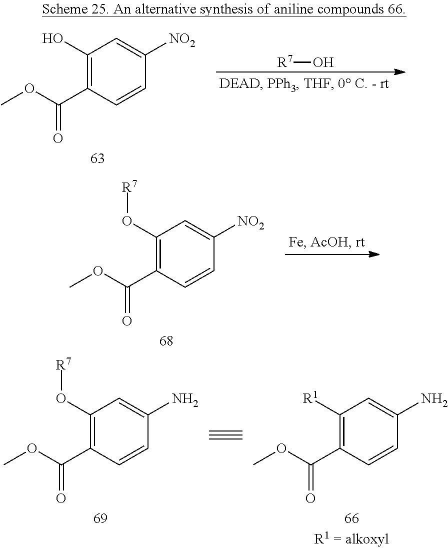

Etherification of compound 67 via Mitsunobu reaction generated ether compounds 68. Nitro group was reduced by iron powder to generate common aniline compounds 66.

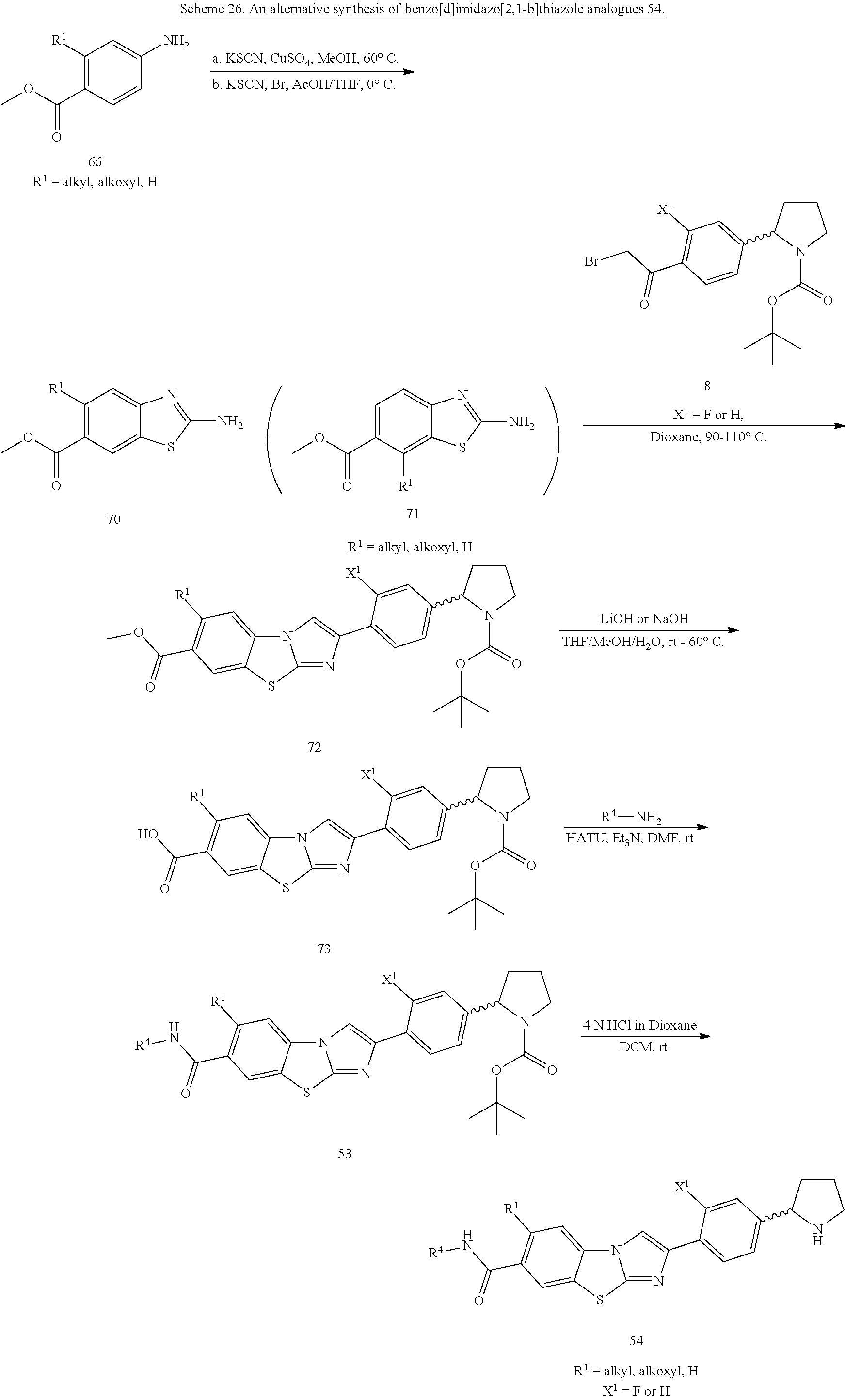

Ester compounds 72 were synthesized from aniline compounds 66 following the descriptions in Scheme 21. The ester was hydrolyzed to form carboxylic acids 73. Amide coupling reaction on 73 with various amines gave amides 53. The N-Boc protecting group was removed by 4 N hydrochloride in 1,4-dioxane to generate benzo[d]imidazo[2,1-b]thiazole analogues 54 as a free base or as the hydrochloride salt.

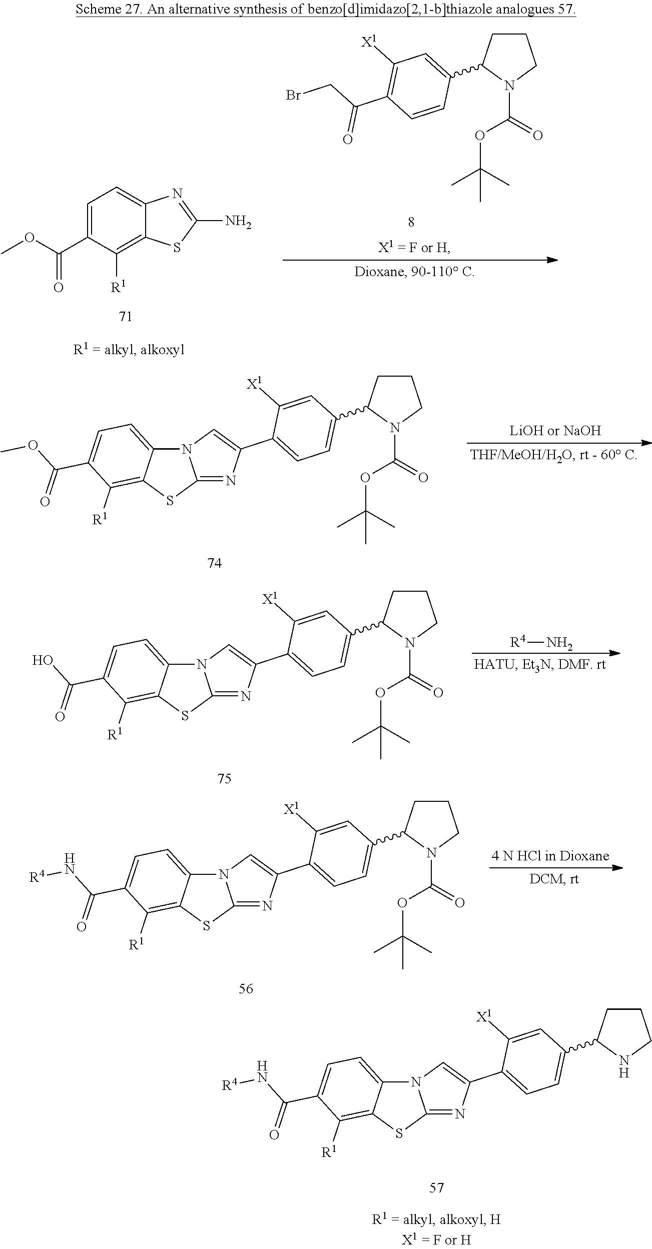

Final compounds 57 as free base or hydrochloride salt, were synthesized from regioisomers 71 following the same synthetic sequence as in Scheme 26.

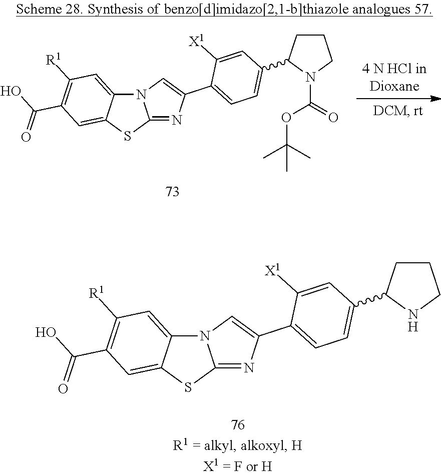

The N-Boc protecting group of 73 was directly removed to generate compounds 76 as a free base or salt.

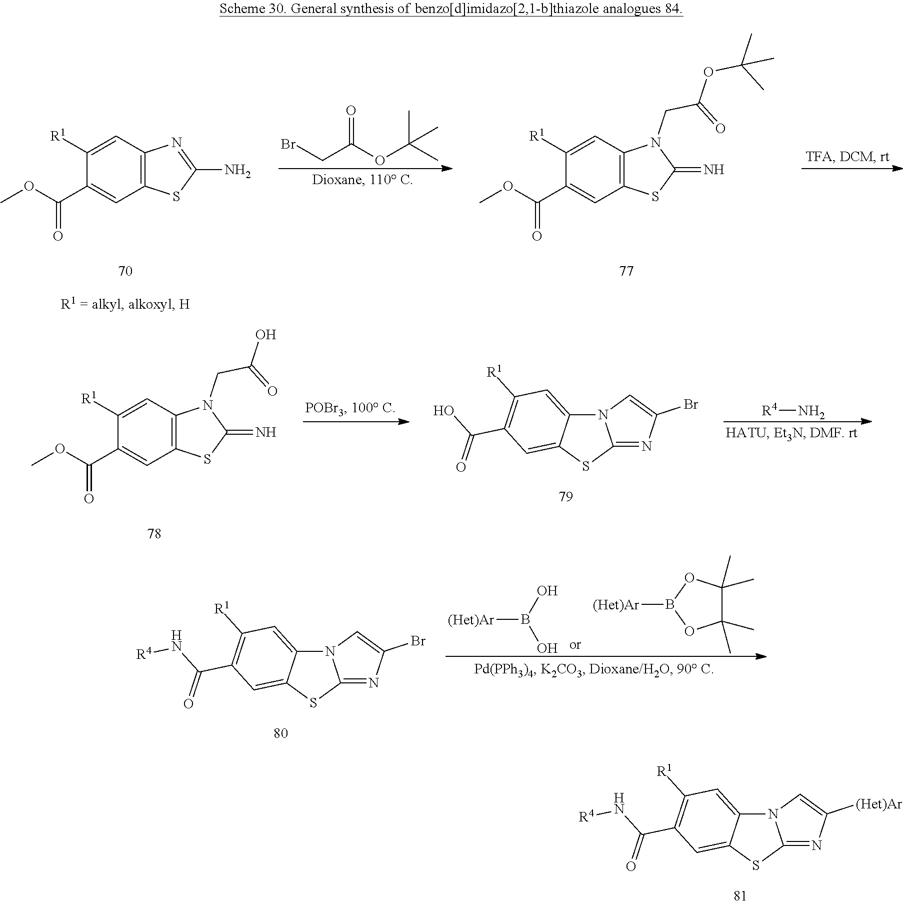

Compounds 70 were reacted with tert-butyl 2 bromoacetate at elevated temperature to generate intermediates 77. The tert-butyl ester of 77 was hydrolyzed by trifluoroacetic acid to generate carboxylic acid compounds 78, which were treated with phosphoryl tribromide to generate bromo acid compounds 79. Amide coupling reaction of 79 with various amines generated bromo amide common intermediates 80. A Suzuki cross-coupling reaction of 80 with aromatic boronic acids or borates produced the final benzo[d]imidazo[2,1-b]thiazole analogues 81 as a free base or as the hydrochloride salt.

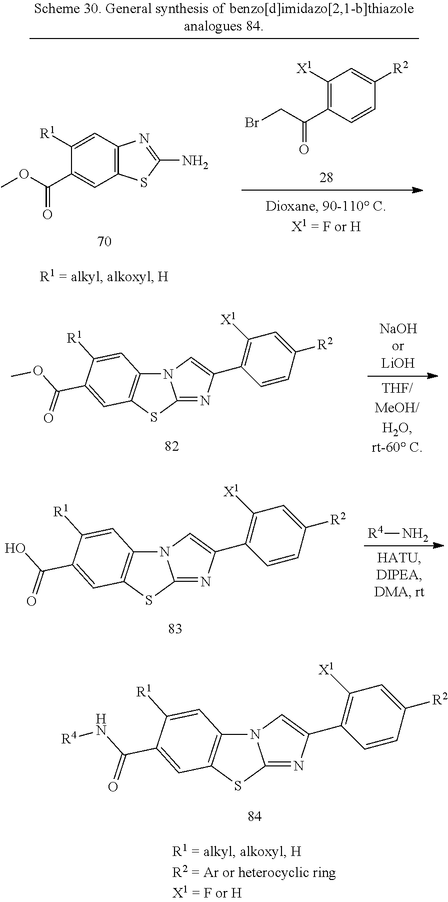

Final compounds 84 were synthesized from compounds 70 following the same synthetic sequence as in Scheme 26.

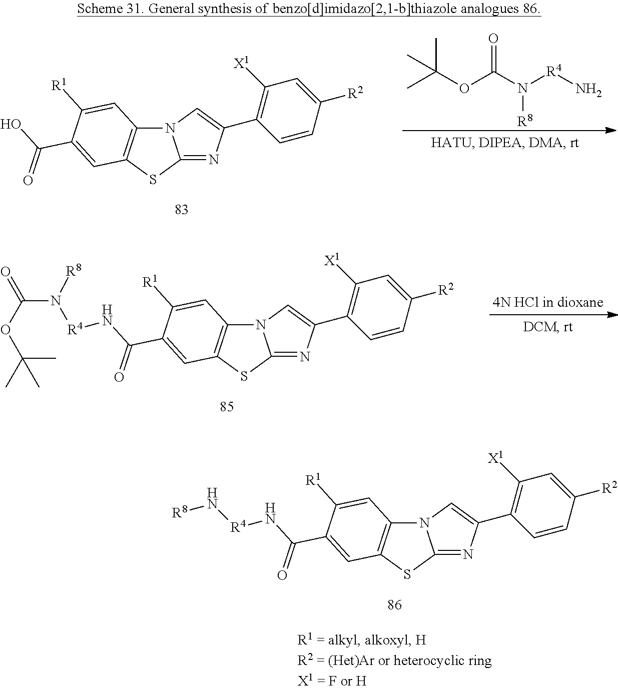

An amide coupling reaction was performed on acids 83 with various amines to generate amides 85. The N-Boc protecting group of 85 was removed to generate benzo[d]imidazo[2,1-b]thiazole analogues 86 as the free base or as a hydrochloride salt.

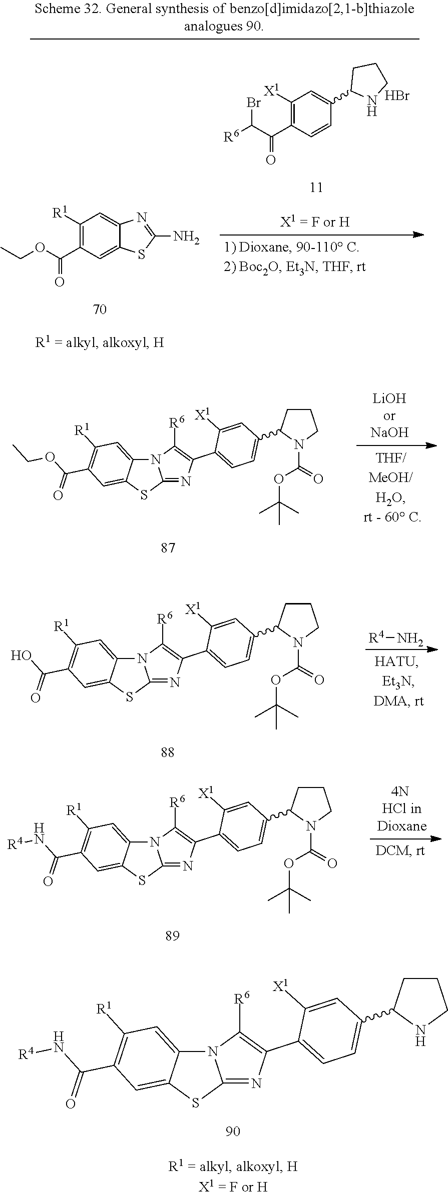

The first step of the synthesis involves a cyclization followed by protecting the endo-amine as the Boc carbamate in-situ to generate esters 87. The final compounds 90 were isolated as the free base or as the hydrochloride salt following the same synthetic sequence as in Scheme 26. Chiral resolution was performed on amide compounds 89 if required.

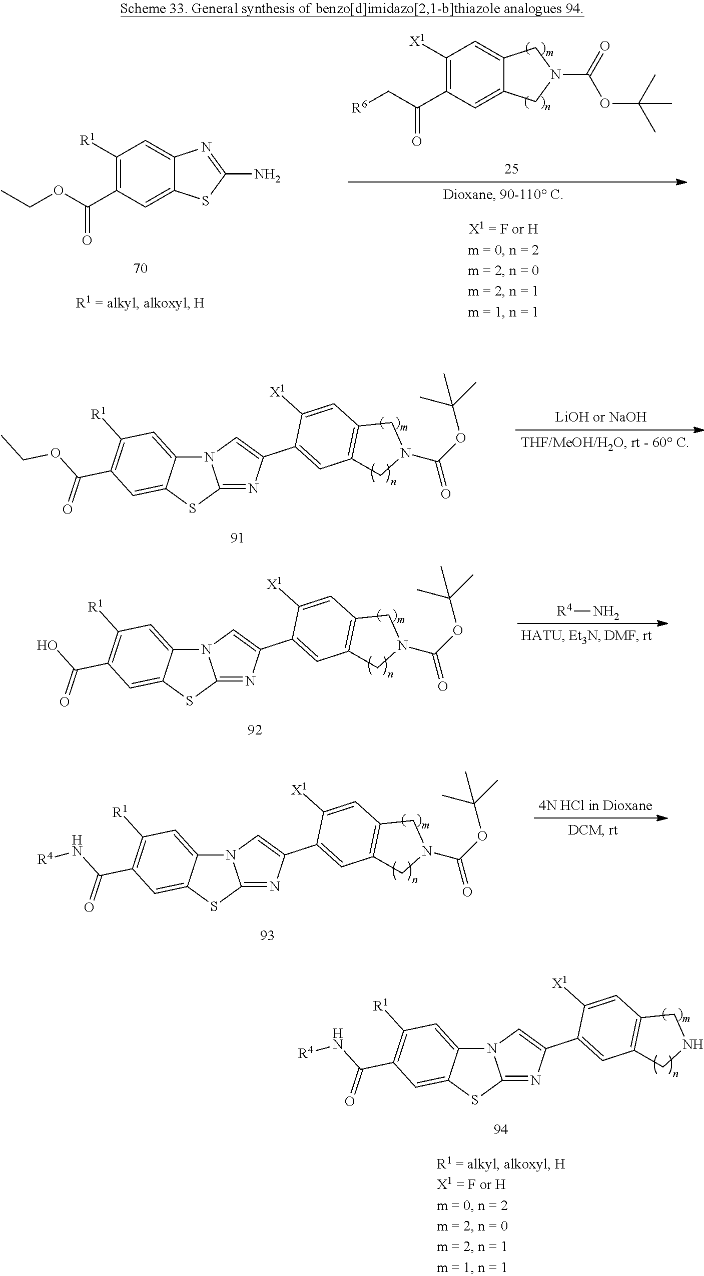

The final compounds 94 were synthesized from compounds 70 following the same synthetic sequence as in Scheme 26. The compounds were isolated as the free base or as the hydrochloride salt.

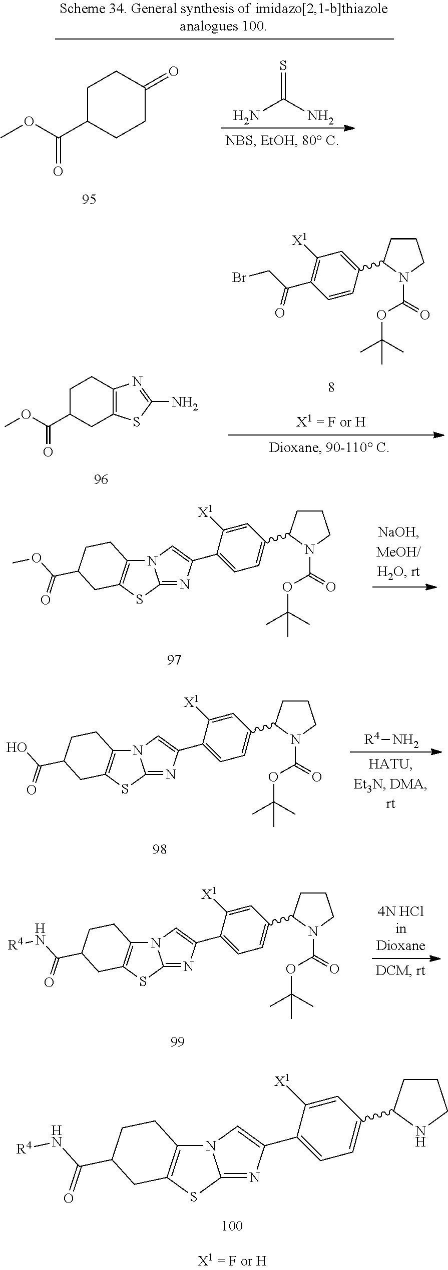

A cyclisation reaction with ketone 95 and thiourea gave 2-aminothiazole 96. The final compounds 100 were isolated as the free base or as the hydrochloride salt, following the same synthetic sequence applied in Scheme 26.

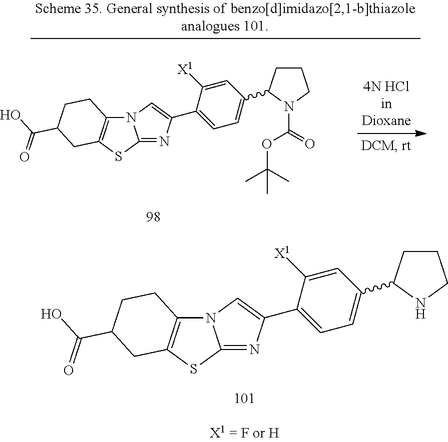

The N-Boc of compounds 98 was removed to generate compounds 101 as the free base or as a hydrochloride salt.

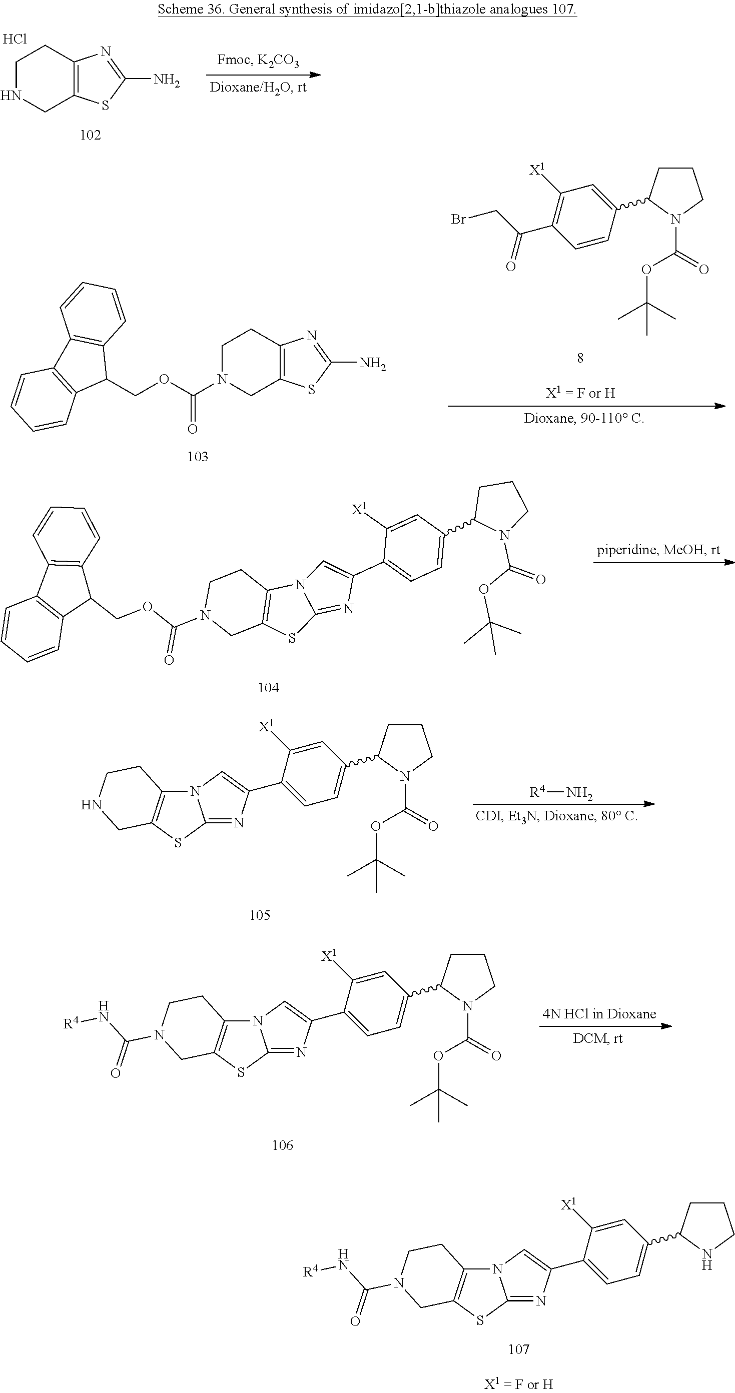

The cyclic amine of compound 102 was protected as the Fmoc carbamate to generate compound 103. It was reacted with intermediates 8 to generate intermediates 104. The Fmoc group was removed with piperidine to generate unprotected amine 105. Urea 106 was formed from amine 105 and various amines. The N-Boc protecting group was removed to generate final compounds 107 as a free base or as the hydrochloride salt.

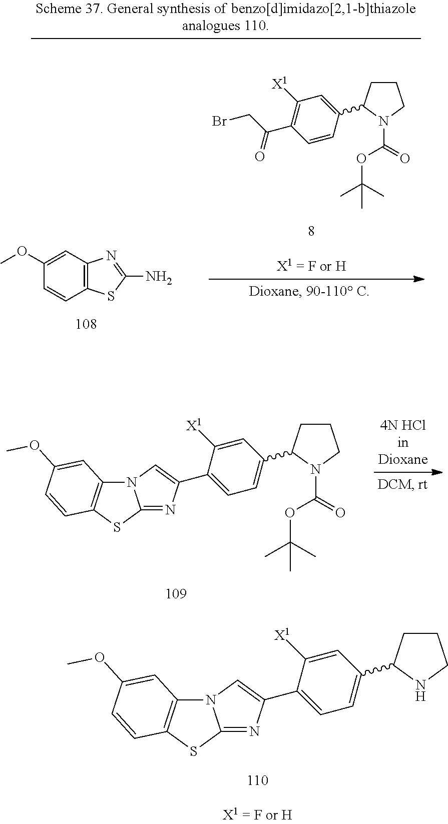

Compounds 110 were synthesized from commercial available 5-methoxybenzo[d]thiazol-2-amine, 108, following the same synthetic sequence as in Scheme 26. The compounds were isolated as the free base or as the hydrochloride salt.

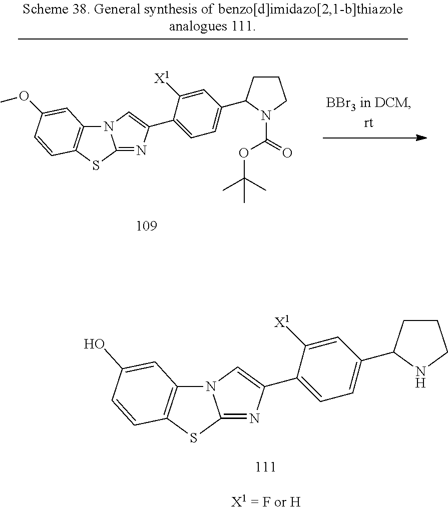

Methyl group of compounds 109 was removed by tribromoborane to generate compounds 111 as the free base or as the hydrochloride salt.

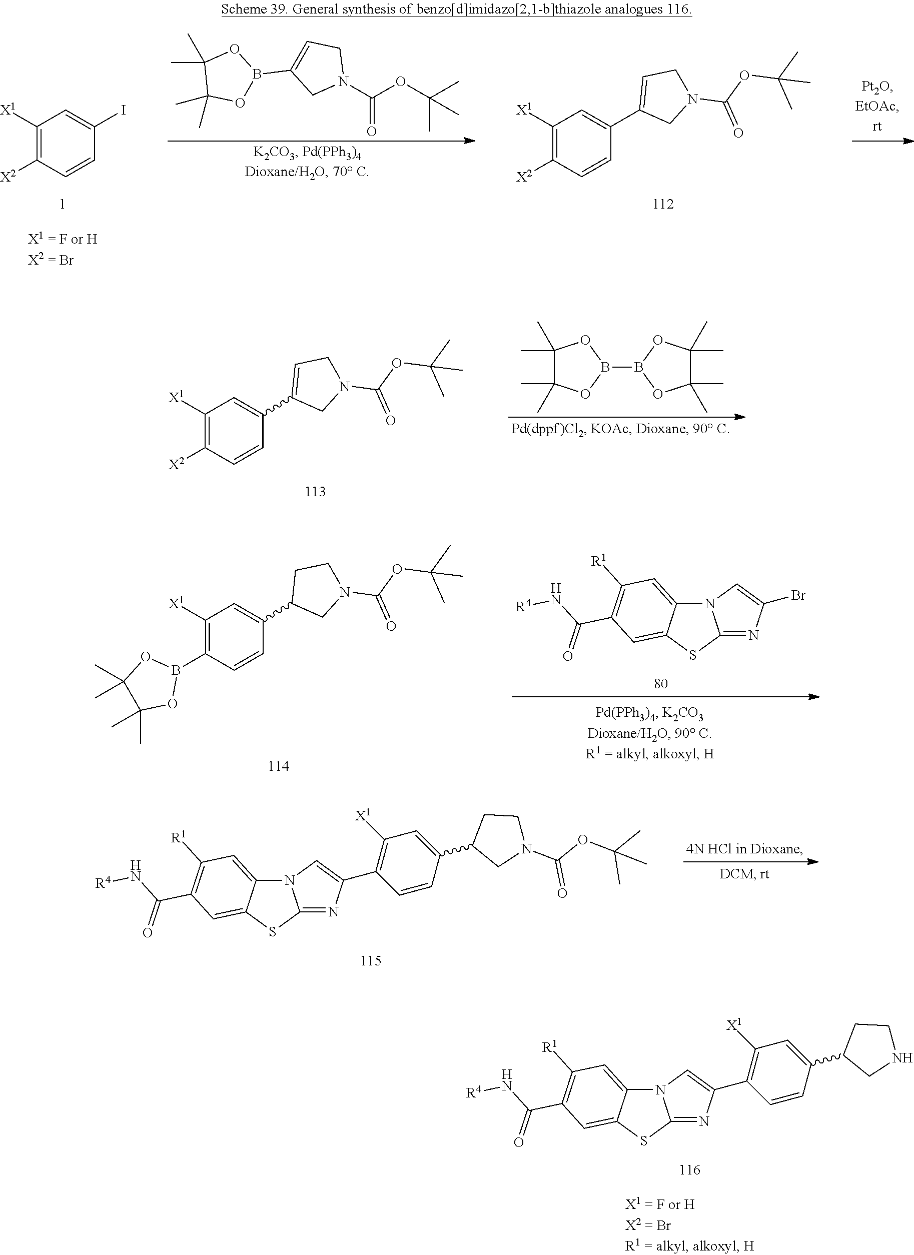

A Suzuki cross-coupling reaction of 1 with tert-butyl 3-(4,4,5,5-tetramethyl-1,3,2-dioxaborolan-2-yl)-2,5-dihydro-1H-pyrrole-1-carboxylate produced the olefin intermediates 112. Reduction of the olefin by hydrogenation gave intermediates 113. The Miyaura borylation reaction on intermediates 113 generated boronic ester intermediates 114, which were subject to a second Suzuki cross-coupling reaction with intermediates 80 to produce intermediates 115. The N-Boc protecting group was removed produced final compounds 116 as the free base or as the hydrochloride salt. Chiral resolution was performed on amides 115 to generate final enantiomers if required.

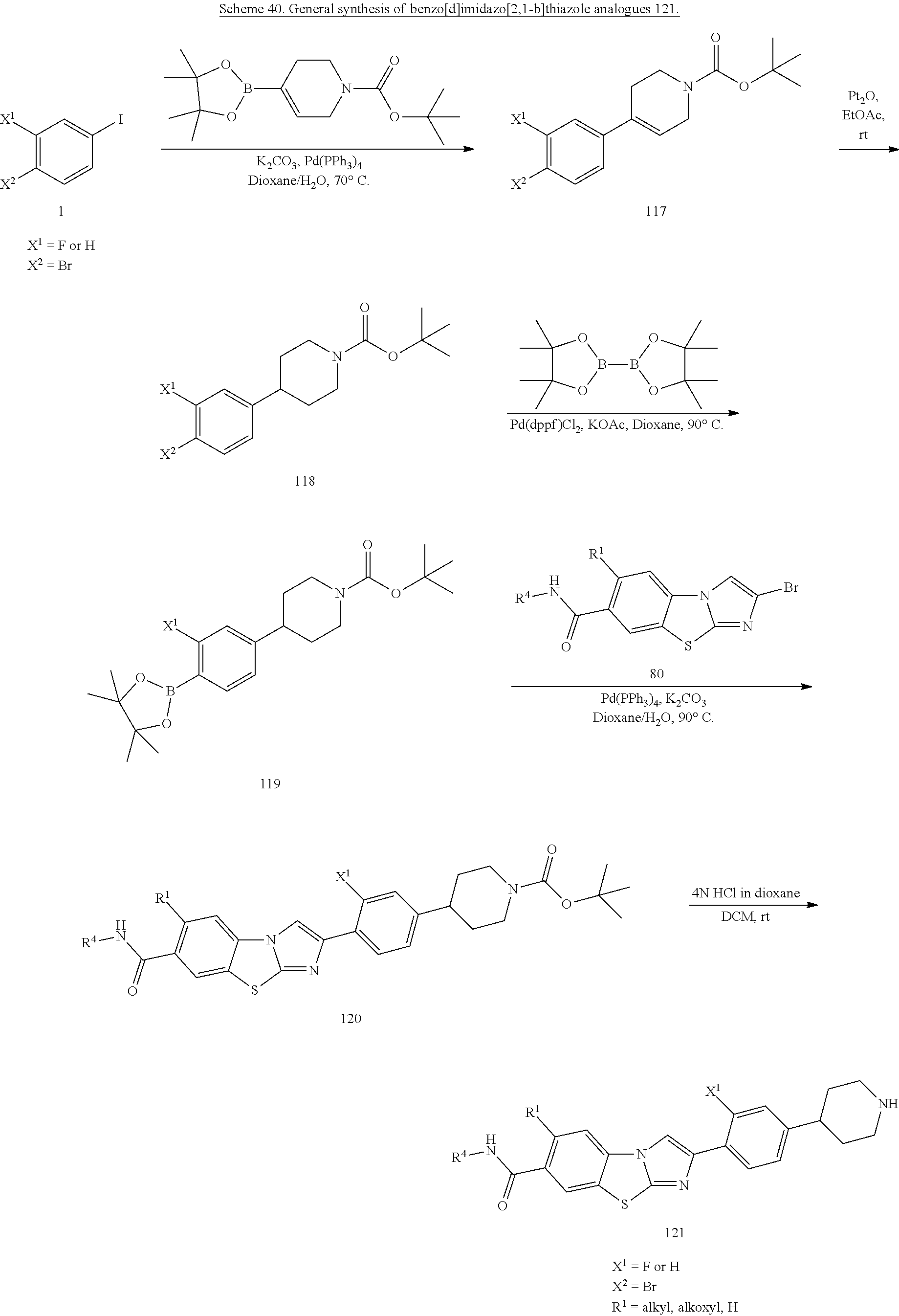

The final compounds 121 were synthesized from compounds 1 following the same synthetic sequence as in Scheme 39. The compounds were isolated as the free base or as the hydrochloride salt.

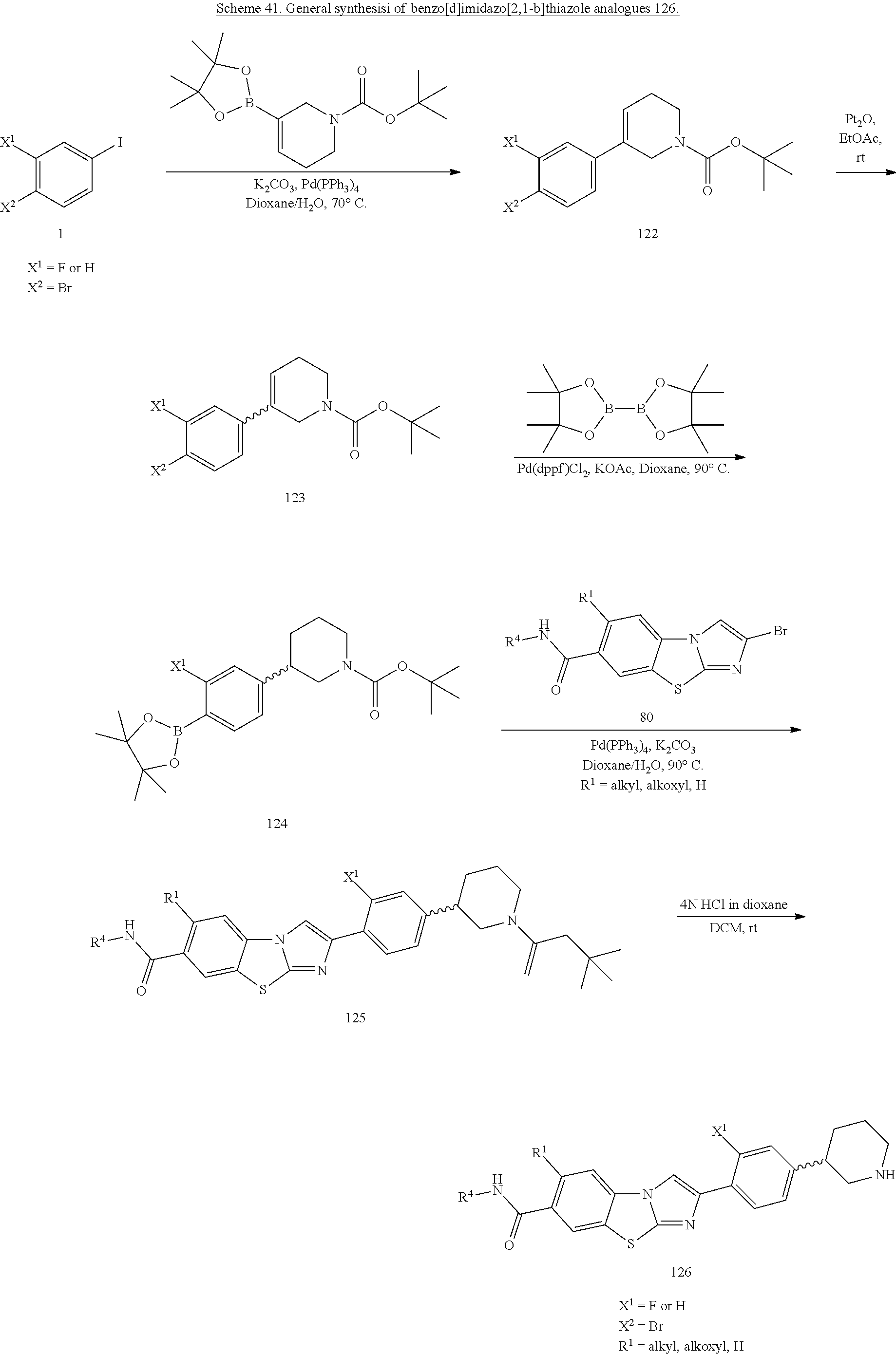

The final compounds 126 were synthesized from compounds 1 following the same synthetic sequence as in Scheme 39. The compounds were isolated as the free base or as the hydrochloride salt. Chiral resolution was performed on amides 125 to generate final enantiomers if required.

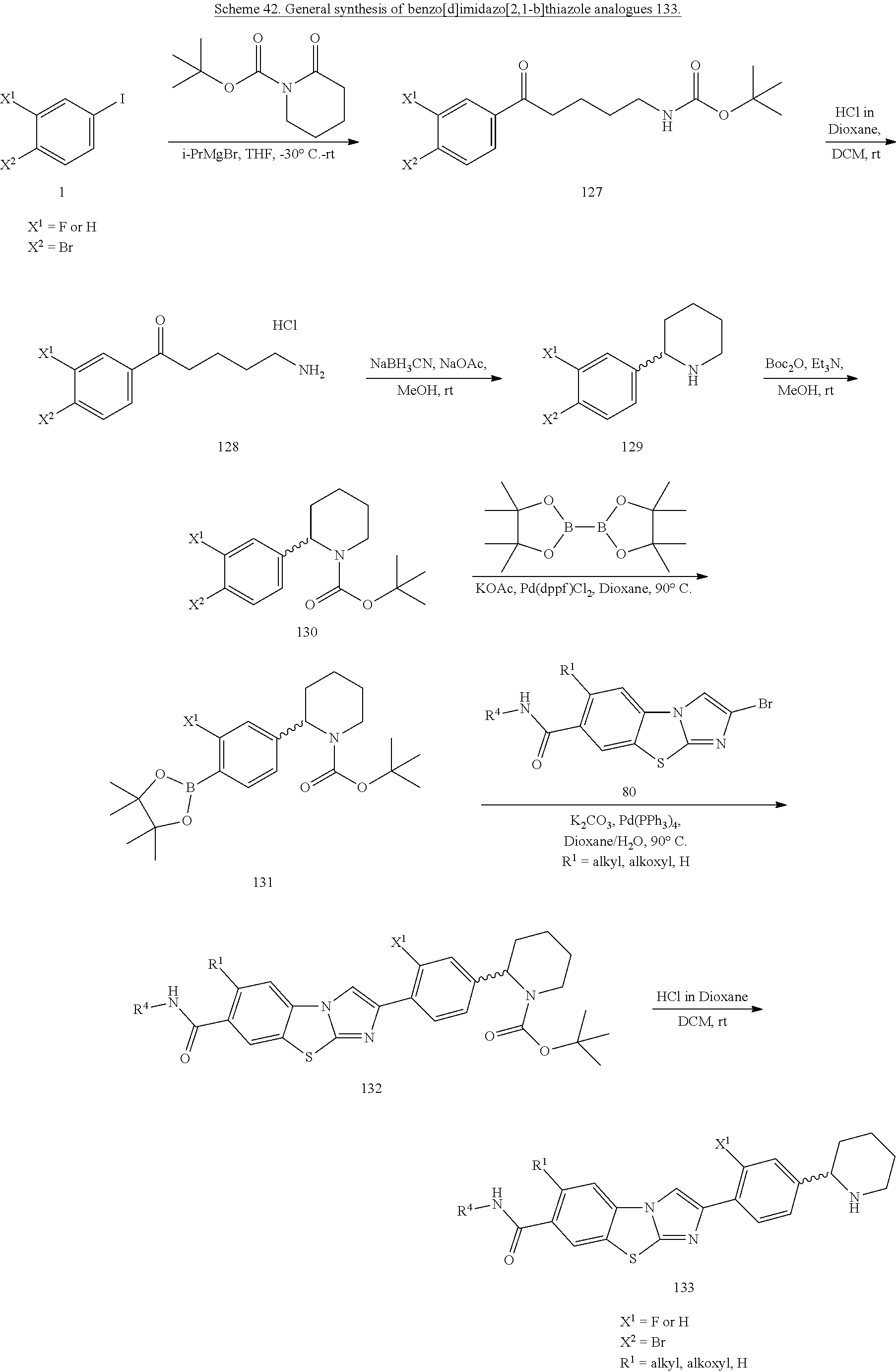

Intermediates 130 were synthesized from compounds 1 following the same synthetic sequence as in Scheme 10. The final compounds 133 were synthesized from compounds 130 following the same synthetic sequence as in Scheme 39. The compounds were isolated as the free base or as the hydrochloride salt. Chiral resolution was performed on amides 132 to generate final enantiomers if required.

### Synthetic Details for Compounds of the Invention

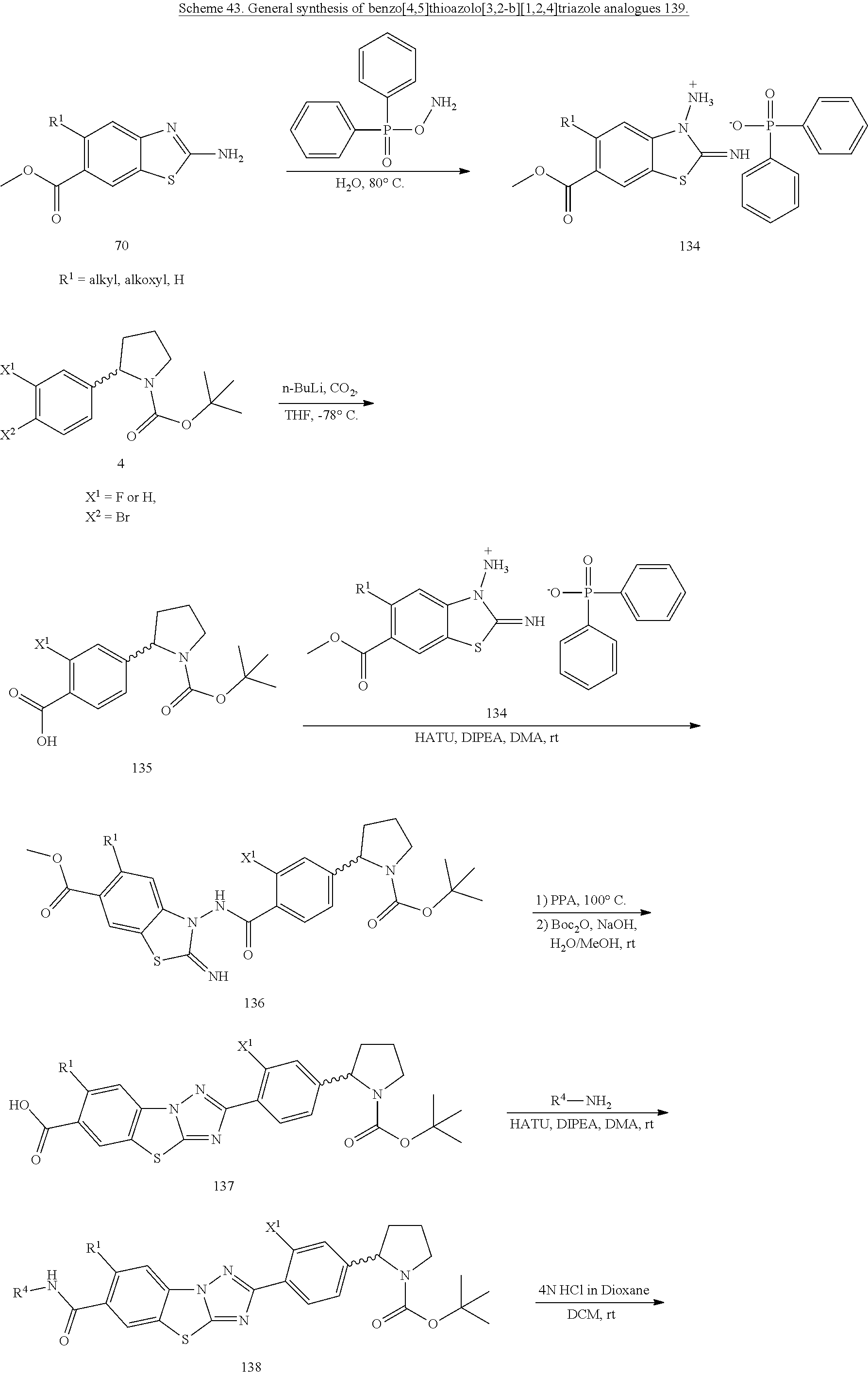

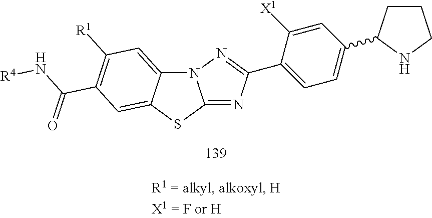

Common intermediates ammonium salts 134 were synthesized from 2-aminobenzothiazoles 70 and (aminooxy)diphenylphosphine oxide. Intermediates 4 were subjected to lithium-halogen exchange with n-butyl lithium at low temperature followed by treatment with dry ice to yield carboxylic acid intermediates 135. A condensation reaction between 135 and 134 generated amide intermediates 136. Intramolecular cyclization reaction was performed in polyphosphoric acid and then the N-Boc protecting group was reintroduced in-situ to generate carboxylic acids 137. The final compounds 139 were synthesized from intermediates 137 following the same synthetic sequence as in Scheme 26. The compounds were isolated as the free base or as the hydrochloride salt.

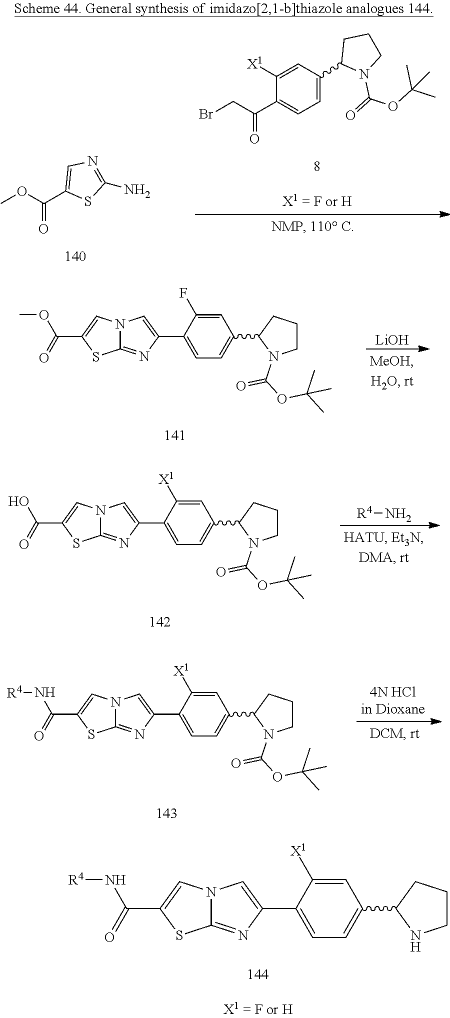

The final compounds 144 were synthesized from compounds 140 following the same synthetic sequence as in Scheme 26. The compounds were isolated as the free base or as the hydrochloride salt.

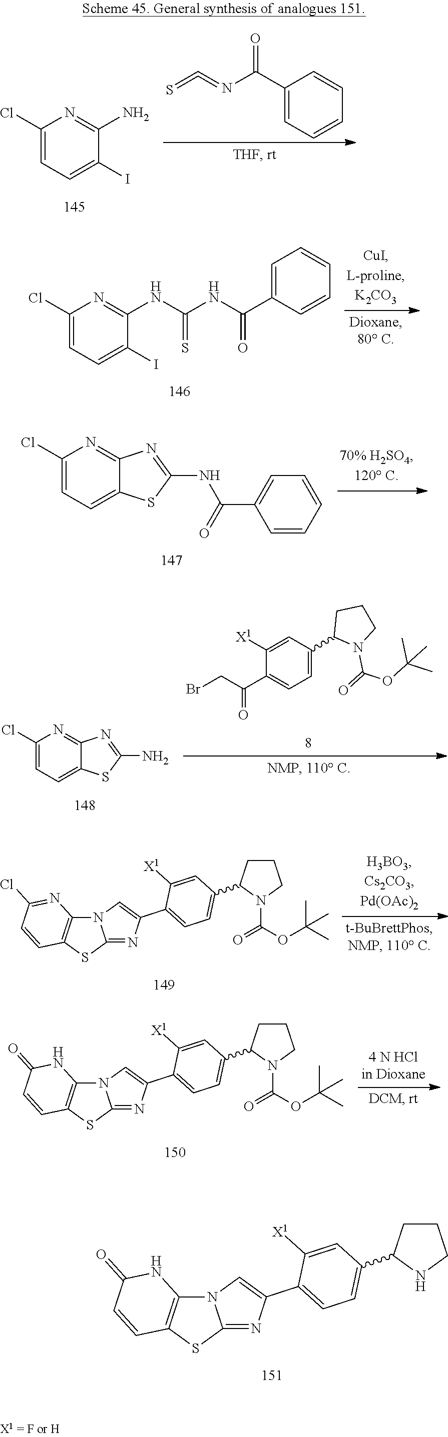

Compound 145 was reacted with benzoyl isothiocyanate to generate thiourea intermediates 146. An intramolecular cyclization reaction catalyzed by copper iodide produced intermediates 147. Hydrolysis under acidic conditions generated thiazole intermediates 148. Cyclization with compound 8 at elevated temperature generated intermediates 149. The o-chloride of the pyridine ring was hydrolyzed by boric acid/palladium catalyst system to generate pyridone compounds 150. The N-Boc protecting group was removed to produce final compounds 151 as the free base or as the hydrochloride salt. Chiral resolution was performed on N-Boc protected compounds 150 to generate final enantiomers if required.

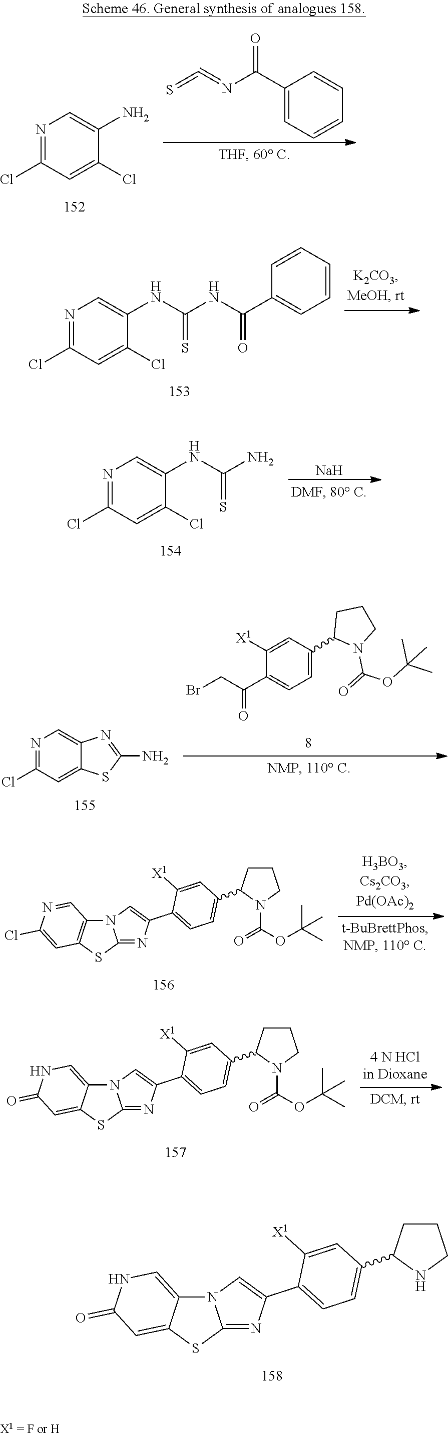

Intermediate 153 was synthesized from compound 152 and benzoyl isothiocyanate. The benzoyl group was removed by potassium carbonate/methanol treatment to generate thiourea 154. Treatment with strong base at elevated temperature produced cyclized intermediate 155. The final compounds 158 were synthesized from compound 155 following the same synthetic sequence as in Scheme 45. The compounds were isolated as the free base or as the hydrochloride salt. Chiral resolution was performed on N-Boc protected compounds 157 to generate final enantiomers if required.

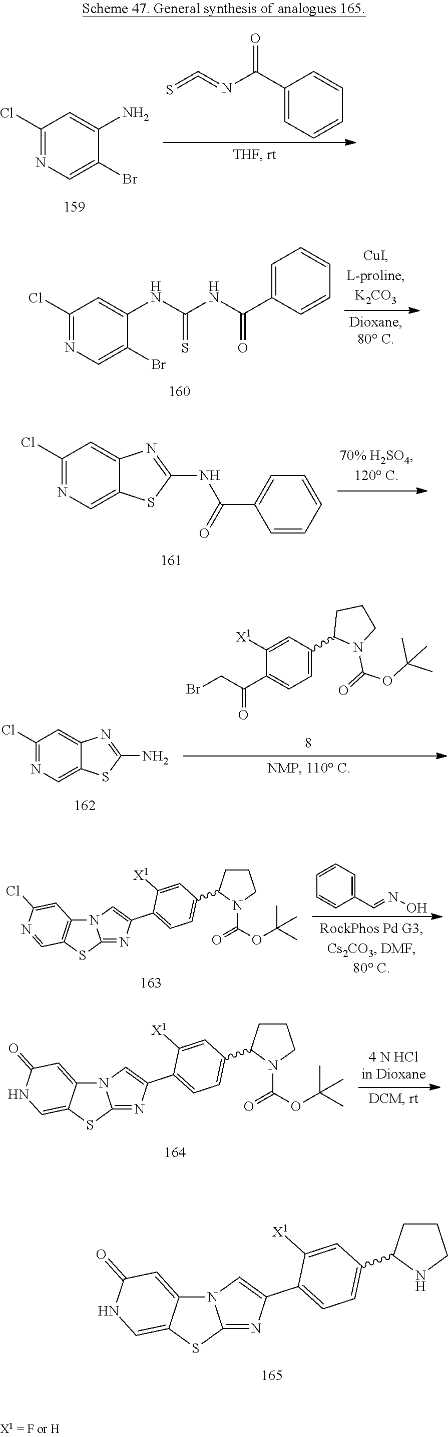

Compounds 163 were synthesized from compound 159 following the same synthetic sequence as in Scheme 45. Pyridone compounds 164 were synthesized from o-chloride pyridines 163 using an alternative benzaldehyde oxime/palladium catalyst system. The N-Boc protecting group was removed to produce final compounds 165 as the free base or as the hydrochloride salt. Chiral resolution was performed on N-Boc protected compounds 164 to generate final enantiomers if required.

### Synthetic Details for Additional Compounds of the Invention

Compounds 72 (Scheme 26) were synthesized from aniline compounds 66 (Scheme 26) following the descriptions outlined in Scheme 21. The ester was hydrolyzed to form carboxylic acids 73. Amide coupling reactions on 73 with various amines gave amides 53. The N-Boc protecting group was removed by 4 N hydrochloride in 1,4-dioxane to generate benzo[d]imidazo[2,1-b]thiazole analogues 54 as a free base or as the hydrochloride salt (Scheme 26).

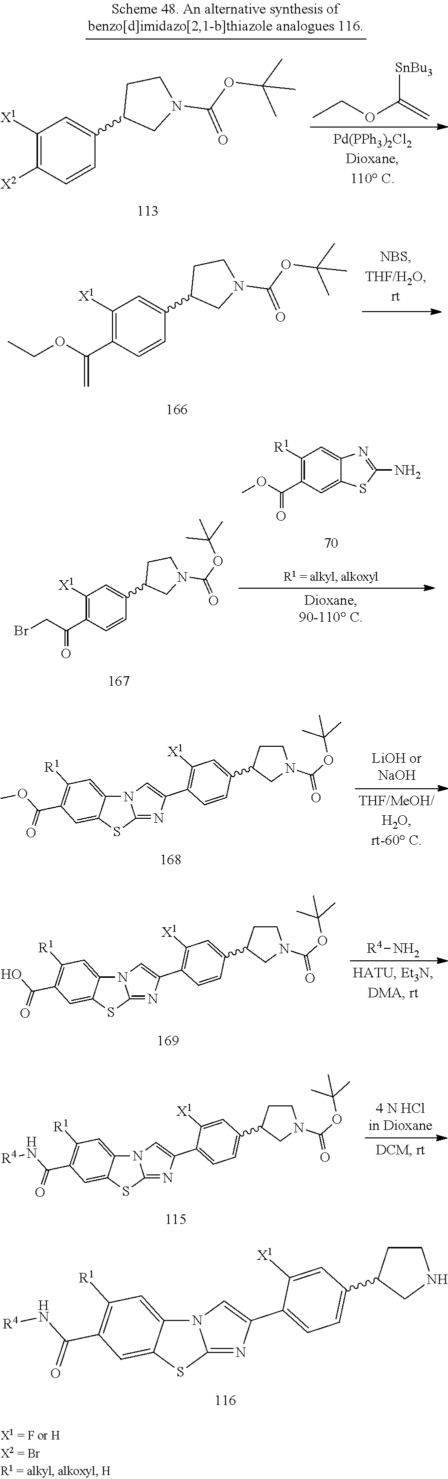

α-bromoketone analogues 167 were synthesized from building blocks 113 following the same synthetic sequence described in Scheme 10. The final compounds 116 were synthesized from α-bromoketone analogues 167 following the same synthetic sequence outlined in Scheme 26. The compounds were isolated as the free base or as the hydrochloride salt.

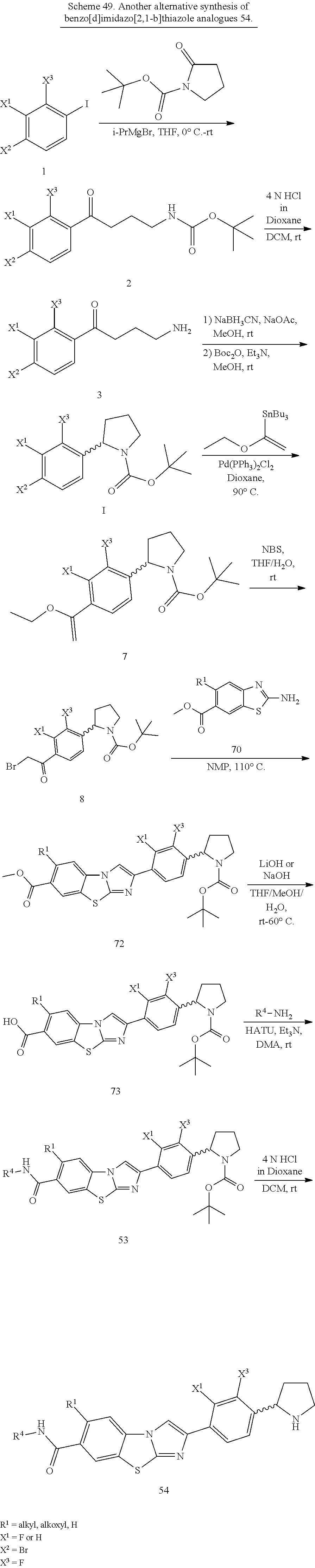

α-bromoketone analogues 8 were synthesized from building blocks 1, following the same synthetic sequence described in Scheme 10. The final compounds 54 were synthesized from α-bromoketone analogues 8 following the same synthetic sequence outlined in Scheme 26. The compounds were isolated as the free base or as the hydrochloride salt.

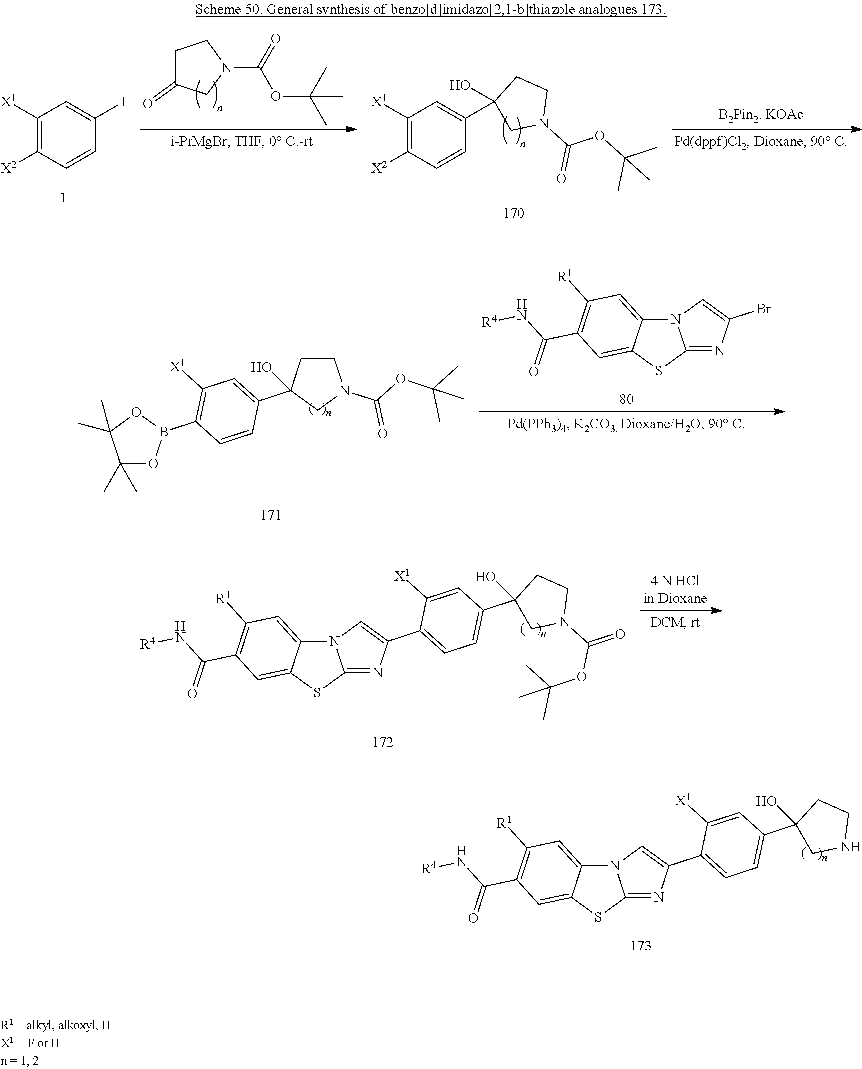

Benzyl alcohol intermediates 170 were synthesized from building blocks 1, following the same synthetic sequence described in Scheme 10. The final compounds 173 were synthesized from intermediates 170 following the same synthetic sequence outlined in Scheme 39. The compounds were isolated as the free base or as the hydrochloride salt.

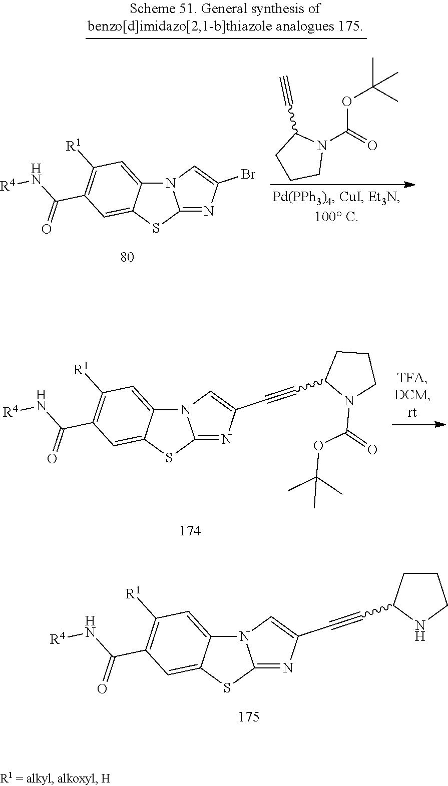

Sonogushira cross-coupling reaction was performed between intermediates 80 and the terminal alkynyl compound shown to provide intermediates 174. The N-Boc protecting group was removed using trifluoroacetic acid in dichloromethane to produce the final benzo[d]imidazo[2,1-b]oxazole analogues 175 as a free base.

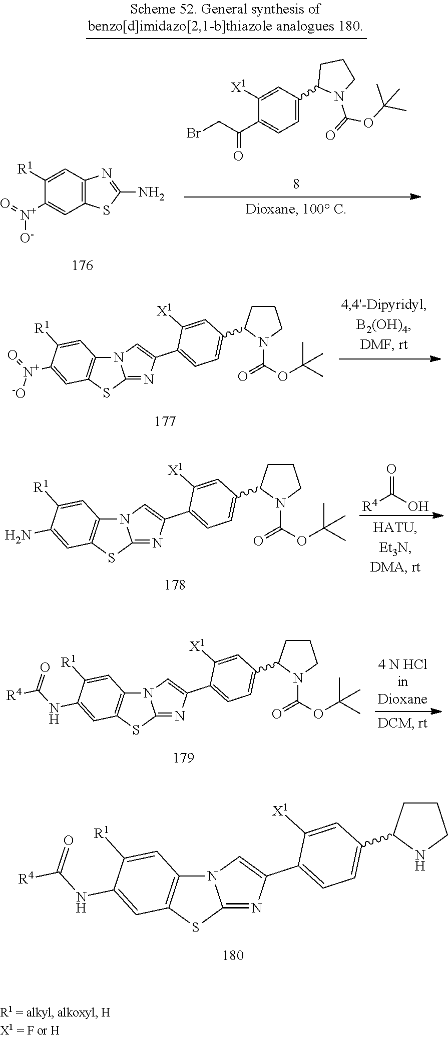

Intermediates 177 were synthesized from 2-aminobenzothiazole building blocks 176 and α-bromoketone analogue 8 at elevated temperatures. Nitro group was reduced by using hypodiboric acid to generate aniline intermediates 178. The final compounds 180 were synthesized from intermediates 178 following the same synthetic sequence described in Scheme 26. The compounds were isolated as the free base or as the hydrochloride salt. Chiral resolution was performed on amide compounds 179 if required.

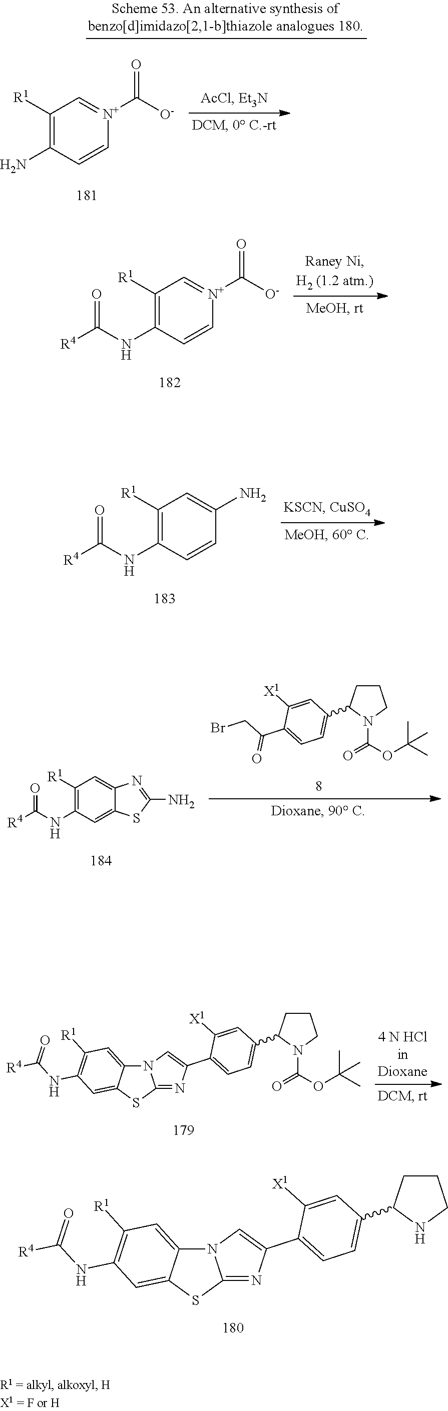

Reaction of anilines 181 with acyl chlorides gave amide intermediates 182. Nitro group was reduced by hydrogenation to produce aniline intermediates 183. The final compounds 180 were synthesized from intermediates 183 following the same synthetic sequence described in Scheme 21. The compounds were isolated as the free base or as the hydrochloride salt. Chiral resolution was performed on amide compounds 179 if required.

### Synthetic Details for Additional Compounds of the Invention

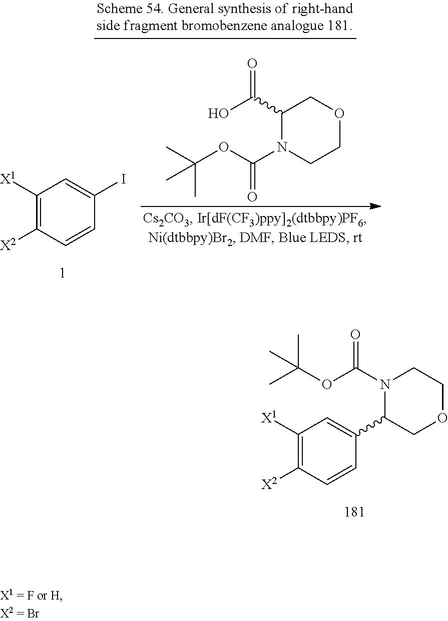

Intermediate 181 was synthesized from starting material 1 and the corresponding commercial available carboxylic acids, 3R/3S-4-(1,1-Dimethylethyl)-3,4-morpholinedicarboxylate using photoredox cross-coupling reaction.

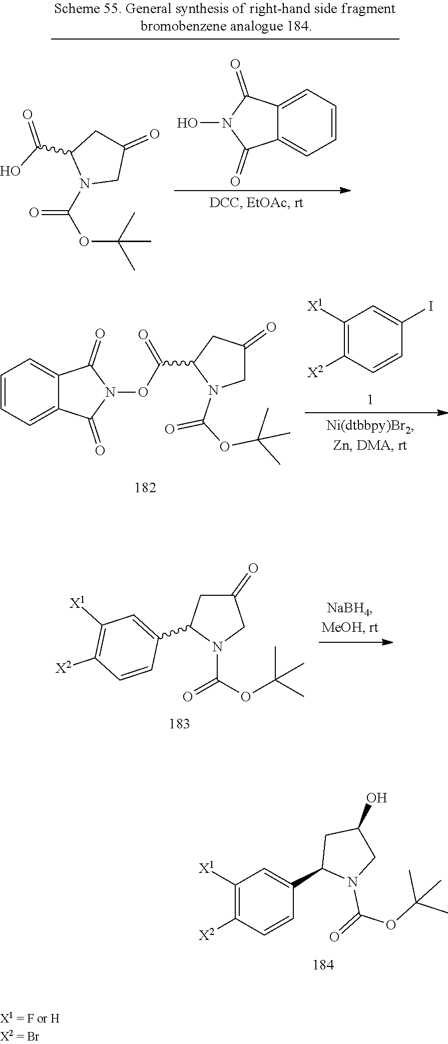

Condensation reaction was performed on commercially available carboxylic acids, 2R/2S-1-(1,1-Dimethylethyl)-4-oxo-1,2-pyrrolidinedicarboxylate with 2-hydroxyisoindoline-1,3-dione to generate activated ester intermediate 182. It was followed by a cross-coupling reaction with compound 1 to generate ketone analogue 183. The ketone 183 was reduced to the cis alcohol intermediate 184.

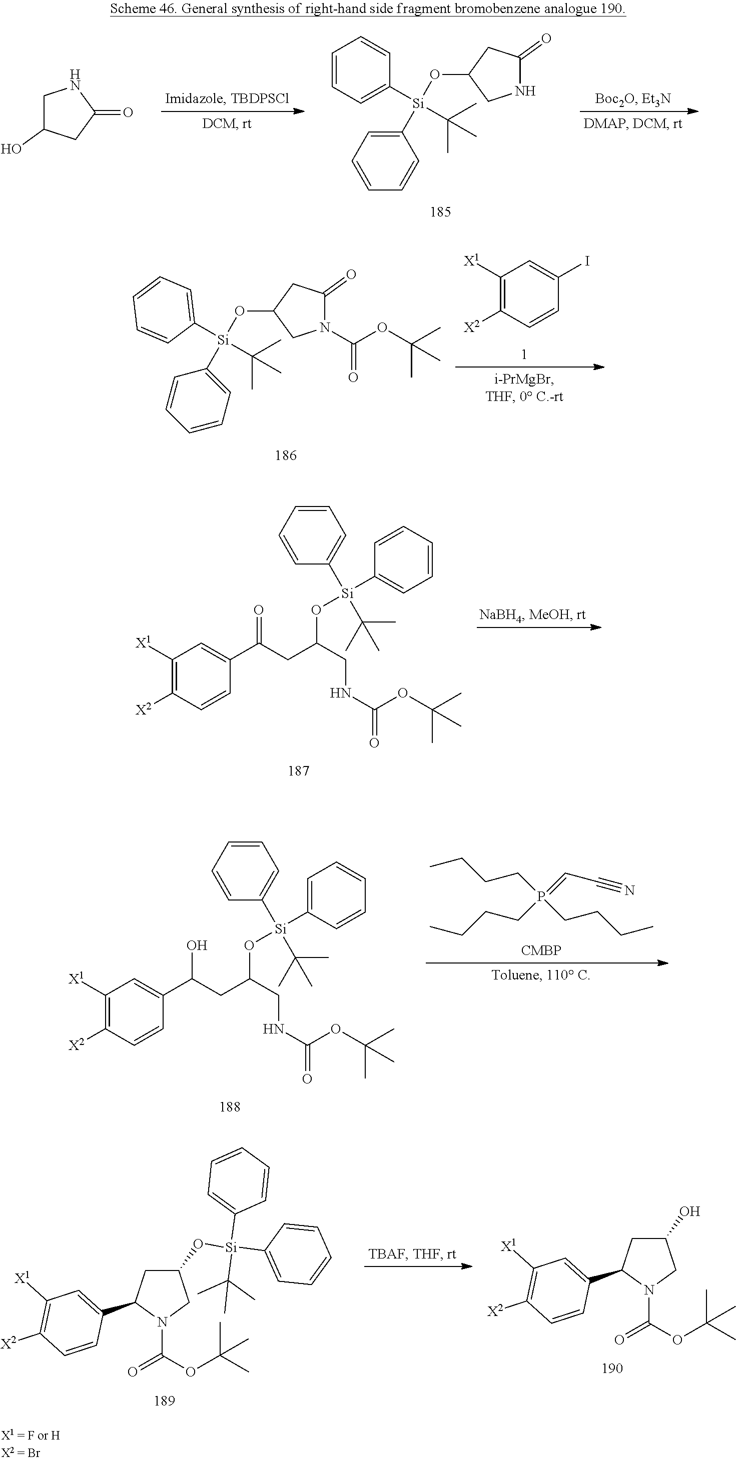

The hydroxyl group of 4-hydroxypyrrolidin-2-one was protected with a TBDPS group to generate intermediate 185. It was followed by Boc protection of the lactam to generate doubly protected intermediate 186. The ketone intermediate 187 was synthesized following the same procedure described in Scheme 10 step 1. The ketone was then reduced to alcohol intermediate 188. A Mitsunobu reaction was performed on 188 using the reagent CMBP to selectively generate trans intermediate 189. The TBDPS group was removed by TBAF to generate intermediate 190.

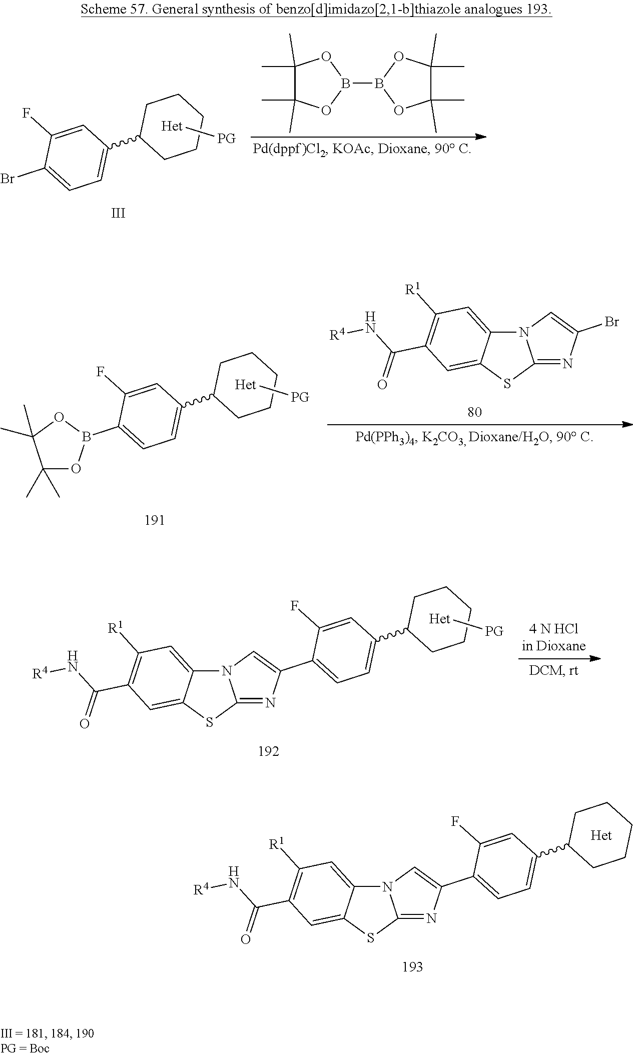

Right-hand side fragment bromobenzene analogues III were converted to their corresponding boronic esters/acids analogues 191 via Miyaura borylation reaction. Cross-coupling reactions with bromides 80 gave an array of protected intermediates 192 possessing differential substitution at the right-hand terminus. The N-Boc protecting group was removed by 4 N hydrochloride in 1,4-dioxane to generate benzo[d]imidazo[2,1-b]thiazole analogues 193 as a free base or as the hydrochloride salt. Chiral resolution was performed on N-Boc protected compounds 192 to generate final enantiomers if required.

### Example 3

### Synthetic Details of Specific Compounds of the Invention (Schemes 58-79)

### Intermediate Preparations

### Synthesis of tert-butyl (S)-2-(3-fluoro-4-(7-((3-(4-fluoropiperidin-1-yl)propyl)carbamoyl)-9-methyl-9H-benzo[d]imidazo[1,2-a]imidazol-2-yl)phenyl)pyrrolidine-1-carboxylate

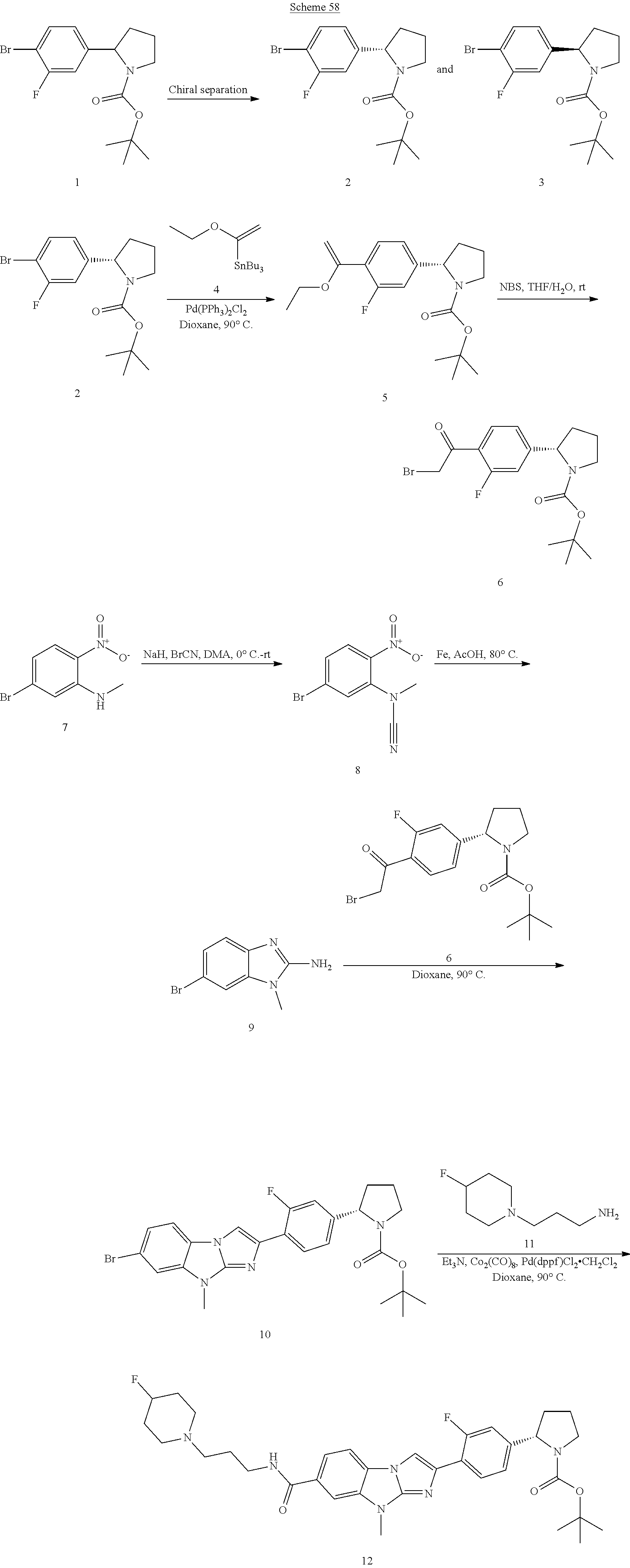

### Synthesis of tert-butyl (S)-2-(4-bromo-3-fluorophenyl)pyrrolidine-1-carboxylate and tert-butyl (R)-2-(4-bromo-3-fluorophenyl)pyrrolidine-1-carboxylate (2, 3 Scheme 58)

Tert-butyl 2-(4-bromo-3-fluorophenyl)pyrrolidine-1-carboxylate (10.46 g, 30.39 mmol) was chirally separated by Prep-SFC with the following conditions; Column: (S,S)-Whelk-01 Kromasil, 3×25 cm, 5 μm; Mobile Phase A: carbon dioxide, Mobile Phase B: methanol (plus 0.1% 2 N ammonia-methanol); Flow rate: 100 mL/min; Gradient: isocratic 20% B in 6 min; Detector: UV 254 nm; The faster eluting peak at 2.7 min was collected and concentrated under reduced pressure to afford tert-butyl (S)-2-(4-bromo-3-fluorophenyl)pyrrolidine-1-carboxylate as a pink solid.

Yield 5.00 g (48%). ¹H NMR (400 MHz, CDCl₃) δ 7.48 (dd, J=7.2, 8.0 Hz, 1H), 6.97 (dd, J=2.0, 9.6 Hz, 1H), 6.88 (dd, J=2.0, 8.0 Hz, 1H), 4.90-4.75 (m, 1H), 3.65-3.50 (m, 2H), 2.40-2.30 (m, 1H), 1.97-1.84 (m, 2H), 1.83-1.74 (m, 1H), 1.49-1.25 (m, 9H). m/z: [ESI+] 344, 346 (M+H)⁺.

The slower eluting peak at 4.35 min was collected and concentrated under reduced pressure to afford tert-butyl (R)-2-(4-bromo-3-fluorophenyl)pyrrolidine-1-carboxylate as a pink solid.

Yield 5.00 g (48%). ¹H NMR (400 MHz, CDCl₃) δ 7.48 (dd, J=7.2, 8.0 Hz, 1H), 6.97 (dd, J=2.0, 9.6 Hz, 1H), 6.88 (dd, J=2.0, 8.0 Hz, 1H), 4.90-4.75 (m, 1H), 3.65-3.50 (m, 2H), 2.40-2.30 (m, 1H), 1.97-1.84 (m, 2H), 1.83-1.74 (m, 1H), 1.49-1.25 (m, 9H). m/z: [ESI⁺] 344, 346 (M+H)⁺.

### Synthesis of tert-butyl (S)-2-(4-(1-ethoxyvinyl)-3-fluorophenyl)pyrrolidine-1-carboxylate (5, Scheme 58)

To a stirred solution of tert-butyl (S)-2-(4-bromo-3-fluorophenyl)pyrrolidine-1-carboxylate (4.50 g, 13.07 mmol) in 1,4-dioxane (80 mL) were added tributyl(1-ethoxyethenyl)stannane (7.12 g, 19.71 mmol) and bis(triphenylphosphine)palladium(II) dichloride (0.90 g, 1.28 mmol) in one portion sequentially at room temperature under a nitrogen atmosphere. The reaction mixture was stirred for 2 h at 90° C. The resulting mixture was cooled to room temperature and diluted with an aqueous solution of saturated sodium bicarbonate (100 mL). The aqueous layer was extracted with ethyl acetate (3×150 mL). The combined organic phase was washed with brine (200 mL) and dried with anhydrous sodium sulfate. After filtration, the filtrate was concentrated under reduced pressure to give tert-butyl (S)-2-(4-(1-ethoxyvinyl)-3-fluorophenyl)pyrrolidine-1-carboxylate as a brown oil. The crude product was used in the next step directly without further purification.

Yield 4.10 g (crude). ¹H NMR not run. m/z: [ESI⁺] 336 (M+H)⁺.

### Synthesis of tert-butyl (S)-2-(4-(2-bromoacetyl)-3-fluorophenyl)pyrrolidine-1-carboxylate (6, Scheme 58)

To a stirred solution of crude tert-butyl (S)-2-(4-(1-ethoxyvinyl)-3-fluorophenyl)pyrrolidine-1-carboxylate (4.10 g) in tetrahydrofuran (45 mL) and water (1 mL) was added N-bromosuccinimide (3.28 g, 18.43 mmol) in one portion at room temperature. The reaction solution was stirred for 1 h at this temperature. The resulting solution was concentrated under reduced pressure. The residue was purified by silica gel column chromatography (0%-20% of ethyl acetate in petroleum ether) to afford tert-butyl (S)-2-(4-(2-bromoacetyl)-3-fluorophenyl)pyrrolidine-1-carboxylate as a light yellow oil.

Yield 4.42 g (88% over two steps). ¹H NMR (400 MHz, CDCl₃) δ 7.91 (dd, J=8.0, 8.0 Hz, 1H), 7.11 (dd, J=1.6, 8.0 Hz, 1H), 7.00 (dd, J=1.6, 12.4 Hz, 1H), 5.00-4.76 (m, 1H), 4.52 (d, J=2.4 Hz, 2H), 3.72-3.50 (m, 2H), 2.46-2.28 (m, 1H), 1.96-1.87 (m, 2H), 1.87-1.77 (m, 1H), 1.47-1.25 (m, 9H). m/z: [ESI⁺] 330, 332 (M+H-56)⁺.

### Synthesis of N-(5-bromo-2-nitrophenyl)-N-methylcyanamide (8, Scheme 58)

To a stirred solution of 5-bromo-N-methyl-2-nitroaniline (2.00 g, 8.66 mmol) in N,N-dimethylacetamide (20 mL) was added sodium hydride (60% dispersion in mineral oil, 0.69 g, 17.25 mmol) portionwise over 3 min at 0° C. under a nitrogen atmosphere. The reaction mixture was stirred for 1 h. To the above solution was added a solution of cyanic bromide (1.83 g, 17.28 mmol) in N,N-dimethylacetamide (1 mL) dropwise over 5 min. The resulting mixture was stirred for additional 2 h at room temperature. The reaction was quenched by an aqueous solution of saturated ammonium chloride (20 mL) at room temperature and extracted with ethyl acetate (3×30 mL). The combined organic layers were washed with water (2×20 mL) and dried over anhydrous sodium sulfate. After filtration, the filtrate was concentrated under reduced pressure. The residue was purified by silica gel column chromatography (1%-50% of ethyl acetate in petroleum ether) to afford N-(5-bromo-2-nitrophenyl)-N-methylcyanamide as a yellow solid.

Yield: 2.00 g (90%). ¹H NMR (300 MHz, CDCl₃) δ 7.88 (d, J=8.7 Hz, 1H), 7.70 (d, J=2.1 Hz, 1H), 7.57 (dd, J=2.1, 8.7 Hz, 1H), 3.37 (s, 3H). m/z: [ESI⁺] 256, 258 (M+H)⁺.

### Synthesis of 6-bromo-1-methyl-1H-benzo[d]imidazol-2-amine (9, Scheme 58)

To a stirred solution of N-(5-bromo-2-nitrophenyl)-N-methylcyanamide (2.00 g, 7.81 mmol) in acetic acid (20 mL) was added iron powder (2.18 g, 39.03 mmol) in one portion at room temperature. The reaction mixture was stirred for 2 h at 80° C. under a nitrogen atmosphere. The mixture was allowed to cool to room temperature and filtered. The filtered cake was washed with ethyl acetate (2×10 mL). The combined filtrates were concentrated under reduced pressure. The residue was diluted with water (40 mL), extracted with ethyl acetate (3×40 mL). The combined organic extracts were dried over anhydrous sodium sulfate. After filtration, the filtrate was concentrated under reduced pressure. The residue was triturated with dichloromethane (20 mL). The precipitated solids were collected by filtration, washed with dichloromethane (2×10 mL) and dried in a vacuum oven to afford 6-bromo-1-methyl-1H-benzo[d]imidazol-2-amine as an off-white solid.

Yield: 1.50 g (85%). ¹H NMR (300 MHz, CDCl₃) δ 7.35 (t, J=1.2 Hz, 1H), 7.07-7.03 (m, 2H), 6.55 (br s, 2H), 3.49 (s, 3H). m/z: [ESI⁺] 226, 228 (M+H)⁺.

### Synthesis of tert-butyl (S)-2-(4-(7-bromo-9-methyl-9H-benzo[d]imidazo[1,2-a]imidazol-2-yl)-3-fluoro phenyl)pyrrolidine-1-carboxylate (10, Scheme 58)

To a stirred solution of 6-bromo-1-methyl-1H-benzo[d]imidazol-2-amine (702 mg, 3.105 mmol) in 1,4-dioxane (10 mL) was added tert-butyl (S)-2-(4-(2-bromoacetyl)-3-fluorophenyl)pyrrolidine-1-carboxylate (600 mg, 1.553 mmol) at room temperature. The reaction solution was stirred for 16 h at 90° C. under a nitrogen atmosphere. The solution was cooled to room temperature and purified directly by reverse phase flash column chromatography with the following conditions; Column: Spherical C18, 20-40 μm, 330 g; Mobile Phase A: water (plus 10 mmol ammonium bicarbonate); Mobile Phase B: acetonitrile; Flow rate: 80 mL/min; Gradient: 65%-85% B in 20 min; Detector: UV 254 nm. The fractions containing desired product were collected at 82% B and concentrated under reduced pressure to afford tert-butyl (S)-2-(4-(7-bromo-9-methyl-9H-benzo[d]imidazo[1,2-a]imidazol-2-yl)-3-fluorophenyl)pyrrolidine-1-carboxylate as a yellow solid.

Yield 600 mg (75%). ¹H NMR (400 MHz, CDCl₃) δ 8.17 (dd, J=8.0, 8.0 Hz, 1H), 7.82 (s, 1H), 7.47-7.41 (m, 2H), 7.34 (dd, J=2.0, 8.4 Hz, 1H), 7.07 (dd, J=1.6, 8.0 Hz, 1H), 6.97 (dd, J=1.6, 12.4 Hz, 1H), 5.03-4.77 (m, 1H), 3.84 (s, 3H), 3.73-3.49 (m, 2H), 2.43-2.26 (m, 1H), 1.99-1.82 (m, 2H), 1.82-1.60 (m, 1H), 1.49-1.25 (m, 9H). m/z: [ESI⁺] 513, 515 (M+H)⁺.

### Synthesis of tert-butyl (S)-2-(3-fluoro-4-(7-((3-(4-fluoropiperidin-1-yl)propyl)carbamoyl)-9-methyl-9H-benzo[d]imidazo[1,2-a]imidazol-2-yl)phenyl)pyrrolidine-1-carboxylate (12, Scheme 58)

To a stirred solution of tert-butyl (S)-2-(4-(7-bromo-9-methyl-9H-benzo[d]imidazo[1,2-a]imidazol-2-yl)-3-fluorophenyl)pyrrolidine-1-carboxylate (300 mg, 0.584 mmol) and 3-(4-fluoropiperidin-1-yl)propan-1-amine (187 mg, 1.167 mmol) in 1,4-dioxane (10 mL) were added triethylamine (177 mg, 1.749 mmol), 1,1′-bis(diphenylphosphino)ferrocene-palladium(II)dichloride dichloromethane complex (48 mg, 0.059 mmol) and dicobalt octacarbonyl (60 mg, 0.175 mmol) at room temperature under a nitrogen atmosphere. The reaction mixture was stirred for 16 h at 90° C. The mixture was cooled to room temperature, diluted with water (10 mL) and extracted with ethyl acetate (3×20 mL). The combined organic layers were washed with water (3×20 mL) and dried over anhydrous sodium sulfate. After filtration, the filtrate was concentrated under reduced pressure. The residue was purified by silica gel column chromatography (10% of methanol in dichloromethane) to afford tert-butyl (S)-2-(3-fluoro-4-(7-((3-(4-fluoropiperidin-1-yl)propyl)carbamoyl)-9-methyl-9H-benzo[d]imidazo[1,2-a]imidazol-2-yl)phenyl)pyrrolidine-1-carboxylate as a brown oil.

Yield 200 mg (55%). ¹H NMR (400 MHz, CDCl₃) δ 8.31-8.22 (m, 1H), 8.20-8.12 (m, 1H), 8.01-7.92 (m, 1H), 7.89-7.80 (m, 1H), 7.71-7.61 (m, 1H), 7.61-7.52 (m, 1H), 7.11-7.03 (m, 1H), 6.99-6.94 (m, 1H), 5.03-4.66 (m, 2H), 3.90 (s, 3H), 3.71-3.60 (m, 4H), 2.86-2.50 (m, 6H), 2.41-2.26 (m, 1H), 2.03-1.81 (m, 9H), 1.50-1.25 (m, 9H). m/z: [ESI⁺] 621 (M+H)⁺.

### Synthesis of Tert-butyl (R)-2-(3-fluoro-4-(7-((3-(4-fluoropiperidin-1-yl)propyl)carbamoyl)-9-methyl-9H-benzo[d]imidazo[1,2-a]imidazol-2-yl)phenyl)pyrrolidine-1-carboxylate

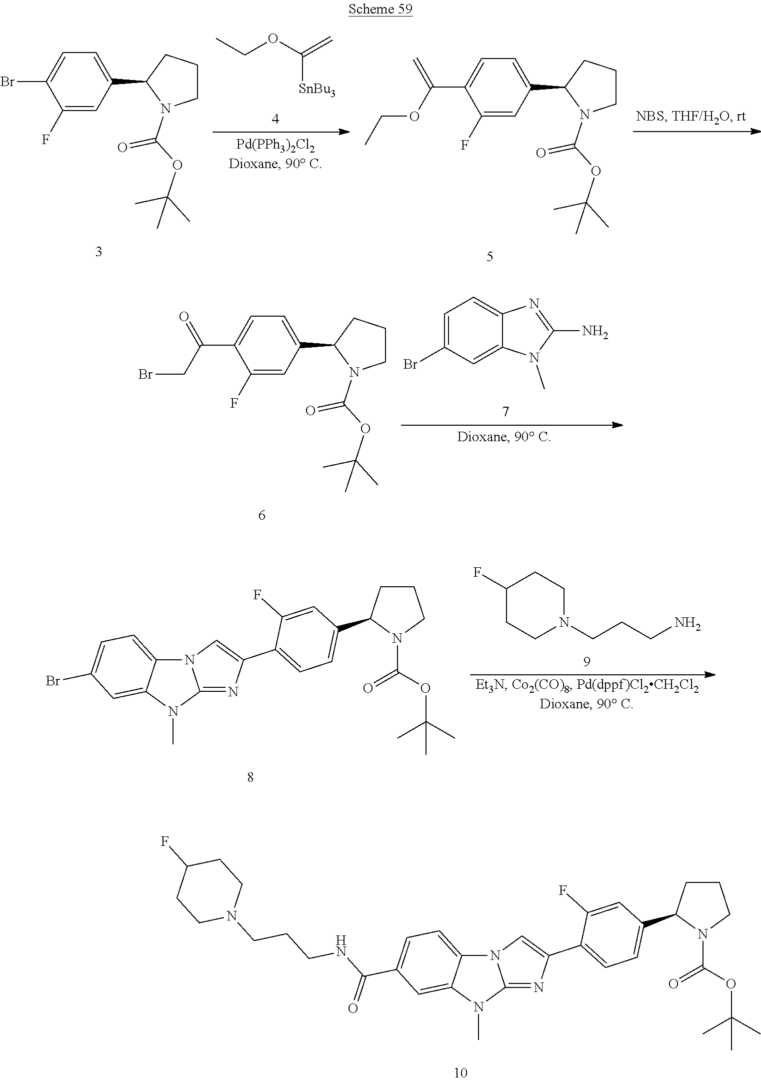

### Synthesis of tert-butyl (R)-2-(4-(1-ethoxyvinyl)-3-fluorophenyl)pyrrolidine-1-carboxylate (5, Scheme 59)

Compound tert-butyl (R)-2-(4-(1-ethoxyvinyl)-3-fluorophenyl)pyrrolidine-1-carboxylate was prepared from tert-butyl (R)-2-(4-bromo-3-fluorophenyl)pyrrolidine-1-carboxylate (2.35 g, 6.83 mmol) and tributyl(1-ethoxyethenyl)stannane (3.70 g, 10.24 mmol) following a similar procedure to that described for the synthesis of tert-butyl (S)-2-(4-(1-ethoxyvinyl)-3-fluorophenyl)pyrrolidine-1-carboxylate, and was extracted and concentrated under reduced pressure as a brown oil.

Yield 2.00 g (crude). ¹H NMR not run. m/z: [ESI⁺] 336 (M+H)⁺.

### Synthesis of tert-butyl (R)-2-(4-(2-bromoacetyl)-3-fluorophenyl)pyrrolidine-1-carboxylate (6, Scheme 59)

Compound tert-butyl (R)-2-(4-(2-bromoacetyl)-3-fluorophenyl)pyrrolidine-1-carboxylate was prepared from tert-butyl (R)-2-(4-(1-ethoxyvinyl)-3-fluorophenyl)pyrrolidine-1-carboxylate (2.00 g, crude) and N-bromosuccinimide (1.70 g, 9.55 mmol) following a similar procedure to that described for the synthesis of tert-butyl (S)-2-(4-(2-bromoacetyl)-3-fluorophenyl)pyrrolidine-1-carboxylate, and was isolated as a light yellow oil.

Yield 2.21 g (84% over two steps). ¹H NMR (400 MHz, CDCl₃) δ 7.91 (dd, J=8.0, 8.0 Hz, 1H), 7.11 (dd, J=1.6, 8.0 Hz, 1H), 7.00 (dd, J=1.6, 12.4 Hz, 1H), 5.00-4.76 (m, 1H), 4.52 (d, J=2.4 Hz, 2H), 3.72-3.50 (m, 2H), 2.46-2.28 (m, 1H), 1.96-1.87 (m, 2H), 1.87-1.77 (m, 1H), 1.47-1.25 (m, 9H). m/z: [ESI⁺] 330, 332 (M+H-56)⁺.

### Synthesis of tert-butyl (R)-2-(4-(7-bromo-9-methyl-9H-benzo[d]imidazo[1,2-a]imidazol-2-yl)-3-fluoro phenyl)pyrrolidine-1-carboxylate (8, Scheme 59)

Compound tert-butyl (R)-2-(4-(7-bromo-9-methyl-9H-benzo[d]imidazo[1,2-a]imidazol-2-yl)-3-fluorophenyl)pyrrolidine-1-carboxylate was prepared from 6-bromo-1-methyl-1H-benzo[d]imidazol-2-amine (0.70 g, 3.10 mmol) and tert-butyl (R)-2-(4-(2-bromoacetyl)-3-fluorophenyl)pyrrolidine-1-carboxylate (0.60 g, 1.55 mmol) following a similar procedure to that described for the synthesis of tert-butyl (S)-2-(4-(7-bromo-9-methyl-9H-benzo[d]imidazo[1,2-a]imidazol-2-yl)-3-fluorophenyl)pyrrolidine-1-carboxylate, and was isolated as a yellow solid.

Yield 0.65 g (82%). ¹H NMR (400 MHz, CDCl₃) δ 8.17 (dd, J=8.0, 8.0 Hz, 1H), 7.82 (s, 1H), 7.47-7.41 (m, 2H), 7.34 (dd, J=2.0, 8.4 Hz, 1H), 7.07 (dd, J=1.6, 8.0 Hz, 1H), 6.97 (dd, J=1.6, 12.4 Hz, 1H), 5.03-4.77 (m, 1H), 3.84 (s, 3H), 3.73-3.49 (m, 2H), 2.43-2.26 (m, 1H), 1.99-1.82 (m, 2H), 1.82-1.60 (m, 1H), 1.49-1.25 (m, 9H). m/z: [ESI⁺] 513, 515 (M+H)⁺.

### Synthesis of tert-butyl (R)-2-(3-fluoro-4-(7-((3-(4-fluoropiperidin-1-yl)propyl)carbamoyl)-9-methyl-9H-benzo[d]imidazo[1,2-a]imidazol-2-yl)phenyl)pyrrolidine-1-carboxylate (10, Scheme 59)

Compound tert-butyl (R)-2-(3-fluoro-4-(7-((3-(4-fluoropiperidin-1-yl)propyl)carbamoyl)-9-methyl-9H-benzo[d]imidazo[1,2-a]imidazol-2-yl)phenyl)pyrrolidine-1-carboxylate was prepared from tert-butyl (R)-2-(4-(7-bromo-9-methyl-9H-benzo[d]imidazo[1,2-a]imidazol-2-yl)-3-fluorophenyl)pyrrolidine-1-carboxylate (0.30 g, 0.58 mmol) and 3-(4-fluoropiperidin-1-yl)propan-1-amine (0.19 g, 1.18 mmol) following a similar procedure to that described for the synthesis of tert-butyl (S)-2-(3-fluoro-4-(7-((3-(4-fluoropiperidin-1-yl)propyl)carbamoyl)-9-methyl-9H-benzo[d]imidazo[1,2-a]imidazol-2-yl)phenyl)pyrrolidine-1-carboxylate, and was isolated as a brown oil.

Yield 0.20 g (55%). ¹H NMR (400 MHz, CDCl₃) δ 8.31-8.22 (m, 1H), 8.20-8.12 (m, 1H), 8.01-7.92 (m, 1H), 7.89-7.80 (m, 1H), 7.71-7.61 (m, 1H), 7.61-7.52 (m, 1H), 7.11-7.03 (m, 1H), 6.99-6.94 (m, 1H), 5.03-4.66 (m, 2H), 3.90 (s, 3H), 3.71-3.60 (m, 4H), 2.86-2.50 (m, 6H), 2.41-2.26 (m, 1H), 2.03-1.81 (m, 9H), 1.50-1.25 (m, 9H). m/z: [ESI⁺] 621 (M+H)⁺.

### Synthesis of tert-butyl 6-fluoro-5-(7-((3-(4-fluoropiperidin-1-yl)propyl)carbamoyl)benzo[d]imidazo[2, 1-b]thiazol-2-yl)indoline-1-carboxylate

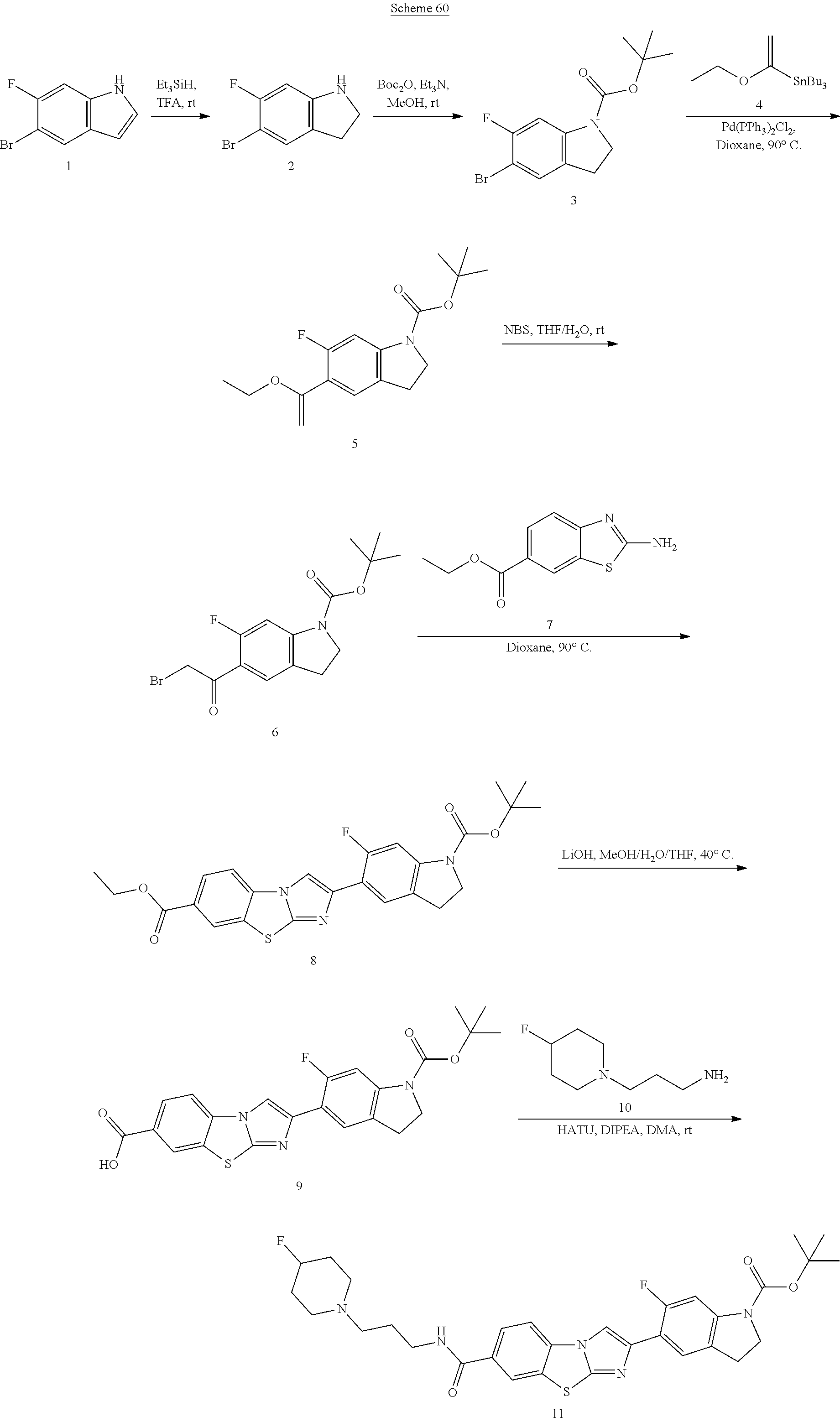

### Synthesis of 5-bromo-6-fluoroindoline Trifluoroacetic Acid (2, Scheme 60)

A solution of 5-bromo-6-fluoro-1H-indole (1.00 g, 4.67 mmol) and triethylsilane (1.63 g, 14.02 mmol) in trifluoroacetic acid (10 mL) was stirred for 2 h at room temperature under a nitrogen atmosphere. The resulting solution was concentrated under reduced pressure to afford 5-bromo-6-fluoroindoline trifluoroacetic acid as a brown oil, which was used in the next step directly without further purification.

Yield 1.50 g (crude). ¹H NMR not run. m/z: [ESI⁺] 216, 218 (M+H)⁺.

### Synthesis of tert-butyl 5-bromo-6-fluoroindoline-1-carboxylate (3, Scheme 60)

To a stirred solution of the above crude 5-bromo-6-fluoroindoline trifluoroacetic acid (1.50 g) and triethylamine (1.38 g, 13.64 mmol) in dichloromethane (15 mL) was added di-tert-butyl dicarbonate (2.98 g, 13.65 mmol) in one portion at room temperature under a nitrogen atmosphere. The resulting mixture was stirred for 2 h. then concentrated under reduced pressure. The residue was purified by silica gel column chromatography (30% of ethyl acetate in petroleum ether) to afford tert-butyl 6-bromo-5-fluoroindoline-1-carboxylate as a light yellow oil.

Yield 0.40 g (27% over two steps). ¹H NMR (400 MHz, CDCl₃) δ 7.71 (s, 1H), 7.26 (d, J=6.8 Hz, 1H), 4.03 (t, J=8.8 Hz, 2H), 3.07 (t, J=8.8 Hz, 2H), 1.57 (s, 9H). LCMS mass signal not determined.

### Synthesis of tert-butyl 5-(1-ethoxyvinyl)-6-fluoroindoline-1-carboxylate (5, Scheme 60)

Compound tert-butyl 5-(1-ethoxyvinyl)-6-fluoroindoline-1-carboxylate was prepared from tert-butyl 5-bromo-6-fluoroindoline-1-carboxylate (400 mg, 1.265 mmol) and tributyl(1-ethoxyvinyl)stannane (685 mg, 1.897 mmol) following a similar procedure to that described for the synthesis of tert-butyl (S)-2-(4-(1-ethoxyvinyl)-3-fluorophenyl)pyrrolidine-1-carboxylate, and was extracted and concentrated under reduced pressure as a yellow solid.

Yield 400 mg (crude). ¹H NMR not run. m/z: [ESI⁺] 308 (M+H)⁺.

### Synthesis of tert-butyl 5-(2-bromoacetyl)-6-fluoroindoline-1-carboxylate (6, Scheme 60)

Compound tert-butyl 5-(2-bromoacetyl)-6-fluoroindoline-1-carboxylate was prepared from tert-butyl 5-(1-ethoxyvinyl)-6-fluoroindoline-1-carboxylate (400 mg, crude) following a similar procedure to that described for the synthesis of tert-butyl (S)-2-(4-(2-bromoacetyl)-3-fluorophenyl)pyrrolidine-1-carboxylate, and was isolated as a light brown solid.

Yield 200 mg (44% over two steps). ¹H NMR (400 MHz, CDCl₃) δ 7.73 (d, J=7.2 Hz, 1H), 7.67 (s, 1H), 4.51 (d, J=2.8 Hz, 2H), 4.08 (t, J=8.8 Hz, 2H), 3.11 (t, J=8.8 Hz, 2H), 1.60 (s, 9H). m/z: [ESI⁺]358, 360 (M+H)⁺.

### Synthesis of ethyl 2-(1-(tert-butoxycarbonyl)-6-fluoroindolin-5-yl)benzo[d]imidazo[2,1-b]thiazole-7-carboxylate (8, Scheme 60)

Compound ethyl 2-(1-(tert-butoxycarbonyl)-6-fluoroindolin-5-yl)benzo[d]imidazo[2,1-b]thiazole-7-carboxylate was prepared from tert-butyl 5-(2-bromoacetyl)-6-fluoroindoline-1-carboxylate (200 mg, 0.558 mmol) and ethyl 2-aminobenzo[d]thiazole-6-carboxylate (124 mg, 0.558 mmol) following a similar procedure to that described for the synthesis of tert-butyl (S)-2-(4-(7-bromo-9-methyl-9H-benzo[d]imidazo[1,2-a]imidazol-2-yl)-3-fluorophenyl)pyrrolidine-1-carboxylate, and was isolated as a light brown solid.

Yield 140 mg (52%). ¹H NMR (400 MHz, DMSO) δ 8.68 (d, J=1.6 Hz, 1H), 8.59 (d, J=3.6 Hz, 1H), 8.25 (d, J=8.4 Hz, 1H), 8.10 (dd, J=1.6, 8.4 Hz, 1H), 7.91 (d, J=7.6 Hz, 1H), 7.61-7.48 (m, 1H), 4.37 (q, J=7.2 Hz, 2H), 3.98 (t, J=8.8 Hz, 2H), 3.11 (t, J=8.8 Hz, 2H), 1.53 (s, 9H), 1.37 (t, J=7.2 Hz, 3H). m/z: [ESI⁺] 482 (M+H)⁺.

### Synthesis of 2-(1-(tert-butoxycarbonyl)-6-fluoroindolin-5-yl)benzo[d]imidazo[2,1-b]thiazole-7-carboxylic Acid (9, Scheme 60)

A solution of ethyl 2-(1-(tert-butoxycarbonyl)-6-fluoroindolin-5-yl)benzo[d]imidazo[2,1-b]thiazole-7-carboxylate (140 mg, 0.291 mmol) and lithium hydroxide (35 mg, 1.461 mmol) in tetrahydrofuran:methanol:water (4:4:1, 4.5 mL) was stirred for 16 h at 40° C. The resulting mixture was concentrated under reduced pressure. The residue was acidified to pH 6 with an aqueous solution of 0.5 N hydrochloric acid. The precipitated solids were collected by filtration, washed with acetonitrile (2×5 mL) and dried under reduced pressure to give 2-(1-(tert-butoxycarbonyl)-6-fluoroindolin-5-yl)benzo[d]imidazo[2,1-b]thiazole-7-carboxylic acid as a white solid.

Yield 120 mg (91%). ¹H NMR (400 MHz, DMSO) δ 13.18 (br s, 1H), 8.65 (d, J=1.6 Hz, 1H), 8.60 (d, J=3.6 Hz, 1H), 8.24 (d, J=8.4 Hz, 1H), 8.10 (dd, J=1.6, 8.4 Hz, 1H), 7.93 (d, J=7.6 Hz, 1H), 7.61-7.47 (m, 1H), 3.99 (t, J=8.8 Hz, 2H), 3.11 (t, J=8.8 Hz, 2H), 1.54 (s, 9H). m/z: [ESI⁺] 454 (M+H)⁺.

### Synthesis of tert-butyl 6-fluoro-5-(7-((3-(4-fluoropiperidin-1-yl)propyl)carbamoyl)benzo[d]imidazo[2, 1-b]thiazol-2-yl)indoline-1-carboxylate (11, Scheme 60)

To a stirred solution of 2-(1-(tert-butoxycarbonyl)-6-fluoroindolin-5-yl)benzo[d]imidazo[2,1-b]thiazole-7-carboxylic acid (120 mg, 0.265 mmol) and O-(7-azabenzotriazol-1-yl)-N,N,N′,N′-tetramethyluronium hexafluorophosphate (HATU) (151 mg, 0.397 mmol) in N,N-dimethylacetamide (5 mL) were added N-ethyl-N-isopropylpropan-2-amine (205 mg, 1.586 mmol) and 3-(4-fluoropiperidin-1-yl)propan-1-amine (127 mg, 0.793 mmol) in one portion sequentially at room temperature under a nitrogen atmosphere. The resulting solution was stirred for 2 h. The reaction mixture was then concentrated under reduced pressure. The residue was purified by reverse phase flash column chromatography with the following conditions; column, C18 silica gel, 20-40 μm, 330 g; Mobile phase A: water (plus 10 mmol/L ammonium bicarbonate); Mobile phase B: acetonitrile: Flow rate: 80 mL/min; Gradient: 55%-75% B in 20 min; Detector, UV 254 nm. The fractions containing desired product were collected at 68% B and concentrated under reduced pressure to afford tert-butyl 6-fluoro-5-(7-((3-(4-fluoropiperidin-1-yl)propyl)carbamoyl)benzo[d]imidazo[2,1-b]thiazol-2-yl)indoline-1-carboxylate as a white solid.

Yield 80 mg (51%). ¹H NMR (400 MHz, DMSO) δ 8.64-8.56 (m, 2H), 8.48 (d, J=1.6 Hz, 1H), 8.23 (dd, J=1.6, 8.4 Hz, 1H), 8.00 (dd, J=1.6, 8.4 Hz, 1H), 7.93 (d, J=7.6 Hz, 1H), 7.64-7.48 (m, 1H), 4.67 (tdd, J=3.6, 7.2, 49.2 Hz, 1H), 3.99 (t, J=8.8 Hz, 2H), 3.33-3.32 (m, 2H), 3.12 (t, J=8.8 Hz, 2H), 2.57-2.52 (m, 2H), 2.42-2.33 (m, 2H), 2.32-2.22 (m, 2H), 1.94-1.77 (m, 2H), 1.76-1.63 (m, 4H), 1.54 (s, 9H). m/z: [ESI⁺] 596 (M+H)⁺.

### Synthesis of tert-butyl 6-fluoro-7-(7-((3-(4-fluoropiperidin-1-yl)propyl)carbamoyl)benzo[d]imidazo[2,1-b]thiazol-2-yl)-3,4-dihydroisoquinoline-2(1H)-carboxylate

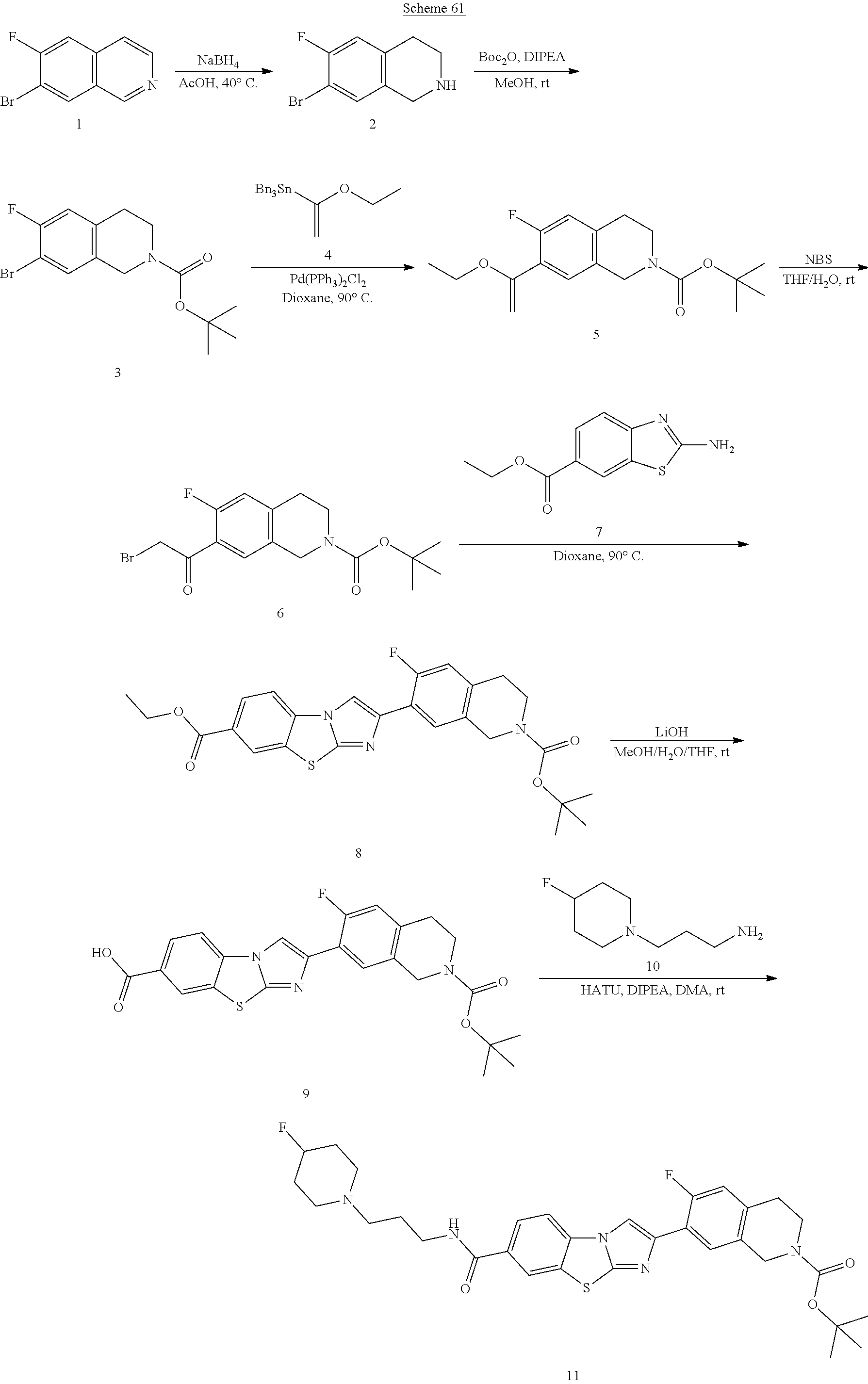

### Synthesis of 7-bromo-6-fluoro-1,2,3,4-tetrahydroisoquinoline (2, Scheme 61)

A solution of 7-bromo-6-fluoroisoquinoline (1.00 g, 4.42 mmol) and sodium borohydride (0.50 g, 13.22 mmol) in acetic acid (5 mL) was stirred for 3 h at 40° C. The resulting mixture was allowed to cool to room temperature and concentrated under reduced pressure to afford 7-bromo-6-fluoro-1,2,3,4-tetrahydroisoquinoline as a colorless oil.

Yield 1.50 g (crude). ¹H NMR (400 MHz, CDCl₃) δ 7.33 (d, J=6.8 Hz, 1H), 6.96 (d, J=8.8 Hz, 1H), 4.26 (s, 2H), 3.42 (t, J=6.4 Hz, 2H), 3.03 (t, J=6.4 Hz, 2H). NH proton not observed. m/z: [ESI⁺] 230, 232 (M+H)⁺.

### Synthesis of tert-butyl 7-bromo-6-fluoro-3,4-dihydroisoquinoline-2(1H)-carboxylate (3, Scheme 61)

Compound tert-butyl 7-bromo-6-fluoro-3,4-dihydroisoquinoline-2(1H)-carboxylate was prepared from 7-bromo-6-fluoro-1,2,3,4-tetrahydroisoquinoline (1.50 g, crude) following a similar procedure to that described for the synthesis of tert-butyl 6-bromo-5-fluoroindoline-1-carboxylate, and was isolated as a light yellow solid.

Yield 1.06 g (73% over two steps). ¹H NMR (300 MHz, CDCl₃) δ 7.31 (d, J=6.9 Hz, 1H), 6.92 (d, J=8.7 Hz, 1H), 4.53 (s, 2H), 3.64 (t, J=6.0 Hz, 2H), 2.80 (t, J=6.0 Hz, 2H), 1.51 (s, 9H). m/z: [ESI⁺]274, 276 (M+H-56)⁺.

### Synthesis of tert-butyl 7-(1-ethoxyvinyl)-6-fluoro-3, 4-dihydroisoquinoline-2(1H)-carboxylate (5, Scheme 61)

Compound tert-butyl 7-(1-ethoxyvinyl)-6-fluoro-3,4-dihydroisoquinoline-2(1H)-carboxylate was prepared from tert-butyl 7-bromo-6-fluoro-3,4-dihydroisoquinoline-2(1H)-carboxylate (1.06 g, 3.21 mmol) following a similar procedure to that described for the synthesis of tert-butyl (S)-2-(4-(1-ethoxyvinyl)-3-fluorophenyl)pyrrolidine-1-carboxylate, and was extracted and concentrated under reduced pressure as a brown oil.

Yield 1.30 g (crude). ¹H NMR not run. m/z: [ESI⁺] 322 (M+H)⁺.

### Synthesis of tert-butyl 7-(2-bromoacetyl)-6-fluoro-3,4-dihydroisoquinoline-2(1H)-carboxylate (6, Scheme 61)

Compound tert-butyl 7-(2-bromoacetyl)-6-fluoro-3,4-dihydroisoquinoline-2(1H)-carboxylate was prepared from tert-butyl 7-(1-ethoxyvinyl)-6-fluoro-3,4-dihydroisoquinoline-2(1H)-carboxylate (1.30 g, crude) following a similar procedure to that described for the synthesis of tert-butyl (S)-2-(4-(2-bromoacetyl)-3-fluorophenyl)pyrrolidine-1-carboxylate, and was isolated as a white solid.

Yield 1.10 g (92% over two steps). ¹H NMR (400 MHz, CDCl₃) δ 7.71 (d, J=7.2 Hz, 1H), 6.98 (d, J=11.6 Hz, 1H), 4.60 (s, 2H), 4.51 (d, J=2.4 Hz, 2H), 3.67 (t, J=6.0 Hz, 2H), 2.90 (t, J=6.0 Hz, 2H), 1.51 (s, 9H). m/z: [ESI⁺] 316, 318 (M+H-56)⁺.

### Synthesis of ethyl 2-(2-(tert-butoxycarbonyl)-6-fluoro-1,2,3,4-tetrahydroisoquinolin-7-yl)benzo[d]imidazo[2,1-b]thiazole-7-carboxylate (8, Scheme 61)

Compound ethyl 2-(2-(tert-butoxycarbonyl)-6-fluoro-1,2,3,4-tetrahydroisoquinolin-7-yl)benzo[d]imidazo[2,1-b]thiazole-7-carboxylate was prepared from tert-butyl 7-(2-bromoacetyl)-6-fluoro-3,4-dihydroisoquinoline-2(1H)-carboxylate (0.70 g, 1.88 mmol) and ethyl 2-aminobenzo[d]thiazole-6-carboxylate (0.42 g, 1.89 mmol) following a similar procedure to that described for the synthesis of tert-butyl (S)-2-(4-(7-bromo-9-methyl-9H-benzo[d]imidazo[1,2-a]imidazol-2-yl)-3-fluorophenyl)pyrrolidine-1-carboxylate, and was isolated as a brown solid.

Yield 0.80 g (86%). ¹H NMR (300 MHz, CDCl₃) δ 8.48 (d, J=1.5 Hz, 1H), 8.23 (dd, J=1.5, 8.4 Hz, 1H), 8.19 (d, J=3.6 Hz, 1H), 7.99 (d, J=7.5 Hz, 1H), 7.74 (d, J=8.4 Hz, 1H), 7.00 (s, 1H), 4.66 (s, 2H), 4.47 (q, J=7.2 Hz, 2H), 3.70 (t, J=6.0 Hz, 2H), 2.88 (t, J=6.0 Hz, 2H), 1.52 (s, 9H), 1.28 (t, J=7.2 Hz, 3H). m/z: [ESI⁺] 496 (M+H)⁺.

### Synthesis of 2-(2-(tert-butoxycarbonyl)-6-fluoro-1,2,3,4-tetrahydroisoquinolin-7-yl)benzo[d]imidazo[2,1-b]thiazole-7-carboxylic Acid (9, Scheme 61)

Compound 2-(2-(tert-butoxycarbonyl)-6-fluoro-1,2,3,4-tetrahydroisoquinolin-7-yl)benzo[d]imidazo[2,1-b]thiazole-7-carboxylic acid was prepared from ethyl 2-(2-(tert-butoxycarbonyl)-6-fluoro-1,2,3,4-tetrahydroisoquinolin-7-yl)benzo[d]imidazo[2,1-b]thiazole-7-carboxylate (0.80 g, 1.61 mmol) following a similar procedure to that described for the synthesis of 2-(1-(tert-butoxycarbonyl)-5-fluoroindolin-6-yl)benzo[d]imidazo[2,1-b]thiazole-7-carboxylic acid, and was isolated as a brown solid.

Yield 0.70 g (93%). ¹H NMR (400 MHz, DMSO) δ 8.70 (d, J=3.6 Hz, 1H), 8.67 (d, J=1.6 Hz, 1H), 8.27 (d, J=8.4 Hz, 1H), 8.10 (dd, J=1.6, 8.4 Hz, 1H), 7.92 (d, J=7.6 Hz, 1H), 7.17 (d, J=12.0 Hz, 1H), 4.56 (s, 2H), 3.57 (t, J=6.0 Hz, 2H), 2.82 (t, J=6.0 Hz, 2H), 1.44 (s, 9H). OH proton not observed. m/z: [ESI⁺] 468 (M+H)⁺.

### Synthesis of tert-butyl 6-fluoro-7-(7-((3-(4-fluoropiperidin-1-yl)propyl)carbamoyl)benzo[d]imidazo[2,1-b]thiazol-2-yl)-3,4-dihydroisoquinoline-2(1H)-carboxylate (11, Scheme 61)

Compound tert-butyl 6-fluoro-7-(7-((3-(4-fluoropiperidin-1-yl)propyl)carbamoyl)benzo[d]imidazo[2,1-b]thiazol-2-yl)-3,4-dihydroisoquinoline-2(1H)-carboxylate was prepared from 2-(2-(tert-butoxycarbonyl)-6-fluoro-1,2,3,4-tetrahydroisoquinolin-7-yl)benzo[d]imidazo[2,1-b]thiazole-7-carboxylic acid (0.70 g, 1.50 mmol) and 3-(4-fluoropiperidin-1-yl)propan-1-amine (0.29 g, 1.81 mmol) following a similar procedure to that described for the synthesis of tert-butyl 5-fluoro-6-(7-((3-(4-fluoropiperidin-1-yl)propyl)carbamoyl)benzo[d]imidazo[2,1-b]thiazol-2-yl)indoline-1-carboxylate, and was isolated as a reddish brown solid.

Yield 0.60 g (66%). ¹H NMR (400 MHz, DMSO) δ 8.68 (d, J=3.6 Hz, 1H), 8.65 (t, J=5.6 Hz, 1H), 8.50 (d, J=1.6 Hz, 1H), 8.25 (d, J=8.4 Hz, 1H), 8.01 (dd, J=1.6, 8.4 Hz, 1H), 7.92 (d, J=7.6 Hz, 1H), 7.16 (d, J=12.0 Hz, 1H), 4.77-4.67 (m, 1H), 4.55 (s, 2H), 3.57 (t, J=6.0 Hz, 2H), 3.33-3.28 (m, 2H), 2.80 (t, J=6.0 Hz, 2H), 2.57-2.47 (m, 2H), 2.36 (t, J=7.2 Hz, 2H), 2.32-2.22 (m, 2H), 1.91-1.78 (m, 2H), 1.74-1.64 (m, 4H), 1.44 (s, 9H). m/z: [ESI⁺] 610 (M+H)⁺.

### Synthesis of tert-butyl (S)-2-(3-fluoro-4-(7-((3-(4-fluoropiperidin-1-yl)propyl)carbamoyl)-6-methylbenzo[d]imidazo[2,1-b]thiazol-2-yl)phenyl)pyrrolidine-1-carboxylate

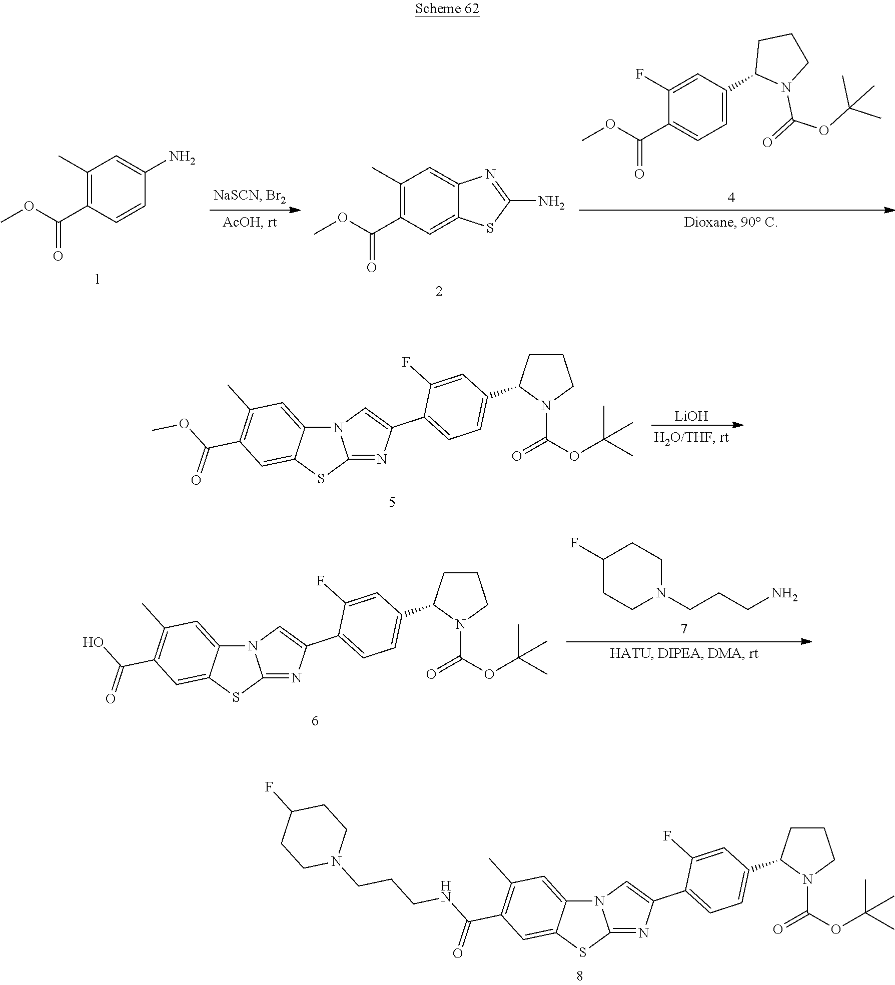

### Synthesis of methyl 2-amino-5-methylbenzo[d]thiazole-6-carboxylate (2, Scheme 62)

To a stirred solution of methyl 4-amino-2-methylbenzoate (5.00 g, 30.27 mmol) and sodium thiocyanate (12.27 g, 151.35 mmol) in acetic acid (70 mL) was added bromine (5.32 g, 33.29 mmol) dropwise at 0° C. under a nitrogen atmosphere. The resulting solution was stirred for 16 h at room temperature. The reaction mixture was then concentrated under vacuum. The residue was purified by silica gel column chromatography (1%-50% of ethyl acetate in petroleum ether) to afford a mixture of two regioisomers. The mixture was separated by Prep-SFC using the following conditions; Column: CHIRALPAK IF, 3×25 cm, 5 μm; Mobile Phase A: carbon dioxide, Mobile Phase B: methanol (plus 0.1% 2 N ammonia-methanol); Flow rate: 100 mL/min; Gradient: isocratic 20% B in 10 min; Detector: UV 220 nm. The slower eluting peak at 6.8 min was collected and concentrated under reduced pressure to afford methyl 2-amino-5-methylbenzo[d]thiazole-6-carboxylate as a white solid.

Yield 1.32 g (20%). ¹H NMR (300 MHz, CDCl₃) δ 8.25 (s, 1H), 7.42 (s, 1H), 5.76 (br s, 2H), 3.92 (s, 3H), 2.70 (s, 3H). m/z: [ESI⁺] 223 (M+H)⁺.

### Synthesis of methyl (S)-2-(4-(1-(tert-butoxycarbonyl)pyrrolidin-2-yl)-2-fluorophenyl)-6-methylbenzo[d]imidazo[2,1-b]thiazole-7-carboxylate (5, Scheme 62)

Compound methyl (S)-2-(4-(1-(tert-butoxycarbonyl)pyrrolidin-2-yl)-2-fluorophenyl)-6-methylbenzo[d]imidazo[2,1-b]thiazole-7-carboxylate was prepared from methyl 2-amino-5-methylbenzo[d]thiazole-6-carboxylate (500 mg, 2.250 mmol) and tert-butyl (S)-2-(4-(2-bromoacetyl)-3-fluorophenyl)pyrrolidine-1-carboxylate (869 mg, 2.250 mmol) following a similar procedure to that described for the synthesis of tert-butyl (S)-2-(4-(7-bromo-9-methyl-9H-benzo[d]imidazo[1,2-a]imidazol-2-yl)-3-fluorophenyl)pyrrolidine-1-carboxylate and was isolated as a light yellow solid.

Yield 280 mg (24%). ¹H NMR (300 MHz, CDCl₃) δ 8.36 (s, 1H), 8.22-8.10 (m, 2H), 7.56 (s, 1H), 7.10 (dd, J=1.5, 8.4 Hz, 1H), 7.00 (dd, J=1.8, 12.6 Hz, 1H), 5.04-4.74 (m, 1H), 3.96 (s, 3H), 3.72-3.60 (m, 2H), 2.79 (s, 3H), 2.46-2.27 (m, 1H), 2.00-1.82 (m, 3H), 1.50-1.25 (m, 9H). m/z: [ESI⁺] 510 (M+H)⁺.

### Synthesis of (S)-2-(4-(1-(tert-butoxycarbonyl)pyrrolidin-2-yl)-2-fluorophenyl)-6-methylbenzo[d]imidazo[2,1-b]thiazole-7-carboxylic Acid (6, Scheme 62)

Compound (S)-2-(4-(1-(tert-butoxycarbonyl)pyrrolidin-2-yl)-2-fluorophenyl)-6-methylbenzo[d]imidazo[2,1-b]thiazole-7-carboxylic acid was prepared from methyl (S)-2-(4-(1-(tert-butoxycarbonyl)pyrrolidin-2-yl)-2-fluorophenyl)-6-methylbenzo[d]imidazo[2,1-b]thiazole-7-carboxylate (280 mg, 0.549 mmol) following a similar procedure to that described for the synthesis of 2-(1-(tert-butoxycarbonyl)-5-fluoroindolin-6-yl)benzo[d]imidazo[2,1-b]thiazole-7-carboxylic acid at room temperature, and was isolated as a light yellow solid.

Yield 250 mg (92%). ¹H NMR (400 MHz, CDCl₃) δ 8.18 (s, 1H), 8.08-8.00 (m, 1H), 7.59 (s, 1H), 7.34-7.21 (m, 1H), 7.11 (d, J=8.0 Hz, 1H), 7.01 (dd, J=4.8, 12.0 Hz, 1H), 5.06-4.76 (m, 1H), 3.74-3.53 (m, 2H), 2.73 (s, 3H), 2.45-2.28 (m, 1H), 1.98-1.83 (m, 3H), 1.57 (s, 3H, part of t-Bu), 1.25 (s, 6H, part of t-Bu). OH proton not observed. m/z: [ESI⁺] 496 (M+H)⁺.

### Synthesis of tert-butyl (S)-2-(3-fluoro-4-(7-((3-(4-fluoropiperidin-1-yl)propyl)carbamoyl)-6-methylbenzo[d]imidazo[2,1-b]thiazol-2-yl)phenyl)pyrrolidine-1-carboxylate (8, Scheme 62)

Compound tert-butyl (S)-2-(3-fluoro-4-(7-((3-(4-fluoropiperidin-1-yl)propyl)carbamoyl)-6-methylbenzo[d]imidazo[2,1-b]thiazol-2-yl)phenyl)pyrrolidine-1-carboxylate was prepared from (S)-2-(4-(1-(tert-butoxycarbonyl)pyrrolidin-2-yl)-2-fluorophenyl)-6-methylbenzo[d]imidazo[2,1-b]thiazole-7-carboxylic acid (250 mg, 0.504 mmol) and 3-(4-fluoropiperidin-1-yl)propan-1-amine (97 mg, 0.605 mmol) following a similar procedure to that described for the synthesis of tert-butyl 5-fluoro-6-(7-((3-(4-fluoropiperidin-1-yl)propyl)carbamoyl)benzo[d]imidazo[2,1-b]thiazol-2-yl)indoline-1-carboxylate, and was isolated as a light yellow solid.

Yield 150 mg (47%). ¹H NMR (400 MHz, CDCl₃) δ 8.20-8.09 (m, 2H), 7.82 (s, 1H), 7.75 (s, 1H), 7.53 (s, 1H), 7.09 (dd, J=1.6, 8.0 Hz, 1H), 6.99 (dd, J=1.6, 12.4 Hz, 1H), 5.04-4.77 (m, 1H), 4.65 (d, J=48.4 Hz, 1H), 3.73-3.49 (m, 4H), 2.69-2.45 (m, 7H), 2.01-1.58 (m, 1H), 1.97-1.60 (m, 11H), 1.50-1.25 (m, 9H). m/z: [ESI⁺] 638 (M+H)⁺.

### Synthesis of tert-butyl (R)-2-(3-fluoro-4-(7-((3-(4-fluoropiperidin-1-yl)propyl)carbamoyl)-6-methylbenzo[d]imidazo[2,1-b]thiazol-2-yl)phenyl)pyrrolidine-1-carboxylate

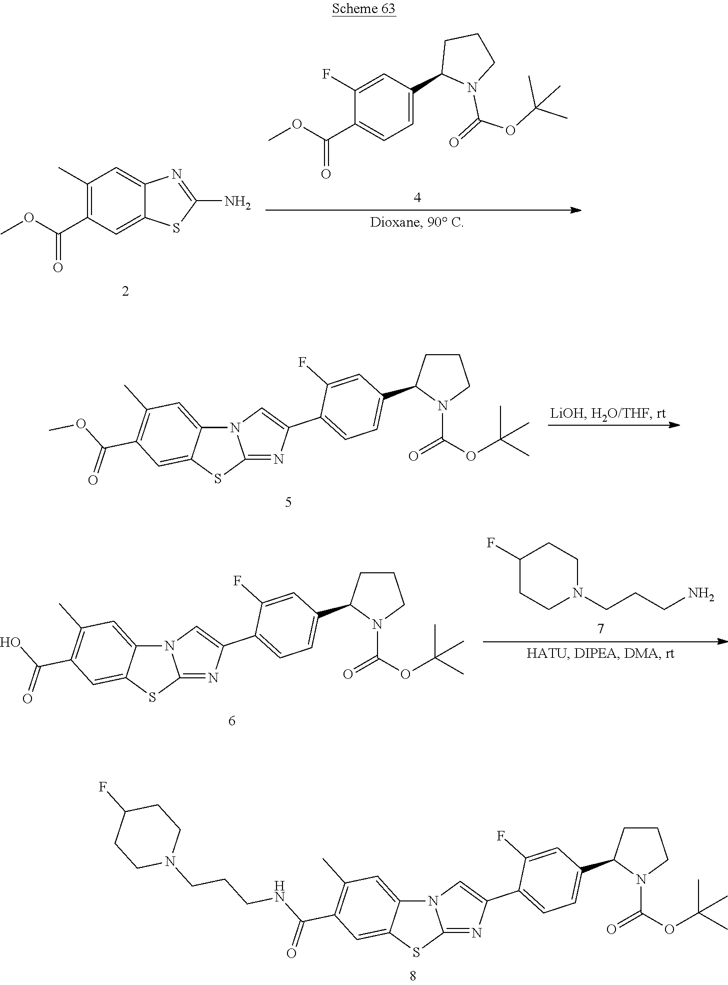

### Synthesis of methyl (R)-2-(4-(1-(tert-butoxycarbonyl)pyrrolidin-2-yl)-2-fluorophenyl)-6-methylbenzo[d]imidazo[2,1-b]thiazole-7-carboxylate (5, Scheme 63)

Compound methyl (R)-2-(4-(1-(tert-butoxycarbonyl)pyrrolidin-2-yl)-2-fluorophenyl)-6-methylbenzo[d]imidazo[2,1-b]thiazole-7-carboxylate was prepared from methyl 2-amino-5-methylbenzo[d]thiazole-6-carboxylate (316 mg, 1.422 mmol) and tert-butyl (R)-2-(4-(2-bromoacetyl)-3-fluorophenyl)pyrrolidine-1-carboxylate (500 mg, 1.294 mmol) following a similar procedure to that described for the synthesis of tert-butyl (S)-2-(4-(7-bromo-9-methyl-9H-benzo[d]imidazo[1,2-a]imidazol-2-yl)-3-fluorophenyl)pyrrolidine-1-carboxylate, and was isolated as a light yellow solid.

Yield 200 mg (30%). ¹H NMR (300 MHz, CDCl₃) δ 8.36 (s, 1H), 8.22-8.10 (m, 2H), 7.56 (s, 1H), 7.10 (dd, J=1.5, 8.4 Hz, 1H), 7.00 (dd, J=1.8, 12.6 Hz, 1H), 5.04-4.74 (m, 1H), 3.96 (s, 3H), 3.72-3.60 (m, 2H), 2.79 (s, 3H), 2.46-2.27 (m, 1H), 2.00-1.82 (m, 3H), 1.50-1.25 (m, 9H). m/z: [ESI⁺] 510 (M+H)⁺.

### Synthesis of (R)-2-(4-(1-(tert-butoxycarbonyl)pyrrolidin-2-yl)-2-fluorophenyl)-6-methylbenzo[d]imidazo[2,1-b]thiazole-7-carboxylic Acid (6, Scheme 63)

Compound (R)-2-(4-(1-(tert-butoxycarbonyl)pyrrolidin-2-yl)-2-fluorophenyl)-6-methylbenzo[d]imidazo[2,1-b]thiazole-7-carboxylic acid was prepared from methyl (R)-2-(4-(1-(tert-butoxycarbonyl)pyrrolidin-2-yl)-2-fluorophenyl)-6-methylbenzo[d]imidazo[2,1-b]thiazole-7-carboxylate (200 mg, 0.392 mmol) following a similar procedure to that described for the synthesis of 2-(1-(tert-butoxycarbonyl)-5-fluoroindolin-6-yl)benzo[d]imidazo[2,1-b]thiazole-7-carboxylic acid at room temperature, and was isolated as a light yellow solid.

Yield 190 mg (98%). ¹H NMR (400 MHz, CDCl₃) δ 8.18 (s, 1H), 8.08-8.00 (m, 1H), 7.59 (s, 1H), 7.34-7.21 (m, 1H), 7.11 (d, J=8.0 Hz, 1H), 7.01 (dd, J=4.8, 12.0 Hz, 1H), 5.06-4.76 (m, 1H), 3.74-3.53 (m, 2H), 2.73 (s, 3H), 2.45-2.28 (m, 1H), 1.98-1.83 (m, 3H), 1.57-1.25 (m, 9H). OH proton not observed. m/z: [ESI⁺] 496 (M+H)⁺.

### Synthesis of tert-butyl (R)-2-(3-fluoro-4-(7-((3-(4-fluoropiperidin-1-yl)propyl)carbamoyl)-6-methylbenzo[d]imidazo[2,1-b]thiazol-2-yl)phenyl)pyrrolidine-1-carboxylate (8, Scheme 63)

Compound tert-butyl (R)-2-(3-fluoro-4-(7-((3-(4-fluoropiperidin-1-yl)propyl)carbamoyl)-6-methylbenzo[d]imidazo[2,1-b]thiazol-2-yl)phenyl)pyrrolidine-1-carboxylate was prepared from (R)-2-(4-(1-(tert-butoxycarbonyl)pyrrolidin-2-yl)-2-fluorophenyl)-6-methylbenzo[d]imidazo[2,1-b]thiazole-7-carboxylic acid (190 mg, 0.383 mmol) and 3-(4-fluoropiperidin-1-yl)propan-1-amine (78 mg, 0.487 mmol) following a similar procedure to that described for the synthesis of tert-butyl 5-fluoro-6-(7-((3-(4-fluoropiperidin-1-yl)propyl)carbamoyl)benzo[d]imidazo[2,1-b]thiazol-2-yl)indoline-1-carboxylate, and was isolated as a light yellow solid.

Yield 110 mg (45%). ¹H NMR (400 MHz, CDCl₃) δ 8.20-8.09 (m, 2H), 7.82 (s, 1H), 7.75 (s, 1H), 7.53 (s, 1H), 7.09 (dd, J=1.6, 8.0 Hz, 1H), 6.99 (dd, J=1.6, 12.4 Hz, 1H), 5.04-4.77 (m, 1H), 4.65 (d, J=48.4 Hz, 1H), 3.73-3.49 (m, 4H), 2.69-2.45 (m, 7H), 2.01-1.58 (m, 1H), 1.97-1.60 (m, 11H), 1.50-1.25 (m, 9H). m/z: [ESI⁺] 638 (M+H)⁺.

### Synthesis of tert-butyl (S)-2-(3-fluoro-4-(7-((3-(4-fluoropiperidin-1-yl)propyl)carbamoyl)-9H-benzo[d]imidazo[1,2-a]imidazol-2-yl)phenyl)pyrrolidine-1-carboxylate

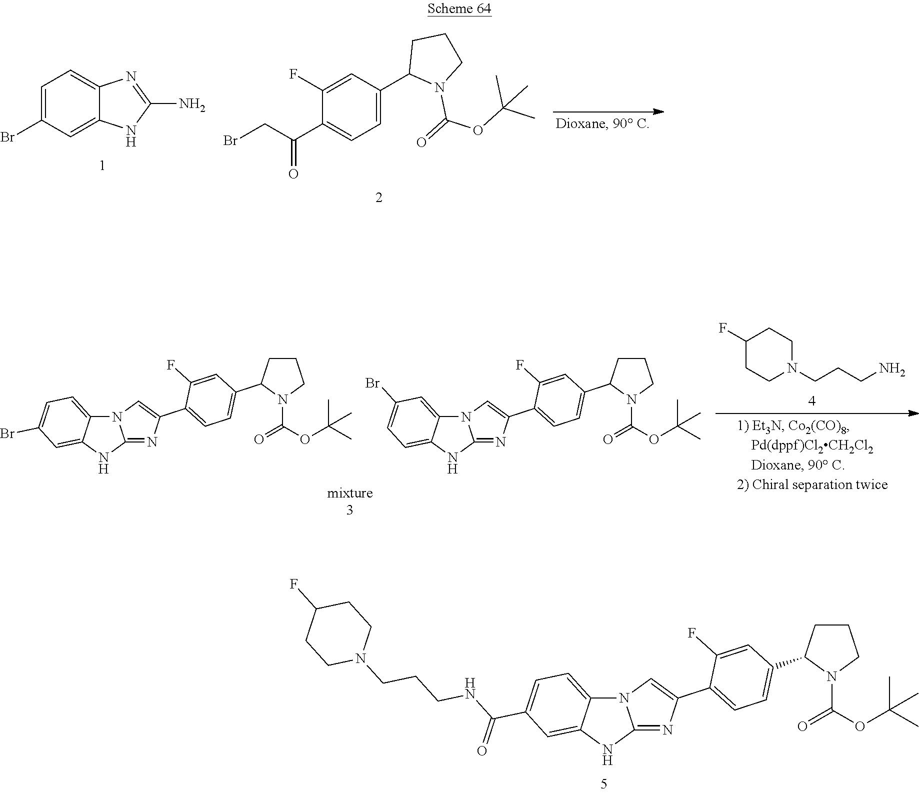

### Synthesis of mixture of tert-butyl 2-(4-(7-bromo-9H-benzo[d]imidazo[1,2-a]imidazol-2-yl)-3-fluorophenyl)pyrrolidine-1-carboxylate and tert-butyl 2-(4-(6-bromo-9H-benzo[d]imidazo[,2-a]imidazol-2-yl)-3-fluorophenyl)pyrrolidine-1-carboxylate (3, Scheme 64)

A mixture of tert-butyl 2-(4-(7-bromo-9H-benzo[d]imidazo[1,2-a]imidazol-2-yl)-3-fluorophenyl)pyrrolidine-1-carboxylate and tert-butyl 2-(4-(6-bromo-9H-benzo[d]imidazo[1,2-a]imidazol-2-yl)-3-fluorophenyl)pyrrolidine-1-carboxylate was prepared from 6-bromo-1H-benzo[d]imidazol-2-amine (3.84 g, 18.11 mmol) and tert-butyl 2-(4-(2-bromoacetyl)-3-fluorophenyl)pyrrolidine-1-carboxylate (3.50 g, 9.06 mmol) following a similar procedure to that described for the synthesis of tert-butyl (S)-2-(4-(7-bromo-9-methyl-9H-benzo[d]imidazo[1,2-a]imidazol-2-yl)-3-fluorophenyl)pyrrolidine-1-carboxylate, and was isolated as a yellow solid.

Yield 3.00 g (66%). ¹H NMR (300 MHz, CDCl₃) δ 8.10-7.92 (m, 1H), 7.84-7.68 (m, 1H), 7.62-7.56 (m, 1H), 7.50-7.41 (m, 1H), 7.41-7.30 (m, 1H), 7.18-7.10 (m, 1H), 7.10-6.99 (m, 1H), 5.08-4.81 (m, 1H), 3.77-3.53 (m, 2H), 2.47-2.29 (m, 1H), 2.06-1.83 (m, 3H), 1.51-1.25 (m, 9H). NH proton not observed. m/z: [ESI⁺] 499, 501 (M+H)⁺.

### Synthesis of tert-butyl (S)-2-(3-fluoro-4-(7-((3-(4-fluoropiperidin-1-yl)propyl)carbamoyl)-9H-benzo[d]imidazo[1,2-a]imidazol-2-yl)phenyl)pyrrolidine-1-carboxylate (5, Scheme 64)

A mixture of tert-butyl 2-(3-fluoro-4-(6-((3-(4-fluoropiperidin-1-yl)propyl)carbamoyl)-9H-benzo[d]imidazo[1,2-a]imidazol-2-yl)phenyl)pyrrolidine-1-carboxylate/tert-butyl 2-(3-fluoro-4-(7-((3-(4-fluoropiperidin-1-yl)propyl)carbamoyl)-9H-benzo[d]imidazo[1,2-a]imidazol-2-yl)phenyl)pyrrolidine-1-carboxylate was prepared from a mixture of tert-butyl 2-(4-(7-bromo-9H-benzo[d]imidazo[1,2-a]imidazol-2-yl)-3-fluorophenyl)pyrrolidine-1-carboxylate/tert-butyl 2-(4-(6-bromo-9H-benzo[d]imidazo[1,2-a]imidazol-2-yl)-3-fluorophenyl)pyrrolidine-1-carboxylate (650 mg, 1.302 mmol) and 3-(4-fluoropiperidin-1-yl)propan-1-amine (420 mg, 2.621 mmol) following a similar procedure to that described for the synthesis of tert-butyl (S)-2-(3-fluoro-4-(7-((3-(4-fluoropiperidin-1-yl)propyl)carbamoyl)-9-methyl-9H-benzo[d]imidazo[1,2-a]imidazol-2-yl)phenyl)pyrrolidine-1-carboxylate, and was isolated as a white solid (500 mg) m/z: [ESI⁺] 607 (M+H)⁺.

The two regioisomers (500 mg) were chiral separated by Prep-CHIRAL-HPLC with the following conditions; Column: CHIRALPAK IG, 2×25 cm, 5 μm; Mobile Phase A: Hexane (plus 0.5% 2 N ammonium-methanol, Mobile Phase B: ethanol:dichloromethane=1:1; Flow rate: 20 mL/min; Gradient: isocratic 40% B in 23 min; Detector: UV 254/220 nm. The third eluting peak at 18.72 min was collected and concentrated under reduced pressure to afford 85 mg of a mixture, which was subjected to chiral separation by Prep-CHIRAL-HPLC with the following conditions; Column: CHIRAL ART Cellulose-SC, 2×25 cm, 5 μm; Mobile Phase A: hexane (plus 0.5% 2 N ammonium-methanol), Mobile Phase B: ethanol:dichloromethane=1:1; Flow rate: 20 mL/min; Gradient: isocratic 40% B in 11 min; Detector: UV 254/220 nm. The slower eluting peak at 9.09 min was collected and concentrated under reduced pressure to afford tert-butyl (S)-2-(3-fluoro-4-(7-((3-(4-fluoropiperidin-1-yl)propyl)carbamoyl)-9H-benzo[d]imidazo[1,2-a]imidazol-2-yl)phenyl)pyrrolidine-1-carboxylate as a white solid.

Yield 40 mg (5%). H NMR (400 MHz, DMSO) δ 11.96 (br s, 1H), 8.54 (t, J=5.6 Hz, 1H), 8.11 (d, J=3.6 Hz, 1H), 8.05 (d, J=8.0 Hz, 1H), 7.92 (d, J=8.4 Hz, 1H), 7.88 (s, 1H), 7.70 (dd, J=1.6, 8.4 Hz, 1H), 7.15-7.01 (m, 2H), 4.87-4.54 (m, 2H), 3.62-3.41 (m, 3H), 2.60-2.50 (m, 4H), 2.42-2.22 (m, 5H), 1.93-1.78 (m, 4H), 1.78-1.62 (m, 4H), 1.41-1.14 (m, 9H). m/z: [ESI⁺] 607 (M+H)⁺. [a]²⁵_{D}=−50° (c=1 mg/mL, methanol).

### Synthesis of tert-butyl (S)-2-(3-fluoro-4-(7-((3-(4-fluoropiperidin-1-yl)propyl)carbamoyl)-3-methylbenzo[d]imidazo[2,1-b]thiazol-2-yl)phenyl)pyrrolidine-1-carboxylate and tert-butyl (R)-2-(3-fluoro-4-(7-((3-(4-fluoropiperidin-1-yl)propyl)carbamoyl)-3-methylbenzo[d]imidazo[2,1-b]thiazol-2-yl)phenyl)pyrrolidine-1-carboxylate

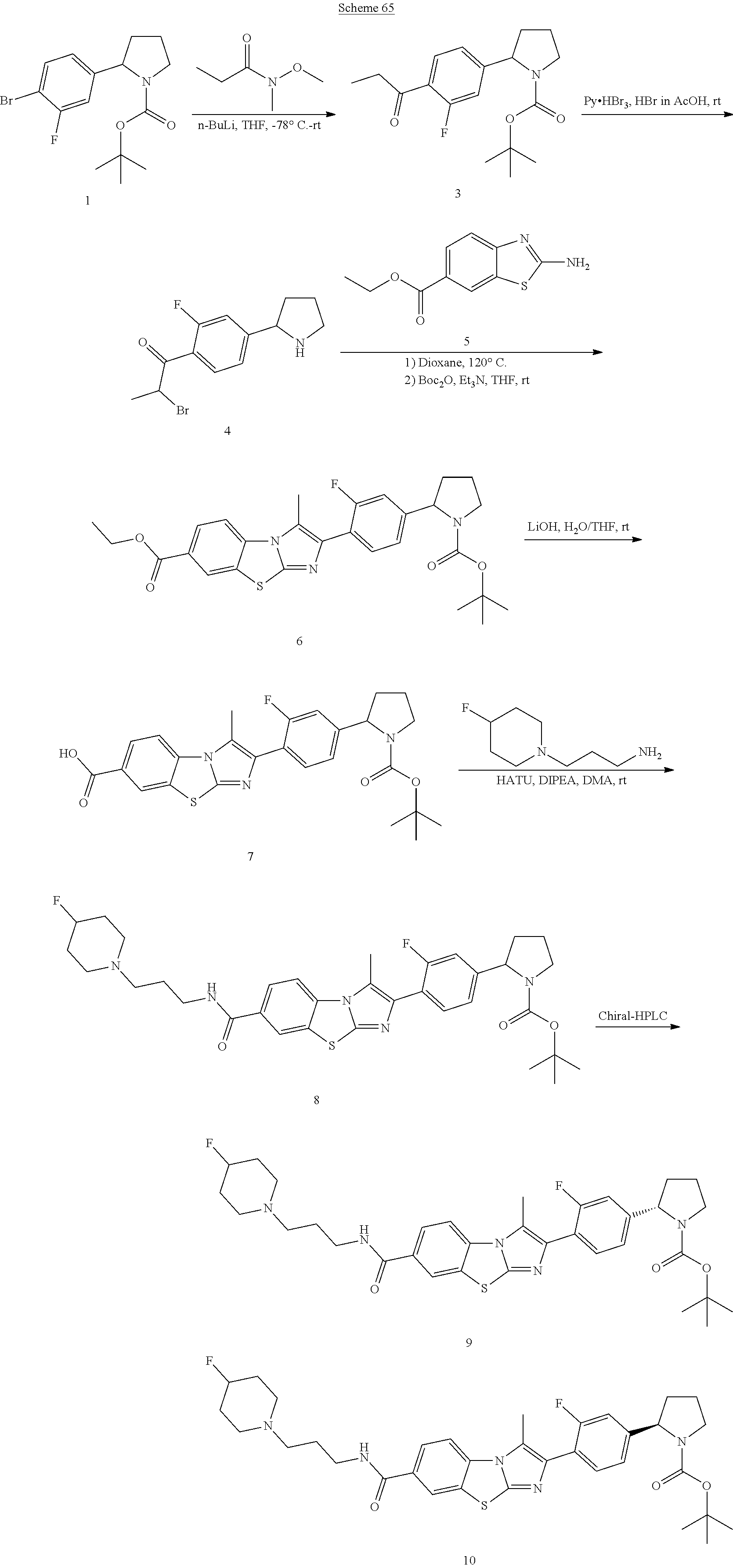

### Synthesis of tert-butyl 2-(3-fluoro-4-propionylphenyl)pyrrolidine-1-carboxylate (3, Scheme 65)

A solution of tert-butyl 2-(4-bromo-3-fluorophenyl)pyrrolidine-1-carboxylate (4.00 g, 11.62 mmol) in tetrahydrofuran (200 mL) was treated with n-butyllithium (2.5 N in hexane, 14 mL, 35.00 mmol) for 30 min at −78° C. under a nitrogen atmosphere. N-methoxy-N-methylpropionamide (2.72 g, 23.22 mmol) was then added dropwise over 5 min. The resulting solution was stirred for 1 h. The solution was allowed to warm up to 0° C. and quenched with water (100 mL). The resulting mixture was concentrated under reduced pressure to remove the organic solvent. The aqueous mixture was then extracted with ethyl acetate (2×200 mL). The combined organic layers were washed with brine (200 mL) and dried over anhydrous sodium sulfate. After filtration, the filtrate was concentrated under reduced pressure. The residue was purified by silica gel column chromatography (0%-50% of ethyl acetate in petroleum ether) to afford tert-butyl 2-(3-fluoro-4-propionylphenyl)pyrrolidine-1-carboxylate as a colorless oil.

Yield 1.20 g (32%). ¹H NMR (400 MHz, DMSO) δ 7.85-7.72 (m, 1H), 7.20-7.07 (m, 2H), 4.90-4.70 (m, 1H), 3.61-3.39 (m, 2H), 2.97 (q, J=7.2 Hz, 2H), 2.39-2.23 (m, 1H), 1.90-1.63 (m, 3H), 1.40-1.12 (m, 9H), 1.08 (t, J=7.2 Hz, 3H). m/z: [ESI⁺] 266 (M+H-56)⁺.

### Synthesis of 2-bromo-1-(2-fluoro-4-(pyrrolidin-2-yl)phenyl)propan-1-one hydrobromide (4, Scheme 65)

A solution of tert-butyl 2-(3-fluoro-4-propionylphenyl)pyrrolidine-1-carboxylate (1.20 g, 3.73 mmol) and pyridinium tribromide (1.20 g, 3.75 mmol) in 40% hydrobromide in acetic acid (12 mL) was stirred for 3 h at room temperature under a nitrogen atmosphere. The reaction mixture was concentrated under reduced pressure to give 2-bromo-1-(2-fluoro-4-(pyrrolidin-2-yl)phenyl)propan-1-one hydrobromide as a brown oil.

Yield 1.30 g (crude). ¹H NMR not run. m/z: [ESI⁺] 300, 302 (M+H)⁺.

### Synthesis of ethyl 2-(4-(1-(tert-butoxycarbonyl)pyrrolidin-2-yl)-2-fluorophenyl)-3-methylbenzo[d]imidazo[2,1-b]thiazole-7-carboxylate (6, Scheme 65)

Compound ethyl 2-(4-(1-(tert-butoxycarbonyl)pyrrolidin-2-yl)-2-fluorophenyl)-3-methylbenzo[d]imidazo[2,1-b]thiazole-7-carboxylate was prepared from 2-bromo-1-(2-fluoro-4-(pyrrolidin-2-yl)phenyl)propan-1-one hydrobromide (1.30 g, crude) and ethyl 2-aminobenzo[d]thiazole-6-carboxylate (1.52 g, 6.84 mmol) following a similar procedure to that described for the synthesis of tert-butyl (S)-2-(4-(7-bromo-9-methyl-9H-benzo[d]imidazo[1,2-a]imidazol-2-yl)-3-fluorophenyl)pyrrolidine-1-carboxylate at 120° C. Upon completion, Boc protection was carried out following a similar procedure to that described for the synthesis of tert-butyl 6-bromo-5-fluoroindoline-1-carboxylate, and was isolated as a light brown solid.

Yield 0.92 g (47% over two steps). ¹H NMR (400 MHz, DMSO) δ 8.70 (d, J=1.6 Hz, 1H), 8.16-8.06 (m, 2H), 7.60-7.51 (m, 1H), 7.20-7.07 (m, 2H), 4.97-4.70 (m, 1H), 4.38 (q, J=7.2 Hz, 2H), 3.64-3.43 (m, 2H), 2.69 (s, 3H), 2.42-2.23 (m, 1H), 1.93-1.73 (m, 3H), 1.42-1.15 (m, 12H). m/z: [ESI⁺] 524 (M+H)⁺.

### Synthesis of 2-(4-(1-(tert-butoxycarbonyl)pyrrolidin-2-yl)-2-fluorophenyl)-3-methylbenzo[d]imidazo[2,1-b]thiazole-7-carboxylic Acid (7, Scheme 65)

Compound 2-(4-(1-(tert-butoxycarbonyl)pyrrolidin-2-yl)-2-fluorophenyl)-3-methylbenzo[d]imidazo[2,1-b]thiazole-7-carboxylic acid was prepared from ethyl 2-(4-(1-(tert-butoxycarbonyl)pyrrolidin-2-yl)-2-fluorophenyl)-3-methylbenzo[d]imidazo[2,1-b]thiazole-7-carboxylate (0.92 g, 1.76 mmol) following a similar procedure to that described for the synthesis of 2-(1-(tert-butoxycarbonyl)-5-fluoroindolin-6-yl)benzo[d]imidazo[2,1-b]thiazole-7-carboxylic acid at room temperature, and was isolated as an off-white solid.

Yield 0.57 g (65%). ¹H NMR (400 MHz, DMSO) δ 13.21 (br s, 1H), 8.63 (s, 1H), 8.16-8.06 (m, 2H), 7.62-7.51 (m, 1H), 7.19-7.06 (m, 2H), 4.95-4.69 (m, 1H), 3.66-3.40 (m, 2H), 2.69 (s, 3H), 2.42-2.23 (m, 1H), 1.92-1.72 (m, 3H), 1.42-1.15 (m, 9H). m/z: [ESI⁺] 496 (M+H)⁺.

### Synthesis of tert-butyl 2-(3-fluoro-4-(7-((3-(4-fluoropiperidin-1-yl)propyl)carbamoyl)-3-methylbenzo[d]imidazo[2,1-b]thiazol-2-yl)phenyl)pyrrolidine-1-carboxylate (8, Scheme 65)

Compound tert-butyl 2-(3-fluoro-4-(7-((3-(4-fluoropiperidin-1-yl)propyl)carbamoyl)-3-methylbenzo[d]imidazo[2,1-b]thiazol-2-yl)phenyl)pyrrolidine-1-carboxylate was prepared from 2-(4-(1-(tert-butoxycarbonyl)pyrrolidin-2-yl)-2-fluorophenyl)-3-methylbenzo[d]imidazo[2,1-b]thiazole-7-carboxylic acid (570 mg, 1.150 mmol) and 3-(4-fluoropiperidin-1-yl)propan-1-amine (368 mg, 2.297) following a similar procedure to that described for the synthesis of tert-butyl 5-fluoro-6-(7-((3-(4-fluoropiperidin-1-yl)propyl)carbamoyl)benzo[d]imidazo[2,1-b]thiazol-2-yl)indoline-1-carboxylate, and was isolated as a brown oil.

Yield 210 mg (29%). ¹H NMR (300 MHz, CD₃OD) δ 8.34 (d, J=1.5 Hz, 1H), 8.07 (d, J=8.7 Hz, 1H), 7.99 (dd, J=1.5, 8.7 Hz, 1H), 7.61-7.50 (m, 1H), 7.15 (dd, J=1.5, 8.1 Hz, 1H), 7.08 (d, J=11.4 Hz, 1H), 4.79-4.54 (m, 1H), 3.72-3.56 (m, 3H), 3.48 (t, J=6.9 Hz, 2H), 2.75-2.62 (m, 5H), 2.60-2.36 (m, 4H), 2.05 (s, 3H), 2.01-1.79 (m, 4H), 1.69-1.56 (m, 1H), 1.55-1.42 (m, 2H), 1.30 (s, 9H). NH proton not observed. m/z: [ESI⁺] 638 (M+H)⁺.

### Synthesis of tert-butyl (S)-2-(3-fluoro-4-(7-((3-(4-fluoropiperidin-1-yl)propyl)carbamoyl)-3-methylbenzo[d]imidazo[2,1-b]thiazol-2-yl)phenyl)pyrrolidine-1-carboxylate and tert-butyl (R)-2-(3-fluoro-4-(7-((3-(4-fluoropiperidin-1-yl)propyl)carbamoyl)-3-methylbenzo[d]imidazo[2,1-b]thiazol-2-yl)phenyl)pyrrolidine-1-carboxylate (9, 10, Scheme 65)

Tert-butyl 2-(3-fluoro-4-(7-((3-(4-fluoropiperidin-1-yl)propyl)carbamoyl)-3-methylbenzo[d]imidazo[2,1-b]thiazol-2-yl)phenyl)pyrrolidine-1-carboxylate (240 mg, 0.376 mmol) was separated by Prep-Chiral-HPLC with the following conditions; Column: CHIRALPAK IG, 2×25 cm, 5 μm; Mobile Phase A: hexane (plus 0.5% 2 N ammonium-methanol), Mobile Phase B: ethanol:dichloromethane=1:1; Flow rate: 20 mL/min; Gradient: isocratic 30% B in 18 min; Detector: UV 254/220 nm. The faster eluting peak at 13.02 min was collected and concentrated under reduced pressure to afford tert-butyl (S)-2-(3-fluoro-4-(7-((3-(4-fluoropiperidin-1-yl)propyl)carbamoyl)-3-methylbenzo[d]imidazo[2,1-b]thiazol-2-yl)phenyl)pyrrolidine-1-carboxylate as a colorless oil.

Yield 46 mg (19%). ¹H NMR (300 MHz, CD₃OD) δ 8.34 (d, J=1.5 Hz, 1H), 8.07 (d, J=8.7 Hz, 1H), 7.99 (dd, J=1.5, 8.7 Hz, 1H), 7.61-7.50 (m, 1H), 7.15 (dd, J=1.5, 8.1 Hz, 1H), 7.08 (d, J=11.4 Hz, 1H), 4.79-4.54 (m, 1H), 3.72-3.56 (m, 3H), 3.48 (t, J=6.9 Hz, 2H), 2.75-2.62 (m, 5H), 2.60-2.36 (m, 4H), 2.05 (s, 3H), 2.01-1.79 (m, 4H), 1.69-1.56 (m, 1H), 1.55-1.42 (m, 2H), 1.30 (s, 9H). NH proton not observed. m/z: [ESI⁺] 638 (M+H)⁺.

The slower eluting peak at 15.78 min was collected and concentrated under reduced pressure to afford tert-butyl (R)-2-(3-fluoro-4-(7-((3-(4-fluoropiperidin-1-yl)propyl)carbamoyl)-3-methylbenzo[d]imidazo[2,1-b]thiazol-2-yl)phenyl)pyrrolidine-1-carboxylate as a colorless oil.

Yield 38 mg (16%). ¹H NMR (300 MHz, CD₃OD) δ 8.34 (d, J=1.5 Hz, 1H), 8.07 (d, J=8.7 Hz, 1H), 7.99 (dd, J=1.5, 8.7 Hz, 1H), 7.61-7.50 (m, 1H), 7.15 (dd, J=1.5, 8.1 Hz, 1H), 7.08 (d, J=11.4 Hz, 1H), 4.79-4.54 (m, 1H), 3.72-3.56 (m, 3H), 3.48 (t, J=6.9 Hz, 2H), 2.75-2.62 (m, 5H), 2.60-2.36 (m, 4H), 2.05 (s, 3H), 2.01-1.79 (m, 4H), 1.69-1.56 (m, 1H), 1.55-1.42 (m, 2H), 1.30 (s, 9H). NH proton not observed. m/z: [ESI⁺] 638 (M+H)⁺.

### Synthesis of tert-butyl 2-(4-(7-((3-(4-fluoropiperidin-1-yl)propyl)carbamoyl)-6-(2-methoxyethoxy)benzo[d]imidazo[2,1-b]thiazol-2-yl)phenyl)pyrrolidine-1-carboxylate

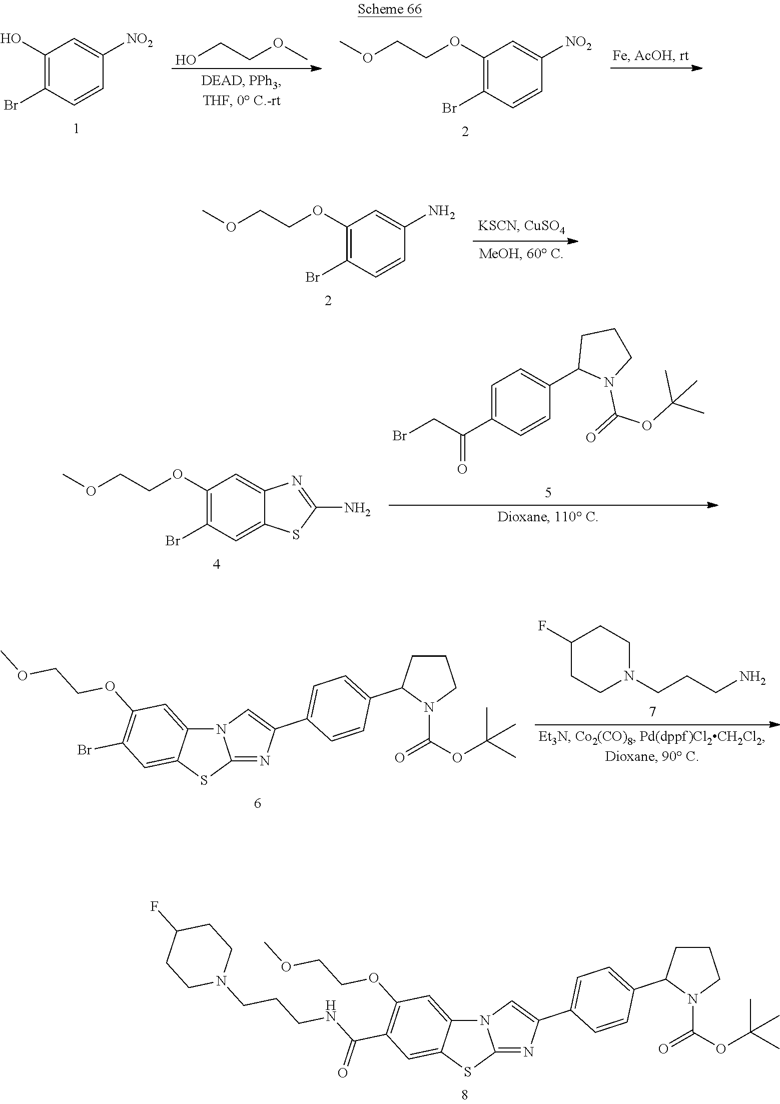

### Synthesis of 1-bromo-2-(2-methoxyethoxy)-4-nitrobenzene (2, Scheme 66)

Diethyl azodicarboxylate (5.99 g, 34.39 mmol) was added dropwise over 1 min into a solution of 2-bromo-5-nitrophenol (5.00 g, 22.93 mmol), triphenylphosphine (9.02 g, 34.39 mmol) and 2-methoxyethan-1-ol (3.49 g, 45.86 mmol) in tetrahydrofuran (50 mL) at 0° C. The reaction solution was stirred for 2 h at room temperature. The reaction mixture was diluted with water (100 mL) and extracted with ethyl acetate (3×100 mL). The combined organic layers were washed with brine (200 mL) and dried over anhydrous sodium sulfate. After filtration, the filtrate was concentrated under reduced pressure. The residue was purified by silica gel column chromatography (25% of ethyl acetate in petroleum ether) to afford 1-bromo-2-(2-methoxyethoxy)-4-nitrobenzene as a light yellow solid.

Yield 5.10 g (81%). ¹H NMR (400 MHz, CDCl₃) δ 7.80-7.79 (m, 1H), 7.75-7.73 (m, 2H), 4.32-4.28 (m, 2H), 3.89-3.85 (m, 2H), 3.51 (s, 3H). No mass ion observed.

### Synthesis of 4-bromo-3-(2-methoxyethoxy)aniline (3, Scheme 66)

A mixture of 1-bromo-2-(2-methoxyethoxy)-4-nitrobenzene (3.00 g, 10.87 mmol) and iron powder (3.03 g, 54.25 mmol) in acetic acid (30 mL) was stirred for 2 h at room temperature. The resulting mixture was filtered. The filtered cake was washed with ethyl acetate (3×50 mL). The combined filtrate was concentrated under reduced pressure. The residue was diluted with water (50 mL) and adjusted the pH to 7 with an aqueous solution of saturated sodium bicarbonate. The resulting mixture was extracted with ethyl acetate (3×100 mL). The combined organic phase was washed with brine (200 mL) and dried over anhydrous sodium sulfate. After filtration, the filtrate was concentrated under reduced pressure to afford 4-bromo-3-(2-methoxyethoxy)aniline as an orange oil.

Yield (2.20 g, 82%). ¹H NMR (300 MHz, CDCl₃) δ 7.25 (d, d, J=8.4 Hz, 1H), 6.30 (d, J=2.4 Hz, 1H), 6.22 (dd, J=2.4, 8.4 Hz, 1H), 4.13 (t, J=5.7 Hz, 2H), 3.81 (t, J=5.7 Hz, 2H), 3.50 (s, 3H). m/z: [ESI⁺]246, 248 (M+H)⁺.

### Synthesis of 6-bromo-5-(2-methoxyethoxy)benzo[d]thiazol-2-amine (4, Scheme 66)

A mixture of 4-bromo-3-(2-methoxyethoxy)aniline (3.50 g, 14.22 mmol), potassium thiocyanate (14.10 g, 145.09 mmol) and copper(II) sulfate (11.35 g, 71.11 mmol) in methanol (40 mL) was stirred for 9 h at 60° C. under a nitrogen atmosphere. The mixture was cooled to room temperature and filtered. The filtered cake was washed with methanol (3×100 mL). The combined filtrate was concentrated under reduced pressure. The residue was purified by silica gel column chromatography (2.5% of methanol in dichloromethane) to afford 6-bromo-5-(2-methoxyethoxy)benzo[d]thiazol-2-amine as a light yellow solid.

Yield 3.80 g (88%). ¹H NMR (400 MHz, DMSO) δ 8.68 (br s, 2H), 8.00 (s, 1H), 7.12 (s, 1H), 4.23-4.15 (m, 2H), 3.76-3.68 (m, 3H), 3.36 (s, 3H). m/z: [ESI⁺] 303, 305 (M+H)⁺.

### Synthesis of tert-butyl 2-(4-(7-bromo-6-(2-methoxyethoxy)benzo[d]imidazo[2,1-b]thiazol-2-yl)phenyl)pyrrolidine-1-carboxylate (6, Scheme 66)

Compound tert-butyl 2-(4-(7-bromo-6-(2-methoxyethoxy)benzo[d]imidazo[2,1-b]thiazol-2-yl)phenyl)pyrrolidine-1-carboxylate was prepared from 6-bromo-5-(2-methoxyethoxy)benzo[d]thiazol-2-amine (0.50 g, 1.65 mmol) and tert-butyl 2-(4-(2-bromoacetyl)phenyl)pyrrolidine-1-carboxylate (0.67 g, 1.82 mmol) following a similar procedure to that described for the synthesis of tert-butyl (S)-2-(4-(7-bromo-9-methyl-9H-benzo[d]imidazo[1,2-a]imidazol-2-yl)-3-fluorophenyl)pyrrolidine-1-carboxylate at 110° C., and was isolated as a yellow solid.

Yield 0.16 g (17%). ¹H NMR (400 MHz, CDCl₃) δ 7.94 (s, 1H), 7.87 (s, 1H), 7.82 (d, J=8.0 Hz, 2H), 7.25 (d, J=8.0 Hz, 2H), 7.23 (s, 1H), 5.06-4.73 (m, 1H), 4.32 (t, J=4.8 Hz, 2H), 3.90 (t, J=4.8 Hz, 2H), 3.76-3.59 (m, 2H), 3.54 (s, 3H), 2.44-2.26 (m, 1H), 2.02-1.81 (m, 3H), 1.47-1.22 (m, 9H). m/z: [ESI⁺] 572, 574 (M+H)⁺.

### Synthesis of tert-butyl 2-(4-(7-((3-(4-fluoropiperidin-1-yl)propyl)carbamoyl)-6-(2-methoxyethoxy)benzo[d]imidazo[2,1-b]thiazol-2-yl)phenyl)pyrrolidine-1-carboxylate (8, Scheme 66)

Compound tert-butyl 2-(4-(7-((3-(4-fluoropiperidin-1-yl)propyl)carbamoyl)-6-(2-methoxyethoxy)benzo[d]imidazo[2,1-b]thiazol-2-yl)phenyl)pyrrolidine-1-carboxylate was prepared from tert-butyl 2-(4-(7-bromo-6-(2-methoxyethoxy)benzo[d]imidazo[2,1-b]thiazol-2-yl)phenyl)pyrrolidine-1-carboxylate (160 mg, 0.279 mmol) and 3-(4-fluoropiperidin-1-yl)propan-1-amine (90 mg, 0.562 mmol) following a similar procedure to that described for the synthesis of tert-butyl (S)-2-(3-fluoro-4-(7-((3-(4-fluoropiperidin-1-yl)propyl)carbamoyl)-9-methyl-9H-benzo[d]imidazo[1,2-a]imidazol-2-yl)phenyl)pyrrolidine-1-carboxylate, and was isolated as a light yellow solid.

Yield 33 mg (17%). ¹H NMR (400 MHz, CDCl₃) δ 8.55 (s, 1H), 8.40 (br s, 1H), 7.95 (s, 1H), 7.83 (d, J=8.0 Hz, 2H), 7.27-7.24 (m, 1H), 7.23 (d, J=8.0 Hz, 2H), 5.20-4.74 (m, 2H), 4.48-4.35 (m, 2H), 4.02-3.84 (m, 2H), 3.73-3.63 (m, 2H), 3.63-3.56 (m, 2H), 3.55 (s, 3H), 3.04-2.63 (m, 2H), 2.41-2.29 (m, 1H), 2.11-1.84 (m, 3H), 1.69-1.53 (m, 10H), 1.49-1.23 (m, 9H). m/z: [ESI⁺] 680 (M+H)⁺.

### Synthesis of tert-butyl 2-(4-(6-(difluoromethoxy)-7-((3-(4-fluoropiperidin-1-yl)propyl)carbamoyl)benzo[d]imidazo[2,1-b]thiazol-2-yl)phenyl)pyrrolidine-1-carboxylate

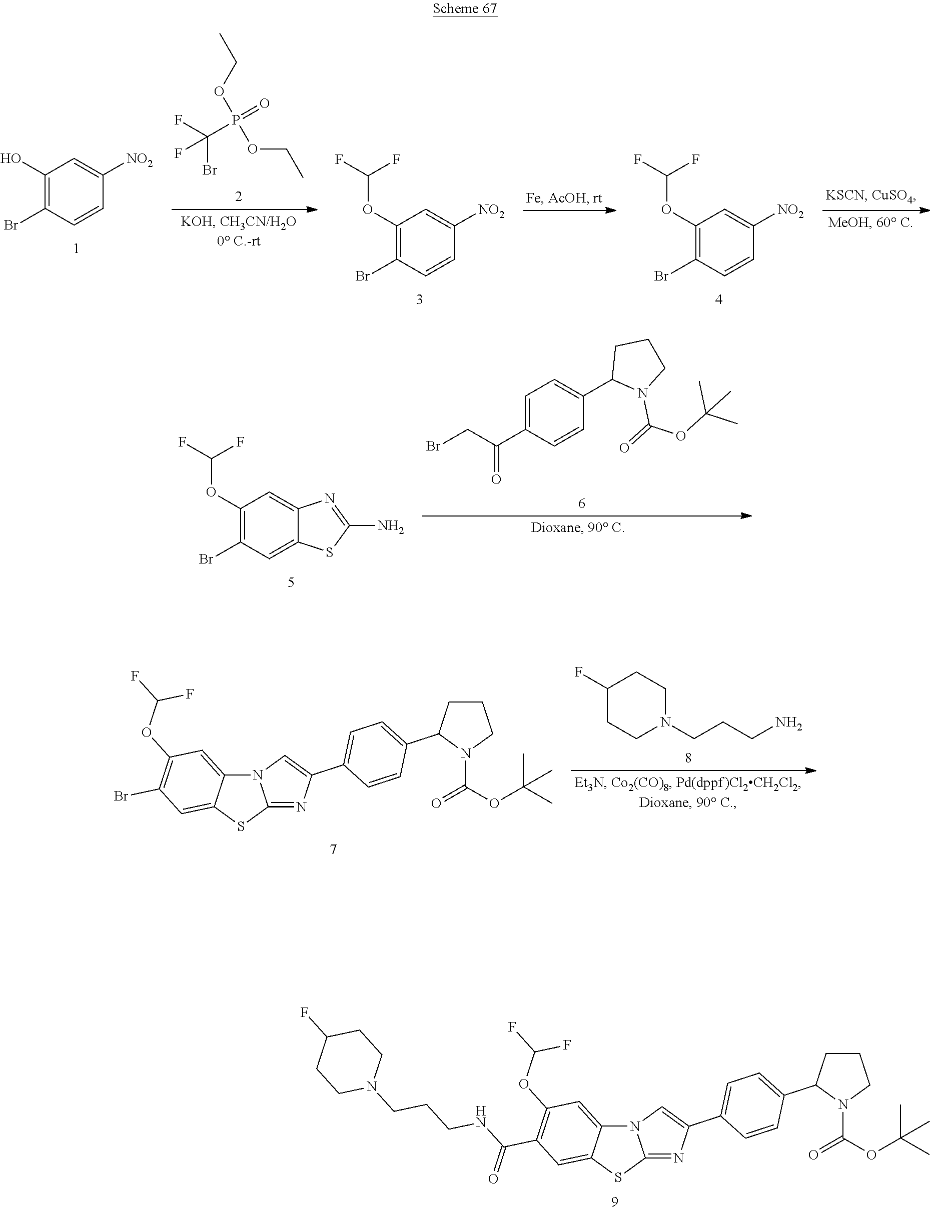

### Synthesis of 1-bromo-2-(difluoromethoxy)-4-nitrobenzene (3, Scheme 67)

To a stirred solution of 2-bromo-5-nitrophenol (5.00 g, 22.93 mmol) and potassium hydroxide (13.00 g, 231.69 mmol) in acetonitrile:water (1:1, 60 mL) was added diethyl bromodifluoromethylphosphonate (12.00 g, 44.94 mmol) dropwise at 0° C. under a nitrogen atmosphere. The reaction solution was allowed to warm to room temperature and stirred for 2 h. The reaction mixture was diluted with water (60 mL) and extracted with ethyl acetate (3×130 mL). The combined organic layers were dried over anhydrous sodium sulfate. After filtration, the filtrate was concentrated under reduced pressure. This resulted in 1-bromo-2-(difluoromethoxy)-4-nitrobenzene as a brown oil.

Yield 6.02 g (98%). ¹H NMR (400 MHz, CDCl₃) δ 8.13 (q, J=2.4 Hz, 1H), 8.02 (dd, J=2.4, 8.8 Hz, 1H), 7.86 (d, J=8.8 Hz, 1H), 6.68 (t, J=72.0 Hz, 1H). No mass ion observed

### Synthesis of 4-bromo-3-(difluoromethoxy)aniline (4, Scheme 67)

Compound 4-bromo-3-(difluoromethoxy)aniline was prepared from 1-bromo-2-(difluoromethoxy)-4-nitrobenzene (6.00 g, 22.39 mmol) following a similar procedure to that described for the synthesis of 4-bromo-3-(2-methoxyethoxy)aniline, and was isolated as a brown solid.

Yield 4.50 g (crude). ¹H NMR not run. m/z: [ESI⁺] 238, 240 (M+H)⁺.

### Synthesis of 6-bromo-5-(difluoromethoxy)benzo[d]thiazol-2-amine (5, Scheme 67)

Compound 6-bromo-5-(difluoromethoxy)benzo[d]thiazol-2-amine was prepared from 4-bromo-3-(difluoromethoxy)aniline (4.50 g, crude) and potassium thiocyanate (11.00 g, 113.19 mmol) following a similar procedure to that described for the synthesis of 6-bromo-5-(2-methoxyethoxy)benzo[d]thiazol-2-amine, and was isolated as an off-white solid.

Yield 2.00 g (30% over two steps). ¹H NMR (400 MHz, CDCl₃) δ 7.81 (s, 1H), 7.42 (s, 1H), 6.55 (t, J=73.6 Hz, 1H), 5.44 (br s, 2H). m/z: [ESI⁺] 295, 297 (M+H)⁺.

### Synthesis of tert-butyl 2-(4-(7-bromo-6-(difluoromethoxy)benzo[d]imidazo[2,1-b]thiazol-2-yl)phenyl)pyrrolidine-1-carboxylate (7, Scheme 67)

Compound tert-butyl 2-(4-(7-bromo-6-(difluoromethoxy)benzo[d]imidazo[2,1-b]thiazol-2-yl)phenyl)pyrrolidine-1-carboxylate was prepared from 6-bromo-5-(difluoromethoxy)benzo[d]thiazol-2-amine (0.50 g, 1.69 mmol) and tert-butyl 2-(4-(2-bromoacetyl)phenyl)pyrrolidine-1-carboxylate (0.94 g, 2.55 mmol) following a similar procedure to that described for the synthesis of tert-butyl (S)-2-(4-(7-bromo-9-methyl-9H-benzo[d]imidazo[1,2-a]imidazol-2-yl)-3-fluorophenyl)pyrrolidine-1-carboxylate, and was isolated as a white solid.

Yield 0.35 g (37%). ¹H NMR (400 MHz, DMSO) δ 8.79 (s, 1H), 8.51 (s, 1H), 8.10 (s, 1H), 7.79 (d, J=8.0 Hz, 2H), 7.34 (t, J=72.8 Hz, 1H), 7.25 (d, J=8.0 Hz, 2H), 4.90-4.66 (m, 1H), 3.62-3.43 (m, 2H), 2.39-2.21 (m, 1H), 1.91-1.65 (m, 3H), 1.41 (s, 3H), 1.12 (s, 6H). m/z: [ESI⁺] 564, 566 (M+H)⁺.

### Synthesis of tert-butyl 2-(4-(6-(difluoromethoxy)-7-((3-(4-fluoropiperidin-1-yl)propyl)carbamoyl)benzo[d]imidazo[2,1-b]thiazol-2-yl)phenyl)pyrrolidine-1-carboxylate (9, Scheme 67)

Compound tert-butyl 2-(4-(6-(difluoromethoxy)-7-((3-(4-fluoropiperidin-1-yl)propyl)carbamoyl)benzo[d]imidazo[2,1-b]thiazol-2-yl)phenyl)pyrrolidine-1-carboxylate was prepared from tert-butyl 2-(4-(7-bromo-6-(difluoromethoxy)benzo[d]imidazo[2,1-b]thiazol-2-yl)phenyl)pyrrolidine-1-carboxylate (300 mg, 0.531 mmol) and 3-(4-fluoropiperidin-1-yl)propan-1-amine (341 mg, 2.128 mmol) following a similar procedure to that described for the synthesis of tert-butyl (S)-2-(3-fluoro-4-(7-((3-(4-fluoropiperidin-1-yl)propyl)carbamoyl)-9-methyl-9H-benzo[d]imidazo[1,2-a]imidazol-2-yl)phenyl)pyrrolidine-1-carboxylate, and was isolated as a brown solid.

Yield 70 mg (20%). ¹H NMR (400 MHz, CDCl₃) δ 8.37 (s, 1H), 7.99 (s, 1H), 7.97-7.89 (br s, 1H), 7.83 (d, J=8.0 Hz, 2H), 7.47 (s, 1H), 7.28 (t, J=72.8 Hz, 1H), 7.26 (d, J=8.0 Hz, 2H), 5.05-4.54 (m, 2H), 3.78-3.55 (m, 6H), 2.76-2.23 (m, 7H), 2.06-1.73 (m, 7H), 1.49-1.22 (m, 9H). m/z: [ESI⁺] 672 (M+H)⁺.

### Synthesis of tert-butyl 2-(3-fluoro-4-(7-((3-(4-fluoropiperidin-1-yl)propyl)carbamoyl)-6-methoxybenzo[d]imidazo[2,1-b]thiazol-2-yl)phenyl)pyrrolidine-1-carboxylate (6)

### Synthesis of tert-butyl (R)-2-(3-fluoro-4-(7-((3-(4-fluoropiperidin-1-yl)propyl)carbamoyl)-6-methoxybenzo[d]imidazo[2,1-b]thiazol-2-yl)phenyl)pyrrolidine-1-carboxylate (7)

### Synthesis of tert-butyl (S)-2-(3-fluoro-4-(7-((3-(4-fluoropiperidin-1-yl)propyl)carbamoyl)-6-methoxybenzo[d]imidazo[2,1-b]thiazol-2-yl)phenyl)pyrrolidine-1-carboxylate (8)

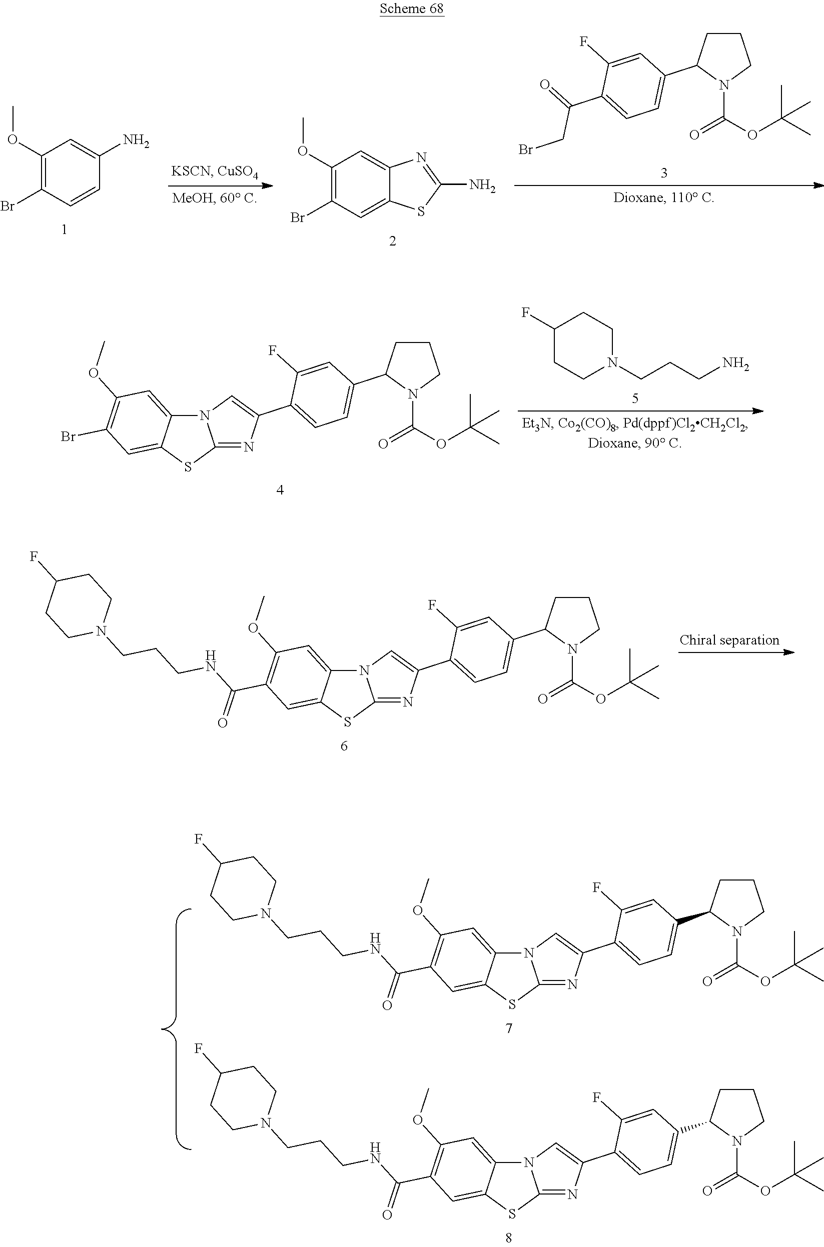

### Synthesis of 6-bromo-5-methoxybenzo[d]thiazol-2-amine (2, Scheme 68)

Compound 6-bromo-5-methoxybenzo[d]thiazol-2-amine was prepared from 4-bromo-3-methoxyaniline (2.00 g, 9.90 mmol) and potassium thiocyanate (9.62 g, 98.98 mmol) following a similar procedure to that described for the synthesis of 6-bromo-5-(2-methoxyethoxy)benzo[d]thiazol-2-amine, and was isolated as a yellow solid.

Yield 1.40 g (55%). ¹H NMR (400 MHz, DMSO) δ 7.84 (s, 1H), 7.58 (br s, 2H), 7.06 (s, 1H), 3.83 (s, 3H). m/z: [ESI⁺] 259, 261 (M+H)⁺.

### Synthesis of tert-butyl 2-(4-(7-bromo-6-methoxybenzo[d]imidazo[2,1-b]thiazol-2-yl)-3-fluorophenyl)pyrrolidine-1-carboxylate (4, Scheme 68)

Compound tert-butyl 2-(4-(7-bromo-6-methoxybenzo[d]imidazo[2,1-b]thiazol-2-yl)-3-fluorophenyl)pyrrolidine-1-carboxylate was prepared from 6-bromo-5-methoxybenzo[d]thiazol-2-amine (1.00 g, 3.86 mmol) and tert-butyl 2-(4-(2-bromoacetyl)-3-fluorophenyl)pyrrolidine-1-carboxylate (1.49 g, 3.86 mmol) following a similar procedure to that described for the synthesis of tert-butyl (S)-2-(4-(7-bromo-9-methyl-9H-benzo[d]imidazo[1,2-a]imidazol-2-yl)-3-fluorophenyl)pyrrolidine-1-carboxylate at 110° C., and was isolated as a brown solid.

Yield 0.80 g (38%). ¹H NMR (400 MHz, DMSO) δ 8.72 (s, 1H), 8.31 (s, 1H), 8.12-8.05 (m, 2H), 7.19-7.06 (m, 2H), 4.91-4.69 (m, 1H), 4.00 (s, 3H), 3.63-3.41 (m, 2H), 2.40-2.21 (m, 1H), 1.95-1.71 (m, 3H), 1.41-1.13 (m, 9H). m/z: [ESI⁺] 546, 548 (M+H)⁺.

### Synthesis of tert-butyl 2-(3-fluoro-4-(7-((3-(4-fluoropiperidin-1-yl)propyl)carbamoyl)-6-methoxybenzo[d]imidazo[2,1-b]thiazol-2-yl)phenyl)pyrrolidine-1-carboxylate (6, Scheme 68)

Compound tert-butyl 2-(3-fluoro-4-(7-((3-(4-fluoropiperidin-1-yl)propyl)carbamoyl)-6-methoxybenzo[d]imidazo[2,1-b]thiazol-2-yl)phenyl)pyrrolidine-1-carboxylate was prepared from tert-butyl 2-(4-(7-bromo-6-methoxybenzo[d]imidazo[2,1-b]thiazol-2-yl)-3-fluorophenyl)pyrrolidine-1-carboxylate (200 mg, 0.366 mmol) and 3-(4-fluoropiperidin-1-yl)propan-1-amine (120 mg, 0.749 mmol) following a similar procedure to that described for the synthesis of tert-butyl (S)-2-(3-fluoro-4-(7-((3-(4-fluoropiperidin-1-yl)propyl)carbamoyl)-9-methyl-9H-benzo[d]imidazo[1,2-a]imidazol-2-yl)phenyl)pyrrolidine-1-carboxylate, and was isolated as a yellow oil.

Yield 60 mg (25%). ¹H NMR (400 MHz, CDCl₃) δ 8.56 (s, 1H), 8.22-8.10 (m, 2H), 7.28 (d, J=1.6 Hz, 1H), 7.23 (s, 1H), 7.10 (dd, J=1.6, 8.0 Hz, 1H), 7.00 (d, J=8.0 Hz 1H), 5.03-4.76 (m, 2H), 4.17 (s, 3H), 3.74-3.54 (m, 5H), 2.44-2.29 (m, 1H), 2.03-1.82 (m, 6H), 1.69-1.52 (m, 8H), 1.49-1.25 (m, 9H). m/z: [ESI⁺] 654 (M+H)⁺.

### Synthesis of tert-butyl (R)-2-(3-fluoro-4-(7-((3-(4-fluoropiperidin-1-yl)propyl)carbamoyl)-6-methoxybenzo[d]imidazo[2,1-b]thiazol-2-yl)phenyl)pyrrolidine-1-carboxylate and tert-butyl (S)-2-(3-fluoro-4-(7-((3-(4-fluoropiperidin-1-yl)propyl)carbamoyl)-6-methoxybenzo[d]imidazo[2,1-b]thiazol-2-yl)phenyl)pyrrolidine-1-carboxylate (7, 8, Scheme 68)

tert-butyl 2-(3-fluoro-4-(7-((3-(4-fluoropiperidin-1-yl)propyl)carbamoyl)-6-methoxybenzo[d]imidazo[2,1-b]thiazol-2-yl)phenyl)pyrrolidine-1-carboxylate (100 mg, 0.153 mmol) was separated by CHIRAL-HPLC with the following conditions; Column: CHIRAL ART Cellulose-SC, 2×25 cm, 5 μm; Mobile Phase A: hexane (plus 0.5% 2 N ammonium-methanol), Mobile Phase B: ethanol/dichloromethane=1:1; Flow rate: 20 mL/min; Gradient: isocratic 40% B in 16 min; Detector: UV 254/220 nm. The faster eluting peak at 11.77 min was collected and concentrated under reduce pressure to afford tert-butyl (R)-2-(3-fluoro-4-(7-((3-(4-fluoropiperidin-1-yl)propyl)carbamoyl)-6-methoxybenzo[d]imidazo[2,1-b]thiazol-2-yl)phenyl)pyrrolidine-1-carboxylate as a light yellow solid.

Yield 30 mg (30%). ¹H NMR (400 MHz, DMSO) δ 8.74 (s, 1H), 8.37-8.23 (m, 2H), 8.14-8.03 (m, 2H), 7.18-7.07 (m, 2H), 4.89-4.57 (m, 2H), 4.02 (s, 3H), 3.61-3.42 (m, 2H), 2.40-2.23 (m, 3H), 1.97-1.65 (m, 15H), 1.41-1.14 (m, 9H). m/z: [ESI⁺] 654 (M+H)⁺.

The slower eluting peak at 14.43 min was collected and concentrated under reduce pressure to afford tert-butyl (S)-2-(3-fluoro-4-(7-((3-(4-fluoropiperidin-1-yl)propyl)carbamoyl)-6-methoxybenzo[d]imidazo[2,1-b]thiazol-2-yl)phenyl)pyrrolidine-1-carboxylate as a light yellow solid.

Yield 30 mg (30%). ¹H NMR (400 MHz, DMSO) δ 8.74 (s, 1H), 8.37-8.23 (m, 2H), 8.14-8.03 (m, 2H), 7.18-7.07 (m, 2H), 4.89-4.57 (m, 2H), 4.02 (s, 3H), 3.61-3.42 (m, 2H), 2.40-2.23 (m, 3H), 1.97-1.65 (m, 15H), 1.41-1.14 (m, 9H). m/z: [ESI⁺] 654 (M+H)⁺.

### Synthesis of 2-bromo-N-(3-(4-fluoropiperidin-1-yl)propyl)-6-methoxybenzo[d]imidazo[2,1-b]thiazole-7-carboxamide

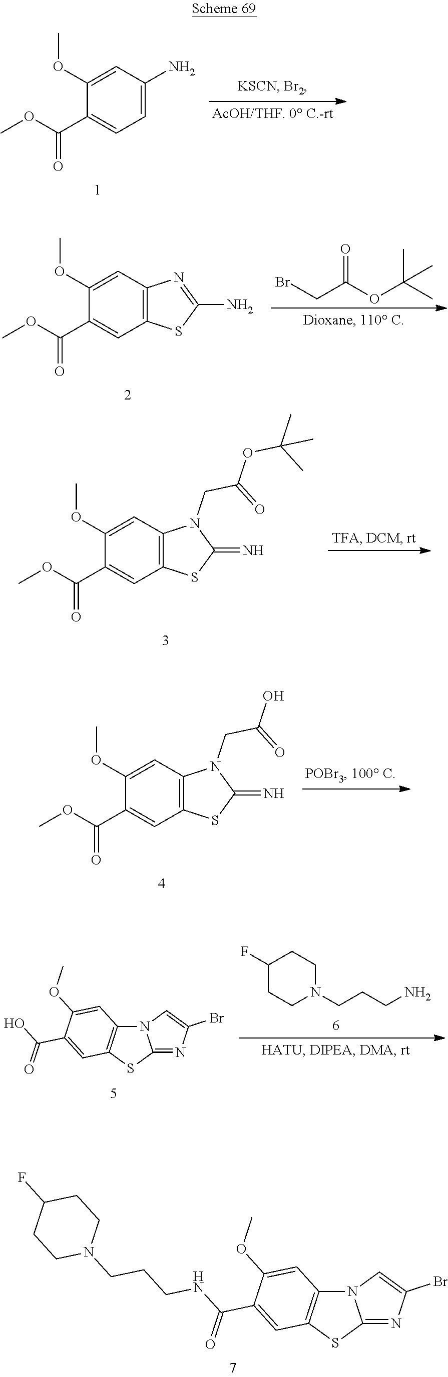

### Synthesis of methyl 2-amino-5-methoxybenzo[d]thiazole-6-carboxylate (2, Scheme 69)

To a stirred solution of methyl 4-amino-2-methoxybenzoate (50.00 g, 275.95 mmol) and potassium thiocyanate (53.63 g, 551.86 mmol) in acetic acid:tetrahydrofuran (2:1, 750 mL) was added bromine (14.14 mL, 275.95 mmol) dropwise over 5 min at 0° C. The reaction solution was stirred for 2 h at room temperature. To the above mixture was added an aqueous solution of saturated sodium thiosulfate (2000 mL). The precipitated solids were collected by filtration, washed with acetonitrile (3×500 mL) and dried in a vacuum oven to afford 2-amino-5-methoxybenzo[d]thiazole-6-carboxylate as an off-white solid.

Yield 48.55 g (74%). ¹H NMR (400 MHz, DMSO) δ 7.87 (s, 1H), 6.67 (br s, 2H), 6.47 (s, 1H), 3.77 (s, 3H), 3.71 (s, 3H). m/z: [ESI⁺] 239 (M+H)⁺.

### Synthesis of methyl 3-(2-(tert-butoxy)-2-oxoethyl)-2-imino-5-methoxy-2, 3-dihydrobenzo[d]thiazole-6-carboxylate (3, Scheme 69)

To a stirred solution of 2-amino-5-methoxybenzo[d]thiazole-6-carboxylate (90.00 g, 377.74 mmol) in 1,4-dioxane (1000 mL) was added tert-butyl 2-bromoacetate (88.42 g, 453.30 mmol) at room temperature. The reaction mixture was stirred for 16 h at 110° C. The mixture was allowed to cool to room temperature. The precipitated solids were collected by filtration, washed with methanol (3×300 mL) and dried under reduced pressure to afford methyl 3-(2-(tert-butoxy)-2-oxoethyl)-2-imino-5-methoxy-2,3-dihydrobenzo[d]thiazole-6-carboxylate as a light yellow solid.

Yield 77.00 g (58%). ¹H NMR (300 MHz, CDCl₃) δ 10.68 (s, 1H), 8.11 (s, 1H), 6.76 (s, 1H), 5.45 (s, 2H), 4.00 (s, 3H), 3.93 (s, 3H), 1.52 (s, 9H). m/z: [ESI⁺] 353 (M+H)⁺.

### Synthesis of 2-(2-imino-5-methoxy-6-(methoxycarbonyl)benzo[d]thiazol-3(2H)-yl)acetic Acid (4, Scheme 69)

To a stirred solution of methyl 3-(2-(tert-butoxy)-2-oxoethyl)-2-imino-5-methoxy-2,3-dihydrobenzo[d]thiazole-6-carboxylate (3.50 g, 9.93 mmol) in dichloromethane (30 mL) was added 2,2,2-trifluoroacetic acid (10 mL) dropwise at room temperature under a nitrogen atmosphere. The resulting solution was stirred for 2 h. The precipitated solids were collected by filtration, washed with diethyl ether (2×30 mL) and dried under reduced pressure to afford 2-(2-imino-5-methoxy-6-(methoxycarbonyl)benzo[d]thiazol-3(2H)-yl)acetic acid as a white solid.

Yield 2.50 g (85%). ¹H NMR (300 MHz, CDCl₃) δ 10.68 (s, 1H), 8.11 (s, 1H), 6.76 (s, 1H), 5.45 (s, 2H), 4.00 (s, 3H), 3.93 (s, 3H). OH proton not observed. m/z: [ESI⁺] 297, 299 (M+H)⁺.

### Synthesis of 2-bromo-6-methoxybenzo[d]imidazo[2,1-b]thiazole-7-carboxylic Acid (5, Scheme 69)

A mixture of 2-(2-imino-5-methoxy-6-(methoxycarbonyl)benzo[d]thiazol-3(2H)-yl)acetic acid (2.50 g, 8.44 mmol) and phosphoryl tribromide (20.00 g, 69.76 mmol) was stirred for 3 h at 100° C. under a nitrogen atmosphere. The resulting mixture was cooled to room temperature and diluted with ethyl acetate (100 mL). The precipitated solids were collected by filtration and washed sequentially with ethyl acetate (3×10 mL) and water (3×10 mL). The residue was purified by reverse phase flash column chromatography with the following conditions; Column: WelFlash™ C18-I, 20-40 μm, 330 g; Eluent A: water (plus 10 mmol/L ammonium bicarbonate); Eluent B: acetonitrile; Gradient: 45%-65% B in 25 min; Flow rate: 80 mL/min; Detector: UV 220/254 nm. The desired fractions were collected at 62% B and concentrated under reduced pressure to afford 2-bromo-6-methoxybenzo[d]imidazo[2,1-b]thiazole-7-carboxylic acid as a white solid.

Yield 0.50 g (18%). ¹H NMR (400 MHz, DMSO) δ 8.56 (s, 1H), 8.38 (s, 1H), 7.90 (s, 1H), 3.94 (s, 3H).

OH proton not observed. m/z: [ESI⁺] 327, 329 (M+H)⁺.

### Synthesis of 2-bromo-N-(3-(4-fluoropiperidin-1-yl)propyl)-6-methoxybenzo[d]imidazo[2,1-b]thiazole-7-carboxamide (7, Scheme 69)

Compound 2-bromo-N-(3-(4-fluoropiperidin-1-yl)propyl)-6-methoxybenzo[d]imidazo[2,1-b]thiazole-7-carboxamide was prepared from 2-bromo-6-methoxybenzo[d]imidazo[2,1-b]thiazole-7-carboxylic acid (300 mg, 0.917 mmol) and 3-(4-fluoropiperidin-1-yl)propan-1-amine (220 mg, 1.373 mmol) following a similar procedure to that described for the synthesis of tert-butyl 6-fluoro-5-(7-((3-(4-fluoropiperidin-1-yl)propyl)carbamoyl)benzo[d]imidazo[2,1-b]thiazol-2-yl)indoline-1-carboxylate, and was isolated as a white solid.

Yield 150 mg (35%). ¹H NMR (400 MHz, CD₃OD) δ 8.32 (s, 1H), 8.23 (s, 1H), 7.73 (s, 1H), 4.80-4.60 (m, 1H), 4.10 (s, 3H), 3.55-3.46 (m, 2H), 2.73-2.60 (m, 2H), 2.56-2.40 (m, 4H), 1.94-1.77 (m, 6H). NH proton not observed. m/z: [ESI⁺] 469, 471 (M+H)⁺.

### Synthesis of tert-butyl 2-(4-(7-carbamoyl-6-methoxybenzo[d]imidazo[2,1-b]thiazol-2-yl)-3-fluorophenyl)pyrrolidine-1-carboxylate

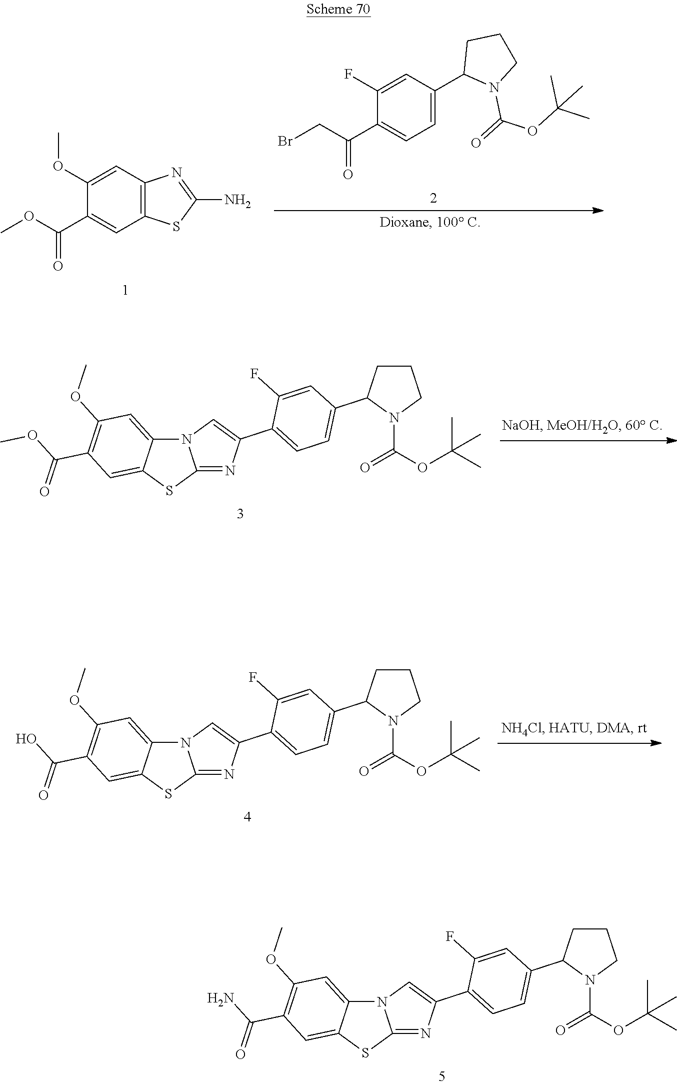 [SMILES: COC(=O)c1cc2sc(N)nc2cc1OC ; CC(C)(C)OC(=O)N1CCCC1c1ccc(C(=O)CBr)c(F)c1 ; COC(=O)c1cc2sc3nc(-c4ccc(C5CCCN5C(=O)OC(C)(C)C)cc4F)cn3c2cc1OC ; COc1cc2c(cc1C(=O)O)sc1nc(-c3ccc(C4CCCN4C(=O)OC(C)(C)C)cc3F)cn12 ; COc1cc2c(cc1C(N)=O)sc1nc(-c3ccc(C4CCCN4C(=O)OC(C)(C)C)cc3F)cn12]

### Synthesis of methyl 2-(4-(1-(tert-butoxycarbonyl)pyrrolidin-2-yl)-2-fluorophenyl)-6-methoxybenzo[d]imidazo[2,1-b]thiazole-7-carboxylate (3, Scheme 70)

Compound methyl 2-(4-(1-(tert-butoxycarbonyl)pyrrolidin-2-yl)-2-fluorophenyl)-6-methoxybenzo[d]imidazo[2,1-b]thiazole-7-carboxylate was prepared from methyl 2-amino-5-methoxybenzo[d]thiazole-6-carboxylate (1.00 g, 4.20 mmol) and tert-butyl 2-(4-(2-bromoacetyl)-3-fluorophenyl)pyrrolidine-1-carboxylate (1.63 g, 4.22 mmol) following a similar procedure to that described for the synthesis of tert-butyl (S)-2-(4-(7-bromo-9-methyl-9H-benzo[d]imidazo[1,2-a]imidazol-2-yl)-3-fluorophenyl)pyrrolidine-1-carboxylate at 110° C., and was isolated as a light yellow solid.

Yield 0.55 g (25%). ¹H NMR (400 MHz, CDCl₃) δ 8.22 (s, 1H), 8.20-8.11 (m, 1H), 7.28 (s, 1H), 7.23 (s, 1H), 7.10 (dd, J=1.6, 8.0 Hz, 1H), 7.00 (dd, J=1.6, 12.4 Hz, 1H), 4.89-4.78 (m, 1H), 4.07 (s, 3H), 3.96 (s, 3H), 3.70-3.59 (m, 2H), 2.43-2.27 (m, 1H), 2.01-1.81 (m, 3H), 1.49-1.25 (m, 9H). m/z: [ESI⁺]526 (M+H)⁺.

### Synthesis of 2-(4-(1-(tert-butoxycarbonyl)pyrrolidin-2-yl)-2-fluorophenyl)-6-methoxybenzo[d]imidazo[2,1-b]thiazole-7-carboxylic Acid (4, Scheme 70)

Compound 2-(4-(1-(tert-butoxycarbonyl)pyrrolidin-2-yl)-2-fluorophenyl)-6-methoxybenzo[d]imidazo[2,1-b]thiazole-7-carboxylic acid was prepared from methyl 2-(4-(1-(tert-butoxycarbonyl)pyrrolidin-2-yl)-2-fluorophenyl)-6-methoxybenzo[d]imidazo[2,1-b]thiazole-7-carboxylate (0.55 g, 1.05 mmol) following a similar procedure to that described for the synthesis of 2-(1-(tert-butoxycarbonyl)-6-fluoroindolin-5-yl)benzo[d]imidazo[2,1-b]thiazole-7-carboxylic acid at room temperature, and was isolated as a light yellow solid.

Yield 0.45 g (84%). ¹H NMR (400 MHz, CDCl₃) δ 8.56 (s, 1H), 8.24-8.12 (m, 2H), 7.31-7.29 (m, 1H), 7.11 (dd, J=1.6, 8.0 Hz, 1H), 7.01 (dd, J=1.6, 12.4 Hz, 1H), 5.05-4.77 (m, 1H), 4.25 (s, 3H), 3.75-3.60 (m, 2H), 2.45-2.27 (m, 1H), 2.03-1.84 (m, 3H), 1.50-1.25 (m, 9H). OH proton not observed. m/z: [ESI⁺] 512 (M+H)⁺.

### Synthesis of tert-butyl 2-(4-(7-carbamoyl-6-methoxybenzo[d]imidazo[2,1-b]thiazol-2-yl)-3-fluorophenyl)pyrrolidine-1-carboxylate (5, Scheme 70)

Compound tert-butyl 2-(4-(7-carbamoyl-6-methoxybenzo[d]imidazo[2,1-b]thiazol-2-yl)-3-fluorophenyl)pyrrolidine-1-carboxylate was prepared from 2-(4-(1-(tert-butoxycarbonyl)pyrrolidin-2-yl)-2-fluorophenyl)-6-methoxybenzo[d]imidazo[2,1-b]thiazole-7-carboxylic acid (0.50 g, 0.98 mmol) and ammonium chloride (0.20 g, 3.74 mmol) following a similar procedure to that described for the synthesis of tert-butyl 6-fluoro-5-(7-((3-(4-fluoropiperidin-1-yl)propyl)carbamoyl)benzo[d]imidazo[2,1-b]thiazol-2-yl)indoline-1-carboxylate, and was isolated as a brown solid.

Yield 0.20 g (40%). ¹H NMR (400 MHz, DMSO) δ 8.75 (d, J=3.6 Hz, 1H), 8.42 (s, 1H), 8.14-8.05 (m, 2H), 7.75 (s, 1H), 7.66 (s, 1H), 7.17-7.09 (m, 2H), 4.92-4.71 (m, 1H), 4.04 (s, 3H), 3.63-3.43 (m, 2H), 2.39-2.23 (m, 1H), 1.92-1.69 (m, 3H), 1.41-1.14 (m, 9H). m/z: [ESI⁺] 511 (M+H)⁺.

### Synthesis of tert-butyl 2-(3-fluoro-4-(6-methoxy-7-(methylcarbamoyl)benzo[d]imidazo[2,1-b]thiazol-2-yl)phenyl)pyrrolidine-1-carboxylate

Compound tert-butyl 2-(3-fluoro-4-(6-methoxy-7-(methylcarbamoyl)benzo[d]imidazo[2,1-b]thiazol-2-yl)phenyl)pyrrolidine-1-carboxylate was prepared from 2-(4-(1-(tert-butoxycarbonyl)pyrrolidin-2-yl)-2-fluorophenyl)-6-methoxybenzo[d]imidazo[2,1-b]thiazole-7-carboxylic acid (40 mg, 0.078 mmol) and methylamine (5 mg, 0.161 mmol) following a similar procedure to that described for the synthesis of tert-butyl 6-fluoro-5-(7-((3-(4-fluoropiperidin-1-yl)propyl)carbamoyl)benzo[d]imidazo[2,1-b]thiazol-2-yl)indoline-1-carboxylate, and was isolated as a light yellow solid.

Yield 25 mg (61%). ¹H NMR (400 MHz, DMSO) δ 8.75 (d, J=3.6 Hz, 1H), 8.37 (s, 1H), 8.25 (q, J=4.8 Hz, 1H), 8.14-8.03 (m, 2H), 7.19-7.07 (m, 2H), 4.91-4.68 (m, 1H), 4.03 (s, 3H), 3.63-3.43 (m, 2H), 2.84 (d, J=4.8 Hz, 3H), 2.39-2.23 (m, 1H), 1.92-1.68 (m, 3H), 1.41-1.14 (m, 9H). m/z: [ESI⁺] 525 (M+H)⁺.

### Synthesis of tert-butyl 2-(3-fluoro-4-(6-methoxy-7-((1-methylpiperidin-4-yl)carbamoyl)benzo[d]imidazo[2,1-b]thiazol-2-yl)phenyl)pyrrolidine-1-carboxylate

Compound tert-butyl 2-(3-fluoro-4-(6-methoxy-7-((1-methylpiperidin-4-yl)carbamoyl)benzo[d]imidazo[2,1-b]thiazol-2-yl)phenyl)pyrrolidine-1-carboxylate was prepared from 2-(4-(1-(tert-butoxycarbonyl)pyrrolidin-2-yl)-2-fluorophenyl)-6-methoxybenzo[d]imidazo[2,1-b]thiazole-7-carboxylic acid (150 mg, 0.293 mmol) and 1-methylpiperidin-4-amine (70 mg, 0.613 mmol) following a similar procedure to that described for the synthesis of tert-butyl 6-fluoro-5-(7-((3-(4-fluoropiperidin-1-yl)propyl)carbamoyl)benzo[d]imidazo[2,1-b]thiazol-2-yl)indoline-1-carboxylate, and was isolated as a yellow solid.

Yield 100 mg (56%). ¹H NMR (400 MHz, DMSO) δ 8.74 (d, J=3.6 Hz, 1H), 8.28 (s, 1H), 8.12-8.03 (m, 3H), 7.18-7.09 (m, 2H), 4.88-4.70 (m, 1H), 4.02 (s, 3H), 3.83-3.71 (m, 1H), 3.63-3.42 (m, 2H), 2.74-2.65 (m, 2H), 2.40-2.26 (m, 1H), 2.18 (s, 3H), 2.09-1.98 (m, 2H), 1.91-1.72 (m, 5H), 1.65-1.50 (m, 2H), 1.41-1.13 (m, 9H). m/z: [ESI⁺] 608 (M+H)⁺.

### Synthesis of tert-butyl 2-(3-fluoro-4-(6-methoxy-7-(((R)-1-methylpiperidin-3-yl)carbamoyl)benzo[d]imidazo[2,1-b]thiazol-2-yl)phenyl)pyrrolidine-1-carboxylate

Compound tert-butyl 2-(3-fluoro-4-(6-methoxy-7-(((R)-1-methylpiperidin-3-yl)carbamoyl)benzo[d]imidazo[2,1-b]thiazol-2-yl)phenyl)pyrrolidine-1-carboxylate was prepared from 2-(4-(1-(tert-butoxycarbonyl)pyrrolidin-2-yl)-2-fluorophenyl)-6-methoxybenzo[d]imidazo[2,1-b]thiazole-7-carboxylic acid (100 mg, 0.195 mmol) and (R)-1-methylpiperidin-3-amine (26 mg, 0.228 mmol) following a similar procedure to that described for the synthesis of tert-butyl 6-fluoro-5-(7-((3-(4-fluoropiperidin-1-yl)propyl)carbamoyl)benzo[d]imidazo[2,1-b]thiazol-2-yl)indoline-1-carboxylate, and was isolated as a light yellow solid.

Yield 50 mg (42%). ¹H NMR (400 MHz, DMSO) δ 8.75 (d, J=3.6 Hz, 1H), 8.36-8.30 (m, 1H), 8.26 (s, 1H), 8.14-8.05 (m, 2H), 7.17-7.10 (m, 2H), 4.91-4.67 (m, 1H), 4.01 (s, 3H), 3.68-3.28 (m, 4H), 2.89-2.74 (m, 4H), 2.70 (s, 3H), 2.42-2.23 (m, 1H), 2.01-1.72 (m, 6H), 1.41-1.13 (m, 9H). m/z: [ESI⁺] 608 (M+H)⁺.

### Synthesis of tert-butyl 2-(3-fluoro-4-(6-methoxy-7-(((S)-1-methylpiperidin-3-yl)carbamoyl)benzo[d]imidazo[2,1-b]thiazol-2-yl)phenyl)pyrrolidine-1-carboxylate

Compound tert-butyl 2-(3-fluoro-4-(6-methoxy-7-(((S)-1-methylpiperidin-3-yl)carbamoyl)benzo[d]imidazo[2,1-b]thiazol-2-yl)phenyl)pyrrolidine-1-carboxylate was prepared from 2-(4-(1-(tert-butoxycarbonyl)pyrrolidin-2-yl)-2-fluorophenyl)-6-methoxybenzo[d]imidazo[2,1-b]thiazole-7-carboxylic acid (200 mg, 0.391 mmol) and (S)-1-methylpiperidin-3-amine (54 mg, 0.473 mmol) following a similar procedure to that described for the synthesis of tert-butyl 6-fluoro-5-(7-((3-(4-fluoropiperidin-1-yl)propyl)carbamoyl)benzo[d]imidazo[2,1-b]thiazol-2-yl)indoline-1-carboxylate, and was isolated as a light yellow solid.

Yield 100 mg (42%). ¹H NMR (400 MHz, DMSO) δ 8.75 (d, J=3.6 Hz, 1H), 8.36-8.30 (m, 1H), 8.26 (s, 1H), 8.14-8.05 (m, 2H), 7.17-7.10 (m, 2H), 4.91-4.67 (m, 1H), 4.01 (s, 3H), 3.68-3.28 (m, 4H), 2.89-2.74 (m, 4H), 2.70 (s, 3H), 2.42-2.23 (m, 1H), 2.01-1.72 (m, 6H), 1.41-1.13 (m, 9H). m/z: [ESI⁺] 608 (M+H)⁺.

### Synthesis of tert-butyl 2-(3-fluoro-4-(7-((3-(4-fluoropiperidin-1-yl)propyl)carbamoyl)-6-(2-methoxyethoxy)benzo[d]imidazo[2,1-b]thiazol-2-yl)phenyl)pyrrolidine-1-carboxylate

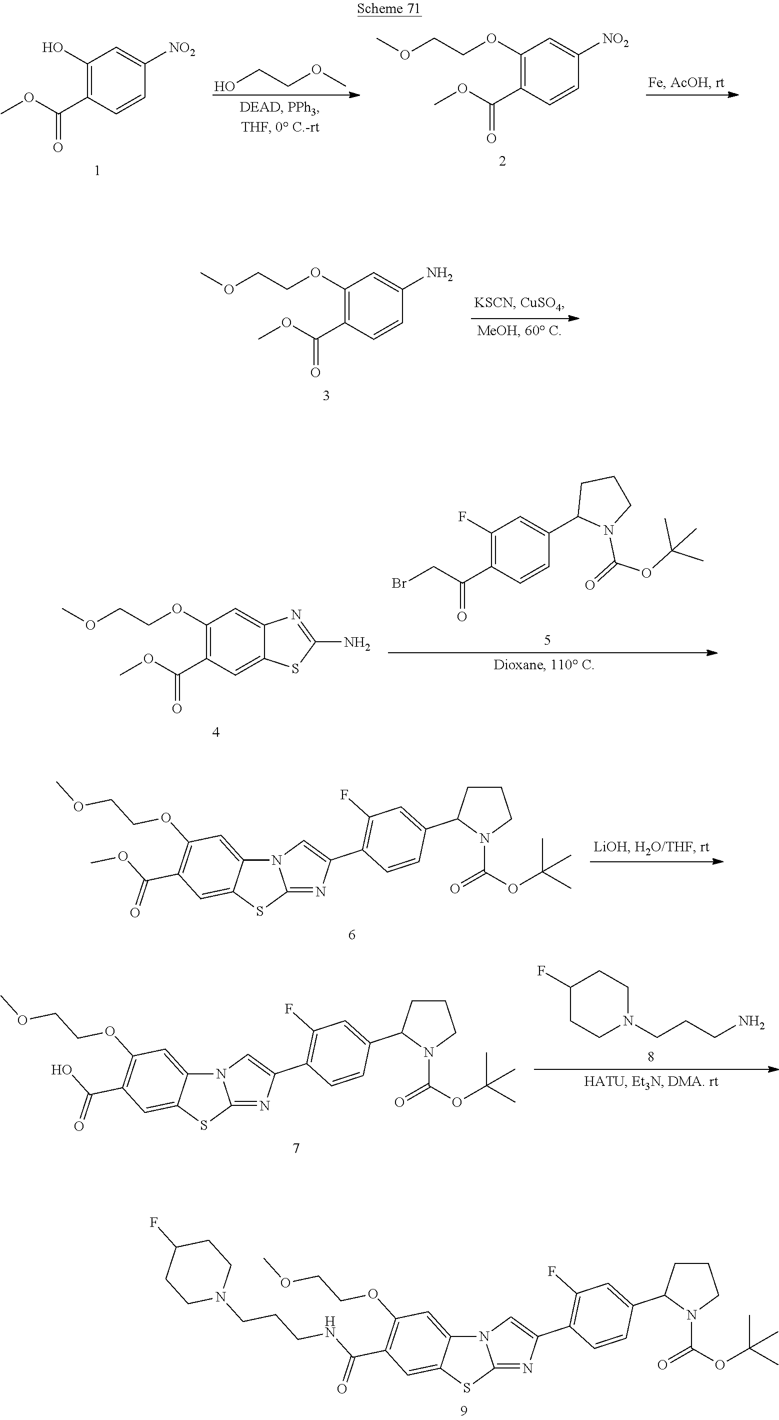

### Synthesis of methyl 2-(2-methoxyethoxy)-4-nitrobenzoate (2, Scheme 71)

Compound methyl 2-(2-methoxyethoxy)-4-nitrobenzoate was prepared from methyl 2-hydroxy-4-nitrobenzoate (2.00 g, 10.14 mmol) and 2-methoxyethan-1-ol (1.54 g, 20.24 mmol) following a similar procedure to that described for the synthesis of 1-bromo-2-(2-methoxyethoxy)-4-nitrobenzene, and was isolated as an orange oil.

Yield 2.50 g (97%). ¹H NMR (400 MHz, CDCl₃) δ 7.92 (d, J=8.4 Hz, 1H), 7.89-7.84 (m, 2H), 4.33-4.29 (m, 2H), 3.95 (s, 3H), 3.87-3.83 (m, 2H), 3.49 (s, 3H). No mass ion observed.

### Synthesis of methyl 4-amino-2-(2-methoxyethoxy)benzoate (3, Scheme 71)

Compound methyl 4-amino-2-(2-methoxyethoxy)benzoate was prepared from methyl 2-(2-methoxyethoxy)-4-nitrobenzoate (2.50 g, 9.80 mmol) following a similar procedure to that described for the synthesis of 4-bromo-3-(2-methoxyethoxy)aniline, and was isolated as an orange oil.

Yield 2.50 g (crude). ¹H NMR (400 MHz, CDCl₃) δ 7.75 (d, J=8.4 Hz, 1H), 6.28 (dd, J=2.0, 8.4 Hz, 1H), 6.25 (d, J=2.0 Hz, 1H), 4.14 (t, J=4.8 Hz, 2H), 3.83 (s, 3H), 3.82 (t, J=4.8 Hz, 2H), 3.50 (s, 3H). NH₂ protons not observed. m/z: [ESI⁺] 226 (M+H)⁺.

### Synthesis of methyl 2-amino-5-(2-methoxyethoxy)benzo[d]thiazole-6-carboxylate (4, Scheme 71)

Compound methyl 2-amino-5-(2-methoxyethoxy)benzo[d]thiazole-6-carboxylate was prepared from methyl 4-amino-2-(2-methoxyethoxy)benzoate (2.50 g, crude) and potassium thiocyanate (11.01 g, 113.29 mmol) following a similar procedure to that described for the synthesis of 6-bromo-5-(2-methoxyethoxy)benzo[d]thiazol-2-amine, and was isolated as tan solid.

Yield 1.80 g (57%). ¹H NMR (400 MHz, DMSO) δ 8.18 (br s, 2H), 8.05 (s, 1H), 7.07 (s, 1H), 4.19-4.13 (m, 2H), 3.76 (s, 3H), 3.72-3.66 (m, 2H), 3.35 (s, 3H). m/z: [ESI⁺] 283 (M+H)⁺.

### Synthesis of methyl 2-(4-(1-(tert-butoxycarbonyl)pyrrolidin-2-yl)-2-fluorophenyl)-6-(2-methoxyethoxy)benzo[d]imidazo[2,1-b]thiazole-7-carboxylate (6, Scheme 71)

Compound methyl 2-(4-(1-(tert-butoxycarbonyl)pyrrolidin-2-yl)-2-fluorophenyl)-6-(2-methoxyethoxy)benzo[d]imidazo[2,1-b]thiazole-7-carboxylate was prepared from methyl 2-amino-5-(2-methoxyethoxy)benzo[d]thiazole-6-carboxylate (0.80 g, 2.83 mmol) and tert-butyl 2-(4-(2-bromoacetyl)-3-fluorophenyl)pyrrolidine-1-carboxylate (1.20 g, 3.11 mmol) following a similar procedure to that described for the synthesis of tert-butyl (S)-2-(4-(7-bromo-9-methyl-9H-benzo[d]imidazo[1,2-a]imidazol-2-yl)-3-fluorophenyl)pyrrolidine-1-carboxylate at 110° C., and was isolated as a yellow solid.

Yield 0.30 g (19%). ¹H NMR (400 MHz, CDCl₃) δ 8.22 (s, 1H), 8.18 (dd, J=8.0, 8.0 Hz, 1H), 8.13 (s, 1H), 7.31 (s, 1H), 7.10 (dd, J=1.6, 8.0 Hz, 1H), 7.00 (dd, J=1.6, 12.4 Hz, 1H), 5.03-4.77 (m, 1H), 4.38-4.31 (m, 2H), 3.95 (s, 3H), 3.92-3.88 (m, 2H), 3.71-3.60 (m, 2H), 3.53 (s, 3H), 2.45-2.27 (m, 1H), 2.01-1.81 (m, 3H), 1.50-1.25 (m, 9H). m/z: [ESI⁺] 570 (M+H)⁺.

### Synthesis of 2-(4-(1-(tert-butoxycarbonyl)pyrrolidin-2-yl)-2-fluorophenyl)-6-(2-methoxyethoxy)benzo[d]imidazo[2,1-b]thiazole-7-carboxylic Acid (7, Scheme 71)

Compound 2-(4-(1-(tert-butoxycarbonyl)pyrrolidin-2-yl)-2-fluorophenyl)-6-(2-methoxyethoxy)benzo[d]imidazo[2,1-b]thiazole-7-carboxylic acid was prepared from methyl 2-(4-(1-(tert-butoxycarbonyl)pyrrolidin-2-yl)-2-fluorophenyl)-6-(2-methoxyethoxy)benzo[d]imidazo[2,1-b]thiazole-7-carboxylate (300 mg, 0.527 mmol) following a similar procedure to that described for the synthesis of 2-(1-(tert-butoxycarbonyl)-6-fluoroindolin-5-yl)benzo[d]imidazo[2,1-b]thiazole-7-carboxylic acid at 60° C., and was isolated as a yellow solid.

Yield 0.20 g (68%). ¹H NMR (400 MHz, CDCl₃) δ 8.31-8.06 (m, 2H), 8.02 (s, 1H), 7.28 (s, 1H), 7.13-7.00 (m, 1H), 7.00-6.93 (m, 1H), 5.02-4.74 (m, 1H), 4.37-4.24 (m, 2H), 3.89 (t, J=5.6 Hz, 2H), 3.66 (t, J=5.6 Hz, 2H), 3.47 (s, 3H), 2.43-2.29 (m, 1H), 2.03-1.85 (m, 3H), 1.50-1.25 (m, 9H). OH proton not observed. m/z: [ESI⁺] 556 (M+H)⁺.

### Synthesis of tert-butyl 2-(3-fluoro-4-(7-((3-(4-fluoropiperidin-1-yl)propyl)carbamoyl)-6-(2-methoxyethoxy)benzo[d]imidazo[2,1-b]thiazol-2-yl)phenyl)pyrrolidine-1-carboxylate (9, Scheme 71)

Compound tert-butyl 2-(3-fluoro-4-(7-((3-(4-fluoropiperidin-1-yl)propyl)carbamoyl)-6-(2-methoxyethoxy)benzo[d]imidazo[2,1-b]thiazol-2-yl)phenyl)pyrrolidine-1-carboxylate was prepared from 2-(4-(1-(tert-butoxycarbonyl)pyrrolidin-2-yl)-2-fluorophenyl)-6-(2-methoxyethoxy)benzo[d]imidazo[2,1-b]thiazole-7-carboxylic acid (200 mg, 0.360 mmol) and 3-(4-fluoropiperidin-1-yl)propan-1-amine (115 mg, 0.718 mmol) following a similar procedure to that described for the synthesis of tert-butyl 6-fluoro-5-(7-((3-(4-fluoropiperidin-1-yl)propyl)carbamoyl)benzo[d]imidazo[2,1-b]thiazol-2-yl)indoline-1-carboxylate, and was isolated as an orange oil.

Yield 105 mg (42%). ¹H NMR (400 MHz, CDCl₃) δ 8.57 (s, 1H), 8.29 (br s, 1H), 8.18 (dd, J=8.0, 8.0 Hz, 1H), 8.12 (s, 1H), 7.23 (s, 1H), 7.10 (dd, J=1.6, 8.0 Hz, 1H), 7.00 (d, J=12.4 Hz, 1H), 5.04-4.60 (m, 2H), 4.44-4.37 (m, 2H), 3.96-3.84 (m, 2H), 3.70-3.48 (m, 2H), 3.52 (s, 3H), 2.72-2.26 (m, 8H), 2.03-1.79 (m, 10H), 1.49-1.25 (m, 9H). m/z: [ESI⁺] 698 (M+H)⁺.

### Synthesis of 2-(2-fluoro-4-(5-oxopyrrolidin-2-yl)phenyl)-6-methoxybenzo[d]imidazo[2,1-b]thiazole-7-carboxylic Acid

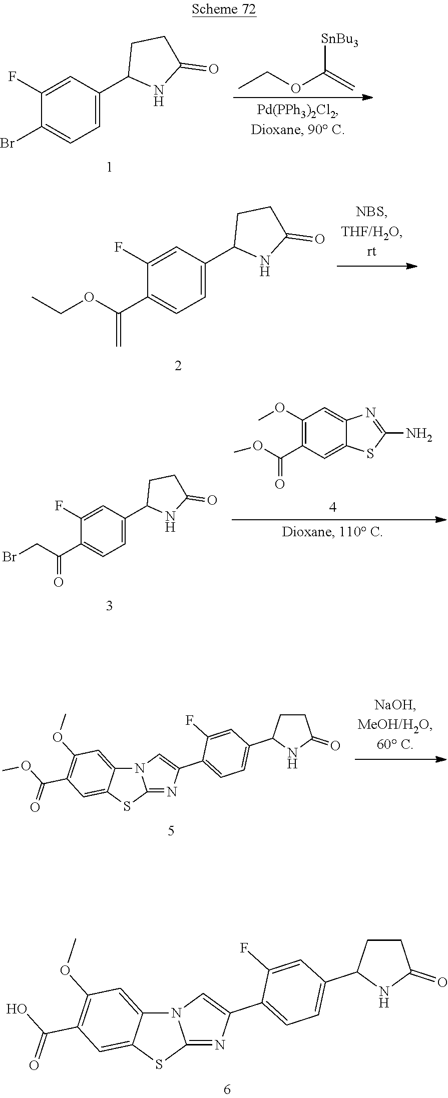

### Synthesis of 5-(4-(1-ethoxyvinyl)-3-fluorophenyl)pyrrolidin-2-one (2, Scheme 72)

Compound 5-(4-(1-ethoxyvinyl)-3-fluorophenyl)pyrrolidin-2-one was prepared from 5-(4-bromo-3-fluorophenyl)pyrrolidin-2-one (600 mg, 2.325 mmol) and tributyl(1-ethoxyvinyl)stannane (1.00 g, 2.77 mmol) following a similar procedure to that described for the synthesis of tert-butyl (S)-2-(4-(1-ethoxyvinyl)-3-fluorophenyl)pyrrolidine-1-carboxylate, and was isolated as a brown oil.

Yield 500 mg (crude). ¹H NMR not run. m/z: [ESI⁺] 250 (M+H)⁺.

### Synthesis of 5-(4-(2-bromoacetyl)-3-fluorophenyl)pyrrolidin-2-one (3, Scheme 72)

Compound 5-(4-(2-bromoacetyl)-3-fluorophenyl)pyrrolidin-2-one was prepared from 5-(4-(1-ethoxyvinyl)-3-fluorophenyl)pyrrolidin-2-one (500 mg, crude) and N-bromosuccinimide (370 mg, 2.079 mmol) following a similar procedure to that described for the synthesis of tert-butyl (S)-2-(4-(2-bromoacetyl)-3-fluorophenyl)pyrrolidine-1-carboxylate, and was isolated as a light yellow oil.

Yield 300 mg (crude). ¹H NMR not run. m/z: [ESI⁺] 300, 302 (M+H)⁺.

### Synthesis of methyl 2-(2-fluoro-4-(5-oxopyrrolidin-2-yl)phenyl)-6-methoxybenzo[d]imidazo[2,1-b]thiazole-7-carboxylate (5, Scheme 72)

Compound methyl 2-(2-fluoro-4-(5-oxopyrrolidin-2-yl)phenyl)-6-methoxybenzo[d]imidazo[2,1-b]thiazole-7-carboxylate was prepared from methyl 2-amino-5-methoxybenzo[d]thiazole-6-carboxylate (290 mg, 1.217 mmol) and 5-(4-(2-bromoacetyl)-3-fluorophenyl)pyrrolidin-2-one (330 mg, 1.100 mmol) following a similar procedure to that described for the synthesis of tert-butyl (S)-2-(4-(7-bromo-9-methyl-9H-benzo[d]imidazo[1,2-a]imidazol-2-yl)-3-fluorophenyl)pyrrolidine-1-carboxylate at 110° C., and was isolated as a brown solid.

Yield 70 mg (14% over three steps). ¹H NMR (300 MHz, DMSO) δ 8.82-8.73 (m, 1H), 8.40-8.33 (m, 1H), 8.20-8.06 (m, 3H), 7.34-7.20 (m, 2H), 4.79-4.67 (m, 1H), 3.97 (s, 3H), 3.84 (s, 3H), 2.57-2.53 (m, 1H), 2.32-2.21 (m, 2H), 1.91-1.74 (m, 1H). m/z: [ESI⁺] 440 (M+H)⁺.

### Synthesis of 2-(2-fluoro-4-(5-oxopyrrolidin-2-yl)phenyl)-6-methoxybenzo[d]imidazo[2,1-b]thiazole-7-carboxylic Acid (6, Scheme 72)

Compound 2-(2-fluoro-4-(5-oxopyrrolidin-2-yl)phenyl)-6-methoxybenzo[d]imidazo[2,1-b]thiazole-7-carboxylic acid was prepared from methyl 2-(2-fluoro-4-(5-oxopyrrolidin-2-yl)phenyl)-6-methoxybenzo[d]imidazo[2,1-b]thiazole-7-carboxylate (70 mg, 0.159 mmol) following a similar procedure to that described for the synthesis of 2-(1-(tert-butoxycarbonyl)-5-fluoroindolin-6-yl)benzo[d]imidazo[2,1-b]thiazole-7-carboxylic acid at 60° C., and was isolated as an off-white solid.

Yield 65 mg (96%). ¹H NMR (300 MHz, DMSO) δ 12.79 (br s, 1H), 8.81-8.71 (m, 1H), 8.48 (br s, 1H), 8.40-8.29 (m, 1H), 8.18-8.04 (m, 2H), 7.32-7.21 (m, 2H), 4.79-4.68 (m, 1H), 3.97 (s, 3H), 2.58-2.43 (m, 1H), 2.33-2.13 (m, 2H), 1.93-1.74 (m, 1H). m/z: [ESI⁺] 426 (M+H)⁺.

### Synthesis of 6-methoxy-2-(4-(methylcarbamoyl)phenyl)benzo[d]imidazo[2,1-b]thiazole-7-carboxylic Acid

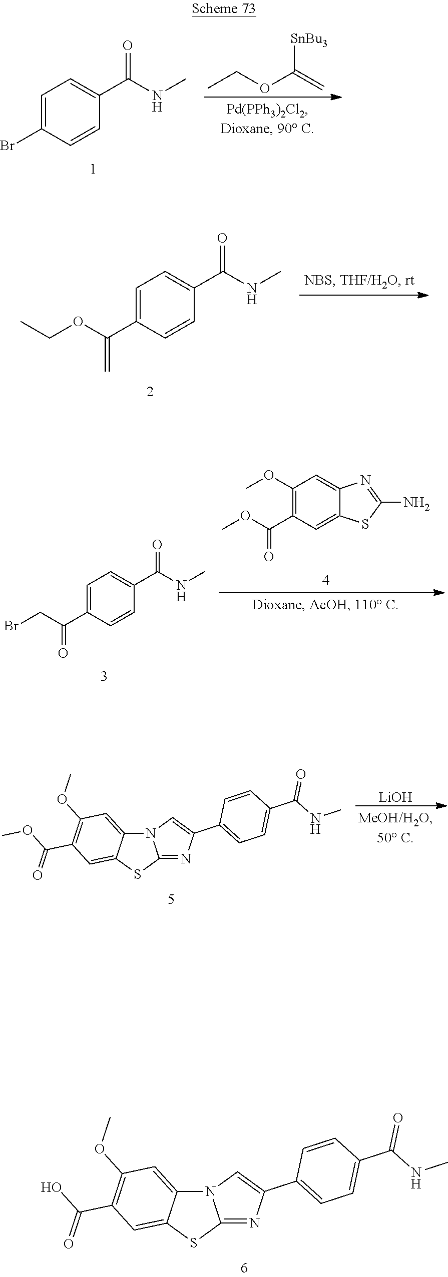

### Synthesis of 4-(1-ethoxyvinyl)-N-methylbenzamide (2, Scheme 73)

Compound 4-(1-ethoxyvinyl)-N-methylbenzamide was prepared from 4-bromo-N-methylbenzamide (3.00 g, 14.01 mmol) and tributyl(1-ethoxyvinyl)stannane (6.07 g, 16.81 mmol) following a similar procedure to that described for the synthesis of tert-butyl (S)-2-(4-(1-ethoxyvinyl)-3-fluorophenyl)pyrrolidine-1-carboxylate, and was isolated as a brown solid.

Yield 2.00 g (70%). ¹H NMR (400 MHz, DMSO) δ 8.45 (q, J=4.8 Hz, 1H), 7.83 (d, J=8.4 Hz, 2H), 7.69 (d, J=8.4 Hz, 2H), 4.87 (d, J=2.8 Hz, 1H), 4.37 (d, J=2.8 Hz, 1H), 3.91 (q, J=7.2 Hz, 2H), 2.79 (d, J=4.8 Hz, 3H), 1.37 (t, J=7.2 Hz, 3H). m/z: [ESI⁺] 206 (M+H)⁺.

### Synthesis of 4-(2-bromoacetyl)-N-methylbenzamide (3, Scheme 73)

Compound 4-(2-bromoacetyl)-N-methylbenzamide was prepared from 4-(1-ethoxyvinyl)-N-methylbenzamide (2.00 g, 9.74 mmol) and N-bromosuccinimide (1.91 g, 10.73 mmol) following a similar procedure to that described for the synthesis of tert-butyl (S)-2-(4-(2-bromoacetyl)-3-fluorophenyl)pyrrolidine-1-carboxylate, and was isolated as a light brown solid.

Yield 1.00 g (40%). 1H NMR (400 MHz, DMSO) δ 8.63 (q, J=4.8 Hz, 1H), 8.12 (d, J=8.4 Hz, 2H), 8.00 (d, J=8.4 Hz, 2H), 4.82 (s, 2H), 2.81 (d, J=4.8 Hz, 3H). m/z: [ESI-]254, 256 (M−H)−.

### Synthesis of methyl 6-methoxy-2-(4-(methylcarbamoyl)phenyl)benzo[d]imidazo[2,1-b]thiazole-7-carboxylate (5, Scheme 73)

Compound methyl 6-methoxy-2-(4-(methylcarbamoyl)phenyl)benzo[d]imidazo[2,1-b]thiazole-7-carboxylate was prepared from methyl 2-amino-5-methoxybenzo[d]thiazole-6-carboxylate (1.00 g, 4.20 mmol) and 4-(2-bromoacetyl)-N-methylbenzamide (0.90 g, 3.51 mmol) following a similar procedure to that described for the synthesis of tert-butyl (S)-2-(4-(7-bromo-9-methyl-9H-benzo[d]imidazo[1,2-a]imidazol-2-yl)-3-fluorophenyl)pyrrolidine-1-carboxylate at 110° C., and was isolated as a light yellow solid.

Yield 0.33 g (24%). ¹H NMR (400 MHz, DMSO) δ 8.92 (s, 1H), 8.45 (q, J=4.8 Hz, 1H), 8.38 (s, 1H), 7.92 (s, 4H), 7.88 (s, 1H), 3.98 (s, 3H), 3.84 (s, 3H), 2.81 (d, J=4.8 Hz, 3H). m/z: [ESI⁺] 396 (M+H)⁺.

### Synthesis of 6-methoxy-2-(4-(methylcarbamoyl)phenyl)benzo[d]imidazo[2,1-b]thiazole-7-carboxylic Acid (6, Scheme 73)

Compound 6-methoxy-2-(4-(methylcarbamoyl)phenyl)benzo[d]imidazo[2,1-b]thiazole-7-carboxylic acid was prepared from methyl 6-methoxy-2-(4-(methylcarbamoyl)phenyl)benzo[d]imidazo[2,1-b]thiazole-7-carboxylate (0.33 g, 0.83 mmol) following a similar procedure to that described for the synthesis of 2-(1-(tert-butoxycarbonyl)-5-fluoroindolin-6-yl)benzo[d]imidazo[2,1-b]thiazole-7-carboxylic acid at room temperature, and was isolated as an off-white solid.

Yield 0.20 g (63%). ¹H NMR (400 MHz, DMSO) δ 12.77 (br s, 1H), 8.93 (s, 1H), 8.46 (q, J=4.8 Hz, 1H), 8.36 (s, 1H), 7.93 (s, 4H), 7.87 (s, 1H), 3.97 (d, 3H), 2.81 (d, J=4.8 Hz, 3H). m/z: [ESI⁺] 382 (M+H)⁺.

### Synthesis of tert-butyl 2-(3-fluoro-4-(6-methoxybenzo[d]imidazo[2,1-b]thiazol-2-yl)phenyl)pyrrolidine-1-carboxylate

Compound tert-butyl 2-(3-fluoro-4-(6-methoxybenzo[d]imidazo[2,1-b]thiazol-2-yl)phenyl)pyrrolidine-1-carboxylate was prepared from 5-methoxybenzo[d]thiazol-2-amine (0.80 g, 4.44 mmol) and tert-butyl 2-(4-(2-bromoacetyl)-3-fluorophenyl)pyrrolidine-1-carboxylate (1.89 g, 4.89 mmol) following a similar procedure to that described for the synthesis of tert-butyl (S)-2-(4-(7-bromo-9-methyl-9H-benzo[d]imidazo[1,2-a]imidazol-2-yl)-3-fluorophenyl)pyrrolidine-1-carboxylate at 110° C., and was isolated as a yellow solid.

Yield 0.90 g (43%). ¹H NMR (400 MHz, CDCl₃) δ 8.18 (dd, J=8.0, 8.0 Hz, 1H), 8.11 (s, 1H), 7.58 (d, J=8.8 Hz, 1H), 7.19 (d, J=2.4 Hz, 1H), 7.09 (dd, J=1.6, 8.0 Hz, 1H), 7.02-6.94 (m, 2H), 5.05-4.73 (m, 1H), 3.94 (s, 3H), 3.72-3.48 (m, 2H), 2.45-2.25 (m, 1H), 2.02-1.79 (m, 3H), 1.50-1.25 (m, 9H) m/z: [ESI⁺] 468 (M+H)⁺.

### Final Compounds

### Synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide Dihydrochloride (Compound 121S)

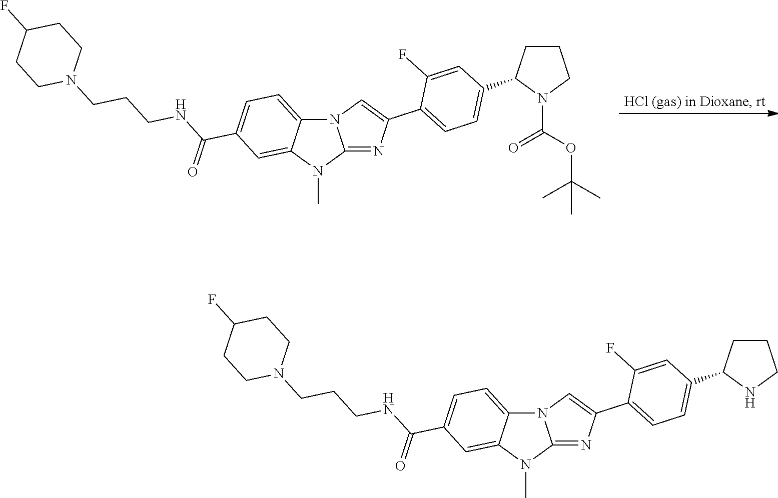 [SMILES: Cn1c2cc(C(=O)NCCCN3CCC(F)CC3)ccc2n2cc(-c3ccc([C@@H]4CCCN4C(=O)OC(C)(C)C)cc3F)nc12 ; Cn1c2cc(C(=O)NCCCN3CCC(F)CC3)ccc2n2cc(-c3ccc([C@@H]4CCCN4)cc3F)nc12]

A solution of tert-butyl (S)-2-(3-fluoro-4-(7-((3-(4-fluoropiperidin-1-yl)propyl)carbamoyl)-9-methyl-9H-benzo[d]imidazo[1,2-a]imidazol-2-yl)phenyl)pyrrolidine-1-carboxylate (200 mg, 0.322 mmol) in HCl/1,4-dioxane (4 N, 3 mL) was stirred for 2 h at room temperature under a nitrogen atmosphere. The reaction mixture was concentrated under reduced pressure. The solid was washed with dichloromethane (3×2 mL), diethyl ether (3×2 mL) and dried in a vacuum oven to afford (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride as a brown solid.

Yield 85 mg (44%). ¹H NMR (400 MHz, DMSO) δ 10.92 (br s, 1H), 10.39 (br s, 1H), 9.32 (brs, 1H), 8.97 (s, 1H), 8.55-8.46 (m, 1H), 8.40-8.29 (m, 2H), 8.14 (t, J=6.8 Hz, 1H), 7.97-7.90 (m, 1H), 7.73-7.64 (m, 1H), 7.54 (d, J=8.4 Hz, 1H), 5.12-4.71 (m, 1H), 4.66-4.55 (m, 1H), 4.10-3.97 (m, 3H), 3.57-3.47 (m, 1H), 3.45-3.39 (m, 4H), 3.35-3.24 (m, 1H), 3.22-2.97 (m, 4H), 2.47-2.37 (m, 1H), 2.35-1.96 (m, 9H). ¹⁹F NMR (376 MHz, DMSO) δ−111.83, −175.55, −186.58. m/z: [ESI⁺] 521 (M+H)⁺. (C₂₉H₃₆Cl₂F₂N₆O).

### Synthesis of (R)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide Dihydrochloride (Compound 121R)

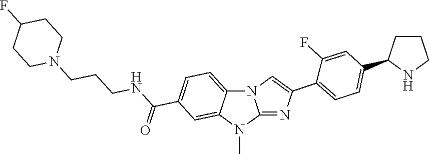 [SMILES: Cn1c2cc(C(=O)NCCCN3CCC(F)CC3)ccc2n2cc(-c3ccc([C@H]4CCCN4)cc3F)nc12]

Compound (R)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride was prepared from tert-butyl (R)-2-(3-fluoro-4-(7-((3-(4-fluoropiperidin-1-yl)propyl)carbamoyl)-9-methyl-9H-benzo[d]imidazo[1,2-a]imidazol-2-yl)phenyl)pyrrolidine-1-carboxylate (200 mg, 0.322 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride, and was isolated as a brown solid.

Yield 115 mg (60%). ¹H NMR (400 MHz, DMSO) δ 10.92 (br s, 1H), 10.39 (br s, 1H), 9.32 (brs, 1H), 8.97 (s, 1H), 8.55-8.46 (m, 1H), 8.40-8.29 (m, 2H), 8.14 (t, J=6.8 Hz, 1H), 7.97-7.90 (m, 1H), 7.73-7.64 (m, 1H), 7.54 (d, J=8.4 Hz, 1H), 5.12-4.71 (m, 1H), 4.66-4.55 (m, 1H), 4.10-3.97 (m, 3H), 3.57-3.47 (m, 1H), 3.45-3.39 (m, 4H), 3.35-3.24 (m, 1H), 3.22-2.97 (m, 4H), 2.47-2.37 (m, 1H), 2.35-1.96 (m, 9H). ¹⁹F NMR (376 MHz, DMSO) δ −112.41, −175.61, −186.59. m/z: [ESI⁺] 521 (M+H)⁺. (C₂₉H₃₆Cl₂F₂N₆O).

### Synthesis of 2-(6-fluoro-1,2,3,4-tetrahydroisoquinolin-7-yl)-N-(3-(4-fluoropiperidin-1-yl)propyl)benzo[d]imidazo[2,1-b]thiazole-7-carboxamide Dihydrochloride (Compound 301)

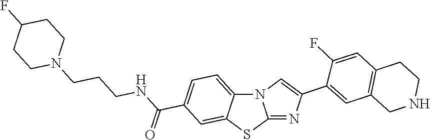 [SMILES: O=C(NCCCN1CCC(F)CC1)c1ccc2c(c1)sc1nc(-c3cc4c(cc3F)CCNC4)cn12]

Compound 2-(6-fluoro-1,2,3,4-tetrahydroisoquinolin-7-yl)-N-(3-(4-fluoropiperidin-1-yl)propyl)benzo[d]imidazo[2,1-b]thiazole-7-carboxamide dihydrochloride was prepared from tert-butyl 6-fluoro-7-(7-((3-(4-fluoropiperidin-1-yl)propyl)carbamoyl)benzo[d]imidazo[2,1-b]thiazol-2-yl)-3,4-dihydroisoquinoline-2(1H)-carboxylate (580 mg, 0.951 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride, and was isolated as a pink solid.

Yield 168 mg (30%). ¹H NMR (400 MHz, DMSO) δ 10.79 (br s, 1H), 9.60 (br s, 2H), 8.94-8.87 (m, 1H), 8.74 (d, J=3.6 Hz, 1H), 8.57 (s, 1H), 8.29 (d, J=8.4 Hz, 1H), 8.09 (dd, J=1.6, 8.4 Hz, 1H), 8.02 (d, J=7.6 Hz, 1H), 7.26 (d, J=12.0 Hz, 1H), 5.10-4.71 (m, 1H), 4.38-4.28 (m, 2H), 3.56-3.47 (m, 1H), 3.45-3.33 (m, 5H), 3.21-2.94 (m, 6H), 2.31-1.94 (m, 6H). ¹⁹F NMR (376 MHz, DMSO) δ −115.67, −175.53, −186.62. m/z: [ESI⁺] 510 (M+H)⁺. (C27H₃₁Cl₂F₂N₅OS).

### Synthesis of (S)-2-(2-fluoro-4-(pyrrolidin-2-yl)phenyl)-N-(3-(4-fluoropiperidin-1-yl)propyl)-9H-benzo[d]imidazo[1,2-a]imidazole-7-carboxamide Dihydrochloride (Compound 122S)

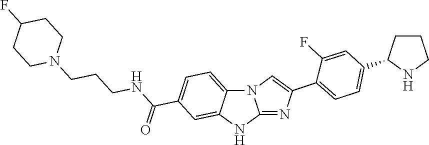 [SMILES: O=C(NCCCN1CCC(F)CC1)c1ccc2c(c1)[nH]c1nc(-c3ccc([C@@H]4CCCN4)cc3F)cn12]

Compound (S)-2-(2-fluoro-4-(pyrrolidin-2-yl)phenyl)-N-(3-(4-fluoropiperidin-1-yl)propyl)-9H-benzo[d]imidazo[1,2-a]imidazole-7-carboxamide dihydrochloride was prepared from tert-butyl (S)-2-(3-fluoro-4-(7-((3-(4-fluoropiperidin-1-yl)propyl)carbamoyl)-9H-benzo[d]imidazo[1,2-a]imidazol-2-yl)phenyl)pyrrolidine-1-carboxylate (40 mg, 0.066 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride, and was isolated as a yellow solid.

Yield 20 mg (52%). ¹H NMR (400 MHz, DMSO) δ 10.92 (br s, 1H), 10.47 (br s, 1H), 9.43 (br s, 1H), 8.96 (br s, 1H), 8.63-8.48 (m, 1H), 8.30-8.04 (m, 3H), 8.02-7.86 (m, 1H), 7.77-7.65 (m, 1H), 7.61-7.50 (m, 1H), 5.13-4.71 (m, 1H), 4.70-4.54 (m, 1H), 3.53-3.45 (m, 1H), 3.44-3.34 (m, 4H), 3.34-3.23 (m, 1H), 3.20-2.98 (m, 4H), 2.47-2.37 (m, 1H), 2.32-1.96 (m, 9H). One active proton not observed. ¹⁹F NMR (376 MHz, DMSO) δ −111.61, −175.54, −186.62. m/z: [ESI⁺] 507 (M+H)⁺. (C₂₈H₃₄Cl₂F₂N₆O).

### Synthesis of (S)-2-(2-fluoro-4-(pyrrolidin-2-yl)phenyl)-N-(3-(4-fluoropiperidin-1-yl)propyl)-3-methylbenzo[d]imidazo[2,1-b]thiazole-7-carboxamide Dihydrochloride (Compound 103S)

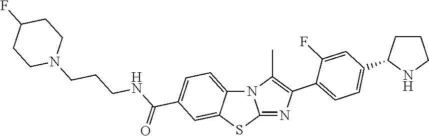 [SMILES: Cc1c(-c2ccc([C@@H]3CCCN3)cc2F)nc2sc3cc(C(=O)NCCCN4CCC(F)CC4)ccc3n12]

Compound (S)-2-(2-fluoro-4-(pyrrolidin-2-yl)phenyl)-N-(3-(4-fluoropiperidin-1-yl)propyl)-3-methylbenzo[d]imidazo[2,1-b]thiazole-7-carboxamide dihydrochloride was prepared from tert-butyl (S)-2-(3-fluoro-4-(7-((3-(4-fluoropiperidin-1-yl)propyl)carbamoyl)-3-methylbenzo[d]imidazo[2,1-b]thiazol-2-yl)phenyl)pyrrolidine-1-carboxylate (46 mg, 0.072 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride, and was isolated as a light yellow solid.

Yield 33 mg (75%). ¹H NMR (400 MHz, DMSO) δ 10.87 (br s, 1H), 10.36 (br s, 1H), 9.23 (br s, 1H), 8.93 (d, J=7.2 Hz, 1H), 8.64-8.52 (m, 1H), 8.13 (d, J=8.4 Hz, 1H), 8.07 (dd, J=2.0, 8.8 Hz, 1H), 7.74-7.67 (m, 1H), 7.67-7.58 (m, 1H), 7.53-7.48 (m, 1H), 5.10-4.72 (m, 1H), 4.70-4.58 (m, 1H), 3.59-3.47 (m, 1H), 3.46-3.35 (m, 4H), 3.35-3.24 (m, 1H), 3.22-2.96 (m, 4H), 2.72 (d, J=2.0 Hz, 3H), 2.47-2.39 (m, 1H), 2.33-1.96 (m, 9H). ¹⁹F NMR (376 MHz, DMSO) δ −113.52, −175.60, −186.61. m/z: [ESI⁺] 538 (M+H)⁺. (C₂₉H₃₅Cl₂F₂N₅OS).

### Synthesis of (R)-2-(2-fluoro-4-(pyrrolidin-2-yl)phenyl)-N-(3-(4-fluoropiperidin-1-yl)propyl)-3-methylbenzo[d]imidazo[2,1-b]thiazole-7-carboxamide Dihydrochloride (Compound 103S)

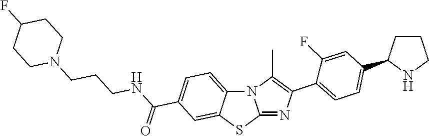 [SMILES: Cc1c(-c2ccc([C@H]3CCCN3)cc2F)nc2sc3cc(C(=O)NCCCN4CCC(F)CC4)ccc3n12]

Compound (R)-2-(2-fluoro-4-(pyrrolidin-2-yl)phenyl)-N-(3-(4-fluoropiperidin-1-yl)propyl)-3-methylbenzo[d]imidazo[2,1-b]thiazole-7-carboxamide dihydrochloride was prepared from tert-butyl (R)-2-(3-fluoro-4-(7-((3-(4-fluoropiperidin-1-yl)propyl)carbamoyl)-3-methylbenzo[d]imidazo[2,1-b]thiazol-2-yl)phenyl)pyrrolidine-1-carboxylate (38 mg, 0.060 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride, and was isolated as a light yellow solid.

Yield 26 mg (71%). ¹H NMR (400 MHz, DMSO) δ 10.73 (br s, 1H), 10.22 (br s, 1H), 9.15 (br s, 1H), 8.93 (d, J=7.2 Hz, 1H), 8.64-8.52 (m, 1H), 8.13 (d, J=8.4 Hz, 1H), 8.07 (dd, J=2.0, 8.8 Hz, 1H), 7.74-7.67 (m, 1H), 7.67-7.58 (m, 1H), 7.53-7.48 (m, 1H), 5.10-4.72 (m, 1H), 4.70-4.58 (m, 1H), 3.59-3.47 (m, 1H), 3.46-3.35 (m, 4H), 3.35-3.24 (m, 1H), 3.22-2.96 (m, 4H), 2.72 (d, J=2.0 Hz, 3H), 2.47-2.39 (m, 1H), 2.33-1.96 (m, 9H). ¹⁹F NMR (376 MHz, DMSO) δ −113.52, −175.60, −186.61. m/z: [ESI⁺]538 (M+H)⁺. (C₂₉H₃₅Cl₂F₂N₅OS).

### Synthesis of N-(3-(4-fluoropiperidin-1-yl)propyl)-6-(2-methoxyethoxy)-2-(4-(pyrrolidin-2-yl)phenyl)benzo[d]imidazo[2,1-b]thiazole-7-carboxamide Dihydrochloride (Compound 150)

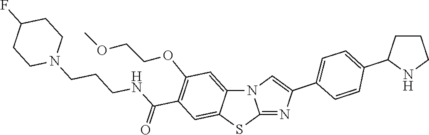 [SMILES: COCCOc1cc2c(cc1C(=O)NCCCN1CCC(F)CC1)sc1nc(-c3ccc(C4CCCN4)cc3)cn12]

Compound N-(3-(4-fluoropiperidin-1-yl)propyl)-6-(2-methoxyethoxy)-2-(4-(pyrrolidin-2-yl)phenyl)benzo[d]imidazo[2,1-b]thiazole-7-carboxamide dihydrochloride was prepared from tert-butyl 2-(4-(7-((3-(4-fluoropiperidin-1-yl)propyl)carbamoyl)-6-(2-methoxyethoxy)benzo[d]imidazo[2,1-b]thiazol-2-yl)phenyl)pyrrolidine-1-carboxylate (33 mg, 0.049 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride, and was isolated as a white solid.

Yield 13 mg (41%). ¹H NMR (400 MHz, DMSO) δ 11.06 (br s, 1H), 10.31 (br s, 1H), 9.08 (br s, 1H), 8.97-8.86 (m, 1H), 8.49-8.35 (m, 2H), 8.06-7.96 (m, 1H), 7.91 (d, J=8.0 Hz, 2H), 7.64 (d, J=8.0 Hz, 2H), 5.11-4.71 (m, 1H), 4.60-4.50 (m, 1H), 4.47-4.36 (m, 2H), 3.89-3.81 (m, 2H), 3.55-3.47 (m, 1H), 3.45-3.34 (m, 7H), 3.34-3.24 (m, 1H), 3.18-2.95 (m, 4H), 2.44-2.35 (m, 1H), 2.32-1.96 (m, 9H). ¹⁹F NMR (376 MHz, DMSO) δ −175.55, −186.68. m/z: [ESI⁺] 580 (M+H)⁺. (C₃₁H₄₀Cl₂FN₅O₃S)

### Synthesis of 6-(difluoromethoxy)-N-(3-(4-fluoropiperidin-1-yl)propyl)-2-(4-(pyrrolidin-2-yl)phenyl)benzo[d]imidazo[2,1-b]thiazole-7-carboxamide Dihydrochloride (Compound 164)

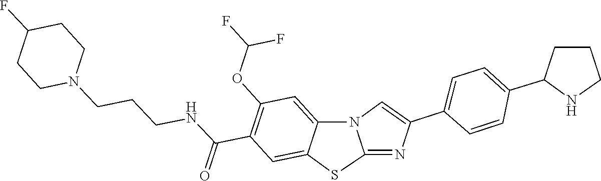 [SMILES: O=C(NCCCN1CCC(F)CC1)c1cc2sc3nc(-c4ccc(C5CCCN5)cc4)cn3c2cc1OC(F)F]

Compound 6-(difluoromethoxy)-N-(3-(4-fluoropiperidin-1-yl)propyl)-2-(4-(pyrrolidin-2-yl)phenyl)benzo[d]imidazo[2,1-b]thiazole-7-carboxamide was prepared from tert-butyl 2-(4-(6-(difluoromethoxy)-7-((3-(4-fluoropiperidin-1-yl)propyl)carbamoyl)benzo[d]imidazo[2,1-b]thiazol-2-yl)phenyl)pyrrolidine-1-carboxylate (70 mg, 0.104 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride, and was isolated as a yellow solid.

Yield 27 mg (40%). ¹H NMR (400 MHz, DMSO) δ 11.05 (br s, 1H), 10.36 (br s, 1H), 9.11 (br s, 1H), 8.98 (s, 1H), 8.74-8.62 (m, 1H), 8.32 (s, 1H), 8.05 (s, 1H), 7.92 (d, J=8.0 Hz, 2H), 7.64 (d, J=8.0 Hz, 2H), 7.33 (t, J=73.6 Hz, 1H), 5.13-4.72 (m, 1H), 4.64-4.46 (m, 1H), 3.55-3.21 (m, 6H), 3.21-2.95 (m, 4H), 2.45-1.92 (m, 10H). ¹⁹F NMR (376 MHz, DMSO) δ −81.40, −175.83, −186.73. m/z: [ESI⁺] 572 (M+H)⁺. (C₂₉H₃₄Cl₂F₃N₅O₂S)

### Synthesis of 2-(2-fluoro-4-(pyrrolidin-2-yl)phenyl)-N-(3-(4-fluoropiperidin-1-yl)propyl)-6-methoxybenzo[d]imidazo[2,1-b]thiazole-7-carboxamide Dihydrochloride (Compound 144)

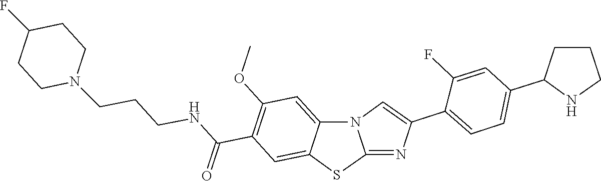 [SMILES: COc1cc2c(cc1C(=O)NCCCN1CCC(F)CC1)sc1nc(-c3ccc(C4CCCN4)cc3F)cn12]

Compound 2-(2-fluoro-4-(pyrrolidin-2-yl)phenyl)-N-(3-(4-fluoropiperidin-1-yl)propyl)-6-methoxybenzo[d]imidazo[2,1-b]thiazole-7-carboxamide dihydrochloride was prepared from tert-butyl 2-(3-fluoro-4-(7-((3-(4-fluoropiperidin-1-yl)propyl)carbamoyl)-6-methoxybenzo[d]imidazo[2,1-b]thiazol-2-yl)phenyl)pyrrolidine-1-carboxylate (60 mg, 0.092 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride, and was isolated as a yellow solid.

Yield 16 mg (28%). ¹H NMR (400 MHz, DMSO) δ 10.68 (br s, 1H), 10.14 (br s, 1H), 9.09 (br s, 1H), 8.85 (d, J=3.6 Hz, 1H), 8.50 (t, J=6.0 Hz, 1H), 8.37 (s, 1H), 8.21 (dd, J=8.0, 8.0 Hz, 1H), 8.12 (s, 1H), 7.61 (d, J=12.4 Hz, 1H), 7.47 (dd, J=1.6, 8.0 Hz, 1H), 5.13-4.71 (m, 1H), 4.65-4.52 (m, 1H), 4.04 (s, 3H), 3.45-3.23 (m, 6H), 3.16-2.94 (m, 4H), 2.46-2.33 (m, 1H), 2.26-1.92 (m, 9H). ¹⁹F NMR (376 MHz, DMSO) δ −113.03, −175.58, −186.61. m/z: [ESI⁺] 554 (M+H)⁺. (C₂₉H₃₅Cl₂F₂N₅O₂S)

### Synthesis of (R)-2-(2-fluoro-4-(pyrrolidin-2-yl)phenyl)-N-(3-(4-fluoropiperidin-1-yl)propyl)-6-methoxybenzo[d]imidazo[2,1-b]thiazole-7-carboxamide Dihydrochloride (Compound 149R)

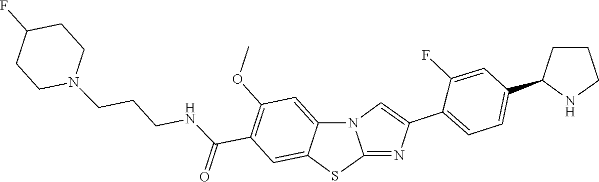 [SMILES: COc1cc2c(cc1C(=O)NCCCN1CCC(F)CC1)sc1nc(-c3ccc([C@H]4CCCN4)cc3F)cn12]

Compound (R)-2-(2-fluoro-4-(pyrrolidin-2-yl)phenyl)-N-(3-(4-fluoropiperidin-1-yl)propyl)-6-methoxybenzo[d]imidazo[2,1-b]thiazole-7-carboxamide dihydrochloride was prepared from tert-butyl (R)-2-(3-fluoro-4-(7-((3-(4-fluoropiperidin-1-yl)propyl)carbamoyl)-6-methoxybenzo[d]imidazo[2,1-b]thiazol-2-yl)phenyl)pyrrolidine-1-carboxylate (35 mg, 0.054 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride, and was isolated as a yellow solid.

Yield 10 mg (30%). ¹H NMR (400 MHz, DMSO) δ 10.85 (br s, 1H), 10.24 (br s, 1H), 9.14 (br s, 1H), 8.85 (d, J=3.6 Hz, 1H), 8.50 (t, J=6.0 Hz, 1H), 8.37 (s, 1H), 8.21 (dd, J=8.0, 8.0 Hz, 1H), 8.12 (s, 1H), 7.61 (d, J=12.4 Hz, 1H), 7.47 (dd, J=1.6, 8.0 Hz, 1H), 5.13-4.71 (m, 1H), 4.65-4.52 (m, 1H), 4.04 (s, 3H), 3.45-3.23 (m, 6H), 3.16-2.94 (m, 4H), 2.46-2.33 (m, 1H), 2.26-1.92 (m, 9H). ¹⁹F NMR (376 MHz, DMSO) δ −113.03. Aliphatic ¹⁹F signal not observed. m/z: [ESI⁺] 554 (M+H)⁺. (C₂₉H₃₅Cl₂F₂N₅O₂S)

### Synthesis of (S)-2-(2-fluoro-4-(pyrrolidin-2-yl)phenyl)-N-(3-(4-fluoropiperidin-1-yl)propyl)-6-methoxy benzo[d]imidazo[2,1-b]thiazole-7-carboxamide Dihydrochloride (Compound 149S)

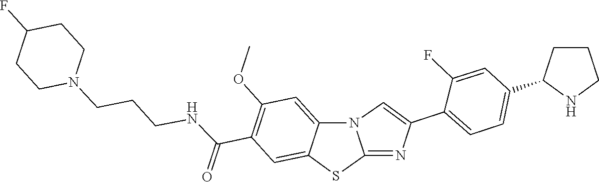 [SMILES: COc1cc2c(cc1C(=O)NCCCN1CCC(F)CC1)sc1nc(-c3ccc([C@@H]4CCCN4)cc3F)cn12]

Compound (S)-2-(2-fluoro-4-(pyrrolidin-2-yl)phenyl)-N-(3-(4-fluoropiperidin-1-yl)propyl)-6-methoxybenzo[d]imidazo[2,1-b]thiazole-7-carboxamide dihydrochloride was prepared from tert-butyl (S)-2-(3-fluoro-4-(7-((3-(4-fluoropiperidin-1-yl)propyl)carbamoyl)-6-methoxybenzo[d]imidazo[2,1-b]thiazol-2-yl)phenyl)pyrrolidine-1-carboxylate (35 mg, 0.054 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride, and was isolated as a yellow solid.

Yield 12 mg (36%). ¹H NMR (400 MHz, DMSO) δ 10.85 (br s, 1H), 10.24 (br s, 1H), 9.14 (br s, 1H), 8.85 (d, J=3.6 Hz, 1H), 8.50 (t, J=6.0 Hz, 1H), 8.37 (s, 1H), 8.21 (dd, J=8.0, 8.0 Hz, 1H), 8.12 (s, 1H), 7.61 (d, J=12.4 Hz, 1H), 7.47 (dd, J=1.6, 8.0 Hz, 1H), 5.13-4.71 (m, 1H), 4.65-4.52 (m, 1H), 4.04 (s, 3H), 3.45-3.23 (m, 6H), 3.16-2.94 (m, 4H), 2.46-2.33 (m, 1H), 2.26-1.92 (m, 9H). ¹⁹F NMR (376 MHz, DMSO) δ −113.03. Aliphatic ¹⁹F signal not observed. m/z: [ESI⁺] 554 (M+H)⁺. (C₂₉H₃₅Cl₂F₂N₅O₂S)

### Synthesis of 2-(2-fluoro-4-(pyrrolidin-2-yl)phenyl)-6-methoxybenzo[d]imidazo[2,1-b]thiazole-7-carboxamide Hydrochloride (Compound 155)

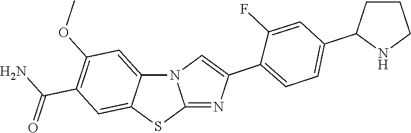 [SMILES: COc1cc2c(cc1C(N)=O)sc1nc(-c3ccc(C4CCCN4)cc3F)cn12]

Compound 2-(2-fluoro-4-(pyrrolidin-2-yl)phenyl)-6-methoxybenzo[d]imidazo[2,1-b]thiazole-7-carboxamide hydrochloride was prepared from tert-butyl 2-(4-(7-carbamoyl-6-methoxybenzo[d]imidazo[2,1-b]thiazol-2-yl)-3-fluorophenyl)pyrrolidine-1-carboxylate (50 mg, 0.098 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride, and was isolated as a white solid.

Yield 20 mg (46%). ¹H NMR (400 MHz, DMSO) δ 10.19 (br s, 1H), 9.11 (br s, 1H), 8.85 (d, J=3.6 Hz, 1H), 8.43 (s, 1H), 8.21 (dd, J=8.0, 8.0 Hz, 1H), 8.12 (s, 1H), 7.75 (br s, 1H), 7.67 (br s, 1H), 7.62 (dd, J=1.6, 12.4 Hz, 1H), 7.47 (dd, J=1.6, 8.0 Hz, 1H), 4.66-4.55 (m, 1H), 4.04 (s, 3H), 3.45-3.23 (m, 2H), 2.47-2.36 (m, 1H), 2.19-1.98 (m, 3H). ¹⁹F NMR (376 MHz, DMSO) δ −112.99. m/z: [ESI⁺] 411 (M+H)⁺. (C₂₁H₂₀ClFN₄O₂S)

### Synthesis of 2-(2-fluoro-4-(pyrrolidin-2-yl)phenyl)-6-methoxy-N-methylbenzo[d]imidazo[2,1-b]thiazol e-7-carboxamide Hydrochloride (Compound 160)

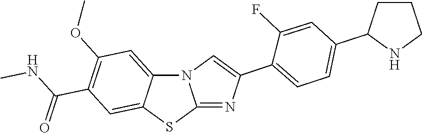 [SMILES: CNC(=O)c1cc2sc3nc(-c4ccc(C5CCCN5)cc4F)cn3c2cc1OC]

Compound 2-(2-fluoro-4-(pyrrolidin-2-yl)phenyl)-6-methoxy-N-methylbenzo[d]imidazo[2,1-b]thiazole-7-carboxamide hydrochloride was prepared from tert-butyl 2-(3-fluoro-4-(6-methoxy-7-(methylcarbamoyl)benzo[d]imidazo[2,1-b]thiazol-2-yl)phenyl)pyrrolidine-1-carboxylate (20 mg, 0.038 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride, and was isolated as a light yellow solid.

Yield 10 mg (57%). ¹H NMR (400 MHz, DMSO) δ 10.39 (br s, 1H), 9.23 (br s, 1H), 8.85 (d, J=3.6 Hz, 1H), 8.38 (s, 1H), 8.33-8.24 (m, 1H), 8.24-8.15 (m, 1H), 8.12 (s, 1H), 7.64 (d, J=12.4 Hz, 1H), 7.50-4.46 (m, 1H), 4.64-4.52 (m, 1H), 4.03 (s, 3H), 3.47-3.21 (m, 2H), 2.84 (d, J=4.4 Hz, 3H), 2.46-2.34 (m, 1H), 2.18-1.96 (m, 3H). ¹⁹F NMR (376 MHz, DMSO) δ −112.96. m/z: [ESI⁺] 425 (M+H)⁺. (C₂₂H₂₂ClFN₄O₂S)

### Synthesis of 2-(2-fluoro-4-(pyrrolidin-2-yl)phenyl)-6-methoxy-N-(1-methylpiperidin-4-yl)benzo[d]imidazo[2,1-b]thiazole-7-carboxamide Dihydrochloride (Compound 175)

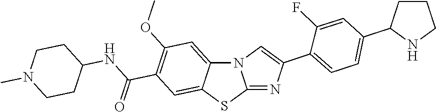 [SMILES: COc1cc2c(cc1C(=O)NC1CCN(C)CC1)sc1nc(-c3ccc(C4CCCN4)cc3F)cn12]

Compound 2-(2-fluoro-4-(pyrrolidin-2-yl)phenyl)-6-methoxy-N-(1-methylpiperidin-4-yl)benzo[d]imidazo[2,1-b]thiazole-7-carboxamide dihydrochloride was prepared from tert-butyl 2-(3-fluoro-4-(6-methoxy-7-((1-methylpiperidin-4-yl)carbamoyl)benzo[d]imidazo[2,1-b]thiazol-2-yl)phenyl)pyrrolidine-1-carboxylate (100 mg, 0.165 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride, and was isolated as an off-white solid.

Yield 30 mg (31%). ¹H NMR (400 MHz, DMSO) δ 10.65 (br s, 1H), 10.26 (br s, 1H), 9.15 (br s, 1H), 8.84 (d, J=3.6 Hz, 1H), 8.36-8.28 (m, 1H), 8.24-8.18 (m, 2H), 8.14-8.09 (m, 1H), 7.63 (dd, J=1.6, 12.4 Hz, 1H), 7.48 (dd, J=1.6, 8.0 Hz, 1H), 4.65-4.57 (m, 1H), 4.25-3.93 (m, 4H), 3.49-3.23 (m, 4H), 3.23-3.02 (m, 2H), 2.80-2.67 (m, 3H), 2.45-2.36 (m, 1H), 2.18-1.96 (m, 5H), 1.94-1.81 (m, 2H). ¹⁹F NMR (376 MHz, DMSO) δ −113.05. m/z: [ESI⁺] 508 (M+H)⁺. (C₂₇H₃₂Cl₂FN₅O₂S)

### Synthesis of 2-(2-fluoro-4-(pyrrolidin-2-yl)phenyl)-6-methoxy-N—((R)-1-methylpiperidin-3-yl)benzo[d]imidazo[2,1-b]thiazole-7-carboxamide Dihydrochloride (Compound 153R)

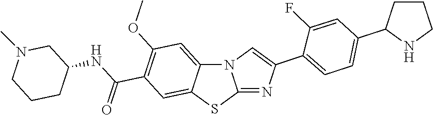 [SMILES: COc1cc2c(cc1C(=O)N[C@@H]1CCCN(C)C1)sc1nc(-c3ccc(C4CCCN4)cc3F)cn12]

Compound 2-(2-fluoro-4-(pyrrolidin-2-yl)phenyl)-6-methoxy-N—((R)-1-methylpiperidin-3-yl)benzo[d]imidazo[2,1-b]thiazole-7-carboxamide dihydrochloride was prepared from tert-butyl 2-(3-fluoro-4-(6-methoxy-7-(((R)-1-methylpiperidin-3-yl)carbamoyl)benzo[d]imidazo[2,1-b]thiazol-2-yl)phenyl)pyrrolidine-1-carboxylate (50 mg, 0.082 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride, and was isolated as a white solid.

Yield 20 mg (42%). ¹H NMR (400 MHz, DMSO) δ 10.95 (br s, 0.24H), 10.81 (br s, 0.76H), 10.35 (br s, 1H), 9.21 (br s, 1H), 8.85 (dd, J=1.2, 3.6 Hz, 0.76H), 8.73 (d, J=7.6 Hz, 0.24H), 8.35-8.31 (m, 1H), 8.27 (s, 1H), 8.21 (dd, J=1.6, 8.0 Hz, 1H), 8.12 (d, J=8.4 Hz, 1H), 7.64 (dd, J=1.6, 12.4 Hz, 1H), 7.48 (dd, J=1.6, 8.4 Hz, 1H), 4.67-4.54 (m, 1H), 4.48-4.37 (m, 0.24H), 4.37-4.19 (m, 0.76H), 4.01 (s, 3H), 3.57-3.45 (m, 1H), 3.45-3.19 (m, 4H), 3.04-2.81 (m, 2H), 2.79 (d, J=4.8 Hz, 2.28H, part of NCH₃), 2.75 (d, J=4.8 Hz, 0.72H, part of NCH₃), 2.45-2.35 (m, 1H), 2.21-1.72 (m, 5H), 1.60-1.44 (m, 1H). ¹⁹F NMR (376 MHz, DMSO) δ −113.03. m/z: [ESI⁺] 508 (M+H)⁺. (C₂₇H₃₂Cl₂FN₅O₂S). Isolated as 19:6 ratio of two diastereomers.

### Synthesis of 2-(2-fluoro-4-(pyrrolidin-2-yl)phenyl)-6-methoxy-N—((S)-1-methylpiperidin-3-yl)benzo[d]imidazo[2,1-b]thiazole-7-carboxamide Dihydrochloride (Compound 153S)

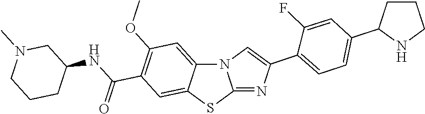 [SMILES: COc1cc2c(cc1C(=O)N[C@H]1CCCN(C)C1)sc1nc(-c3ccc(C4CCCN4)cc3F)cn12]

Compound 2-(2-fluoro-4-(pyrrolidin-2-yl)phenyl)-6-methoxy-N—((S)-1-methylpiperidin-3-yl)benzo[d]imidazo[2,1-b]thiazole-7-carboxamide dihydrochloride was prepared from tert-butyl 2-(3-fluoro-4-(6-methoxy-7-(((S)-1-methylpiperidin-3-yl)carbamoyl)benzo[d]imidazo[2,1-b]thiazol-2-yl)phenyl)pyrrolidine-1-carboxylate (50 mg, 0.082 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride, and was isolated as a light yellow solid.

Yield 20 mg (42%). 1H NMR (400 MHz, DMSO) δ 10.95 (br s, 0.24H), 10.81 (br s, 0.76H), 10.35 (br s, 1H), 9.21 (br s, 1H), 8.85 (dd, J=1.2, 3.6 Hz, 0.76H), 8.73 (d, J=7.6 Hz, 0.24H), 8.35-8.31 (m, 1H), 8.27 (s, 1H), 8.21 (dd, J=1.6, 8.0 Hz, 1H), 8.12 (d, J=8.4 Hz, 1H), 7.64 (dd, J=1.6, 12.4 Hz, 1H), 7.48 (dd, J=1.6, 8.4 Hz, 1H), 4.67-4.54 (m, 1H), 4.48-4.37 (m, 0.24H), 4.37-4.19 (m, 0.76H), 4.01 (s, 3H), 3.57-3.45 (m, 1H), 3.45-3.19 (m, 4H), 3.04-2.81 (m, 2H), 2.79 (d, J=4.8 Hz, 2.28H, part of NCH3), 2.75 (d, J=4.8 Hz, 0.72H, part of NCH3), 2.45-2.35 (m, 1H), 2.21-1.72 (m, 5H), 1.60-1.44 (m, 1H). 19F NMR (376 MHz, DMSO) δ −113.03. m/z: [ESI⁺] 508 (M+H)⁺. (C27H32C12FN5O2S). Isolated as 19:6 ratio of two diastereomers.

### Synthesis of 2-(2-fluoro-4-(pyrrolidin-2-yl)phenyl)-N-(3-(4-fluoropiperidin-1-yl)propyl)-6-(2-methoxy ethoxy)benzo[d]imidazo[2,1-b]thiazole-7-carboxamide Dihydrochloride (Compound 166)

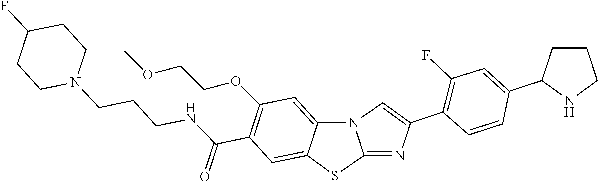 [SMILES: COCCOc1cc2c(cc1C(=O)NCCCN1CCC(F)CC1)sc1nc(-c3ccc(C4CCCN4)cc3F)cn12]

Compound 2-(2-fluoro-4-(pyrrolidin-2-yl)phenyl)-N-(3-(4-fluoropiperidin-1-yl)propyl)-6-(2-methoxyethoxy)benzo[d]imidazo[2,1-b]thiazole-7-carboxamide dihydrochloride was prepared from tert-butyl 2-(3-fluoro-4-(7-((3-(4-fluoropiperidin-1-yl)propyl)carbamoyl)-6-(2-methoxyethoxy)benzo[d]imidazo[2,1-b]thiazol-2-yl)phenyl)pyrrolidine-1-carboxylate (105 mg, 0.150 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride, and was isolated as an off-white solid.

Yield 96 mg (95%). ¹H NMR (400 MHz, DMSO) δ 11.02 (br s, 1H), 10.35 (br s, 1H), 9.22 (br s, 1H), 8.80 (d, J=3.6 Hz, 1H), 8.42 (s, 1H), 8.25-8.14 (m, 2H), 7.64 (dd, J=1.6, 12.4 Hz, 1H), 7.48 (dd, J=1.6, 8.0 Hz, 1H), 5.10-4.70 (m, 1H), 4.67-4.51 (m, 1H), 4.46-4.35 (m, 2H), 3.90-3.81 (m, 2H), 3.55-3.46 (m, 1H), 3.45-3.35 (m, 8H), 3.33-3.21 (m, 1H), 3.20-2.93 (m, 4H), 2.44-2.34 (m, 1H), 2.34-1.95 (m, 9H). ¹⁹F NMR (376 MHz, DMSO) δ −113.05, −175.57, −186.72. m/z: [ESI⁺] 598 (M+H)⁺. (C₃₁H₃₉Cl₂F₂N₅O₃S)

### Synthesis of 2-(6-fluoroindolin-5-yl)-N-(3-(4-fluoropiperidin-1-yl)propyl)benzo[d]imidazo[2,1-b]thiazole-7-carboxamide (Compound 303)

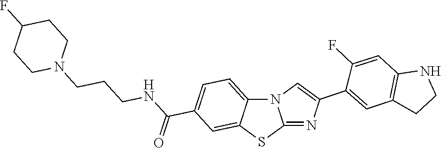 [SMILES: O=C(NCCCN1CCC(F)CC1)c1ccc2c(c1)sc1nc(-c3cc4c(cc3F)NCC4)cn12]

A mixture of tert-butyl 6-fluoro-5-(7-((3-(4-fluoropiperidin-1-yl)propyl)carbamoyl)benzo[d]imidazo[2,1-b]thiazol-2-yl)indoline-1-carboxylate (80 mg, 0.134 mmol) in a solution of hydrochloride (gas) in 1,4-dioxane (4 N, 2 mL) and dichloromethane (2 mL) was stirred for 3 h at room temperature under a nitrogen atmosphere. The resulting mixture was concentrated under reduced pressure. The residue was basified to pH 7 with an aqueous solution of saturated sodium bicarbonate. The resulting mixture was concentrated under reduced pressure. The residue was purified by reverse phase flash column chromatography with the following conditions; column, C18 silica gel, 20-40 um, 120 g; Mobile phase A: water (10 mmol/L ammonium bicarbonate); Mobile phase B: acetonitrile; Gradient: 30%-50% B in 25 min; Flow rate: 60 mL/min; Detector, UV 254 nm. The fractions containing desired product were collected at 45% B and concentrated under reduced pressure to afford 2-(6-fluoroindolin-5-yl)-N-(3-(4-fluoropiperidin-1-yl)propyl)benzo[d]imidazo[2,1-b]thiazole-7-carboxamide as a white solid.

Yield 8 mg (12%). ¹H NMR (400 MHz, DMSO) δ 8.58 (t, J=5.6 Hz, 1H), 8.46 (d, J=1.6 Hz, 1H), 8.38 (d, J=3.6 Hz, 1H), 8.18 (d, J=8.4 Hz, 1H), 7.99 (dd, J=1.6, 8.4 Hz, 1H), 7.74 (d, J=7.6 Hz, 1H), 6.35 (d, J=12.4 Hz, 1H), 5.95 (s, 1H), 4.76-4.57 (m, 1H), 3.56-3.49 (m, 2H), 3.33-3.32 (m, 2H), 2.96 (t, J=8.4 Hz, 2H), 2.59-2.51 (m, 2H), 2.36 (t, J=7.2 Hz, 2H), 2.32-2.24 (m, 2H), 1.93-1.77 (m, 2H), 1.76-1.63 (m, 4H). ¹⁹F NMR (376 MHz, DMSO) δ −115.61, Aliphatic ¹⁹F signal not observed. m/z: [ESI⁺] 496 (M+H)⁺. (C₂₆H₂₇F₂N₅OS).

### Synthesis of (S)-2-(2-fluoro-4-(pyrrolidin-2-yl)phenyl)-N-(3-(4-fluoropiperidin-1-yl)propyl)-6-methylbenzo[d]imidazo[2,1-b]thiazole-7-carboxamide (Compound 102S)

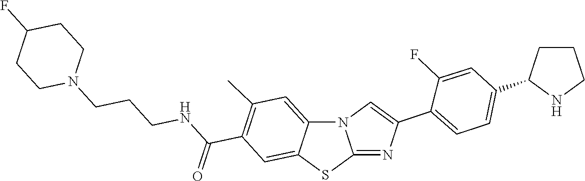 [SMILES: Cc1cc2c(cc1C(=O)NCCCN1CCC(F)CC1)sc1nc(-c3ccc([C@@H]4CCCN4)cc3F)cn12]

Compound (S)-2-(2-fluoro-4-(pyrrolidin-2-yl)phenyl)-N-(3-(4-fluoropiperidin-1-yl)propyl)-6-methylbenzo[d]imidazo[2,1-b]thiazole-7-carboxamide was prepared from tert-butyl (S)-2-(3-fluoro-4-(7-((3-(4-fluoropiperidin-1-yl)propyl)carbamoyl)-6-methylbenzo[d]imidazo[2,1-b]thiazol-2-yl)phenyl)pyrrolidine-1-carboxylate (150 mg, 0.235 mmol) following a similar procedure to that described for the synthesis of 2-(6-fluoroindolin-5-yl)-N-(3-(4-fluoropiperidin-1-yl)propyl)benzo[d]imidazo[2,1-b]thiazole-7-carboxamide, and was isolated as a light yellow solid.

Yield 50 mg (40%). ¹H NMR (400 MHz, DMSO) δ 8.59 (d, J=3.6 Hz, 1H), 8.41 (t, J=5.6 Hz, 1H), 8.10 (d, J=4.4 Hz, 1H), 8.05 (t, J=8.0 Hz, 1H), 7.99 (s, 1H), 7.37-7.06 (m, 2H), 4.91-4.56 (m, 1H), 4.10 (t, J=7.6 Hz, 1H), 3.29-3.23 (m, 2H), 3.05-2.97 (m, 1H), 2.95-2.86 (m, 1H), 2.60-2.52 (m, 2H), 2.48 (s, 3H), 2.37 (t, J=7.2 Hz, 2H), 2.34-2.24 (m, 2H), 2.22-2.10 (m, 1H), 1.93-1.61 (m, 8H), 1.56-1.45 (m, 1H). Active NH proton of pyrrolidine not observed. ¹⁹F NMR (376 MHz, DMSO) δ −114.20. Aliphatic ¹⁹F signal not observed. m/z: [ESI⁺] 538 (M+H)⁺. (C₂₉H₃₃F₂N₅OS).

### Synthesis of (R)-2-(2-fluoro-4-(pyrrolidin-2-yl)phenyl)-N-(3-(4-fluoropiperidin-1-yl)propyl)-6-methylbenzo[d]imidazo[2,1-b]thiazole-7-carboxamide (Compound 102R)

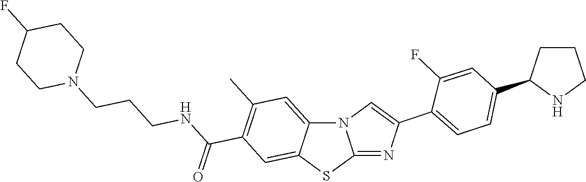 [SMILES: Cc1cc2c(cc1C(=O)NCCCN1CCC(F)CC1)sc1nc(-c3ccc([C@H]4CCCN4)cc3F)cn12]

Compound (R)-2-(2-fluoro-4-(pyrrolidin-2-yl)phenyl)-N-(3-(4-fluoropiperidin-1-yl)propyl)-6-methylbenzo[d]imidazo[2,1-b]thiazole-7-carboxamide was prepared from tert-butyl (R)-2-(3-fluoro-4-(7-((3-(4-fluoropiperidin-1-yl)propyl)carbamoyl)-6-methylbenzo[d]imidazo[2,1-b]thiazol-2-yl)phenyl)pyrrolidine-1-carboxylate (110 mg, 0.172 mmol) following a similar procedure to that described for the synthesis of 2-(6-fluoroindolin-5-yl)-N-(3-(4-fluoropiperidin-1-yl)propyl)benzo[d]imidazo[2,1-b]thiazole-7-carboxamide, and was isolated as a white solid.

Yield 40 mg (43%). ¹H NMR (400 MHz, DMSO) δ 8.59 (d, J=3.6 Hz, 1H), 8.41 (t, J=5.6 Hz, 1H), 8.10 (d, J=4.4 Hz, 1H), 8.05 (t, J=8.0 Hz, 1H), 7.99 (s, 1H), 7.37-7.06 (m, 2H), 4.91-4.56 (m, 1H), 4.10 (t, J=7.6 Hz, 1H), 3.29-3.23 (m, 2H), 3.05-2.97 (m, 1H), 2.95-2.86 (m, 1H), 2.60-2.52 (m, 2H), 2.48 (s, 3H), 2.37 (t, J=7.2 Hz, 2H), 2.34-2.24 (m, 2H), 2.22-2.10 (m, 1H), 1.93-1.61 (m, 8H), 1.56-1.45 (m, 1H). Pyrrolidine N—H not observed. ¹⁹F NMR (376 MHz, DMSO) δ −114.20. Aliphatic ¹⁹F signal not observed. m/z: [ESI⁺] 538 (M+H)⁺. (C₂₉H₃₃F₂N₅OS).

### Synthesis of N-(3-(4-fluoropiperidin-1-yl)propyl)-6-methoxy-2-(pyridin-4-yl)benzo[d]imidazo[2,1-b]thiazole-7-carboxamide (Compound 161)

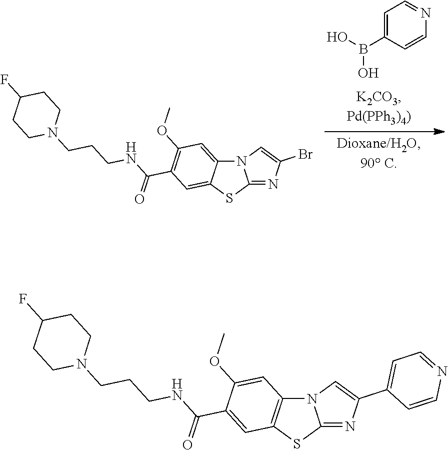 [SMILES: OB(O)c1ccncc1 ; COc1cc2c(cc1C(=O)NCCCN1CCC(F)CC1)sc1nc(Br)cn12 ; COc1cc2c(cc1C(=O)NCCCN1CCC(F)CC1)sc1nc(-c3ccncc3)cn12]

To a stirred mixture of 2-bromo-N-(3-(4-fluoropiperidin-1-yl)propyl)-6-methoxybenzo[d]imidazo[2,1-b]thiazole-7-carboxamide (60 mg, 0.128 mmol) and pyridin-4-ylboronic acid (23 mg, 0.187 mmol) in 1,4-dioxane (1 mL) and water (0.2 mL) were added potassium carbonate (34 mg, 0.246 mmol) and tetrakis(triphenylphosphine)palladium (14 mg, 0.012 mmol) at room temperature under a nitrogen atmosphere. The resulting mixture was stirred for 16 h at 90° C. The mixture was cooled to room temperature and purified directly by reverse phase flash column chromatography with the following conditions; Column: WelFlash™ C18-I, 20-40 μm, 80 g; Eluent A: water (plus 10 mmol/L ammonium bicarbonate); Eluent B: acetonitrile; Gradient: 45%-60% B in 25 min; Flow rate: 30 mL/min; Detector: UV 220/254 nm. Desired fractions were collected at 58% B, concentrated under reduced pressure and lyophilized to afford N-(3-(4-fluoropiperidin-1-yl)propyl)-6-methoxy-2-(pyridin-4-yl)benzo[d]imidazo[2,1-b]thiazole-7-carboxamide as a white solid.

Yield 8 mg (13%). ¹H NMR (400 MHz, DMSO) δ 9.06 (s, 1H), 8.63 (d, J=6.0 Hz, 2H), 8.34 (s, 1H), 8.28 (t, J=5.6 Hz, 1H), 7.89 (s, 1H), 7.78 (d, J=6.0 Hz, 2H), 4.79-4.53 (m, 1H), 4.02 (s, 3H), 3.37-3.29 (m, 2H), 2.57-2.50 (m, 2H), 2.40-2.32 (m, 2H), 2.32-2.24 (m, 2H), 1.94-1.77 (m, 2H), 1.77-1.64 (m, 4H). ¹⁹F signal not observed. m/z: [ESI⁺] 468 (M+H)⁺. (C₂₄H₂₆FN₅O₂S)

### Synthesis of 2-(2-fluoro-4-(methylcarbamoyl)phenyl)-N-(3-(4-fluoropiperidin-1-yl)propyl)-6-methoxybenzo[d]imidazo[2,1-b]thiazole-7-carboxamide Hydrochloride (Compound 159)

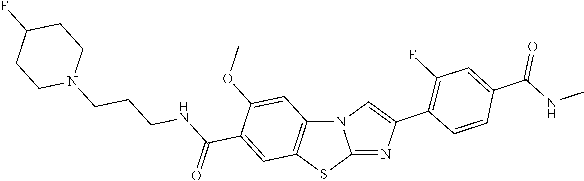 [SMILES: CNC(=O)c1ccc(-c2cn3c(n2)sc2cc(C(=O)NCCCN4CCC(F)CC4)c(OC)cc23)c(F)c1]

Compound 2-(2-fluoro-4-(methylcarbamoyl)phenyl)-N-(3-(4-fluoropiperidin-1-yl)propyl)-6-methoxybenzo[d]imidazo[2,1-b]thiazole-7-carboxamide hydrochloride was prepared from 2-bromo-N-(3-(4-fluoropiperidin-1-yl)propyl)-6-methoxybenzo[d]imidazo[2,1-b]thiazole-7-carboxamide (100 mg, 0.213 mmol) and (2-fluoro-4-(methylcarbamoyl)phenyl)boronic acid (60 mg, 0.305 mmol) following a similar procedure to that described for the synthesis of N-(3-(4-fluoropiperidin-1-yl)propyl)-6-methoxy-2-(pyridin-4-yl)benzo[d]imidazo[2,1-b]thiazole-7-carboxamide. After completion, the resulting solution was cooled to room temperature and purified directly by reverse phase flash column chromatography with the following conditions; Column: WelFlash™ C18-I, 20-40 μm, 120 g; Eluent A: water (plus 10 mmol/L hydrochloride acid); Eluent B: acetonitrile; Gradient: 20%-40% B in 25 min; Flow rate: 45 mL/min; Detector: UV 220/254 nm. Desired fractions were collected at 29% B, concentrated under reduced pressure, and lyophilized to afford 2-(2-fluoro-4-(methylcarbamoyl)phenyl)-N-(3-(4-fluoropiperidin-1-yl)propyl)-6-methoxybenzo[d]imidazo[2,1-b]thiazole-7-carboxamide as a yellow solid.

Yield 20 mg (16%). ¹H NMR (400 MHz, DMSO) δ 10.55 (br s, 1H), 8.89 (d, J=3.6 Hz, 1H), 8.66-8.55 (m, 1H), 8.55-8.45 (m, 1H), 8.37 (s, 1H), 8.24 (t, J=8.0 Hz, 1H), 8.14 (s, 1H), 7.87-7.76 (m, 2H), 5.13-4.71 (m, 1H), 4.05 (s, 3H), 3.56-3.47 (m, 1H), 3.47-3.33 (m, 3H), 3.25-2.94 (m, 4H), 2.91-2.77 (m, 3H), 2.38-1.89 (m, 6H). ¹⁹F NMR (376 MHz, DMSO) δ −113.36, −175.87, −186.63. m/z: [ESI⁺] 542 (M+H)⁺. (C₂₇H₃₀ClF₂N₅O₃S)

### Synthesis of 2-(2-fluoro-4-(5-oxopyrrolidin-2-yl)phenyl)-N-(3-(4-fluoropiperidin-1-yl)propyl)-6-methoxybenzo[d]imidazo[2,1-b]thiazole-7-carboxamide (Compound 151)

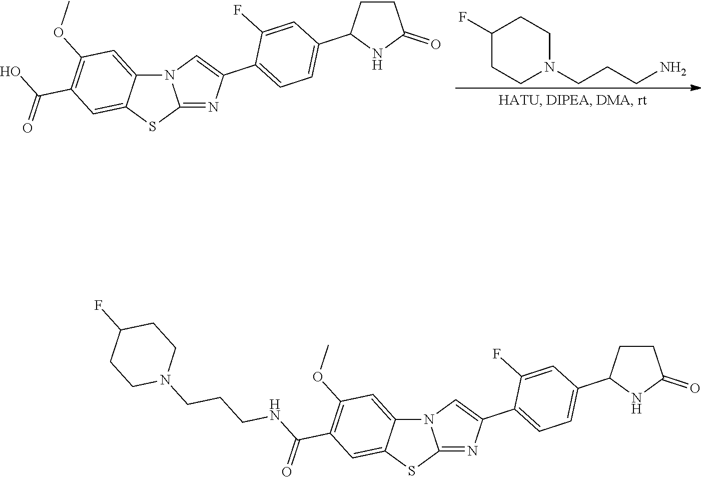

Compound 2-(2-fluoro-4-(5-oxopyrrolidin-2-yl)phenyl)-N-(3-(4-fluoropiperidin-1-yl)propyl)-6-methoxybenzo[d]imidazo[2,1-b]thiazole-7-carboxamide was prepared from 2-(2-fluoro-4-(5-oxopyrrolidin-2-yl)phenyl)-6-methoxybenzo[d]imidazo[2,1-b]thiazole-7-carboxylic acid (70 mg, 0.165 mmol) and 3-(4-fluoropiperidin-1-yl)propan-1-amine (40 mg, 0.250 mmol) following a similar procedure to that described for the synthesis of tert-butyl 5-fluoro-6-(7-((3-(4-fluoropiperidin-1-yl)propyl)carbamoyl)benzo[d]imidazo[2,1-b]thiazol-2-yl)indoline-1-carboxylate, and was purified by reverse phase flash column chromatography with the following conditions; Column: C18 Column 80 g; Mobile Phase A: water (10 mmol/L ammonium bicarbonate), Mobile Phase B: methanol; Flow rate: 30 mL/min; Gradient: 55%-70% B in 30 min; Detector: UV 254/215 nm. The fractions containing the desired product were collected at 65% B, concentrated under reduced pressure and lyophilized to afford 2-(2-fluoro-4-(5-oxopyrrolidin-2-yl)phenyl)-N-(3-(4-fluoropiperidin-1-yl)propyl)-6-methoxybenzo[d]imidazo[2,1-b]thiazole-7-carboxamide as a white solid.

Yield 17 mg (18%). ¹H NMR (400 MHz, DMSO) δ 8.77 (d, J=3.6 Hz, 1H), 8.33 (s, 1H), 8.27 (t, J=5.6 Hz, 1H), 8.18-8.12 (m, 2H), 8.09 (s, 1H), 7.33-7.22 (m, 2H), 4.78-4.69 (m, 1H), 4.78-4.56 (m, 1H), 4.02 (s, 3H), 3.40-3.26 (m, 2H), 2.60-2.47 (m, 3H), 2.40-2.21 (m, 6H), 1.93-1.76 (m, 3H), 1.76-1.61 (m, 4H). ¹⁹F NMR (376 MHz, DMSO) δ −113.51, Aliphatic ¹⁹F signal not observed. m/z: [ESI⁺] 568 (M+H)⁺. (C₂₉H₃₁F₂N₅O₃S)

### Synthesis of N-(3-(4-fluoropiperidin-1-yl)propyl)-6-methoxy-2-(4-(methylcarbamoyl)phenyl)benzo[d]imidazo[2,1-b]thiazole-7-carboxamide (Compound 157)

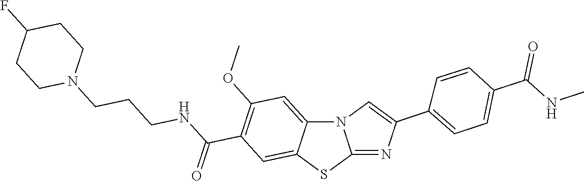 [SMILES: CNC(=O)c1ccc(-c2cn3c(n2)sc2cc(C(=O)NCCCN4CCC(F)CC4)c(OC)cc23)cc1]

Compound N-(3-(4-fluoropiperidin-1-yl)propyl)-6-methoxy-2-(4-(methylcarbamoyl)phenyl)benzo[d]imidazo[2,1-b]thiazole-7-carboxamide was prepared from 6-methoxy-2-(4-(methylcarbamoyl)phenyl)benzo[d]imidazo[2,1-b]thiazole-7-carboxylic acid (150 mg, 0.393 mmol) and 3-(4-fluoropiperidin-1-yl)propan-1-amine (82 mg, 0.512 mmol) following a similar procedure to that described for the synthesis of 2-(2-fluoro-4-(5-oxopyrrolidin-2-yl)phenyl)-N-(3-(4-fluoropiperidin-1-yl)propyl)-6-methoxybenzo[d]imidazo[2,1-b]thiazole-7-carboxamide, and was isolated as a yellow solid.

Yield 35 mg (17%). ¹H NMR (400 MHz, DMSO) δ 8.91 (s, 1H), 8.45 (q, J=4.4 Hz, 1H), 8.34 (s, 1H), 8.27 (t, J=5.6 Hz, 1H), 7.93 (s, 4H), 7.87 (s, 1H), 4.77-4.57 (m, 1H), 4.02 (s, 3H), 3.38-3.26 (m, 2H), 2.81 (d, J=4.4 Hz, 3H), 2.58-2.49 (m, 2H), 2.40-2.33 (m, 2H), 2.33-2.22 (m, 2H), 1.96-1.77 (m, 2H), 1.77-1.60 (m, 4H). ¹⁹F signal not observed. m/z: [ESI⁺] 524 (M+H)⁺. (C₂₇H₃₀FN₅O₃S)

### Intermediate Preparations

### Synthesis of tert-butyl 2-(3-fluoro-4-(6-((3-(4-fluoropiperidin-1-yl)propyl)carbamoyl)-7-methoxybenzo thiazolo[3,2-b][1,2,4]triazol-2-yl)phenyl)pyrrolidine-1-carboxylate

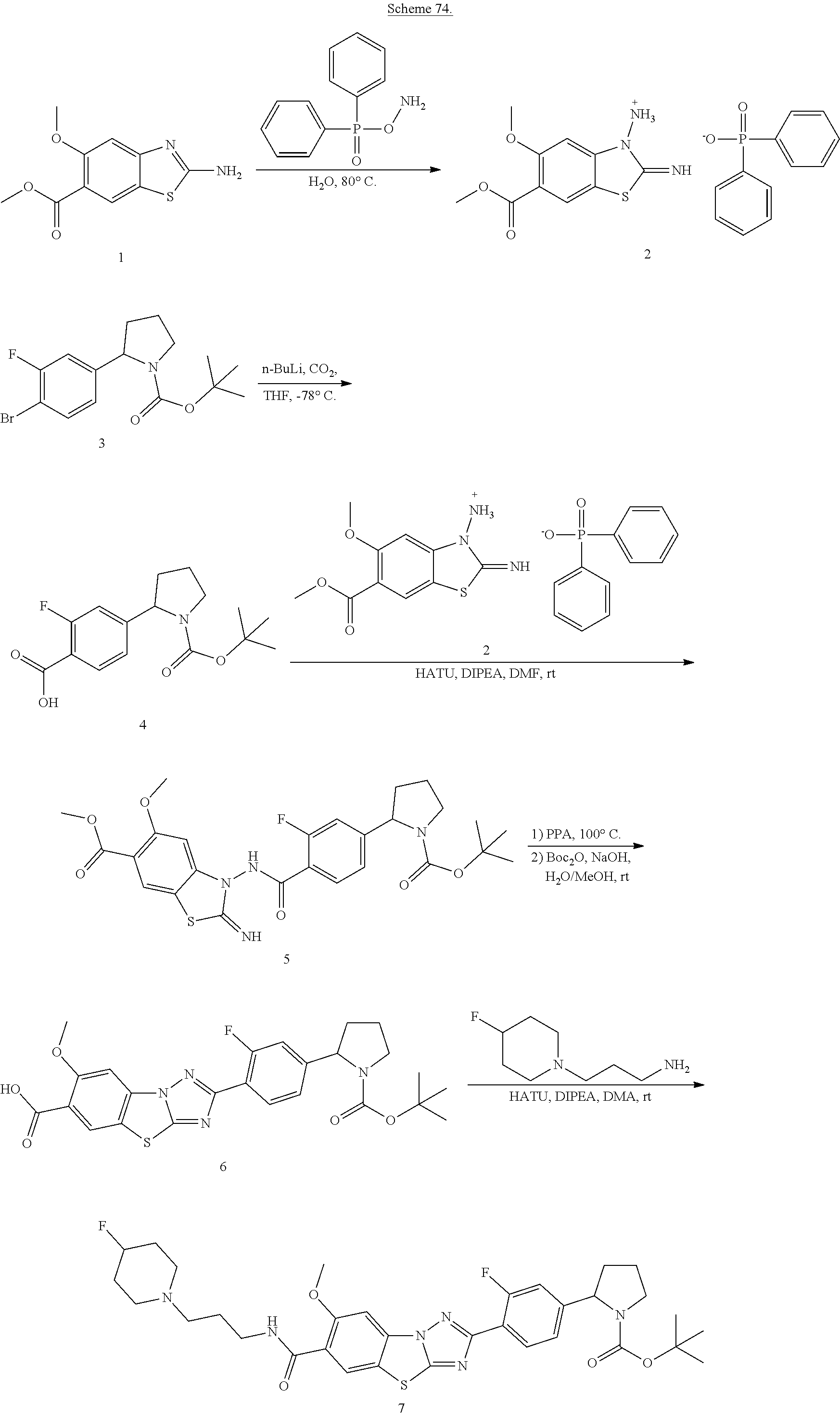

### Synthesis of 2-imino-5-methoxy-6-(methoxycarbonyl)benzo[d]thiazol-3(2H)-aminium diphenylphosphinate (2, Scheme 74)

A mixture of methyl 2-amino-5-methoxy-1,3-benzothiazole-6-carboxylate (5.00 g, 20.99 mmol) and amino diphenylphosphinate (10.00 g, 42.88 mmol) in water (60 mL) was stirred for 4 h at 80° C. under a nitrogen atmosphere. The reaction mixture was cooled to room temperature and filtered. The filter cake was washed with water (3×20 mL) and the combined filtrate was concentrated under reduced pressure. The residue was triturated with acetonitrile (60 mL) for 30 min. The precipitated solids were collected by filtration, washed with acetonitrile (3×20 mL) and oven dried to afford 2-imino-5-methoxy-6-(methoxycarbonyl)-1,3-benzothiazol-3-aminium diphenylphosphinate as a white solid. T

Yield 4.00 g (40%). ¹H NMR (300 MHz, DMSO) δ 7.93 (s, 1H), 7.71-7.62 (m, 4H), 7.38-7.25 (m, 8H), 6.96 (s, 1H), 5.68 (br s, 2H), 3.87 (s, 3H), 3.77 (s, 3H). m/z: [ESI⁺] 254 (M+H)⁺. Observed as the ammonium cationic fragment.

### Synthesis of 4-(1-(tert-butoxycarbonyl)pyrrolidin-2-yl)-2-fluorobenzoic Acid (4, Scheme 74)

A solution of tert-butyl 2-(4-bromo-3-fluorophenyl)pyrrolidine-1-carboxylate (5.60 g, 16.27 mmol) in tetrahydrofuran (70 mL) was treated with n-butyl lithium (2.5 N in hexane, 8.00 mL, 20.00 mmol) for 1 h at −78° C. under a nitrogen atmosphere. Dry ice (5.00 g, 113.61 mmol) was added as a single portion and the reaction mixture was stirred for an additional 1 h at −78° C. The reaction was quenched with saturated aqueous ammonium chloride solution (100 mL) at room temperature. The mixture was acidified to pH 5 with acetic acid and extracted with ethyl acetate (3×200 mL). The combined organic layers were washed with brine (300 mL) and dried over anhydrous sodium sulfate. After filtration, the filtrates were concentrated under reduced pressure to give 4-(1-(tert-butoxycarbonyl)pyrrolidin-2-yl)-2-fluorobenzoic acid as a colorless oil, which was used directly in the next step without further purification.

Yield 4.00 g (79%). ¹H NMR (300 MHz, DMSO) δ 12.80 (br s, 1H), 7.90-7.72 (m, 1H), 7.18-6.95 (m, 2H), 4.90-4.66 (m, 1H), 3.62-3.39 (m, 2H), 2.40-2.22 (m, 1H), 1.87-1.62 (m, 3H), 1.38-1.14 (m, 9H). m/z: [ESI⁺]254 (M+H-56)⁺.

### Synthesis of methyl 3-(4-(1-(tert-butoxycarbonyl)pyrrolidin-2-yl)-2-fluorobenzamido)-2-imino-5-methoxy-2,3-dihydrobenzo[d]thiazole-6-carboxylate (5, Scheme 74)

A solution of 4-(1-(tert-butoxycarbonyl)pyrrolidin-2-yl)-2-fluorobenzoic acid (1.00 g, 3.23 mmol) in N,N-dimethylacetamide (20 mL) was treated with O-(7-azabenzotriazol-1-yl)-N,N,N′,N′-tetramethyluronium hexafluorophosphate (HATU) (2.00 g, 5.26 mmol) for 10 min at room temperature under a nitrogen atmosphere. 2-imino-5-methoxy-6-(methoxycarbonyl)benzo[d]thiazol-3(2H)-aminium diphenylphosphinate (2.40 g, 5.09 mmol) and N,N-diisopropylethylamine (2.50 g, 19.34 mmol) were added sequentially as a single portion. The reaction mixture was stirred for 1 h. The resulting solution was purified directly by reverse phase column chromatography with the following conditions; Column: C18 Column 330 g; Mobile Phase A: water (plus 10 mmol/L ammonium bicarbonate), Mobile Phase B: acetonitrile; Flow rate: 80 mL/min; Gradient: 40%-70% B in 25 min; Detector: 254/220 nm. The fractions containing the desired product were collected at 65% B and concentrated under reduced pressure to afford methyl 3-(4-(1-(tert-butoxycarbonyl)pyrrolidin-2-yl)-2-fluorobenzamido)-2-imino-5-methoxy-2,3-dihydrobenzo[d]thiazole-6-carboxylate as a yellow solid.

Yield 1.00 g (57%). ¹H NMR (400 MHz, DMSO) δ 8.43-8.31 (m, 1H), 7.41-7.30 (m, 2H), 7.16-6.90 (m, 4H), 4.94-4.63 (m, 1H), 3.95 (s, 3H), 3.82 (s, 3H), 3.62-3.40 (m, 2H), 2.39-2.22 (m, 1H), 1.89-1.63 (m, 3H), 1.40-1.15 (m, 9H). m/z: [ESI⁺] 545 (M+H)⁺.

### Synthesis of 2-(4-(1-(tert-butoxycarbonyl)pyrrolidin-2-yl)-2-fluorophenyl)-7-methoxybenzo[4,5]thiazolo[3,2-b][1,2,4]triazole-6-carboxylic Acid (6, Scheme 74)

A mixture containing methyl 3-(4-(1-(tert-butoxycarbonyl)pyrrolidin-2-yl)-2-fluorobenzamido)-2-imino-5-methoxy-2,3-dihydrobenzo[d]thiazole-6-carboxylate (300 mg, 0.551 mmol) in polyphosphoric acid (5 mL) was stirred for 3 h at 100° C. under a nitrogen atmosphere. The reaction mixture was cooled to room temperature and quenched with ice/water (10 mL). The pH of the aqueous solution was adjusted to 9 with 3 N aqueous sodium hydroxide. Methanol (10 mL) was added, followed by di-tert-butyl dicarbonate (400 mg, 1.833 mmol) as a single portion. The reaction solution was stirred for an additional 2 h at room temperature. The pH was readjusted to 5 with 1 N aqueous hydrochloric acid. The solution was concentrated under reduced pressure to remove methanol and the resulting solid collected by vacuum filtration. After filtration, the filter cake was washed with water (1 mL) and oven dried to give 2-(4-(1-(tert-butoxycarbonyl)pyrrolidin-2-yl)-2-fluorophenyl)-7-methoxybenzo[4,5]thiazolo[3,2-b][1,2,4]triazole-6-carboxylic acid as a pale solid.

Yield 80 mg (28%). ¹H NMR not run. m/z: [ESI⁺] 513 (M+H)⁺.

### Synthesis of tert-butyl 2-(3-fluoro-4-(6-((3-(4-fluoropiperidin-1-yl)propyl)carbamoyl)-7-methoxybenzo[4,5]thiazolo[3,2-b][1,2,4]triazol-2-yl)phenyl)pyrrolidine-1-carboxylate (7, Scheme 74)

Compound tert-butyl 2-(3-fluoro-4-(6-((3-(4-fluoropiperidin-1-yl)propyl)carbamoyl)-7-methoxybenzo[4,5]thiazolo[3,2-b][1,2,4]triazol-2-yl)phenyl)pyrrolidine-1-carboxylate was prepared from 2-(4-(1-(tert-butoxycarbonyl)pyrrolidin-2-yl)-2-fluorophenyl)-7-methoxybenzo[4,5]thiazolo[3,2-b][1,2,4]triazole-6-carboxylic acid (80 mg, 0.156 mmol) and 3-(4-fluoropiperidin-1-yl)propan-1-amine (40 mg, 0.250 mmol) following a similar procedure described for the synthesis of methyl 3-(4-(1-(tert-butoxycarbonyl)pyrrolidin-2-yl)-2-fluorobenzamido)-2-imino-5-methoxy-2,3-dihydrobenzo[d]thiazole-6-carboxylate, and was isolated as a light yellow oil.

Yield 50 mg (49%). ¹H NMR (400 MHz, DMSO) δ 8.49 (s, 1H), 8.32 (t, J=5.6 Hz, 1H), 8.11 (dd, J=8.0, 8.0 Hz, 1H), 7.69 (s, 1H), 7.28-7.12 (m, 2H), 4.96-4.56 (m, 2H), 4.07 (s, 3H), 3.64-3.43 (m, 2H), 3.37-3.25 (m, 2H), 2.58-2.46 (m, 2H), 2.41-2.24 (m, 5H), 1.92-1.76 (m, 6H), 1.74-1.62 (m, 3H), 1.42-1.14 (m, 9H). m/z: [ESI⁺] 655 (M+H)⁺.

### Final Compounds

### Synthesis of 2-(2-fluoro-4-(pyrrolidin-2-yl)phenyl)-N-(3-(4-fluoropiperidin-1-yl)propyl)-7-methoxybenzo[4,5]thiazolo[3,2-b][1,2,4]triazole-6-carboxamide Dihydrochloride (Compound 178)

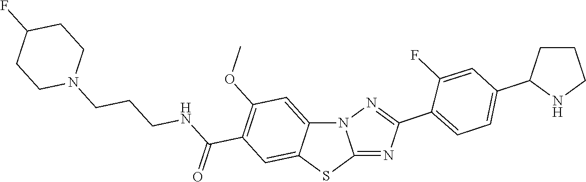 [SMILES: COc1cc2c(cc1C(=O)NCCCN1CCC(F)CC1)sc1nc(-c3ccc(C4CCCN4)cc3F)nn12]

Compound 2-(2-fluoro-4-(pyrrolidin-2-yl)phenyl)-N-(3-(4-fluoropiperidin-1-yl)propyl)-7-methoxybenzo[4,5]thiazolo[3,2-b][1,2,4]triazole-6-carboxamide dihydrochloride was prepared from tert-butyl 2-(3-fluoro-4-(6-((3-(4-fluoropiperidin-1-yl)propyl)carbamoyl)-7-methoxybenzo[4,5]thiazolo[3,2-b][1,2,4]triazol-2-yl)phenyl)pyrrolidine-1-carboxylate (50 mg, 0.076 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride, and was isolated as an off-white solid.

Yield 7 mg (15%). ¹H NMR (400 MHz, DMSO) δ 11.05 (br s, 1H), 10.48 (br s, 1H), 9.36 (br s, 1H), 8.63-8.50 (m, 2H), 8.29-8.16 (m, 1H), 7.78-7.64 (m, 2H), 7.60-7.50 (m, 1H), 5.10-4.72 (m, 1H), 4.71-4.57 (m, 1H), 4.10 (s, 3H), 3.55-3.22 (m, 6H), 3.18-2.94 (m, 4H), 2.52-2.37 (m, 1H), 2.32-1.94 (m, 9H). ¹⁹F NMR (376 MHz, DMSO) δ −110.54. Aliphatic ¹⁹F signal not observed. m/z: [ESI⁺] 555 (M+H)⁺. (C₂₈H₃₄Cl₂F₂N₆O₂S).

### Intermediate Preparations

### Synthesis of tert-butyl 2-(2,3-difluoro-4-(7-((3-(4-fluoropiperidin-1-yl)propyl)carbamoyl)-6-methoxybenzo[d]imidazo[2,1-b]thiazol-2-yl)phenyl)pyrrolidine-1-carboxylate

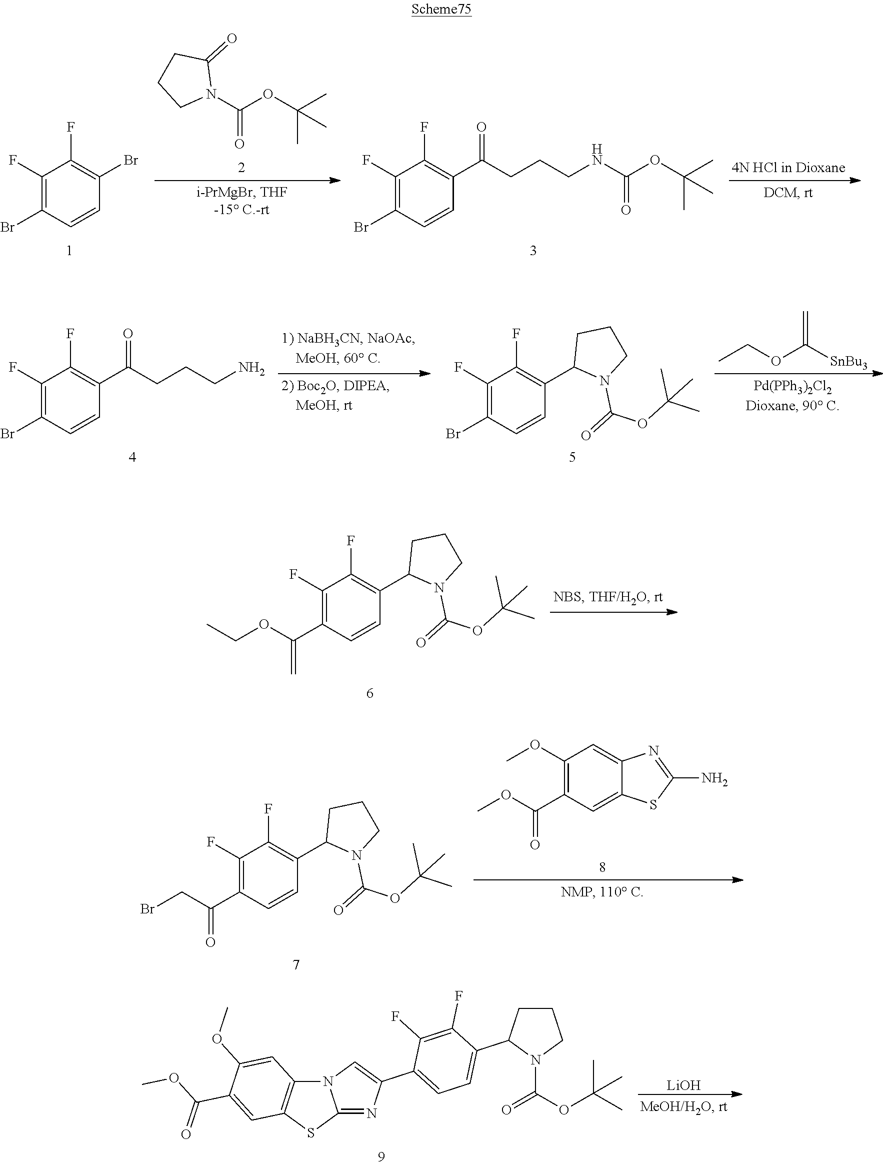

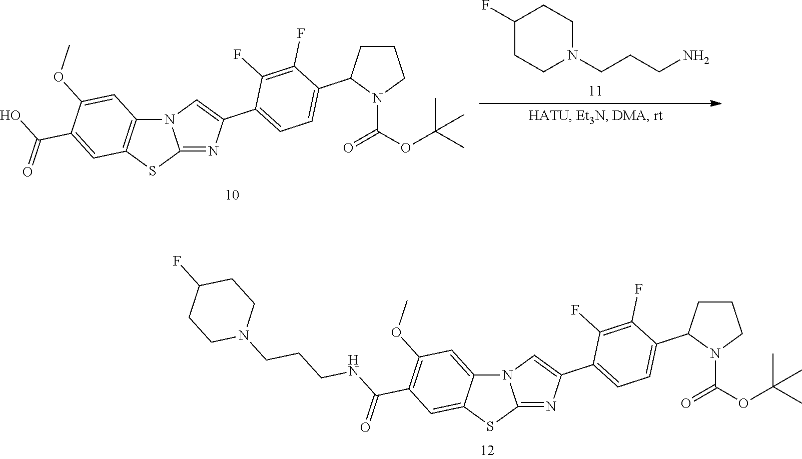 [SMILES: COc1cc2c(cc1C(=O)O)sc1nc(-c3ccc(C4CCCN4C(=O)OC(C)(C)C)c(F)c3F)cn12 ; NCCCN1CCC(F)CC1 ; COc1cc2c(cc1C(=O)NCCCN1CCC(F)CC1)sc1nc(-c3ccc(C4CCCN4C(=O)OC(C)(C)C)c(F)c3F)cn12]

### Synthesis of tert-butyl (4-(4-bromo-2,3-difluorophenyl)-4-oxobutyl)carbamate (3, Scheme 75)

Isopropylmagnesium bromide (1 N in tetrahydrofuran, 45 mL, 45.00 mmol) was added to a stirred solution of 1,4-dibromo-2,3-difluorobenzene (10.00 g, 36.78 mmol) in tetrahydrofuran (200 mL) dropwise over 5 min at −15° C. under a nitrogen atmosphere. After stirring for 30 min at this temperature, tert-butyl 2-oxopyrrolidine-1-carboxylate (7.50 g, 40.49 mmol) was added portion wise over 5 min. The reaction solution was warmed to room temperature and stirred for an additional 16 h. The reaction was quenched with saturated aqueous ammonium chloride (200 mL). The resulting mixture was extracted with ethyl acetate (3×200 mL). The combined organic layers were washed with brine (300 mL) and dried over anhydrous sodium sulfate. After filtration, the filtrate was concentrated under reduced pressure. The residue was purified by silica gel column chromatography (0-20% ethyl acetate in petroleum ether) to afford tert-butyl (4-(4-bromo-2,3-difluorophenyl)-4-oxobutyl)carbamate as a yellow oil.

Yield 9.32 g (67%). ¹H NMR (400 MHz, CDCl₃) δ 7.59-7.52 (m, 1H), 7.48-7.40 (m, 1H), 4.61 (br s, 1H), 3.28-3.17 (m, 2H), 3.07-2.97 (m, 2H), 2.00-1.88 (m, 2H), 1.45 (s, 9H). m/z: [ESI⁺] 378, 380 (M+H)⁺.

### Synthesis of 4-amino-1-(4-bromo-2,3-difluorophenyl)butan-1-one hydrochloride (4, Scheme 75)

tert-butyl (4-(4-bromo-2,3-difluorophenyl)-4-oxobutyl)carbamate (9.32 g, 24.64 mmol) in a solution of hydrochloride acid in 1,4-dioxane (4 N, 50 mL) and dichloromethane (50 mL) was stirred for 1 h at room temperature. The precipitated solids were collected by filtration, washed with dichloromethane (3×10 mL) and dried in a vacuum oven to afford 4-amino-1-(4-bromo-2,3-difluorophenyl)butan-1-one hydrochloride as a white solid.

Yield 6.00 g (77%). ¹H NMR (300 MHz, DMSO) δ 8.00 (br s, 3H), 7.75-7.60 (m, 2H), 3.20-3.10 (m, 2H), 2.91-2.78 (m, 2H), 1.98-1.86 (m, 2H). m/z: [ESI⁺] 260, 262 (M+H−18)⁺.

### Synthesis of tert-butyl 2-(4-bromo-2,3-difluorophenyl)pyrrolidine-1-carboxylate (5, Scheme 75)

A solution of 4-amino-1-(4-bromo-2,3-difluorophenyl)butan-1-one hydrochloride (5.89 g, 18.73 mmol) in methanol (150 mL) was treated with sodium acetate (5.20 g, 63.39 mmol) for 16 h at 60° C. The reaction mixture was cooled to room temperature and treated with sodium cyanoborohydride (2.66 g, 42.33 mmol), which was added portion wise over 2 min. The reaction mixture was heated to 60° C. and stirred for an additional 5 h. The reaction mixture was cooled to room temperature and concentrated under reduced pressure. The residue was triturated with ethyl acetate (200 mL) for 30 min. After filtration, the filtrate was concentrated under reduced pressure again. The residue was dissolved in methanol (100 mL). To the above solution were added N,N-diisopropylethylamine (12 mL, 68.89 mmol) and di-tert-butyl dicarbonate (7.00 g, 32.07 mmol) sequentially at 0° C. The reaction solution was stirred for an additional 2 h at room temperature. The reaction mixture was concentrated under reduced pressure. The residue was triturated with ethyl acetate (100 mL) for 10 min. After filtration, the filtered cake was washed with dichloromethane (3×10 mL). The combined filtrates were concentrated under reduced pressure. The residue was purified by silica gel column chromatography (0-30% ethyl acetate in petroleum ether) to afford tert-butyl 2-(4-bromo-2,3-difluorophenyl)pyrrolidine-1-carboxylate as a light yellow oil.

Yield 6.60 g (97%). ¹H NMR (400 MHz, CDCl₃) δ 7.30-7.18 (m, 1H), 6.89-6.74 (m, 1H), 5.18-5.00 (m, 1H), 3.72-3.48 (m, 2H), 2.47-2.28 (m, 1H), 1.97-1.79 (m, 3H), 1.52-1.19 (m, 9H). m/z: [ESI⁺] 306, 308 (M+H−56)⁺.

### Synthesis of tert-butyl 2-(4-(1-ethoxyvinyl)-2, 3-difluorophenyl)pyrrolidine-1-carboxylate (6, Scheme 75)

Compound tert-butyl 2-(4-(1-ethoxyvinyl)-2,3-difluorophenyl)pyrrolidine-1-carboxylate was prepared from tert-butyl 2-(4-bromo-2,3-difluorophenyl)pyrrolidine-1-carboxylate (3.00 g, 8.28 mmol) and tributyl(1-ethoxyvinyl)stannane (3.60 g, 9.97 mmol) following a similar procedure to that described for the synthesis of tert-butyl (S)-2-(4-(1-ethoxyvinyl)-3-fluorophenyl)pyrrolidine-1-carboxylate, and was isolated as a brown oil. The crude product was used directly in the next step without further purification.

Yield 2.90 g (crude). ¹H NMR not run. m/z: [ESI⁺] 298 (M+H−56)⁺.

### Synthesis of tert-butyl 2-(4-(2-bromoacetyl)-2,3-difluorophenyl)pyrrolidine-1-carboxylate (7, Scheme 75)

Compound tert-butyl 2-(4-(2-bromoacetyl)-2,3-difluorophenyl)pyrrolidine-1-carboxylate was prepared from tert-butyl 2-(4-(1-ethoxyvinyl)-2,3-difluorophenyl)pyrrolidine-1-carboxylate (2.90 g, crude) and N-bromosuccinimide (1.45 g, 8.15 mmol) following a similar procedure to that described for the synthesis of tert-butyl (S)-2-(4-(2-bromoacetyl)-3-fluorophenyl)pyrrolidine-1-carboxylate, and was isolated as a yellow oil.

Yield 2.85 g (85% over 2 steps). ¹H NMR (300 MHz, CDCl₃) δ 7.72-7.62 (m, 1H), 7.13-6.99 (m, 1H), 5.25-5.05 (m, 1H), 4.51 (s, 2H), 3.72-3.52 (m, 2H), 2.52-2.34 (m, 1H), 2.01-1.79 (m, 3H), 1.51-1.18 (m, 9H). m/z: [ESI⁺] 404, 406 (M+H−56)⁺.

### Synthesis of methyl 2-(4-(1-(tert-butoxycarbonyl)pyrrolidin-2-yl)-2,3-difluorophenyl)-6-methoxybenzo[d]imidazo[2,1-b]thiazole-7-carboxylate (9, Scheme 75)

Compound methyl 2-(4-(1-(tert-butoxycarbonyl)pyrrolidin-2-yl)-2,3-difluorophenyl)-6-methoxybenzo[d]imidazo[2,1-b]thiazole-7-carboxylate was prepared from tert-butyl 2-(4-(2-bromoacetyl)-2,3-difluorophenyl)pyrrolidine-1-carboxylate (1.00 g, 2.47 mmol) and methyl 2-amino-5-methoxybenzo[d]thiazole-6-carboxylate (0.59 g, 2.48 mmol) following a similar procedure to that described for the synthesis of tert-butyl (S)-2-(4-(7-bromo-9-methyl-9H-benzo[d]imidazo[1,2-a]imidazol-2-yl)-3-fluorophenyl)pyrrolidine-1-carboxylate, and was isolated as a yellow solid.

Yield 0.60 g (45%). ¹H NMR (400 MHz, DMSO) δ 8.85 (d, J=3.6 Hz, 1H), 8.59 (s, 1H), 8.11 (s, 1H), 7.92-7.81 (m, 1H), 7.25-7.10 (m, 1H), 5.13-4.94 (m, 1H), 3.92 (s, 3H), 3.87 (s, 3H), 3.62-3.43 (m, 2H), 2.46-2.28 (m, 1H), 1.95-1.70 (m, 3H), 1.43-1.12 (m, 9H). m/z: [ESI⁺] 544 (M+H)⁺.

### Synthesis of 2-(4-(1-(tert-butoxycarbonyl)pyrrolidin-2-yl)-2, 3-difluorophenyl)-6-methoxybenzo[d]imidazo[2,1-b]thiazole-7-carboxylic acid (10, Scheme 75)

Compound 2-(4-(1-(tert-butoxycarbonyl)pyrrolidin-2-yl)-2,3-difluorophenyl)-6-methoxybenzo[d]imidazo[2,1-b]thiazole-7-carboxylic acid was prepared at room temperature from methyl 2-(4-(1-(tert-butoxycarbonyl)pyrrolidin-2-yl)-2,3-difluorophenyl)-6-methoxybenzo[d]imidazo[2,1-b]thiazole-7-carboxylate (0.60 g, 1.10 mmol) following a similar procedure to that described for the synthesis of 2-(1-(tert-butoxycarbonyl)-6-fluoroindolin-5-yl)benzo[d]imidazo[2,1-b]thiazole-7-carboxylic acid and was isolated as a yellow solid.

Yield 0.45 g (77%). ¹H NMR (400 MHz, CDCl₃) δ 8.87 (s, 1H), 8.51 (s, 1H), 7.96-7.85 (m, 1H), 7.81 (s, 1H), 7.18-7.07 (m, 1H), 5.33-5.02 (m, 1H), 4.19 (s, 3H), 3.73-3.46 (m, 2H), 2.56-2.29 (m, 1H), 1.99-1.77 (m, 3H), 1.56-1.16 (m, 9H). OH proton not observed. m/z: [ESI⁺] 530 (M+H)⁺.

### Synthesis of tert-butyl 2-(2,3-difluoro-4-(7-((3-(4-fluoropiperidin-1-yl)propyl)carbamoyl)-6-methoxybenzo[d]imidazo[2,1-b]thiazol-2-yl)phenyl)pyrrolidine-1-carboxylate (12, Scheme 75)

Compound tert-butyl 2-(2,3-difluoro-4-(7-((3-(4-fluoropiperidin-1-yl)propyl)carbamoyl)-6-methoxybenzo[d]imidazo[2,1-b]thiazol-2-yl)phenyl)pyrrolidine-1-carboxylate was prepared from 2-(4-(1-(tert-butoxycarbonyl)pyrrolidin-2-yl)-2,3-difluorophenyl)-6-methoxybenzo[d]imidazo[2,1-b]thiazole-7-carboxylic acid (0.45 g, 0.85 mmol) and 3-(4-fluoropiperidin-1-yl)propan-1-amine (0.30 g, 1.87 mmol) following a similar procedure to that described for the synthesis of tert-butyl 6-fluoro-5-(7-((3-(4-fluoropiperidin-1-yl)propyl)carbamoyl)benzo[d]imidazo[2,1-b]thiazol-2-yl)indoline-1-carboxylate and was isolated as a yellow solid.

Yield 0.10 g (18%). ¹H NMR (400 MHz, CDCl₃) δ 8.57 (s, 1H), 8.20-8.13 (m, 1H), 7.98-7.89 (m, 1H), 7.25 (s, 1H), 7.09-6.92 (m, 1H), 5.28-5.05 (m, 1H), 4.95-4.74 (m, 1H), 4.19 (s, 3H), 3.72-3.57 (m, 6H), 3.03-2.53 (m, 4H), 2.51-2.30 (m, 1H), 2.02-1.86 (m, 9H), 1.50-1.22 (m, 9H). NH not observed. m/z: [ESI⁺] 672 (M+H)⁺.

### Synthesis of tert-butyl 3-(4-(7-carbamoyl-6-methoxybenzo[d]imidazo[2,1-b]thiazol-2-yl)-3-fluorophenyl)pyrrolidine-1-carboxylate

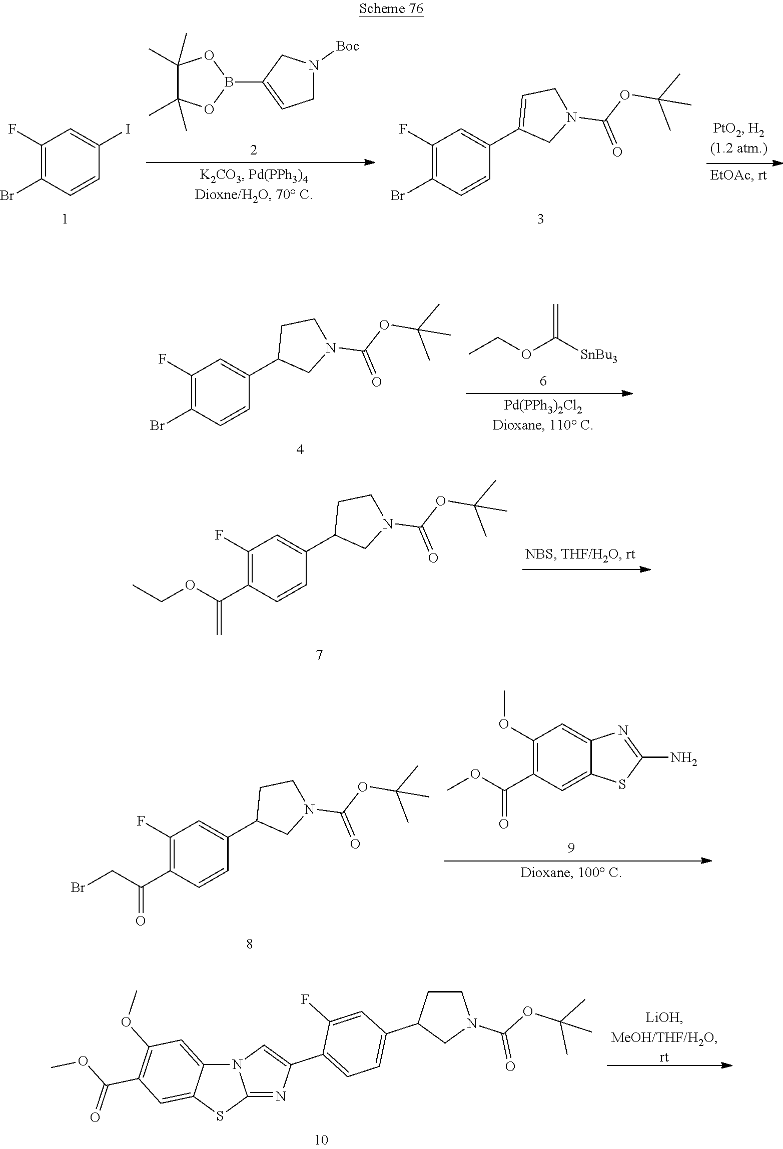

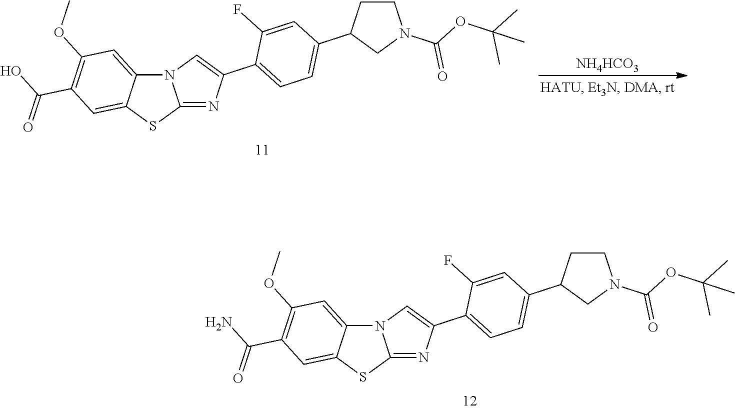 [SMILES: COc1cc2c(cc1C(=O)O)sc1nc(-c3ccc(C4CCN(C(=O)OC(C)(C)C)C4)cc3F)cn12 ; "NH4HCO3" ; COc1cc2c(cc1C(N)=O)sc1nc(-c3ccc(C4CCN(C(=O)OC(C)(C)C)C4)cc3F)cn12]

### Synthesis of tert-butyl 3-(4-bromo-3-fluorophenyl)-2,5-dihydro-1H-pyrrole-1-carboxylate (3, Scheme 76)

Compound tert-butyl 3-(4-bromo-3-fluorophenyl)-2,5-dihydro-1H-pyrrole-1-carboxylate was prepared from 1-bromo-2-fluoro-4-iodobenzene (10.19 g, 33.87 mmol) and tert-butyl 3-(4,4,5,5-tetramethyl-1,3,2-dioxaborolan-2-yl)-2,5-dihydro-1H-pyrrole-1-carboxylate (10.00 g, 33.88 mmol) following a similar procedure to that described for the synthesis of tert-butyl 4-(4-bromo-3-fluorophenyl)-3,6-dihydropyridine-1(2H)-carboxylate and was isolated as a yellow solid.

Yield 6.50 g (56%). ¹H NMR (300 MHz, CDCl₃) δ 7.54 (dd, J=7.2, 8.4 Hz, 1H), 7.15 (dd, J=2.1, 9.6 Hz, 1H), 7.05 (dd, J=2.1, 8.4 Hz, 1H), 6.26-6.15 (m, 1H), 4.53-4.40 (m, 2H), 4.38-4.26 (m, 2H), 1.53 (s, 9H). m/z: [ESI⁺] 286, 288 (M+H−56)⁺.

### Synthesis of tert-butyl 3-(4-bromo-3-fluorophenyl)pyrrolidine-1-carboxylate (4, Scheme 76)

Compound tert-butyl 3-(4-bromo-3-fluorophenyl)pyrrolidine-1-carboxylate was prepared from tert-butyl 3-(4-bromo-3-fluorophenyl)-2,5-dihydro-1H-pyrrole-1-carboxylate (6.50 g, 18.99 mmol) following a similar procedure to that described for the synthesis of tert-butyl 4-(4-bromo-3-fluorophenyl)piperidine-1-carboxylate and was isolated as a colorless oil.

Yield 2.60 g (40%). ¹H NMR (300 MHz, CDCl₃) δ 7.50 (dd, J=7.2, 8.4 Hz, 1H), 7.03 (dd, J=2.1, 9.6 Hz, 1H), 6.93 (dd, J=2.1, 8.4 Hz, 1H), 3.87-3.78 (m, 1H), 3.67-3.55 (m, 1H), 3.52-3.18 (m, 3H), 2.35-2.22 (m, 1H), 2.04-1.89 (m, 1H), 1.50 (s, 9H). m/z: [ESI⁺] 288, 290 (M+H−56)⁺.

### Synthesis of tert-butyl 3-(4-(1-ethoxyvinyl)-3-fluorophenyl)pyrrolidine-1-carboxylate (7, Scheme 76)

Compound tert-butyl 3-(4-(1-ethoxyvinyl)-3-fluorophenyl)pyrrolidine-1-carboxylate was prepared from tert-butyl 3-(4-bromo-3-fluorophenyl)pyrrolidine-1-carboxylate (1.00 g, 2.91 mmol) and tributyl(1-ethoxyvinyl)stannane (1.50 g, 4.15 mmol) following a similar procedure to that described for the synthesis of tert-butyl (S)-2-(4-(1-ethoxyvinyl)-3-fluorophenyl)pyrrolidine-1-carboxylate and was isolated as a brown oil. The crude product was used directly in the next step without further purification.

Yield 1.00 g (crude). ¹H NMR not run. m/z: [ESI⁺] 336 (M+H)⁺.

### Synthesis of tert-butyl 3-(4-(2-bromoacetyl)-3-fluorophenyl)pyrrolidine-1-carboxylate (8, Scheme 76)

Compound tert-butyl 3-(4-(2-bromoacetyl)-3-fluorophenyl)pyrrolidine-1-carboxylate was prepared from tert-butyl 3-(4-(1-ethoxyvinyl)-3-fluorophenyl)pyrrolidine-1-carboxylate (1.00 g, crude) and N-bromosuccinimide (0.60 g, 3.37 mmol) following a similar procedure to that described for the synthesis of tert-butyl (S)-2-(4-(2-bromoacetyl)-3-fluorophenyl)pyrrolidine-1-carboxylate and was isolated as a yellow oil.

Yield 1.00 g (89% over two steps). ¹H NMR (300 MHz, DMSO) δ 7.91-7.82 (m, 1H), 7.39-7.28 (m, 2H), 4.80 (s, 2H), 3.77-3.68 (m, 1H), 3.53-3.40 (m, 2H), 3.38-3.26 (m, 1H), 3.26-3.14 (m, 1H), 2.31-2.16 (m, 1H), 2.09-1.91 (m, 1H), 1.42 (s, 9H). m/z: [ESI⁺] 330, 332 (M+H−56)⁺.

### Synthesis of methyl 2-(4-(1-(tert-butoxycarbonyl)pyrrolidin-3-yl)-2-fluorophenyl)-6-methoxybenzo[d]imidazo[2,1-b]thiazole-7-carboxylate (10, Scheme 76)

Compound methyl 2-(4-(1-(tert-butoxycarbonyl)pyrrolidin-3-yl)-2-fluorophenyl)-6-methoxybenzo[d]imidazo[2,1-b]thiazole-7-carboxylate was prepared from tert-butyl 3-(4-(2-bromoacetyl)-3-fluorophenyl)pyrrolidine-1-carboxylate (1.00 g, 2.59 mmol) and methyl 2-amino-5-methoxybenzo[d]thiazole-6-carboxylate (1.00 g, 4.20 mmol) following a similar procedure to that described for the synthesis of tert-butyl (S)-2-(4-(7-bromo-9-methyl-9H-benzo[d]imidazo[1,2-a]imidazol-2-yl)-3-fluorophenyl)pyrrolidine-1-carboxylate and was isolated as a brown solid.

Yield 0.70 g (51%). ¹H NMR not run. m/z: [ESI⁺] 526 (M+H)⁺.

### Synthesis of 2-(4-(1-(tert-butoxycarbonyl)pyrrolidin-3-yl)-2-fluorophenyl)-6-methoxybenzo[d]imidazo[2,1-b]thiazole-7-carboxylic Acid (11, Scheme 76)

Compound 2-(4-(1-(tert-butoxycarbonyl)pyrrolidin-3-yl)-2-fluorophenyl)-6-methoxybenzo[d]imidazo[2,1-b]thiazole-7-carboxylic acid was prepared from methyl 2-(4-(1-(tert-butoxycarbonyl)pyrrolidin-3-yl)-2-fluorophenyl)-6-methoxybenzo[d]imidazo[2,1-b]thiazole-7-carboxylate (0.70 g, 1.33 mmol) at room temperature following a similar procedure to that described for the synthesis of 2-(1-(tert-butoxycarbonyl)-6-fluoroindolin-5-yl)benzo[d]imidazo[2,1-b]thiazole-7-carboxylic acid and was isolated as an off-white solid.

Yield 0.60 g (88%). ¹H NMR (400 MHz, DMSO) δ 8.65 (d, J=3.6 Hz, 1H), 8.13-8.04 (m, 1H), 7.81 (s, 1H), 7.75 (s, 1H), 7.31-7.25 (m, 1H), 7.25-7.20 (m, 1H), 3.85 (s, 3H), 3.78-3.69 (m, 1H), 3.53-3.44 (m, 1H), 3.44-3.35 (m, 1H), 3.34-3.26 (m, 1H), 3.24-3.19 (m, 1H), 2.30-2.16 (m, 1H), 2.07-1.95 (m, 1H), 1.43 (s, 9H). OH proton not observed. m/z: [ESI⁺] 512 (M+H)⁺.

### Synthesis of tert-butyl 3-(4-(7-carbamoyl-6-methoxybenzo[d]imidazo[2,1-b]thiazol-2-yl)-3-fluorophenyl)pyrrolidine-1-carboxylate (12, Scheme 76)

Compound tert-butyl 3-(4-(7-carbamoyl-6-methoxybenzo[d]imidazo[2,1-b]thiazol-2-yl)-3-fluorophenyl)pyrrolidine-1-carboxylate was prepared from 2-(4-(1-(tert-butoxycarbonyl)pyrrolidin-3-yl)-2-fluorophenyl)-6-methoxybenzo[d]imidazo[2,1-b]thiazole-7-carboxylic acid (150 mg, 0.293 mmol) and ammonium bicarbonate (40 mg, 0.506 mmol) following a similar procedure to that described for the synthesis of tert-butyl 6-fluoro-5-(7-((3-(4-fluoropiperidin-1-yl)propyl)carbamoyl)benzo[d]imidazo[2,1-b]thiazol-2-yl)indoline-1-carboxylate and was isolated as an off-white solid.

Yield 70 mg (47%). ¹H NMR (300 MHz, DMSO) δ 8.74 (d, J=3.6 Hz, 1H), 8.42 (s, 1H), 8.15-8.04 (m, 2H), 7.74 (br s, 1H), 7.64 (br s, 1H), 7.35-7.21 (m, 2H), 4.04 (s, 3H), 3.81-3.69 (m, 1H), 3.59-3.34 (m, 3H), 3.27-3.16 (m, 1H), 2.31-2.16 (m, 1H), 2.09-1.92 (m, 1H), 1.43 (s, 9H). m/z: [ESI⁺] 511 (M+H)⁺.

### Synthesis of tert-butyl 3-(3-fluoro-4-(6-methoxy-7-(methylcarbamoyl)benzo[d]imidazo[2,1-b]thiazol-2-yl)phenyl)pyrrolidine-1-carboxylate

Compound tert-butyl 3-(3-fluoro-4-(6-methoxy-7-(methylcarbamoyl)benzo[d]imidazo[2,1-b]thiazol-2-yl)phenyl)pyrrolidine-1-carboxylate was prepared from 2-(4-(1-(tert-butoxycarbonyl)pyrrolidin-3-yl)-2-fluorophenyl)-6-methoxybenzo[d]imidazo[2,1-b]thiazole-7-carboxylic acid (150 mg, 0.293 mmol) and methanamine hydrochloride (40 mg, 0.592 mmol) following a similar procedure to that described for the synthesis of tert-butyl 6-fluoro-5-(7-((3-(4-fluoropiperidin-1-yl)propyl)carbamoyl)benzo[d]imidazo[2,1-b]thiazol-2-yl)indoline-1-carboxylate and was isolated as a brown solid.

Yield 80 mg (52%). ¹H NMR (300 MHz, DMSO) δ 8.72 (d, J=3.6 Hz, 1H), 8.37 (s, 1H), 8.23 (q, J 4.8 Hz, 1H), 8.14-8.03 (m, 2H), 7.35-7.18 (m, 2H), 4.03 (s, 3H), 3.80-3.67 (m, 1H), 3.55-3.26 (m, 3H), 3.25-3.15 (m, 1H), 2.84 (d, J=4.8 Hz, 3H), 2.32-2.17 (m, 1H), 2.11-1.94 (m, 1H), 1.43 (s, 9H). m/z: [ESI⁺] 525 (M+H)⁺.

### Synthesis of tert-butyl 3-(3-fluoro-4-(6-methoxy-7-((1-methylpiperidin-4-yl)carbamoyl)benzo[d]imidazo[2,1-b]thiazol-2-yl)phenyl)pyrrolidine-1-carboxylate

Compound tert-butyl 3-(3-fluoro-4-(6-methoxy-7-((1-methylpiperidin-4-yl)carbamoyl)benzo[d]imidazo[2,1-b]thiazol-2-yl)phenyl)pyrrolidine-1-carboxylate was prepared from 2-(4-(1-(tert-butoxycarbonyl)pyrrolidin-3-yl)-2-fluorophenyl)-6-methoxybenzo[d]imidazo[2,1-b]thiazole-7-carboxylic acid (150 mg, 0.293 mmol) and 1-methylpiperidin-4-amine (50 mg, 0.438 mmol) following a similar procedure to that described for the synthesis of tert-butyl 6-fluoro-5-(7-((3-(4-fluoropiperidin-1-yl)propyl)carbamoyl)benzo[d]imidazo[2,1-b]thiazol-2-yl)indoline-1-carboxylate and was isolated as an orange solid.

Yield 100 mg (56%). ¹H NMR (300 MHz, DMSO) δ 8.72 (d, J=3.6 Hz, 1H), 8.28 (s, 1H), 8.13-8.02 (m, 3H), 7.31-7.20 (m, 2H), 4.02 (s, 3H), 3.83-3.69 (m, 2H), 3.56-3.26 (m, 4H), 3.26-3.16 (m, 1H), 2.75-2.64 (m, 2H), 2.31-2.17 (m, 4H), 2.11-1.93 (m, 3H), 1.90-1.76 (m, 2H), 1.65-1.48 (m, 1H), 1.43 (s, 9H). m/z: [ESI⁺] 608 (M+H)⁺.

### Synthesis of tert-butyl 3-(3-fluoro-4-(7-((3-(4-fluoropiperidin-1-yl)propyl)carbamoyl)-6-methoxybenzo[d]imidazo[2,1-b]thiazol-2-yl)phenyl)pyrrolidine-1-carboxylate

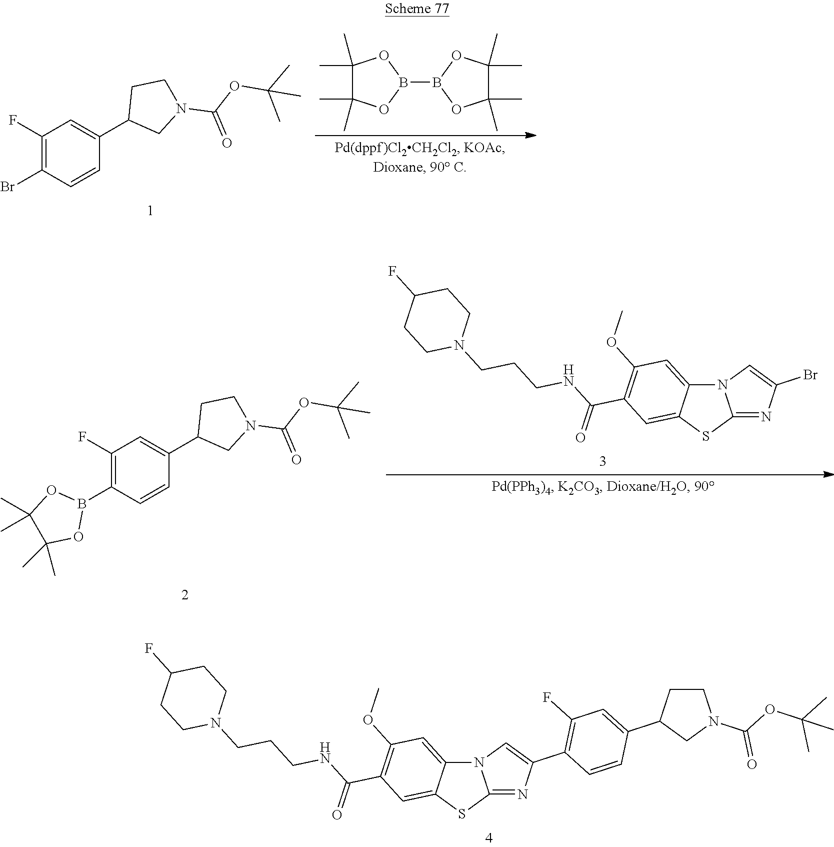

### Synthesis of tert-butyl 3-(3-fluoro-4-(4,4,5,5-tetramethyl-1,3,2-dioxaborolan-2-yl)phenyl)pyrrolidine-1-carboxylate (2, Scheme 77)

To a stirred mixture of tert-butyl 3-(4-bromo-3-fluorophenyl)pyrrolidine-1-carboxylate (300 mg, 0.872 mmol) and bis(pinacolato)diboron (330 mg, 1.300 mmol) in 1,4-dioxane (10 mL), were added potassium acetate (260 mg, 2.649 mmol) and 1,1′-bis(diphenylphosphino)ferrocene-palladium(II)dichloride dichloromethane complex (70 mg, 0.086 mmol) sequentially as a single portion at room temperature under a nitrogen atmosphere. The reaction mixture was stirred for 16 h at 90° C. The mixture was cooled to room temperature, diluted with water (20 mL) and extracted with ethyl acetate (3×20 mL). The combined organic extracts were concentrated under reduced pressure to afford tert-butyl 3-(3-fluoro-4-(4,4,5,5-tetramethyl-1,3,2-dioxaborolan-2-yl)phenyl)pyrrolidine-1-carboxylate as a brown solid, which was used directly in the next step without further purification.

Yield 250 mg (crude). ¹H NMR not run. m/z: [ESI⁺] 336 (M+H−56)⁺.

### Synthesis of tert-butyl 3-(3-fluoro-4-(7-((3-(4-fluoropiperidin-1-yl)propyl)carbamoyl)-6-methoxybenzo[d]imidazo[2,1-b]thiazol-2-yl)phenyl)pyrrolidine-1-carboxylate (4, Scheme 77)

Compound tert-butyl 3-(3-fluoro-4-(7-((3-(4-fluoropiperidin-1-yl)propyl)carbamoyl)-6-methoxybenzo[d]imidazo[2,1-b]thiazol-2-yl)phenyl)pyrrolidine-1-carboxylate was prepared from 2-bromo-N-(3-(4-fluoropiperidin-1-yl)propyl)-6-methoxybenzo[d]imidazo[2,1-b]thiazole-7-carboxamide (200 mg, 0.426 mmol) and tert-butyl 3-(3-fluoro-4-(4,4,5,5-tetramethyl-1,3,2-dioxaborolan-2-yl)phenyl)pyrrolidine-1-carboxylate (250 mg, crude) at 90° C., following a similar procedure to that described for the synthesis of tert-butyl 4-(4-bromo-3-fluorophenyl)-3,6-dihydropyridine-1(2H)-carboxylate and was isolated as a yellow solid.

Yield 80 mg (29%). ¹H NMR (400 MHz, DMSO) δ 8.74 (d, J=3.6 Hz, 1H), 8.33 (s, 1H), 8.28 (t, J 5.6 Hz, 1H), 8.15-8.03 (m, 2H), 7.36-7.27 (m, 1H), 7.27-7.20 (m, 1H), 4.78-4.57 (m, 1H), 4.02 (s, 3H), 3.80-3.68 (m, 1H), 3.55-3.26 (m, 6H), 3.25-3.14 (m, 1H), 2.42-2.14 (m, 6H), 2.07-1.94 (m, 1H), 1.92-1.76 (m, 2H), 1.76-1.60 (m, 4H), 1.43 (s, 9H). m/z: [ESI⁺] 654 (M+H)⁺.

### Synthesis of tert-butyl 4-(3-fluoro-4-(7-((3-(4-fluoropiperidin-1-yl)propyl)carbamoyl)-6-methoxybenzo[d]imidazo[2,1-b]thiazol-2-yl)phenyl)piperidine-1-carboxylate

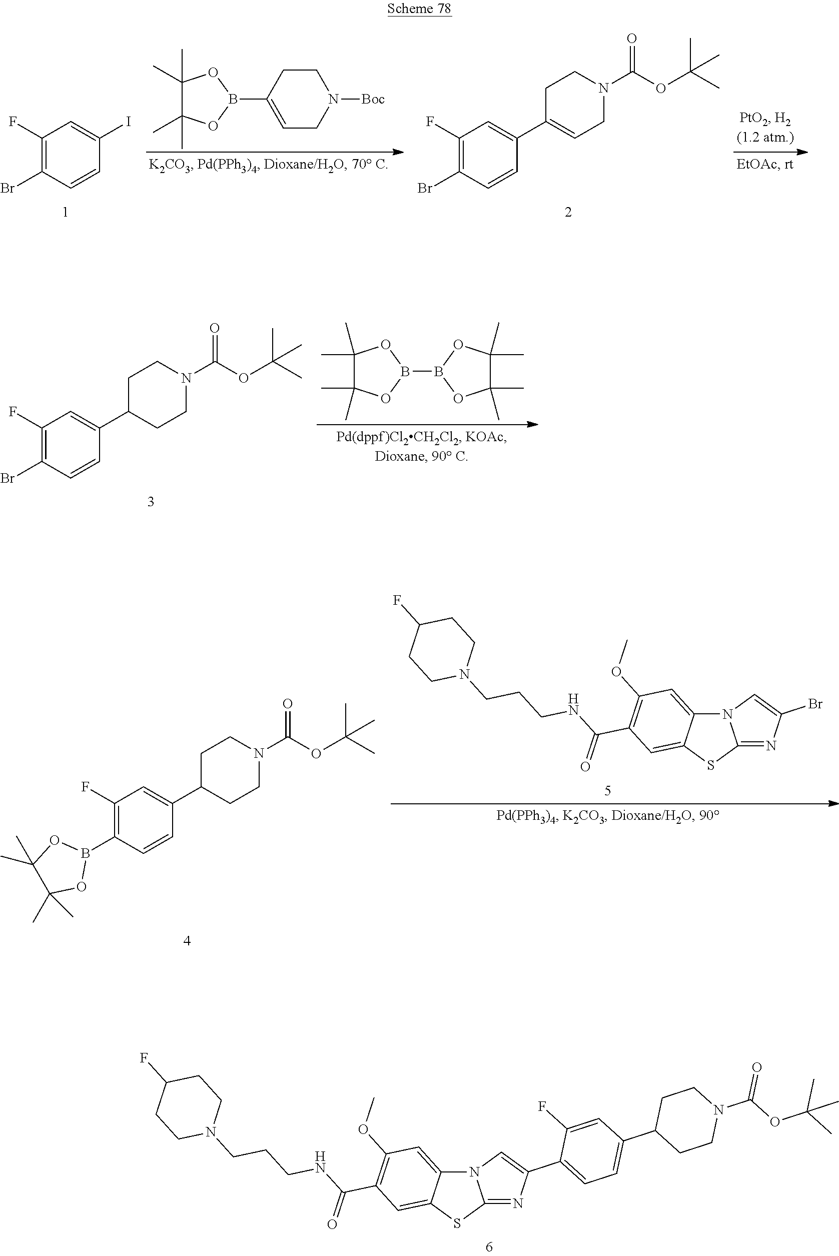

### Synthesis of tert-butyl 4-(4-bromo-3-fluorophenyl)-3,6-dihydropyridine-1(2H)-carboxylate (2, Scheme 78)

To a stirred mixture of 1-bromo-2-fluoro-4-iodobenzene (15.00 g, 49.85 mmol) and tert-butyl 4-(4,4,5,5-tetramethyl-1,3,2-dioxaborolan-2-yl)-3,6-dihydropyridine-1(2H)-carboxylate (18.00 g, 58.21 mmol) in 1,4-dioxane:water (7:1, 170 mL), were added potassium carbonate (21.00 g, 151.95 mmol) and tetrakis(triphenylphosphine)palladium (0) (5.70 g, 4.93 mmol) at room temperature under a nitrogen atmosphere. The reaction mixture was stirred for 4 h at 70° C. The mixture was cooled to room temperature, diluted with water (500 mL) and extracted with ethyl acetate (3×300 mL). The combined organic layers were washed with brine (300 mL) and dried over anhydrous sodium sulfate. After filtration, the filtrate was concentrated under reduced pressure. The residue was purified by silica gel column chromatography (25% ethyl acetate in petroleum ether) to afford tert-butyl 4-(4-bromo-3-fluorophenyl)-3,6-dihydropyridine-1(2H)-carboxylate as a white oil.

Yield 16.00 g (90%). ¹H NMR (400 MHz, CD₃OD) δ 7.56 (dd, J=7.2, 8.4 Hz, 1H), 7.28 (dd, J=2.0, 10.4 Hz, 1H), 7.18 (dd, J=2.0, 8.4 Hz, 1H), 6.25-6.15 (m, 1H), 4.12-4.01 (m, 2H), 3.67-3.59 (m, 2H), 2.55-2.48 (m, 2H), 1.50 (s, 9H). m/z: [ESI⁺] 300, 302 (M+H−56)⁺.

### Synthesis of tert-butyl 4-(4-bromo-3-fluorophenyl)piperidine-1-carboxylate (3, Scheme 78)

A mixture of tert-butyl 4-(4-bromo-3-fluorophenyl)-3,6-dihydropyridine-1(2H)-carboxylate (10.00 g, 28.07 mmol) and platinum dioxide (1.00 g, 4.40 mmol) in ethyl acetate (15 mL) was stirred for 2 h at room temperature under a hydrogen atmosphere (1.2 atm.). After filtration, the filter cake was washed with ethyl acetate (3×50 mL). The combined filtrates were concentrated under reduced pressure. The residue was purified by silica gel column chromatography (30% ethyl acetate in petroleum ether) to afford tert-butyl 4-(4-bromo-3-fluorophenyl)piperidine-1-carboxylate as a colorless oil.

Yield 8.00 g (80%). ¹H NMR (400 MHz, DMSO) δ 7.61 (dd, J=7.2, 8.4 Hz, 1H), 7.30 (dd, J=2.0, 10.4 Hz, 1H), 7.08 (dd, J=2.0, 8.4 Hz, 1H), 4.12-4.01 (m, 2H), 2.86-2.65 (m, 3H), 1.79-1.69 (m, 2H), 1.53-1.43 (m, 2H), 1.42 (s, 9H). m/z: [ESI⁺] 302, 304 (M+H−56)⁺.

### Synthesis of tert-butyl 4-(3-fluoro-4-(4,4,5,5-tetramethyl-1,3,2-dioxaborolan-2-yl)phenyl)piperidine-1-carboxylate (4, Scheme 78)

Compound tert-butyl 4-(3-fluoro-4-(4,4,5,5-tetramethyl-1,3,2-dioxaborolan-2-yl)phenyl)piperidine-1-carboxylate was prepared from tert-butyl 4-(4-bromo-3-fluorophenyl)piperidine-1-carboxylate (1.00 g, 2.79 mmol) and bis(pinacolato)diboron (1.00 g, 3.94 mmol), following a similar procedure to that described for the synthesis of tert-butyl 3-(3-fluoro-4-(4,4,5,5-tetramethyl-1,3,2-dioxaborolan-2-yl)phenyl)pyrrolidine-1-carboxylate and was isolated as a brown oil. The crude product was used directly in the next step without further purification.

Yield 1.00 g (crude). ¹H NMR not run. m/z: [ESI⁺] 350 (M+H−56)⁺.

### Synthesis of tert-butyl 4-(3-fluoro-4-(7-((3-(4-fluoropiperidin-1-yl)propyl)carbamoyl)-6-methoxybenzo[d]imidazo[2,1-b]thiazol-2-yl)phenyl)piperidine-1-carboxylate (6, Scheme 78)

Compound tert-butyl 4-(3-fluoro-4-(7-((3-(4-fluoropiperidin-1-yl)propyl)carbamoyl)-6-methoxybenzo[d]imidazo[2,1-b]thiazol-2-yl)phenyl)piperidine-1-carboxylate was prepared from 2-bromo-N-(3-(4-fluoropiperidin-1-yl)propyl)-6-methoxybenzo[d]imidazo[2,1-b]thiazole-7-carboxamide (300 mg, 0.639 mmol) and tert-butyl 4-(3-fluoro-4-(4,4,5,5-tetramethyl-1,3,2-dioxaborolan-2-yl)phenyl)piperidine-1-carboxylate (300 mg, crude) at 90° C., following a similar procedure to that described for the synthesis of tert-butyl 4-(4-bromo-3-fluorophenyl)-3,6-dihydropyridine-1(2H)-carboxylate and was isolated as a brown solid.

Yield 200 mg (47%). ¹H NMR (400 MHz, DMSO) δ 8.72 (d, J=3.6 Hz, 1H), 8.32 (s, 1H), 8.26 (t, J 5.6 Hz, 1H), 8.11-8.05 (m, 2H), 7.27-7.17 (m, 2H), 4.78-4.55 (m, 1H), 4.13-4.05 (m, 2H), 4.02 (s, 3H), 3.36-3.29 (m, 2H), 2.88-2.70 (m, 3H), 2.60-2.51 (m, 2H), 2.40-2.33 (m, 2H), 2.33-2.23 (m, 4H), 1.92-1.76 (m, 2H), 1.74-1.63 (m, 4H), 1.59-1.47 (m, 2H), 1.43 (s, 9H). m/z: [ESI⁺] 668 (M+H)⁺.

### Synthesis of tert-butyl 2-(4-(6-chloro-7-((3-(4-fluoropiperidin-1-yl)propyl)carbamoyl)benzo[d]imidazo[2,1-b]thiazol-2-yl)-3-fluorophenyl)pyrrolidine-1-carboxylate

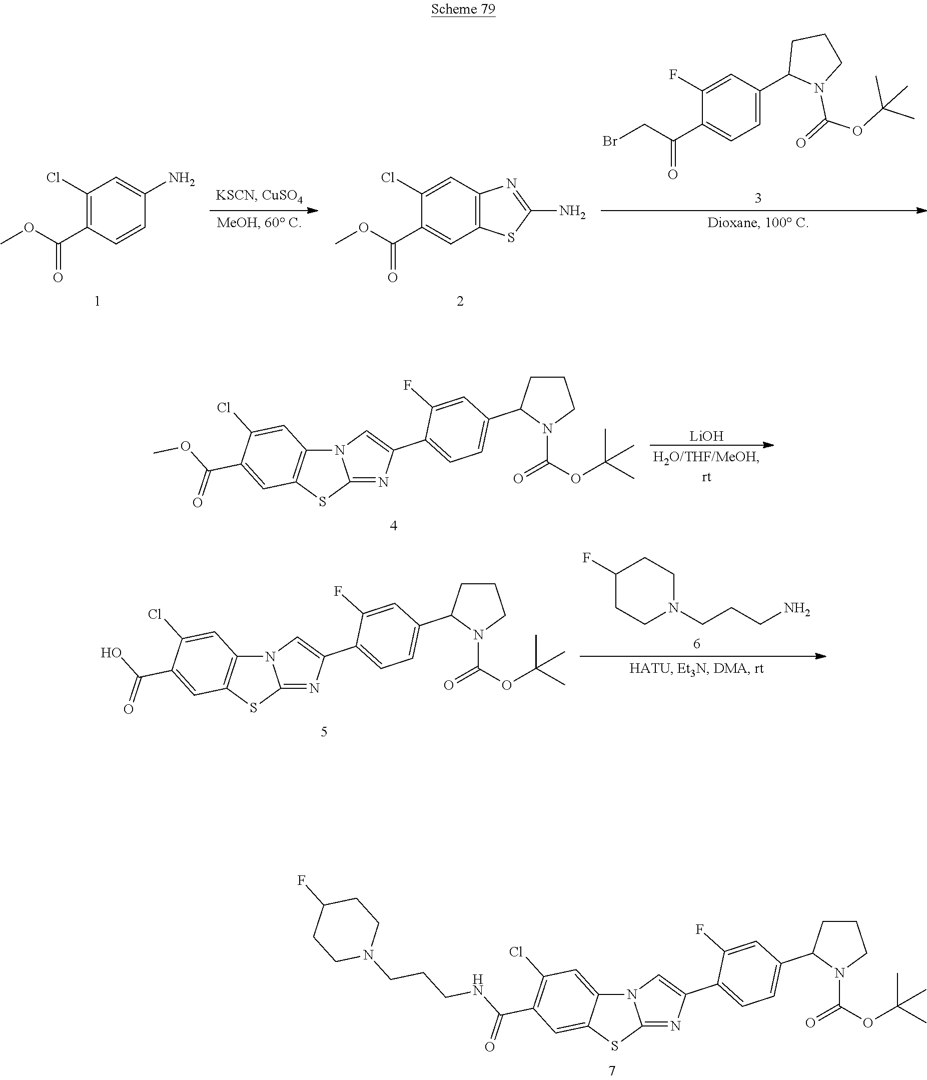

### Synthesis of methyl 2-amino-5-chlorobenzo[d]thiazole-6-carboxylate (2, Scheme 79)

Compound methyl 2-amino-5-chlorobenzo[d]thiazole-6-carboxylate was prepared from methyl 4-amino-2-chlorobenzoate (8.00 g, 43.10 mmol) and potassium thiocyanate (44.00 g, 452.77 mmol) following a similar procedure to that described for the synthesis of 6-bromo-5-(2-methoxyethoxy)benzo[d]thiazol-2-amine and was isolated as a purple solid.

Yield 4.00 g (38%). ¹H NMR (300 MHz, DMSO) δ 8.23 (s, 1H), 8.05 (br s, 2H), 7.42 (s, 1H), 3.83 (s, 3H). m/z: [ESI⁺] 243, 245 (M+H)⁺.

### Synthesis of methyl 2-(4-(1-(tert-butoxycarbonyl)pyrrolidin-2-yl)-2-fluorophenyl)-6-chlorobenzo[d]imidazo[2,1-b]thiazole-7-carboxylate (4, Scheme 79)

Compound methyl 2-(4-(1-(tert-butoxycarbonyl)pyrrolidin-2-yl)-2-fluorophenyl)-6-chlorobenzo[d]imidazo[2,1-b]thiazole-7-carboxylate was prepared from methyl 2-amino-5-chlorobenzo[d]thiazole-6-carboxylate (1.50 g, 6.18 mmol) and tert-butyl 2-(4-(2-bromoacetyl)-3-fluorophenyl)pyrrolidine-1-carboxylate (3.00 g, 7.77 mmol) following a similar procedure to that described for the synthesis of tert-butyl (S)-2-(4-(7-bromo-9-methyl-9H-benzo[d]imidazo[1,2-a]imidazol-2-yl)-3-fluorophenyl)pyrrolidine-1-carboxylate and was isolated as a brown solid.

Yield 1.00 g (31%). ¹H NMR (400 MHz, DMSO) δ 8.74 (d, J=3.6 Hz, 1H), 8.60 (s, 1H), 8.56 (s, 1H), 8.12-8.04 (m, 1H), 7.17-7.09 (m, 2H), 4.89-4.67 (m, 1H), 3.91 (s, 3H), 3.64-3.43 (m, 2H), 2.39-2.21 (m, 1H), 1.93-1.66 (m, 3H), 1.46-1.12 (m, 9H). m/z: [ESI⁺] 530, 532 (M+H)⁺.

### Synthesis of 2-(4-(1-(tert-butoxycarbonyl)pyrrolidin-2-yl)-2-fluorophenyl)-6-chlorobenzo[d]imidazo[2,1-b]thiazole-7-carboxylic Acid (5, Scheme 79)

Compound 2-(4-(1-(tert-butoxycarbonyl)pyrrolidin-2-yl)-2-fluorophenyl)-6-chlorobenzo[d]imidazo[2,1-b]thiazole-7-carboxylic acid was prepared from methyl 2-(4-(1-(tert-butoxycarbonyl)pyrrolidin-2-yl)-2-fluorophenyl)-6-chlorobenzo[d]imidazo[2,1-b]thiazole-7-carboxylate (1.00 g, 1.89 mmol) at room temperature, following a similar procedure to that described for the synthesis of 2-(1-(tert-butoxycarbonyl)-6-fluoroindolin-5-yl)benzo[d]imidazo[2,1-b]thiazole-7-carboxylic acid and was isolated as a red solid.

Yield 0.90 g (92%). ¹H NMR (400 MHz, DMSO) δ 8.73 (d, J=3.6 Hz, 1H), 8.56 (s, 1H), 8.51 (s, 1H), 8.13-8.00 (m, 1H), 7.19-7.07 (m, 2H), 4.90-4.71 (m, 1H), 3.65-3.51 (m, 2H), 2.40-2.18 (m, 1H), 1.94-1.66 (m, 3H), 1.45-1.08 (m, 9H). OH proton not observed. m/z: [ESI⁺] 516, 518 (M+H)⁺.

### Synthesis of tert-butyl 2-(4-(6-chloro-7-((3-(4-fluoropiperidin-1-yl)propyl)carbamoyl)benzo[d]imidazo[2,1-b]thiazol-2-yl)-3-fluorophenyl)pyrrolidine-1-carboxylate (7, Scheme 79)

Compound tert-butyl 2-(4-(6-chloro-7-((3-(4-fluoropiperidin-1-yl)propyl)carbamoyl)benzo[d]imidazo[2,1-b]thiazol-2-yl)-3-fluorophenyl)pyrrolidine-1-carboxylate was prepared from 2-(4-(1-(tert-butoxycarbonyl)pyrrolidin-2-yl)-2-fluorophenyl)-6-chlorobenzo[d]imidazo[2,1-b]thiazole-7-carboxylic acid (0.30 g, 0.58 mmol) and 3-(4-fluoropiperidin-1-yl)propan-1-amine (0.15 g, 0.94 mmol) following a similar procedure to that described for the synthesis of tert-butyl 6-fluoro-5-(7-((3-(4-fluoropiperidin-1-yl)propyl)carbamoyl)benzo[d]imidazo[2,1-b]thiazol-2-yl)indoline-1-carboxylate and was isolated as an off-white solid.

Yield 0.30 g (78%). ¹H NMR (300 MHz, DMSO) δ 8.73 (d, J=3.6 Hz, 1H), 8.62 (t, J=5.7 Hz, 1H), 8.50 (s, 1H), 8.18 (s, 1H), 8.13-8.03 (m, 1H), 7.18-7.08 (m, 2H), 4.92-4.63 (m, 2H), 3.64-3.41 (m, 2H), 3.36-3.27 (m, 2H), 2.90-2.60 (m, 6H), 2.41-2.22 (m, 1H), 1.91-1.72 (m, 9H), 1.45-1.10 (m, 9H). m/z: [ESI⁺] 658, 660 (M+H)⁺.

### Synthesis of tert-butyl (R)-2-(3-fluoro-4-(6-methoxy-7-((1-methylpiperidin-4-yl)carbamoyl)benzo[d]imidazo[2,1-b]thiazol-2-yl)phenyl)pyrrolidine-1-carboxylate and tert-butyl (S)-2-(3-fluoro-4-(6-methoxy-7-((1-methylpiperidin-4-yl)carbamoyl)benzo[d]imidazo[2,1-b]thiazol-2-yl)phenyl)pyrrolidine-1-carboxylate

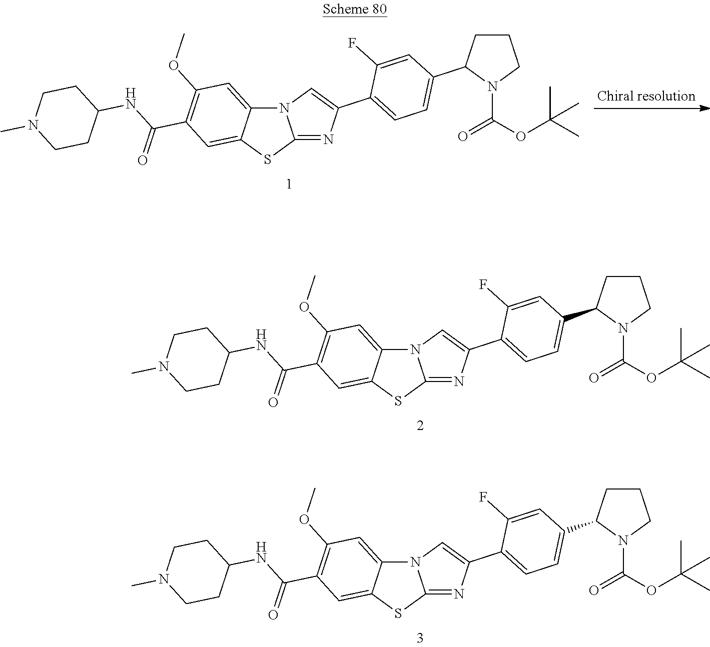

Tert-butyl 2-(3-fluoro-4-(6-methoxy-7-((1-methylpiperidin-4-yl)carbamoyl)benzo[d]imidazo[2,1-b]thiazol-2-yl)phenyl)pyrrolidine-1-carboxylate (450 mg, 0.740 mmol) was separated by Prep-CHIRAL-HPLC using the following conditions; Column: CHIRAL ART Cellulose-SC, 2×25 cm, 5 μm; Mobile Phase A: hexane (plus 0.5% 2 N ammonia-methanol), Mobile Phase B: ethanol:dichloromethane=1:1; Flow rate: 20 mL/min; Gradient: 35% B in 24 min; UV detector: 220/254 nm. The faster eluting peak at 16.33 min was collected and concentrated under reduced pressure to afford tert-butyl (R)-2-(3-fluoro-4-(6-methoxy-7-((1-methylpiperidin-4-yl)carbamoyl)benzo[d]imidazo[2,1-b]thiazol-2-yl)phenyl)pyrrolidine-1-carboxylate as a yellow solid.

Yield 100 mg (22%). ¹H NMR (400 MHz, CD₃OD) δ 8.55 (d, J=3.6 Hz, 1H), 8.30 (s, 1H), 8.12-8.01 (m, 1H), 7.86 (s, 1H), 7.18-7.11 (m, 1H), 7.11-7.04 (m, 1H), 5.00-4.85 (m, 1H), 4.13 (s, 3H), 4.13-4.02 (m, 1H), 3.72-3.54 (m, 2H), 3.25-3.12 (m, 2H), 2.80-2.66 (m, 2H), 2.62 (s, 3H), 2.49-2.31 (m, 1H), 2.23-2.11 (m, 2H), 2.01-1.72 (m, 5H), 1.53-1.22 (m, 9H). NH proton not observed. m/z: [ESI⁺] 608 (M+H)⁺. [α]²⁵_{D}=+38° (c=1 mg/mL, methanol).

The slower eluting peak at 19.85 min was collected and concentrated under reduced pressure to afford tert-butyl (S)-2-(3-fluoro-4-(6-methoxy-7-((1-methylpiperidin-4-yl)carbamoyl)benzo[d]imidazo[2,1-b]thiazol-2-yl)phenyl)pyrrolidine-1-carboxylate as a yellow solid.

Yield 110 mg (24%). ¹H NMR (400 MHz, CD₃OD) δ 8.55 (d, J=3.6 Hz, 1H), 8.30 (s, 1H), 8.12-8.01 (m, 1H), 7.86 (s, 1H), 7.18-7.11 (m, 1H), 7.11-7.04 (m, 1H), 5.00-4.85 (m, 1H), 4.13 (s, 3H), 4.13-4.02 (m, 1H), 3.72-3.54 (m, 2H), 3.25-3.12 (m, 2H), 2.80-2.66 (m, 2H), 2.62 (s, 3H), 2.49-2.31 (m, 1H), 2.23-2.11 (m, 2H), 2.01-1.72 (m, 5H), 1.53-1.22 (m, 9H). NH proton not observed. m/z: [ESI⁺] 608 (M+H)⁺. [α]²⁵_{D}=−42° (c=1 mg/mL, methanol).

### Synthesis of tert-butyl 4-(3-fluoro-4-(7-((3-(4-fluoropiperidin-1-yl)propyl)carbamoyl)-6-methoxybenzo[d]imidazo[2,1-b]thiazol-2-yl)phenyl)-4-hydroxypiperidine-1-carboxylate

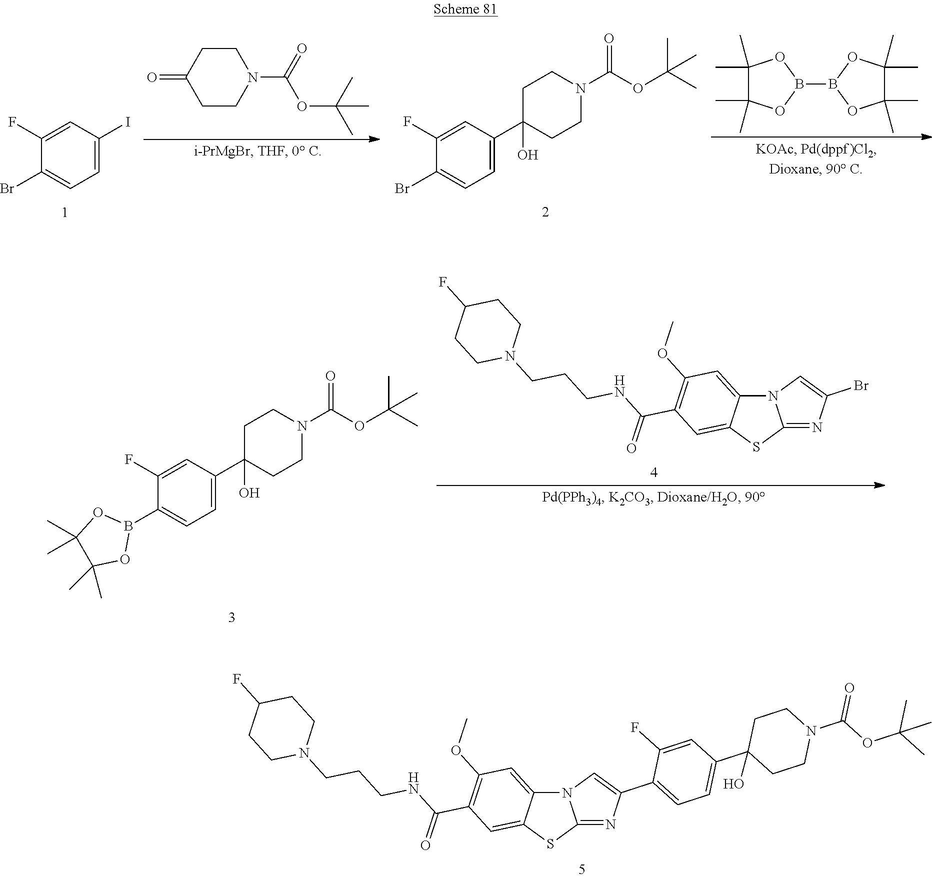

### Synthesis of tert-butyl 4-(4-bromo-3-fluorophenyl)-4-hydroxypiperidine-1-carboxylate (2, Scheme 81)

Compound tert-butyl 4-(4-bromo-3-fluorophenyl)-4-hydroxypiperidine-1-carboxylate was prepared from 1-bromo-2-fluoro-4-iodobenzene (4.53 g, 15.05 mmol) and tert-butyl 4-oxopiperidine-1-carboxylate (3.00 g, 15.06 mmol) following a similar procedure to that described for the synthesis of tert-butyl (4-(4-bromo-2,3-difluorophenyl)-4-oxobutyl)carbamate and was isolated as a colorless oil.

Yield 3.51 g (62%). ¹H NMR (400 MHz, CDCl₃) δ 7.55 (dd, J=7.2, 8.4 Hz, 1H), 7.30 (dd, J=2.0 10.4 Hz, 1H), 7.14 (dd, J=2.0, 8.4 Hz, 1H), 4.12-4.03 (m, 2H), 3.26-3.16 (m, 2H), 2.02-1.90 (m, 2H), 1.75-1.66 (m, 2H), 1.51 (s, 9H). OH proton not observed. m/z: [ESI⁻] 372, 374 (M−H)⁻.

### Synthesis of tert-butyl 4-(3-fluoro-4-(4,4,5,5-tetramethyl-1,3,2-dioxaborolan-2-yl)phenyl)-4-hydroxypiperidine-1-carboxylate (3, Scheme 81)

Compound tert-butyl 4-(3-fluoro-4-(4,4,5,5-tetramethyl-1,3,2-dioxaborolan-2-yl)phenyl)-4-hydroxypiperidine-1-carboxylate was prepared from tert-butyl 4-(4-bromo-3-fluorophenyl)-4-hydroxypiperidine-1-carboxylate (300 mg, 0.802 mmol) and bis(pinacolato)diboron (300 mg, 1.181 mmol), following a similar procedure to that described for the synthesis of tert-butyl 3-(3-fluoro-4-(4,4,5,5-tetramethyl-1,3,2-dioxaborolan-2-yl)phenyl)pyrrolidine-1-carboxylate and was isolated as a brown solid. The compound was used directly in the next step without further purification.

Yield 340 mg (crude). ¹H NMR not run. m/z: [ESI⁻] 420 (M−H)⁻.

### Synthesis of tert-butyl 4-(3-fluoro-4-(7-((3-(4-fluoropiperidin-1-yl)propyl)carbamoyl)-6-methoxybenzo[d]imidazo[2,1-b]thiazol-2-yl)phenyl)-4-hydroxypiperidine-1-carboxylate (5, Scheme 81)

Compound tert-butyl 4-(3-fluoro-4-(7-((3-(4-fluoropiperidin-1-yl)propyl)carbamoyl)-6-methoxybenzo[d]imidazo[2,1-b]thiazol-2-yl)phenyl)-4-hydroxypiperidine-1-carboxylate was prepared from 2-bromo-N-(3-(4-fluoropiperidin-1-yl)propyl)-6-methoxybenzo[d]imidazo[2,1-b]thiazole-7-carboxamide (200 mg, 0.426 mmol) and tert-butyl 4-(3-fluoro-4-(4,4,5,5-tetramethyl-1,3,2-dioxaborolan-2-yl)phenyl)-4-hydroxypiperidine-1-carboxylate (340 mg, crude) at 90° C., following a similar procedure to that described for the synthesis of tert-butyl 4-(4-bromo-3-fluorophenyl)-3,6-dihydropyridine-1(2H)-carboxylate and was isolated as a brown solid.

Yield 135 mg (46%). ¹H NMR (300 MHz, CDCl₃) δ 8.38 (s, 1H), 8.14-8.05 (m, 1H), 8.00 (d, J=3.6 Hz, 1H), 7.30-7.28 (m, 1H), 7.26-7.23 (m, 1H), 7.16-7.13 (m, 1H), 4.86-4.60 (m, 1H), 4.19-3.98 (m, 5H), 3.62-3.53 (m, 2H), 3.38-3.23 (m, 2H), 2.76-2.63 (m, 2H), 2.63-2.42 (m, 4H), 2.12-1.83 (m, 8H), 1.83-1.72 (m, 2H), 1.52 (s, 9H). OH and NH protons not observed. m/z: [ESI⁺] 684 (M+H)⁺.

### Synthesis of tert-butyl 3-(3-fluoro-4-(7-((3-(4-fluoropiperidin-1-yl)propyl)carbamoyl)-6-methoxybenzo[d]imidazo[2,1-b]thiazol-2-yl)phenyl)piperidine-1-carboxylate

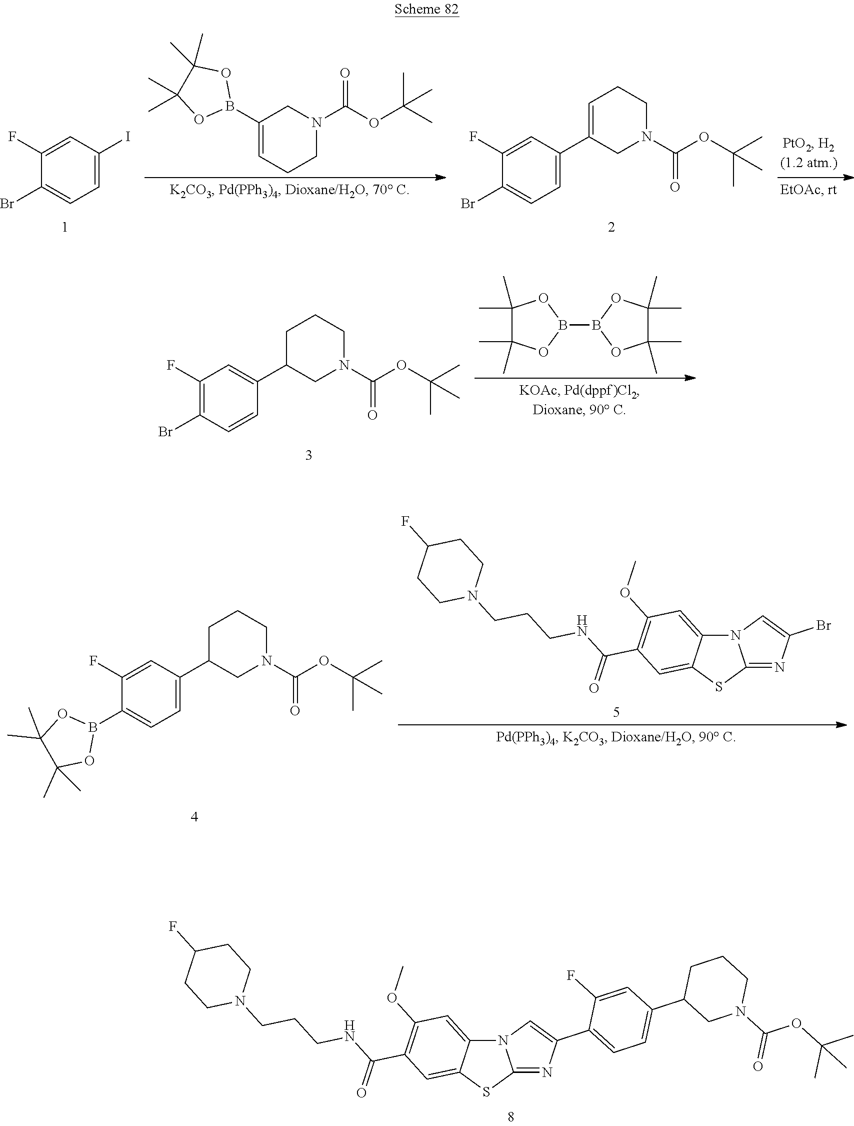

### Synthesis of tert-butyl 5-(4-bromo-3-fluorophenyl)-3,6-dihydropyridine-1(2H)-carboxylate (2, Scheme 82)

Compound tert-butyl 5-(4-bromo-3-fluorophenyl)-3,6-dihydropyridine-1(2H)-carboxylate was prepared from 1-bromo-2-fluoro-4-iodobenzene (3.30 g, 10.97 mmol) and tert-butyl 5-(4,4,5,5-tetramethyl-1,3,2-dioxaborolan-2-yl)-3,6-dihydropyridine-1(2H)-carboxylate (4.00 g, 12.94 mmol) following a similar procedure to that described for the synthesis of tert-butyl 4-(4-bromo-3-fluorophenyl)-3,6-dihydropyridine-1(2H)-carboxylate and was isolated as an off-white solid.

Yield 3.00 g (77%). ¹H NMR (400 MHz, DMSO) δ 7.68 (dd, J=7.2, 8.4 Hz, 1H), 7.44 (dd, J=2.0, 10.4 Hz, 1H), 7.21 (dd, J=2.0, 8.4 Hz, 1H), 6.48-6.40 (m, 1H), 4.23-4.16 (m, 2H), 3.50-3.42 (m, 2H), 2.31-2.21 (m, 2H), 1.43 (s, 9H). m/z: [ESI⁺] 300, 302 (M+H−56)⁺.

### Synthesis of tert-butyl 3-(4-bromo-3-fluorophenyl)piperidine-1-carboxylate (3, Scheme 82)

Compound tert-butyl 3-(4-bromo-3-fluorophenyl)piperidine-1-carboxylate was prepared from tert-butyl 5-(4-bromo-3-fluorophenyl)-3,6-dihydropyridine-1(2H)-carboxylate (1.90 g, 5.33 mmol) following a similar procedure to that described for the synthesis of tert-butyl 4-(4-bromo-3-fluorophenyl)piperidine-1-carboxylate and was isolated as a colorless oil.

Yield 0.61 g (32%). ¹H NMR (300 MHz, DMSO) δ 7.69-7.59 (m, 1H), 7.38-7.29 (m, 1H), 7.16-7.06 (m, 1H), 4.02-3.86 (m, 2H), 3.75-3.52 (m, 2H), 3.00-2.57 (m, 3H), 1.76-1.57 (m, 2H), 1.41 (s, 9H). m/z: [ESI⁺] 302, 304 (M+H−56)⁺.

### Synthesis of tert-butyl 3-(3-fluoro-4-(4,4,5,5-tetramethyl-1,3,2-dioxaborolan-2-yl)phenyl)piperidine-1-carboxylate (4, Scheme 82)

Compound tert-butyl 3-(3-fluoro-4-(4,4,5,5-tetramethyl-1,3,2-dioxaborolan-2-yl)phenyl)piperidine-1-carboxylate was prepared from tert-butyl 3-(4-bromo-3-fluorophenyl)piperidine-1-carboxylate (200 mg, 0.558 mmol) and bis(pinacolato)diboron (213 mg, 0.839 mmol) following a similar procedure to that described for the synthesis of tert-butyl 3-(3-fluoro-4-(4,4,5,5-tetramethyl-1,3,2-dioxaborolan-2-yl)phenyl)pyrrolidine-1-carboxylate and was isolated as a brown solid. The compound was used directly in the next step without further purification.

Yield 200 mg (crude). ¹H NMR not run. m/z: [ESI⁺] 350 (M+H−56)⁺.

### Synthesis of tert-butyl 3-(3-fluoro-4-(7-((3-(4-fluoropiperidin-1-yl)propyl)carbamoyl)-6-methoxybenzo[d]imidazo[2,1-b]thiazol-2-yl)phenyl)piperidine-1-carboxylate (6, Scheme 82)

Compound tert-butyl 3-(3-fluoro-4-(7-((3-(4-fluoropiperidin-1-yl)propyl)carbamoyl)-6-methoxybenzo[d]imidazo[2,1-b]thiazol-2-yl)phenyl)piperidine-1-carboxylate was prepared from 2-bromo-N-(3-(4-fluoropiperidin-1-yl)propyl)-6-methoxybenzo[d]imidazo[2,1-b]thiazole-7-carboxamide (200 mg, 0.426 mmol) and tert-butyl 3-(3-fluoro-4-(4,4,5,5-tetramethyl-1,3,2-dioxaborolan-2-yl)phenyl)piperidine-1-carboxylate (200 mg, crude) at 90° C., following a similar procedure to that described for the synthesis of tert-butyl 4-(4-bromo-3-fluorophenyl)-3,6-dihydropyridine-1(2H)-carboxylate and was isolated as a yellow solid.

Yield 80 mg (28%). ¹H NMR (400 MHz, DMSO) δ 8.73 (d, J=3.6 Hz, 1H), 8.32 (s, 1H), 8.26 (t, J 5.6 Hz, 1H), 8.13-8.05 (m, 2H), 7.31-7.25 (m, 1H), 7.25-7.20 (m, 1H), 4.77-4.56 (m, 1H), 4.02 (s, 3H), 3.99-3.92 (m, 1H), 3.35-3.23 (m, 2H), 2.88-2.75 (m, 1H), 2.75-2.62 (m, 1H), 2.59-2.42 (m, 2H), 2.40-2.21 (m, 6H), 1.97-1.77 (m, 2H), 1.77-1.56 (m, 8H), 1.43 (s, 9H). m/z: [ESI⁺] 668 (M+H)⁺.

### Synthesis of tert-butyl 2-(3-fluoro-4-(7-(1-methylpiperidine-4-carboxamido)benzo[d]imidazo[2,1-b]thiazol-2-yl)phenyl)pyrrolidine-1-carboxylate

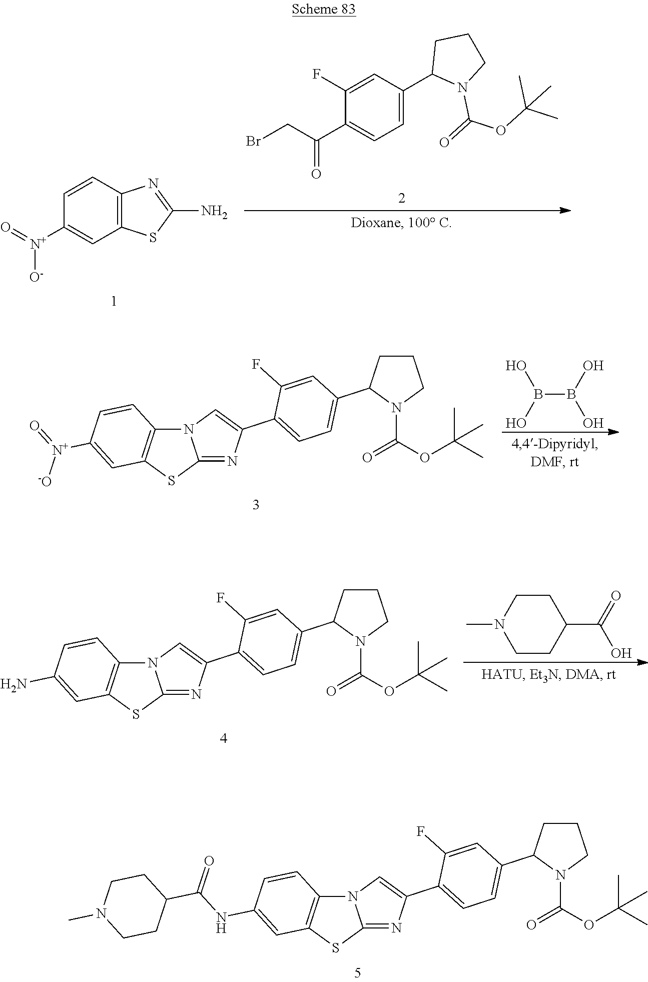

### Synthesis of tert-butyl 2-(3-fluoro-4-(7-nitrobenzo[d]imidazo[2,1-b]thiazol-2-yl)phenyl)pyrrolidine-1-carboxylate ((3, Scheme 83)

Compound tert-butyl 2-(3-fluoro-4-(7-nitrobenzo[d]imidazo[2,1-b]thiazol-2-yl)phenyl)pyrrolidine-1-carboxylate was prepared from 6-nitrobenzo[d]thiazol-2-amine (1.00 g, 5.12 mmol) and tert-butyl 2-(4-(2-bromoacetyl)-3-fluorophenyl)pyrrolidine-1-carboxylate (2.18 g, 5.64 mmol) following a similar procedure to that described for the synthesis of tert-butyl (S)-2-(4-(7-bromo-9-methyl-9H-benzo[d]imidazo[1,2-a]imidazol-2-yl)-3-fluorophenyl)pyrrolidine-1-carboxylate and was isolated as an orange solid.

Yield 0.90 g (36%). ¹H NMR (400 MHz, CD₃OD) δ 8.93 (d, J=2.0 Hz, 1H), 8.54 (s, 1H), 8.46 (dd, J 2.0, 8.8 Hz, 1H), 8.16 (d, J=8.8 Hz, 1H), 8.12-8.05 (m, 1H), 7.17-7.13 (m, 1H), 7.10-7.04 (m, 1H), 4.97-4.76 (m, 1H), 3.70-3.55 (m, 2H), 2.49-2.31 (m, 1H), 2.06-1.79 (m, 3H), 1.52-1.18 (m, 9H). m/z: [ESI⁺] 483 (M+H)⁺.

### Synthesis of tert-butyl 2-(4-(7-aminobenzo[d]imidazo[2,1-b]thiazol-2-yl)-3-fluorophenyl)pyrrolidine-1-carboxylate (4, Scheme 83)

To a stirred solution of tert-butyl 2-(3-fluoro-4-(7-nitrobenzo[d]imidazo[2,1-b]thiazol-2-yl)phenyl)pyrrolidine-1-carboxylate (900 mg, 1.865 mmol) in N,N-dimethylformamide (9 mL) were added hypodiboric acid (334 mg, 3.726 mmol) and 4,4′-bipyridine (58 mg, 0.371 mmol) sequentially as single portions at room temperature. The reaction mixture was stirred for an additional 30 min. The reaction solution was purified directly by reverse phase flash chromatography using the following conditions; Column: Spherical C18, 20-40 μm, 330 g; Mobile Phase A: water (plus 10 mM ammonium bicarbonate); Mobile Phase B: acetonitrile; Flow rate: 60 mL/min; Gradient: 60%-80% B in 20 min; UV Detector: 254 nm. The fractions containing desired product were collected at 76% B and concentrated under reduced pressure to afford tert-butyl 2-(4-(7-aminobenzo[d]imidazo[2,1-b]thiazol-2-yl)-3-fluorophenyl)pyrrolidine-1-carboxylate as an orange solid.

Yield 450 mg (53%). ¹H NMR (400 MHz, CDCl₃) δ 8.20-8.12 (m, 1H), 8.05 (s, 1H), 7.48-7.38 (m, 1H), 7.11-7.05 (m, 1H), 7.02-6.94 (m, 2H), 6.82-6.73 (m, 1H), 5.04-4.75 (m, 1H), 3.96 (br s, 2H), 3.73-3.54 (m, 2H), 2.47-2.25 (m, 1H), 2.04-1.82 (m, 3H), 1.54-1.21 (m, 9H). m/z: [ESI⁺] 453 (M+H)⁺.

### Synthesis of tert-butyl 2-(3-fluoro-4-(7-(1-methylpiperidine-4-carboxamido)benzo[d]imidazo[2,1-b]thiazol-2-yl)phenyl)pyrrolidine-1-carboxylate (5, Scheme 83)

Compound tert-butyl 2-(3-fluoro-4-(7-(1-methylpiperidine-4-carboxamido)benzo[d]imidazo[2,1-b]thiazol-2-yl)phenyl)pyrrolidine-1-carboxylate was prepared from tert-butyl 2-(4-(7-aminobenzo[d]imidazo[2,1-b]thiazol-2-yl)-3-fluorophenyl)pyrrolidine-1-carboxylate (100 mg, 0.221 mmol) and 1-methylpiperidine-4-carboxylic acid (63 mg, 0.440 mmol) following a similar procedure to that described for the synthesis of tert-butyl 6-fluoro-5-(7-((3-(4-fluoropiperidin-1-yl)propyl)carbamoyl)benzo[d]imidazo[2,1-b]thiazol-2-yl)indoline-1-carboxylate and was isolated as an off-white solid.

Yield 105 mg (82%). ¹H NMR (400 MHz, CDCl₃) δ 8.68 (br s, 1H), 8.27-8.06 (m, 1H), 7.92-7.78 (m, 1H), 7.73-7.62 (m, 1H), 7.62-7.52 (m, 1H), 7.27-7.12 (m, 1H), 6.94-6.85 (m, 1H), 6.85-6.72 (m, 1H), 4.94-4.76 (m, 1H), 3.74-3.53 (m, 2H), 3.08-2.90 (m, 2H), 2.47-2.25 (m, 5H), 2.16-1.77 (m, 9H), 1.61-1.27 (m, 9H). m/z: [ESI⁺] 578 (M+H)⁺.

### Synthesis of tert-butyl 2-(3-fluoro-4-(7-(tetrahydro-2H-pyran-4-carboxamido)benzo[d]imidazo[2,1-b]thiazol-2-yl)phenyl)pyrrolidine-1-carboxylate

Compound tert-butyl 2-(3-fluoro-4-(7-(tetrahydro-2H-pyran-4-carboxamido)benzo[d]imidazo[2,1-b]thiazol-2-yl)phenyl)pyrrolidine-1-carboxylate was prepared from tert-butyl 2-(4-(7-aminobenzo[d]imidazo[2,1-b]thiazol-2-yl)-3-fluorophenyl)pyrrolidine-1-carboxylate (100 mg, 0.221 mmol) and tetrahydro-2H-pyran-4-carboxylic acid (58 mg, 0.446 mmol) following a similar procedure to that described for the synthesis of tert-butyl 6-fluoro-5-(7-((3-(4-fluoropiperidin-1-yl)propyl)carbamoyl)benzo[d]imidazo[2,1-b]thiazol-2-yl)indoline-1-carboxylate and was isolated as an off-white solid.

Yield 103 mg (83%). ¹H NMR (400 MHz, CDCl₃) δ 9.10 (br s, 1H), 8.31-8.04 (m, 1H), 7.87-7.75 (m, 1H), 7.75-7.66 (m, 1H), 7.66-7.54 (m, 1H), 7.28-7.17 (m, 1H), 6.92-6.81 (m, 1H), 6.81-6.70 (m, 1H), 4.93-4.74 (m, 1H), 4.17-4.02 (m, 2H), 3.76-3.57 (m, 2H), 3.56-3.41 (m, 2H), 2.71-2.52 (m, 1H), 2.42-2.26 (m, 1H), 2.10-1.74 (m, 7H), 1.67-1.24 (m, 9H). m/z: [ESI⁺] 565 (M+H)⁺.

### Final Compounds

### Synthesis of 2-(2,3-difluoro-4-(pyrrolidin-2-yl)phenyl)-N-(3-(4-fluoropiperidin-1-yl)propyl)-6-methoxybenzo[d]imidazo[2,1-b]thiazole-7-carboxamide Dihydrochloride (Compound 187)

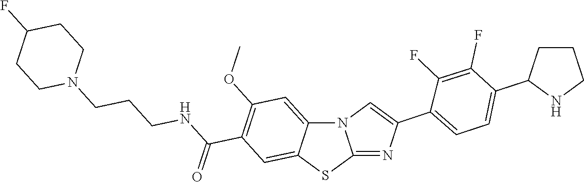 [SMILES: COc1cc2c(cc1C(=O)NCCCN1CCC(F)CC1)sc1nc(-c3ccc(C4CCCN4)c(F)c3F)cn12]

Compound 2-(2,3-difluoro-4-(pyrrolidin-2-yl)phenyl)-N-(3-(4-fluoropiperidin-1-yl)propyl)-6-methoxybenzo[d]imidazo[2,1-b]thiazole-7-carboxamide dihydrochloride was prepared from tert-butyl 2-(2,3-difluoro-4-(7-((3-(4-fluoropiperidin-1-yl)propyl)carbamoyl)-6-methoxybenzo[d]imidazo[2,1-b]thiazol-2-yl)phenyl)pyrrolidine-1-carboxylate (100 mg, 0.149 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride and was isolated as an off-white solid.

Yield 20 mg (21%). ¹H NMR (400 MHz, DMSO) δ 10.65 (br s, 1H), 10.05 (br s, 1H), 9.21 (br s, 1H), 8.94 (d, J=3.6 Hz, 1H), 8.50 (t, J=5.6 Hz, 1H), 8.38 (s, 1H), 8.14 (s, 1H), 8.06-7.98 (m, 1H), 7.60-7.53 (m, 1H), 5.13-4.72 (m, 2H), 4.05 (s, 3H), 3.45-3.30 (m, 6H), 3.19-2.95 (m, 4H), 2.49-2.35 (m, 1H), 2.30-1.93 (m, 9H). ¹⁹F NMR (376 MHz, DMSO) δ −140.12, −140.17, −140.58, −140.64, −175.67, −186.62. m/z: [ESI⁺] 572 (M+H)⁺. (C₂₉H₃₄Cl₂F₃N₅O₂S).

### Synthesis of 2-(2-fluoro-4-(piperidin-4-yl)phenyl)-N-(3-(4-fluoropiperidin-1-yl)propyl)-6-methoxybenzo[d]imidazo[2,1-b]thiazole-7-carboxamide Dihydrochloride (Compound 186)

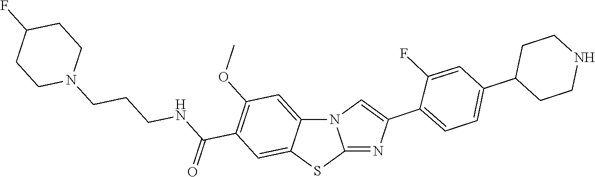 [SMILES: COc1cc2c(cc1C(=O)NCCCN1CCC(F)CC1)sc1nc(-c3ccc(C4CCNCC4)cc3F)cn12]

Compound 2-(2-fluoro-4-(piperidin-4-yl)phenyl)-N-(3-(4-fluoropiperidin-1-yl)propyl)-6-methoxybenzo[d]imidazo[2,1-b]thiazole-7-carboxamide dihydrochloride was prepared from tert-butyl 4-(3-fluoro-4-(7-((3-(4-fluoropiperidin-1-yl)propyl)carbamoyl)-6-methoxybenzo[d]imidazo[2,1-b]thiazol-2-yl)phenyl)piperidine-1-carboxylate (100 mg, 0.150 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride and was isolated as an off-white solid.

Yield 15 mg (16%). ¹H NMR (400 MHz, DMSO) δ 10.69 (br s, 1H), 8.94 (br s, 2H), 8.76 (d, J=3.6 Hz, 1H), 8.50 (t, J=5.6 Hz, 1H), 8.36 (s, 1H), 8.17-8.06 (m, 2H), 7.27-7.13 (m, 2H), 5.11-4.72 (m, 1H), 4.04 (s, 3H), 3.55-3.28 (m, 6H), 3.17-2.84 (m, 7H), 2.28-1.77 (m, 10H). ¹⁹F NMR (376 MHz, DMSO) δ −113.60, −175.56, −186.61. m/z: [ESI⁺] 568 (M+H)⁺. (C₃₀H₃₇Cl₂F₂N₅O₂S).

### Synthesis of 2-(2-fluoro-4-(pyrrolidin-3-yl)phenyl)-6-methoxybenzo[d]imidazo[2,1-b]thiazole-7-carboxamide Hydrochloride (Compound 183)

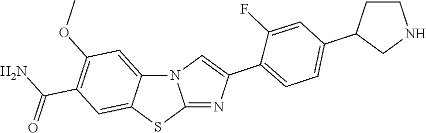 [SMILES: COc1cc2c(cc1C(N)=O)sc1nc(-c3ccc(C4CCNC4)cc3F)cn12]

Compound 2-(2-fluoro-4-(pyrrolidin-3-yl)phenyl)-6-methoxybenzo[d]imidazo[2,1-b]thiazole-7-carboxamide hydrochloride was prepared from tert-butyl 3-(4-(7-carbamoyl-6-methoxybenzo[d]imidazo[2,1-b]thiazol-2-yl)-3-fluorophenyl)pyrrolidine-1-carboxylate (70 mg, 0.137 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride and was isolated as a brown solid.

Yield 14 mg (23%). ¹H NMR (400 MHz, DMSO) δ 9.62 (br s, 2H), 8.79 (d, J=3.6 Hz, 1H), 8.42 (s, 1H), 8.15-8.07 (m, 2H), 7.74 (br s, 1H), 7.65 (br s, 1H), 7.47-7.38 (m, 1H), 7.36-7.25 (m, 1H), 4.03 (s, 3H), 3.68-3.57 (m, 1H), 3.57-3.46 (m, 1H), 3.46-3.34 (m, 1H), 3.27-3.17 (m, 1H), 3.17-3.04 (m, 1H), 2.45-2.34 (m, 1H), 2.05-1.92 (m, 1H). ¹⁹F NMR (376 MHz, DMSO) δ −113.32. m/z: [ESI⁺] 411 (M+H)⁺. (C₂₁H₂₀ClFN₄O₂S).

### Synthesis of 2-(2-fluoro-4-(pyrrolidin-3-yl)phenyl)-6-methoxy-N-methylbenzo[d]imidazo[2,1-b]thiazole-7-carboxamide Hydrochloride (Compound 184)

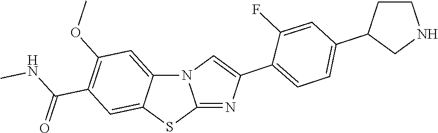 [SMILES: CNC(=O)c1cc2sc3nc(-c4ccc(C5CCNC5)cc4F)cn3c2cc1OC]

Compound 2-(2-fluoro-4-(pyrrolidin-3-yl)phenyl)-6-methoxy-N-methylbenzo[d]imidazo[2,1-b]thiazole-7-carboxamide hydrochloride was prepared from tert-butyl 3-(3-fluoro-4-(6-methoxy-7-(methylcarbamoyl)benzo[d]imidazo[2,1-b]thiazol-2-yl)phenyl)pyrrolidine-1-carboxylate (80 mg, 0.152 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride and was isolated as a brown solid.

Yield 20 mg (28%). ¹H NMR (400 MHz, DMSO) δ 9.68 (br s, 2H), 8.79 (d, J=3.6 Hz, 1H), 8.38 (s, 1H), 8.25 (q, J=4.8 Hz, 1H), 8.15-8.04 (m, 2H), 7.42 (dd, J=1.6, 12.8 Hz, 1H), 7.30 (dd, J=1.6, 8.0 Hz, 1H), 4.02 (s, 3H), 3.68-3.59 (m, 1H), 3.58-3.45 (m, 1H), 3.45-3.37 (m, 1H), 3.27-3.18 (m, 1H), 3.18-3.06 (m, 1H), 2.83 (d, J=4.8 Hz, 3H), 2.45-2.33 (m, 1H), 2.04-1.93 (m, 1H). ¹⁹F NMR (376 MHz, DMSO) δ −113.24. m/z: [ESI⁺] 425 (M+H)⁺. (C₂₂H₂₂ClFN₄O₂S).

### Synthesis of 2-(2-fluoro-4-(pyrrolidin-3-yl)phenyl)-6-methoxy-N-(1-methylpiperidin-4-yl)benzo[d]imidazo[2,1-b]thiazole-7-carboxamide Dihydrochloride (Compound 185)

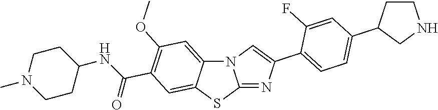 [SMILES: COc1cc2c(cc1C(=O)NC1CCN(C)CC1)sc1nc(-c3ccc(C4CCNC4)cc3F)cn12]

Compound 2-(2-fluoro-4-(pyrrolidin-3-yl)phenyl)-6-methoxy-N-(1-methylpiperidin-4-yl)benzo[d]imidazo[2,1-b]thiazole-7-carboxamide dihydrochloride was prepared from tert-butyl 3-(3-fluoro-4-(6-methoxy-7-((1-methylpiperidin-4-yl)carbamoyl)benzo[d]imidazo[2,1-b]thiazol-2-yl)phenyl)pyrrolidine-1-carboxylate (100 mg, 0.165 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride and was isolated as an off-white solid.

Yield 31 mg (32%). ¹H NMR (400 MHz, DMSO) δ 11.08 (br s, 1H), 9.86 (br s, 2H), 8.82 (d, J=3.6 Hz, 1H), 8.42-8.28 (m, 1H), 8.23 (s, 1H), 8.16-8.05 (m, 2H), 7.46-7.36 (m, 1H), 7.34-7.25 (m, 1H), 3.99 (s, 3H), 3.69-3.58 (m, 1H), 3.58-3.46 (m, 1H), 3.46-3.35 (m, 3H), 3.35-3.04 (m, 4H), 2.73 (s, 3H), 2.44-2.34 (m, 1H), 2.18-1.84 (m, 6H). ¹⁹F NMR (376 MHz, DMSO) δ −113.01. m/z: [ESI⁺] 508 (M+H)⁺. (C₂₇H₃₂Cl₂FN₅O₂S).

### Synthesis of 2-(2-fluoro-4-(pyrrolidin-3-yl)phenyl)-N-(3-(4-fluoropiperidin-1-yl)propyl)-6-methoxybenzo[d]imidazo[2,1-b]thiazole-7-carboxamide dihydrochloride (Compound 182)

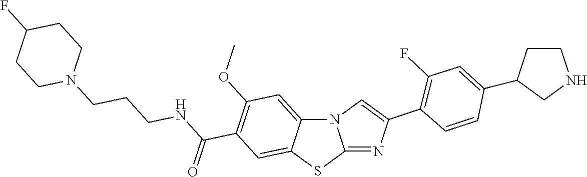 [SMILES: COc1cc2c(cc1C(=O)NCCCN1CCC(F)CC1)sc1nc(-c3ccc(C4CCNC4)cc3F)cn12]

Compound 2-(2-fluoro-4-(pyrrolidin-3-yl)phenyl)-N-(3-(4-fluoropiperidin-1-yl)propyl)-6-methoxybenzo[d]imidazo[2,1-b]thiazole-7-carboxamide dihydrochloride was prepared from tert-butyl 3-(3-fluoro-4-(7-((3-(4-fluoropiperidin-1-yl)propyl)carbamoyl)-6-methoxybenzo[d]imidazo[2,1-b]thiazol-2-yl)phenyl)pyrrolidine-1-carboxylate (80 mg, 0.122 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride and was isolated as an off-white solid.

Yield 12 mg (16%). ¹H NMR (400 MHz, DMSO) δ 10.97 (br s, 1H), 9.57 (br s, 2H), 8.80 (d, J=3.6 Hz, 1H), 8.50 (t, J=6.4 Hz, 1H), 8.37 (s, 1H), 8.18-8.07 (m, 2H), 7.45-7.39 (m, 1H), 7.36-7.25 (m, 1H), 5.12-4.70 (m, 1H), 4.05 (s, 3H), 3.70-3.58 (m, 1H), 3.58-3.46 (m, 2H), 3.46-3.32 (m, 4H), 3.30-2.95 (m, 5H), 2.45-2.34 (m, 1H), 2.34-1.93 (m, 8H). 19F NMR (376 MHz, DMSO) δ −113.38, −175.83, −186.63. m/z: [ESI⁺] 554 (M+H)⁺. (C₂₉H₃₅Cl₂F₂N₅O₂S).

### Synthesis of 6-chloro-2-(2-fluoro-4-(pyrrolidin-2-yl)phenyl)-N-(3-(4-fluoropiperidin-1-yl)propyl)benzo[d]imidazo[2,1-b]thiazole-7-carboxamide Dihydrochloride (Compound 188)

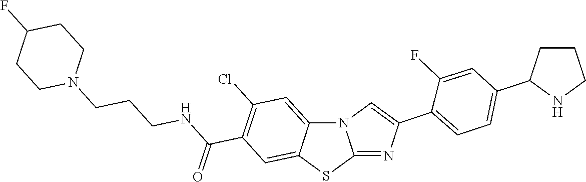 [SMILES: O=C(NCCCN1CCC(F)CC1)c1cc2sc3nc(-c4ccc(C5CCCN5)cc4F)cn3c2cc1Cl]

Compound 6-chloro-2-(2-fluoro-4-(pyrrolidin-2-yl)phenyl)-N-(3-(4-fluoropiperidin-1-yl)propyl)benzo[d]imidazo[2,1-b]thiazole-7-carboxamide dihydrochloride was prepared from tert-butyl 2-(4-(6-chloro-7-((3-(4-fluoropiperidin-1-yl)propyl)carbamoyl)benzo[d]imidazo[2,1-b]thiazol-2-yl)-3-fluorophenyl)pyrrolidine-1-carboxylate (300 mg, 0.456 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride and was isolated as an off-white solid.

Yield 66 mg (23%). ¹H NMR (400 MHz, DMSO) δ 11.18 (br s, 1H), 10.49 (br s, 1H), 9.33 (br s, 1H), 8.82 (d, J=3.6 Hz, 1H), 8.78 (t, J=5.6 Hz, 1H), 8.54 (s, 1H), 8.26 (s, 1H), 8.20-8.15 (m, 1H), 7.65 (dd, J 1.6, 12.4 Hz, 1H), 7.49 (dd, J=1.6, 8.0 Hz, 1H), 5.16-4.72 (m, 1H), 4.65-4.51 (m, 1H), 3.53-3.44 (m, 1H), 3.44-3.23 (m, 6H), 3.20-2.98 (m, 3H), 2.45-2.35 (m, 1H), 2.33-1.92 (m, 9H). ¹⁹F NMR (376 MHz, DMSO) δ −112.82, −175.67, −186.59. m/z: [ESI⁺] 558, 560 (M+H)⁺. (C₂₈H₃₂Cl₃F₂N₅OS).

### Synthesis of (R)-2-(2-fluoro-4-(pyrrolidin-2-yl)phenyl)-6-methoxy-N-(1-methylpiperidin-4-yl)benzo[d]imidazo[2,1-b]thiazole-7-carboxamide Dihydrochloride (Compound 189)

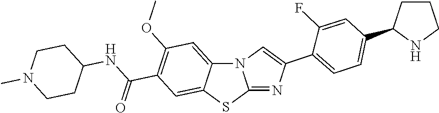 [SMILES: COc1cc2c(cc1C(=O)NC1CCN(C)CC1)sc1nc(-c3ccc([C@H]4CCCN4)cc3F)cn12]

Compound (R)-2-(2-fluoro-4-(pyrrolidin-2-yl)phenyl)-6-methoxy-N-(1-methylpiperidin-4-yl)benzo[d]imidazo[2,1-b]thiazole-7-carboxamide dihydrochloride was prepared from tert-butyl (R)-2-(3-fluoro-4-(6-methoxy-7-((1-methylpiperidin-4-yl)carbamoyl)benzo[d]imidazo[2,1-b]thiazol-2-yl)phenyl)pyrrolidine-1-carboxylate (100 mg, 0.165 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride and was isolated as an off-white solid.

Yield 45 mg (47%). ¹H NMR (400 MHz, DMSO) δ 10.69 (br s, 1H), 10.28 (br s, 1H), 9.16 (br s, 1H), 8.84 (d, J=3.6 Hz, 1H), 8.36-8.28 (m, 1H), 8.24-8.18 (m, 2H), 8.14-8.09 (m, 1H), 7.63 (dd, J=2.0, 12.4 Hz, 1H), 7.48 (dd, J=2.0, 8.4 Hz, 1H), 4.67-4.55 (m, 1H), 4.21-3.96 (m, 4H), 3.47-3.22 (m, 4H), 3.22-2.99 (m, 2H), 2.78-2.68 (m, 3H), 2.46-2.37 (m, 1H), 2.20-1.80 (m, 7H). ¹⁹F NMR (376 MHz, DMSO) δ −113.05. m/z: [ESI⁺] 508 (M+H)⁺. (C27H₃₂Cl₂FN₅O₂S). [α]²⁵_{D}=+8° (c=1 mg/mL, methanol).

### Synthesis of (S)-2-(2-fluoro-4-(pyrrolidin-2-yl)phenyl)-6-methoxy-N-(1-methylpiperidin-4-yl)benzo[d]imidazo[2,1-b]thiazole-7-carboxamide Dihydrochloride (Compound 190)

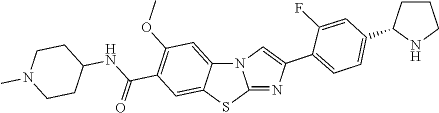 [SMILES: COc1cc2c(cc1C(=O)NC1CCN(C)CC1)sc1nc(-c3ccc([C@@H]4CCCN4)cc3F)cn12]

Compound (S)-2-(2-fluoro-4-(pyrrolidin-2-yl)phenyl)-6-methoxy-N-(1-methylpiperidin-4-yl)benzo[d]imidazo[2,1-b]thiazole-7-carboxamide dihydrochloride was prepared from tert-butyl (S)-2-(3-fluoro-4-(6-methoxy-7-((1-methylpiperidin-4-yl)carbamoyl)benzo[d]imidazo[2,1-b]thiazol-2-yl)phenyl)pyrrolidine-1-carboxylate (110 mg, 0.181 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride and was isolated as an off-white solid.

Yield 47 mg (45%). 1H NMR (400 MHz, DMSO) δ 10.30 (br s, 1H), 10.05 (br s, 1H), 9.02 (br s, 1H), 8.83 (d, J=3.6 Hz, 1H), 8.35-8.27 (m, 1H), 8.25-8.19 (m, 2H), 8.14-8.09 (m, 1H), 7.66-7.55 (m, 1H), 7.51-7.43 (m, 1H), 4.69-4.54 (m, 1H), 4.22-3.95 (m, 4H), 3.51-3.23 (m, 4H), 3.21-2.99 (m, 2H), 2.81-2.70 (m, 3H), 2.45-2.36 (m, 1H), 2.18-1.93 (m, 5H), 1.93-1.75 (m, 2H). 19F NMR (376 MHz, DMSO) δ −113.05. m/z: [ESI⁺] 508 (M+H)⁺. (C27H₃₂Cl₂FN₅O₂S). [α]²⁵_{D}=−2° (c=1 mg/mL, methanol).

### Synthesis of 2-(2-fluoro-4-(4-hydroxypiperidin-4-yl)phenyl)-N-(3-(4-fluoropiperidin-1-yl)propyl)-6-methoxybenzo[d]imidazo[2,1-b]thiazole-7-carboxamide Dihydrochloride (Compound 191)

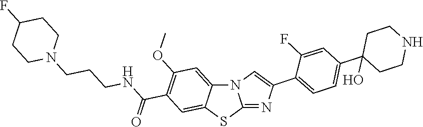 [SMILES: COc1cc2c(cc1C(=O)NCCCN1CCC(F)CC1)sc1nc(-c3ccc(C4(O)CCNCC4)cc3F)cn12]

Compound 2-(2-fluoro-4-(4-hydroxypiperidin-4-yl)phenyl)-N-(3-(4-fluoropiperidin-1-yl)propyl)-6-methoxybenzo[d]imidazo[2,1-b]thiazole-7-carboxamide dihydrochloride was prepared from tert-butyl 4-(3-fluoro-4-(7-((3-(4-fluoropiperidin-1-yl)propyl)carbamoyl)-6-methoxybenzo[d]imidazo[2,1-b]thiazol-2-yl)phenyl)-4-hydroxypiperidine-1-carboxylate (140 mg, 0.205 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride and was isolated as a grey solid.

Yield 115 mg (86%). ¹H NMR (400 MHz, DMSO) δ 10.80 (br s, 1H), 9.12 (br s, 1H), 8.95 (br s, 1H), 8.79 (d, J=3.6 Hz, 1H), 8.50 (t, J=5.6 Hz, 1H), 8.37 (s, 1H), 8.18-8.13 (m, 1H), 8.12 (s, 1H), 7.42-7.36 (m, 2H), 5.11-4.71 (m, 1H), 4.04 (s, 3H), 3.55-3.45 (m, 1H), 3.45-3.33 (m, 3H), 3.26-2.95 (m, 8H), 2.36-1.93 (m, 8H), 1.86-1.73 (m, 2H). OH proton not observed. ¹⁹F NMR (376 MHz, DMSO) δ −113.49, −175.54, −186.61. m/z: [ESI⁺] 584 (M+H)⁺. (C₃₀H37Cl₂F₂N₅O3S).

### Synthesis of 2-(2-fluoro-4-(piperidin-3-yl)phenyl)-N-(3-(4-fluoropiperidin-1-yl)propyl)-6-methoxybenzo[d]imidazo[2,1-b]thiazole-7-carboxamide Dihydrochloride (Compound 192)

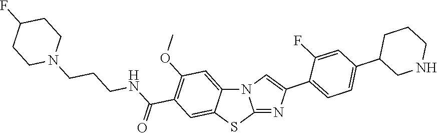 [SMILES: COc1cc2c(cc1C(=O)NCCCN1CCC(F)CC1)sc1nc(-c3ccc(C4CCCNC4)cc3F)cn12]

Compound 2-(2-fluoro-4-(piperidin-3-yl)phenyl)-N-(3-(4-fluoropiperidin-1-yl)propyl)-6-methoxybenzo[d]imidazo[2,1-b]thiazole-7-carboxamide dihydrochloride was prepared from tert-butyl 3-(3-fluoro-4-(7-((3-(4-fluoropiperidin-1-yl)propyl)carbamoyl)-6-methoxybenzo[d]imidazo[2,1-b]thiazol-2-yl)phenyl)piperidine-1-carboxylate (80 mg, 0.120 mmol) following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride and was isolated as a white solid.

Yield 35 mg (46%). ¹H NMR (400 MHz, DMSO) δ 10.81 (br s, 1H), 9.26 (br s, 1H), 9.03 (br s, 1H), 8.78 (d, J=3.6 Hz, 1H), 8.49 (t, J=5.6 Hz, 1H), 8.36 (s, 1H), 8.17-8.06 (m, 2H), 7.39-7.30 (m, 1H), 7.30-7.19 (m, 1H), 5.11-4.70 (m, 1H), 3.88 (s, 3H), 3.56-3.46 (m, 1H), 3.45-3.25 (m, 5H), 3.19-2.94 (m, 6H), 2.94-2.79 (m, 1H), 2.36-1.65 (m, 10H). ¹⁹F NMR (376 MHz, DMSO) δ −113.58, −175.55, −186.61. m/z: [ESI⁺]568 (M+H)⁺. (C₃₀H₃₇Cl₂F₂N₅O₂S).

### Intermediate Preparations

### Synthesis of tert-butyl 3-(3-fluoro-4-(7-((3-(4-fluoropiperidin-1-yl)propyl)carbamoyl)-6-methoxybenzo[d]imidazo[2,1-b]thiazol-2-yl)phenyl)morpholine-4-carboxylate

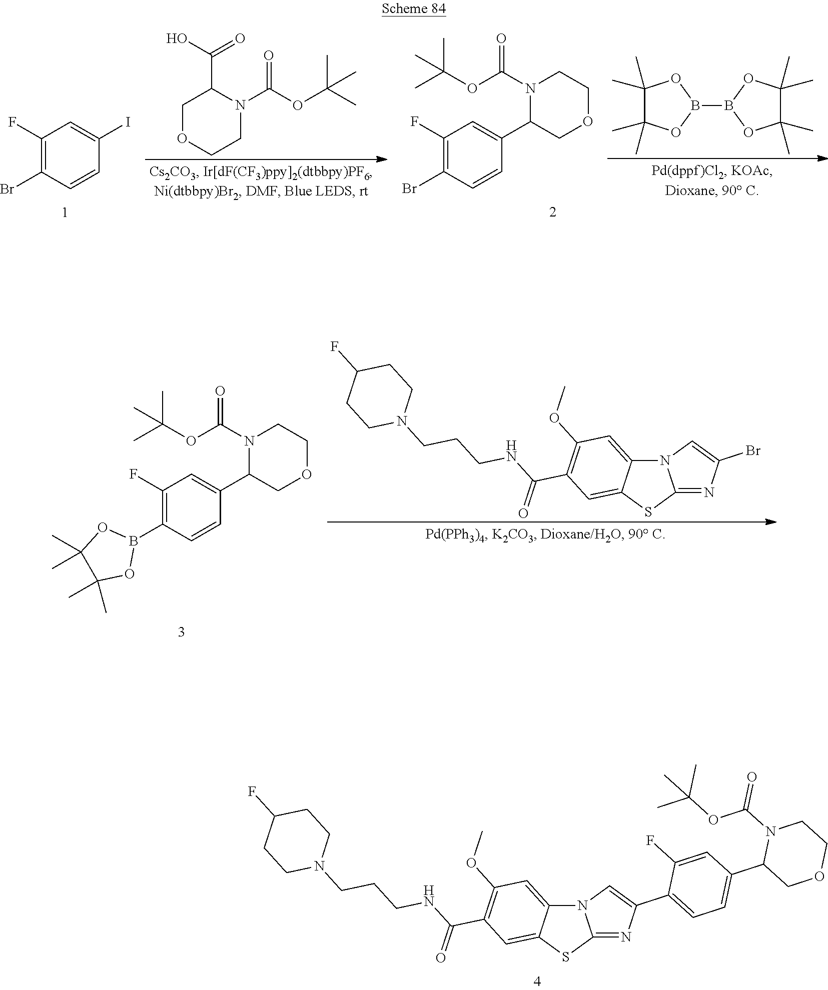

### Synthesis of tert-butyl 3-(4-bromo-3-fluorophenyl)morpholine-4-carboxylate (2, Scheme 84)

A mixture of 1-bromo-2-fluoro-4-iodobenzene (1.00 g, 3.32 mmol), 4-(tert-butoxycarbonyl)morpholine-3-carboxylic acid (1.00 g, 4.32 mmol), cesium carbonate (2.10 g, 6.45 mmol), (4,4′-di-tert-butyl-2,2′-bipyridine)bis[3,5-difluoro-2-[5-trifluoromethyl-2-pyridinyl-κN)phenyl-KC]iridium(I) hexafluorophosphate (Ir[dF(CF₃)ppy]₂(dtbbpy)PF₆) (20 mg, 0.018 mmol) and (SP-4-2)-[4,4′-bis(1,1-dimethylethyl)-2,2′-bipyridine-κN1,κN1′]dibromo-nickel (Ni(dtbbpy)Br₂) (250 mg, 0.513 mmol) in N,N-dimethylformamide (20 mL) was stirred for 4 h at room temperature under a nitrogen atmosphere with irradiation by Blue LED light. The resulting mixture was diluted with water (200 mL) and extracted with ethyl acetate (3×200 mL). The combined organic layers were washed with brine (100 mL) and dried over anhydrous sodium sulfate. After filtration, the filtrate was concentrated under reduced pressure. The residue was purified by silica gel column chromatography (eluted with 0-10% ethyl acetate in petroleum ether) to afford tert-butyl 3-(4-bromo-3-fluorophenyl)morpholine-4-carboxylate as a colorless oil.

Yield 320 mg (27%). ¹H NMR (400 MHz, CDCl₃) δ 7.51 (t, J=7.6 Hz, 1H), 7.31-7.27 (m, 1H), 7.19-7.10 (m, 1H), 5.10-4.94 (m, 1H), 4.33-4.22 (m, 1H), 3.93-3.85 (m, 2H), 3.85-3.75 (m, 1H), 3.62-3.53 (m, 1H), 3.12-3.02 (m, 1H), 1.48 (s, 9H). m/z: [ESI⁺] 304, 306 (M+H−56)⁺.

### Synthesis of tert-butyl 3-(3-fluoro-4-(4,4,5,5-tetramethyl-1,3,2-dioxaborolan-2-yl)phenyl)morpholine-4-carboxylate (3, Scheme 84)

Compound tert-butyl 3-(3-fluoro-4-(4,4,5,5-tetramethyl-1,3,2-dioxaborolan-2-yl)phenyl)morpholine-4-carboxylate was prepared from tert-butyl 3-(4-bromo-3-fluorophenyl)morpholine-4-carboxylate (470 mg, 1.305 mmol) and bis(pinacolato)diboron (500 mg, 1.969 mmol), following a similar procedure to that described for the synthesis of tert-butyl 4-(3-fluoro-4-(4,4,5,5-tetramethyl-1,3,2-dioxaborolan-2-yl)phenyl)piperidine-1-carboxylate and was isolated as a brown solid. The compound was used directly in the next step without further purification.

Yield 450 mg (crude). ¹H NMR not run. m/z: [ESI⁺] 352 (M+H−56)⁺.

### Synthesis of tert-butyl 3-(3-fluoro-4-(7-((3-(4-fluoropiperidin-1-yl)propyl)carbamoyl)-6-methoxybenzo[d]imidazo[2,1-b]thiazol-2-yl)phenyl)morpholine-4-carboxylate (4, Scheme 84)

Compound tert-butyl 3-(3-fluoro-4-(7-((3-(4-fluoropiperidin-1-yl)propyl)carbamoyl)-6-methoxybenzo[d]imidazo[2,1-b]thiazol-2-yl)phenyl)morpholine-4-carboxylate was prepared from 2-bromo-N-(3-(4-fluoropiperidin-1-yl)propyl)-6-methoxybenzo[d]imidazo[2,1-b]thiazole-7-carboxamide (200 mg, 0.426 mmol) and tert-butyl 3-(3-fluoro-4-(4,4,5,5-tetramethyl-1,3,2-dioxaborolan-2-yl)phenyl)morpholine-4-carboxylate (450 mg, crude) at 90° C., following a similar procedure to that described for the synthesis of tert-butyl 4-(4-bromo-3-fluorophenyl)-3,6-dihydropyridine-1(2H)-carboxylate and was isolated as a yellow solid.

Yield 60 mg (21%). ¹H NMR (400 MHz, DMSO) δ 8.59-8.54 (m, 1H), 8.25-8.19 (m, 1H), 8.18-8.09 (m, 1H), 8.08-7.97 (m, 1H), 7.39-7.30 (m, 1H), 7.24-7.21 (m, 1H), 7.12-7.02 (m, 1H), 5.19-5.06 (m, 1H), 4.89-4.73 (m, 1H), 4.75-4.59 (m, 1H), 4.40-4.34 (m, 1H), 4.18-4.10 (m, 4H), 4.00-3.80 (m, 2H), 3.63-3.54 (m, 3H), 2.83-2.52 (m, 4H), 2.06 (d, J=3.2 Hz, 2H), 2.00-1.90 (m, 6H), 1.52 (s, 9H). m/z: [ESI⁺] 670 (M+H)⁺.

### Synthesis of tert-butyl (cis)-2-(3-fluoro-4-(7-((3-(4-fluoropiperidin-1-yl)propyl)carbamoyl)-6-methoxybenzo[d]imidazo[2,1-b]thiazol-2-yl)phenyl)-4-hydroxypyrrolidine-1-carboxylate

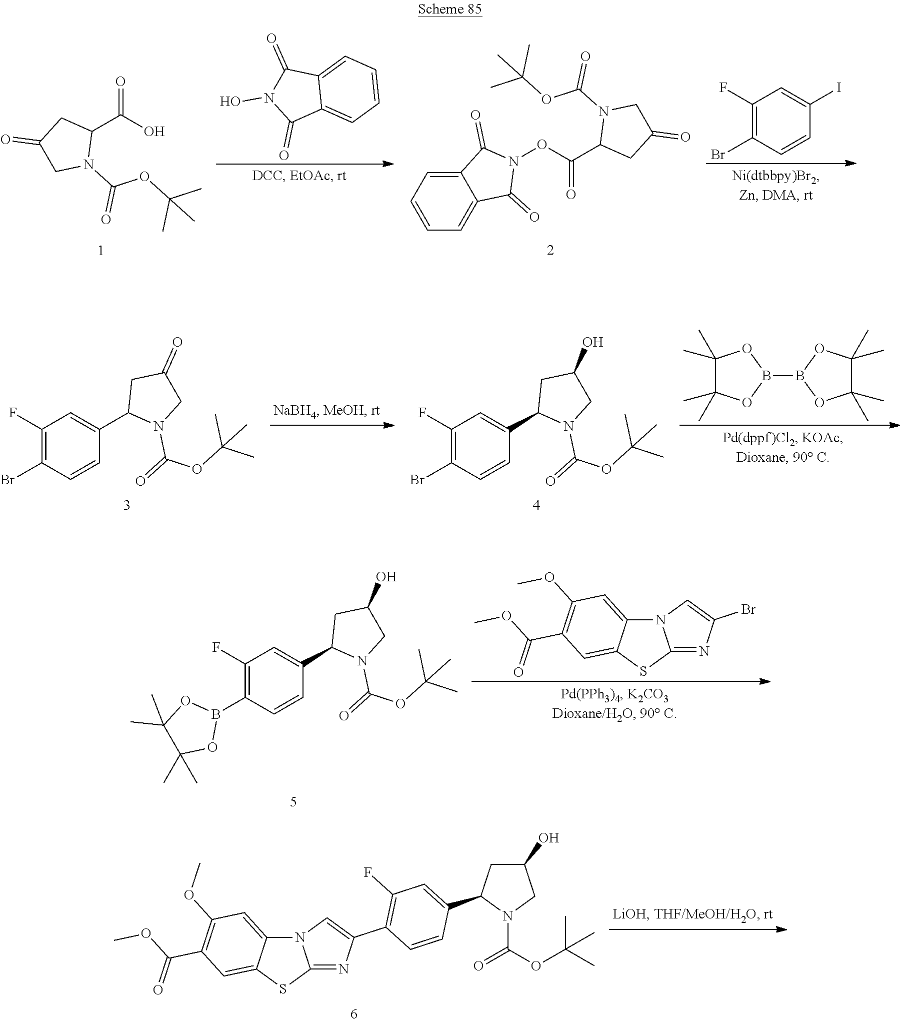

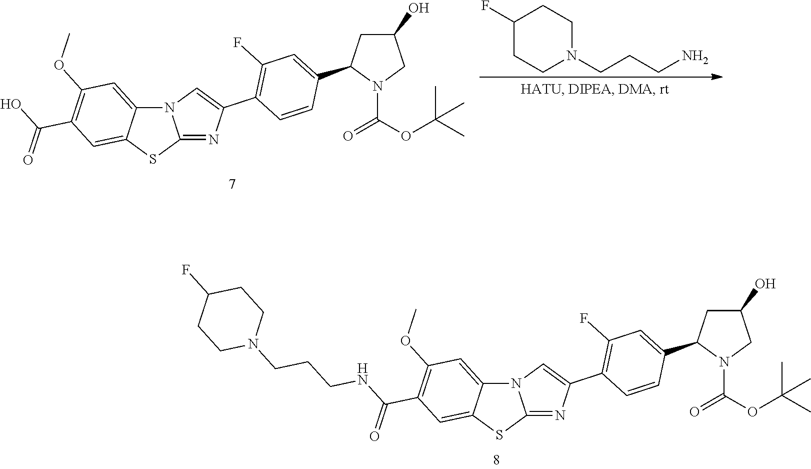 [SMILES: COc1cc2c(cc1C(=O)O)sc1nc(-c3ccc([C@H]4C[C@@H](O)CN4C(=O)OC(C)(C)C)cc3F)cn12 ; NCCCN1CCC(F)CC1 ; COc1cc2c(cc1C(=O)NCCCN1CCC(F)CC1)sc1nc(-c3ccc([C@H]4C[C@@H](O)CN4C(=O)OC(C)(C)C)cc3F)cn12]

### Synthesis of 1-(tert-butyl) 2-(1, 3-dioxoisoindolin-2-yl) 4-oxopyrrolidine-1,2-dicarboxylate (2, Scheme 85)

To a stirred solution of 2-hydroxyisoindoline-1,3-dione (35.60 g, 218.23 mmol) and dicyclohexylmethanediimine (45.00 g, 218.09 mmol) in ethyl acetate (1000 mL) was added a solution of 1-(tert-butoxycarbonyl)-4-oxopyrrolidine-2-carboxylic acid (50.00 g, 218.12 mmol) in ethyl acetate (1000 mL) dropwise over 30 min at room temperature under a nitrogen atmosphere. The reaction solution was stirred for 16 h at room temperature. The resulting mixture was filtered. The filter cake was washed with ethyl acetate (3×300 mL). The combined filtrates were washed with saturated aqueous sodium bicarbonate solution (5×200 mL) and dried over anhydrous sodium sulfate. After filtration, the filtrate was concentrated under reduced pressure to afford 1-(tert-butyl) 2-(1,3-dioxoisoindolin-2-yl) 4-oxopyrrolidine-1,2-dicarboxylate as a white solid.

Yield 30.00 g (37%). ¹H NMR (400 MHz, CDCl₃) δ 7.94-7.88 (m, 2H), 7.88-7.78 (m, 2H), 5.33-5.00 (m, 1H), 4.03-3.91 (m, 2H), 3.30-3.07 (m, 1H), 3.07-2.93 (m, 1H), 1.56 (s, 9H). m/z: [ESI⁺] 397 (M+Na)⁺.

### Synthesis of tert-butyl 2-(4-bromo-3-fluorophenyl)-4-oxopyrrolidine-1-carboxylate (3, Scheme 85)

A mixture of 1-(tert-butyl) 2-(1,3-dioxoisoindolin-2-yl) 4-oxopyrrolidine-1,2-dicarboxylate (30.00 g, 80.14 mmol), 1-bromo-2-fluoro-4-iodobenzene (22.00 g, 73.11 mmol), (SP-4-2)-[4,4′-bis(1,1-dimethylethyl)-2,2′-bipyridine-κN1,κN1′]dibromo-nickel (Ni(dtbbpy)Br₂) (7.80 g, 16.02 mmol) and zinc powder (10.48 g, 160.29 mmol) in N,N-dimethylaniline (240 mL) was stirred for 4 h at room temperature under a nitrogen atmosphere. The reaction mixture was diluted with brine (800 mL) and extracted with ethyl acetate (4×600 mL). The combined organic layers were dried over anhydrous sodium sulfate. After filtration, the filtrate was concentrated under reduced pressure. The residue was purified by silica gel column chromatography (eluted with 1%-20% ethyl acetate in petroleum ether) to afford tert-butyl 2-(4-bromo-3-fluorophenyl)-4-oxopyrrolidine-1-carboxylate as a yellow oil.

Yield 8.50 g (32%). ¹H NMR (400 MHz, CDCl₃) δ 7.54 (dd, J=7.2, 8.0 Hz, 1H), 6.98 (dd, J=2.0, 7.2 Hz, 1H), 6.89 (dd, J=2.0, 8.0 Hz, 1H), 5.42-5.22 (m, 1H), 4.14-4.06 (m, 1H), 3.94-3.84 (m, 1H), 3.30-3.07 (m, 1H), 2.59-2.49 (m, 1H), 1.42 (s, 9H). m/z: [ESI⁺] 302, 304 (M+H−56)⁺.

### Synthesis of tert-butyl (cis)-2-(4-bromo-3-fluorophenyl)-4-hydroxypyrrolidine-1-carboxylate (4, Scheme 85)

Sodium borohydride (1.06 g, 28.02 mmol) was added to a stirred solution of tert-butyl 2-(4-bromo-3-fluorophenyl)-4-oxopyrrolidine-1-carboxylate (5.00 g, 13.96 mmol) in methanol (50 mL) portionwise over 1 min at 0° C. The reaction mixture was stirred for 2 h at room temperature. The reaction was quenched by the addition of acetone (20 mL). The resulting mixture was concentrated under reduced pressure. The residue was purified by silica gel column chromatography (eluted with 12% ethyl acetate in petroleum ether) to afford tert-butyl (cis)-2-(4-bromo-3-fluorophenyl)-4-hydroxypyrrolidine-1-carboxylate as a colorless oil.

Yield 2.50 g (50%). ¹H NMR (400 MHz, CDCl₃) δ 7.49 (dd, J=7.2, 8.4 Hz, 1H), 7.11 (dd, J=2.0, 9.6 Hz, 1H), 6.99 (dd, J=2.0, 8.4 Hz, 1H), 5.01-4.75 (m, 1H), 4.56-4.45 (m, 1H), 3.94-3.76 (m, 1H), 3.65-3.53 (m, 1H), 2.65-2.54 (m, 1H), 2.01-1.92 (m, 1H), 1.48-1.21 (m, 9H). OH proton not observed. m/z: [ESI⁺] 304, 306 (M+H−56)⁺.

### Synthesis of tert-butyl (cis)-2-(3-fluoro-4-(4,4,5,5-tetramethyl-1,3,2-dioxaborolan-2-yl)phenyl)-4-hydroxypyrrolidine-1-carboxylate (5, Scheme 85)

Compound tert-butyl (cis)-2-(3-fluoro-4-(4,4,5,5-tetramethyl-1,3,2-dioxaborolan-2-yl)phenyl)-4-hydroxypyrrolidine-1-carboxylate was prepared from tert-butyl (cis)-2-(4-bromo-3-fluorophenyl)-4-hydroxypyrrolidine-1-carboxylate (900 mg, 2.498 mmol) and bis(pinacolato)diboron (950 mg, 3.741 mmol), following a similar procedure to that described for the synthesis of tert-butyl 3-(3-fluoro-4-(4,4,5,5-tetramethyl-1,3,2-dioxaborolan-2-yl)phenyl)pyrrolidine-1-carboxylate and was isolated as a yellow solid.

Yield 750 mg (crude). ¹H NMR not run. m/z: [ESI⁺] 352 (M+H−56)⁺.

### Synthesis of methyl 2-(4-((cis)-1-(tert-butoxycarbonyl)-4-hydroxypyrrolidin-2-yl)-2-fluorophenyl)-6-methoxybenzo[d]imidazo[2,1-b]thiazole-7-carboxylate (6, Scheme 85)

Compound methyl 2-(4-((cis)-1-(tert-butoxycarbonyl)-4-hydroxypyrrolidin-2-yl)-2-fluorophenyl)-6-methoxybenzo[d]imidazo[2,1-b]thiazole-7-carboxylate was prepared from methyl 2-bromo-6-methoxybenzo[d]imidazo[2,1-b]thiazole-7-carboxylate (1.68 g, 4.92 mmol) and tert-butyl (cis)-2-(3-fluoro-4-(4,4,5,5-tetramethyl-1,3,2-dioxaborolan-2-yl)phenyl)-4-hydroxypyrrolidine-1-carboxylate (2.00 g, crude) at 90° C., following a similar procedure to that described for the synthesis of tert-butyl 4-(4-bromo-3-fluorophenyl)-3,6-dihydropyridine-1(2H)-carboxylate and was isolated as a yellow solid.

Yield 1.50 g (56%). ¹H NMR (300 MHz, CDCl₃) δ 8.23 (s, 1H), 8.19-8.14 (m, 2H), 7.24-7.16 (m, 1H), 7.15-7.10 (m, 2H), 4.66-4.44 (m, 1H), 4.12-4.00 (m, 3H), 4.01-3.93 (m, 4H), 3.67-3.56 (m, 1H), 2.80-2.49 (m, 2H), 2.16-1.98 (m, 1H), 1.49-1.30 (m, 9H). OH proton not observed. m/z: [ESI⁺] 542 (M+H)⁺.

### Synthesis of 2-(4-((cis)-1-(tert-butoxycarbonyl)-4-hydroxypyrrolidin-2-yl)-2-fluorophenyl)-6-methoxybenzo[d]imidazo[2,1-b]thiazole-7-carboxylic Acid (7, Scheme 85)

Compound 2-(4-((cis)-1-(tert-butoxycarbonyl)-4-hydroxypyrrolidin-2-yl)-2-fluorophenyl)-6-methoxybenzo[d]imidazo[2,1-b]thiazole-7-carboxylic acid was prepared from methyl 2-(4-((cis)-1-(tert-butoxycarbonyl)-4-hydroxypyrrolidin-2-yl)-2-fluorophenyl)-6-methoxybenzo[d]imidazo[2,1-b]thiazole-7-carboxylate (240 mg, 0.443 mmol) at room temperature, following a similar procedure to that described for the synthesis of 2-(1-(tert-butoxycarbonyl)-6-fluoroindolin-5-yl)benzo[d]imidazo[2,1-b]thiazole-7-carboxylic acid and was isolated as a white solid.

Yield 200 mg (86%). ¹H NMR (400 MHz, DMSO) δ 12.90 (br s, 1H), 8.87-8.45 (m, 1H), 8.34-8.15 (m, 1H), 8.17-7.94 (m, 2H), 7.30-7.04 (m, 2H), 5.18-5.00 (m, 1H), 4.89-4.63 (m, 1H), 4.35-4.17 (m, 1H), 3.97-3.90 (m, 3H), 3.72-3.57 (m, 1H), 3.32-3.28 (m, 1H), 1.80-1.70 (m, 1H), 1.40-1.11 (m, 9H). OH proton not observed. m/z: [ESI⁺] 528 (M+H)⁺.

### Synthesis of tert-butyl (cis)-2-(3-fluoro-4-(7-((3-(4-fluoropiperidin-1-yl)propyl)carbamoyl)-6-methoxybenzo[d]imidazo[2,1-b]thiazol-2-yl)phenyl)-4-hydroxypyrrolidine-1-carboxylate (8, Scheme 85)

Compound tert-butyl (cis)-2-(3-fluoro-4-(7-((3-(4-fluoropiperidin-1-yl)propyl)carbamoyl)-6-methoxybenzo[d]imidazo[2,1-b]thiazol-2-yl)phenyl)-4-hydroxypyrrolidine-1-carboxylate was prepared from 2-(4-((cis)-1-(tert-butoxycarbonyl)-4-hydroxypyrrolidin-2-yl)-2-fluorophenyl)-6-methoxybenzo[d]imidazo[2,1-b]thiazole-7-carboxylic acid (50 mg, 0.095 mmol) and 3-(4-fluoropiperidin-1-yl)propan-1-amine (30 mg, 0.187 mmol), following a similar procedure to that described for the synthesis of tert-butyl 6-fluoro-5-(7-((3-(4-fluoropiperidin-1-yl)propyl)carbamoyl)benzo[d]imidazo[2,1-b]thiazol-2-yl)indoline-1-carboxylate and was isolated as a yellow solid.

Yield 30 mg (47%). ¹H NMR (400 MHz, DMSO) δ 8.75-8.72 (m, 1H), 8.34-8.31 (m, 1H), 8.26 (t, J=5.6 Hz, 1H), 8.12-8.03 (m, 2H), 7.26-7.16 (m, 2H), 5.18-4.95 (m, 1H), 4.78-4.50 (m, 2H), 4.32-4.23 (m, 1H), 4.14-4.05 (m, 1H), 4.02 (s, 3H), 3.74-3.60 (m, 1H), 3.21-3.13 (m, 4H), 2.82-2.69 (m, 1H), 2.41-2.17 (m, 6H), 1.75-1.63 (m, 4H), 1.40-1.11 (m, 9H). OH proton not observed. m/z: [ESI⁺] 670 (M+H)⁺.

### Synthesis of tert-butyl (cis)-2-(3-fluoro-4-(6-methoxy-7-((1-methylpiperidin-4-yl)carbamoyl)benzo[d]imidazo[2,1-b]thiazol-2-yl)phenyl)-4-hydroxypyrrolidine-1-carboxylate

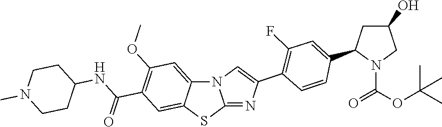 [SMILES: COc1cc2c(cc1C(=O)NC1CCN(C)CC1)sc1nc(-c3ccc([C@H]4C[C@@H](O)CN4C(=O)OC(C)(C)C)cc3F)cn12]

Compound tert-butyl (cis)-2-(3-fluoro-4-(6-methoxy-7-((1-methylpiperidin-4-yl)carbamoyl)benzo[d]imidazo[2,1-b]thiazol-2-yl)phenyl)-4-hydroxypyrrolidine-1-carboxylate was prepared from 2-(4-((cis)-1-(tert-butoxycarbonyl)-4-hydroxypyrrolidin-2-yl)-2-fluorophenyl)-6-methoxybenzo[d]imidazo[2,1-b]thiazole-7-carboxylic acid (50 mg, 0.095 mmol) and 1-methylpiperidin-4-amine (22 mg, 0.193 mmol), following a similar procedure to that described for the synthesis of tert-butyl 6-fluoro-5-(7-((3-(4-fluoropiperidin-1-yl)propyl)carbamoyl)benzo[d]imidazo[2,1-b]thiazol-2-yl)indoline-1-carboxylate and was isolated as a white solid.

Yield 30 mg (51%). ¹H NMR not run. m/z: [ESI⁺] 624 (M+H)⁺.

### Synthesis of tert-butyl (trans)-2-(3-fluoro-4-(7-((3-(4-fluoropiperidin-1-yl)propyl)carbamoyl)-6-methoxybenzo[d]imidazo[2,1-b]thiazol-2-yl)phenyl)-4-hydroxypyrrolidine-1-carboxylate

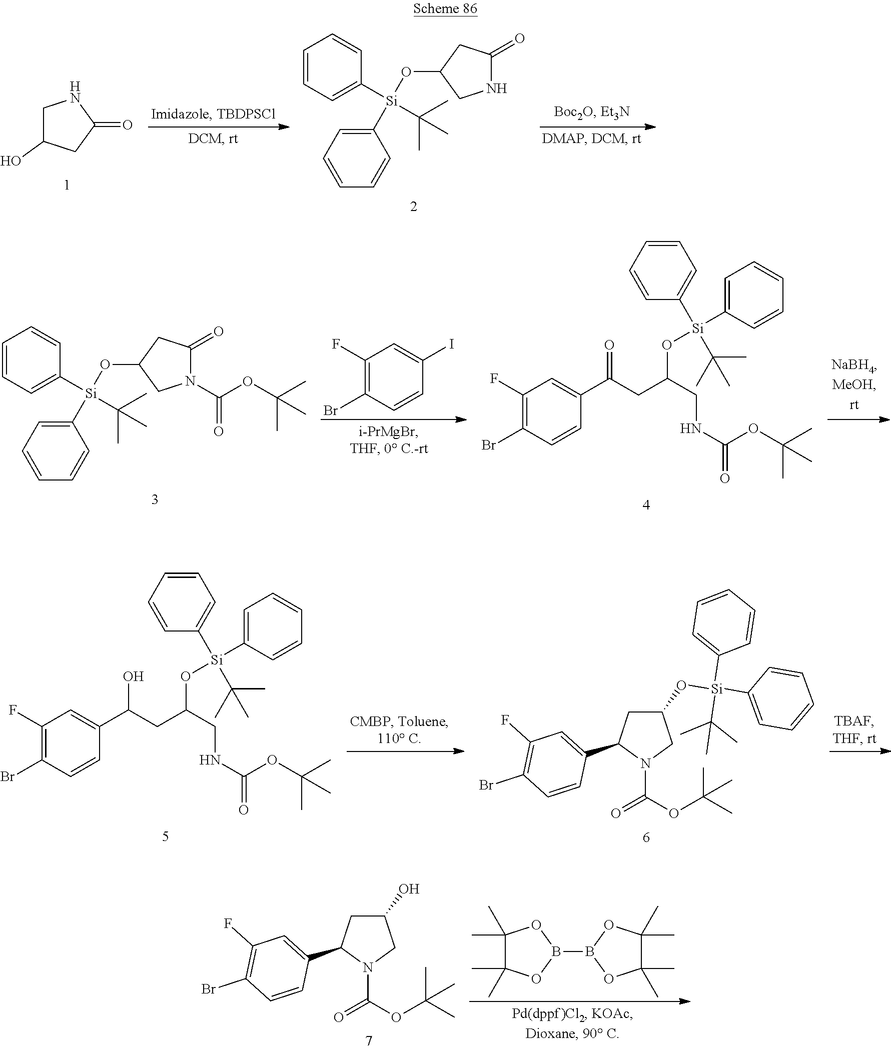

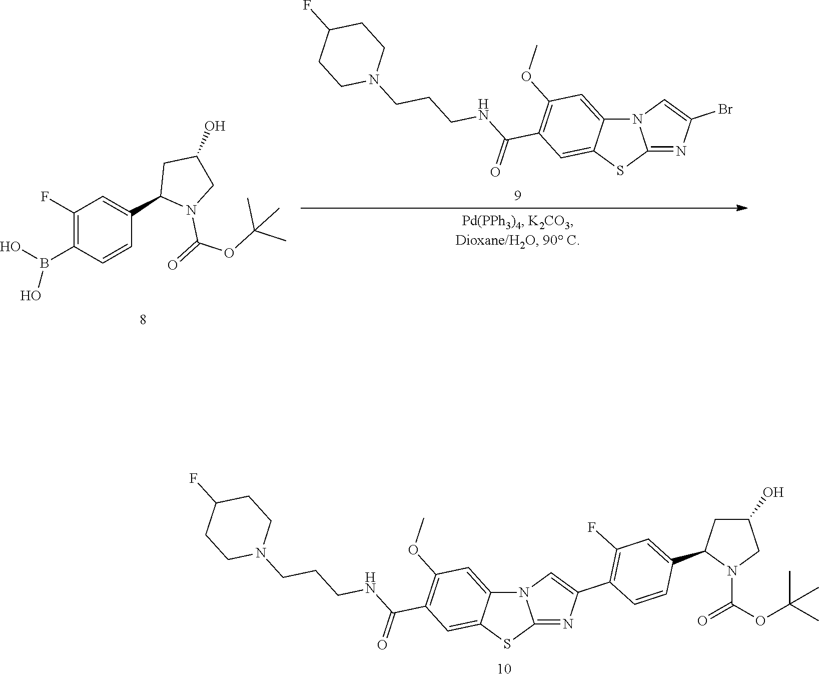 [SMILES: CC(C)(C)OC(=O)N1C[C@@H](O)C[C@@H]1c1ccc(B(O)O)c(F)c1 ; COc1cc2c(cc1C(=O)NCCCN1CCC(F)CC1)sc1nc(Br)cn12 ; COc1cc2c(cc1C(=O)NCCCN1CCC(F)CC1)sc1nc(-c3ccc([C@H]4C[C@H](O)CN4C(=O)OC(C)(C)C)cc3F)cn12]

### Synthesis of 4-((tert-butyldiphenylsilyl)oxy)pyrrolidin-2-one (2, Scheme 86)

To a stirred solution of 4-hydroxypyrrolidin-2-one (15.00 g, 148.35 mmol) in dichloromethane (200 mL) were added imidazole (15.00 g, 220.33 mmol) and tert-butylchlorodiphenylsilane (53.00 g, 192.83 mmol) sequentially as single portions at 0° C. under a nitrogen atmosphere. The reaction solution was stirred for 2 h at room temperature. The resulting mixture was diluted with water (400 mL) and extracted with dichloromethane (2×200 mL). The combined organic layers were washed with water (200 mL) and dried over anhydrous sodium sulfate. After filtration, the filtrate was concentrated under reduced pressure. The residue was purified by silica gel column chromatography (eluted with 30% ethyl acetate in petroleum ether) to afford 4-((tert-butyldiphenylsilyl)oxy)pyrrolidin-2-one as a white solid.

Yield 38.00 g (75%). ¹H NMR (400 MHz, CDCl₃) δ 7.69-7.60 (m, 4H), 7.50-7.38 (m, 6H), 5.99 (br s, 1H), 4.62-4.50 (m, 1H), 3.44-3.36 (m, 1H), 3.36-3.27 (m, 1H), 2.47-2.34 (m, 2H), 1.09 (s, 9H). m/z: [ESI⁺] 340 (M+H)⁺.

### Synthesis of tert-butyl 4-((tert-butyldiphenylsilyl)oxy)-2-oxopyrrolidine-1-carboxylate (3, Scheme 86)

To a stirred solution of 4-((tert-butyldiphenylsilyl)oxy)pyrrolidin-2-one (20.00 g, 58.91 mmol) and N,N-diisopropylethylamine (15.00 g, 116.05 mmol) in dichloromethane (400 mL) were added 4-dimethylaminopyridine (0.70 g, 5.73 mmol) and di-tert-butyl dicarbonate (20.00 g, 91.64 mmol) sequentially as single portions at room temperature under a nitrogen atmosphere. The reaction solution was stirred for 16 h at room temperature. The resulting mixture was concentrated under reduced pressure. The residue was purified by silica gel column chromatography (eluted with 30% ethyl acetate in petroleum ether) to afford tert-butyl 4-((tert-butyldiphenylsilyl)oxy)-2-oxopyrrolidine-1-carboxylate as a colorless oil.

Yield 25.00 g (97%). ¹H NMR (300 MHz, CDCl₃) δ 7.68-7.61 (m, 4H), 7.51-7.38 (m, 6H), 4.43-4.34 (m, 1H), 3.73-3.66 (m, 2H), 2.58-2.52 (m, 2H), 1.54 (s, 9H), 1.08 (s, 9H). m/z: [ESI⁺] 384 (M+H−56)⁺.

### Synthesis of tert-butyl (4-(4-bromo-3-fluorophenyl)-2-((tert-butyldiphenylsilyl)oxy)-4-oxobutyl)carbamate (4, Scheme 86)

Compound tert-butyl (4-(4-bromo-3-fluorophenyl)-2-((tert-butyldiphenylsilyl)oxy)-4-oxobutyl)carbamate was prepared from 1-bromo-2-fluoro-4-iodobenzene (13.00 g, 43.20 mmol) and tert-butyl 4-((tert-butyldiphenylsilyl)oxy)-2-oxopyrrolidine-1-carboxylate (25.00 g, 56.87 mmol), following a similar procedure to that described for the synthesis of tert-butyl (4-(4-bromo-2,3-difluorophenyl)-4-oxobutyl)carbamate and was isolated as a colorless oil.

Yield 11.00 g (41%). ¹H NMR (400 MHz, DMSO) δ 7.84-7.79 (m, 1H), 7.67-7.62 (m, 1H), 7.62-7.49 (m, 4H), 7.47-7.29 (m, 7H), 6.83-6.77 (m, 1H), 4.43-4.34 (m, 1H), 3.22-3.02 (m, 4H), 1.32 (s, 9H), 0.93 (s, 9H). m/z: [ESI⁺] 636, 638 (M+Na)⁺.

### Synthesis of tert-butyl (4-(4-bromo-3-fluorophenyl)-2-((tert-butyldiphenylsilyl)oxy)-4-hydroxybutyl)carbamate (5, Scheme 86)

Compound tert-butyl (4-(4-bromo-3-fluorophenyl)-2-((tert-butyldiphenylsilyl)oxy)-4-hydroxybutyl)carbamate was prepared from tert-butyl (4-(4-bromo-3-fluorophenyl)-2-((tert-butyldiphenylsilyl)oxy)-4-oxobutyl)carbamate (90.00 g, 146.43 mmol) and sodium tetrahydroborate (8.31 g, 219.67 mmol), following a similar procedure to that described for the synthesis of tert-butyl (cis)-2-(4-bromo-3-fluorophenyl)-4-hydroxypyrrolidine-1-carboxylate and was isolated as a colorless oil.

Yield 80.00 g (89%). ¹H NMR (400 MHz, CDCl₃) δ 7.75-7.70 (m, 1H), 7.70-7.62 (m, 4H), 7.52-7.37 (m, 6H), 7.02-6.95 (m, 1H), 6.86-6.80 (m, 1H), 4.90-4.82 (m, 1H), 4.78-4.69 (m, 1H), 4.06-4.00 (m, 1H), 3.42-3.32 (m, 2H), 3.29-3.19 (m, 1H), 1.92-1.75 (m, 2H), 1.44 (s, 9H), 1.10 (s, 9H). m/z: [ESI⁺]616, 618 (M+1)⁺.

### Synthesis of tert-butyl (trans)-2-(4-bromo-3-fluorophenyl)-4-((tert-butyldiphenylsilyl)oxy)pyrrolidine-1-carboxylate (6, Scheme 86)

To a stirred solution of tert-butyl (4-(4-bromo-3-fluorophenyl)-2-((tert-butyldiphenylsilyl)oxy)-4-hydroxybutyl)carbamate (30.00 g, 48.65 mmol) in toluene (300 mL) was added cyanomethylenetributylphosphorane (25.00 g, 103.58 mmol) dropwise over 5 min at room temperature under a nitrogen atmosphere. The reaction solution was stirred for 16 h at 110° C. The resulting mixture was cooled to room temperature and concentrated under reduced pressure. The residue was purified by silica gel column chromatography (eluted with 5% ethyl acetate in petroleum ether) to afford tert-butyl (trans)-2-(4-bromo-3-fluorophenyl)-4-((tert-butyldiphenylsilyl)oxy)pyrrolidine-1-carboxylate as a colorless oil.

Yield 17.00 g (58%). ¹H NMR (400 MHz, DMSO) δ 7.69-7.63 (m, 1H), 7.56-7.33 (m, 10H), 7.30-7.18 (m, 1H), 7.13-7.05 (m, 1H), 4.91-4.75 (m, 1H), 4.42-4.33 (m, 1H), 3.63-3.50 (m, 1H), 3.50-3.39 (m, 1H), 2.50-2.35 (m, 1H), 1.93-1.83 (m, 1H), 1.40-1.13 (m, 9H), 0.87 (s, 9H). m/z: [ESI⁺] 598, 600 (M+1)⁺.

### Synthesis of tert-butyl (trans)-2-(4-bromo-3-fluorophenyl)-4-hydroxypyrrolidine-1-carboxylate (7, Scheme 86)

Tetrabutylammonium fluoride (8.74 g, 33.43 mmol) was added to a stirred solution of tert-butyl (trans)-2-(4-bromo-3-fluorophenyl)-4-((tert-butyldiphenylsilyl)oxy)pyrrolidine-1-carboxylate (10.00 g, 16.70 mmol) in tetrahydrofuran (100 mL) portionwise over 2 min at room temperature. The reaction solution was stirred for 2 h. The resulting mixture was concentrated under reduced pressure. The residue was purified by silica gel column chromatography (eluted with 15% ethyl acetate in petroleum ether) to afford tert-butyl (trans)-2-(4-bromo-3-fluorophenyl)-4-hydroxypyrrolidine-1-carboxylate as a white solid.

Yield 3.00 g (50%). ¹H NMR (400 MHz, CDCl₃) δ 7.74-7.63 (m, 1H), 7.10-7.02 (m, 1H), 7.00-6.95 (m, 1H), 5.05-4.78 (m, 1H), 4.54-4.41 (m, 1H), 3.95-3.81 (m, 1H), 3.64-3.55 (m, 1H), 2.66-2.54 (m, 1H), 2.03-1.93 (m, 1H), 1.41-1.22 (m, 9H). OH proton not observed. m/z: [ESI⁺] 304, 306 (M+1-56)⁺.

### Synthesis of (4-((trans)-1-(tert-butoxycarbonyl)-4-hydroxypyrrolidin-2-yl)-2-fluorophenyl)boronic Acid (8, Scheme 86)

Compound (4-((trans)-1-(tert-butoxycarbonyl)-4-hydroxypyrrolidin-2-yl)-2-fluorophenyl)boronic acid was prepared from tert-butyl (trans)-2-(4-bromo-3-fluorophenyl)-4-hydroxypyrrolidine-1-carboxylate (200 mg, 0.555 mmol) and bis(pinacolato)diboron (210 mg, 0.827 mmol), following a similar procedure to that described for the synthesis of tert-butyl 4-(3-fluoro-4-(4,4,5,5-tetramethyl-1,3,2-dioxaborolan-2-yl)phenyl)piperidine-1-carboxylate and was isolated as an off-white solid.

Yield 130 mg (72%). ¹H NMR (400 MHz, DMSO) δ 7.62-7.54 (m, 1H), 7.09-7.02 (m, 1H), 7.02-6.95 (m, 1H), 5.09-4.97 (m, 1H), 4.86-4.73 (m, 1H), 4.33-4.19 (m, 1H), 3.67-3.53 (m, 1H), 3.51-3.41 (m, 1H), 2.29-2.18 (m, 1H), 1.82-1.68 (m, 1H), 1.31-1.09 (m, 9H). Two OH protons of boronic acid not observed. m/z: [ESI⁺] 270 (M+1-56)⁺.

### Synthesis of tert-butyl (trans)-2-(3-fluoro-4-(7-((3-(4-fluoropiperidin-1-yl)propyl)carbamoyl)-6-methoxybenzo[d]imidazo[2,1-b]thiazol-2-yl)phenyl)-4-hydroxypyrrolidine-1-carboxylate (10, Scheme 86)

Compound tert-butyl (trans)-2-(3-fluoro-4-(7-((3-(4-fluoropiperidin-1-yl)propyl)carbamoyl)-6-methoxybenzo[d]imidazo[2,1-b]thiazol-2-yl)phenyl)-4-hydroxypyrrolidine-1-carboxylate was prepared from 2-bromo-N-(3-(4-fluoropiperidin-1-yl)propyl)-6-methoxybenzo[d]imidazo[2,1-b]thiazole-7-carboxamide (70 mg, 0.149 mmol) and (4-((trans)-1-(tert-butoxycarbonyl)-4-hydroxypyrrolidin-2-yl)-2-fluorophenyl)boronic acid (80 mg, 0.246 mmol), following a similar procedure to that described for the synthesis of tert-butyl 6-fluoro-5-(7-((3-(4-fluoropiperidin-1-yl)propyl)carbamoyl)benzo[d]imidazo[2,1-b]thiazol-2-yl)indoline-1-carboxylate and was isolated as a brown solid.

Yield 50 mg (50%). ¹H NMR (300 MHz, DMSO) δ 8.80-8.64 (m, 1H), 8.37-8.29 (m, 1H), 8.26 (t, J=5.7 Hz, 1H), 8.19-8.03 (m, 2H), 7.62-7.45 (m, 1H), 7.23-7.11 (m, 1H), 5.15-4.95 (m, 1H), 4.93-4.67 (m, 2H), 4.65-4.46 (m, 1H), 4.33-4.25 (m, 1H), 4.02 (s, 3H), 3.71-3.41 (m, 2H), 2.41-2.22 (m, 6H), 2.00-1.60 (m, 8H), 1.39-1.10 (m, 9H). OH proton not observed. m/z: [ESI⁺] 670 (M+H)⁺.

### Synthesis of tert-butyl (trans)-2-(3-fluoro-4-(6-methoxy-7-((1-methylpiperidin-4-yl)carbamoyl)benzo[d]imidazo[2,1-b]thiazol-2-yl)phenyl)-4-hydroxypyrrolidine-1-carboxylate

Compound tert-butyl (trans)-2-(3-fluoro-4-(6-methoxy-7-((1-methylpiperidin-4-yl)carbamoyl)benzo[d]imidazo[2,1-b]thiazol-2-yl)phenyl)-4-hydroxypyrrolidine-1-carboxylate was prepared from 2-bromo-6-methoxy-N-(1-methylpiperidin-4-yl)benzo[d]imidazo[2,1-b]thiazole-7-carboxamide (50 mg, 0.118 mmol) and (4-((trans)-1-(tert-butoxycarbonyl)-4-hydroxypyrrolidin-2-yl)-2-fluorophenyl)boronic acid (60 mg, 0.185 mmol), following a similar procedure to that described for the synthesis of tert-butyl 6-fluoro-5-(7-((3-(4-fluoropiperidin-1-yl)propyl)carbamoyl)benzo[d]imidazo[2,1-b]thiazol-2-yl)indoline-1-carboxylate and was isolated as a brown solid.

Yield 50 mg (68%). ¹H NMR not run. m/z: [ESI⁺] 624 (M+H)⁺.

### Synthesis of tert-butyl 3-(3-fluoro-4-(7-((3-(4-fluoropiperidin-1-yl)propyl)carbamoyl)-6-methoxybenzo[d]imidazo[2,1-b]thiazol-2-yl)phenyl)-3-hydroxypyrrolidine-1-carboxylate

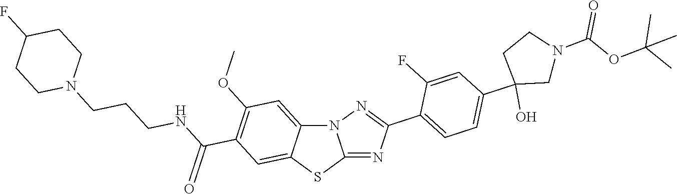 [SMILES: COc1cc2c(cc1C(=O)NCCCN1CCC(F)CC1)sc1nc(-c3ccc(C4(O)CCN(C(=O)OC(C)(C)C)C4)cc3F)nn12]

Compound tert-butyl 3-(3-fluoro-4-(7-((3-(4-fluoropiperidin-1-yl)propyl)carbamoyl)-6-methoxybenzo[d]imidazo[2,1-b]thiazol-2-yl)phenyl)-3-hydroxypyrrolidine-1-carboxylate was prepared from 2-bromo-N-(3-(4-fluoropiperidin-1-yl)propyl)-6-methoxybenzo[d]imidazo[2,1-b]thiazole-7-carboxamide (200 mg, 0.426 mmol) and tert-butyl 3-(3-fluoro-4-(4,4,5,5-tetramethyl-1,3,2-dioxaborolan-2-yl)phenyl)-3-hydroxypyrrolidine-1-carboxylate (350 mg, crude) at 90° C., following a similar procedure to that described for the synthesis of tert-butyl 4-(4-bromo-3-fluorophenyl)-3,6-dihydropyridine-1(2H)-carboxylate and was isolated as a brown yellow solid.

Yield 180 mg (63%). ¹H NMR (300 MHz, CDCl₃) δ 8.38-8.20 (m, 1H), 8.02 (t, J=5.7 Hz, 1H), 7.96-7.84 (m, 2H), 7.26-7.16 (m, 2H), 7.11-7.06 (m, 1H), 4.95-4.61 (m, 1H), 4.11 (s, 3H), 3.80-3.48 (m, 6H), 2.81-2.51 (m, 6H), 2.13-1.84 (m, 8H), 1.51 (s, 9H). OH proton not observed. m/z: [ESI⁺] 670 (M+H)⁺.

### Final Compounds

### Synthesis of 2-(2-fluoro-4-(morpholin-3-yl)phenyl)-N-(3-(4-fluoropiperidin-1-yl)propyl)-6-methoxybenzo[d]imidazo[2,1-b]thiazole-7-carboxamide Dihydrochloride (Compound 195)

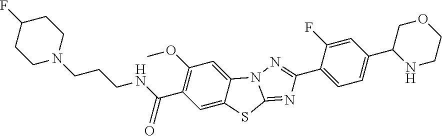 [SMILES: COc1cc2c(cc1C(=O)NCCCN1CCC(F)CC1)sc1nc(-c3ccc(C4COCCN4)cc3F)nn12]

Compound 2-(2-fluoro-4-(morpholin-3-yl)phenyl)-N-(3-(4-fluoropiperidin-1-yl)propyl)-6-methoxybenzo[d]imidazo[2,1-b]thiazole-7-carboxamide dihydrochloride was prepared from tert-butyl 3-(3-fluoro-4-(7-((3-(4-fluoropiperidin-1-yl)propyl)carbamoyl)-6-methoxybenzo[d]imidazo[2,1-b]thiazol-2-yl)phenyl)morpholine-4-carboxylate (60 mg, 0.090 mmol), following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride and was isolated as an white solid.

Yield 19 mg (33%). ¹H NMR (400 MHz, DMSO) δ 10.86 (br s, 1H), 10.31 (br s, 1H), 9.98 (br s, 1H), 8.85 (d, J=3.6 Hz, 1H), 8.50 (t, J=5.6 Hz, 1H), 8.37 (s, 1H), 8.25-8.16 (m, 1H), 8.13 (s, 1H), 7.77 (d, J=12.4 Hz, 1H), 7.56 (d, J=8.0 Hz, 1H), 5.18-4.67 (m, 1H), 4.60-4.49 (m, 1H), 4.02-3.93 (m, 6H), 3.94-3.81 (m, 2H), 3.59-3.20 (m, 5H), 3.14-2.99 (m, 4H), 2.33-1.89 (m, 6H). ¹⁹F NMR (376 MHz, DMSO) δ −112.27, −175.52, −186.58. m/z: [ESI⁺] 570 (M+H)⁺. (C₂₉H₃₅Cl₂F₂N₅O₃S).

### Synthesis of 2-(2-fluoro-4-(3-hydroxypyrrolidin-3-yl)phenyl)-N-(3-(4-fluoropiperidin-1-yl)propyl)-6-methoxybenzo[d]imidazo[2,1-b]thiazole-7-carboxamide Dihydrochloride (Compound 197)

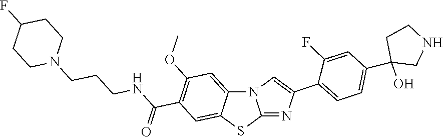 [SMILES: COc1cc2c(cc1C(=O)NCCCN1CCC(F)CC1)sc1nc(-c3ccc(C4(O)CCNC4)cc3F)cn12]

Compound 2-(2-fluoro-4-(3-hydroxypyrrolidin-3-yl)phenyl)-N-(3-(4-fluoropiperidin-1-yl)propyl)-6-methoxybenzo[d]imidazo[2,1-b]thiazole-7-carboxamide dihydrochloride was prepared from tert-butyl 3-(3-fluoro-4-(7-((3-(4-fluoropiperidin-1-yl)propyl)carbamoyl)-6-methoxybenzo[d]imidazo[2,1-b]thiazol-2-yl)phenyl)-3-hydroxypyrrolidine-1-carboxylate (180 mg, 0.269 mmol), following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride and was isolated as a brown yellow solid.

Yield 140 mg (81%). ¹H NMR (400 MHz, DMSO) δ 11.01 (br s, 1H), 9.84 (br s, 1H), 9.46 (br s, 1H), 8.94-8.68 (m, 1H), 8.66-8.46 (m, 1H), 8.37 (d, J=1.6 Hz, 1H), 8.20-8.05 (m, 2H), 7.64-7.31 (m, 2H), 5.17-4.67 (m, 1H), 4.05 (s, 3H), 3.57-3.32 (m, 8H), 3.22-3.00 (m, 4H), 2.41-1.89 (m, 8H). OH proton not observed. ¹⁹F NMR (376 MHz, DMSO) δ −113.45, −175.53, −186.56. m/z: [ESI⁺] 570 (M+H)⁺. (C₂₉H₃₅Cl₂F₂N₅O3S).

### Synthesis of 2-(2-fluoro-4-((2S,4S)-4-hydroxypyrrolidin-2-yl)phenyl)-N-(3-(4-fluoropiperidin-1-yl)propyl)-6-methoxybenzo[d]imidazo[2,1-b]thiazole-7-carboxamide (Compound 200S)

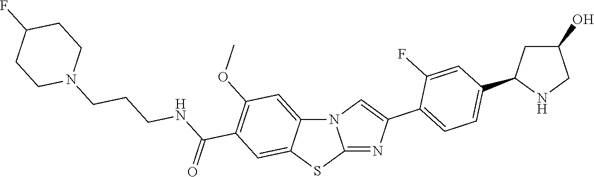 [SMILES: COc1cc2c(cc1C(=O)NCCCN1CCC(F)CC1)sc1nc(-c3ccc([C@H]4C[C@@H](O)CN4)cc3F)cn12]

Compound 2-(2-fluoro-4-((cis)-4-hydroxypyrrolidin-2-yl)phenyl)-N-(3-(4-fluoropiperidin-1-yl)propyl)-6-methoxybenzo[d]imidazo[2,1-b]thiazole-7-carboxamide dihydrochloride was prepared from tert-butyl (cis)-2-(3-fluoro-4-(7-((3-(4-fluoropiperidin-1-yl)propyl)carbamoyl)-6-methoxybenzo[d]imidazo[2,1-b]thiazol-2-yl)phenyl)-4-hydroxypyrrolidine-1-carboxylate (30 mg, 0.045 mmol), following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride and was isolated as an off-white solid.

Yield 5 mg (17%). ¹H NMR (400 MHz, DMSO) δ 10.89 (br s, 1H), 10.45 (br s, 1H), 9.18 (br s, 1H), 8.85 (d, J=3.6 Hz, 1H), 8.56-8.46 (m, 1H), 8.37 (s, 1H), 8.21 (t, J=5.6 Hz, 1H), 8.13 (s, 1H), 7.61 (d, J=12.4 Hz, 1H), 7.47 (d, J=8.0 Hz, 1H), 5.17-4.81 (m, 1H), 4.83-4.66 (m, 1H), 4.64-4.45 (m, 1H), 4.04 (s, 3H), 3.51-3.45 (m, 5H), 3.24-2.94 (m, 5H), 2.78-2.64 (m, 1H), 2.34-1.88 (m, 7H). OH proton not observed. ¹⁹F NMR (376 MHz, DMSO) δ −112.95, −175.53, −186.58. m/z: [ESI⁺] 570 (M+H)⁺. (C₂₉H₃₅Cl₂F₂N₅O₃S).

### Synthesis of 2-(2-fluoro-4-((2S,4S)-4-hydroxypyrrolidin-2-yl)phenyl)-6-methoxy-N-(1-methylpiperidin-4-yl)benzo[d]imidazo[2,1-b]thiazole-7-carboxamide (Compound 203S)

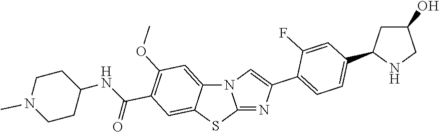 [SMILES: COc1cc2c(cc1C(=O)NC1CCN(C)CC1)sc1nc(-c3ccc([C@H]4C[C@@H](O)CN4)cc3F)cn12]

Compound 2-(2-fluoro-4-((cis)-4-hydroxypyrrolidin-2-yl)phenyl)-6-methoxy-N-(1-methylpiperidin-4-yl)benzo[d]imidazo[2,1-b]thiazole-7-carboxamide dihydrochloride was prepared from tert-butyl (cis)-2-(3-fluoro-4-(6-methoxy-7-((1-methylpiperidin-4-yl)carbamoyl)benzo[d]imidazo[2,1-b]thiazol-2-yl)phenyl)-4-hydroxypyrrolidine-1-carboxylate (30 mg, 0.048 mmol), following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride and was isolated as an off-white solid.

Yield 5 mg (17%). ¹H NMR (400 MHz, DMSO) δ 10.42 (br s, 2H), 9.17 (br s, 1H), 8.84 (t, J=3.6 Hz, 1H), 8.37-8.27 (m, 1H), 8.27-8.17 (m, 2H), 8.15-8.06 (m, 1H), 7.67-7.51 (m, 1H), 7.52-7.38 (m, 1H), 4.71 (s, 1H), 4.56 (s, 1H), 4.08-3.93 (m, 4H), 3.40-3.35 (m, 2H), 3.23-3.00 (m, 4H), 2.83-2.61 (m, 4H), 2.17-1.97 (m, 3H), 1.97-1.78 (m, 2H). OH proton not observed. ¹⁹F NMR (376 MHz, DMSO) δ −113.02. m/z: [ESI⁺]524 (M+H)⁺. (C₂₇H₃₂Cl₂FN₅O₃S).

### Synthesis of 2-(2-fluoro-4-((2S,4R)-4-hydroxypyrrolidin-2-yl)phenyl)-N-(3-(4-fluoropiperidin-1-yl)propyl)-6-methoxybenzo[d]imidazo[2,1-b]thiazole-7-carboxamide (Compound 200R)

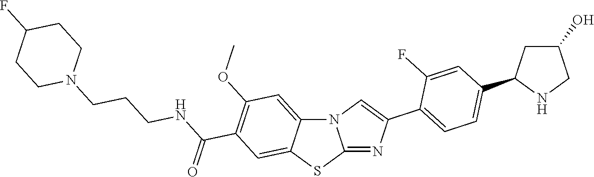 [SMILES: COc1cc2c(cc1C(=O)NCCCN1CCC(F)CC1)sc1nc(-c3ccc([C@H]4C[C@H](O)CN4)cc3F)cn12]

Compound 2-(2-fluoro-4-((trans)-4-hydroxypyrrolidin-2-yl)phenyl)-N-(3-(4-fluoropiperidin-1-yl)propyl)-6-methoxybenzo[d]imidazo[2,1-b]thiazole-7-carboxamide dihydrochloride was prepared from tert-butyl (trans)-2-(3-fluoro-4-(7-((3-(4-fluoropiperidin-1-yl)propyl)carbamoyl)-6-methoxybenzo[d]imidazo[2,1-b]thiazol-2-yl)phenyl)-4-hydroxypyrrolidine-1-carboxylate (100 mg, 0.149 mmol), following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride and was isolated as a white solid.

Yield 12 mg (13%). ¹H NMR (400 MHz, DMSO) δ 11.04 (br s, 1H), 10.57 (br s, 1H), 9.37 (br s, 1H), 8.96-8.76 (m, 1H), 8.51 (t, J=5.6 Hz, 1H), 8.36 (s, 1H), 8.21-8.07 (m, 2H), 7.66 (d, J=12.4 Hz, 1H), 7.50 (d, J=8.0 Hz, 1H), 5.23-4.64 (m, 2H), 4.61-4.50 (m, 1H), 4.04 (s, 3H), 3.68-3.34 (m, 5H), 3.28-2.96 (m, 5H), 2.38-1.95 (m, 8H). OH proton not observed. ¹⁹F NMR (376 MHz, DMSO) δ −113.02. Aliphatic ¹⁹F not observed. m/z: [ESI⁺] 570 (M+H)⁺. (C₂₉H₃₅Cl₂F₂N₅O₃S).

### Synthesis of 2-(2-fluoro-4-((2S,4R)-4-hydroxypyrrolidin-2-yl)phenyl)-6-methoxy-N-(1-methylpiperidin-4-yl)benzo[d]imidazo[2,1-b]thiazole-7-carboxamide (Compound 203R)

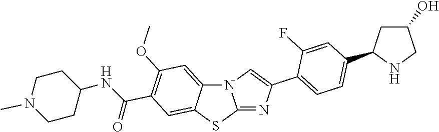 [SMILES: COc1cc2c(cc1C(=O)NC1CCN(C)CC1)sc1nc(-c3ccc([C@H]4C[C@H](O)CN4)cc3F)cn12]

Compound 2-(2-fluoro-4-((trans)-4-hydroxypyrrolidin-2-yl)phenyl)-6-methoxy-N-(1-methylpiperidin-4-yl)benzo[d]imidazo[2,1-b]thiazole-7-carboxamide dihydrochloride was prepared from tert-butyl (trans)-2-(3-fluoro-4-(6-methoxy-7-((1-methylpiperidin-4-yl)carbamoyl)benzo[d]imidazo[2,1-b]thiazol-2-yl)phenyl)-4-hydroxypyrrolidine-1-carboxylate (50 mg, 0.080 mmol), following a similar procedure to that described for the synthesis of (S)-2-(2-fluoro-4-(pyrrolidin-2-yl)phenyl)-N-(3-(4-fluoropiperidin-1-yl)propyl)-9-methyl-9H-benzo[d]imidazo[1,2-a]imidazole-7-carboxamide dihydrochloride and was isolated as a yellow green solid.

Yield 8 mg (17%). ¹H NMR (400 MHz, DMSO) δ 10.84 (br s, 1H), 10.49 (br s, 1H), 9.29 (br s, 1H), 8.86 (d, J=3.6 Hz, 1H), 8.38-8.29 (m, 1H), 8.26-8.15 (m, 2H), 8.11 (s, 1H), 7.66 (d, J=12.4 Hz, 1H), 7.50 (d, J=8.0 Hz, 1H), 4.89-4.77 (m, 1H), 4.60-4.52 (m, 1H), 4.00-3.95 (m, 4H), 3.65-3.53 (m, 1H), 3.46-3.24 (m, 2H), 3.22-3.02 (m, 3H), 2.71 (s, 3H), 2.36-2.19 (m, 2H), 2.14-1.80 (m, 4H). OH proton not observed. ¹⁹F NMR (376 MHz, DMSO) δ −112.97. m/z: [ESI⁺] 524 (M+H)⁺. (C₂₇H₃₂Cl₂FN₅O₃S).

### Example 4

### Biological Activity of Compounds of the Invention

The biological activity results of compounds of the invention are summarized in Table 2.

TABLE 2
Cellular −LogEC₅₀ values of compounds of
the invention in the immunofluorescence assay.

| | Myc Efficacy |
|---|---|
| Compound No. | (−LogEC₅₀) |
| 102R | +++ |
| 102S | +++ |
| 103R | +++ |
| 103S | +++ |
| 121R | +++ |
| 121S | +++ |
| 122R | ++ |
| 122S | ++ |
| 137R | ++ |
| 137S | ++ |
| 138 | +++ |
| 139 | ++ |
| 140 | ++ |
| 141 | ++ |
| 142 | ++ |
| 143 | + |
| 144 | +++ |
| 145 | ++ |
| 146 | ++ |
| 147 | ++ |
| 148 | + |
| 149R | +++ |
| 149S | +++ |
| 150 | +++ |
| 151 | +++ |
| 152 | + |
| 153R | +++ |
| 153S | +++ |
| 154 | + |
| 155 | +++ |
| 156 | ++ |
| 157 | ++ |
| 158 | ++ |
| 159 | +++ |
| 160 | +++ |
| 161 | +++ |
| 162 | ++ |
| 163 | ++ |
| 164 | +++ |
| 165 | +++ |
| 166 | +++ |
| 167 | ++ |
| 168 | +++ |
| 169 | +++ |
| 170 | ++ |
| 171 | + |
| 172 | ++ |
| 173 | + |
| 174 | + |
| 175 | +++ |
| 176 | +++ |
| 177 | ++ |
| 178 | +++ |
| 179 | ++ |
| 180 | ++ |
| 181 | ++ |
| 182 | +++ |
| 183 | +++ |
| 184 | +++ |
| 185 | +++ |
| 186 | +++ |
| 187 | +++ |
| 188 | +++ |
| 189 | +++ |
| 190 | +++ |
| 191 | +++ |
| 192 | +++ |
| 195 | +++ |
| 196 | +++ |
| 197 | +++ |
| 198 | +++ |
| 199 | ++ |
| 200R | +++ |
| 200S | ++ |
| 201R | ++ |
| 201S | ++ |
| 202R | ++ |
| 202S | ++ |
| 203R | +++ |
| 203S | +++ |
| 204 | ++ |
| 205 | + |
| 300 | +++ |
| 301 | +++ |
| 302 | +++ |
| 303 | +++ |
| 400R | + |
| 400S | + |
| 401 | + |
| 402 | + |
| 403 | + |
| 404 | + |
| 408 | + |
| 409 | + |
| 410 | + |

Activity (−LogEC₅₀):
− ≤3
+ >3 and <5
++ ≥5 and <6
+++ ≥6

Compounds activity is tested in tumor cell lines expressing c-Myc by using high content image analysis. c-Myc mRNA rate of translation is assayed using PSM assay, c-Myc protein levels and intracellular localization are assayed by immunofluorescence using a c-Myc specific antibody and c-Myc mRNA levels and intracellular localization is tested using specific fluorescent probes, as detailed in the Experimental Methods below (Example 6). The di-tRNA translation rate measurement specificity to c-Myc is shown by co-transfecting c-Myc specific siRNA. Transfection of labelled di-tRNA with c-Myc specific siRNA reduce the FRET signal originating from ribosome translating c-Myc, relative to cells transfected with nonrelevant siRNA.

A549 cells are incubated with active compounds and metabolically labelled with fluorescent methionine for a 4 hour pulse (click-chemistry modified methionine). Cells are fixed and newly synthesized proteins detected by using click-chemistry with a fluorescent detector.

A549 cells are incubated with compounds for 24 hours and c-Myc protein is detected by immunofluorescence. In parallel, A549 cells are incubated with compounds for 4 hours and c-Myc mRNA is visualized by microscopy using c-Myc mRNA specific fluorescent-tagged probes.

A549 human non-small cell lung carcinoma cells are treated for 24 hours with increasing compound concentration, cells are fixed and stained with a nuclei stain (DAPI) and anti-c-Myc fluorescent antibody. The c-Myc signal is quantified by image analysis, and data is exported and analyzed using TIBCO Spotfire® (TIBCO Corporation). Dose response curves are generated and fitted with logaristic regression to calculate potency (EC₅₀ values).

### Example 5

### In Vivo Activity of Compounds of the Invention

A549 xenograft model in nude mice.

NMRI nude female mice of 6-8 weeks of age are acclimated after shipping for >4 days. A549 cells, 5×10⁶ in 100 ul Matrigel:PBS (50:50), are subcutaneously injected into flanks of mice. When the tumor size reaches 80 to 200 mm³, mice are grouped with similar average tumor size in each group, 10 animals per group. Compounds are dissolved in 10% DMSO, 10% Solutol, 80% water. Compounds are given i.p. for 49 days at 3 mg/kg twice a week. Caliper measurement of tumor size are done twice a week.

### Example 6

### Experimental Methods

High Content Screen for the Identification of c-Myc Modulators

Compound effect on translation of c-Myc in A549, human non-small cell lung carcinoma cell line, is conducted using specific PSM assay using tRNAgln and tRNAser isoacceptors, as described below. A library of diverse small molecules, 90,000 compounds, is used at a final concentration of 30 μM. Image and data analyses are conducted using Anima's proprietary algorithms. False positive and toxic compounds are eliminated. Compounds are identified as hits, compounds which increase or decrease the FRET signal generated by ribosomes during c-Myc translation.

Positive hits are re-screened in the specific PSM assay, using tRNAgln and tRNAser. Hits are scored using Anima's proprietary algorithms, and compounds, which selectively inhibit c-Myc synthesis in specific PSM assay, are selected as confirmed hits. These compounds are purchased as powder to confirm activity. Re-purchased hits are tested in the specific PSM assay (tRNAgln-tRNAser) and anti-c-Myc immunofluorescence, and in counter assays to eliminate global translation modulators: (1) bulk tRNA and (2) metabolic labeling using Click-IT™ AHA (L-Azidohomoalanine).

### Cell Culture

A549 cells (ATCC® CCL-185™) are maintained in DMEM low glucose medium (Biological Industries, Cat. 01-050-1A), containing 10% fetal bovine serum, 1% L-glutamine and 1% penicillin-streptomycin solution.

SK-N-F1 cells (ATCC® CRL-2142™) are maintained in DMEM high glucose medium (Biological Industries, Cat. 01-055-1A), containing 10% fetal bovine serum, 2% L-glutamine, 1% penicillin-streptomycin solution, 1% sodium pyruvate and 1% non-essential amino acids.

Specific tRNA (tRNA isoacceptor) isolation and labeling

The specific tRNAgln (TTG) and tRNAser (CGA) are isolated for from baker's yeast (Roche) using biotinylated oligos complimentary to sequences encompassing the D-loop and anti-codon. The biotinylated oligos are mixed with total yeast tRNA and heat up to 82° C. for 10 min, followed by addition of TMA buffer (20 mM Tris, pH 7.6, 1.8M tetramethylammonium chloride, 0.2 mM EDTA). The mixture is incubated at 68° C. for 10 min, and annealed by slow cooling to 37° C. tRNA:DNA oligo mixture then is incubated with streptavidin linked agarose beads at room temperature for 30 min while shaking. Unbound tRNA and tRNA:DNA complexes are removed by centrifugation and beads are washed with 10 mM Tris-HCl (pH 7.6). The target tRNA is eluted from the resin by incubation at 45° C. or 55° C. for 7 min followed by centrifugation and collection of the supernatant to clean tubes.

The purity of the isolated tRNA isoacceptors is confirmed using fluorescent polarization assay. Purified tRNA is annealed to a complementary oligo tagged at the 3′-end with Cy3. The annealed purified tRNA isoacceptor FP signal is compared to the signal derived from annealing of a tRNA isoacceptor oligo annealed to the same Cy3-oligo. Samples with more than 80% purity are selected for labeling.

The dihydrouridines of the target tRNAs or total yeast tRNA are labeled as described in U.S. Pat. No. 8,785,119. Labeled tRNAs are purified by reverse phase HPLC and eluted with an ethanol gradient.

### Protein Synthesis Monitoring (PSM) Assays

Cy3 and Cy5 Labeled tRNA, bulk or specific, are transfected with 0.4 μl HiPerFect (Qiagen) per 384 well. First, HiPerFect is mixed with DMEM and incubated for 5 min; next, 6 nanograms Cy3-labeled tRNAgln and 6 ng Cy5-labeled tRNAser (or 9 ng each Cy3 and Cy5-labelled bulk tRNA) are diluted in 1×PBS and then added to the HiPerFect:DMEM cocktail and incubated at room temperature for 10 min. The transfection mixture is dispersed automatically into 384-well black plates. Cells are then seeded at 3,500 cells per well in complete culture medium and incubated at 37° C., 5% CO₂. Forty-eight hours after transfection compounds are added at a final concentration of 30 μM. Four hours post-treatment, cells are fixed with 4% paraformaldehyde and images are captured with Operetta microscope (Perkin Elmer) using ×20 high NA objective lens.

### Metabolic Labeling Assay

A549 cells are seeded at 3,200 cells per well in complete culture medium. Plates are incubated at 37° C., 5% CO₂ overnight. After 48 hours of incubation, the growth medium is aspirated, and cells are washed three times with HBSS. Metabolic labeling medium DMEM (-Cys-Met), containing 10% dialyzed FBS, 1% pencillin-streptomycin and 1% L-glutamine is added to the cells for 30 min. Then medium is replaced by metabolic labeling medium containing 25 μM L-Azidohomoalanine (AHA, ThermoFisher) and tested compounds at a final concentration of 30 μM, and cells are incubated for 4 hours at 37° C., 5% CO₂. Cells are washed by HBSS at 37° C. for 15 min before fixing with 4% paraformaldehyde. Cells are washed twice with 3% BSA in PBS before permeabilization with 0.5% Triton X-100 in PBS for 20 min. The AHA staining with Alexa Fluor™ 555 alkyne is performed according to the manufacturer protocol. Images are captured with Operetta microscope (Perkin Elmer) using ×20 high NA objective lens.

c-Myc Immunofluorescence Assay

A549 cells are grown in 384-wells plates (Perkin Elmer, Cat. 6057300) for 48 hours, treated with compounds and then fixed for 20 min in 4% paraformaldehyde. After that permeabilization is done using 0.1% Triton X-100 in PBS for 20 min. Primary anti-c-Myc antibody (Abcam, ab32072) staining is performed for 90 min at room temperature. Cells then are washed twice with PBS and incubated with secondary antibody (Abcam, ab150075) for 90 min at room temperature. Nuclei are stained with DAPI for 10 min and washed twice with PBS.

Cell images are taken with Operetta (Perkin Elmer, USA), a wide-field fluorescence microscope at 20× magnification. After acquisition, the images are transferred to Columbus software (Perkin-Elmer) for image analysis. In Columbus, cells are identified by their nucleus, using the “Find Nuceli” module and cytoplasm is detected based on the secondary antibody channel. Subsequently, the fluorescent signal is enumerated in the identified cell region. Then data is exported to a data analysis and visualization software, Tibco Spotfire, USA.

### Fluorescent In Situ Hybridization (FISH) Assay

A549 cells are grown in 384-wells plates (Perkin Elmer, Cat. 6057300) for 48 hours, treated with compounds for 4 hours and then fixed for 20 min in 4% paraformaldehyde. Next day, permeabilization is done for 90 min at 4° C., using 70% ethanol. Then, the cells are incubated for 10 min with 10% formamide in 10% saline-sodium citrate. Fluorescently labeled custome DNA probes that target c-Myc (Cy3, BioSearch Technologies, Cat. SMF-1063-5) and GAPDH (Cy5, BioSearch Technologies, Cat. SMF-2019-1) mRNAs are hybridized overnight at 37° C. in a dark chamber in 10% formamide. The next day, cells are washed twice with 10% formamide for 30 min. Next, nuclei are counterstained with DAPI (SIGMA, Cat. 5MG-D9542) and then cells are washed twice with PBS. FISH experiments are performed according to the probes manufacturer's protocol for adherent cells.

Following RNA FISH experiments, images of cells are taken with Operetta (Perkin Elmer, USA), a wide-field fluorescence microscope at ×63 magnification. After acquisition, the images are transferred to Columbus software for image analysis. In Columbus, cells are identified by their nucleus, using the “Find Nuceli” module, cytoplasm is detected based on the FISH-channel, and single mRNAs in the cytoplasm and transcription sites in the nucleus are detected using “Find Spots” module. Subsequently, fluorescent signals are collected for each channel in the identified regions: nucleus, cytoplasm and spots. Data is exported to a data analysis and visualization software, Tibco Spotfire, USA.

### A549 Xenograft Model in Nude Mice.

NMRI nude female mice of 6-8 weeks of age are acclimated after shipping for >4 days. A549 cells, 5×10⁶ in 100 ul Matrigel:PBS (50:50), are subcutaneously injected into flanks of mice. When the tumor size reached 80 to 200 mm³, mice are grouped with similar average tumor size in each group, 10 animals per group. Compounds are dissolved in 10% DMSO, 10% Solutol, 80% water. Compounds are given i.p. for 49 days at 3 mg/kg twice a week. Caliper measurement of tumor size is done twice a week.

1. A compound represented by the structure of formula I(k): 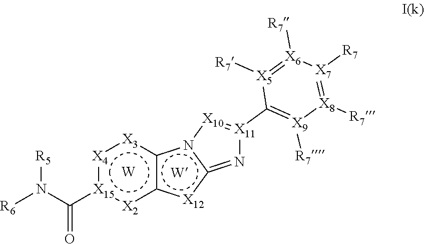
wherein
Ring W may be either aromatic or non-aromatic ring,
wherein if ring W is aromatic then
X₂, X₃, and X₄, are each independently CH, C(R) or N (e.g., C(CH₃), C(O—CH₂-cyclopropyl), C(O—CH₂-methylcyclobutyl), C(O—CH₂-3-methyloxetane), C(NH—CH₂-cyclopropyl), C(isopropoxy), C(O—CH(CH₃)—CH₂—O—CH₃), C(CH₂CH₃), C-iPr, C—CH₂-cyclopropyl, C(OCH₃), C(OCH₂CH₃), C(O—(CH₂)₂—O—CH₃, C(OCHF₂), C(Cl), C(C(O)CH₃), C(O—CH₂CH₂—O—CH₃), C(OH));
X₁₅ is C; and
wherein if ring W is non-aromatic then
X₂, X₃, and X₄, are each independently CH₂, CH(R), C(R)₂, NH, N(R), O, S, S═O or SO₂ (e.g., CH₂);
X₁₅ is CH, C(R) or N (e.g., CH, N);
X₅, X₆, X₇, X₈ and X₉ are each independently nitrogen or carbon atoms;
wherein if either one of X₅, X₆, X₇, X₈ and X₉ is nitrogen, then the respective R₇′, R₇″, R₇, R₇′″, and R₇″″ substitution is absent;
X₁₀ is N, CH, C(R), or C═O; wherein if X₁₀ is C═O then X₁₁ is N (e.g., C(CH₂—OH), C(CH₂—CH₂—OH), C(NH—CH₂-cyclopropyl), C(COOH), C(CH₃), C(cyclopropyl), C(isopropoxy));
X₁₁ is N or C; wherein if X₁₁ is N then X₁₀ is C═O;
Ring W′ may be either aromatic or non-aromatic ring,
wherein if ring W′ is aromatic then
X₁₂ is S, SO₂, O, NH, N(R), N—OH, CH═CH, CH═CH(R), C(R)═CH, N═CH, N═C(R), CH═N or C(R)═N (e.g., N—CH₂—COOH, N—CH₂—CH₂—OH, N—CH₃, N—CH₂CH₃, N-iPr, N-cyclopropyl, N—CH2-cyclopropyl);
wherein if ring W′ is non-aromatic then
X₁₂ is CH═CH, CH═CH(R), C(R)═CH, OCH₂, SCH₂, CH═N, C(R)═N, N═CH, N═C(R);
wherein if both Ring W and Ring W′ are aromatic, then at least one of X₂, X₃, and X₄ is C(R); X₁₁ is N; or X₁₂ is not S;
R₅ is H or C₁-C₅ linear or branched alkyl (e.g. methyl);
R₆ is H, F, Cl, Br, I, OH, SH, R₈—OH, R₈—SH, —R₈—O—R₁₀ (e.g., CH₂—O—CH₃, (CH₂)₂—O—CH₃ (CH₂)₃O—CH₃, (CH₂)₂—O—CH(CH₃)₂), R₈—S—R₁₀ (e.g., (CH₂)₃—S—(CH₂)₂CH₃), R₈-NHC(O)—R₁₀, —O—R₈-R₁₀, R₈-(substituted or unsubstituted C₃-C₈ cycloalkyl) (e.g., CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted, saturated, unsaturated or aromatic, single, fused or spiro 3-10 membered heterocyclic ring) (e.g., (CH₂)₃-piperidine, (CH₂)₃-4-fluoro-piperidine, (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-oxa-azaspirodecane, (CH₂)₃-dimethylpyrazole, CH₂-2-oxo-methylpyrrolidine, CH₂-methyl-azetidine, CH₂-azaspiroheptane), CF₃, CD₃, OCD₃, CN, NO₂, —CH₂CN, —R₈CN, NH₂, NHR, N(R)₂, NH(R₁₀), N(R₁₀)(R₁₁), R₈-N(R₁₀)(R₁₁) (e.g., (CH₂)₂—NH₂, (CH₂)₃—N(CH₂CH₃)₂, (CH₂)₃—N(CH(CH₃)₂)₂, (CH₂)₃-piperidine, (CH₂)₃-4-fluoro-piperidine, (CH₂)₃-4-cyano-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃)), R₉—R₈-N(R₁₀)(R₁₁) (e.g., (CH₂)₂—C(O)-piperidine), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀ (e.g., C(O)CH₃), C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₅ cycloalkyl (e.g., cyclopropyl, cyclobutyl, cyclohexyl, methoxycyclopropyl, methylcyclobutyl, aminomethyl-cyclobutyl, methoxycyclobutyl, 2,3-dihydro-1H-indenol), R₈-(substituted or unsubstituted C₃-C₈ cycloalkyl), substituted or unsubstituted 3-8 membered heterocyclic ring (e.g., piperidine, 1-methyl-piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), substituted or unsubstituted benzyl;
or R₆ and R₅ are joined to form a substituted or unsubstituted 5-8 membered heterocyclic ring (e.g., azepane, piperazine, 2-(piperazin-1-yl)acetamide;
or R₆ is represented by the structure of formula B or Bi: 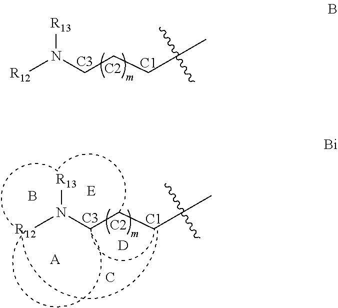
wherein
m is 0 or 1; and
R₁₂ is R₂₀ or C₁-C₅ C(O)-alkyl, and R₁₃ is R₃₀; or
R₁₂ and R₁₃ are both H;
R₁₂ and R₁₃ are each independently H or substituted or unsubstituted C₁-C₅ alkyl (e.g., ethyl, trifluoroethyl);
R₁₂ and C3 are joined to form ring A and R₁₃ is R₃₀; or
R₁₂ and R₁₃ are joined to form ring B; or
R₁₂ and C1 are joined to form ring C and R₁₃ is R₃₀; or
C1 and C3 are joined to form ring D and R₁₂ and R₁₃ are each independently R₃₀; or
R₁₃ and C2 are joined to form ring E, m is 1, and R₁₂ is R₃₀; or
R₁₂ and R₁₃ are joined to form ring B and C1 and C3 are joined to form ring D;
wherein
Ring A, C and E are each independently a substituted or unsubstituted single spiro or fused 3-8 membered heterocyclic ring (e.g., A: pyrrolidine, methylpyrrolidine, ethylpyrrolidine); C: piperidine, pyrrolidine, methyl-2-oxopyrrolidine, pyran-pyrrolidine, methyl-azetidine, azabicyclooctane, 2-azabicyclo[2.1.1]hexane, 2-azaspiro[3.3]heptane; E: pyrrolidine, azetidine, ethylpyrrolidine, oxopyrrolidine, methylpiperidine;
Ring B is a substituted or unsubstituted single, spiro or fused 3-8 membered heterocyclic ring (B: piperidine, piperidin-2-one, 4-fluoropiperidin-2-one, piperidine-4-carbonitrile, 4-fluoropiperidine, 4-fluoro-2-methylpiperidine, methyl-piperidin, fluoropiperidine, difluoropiperidine, pyrrolidine, piperazine, methylpyrrolidine, thiomorpholine 1,1-dioxide, 2-oxa-6-azaspiro[3.3]heptane, methyl-piperazine, dimethyl-pyrazole, imidazole, 2-methyl-2,5-diazabicyclo[2.2.1]heptane, hydroxymethyl-pyrrolidine, diazabicyclo[2.2.1]heptane, 6-fluoro-3-azabicyclo[3.1.1]heptane; and
Ring D is a substituted or unsubstituted C₃-C₈ cycloalkyl (e.g., cyclobutane, cyclohexane);
R₇ is H, F, Cl, Br, I, OH, O—R₂₀, SH, R₈—OH, R₈—SH, SR₁₀, —R₈—O—R₁₀, —R₈—S—R₁₀, R₈—(C₃-C₅ cycloalkyl), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)), C(O)N(R₁₀)(R₁₁) (e.g., C(O)NH(CH₃), C(O)NH(CH₂CH₂OCH₃), C(O)NH(CH₂CH₂OH)), SO₂R, SO₂N(R₁₀)(R₁₁), CH(CF₃)(NH—R₁₀), C₁-C₅ linear or branched, substituted or unsubstituted alkyl (e.g., methylimidazole, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy, ethoxy), optionally wherein at least one methylene group (CH₂) in the alkoxy is replaced with an oxygen atom, C₁-C₅ linear or branched thioalkyl, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopropanol, cyclohexyl), substituted or unsubstituted 4-7 membered heterocyclic ring (e.g., morpholine (e.g., 2 or 3-morpholine), tetrahydrofuran, tetrahydropyran, oxetane, oxetan-3-ol, pyrrolidine, pyrrolidine-3-ol, 1-methylpyrrolidine, pyrrolidin-2-one, pyrrolidinone, imidazole, pyrazole, piperazine, piperidine, piperidine-4-ol, piperidine-4-carbonitrile, 4-fluoropiperidine, oxadiazole, triazole, 2-oxopyrrolidine, pyridine, 1-methylpyridine), R₈-(substituted or unsubstituted single, fused or spiro 3-8 membered heterocyclic ring), substituted or unsubstituted aryl, substituted or unsubstituted benzyl;
R₇′, R₇″, R₇′″ and R₇″″ are each independently H, F, Cl, Br, I, OH, O—R₂₀, SH, R₈—OH, R₈—SH, —R₈—O—R₁₀, R₈—(C₃-C₅ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclohexyl), substituted or unsubstituted 3-8 membered heterocyclic ring (e.g., morpholine, pyran, oxetane, pyrrolidine, imidazole, piperazine, piperidine, dioxazole, 2-oxopyrrolidine), substituted or unsubstituted aryl (e.g., phenyl), substituted or unsubstituted benzyl;
or R₇′ and R₇″ are joined to form a 3-8 membered substituted or unsubstituted, saturated, unsaturated or aromatic, carbocyclic or heterocyclic ring (e.g., cyclopentyl, cyclohexyl, piperidine, tetrahydrofuran, tetrahydropyran, pyrrolidine);
or R₇″ and R₇ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
or R₇ and R₇′″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
or R₇′″ and R₇″″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);
R₂₀ is represented by the following structure: 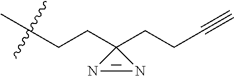 [SMILES: *CCC1(CCC#C)N=N1]
R₃₀ is H, R₂₀, F, Cl, Br, I, OH, SH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂-O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂O—CH₃), —R₈—O—R₁₀, —R₈-R₁₀ (e.g., (CH₂)₂O—CH₃), substituted or unsubstituted aryl (e.g., phenyl), substituted or unsubstituted heteroaryl (e.g., pyridine (2, 3, and 4-pyridine);
R is F, Cl, Br, I, OH, SH, COOH, CO(R₁₀), C(O)CH₃, NH(R₁₀), NH—CH₂-cyclopropyl, N(R₁₀)(R₁₁), CF₃, CN, NO₂, C₁-C₅ linear or branched, substituted or unsubstituted alkyl (e.g., methyl, ethyl, iPr, CH₂-cyclopropyl, CH₂—OH, CH₂—CH₂—OH, CH₂—CH₂—O—CH₂—CH₂—O—CH₃, CH₂—O—CH₂—CH₂—O—CH₃), C₃-C₈ substituted or unsubstituted cycloalkyl, cyclopropyl, substituted or unsubstituted C₁-C₅ linear or branched alkoxy, (e.g., methoxy, ethoxy, O—(CH₂CH₃), OCHF₂, O—(CH₂)₂—O—CH₃, isopropoxy, O—(CH₂)-cyclopropyl, O—CH₂-methylcyclobutyl, O—CH₂-3-methyloxetane, O—CH(CH₃)—CH₂—O—CH₃) C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂O—CH₃), —R₈—O—R₁₀, O—R₈-R₁₀ (e.g. O—(CH₂)₂—O—CH₃, O—(CH₂CH₃), O—(CH₂)-cyclopropyl), —R₈-R₁₀ (e.g., (CH₂)-cyclopropyl, (CH₂)₂O—CH₃, (CH₂)—OH, (CH₂)₂—OH, (CH₂)—COOH), substituted or unsubstituted aryl (e.g., phenyl), substituted or unsubstituted heteroaryl (e.g., pyridine (2, 3, and 4-pyridine);
each R₅ is independently [CH₂]ₚ
wherein p is between 1 and 10 (e.g., 1, 2);
R₉ is [CH]_{q}, [C]_{q}
wherein q is between 2 and 10;
R₁₀ and R₁₁ are each independently H, OH, COOH, C₁-C₅ substituted or unsubstituted linear or branched alkyl (e.g., methyl, ethyl, CH₂-cyclopropyl, CH₂—CH₂—O—CH₃), C₃-C₈ substituted or unsubstituted cycloalkyl (e.g., cyclopropyl), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;
or R₁₀ and R₁₁ are joined to form a substituted or unsubstituted 3-8 membered heterocyclic ring (e.g., piperazine, piperidine),
n is an integer between 0 and 4 (e.g., 1, 2);
or a compound represented by any one of the following structures:

| Compound No. | Compound Name |
|---|---|
| 130R | (R)-3-(2-fluoro-4-(pyrrolidin-2-yl)phenyl)-N-(3-(4-fluoropiperidin-1- |
| | yl)propyl)benzo[4,5]thiazolo[2,3-c][1,2,4]triazole-7-carboxamide |
| 130S | (S)-3-(2-fluoro-4-(pyrrolidin-2-yl)phenyl)-N-(3-(4-fluoropiperidin-1- |
| | yl)propyl)benzo[4,5]thiazolo[2,3-c][1,2,4]triazole-7-carboxamide |
| 205 | N-(3-(4-fluoropiperidin-1-yl)propyl)-6-methoxy-2-(pyrrolidin-2- |
| | yl)benzo[d]imidazo[2,1-b]thiazole-7-carboxamide |
| 400S | (S)-2-(2-fluoro-4-(pyrrolidin-2-yl)phenyl)-N-(3-(4-fluoropiperidin-1- |
| | yl)propyl)-9H-benzo[d]imidazo[1,2-a]imidazole-6-carboxamide |
| 400R | (R)-2-(2-fluoro-4-(pyrrolidin-2-yl)phenyl)-N-(3-(4-fluoropiperidin-1- |
| | yl)propyl)-9H-benzo[d]imidazo[1,2-a]imidazole-6-carboxamide |
| 401 | 7-bromo-2-(2-fluoro-4-(pyrrolidin-2-yl)phenyl)-6- |
| | isopropoxybenzo[d]imidazo[2,1-b]thiazole |
| 402 | 2-(2-fluoro-4-(pyrrolidin-2-yl)phenyl)-5,6,7,8- |
| | tetrahydrobenzo[d]imidazo[2,1-b]thiazole-7-carboxylic acid |
| 403 | 2-(2-fluoro-4-(pyrrolidin-2-yl)phenyl)-6-methoxybenzo[d]imidazo[2,1- |
| | b]thiazole-7-carboxylic acid |
| 404 | 2-bromo-N-(3-(4-fluoropiperidin-1-yl)propyl)-6- |
| | methoxybenzo[d]imidazo[2,1-b]thiazole-7-carboxamide |
| 408 | N-(3-(4-fluoropiperidin-1-yl)propyl)-2-(pyrrolidin-2- |
| | ylethynyl)benzo[d]imidazo[2,1-b]thiazole-7-carboxamide |
| 409 | N-(3-(4-fluoropiperidin-1-yl)propyl)-2-(pyrrolidine-2- |
| | carboxamido)benzo[d]imidazo[2,1-b]thiazole-7-carboxamide |
| 410 | N-(3-(4-fluoropiperidin-1-yl)propyl)-6-methoxy-2-(pyrrolidine-2- |
| | carboxamido)benzo[d]imidazo[2,1-b]thiazole-7-carboxamide |

or its pharmaceutically acceptable salt, stereoisomer, tautomer, hydrate, N-oxide, reverse amide analog, isotopic variant (e.g., deuterated analog), or any combination thereof.

2. The compound of claim 1,
wherein R₅ is H or CH₃;
wherein R is H, C₁-C₅ linear or branched, substituted or unsubstituted alkyl (preferably methyl), substituted or unsubstituted 3-8 membered heterocyclic ring (preferably substituted or unsubstituted piperidine or tetrahydropyran), R₈—N(R₁₀)(R₁₁) (preferably (CH₂)₂—NH₂, (CH₂)₃—NH₂, (CH₂)₃-piperidine or (CH₂)₃-4-fluoro-piperidine), or R₆ is represented by the structure of formula Bi;
wherein R₇ is H, C(O)N(R₁₀)(R₁₁) (preferably C(O)NH(CH₃)), C₁-C₅ linear or branched, substituted or unsubstituted alkyl (preferably methyl), substituted or unsubstituted C₃-C₈ cycloalkyl (preferably cyclopropanol), substituted or unsubstituted 4-7 membered heterocyclic ring (preferably morpholine, pyrrolidine, pyrrolidine-3-ol, piperidine, piperidine-4-ol, tetrahydrofuran, oxetane, oxetan-3-ol, pyridine, or 1-methylpyridine), or substituted or unsubstituted aryl;
wherein R₇′ is H or F;
wherein Ring W is aromatic and one of X₂, X₃ and X₄ is C(R); preferably wherein R is alkoxy, more preferably methoxy;
wherein X₁₂ is O, NH, N(R), CH═N, N═CH, CH═CH, OCH₂, or N—OH: preferably wherein R is C₁-C₅ linear or branched, substituted or unsubstituted alkyl, substituted or unsubstituted C₁-C₅ linear or branched alkoxy, O—R₈-R₁₀, OH, NH(R₁) haloalkoxy or cycloalkyl;
wherein R is CH₂—COOH, CH₂—CH₂—OH, CH₃, CH₂CH₃, iPr, cyclopropyl or CH₂-cyclopropyl;
wherein X₄ is C(R) and R is alkoxy, preferably methoxy;
or any combination thereof.

3. (canceled)

4. The compound of claim 2, wherein R₁₂ and R₁₃ of formula Bi are joined to form a substituted or unsubstituted piperidine ring, or wherein R₁₀ and R₁₁ are joined to form a substituted or unsubstituted 3-8 membered heterocyclic ring (e.g., piperidine or 4-fluoro-piperidine).

5. (canceled)

6. (canceled)

7. (canceled)

8. (canceled)

9. (canceled)

10. (canceled)

11. The compound of claim 1, selected from the following:

| Compound No. | Compound Name |
|---|---|
| 100R | (R)-2-(2-fluoro-4-(tetrahydrofuran-2-yl)phenyl)-6-((3-methyloxetan-3-yl)oxy)-N- |
| | (3-(piperidin-1-yl)propyl)benzo[d]imidazo[2,1-b]thiazole-7-carboxamide |
| 100S | (S)-2-(2-fluoro-4-(tetrahydrofuran-2-yl)phenyl)-6-((3-methyloxetan-3-yl)oxy)-N-(3- |
| | (piperidin-1-yl)propyl)benzo[d]imidazo[2,1-b]thiazole-7-carboxamide |
| 101R | 6-(((R)-1-methoxypropan-2-yl)oxy)-N-(3-(piperidin-1-yl)propyl)-2-(4- |
| | (tetrahydrofuran-2-yl)phenyl)benzo[d]imidazo[2,1-b]thiazole-7-carboxamide |
| 101S | 6-(((S)-1-methoxypropan-2-yl)oxy)-N-(3-(piperidin-1-yl)propyl)-2-(4- |
| | (tetrahydrofuran-2-yl)phenyl)benzo[d]imidazo[2,1-b]thiazole-7-carboxamide |
| 102S | (S)-2-(2-fluoro-4-(pyrrolidin-2-yl)phenyl)-N-(3-(4-fluoropiperidin-1-yl)propyl)-6- |
| | methylbenzo[d]imidazo[2,1-b]thiazole-7-carboxamide |
| 102R | (R)-2-(2-fluoro-4-(pyrrolidin-2-yl)phenyl)-N-(3-(4-fluoropiperidin-1-yl)propyl)-6- |
| | methylbenzo[d]imidazo[2,1-b]thiazole-7-carboxamide |
| 104S | (S)-2-(2-fluoro-4-(pyrrolidin-2-yl)phenyl)-6-((3-methyloxetan-3-yl)oxy)-N-(3- |
| | (piperidin-1-yl)propyl)benzo[d]imidazo[2,1-b]thiazole-7-carboxamide |
| 104R | (R)-2-(2-fluoro-4-(pyrrolidin-2-yl)phenyl)-6-((3-methyloxetan-3-yl)oxy)-N-(3- |
| | (piperidin-1-yl)propyl)benzo[d]imidazo[2,1-b]thiazole-7-carboxamide |
| 105R | 6-(((R)-1-methoxypropan-2-yl)oxy)-N-(3-(piperidin-1-yl)propyl)-2-(4-(pyrrolidin- |
| | 2-yl)phenyl)benzo[d]imidazo[2,1-b]thiazole-7-carboxamide |
| 105S | 6-(((S)-1-methoxypropan-2-yl)oxy)-N-(3-(piperidin-1-yl)propyl)-2-(4-(pyrrolidin-2- |
| | yl)phenyl)benzo[d]imidazo[2,1-b]thiazole-7-carboxamide |
| 106S | (S)-2-(2-fluoro-4-(pyrrolidin-2-yl)phenyl)-6-isopropoxy-N-(3-(piperidin-1- |
| | yl)propyl)benzo[d]imidazo[2,1-b]thiazole-7-carboxamide |
| 106R | (R)-2-(2-fluoro-4-(pyrrolidin-2-yl)phenyl)-6-isopropoxy-N-(3-(piperidin-1- |
| | yl)propyl)benzo[d]imidazo[2,1-b]thiazole-7-carboxamide |
| 107S | (S)-2-(2-fluoro-4-(pyrrolidin-2-yl)phenyl)-5-isopropoxy-N-(3-(piperidin-1- |
| | yl)propyl)benzo[d]imidazo[2,1-b]thiazole-7-carboxamide |
| 107R | (R)-2-(2-fluoro-4-(pyrrolidin-2-yl)phenyl)-5-isopropoxy-N-(3-(piperidin-1- |
| | yl)propyl)benzo[d]imidazo[2,1-b]thiazole-7-carboxamide |
| 108S | (S)-2-(2-fluoro-4-(pyrrolidin-2-yl)phenyl)-8-isopropoxy-N-(3-(piperidin-1- |
| | yl)propyl)benzo[d]imidazo[2,1-b]thiazole-7-carboxamide |
| 108R | (R)-2-(2-fluoro-4-(pyrrolidin-2-yl)phenyl)-8-isopropoxy-N-(3-(piperidin-1- |
| | yl)propyl)benzo[d]imidazo[2,1-b]thiazole-7-carboxamide |
| 110S | (S)-2-(2-fluoro-4-(tetrahydrofuran-2-yl)phenyl)-N-(3-(4-fluoropiperidin-1- |
| | yl)propyl)-6-((3-methyloxetan-3-yl)methoxy)benzo[d]imidazo[2,1-b]thiazole-7- |
| | carboxamide |
| 110R | (R)-2-(2-fluoro-4-(tetrahydrofuran-2-yl)phenyl)-N-(3-(4-fluoropiperidin-1- |
| | yl)propyl)-6-((3-methyloxetan-3-yl)methoxy)benzo[d]imidazo[2,1-b]thiazole-7- |
| | carboxamide |
| 111S | (S)-6-(cyclopropylmethoxy)-2-(2-fluoro-4-(tetrahydrofuran-2-yl)phenyl)-N-(3-(4- |
| | fluoropiperidin-1-yl)propyl)benzo[d]imidazo[2,1-b]thiazole-7-carboxamide |
| 111R | (R)-6-(cyclopropylmethoxy)-2-(2-fluoro-4-(tetrahydrofuran-2-yl)phenyl)-N-(3-(4- |
| | fluoropiperidin-1-yl)propyl)benzo[d]imidazo[2,1-b]thiazole-7-carboxamide |
| 112 | 6-(cyclopropylmethoxy)-2-(2-fluoro-4-(1-hydroxycyclopropyl)phenyl)-N-(3-(4- |
| | fluoropiperidin-1-yl)propyl)benzo[d]imidazo[2,1-b]thiazole-7-carboxamide |
| 113 | 6-(cyclopropylmethoxy)-2-(2-fluoro-4-(3-hydroxyoxetan-3-yl)phenyl)-N-(3-(4- |
| | fluoropiperidin-1-yl)propyl)benzo[d]imidazo[2,1-b]thiazole-7-carboxamide |
| 118S | (S)-6-((cyclopropylmethyl)amino)-2-(2-fluoro-4-(tetrahydrofuran-2-yl)phenyl)-N- |
| | (3-(4-fluoropiperidin-1-yl)propyl)benzo[d]imidazo[2,1-b]thiazole-7-carboxamide |
| 118R | (R)-6-((cyclopropylmethyl)amino)-2-(2-fluoro-4-(tetrahydrofuran-2-yl)phenyl)-N- |
| | (3-(4-fluoropiperidin-1-yl)propyl)benzo[d]imidazo[2,1-b]thiazole-7-carboxamide |
| 119S | (S)-5-(cyclopropylmethoxy)-2-(2-fluoro-4-(tetrahydrofuran-2-yl)phenyl)-N-(3-(4- |
| | fluoropiperidin-1-yl)propyl)benzo[d]imidazo[2,1-b]thiazole-7-carboxamide |
| 119R | (R)-5-(cyclopropylmethoxy)-2-(2-fluoro-4-(tetrahydrofuran-2-yl)phenyl)-N-(3-(4- |
| | fluoropiperidin-1-yl)propyl)benzo[d]imidazo[2,1-b]thiazole-7-carboxamide |
| 120S | (S)-8-(cyclopropylmethoxy)-2-(2-fluoro-4-(tetrahydrofuran-2-yl)phenyl)-N-(3-(4- |
| | fluoropiperidin-1-yl)propyl)benzo[d]imidazo[2,1-b]thiazole-7-carboxamide |
| 120R | (R)-8-(cyclopropylmethoxy)-2-(2-fluoro-4-(tetrahydrofuran-2-yl)phenyl)-N-(3-(4- |
| | fluoropiperidin-1-yl)propyl)benzo[d]imidazo[2,1-b]thiazole-7-carboxamide |
| 121S | (S)-2-(2-fluoro-4-(pyrrolidin-2-yl)phenyl)-N-(3-(4-fluoropiperidin-1-yl)propyl)-9- |
| | methyl-9H-benzo[d]imidazo[1,2-a]imidazole-7-carboxamide |
| 121R | (R)-2-(2-fluoro-4-(pyrrolidin-2-yl)phenyl)-N-(3-(4-fluoropiperidin-1-yl)propyl)-9- |
| | methyl-9H-benzo[d]imidazo[1,2-a]imidazole-7-carboxamide |
| 122S | (S)-2-(2-fluoro-4-(pyrrolidin-2-yl)phenyl)-N-(3-(4-fluoropiperidin-1-yl)propyl)-9H- |
| | benzo[d]imidazo[1,2-a]imidazole-7-carboxamide |
| 122R | (R)-2-(2-fluoro-4-(pyrrolidin-2-yl)phenyl)-N-(3-(4-fluoropiperidin-1-yl)propyl)- |
| | 9H-benzo[d]imidazo[1,2-a]imidazole-7-carboxamide |
| 123S | (S)-2-(2-fluoro-4-(pyrrolidin-2-yl)phenyl)-N-(3-(4-fluoropiperidin-1- |
| | yl)propyl)benzo[d]imidazo[2,1-b]oxazole-7-carboxamide |
| 123R | (R)-2-(2-fluoro-4-(pyrrolidin-2-yl)phenyl)-N-(3-(4-fluoropiperidin-1- |
| | yl)propyl)benzo[d]imidazo[2,1-b]oxazole-7-carboxamide |
| 124S | (S)-2-(2-fluoro-4-(pyrrolidin-2-yl)phenyl)-N-(3-(4-fluoropiperidin-1-yl)propyl)-9- |
| | hydroxy-9H-benzo[d]imidazo[1,2-a]imidazole-7-carboxamide |
| 124R | (R)-2-(2-fluoro-4-(pyrrolidin-2-yl)phenyl)-N-(3-(4-fluoropiperidin-1-yl)propyl)-9- |
| | hydroxy-9H-benzo[d]imidazo[1,2-a]imidazole-7-carboxamide |
| 125S | (S)-2-(2-fluoro-4-(pyrrolidin-2-yl)phenyl)-N-(3-(4-fluoropiperidin-1-yl)propyl)-9- |
| | (2-hydroxyethyl)-9H-benzo[d]imidazo[1,2-a]imidazole-7-carboxamide |
| 125R | (R)-2-(2-fluoro-4-(pyrrolidin-2-yl)phenyl)-N-(3-(4-fluoropiperidin-1-yl)propyl)-9- |
| | (2-hydroxyethyl)-9H-benzo[d]imidazo[1,2-a]imidazole-7-carboxamide |
| 126S | (S)-2-(2-(2-fluoro-4-(pyrrolidin-2-yl)phenyl)-7-((3-(4-fluoropiperidin-1- |
| | yl)propyl)carbamoyl)-9H-benzo[d]imidazo[1,2-a]imidazol-9-yl)acetic acid |
| 126R | (R)-2-(2-(2-fluoro-4-(pyrrolidin-2-yl)phenyl)-7-((3-(4-fluoropiperidin-1- |
| | yl)propyl)carbamoyl)-9H-benzo[d]imidazo[1,2-a]imidazol-9-yl)acetic acid |
| 131S | (S)-2-(2-fluoro-4-(pyrrolidin-2-yl)phenyl)-N-(3-(4-fluoropiperidin-1-yl)propyl)-3- |
| | oxo-2,3-dihydrobenzo[4,5]thiazolo[2,3-c][1,2,4]triazole-7-carboxamide |
| 131R | (R)-2-(2-fluoro-4-(pyrrolidin-2-yl)phenyl)-N-(3-(4-fluoropiperidin-1-yl)propyl)-3- |
| | oxo-2,3-dihydrobenzo[4,5]thiazolo[2,3-c][1,2,4]triazole-7-carboxamide |
| 135 | 2-(2-fluoro-4-(pyrrolidin-2-yl)phenyl)-N-(3-(4-fluoropiperidin-1- |
| | yl)propyl)imidazo[1,2-a]quinoxaline-7-carboxamide |
| 136 | 2-(2-fluoro-4-(pyrrolidin-2-yl)phenyl)-N-(3-(4-fluoropiperidin-1- |
| | yl)propyl)imidazo[1,2-a]quinazoline-7-carboxamide |
| 137S | (S)-2-(2-fluoro-4-(pyrrolidin-2-yl)phenyl)-N-(3-(4-fluoropiperidin-1-yl)propyl)-8- |
| | methylbenzo[d]imidazo[2,1-b]thiazole-7-carboxamide |
| 137R | (R)-2-(2-fluoro-4-(pyrrolidin-2-yl)phenyl)-N-(3-(4-fluoropiperidin-1-yl)propyl)-8- |
| | methylbenzo[d]imidazo[2,1-b]thiazole-7-carboxamide |
| 138 | 9-ethyl-2-(2-fluoro-4-(pyrrolidin-2-yl)phenyl)-N-(3-(4-fluoropiperidin-1- |
| | yl)propyl)-9H-benzo[d]imidazo[1,2-a]imidazole-7-carboxamide |
| 139 | 2-(2-fluoro-4-(pyrrolidin-2-yl)phenyl)-N-(3-(4-fluoropiperidin-1-yl)propyl)-9- |
| | isopropyl-9H-benzo[d]imidazo[1,2-a]imidazole-7-carboxamide |
| 140 | 9-cyclopropyl-2-(2-fluoro-4-(pyrrolidin-2-yl)phenyl)-N-(3-(4-fluoropiperidin-1- |
| | yl)propyl)-9H-benzo[d]imidazo[1,2-a]imidazole-7-carboxamide |
| 141 | 9-(cyclopropylmethyl)-2-(2-fluoro-4-(pyrrolidin-2-yl)phenyl)-N-(3-(4- |
| | fluoropiperidin-1-yl)propyl)-9H-benzo[d]imidazo[1,2-a]imidazole-7-carboxamide |
| 142 | 2-(2-fluoro-4-(pyrrolidin-2-yl)phenyl)-N-(3-(4-fluoropiperidin-1-yl)propyl)-5,8- |
| | dihydroimidazo[2′,1′:2,3]thiazolo[5,4-c]pyridine-7(6H)-carboxamide |
| 143 | 2-(2-fluoro-4-(pyrrolidin-2-yl)phenyl)-N-(3-(4-fluoropiperidin-1-yl)propyl)-6- |
| | isopropylbenzo[d]imidazo[2,1-b]thiazole-7-carboxamide |
| 144 | 2-(2-fluoro-4-(pyrrolidin-2-yl)phenyl)-N-(3-(4-fluoropiperidin-1-yl)propyl)-6- |
| | methoxybenzo[d]imidazo[2,1-b]thiazole-7-carboxamide |
| 145 | 2-(2-fluoro-4-(pyrrolidin-2-yl)phenyl)-N-(3-(4-fluoropiperidin-1-yl)propyl)-6,9- |
| | dimethyl-9H-benzo[d]imidazo[1,2-a]imidazole-7-carboxamide |
| 146 | 6-ethyl-2-(2-fluoro-4-(pyrrolidin-2-yl)phenyl)-N-(3-(4-fluoropiperidin-1- |
| | yl)propyl)benzo[d]imidazo[2,1-b]thiazole-7-carboxamide |
| 147 | 2-(2-fluoro-4-(pyrrolidin-2-yl)phenyl)-N-(3-(4-fluoropiperidin-1-yl)propyl)-6- |
| | isopropoxybenzo[d]imidazo[2,1-b]thiazole-7-carboxamide |
| 148 | 8-ethyl-2-(2-fluoro-4-(pyrrolidin-2-yl)phenyl)-N-(3-(4-fluoropiperidin-1- |
| | yl)propyl)benzo[d]imidazo[2,1-b]thiazole-7-carboxamide |
| 149S | (S)-2-(2-fluoro-4-(pyrrolidin-2-yl)phenyl)-N-(3-(4-fluoropiperidin-1-yl)propyl)-6- |
| | methoxybenzo[d]imidazo[2,1-b]thiazole-7-carboxamide |
| 149R | (R)-2-(2-fluoro-4-(pyrrolidin-2-yl)phenyl)-N-(3-(4-fluoropiperidin-1-yl)propyl)-6- |
| | methoxybenzo[d]imidazo[2,1-b]thiazole-7-carboxamide |
| 150 | N-(3-(4-fluoropiperidin-1-yl)propyl)-6-(2-methoxyethoxy)-2-(4-(pyrrolidin-2- |
| | yl)phenyl)benzo[d]imidazo[2,1-b]thiazole-7-carboxamide |
| 151 | 2-(2-fluoro-4-(5-oxopyrrolidin-2-yl)phenyl)-N-(3-(4-fluoropiperidin-1-yl)propyl)- |
| | 6-methoxybenzo[d]imidazo[2,1-b]thiazole-7-carboxamide |
| 152 | 2-(2-fluoro-4-(pyrrolidin-2-yl)phenyl)-N-(3-(4-fluoropiperidin-1-yl)propyl)-5,6,7,8- |
| | tetrahydrobenzo[d]imidazo[2,1-b]thiazole-7-carboxamide |
| 153S | 2-(2-fluoro-4-(pyrrolidin-2-yl)phenyl)-6-methoxy-N-((S)-1-methylpiperidin-3- |
| | yl)benzo[d]imidazo[2,1-b]thiazole-7-carboxamide |
| 153R | 2-(2-fluoro-4-(pyrrolidin-2-yl)phenyl)-6-methoxy-N-((R)-1-methylpiperidin-3- |
| | yl)benzo[d]imidazo[2,1-b]thiazole-7-carboxamide |
| 154 | 2-(2-fluoro-4-(pyrrolidin-2-yl)phenyl)-5,6,7,8-tetrahydrobenzo[d]imidazo[2,1- |
| | b]thiazole-7-carboxamide |
| 155 | 2-(2-fluoro-4-(pyrrolidin-2-yl)phenyl)-6-methoxybenzo[d]imidazo[2,1-b]thiazole- |
| | 7-carboxamide |
| 156 | 6-(cyclopropylmethyl)-2-(2-fluoro-4-(pyrrolidin-2-yl)phenyl)-N-(3-(4- |
| | fluoropiperidin-1-yl)propyl)benzo[d]imidazo[2,1-b]thiazole-7-carboxamide |
| 157 | N-(3-(4-fluoropiperidin-1-yl)propyl)-6-methoxy-2-(4- |
| | (methylcarbamoyl)phenyl)benzo[d]imidazo[2,1-b]thiazole-7-carboxamide |
| 158 | 8-(cyclopropylmethyl)-2-(2-fluoro-4-(pyrrolidin-2-yl)phenyl)-N-(3-(4- |
| | fluoropiperidin-1-yl)propyl)benzo[d]imidazo[2,1-b]thiazole-7-carboxamide |
| 159 | 2-(2-fluoro-4-(methylcarbamoyl)phenyl)-N-(3-(4-fluoropiperidin-1-yl)propyl)-6- |
| | methoxybenzo[d]imidazo[2,1-b]thiazole-7-carboxamide |
| 160 | 2-(2-fluoro-4-(pyrrolidin-2-yl)phenyl)-6-methoxy-N-methylbenzo[d]imidazo[2,1- |
| | b]thiazole-7-carboxamide |
| 161 | N-(3-(4-fluoropiperidin-1-yl)propyl)-6-methoxy-2-(pyridin-4- |
| | yl)benzo[d]imidazo[2,1-b]thiazole-7-carboxamide |
| 162 | N-(3-(4-fluoropiperidin-1-yl)propyl)-6-methoxy-2-(m-tolyl)benzo[d]imidazo[2,1- |
| | b]thiazole-7-carboxamide |
| 163 | 6-methoxy-N-(1-methylpiperidin-3-yl)-2-(m-tolyl)benzo[d]imidazo[2,1-b]thiazole- |
| | 7-carboxamide |
| 164 | 6-(difluoromethoxy)-N-(3-(4-fluoropiperidin-1-yl)propyl)-2-(4-(pyrrolidin-2- |
| | yl)phenyl)benzo[d]imidazo[2,1-b]thiazole-7-carboxamide |
| 165 | N-(3-(4-fluoropiperidin-1-yl)propyl)-6-methoxy-2-(2-methylpyridin-4- |
| | yl)benzo[d]imidazo[2,1-b]thiazole-7-carboxamide |
| 166 | 2-(2-fluoro-4-(pyrrolidin-2-yl)phenyl)-N-(3-(4-fluoropiperidin-1-yl)propyl)-6-(2- |
| | methoxyethoxy)benzo[d]imidazo[2,1-b]thiazole-7-carboxamide |
| 167 | 2-(2-fluoro-5-methylphenyl)-N-(3-(4-fluoropiperidin-1-yl)propyl)-6- |
| | methoxybenzo[d]imidazo[2,1-b]thiazole-7-carboxamide |
| 168 | 2-(2-fluoro-4-(methylcarbamoyl)phenyl)-6-methoxy-N-(1-methylpiperidin-3- |
| | yl)benzo[d]imidazo[2,1-b]thiazole-7-carboxamide |
| 169 | 6-methoxy-N-(1-methylpiperidin-3-yl)-2-(pyridin-4-yl)benzo[d]imidazo[2,1- |
| | b]thiazole-7-carboxamide |
| 170 | 6-methoxy-N-(piperidin-4-yl)-2-(m-tolyl)benzo[d]imidazo[2,1-b]thiazole-7- |
| | carboxamide |
| 171 | N-(2-aminoethyl)-6-methoxy-2-(m-tolyl)benzo[d]imidazo[2,1-b]thiazole-7- |
| | carboxamide |
| 172 | 2-(2-fluoro-4-(pyrrolidin-2-yl)phenyl)-N-(3-(4-fluoropiperidin-1-yl)propyl)-6- |
| | methoxybenzo[d]imidazo[2,1-b]oxazole-7-carboxamide |
| 173 | N-(3-aminopropyl)-6-methoxy-2-(m-tolyl)benzo[d]imidazo[2,1-b]thiazole-7- |
| | carboxamide |
| 174 | 2-(2-fluoro-4-(pyrrolidin-2-yl)phenyl)-6-methoxy-N,N- |
| | dimethylbenzo[d]imidazo[2,1-b]thiazole-7-carboxamide |
| 175 | 2-(2-fluoro-4-(pyrrolidin-2-yl)phenyl)-6-methoxy-N-(1-methylpiperidin-4- |
| | yl)benzo[d]imidazo[2,1-b]thiazole-7-carboxamide |
| 176 | N-(2-(2-fluoro-4-(pyrrolidin-2-yl)phenyl)-6-methoxybenzo[d]imidazo[2,1- |
| | b]thiazol-7-yl)acetamide |
| 177 | N-(3-(4-fluoropiperidin-1-yl)propyl)-7-methoxy-2-(m-tolyl)benzo[4,5]thiazolo[3,2- |
| | b][1,2,4]triazole-6-carboxamide |
| 178 | 2-(2-fluoro-4-(pyrrolidin-2-yl)phenyl)-N-(3-(4-fluoropiperidin-1-yl)propyl)-7- |
| | methoxybenzo[4,5]thiazolo[3,2-b][1,2,4]triazole-6-carboxamide |
| 179 | 2-(2-fluoro-4-(methylcarbamoyl)phenyl)-N-(3-(4-fluoropiperidin-1-yl)propyl)-7- |
| | methoxybenzo[4,5]thiazolo[3,2-b][1,2,4]triazole-6-carboxamide |
| 180 | 2-(2-fluoro-4-(pyrrolidin-2-yl)phenyl)-6-hydroxybenzo[d]imidazo[2,1-b]thiazole-7- |
| | carboxamide |
| 181 | 2-(2-fluoro-4-(pyrrolidin-2-yl)phenyl)-7-methoxy-N-methylbenzo[4,5]thiazolo[3,2- |
| | bl[1,2,4]triazole-6-carboxamide |
| 182 | 2-(2-fluoro-4-(pyrrolidin-3-yl)phenyl)-N-(3-(4-fluoropiperidin-1-yl)propyl)-6- |
| | methoxybenzo[d]imidazo[2,1-b]thiazole-7-carboxamide |
| 183 | 2-(2-fluoro-4-(pyrrolidin-3-yl)phenyl)-6-methoxybenzo[d]imidazo[2,1-b]thiazole- |
| | 7-carboxamide |
| 184 | 2-(2-fluoro-4-(pyrrolidin-3-yl)phenyl)-6-methoxy-N-methylbenzo[d]imidazo[2,1- |
| | b]thiazole-7-carboxamide |
| 185 | 2-(2-fluoro-4-(pyrrolidin-3-yl)phenyl)-6-methoxy-N-(1-methylpiperidin-4- |
| | yl)benzo[d]imidazo[2,1-b]thiazole-7-carboxamide |
| 186 | 2-(2-fluoro-4-(piperidin-4-yl)phenyl)-N-(3-(4-fluoropiperidin-1-yl)propyl)-6- |
| | methoxybenzo[d]imidazo[2,1-b]thiazole-7-carboxamide |
| 187 | 2-(2,3-difluoro-4-(pyrrolidin-2-yl)phenyl)-N-(3-(4-fluoropiperidin-1-yl)propyl)-6- |
| | methoxybenzo[d]imidazo[2,1-b]thiazole-7-carboxamide |
| 188 | 6-chloro-2-(2-fluoro-4-(pyrrolidin-2-yl)phenyl)-N-(3-(4-fluoropiperidin-1- |
| | yl)propyl)benzo[d]imidazo[2,1-b]thiazole-7-carboxamide |
| 189 | (R)-2-(2-fluoro-4-(pyrrolidin-2-yl)phenyl)-6-methoxy-N-(1-methylpiperidin-4- |
| | yl)benzo[d]imidazo[2,1-b]thiazole-7-carboxamide |
| 190 | (S)-2-(2-fluoro-4-(pyrrolidin-2-yl)phenyl)-6-methoxy-N-(1-methylpiperidin-4- |
| | yl)benzo[d]imidazo[2,1-b]thiazole-7-carboxamide |
| 191 | 2-(2-fluoro-4-(4-hydroxypiperidin-4-yl)phenyl)-N-(3-(4-fluoropiperidin-1- |
| | yl)propyl)-6-methoxybenzo[d]imidazo[2,1-b]thiazole-7-carboxamide |
| 192 | 2-(2-fluoro-4-(piperidin-3-yl)phenyl)-N-(3-(4-fluoropiperidin-1-yl)propyl)-6- |
| | methoxybenzo[d]imidazo[2,1-b]thiazole-7-carboxamide |
| 195 | 2-(2-fluoro-4-(morpholin-3-yl)phenyl)-N-(3-(4-fluoropiperidin-1-yl)propyl)-6- |
| | methoxybenzo[d]imidazo[2,1-b]thiazole-7-carboxamide |
| 196 | 2-(2-fluoro-4-(pyrrolidin-2-yl)phenyl)-N-(3-(4-fluoropiperidin-1- |
| | yl)propyl)imidazo[1,2-a]quinoline-7-carboxamide |
| 197 | 2-(2-fluoro-4-(3-hydroxypyrrolidin-3-yl)phenyl)-N-(3-(4-fluoropiperidin-1- |
| | yl)propyl)-6-methoxybenzo[d]imidazo[2,1-b]thiazole-7-carboxamide |
| 198 | 6-ethoxy-2-(2-fluoro-4-(pyrrolidin-2-yl)phenyl)-N-(3-(4-fluoropiperidin-1- |
| | yl)propyl)benzo[d]imidazo[2,1-b]thiazole-7-carboxamide |
| 199 | 2-(2-fluoro-4-(pyrrolidin-2-yl)phenyl)-N-(3-(4-fluoropiperidin-1-yl)propyl)-4H- |
| | benzo[b]imidazo[1,2-d][1,4]oxazine-7-carboxamide |
| 200S | 2-(2-fluoro-4-((2S,4S)-4-hydroxypyrrolidin-2-yl)phenyl)-N-(3-(4-fluoropiperidin-1- |
| | yl)propyl)-6-methoxybenzo[d]imidazo[2,1-b]thiazole-7-carboxamide |
| 200R | 2-(2-fluoro-4-((2S,4R)-4-hydroxypyrrolidin-2-yl)phenyl)-N-(3-(4-fluoropiperidin-]- |
| | yl)propyl)-6-methoxybenzo[d]imidazo[2,1-b]thiazole-7-carboxamide |
| 201S | 2-(2-fluoro-4-((2S,4S)-4-hydroxypyrrolidin-2-yl)phenyl)-6- |
| | methoxybenzo[d]imidazo[2,1-b]thiazole-7-carboxamide |
| 201R | 2-(2-fluoro-4-((2S,4R)-4-hydroxypyrrolidin-2-yl)phenyl)-6- |
| | methoxybenzo[d]imidazo[2,1-b]thiazole-7-carboxamide |
| 202S | 2-(2-fluoro-4-((2S,4S)-4-hydroxypyrrolidin-2-yl)phenyl)-6-methoxy-N- |
| | methylbenzo[d]imidazo[2,1-b]thiazole-7-carboxamide |
| 202R | 2-(2-fluoro-4-((2S,4R)-4-hydroxypyrrolidin-2-yl)phenyl)-6-methoxy-N- |
| | methylbenzo[d]imidazo[2,1-b]thiazole-7-carboxamide |
| 203S | 2-(2-fluoro-4-((2S,4S)-4-hydroxypyrrolidin-2-yl)phenyl)-6-methoxy-N-(1- |
| | methylpiperidin-4-yl)benzo[d]imidazo[2,1-b]thiazole-7-carboxamide |
| 203R | 2-(2-fluoro-4-((2S,4R)-4-hydroxypyrrolidin-2-yl)phenyl)-6-methoxy-N-(1- |
| | methylpiperidin-4-yl)benzo[d]imidazo[2,1-b]thiazole-7-carboxamide |
| 204 | 2-(2-fluoro-4-(pyrrolidin-2-yl)phenyl)-N-(1-methylpiperidin-4-yl)-4H- |
| | benzo[b]imidazo[1,2-d][1,4]oxazine-7-carboxamide |
| [symbol] | [symbol] |
| | [symbol] |

12. The compound according to claim 1, represented by the structure of formula I(l): 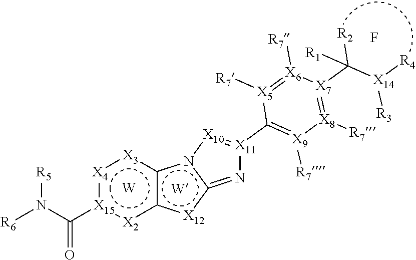
wherein
Ring F is absent or is a substituted or unsubstituted, saturated or unsaturated, 3-8 membered heterocyclic or carbocyclic ring (e.g., pyrrolidine, 1-methylpyrrolidine, pyrrolidine-3-ol, pyrrolidin-2-one, pyridine, piperidine, piperidine-4-ol, tetrahydrofurane, morpholine, tetrahydrothiophene, cyclopropyl, oxetane, oxetan-3-ol, imidazole, pyrimidine, triazole, oxadiazole, pyrazole);
R₁ and R₂ are each independently H, F, Cl, Br, I, OH, SH, or CF₃, substituted or unsubstituted C₁-C₅ alkyl, C₁-C₅ linear or branched, or C₃-C₈ cyclic haloalkyl, substituted or unsubstituted C₁-C₅ linear or branched, or C₃-C₈ cyclic alkoxy;
or R₁ and R₂ are joined to form a 3-8 membered carbocyclic or heterocyclic ring (e.g., cyclopropyl, oxetane);
or R₂ and R₄ are joined to form Ring F as defined above (e.g., pyrrolidine, pyrrolidin-2-one, piperidine, morpholine, pyridine, pyrimidine, triazole, oxadiazole, pyrazole, tetrahydrofurane), wherein if Ring F is aromatic, then R₁ and/or R₃ are absent;
R₃ and R₄ are each independently H, Me, substituted or unsubstituted C₁-C₅ alkyl (e.g., methoxyethylene, methylaminoethyl, aminoethyl), —R₈—O—R₁₀ (e.g., (CH₂)₂O—CH₃), R₈—N(R₁₀)(R₁₁) (e.g., (CH₂)₂—NH(CH₃)), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀;
or R₃ and R₄ are joined to form a 3-8 membered heterocyclic ring (e.g., pyrrolidine, pyrrolidone, 2-oxopyrrolidine, piperidine, morpholine, piperazine, imidazole); and
X₁₄ is S, O, N or CH, wherein if X₁₄ is CH then Ring F is not absent and if X₁₄ is S or O then R₃ is absent.

13. The compound of claim 12, represented by the structure of formula I(m): 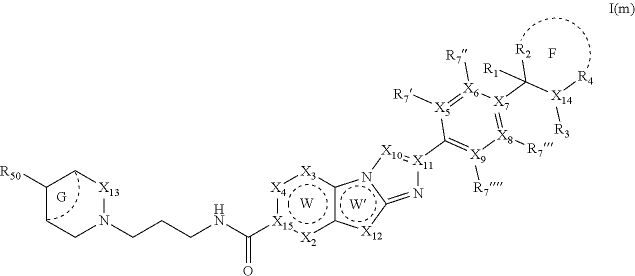
wherein
X₁₃ is CH₂, CH(R) (e.g., CH—CH₃), C(R)₂, or C═O;
Ring G is absent or is a substituted or unsubstituted 3-8 membered carbocyclic or heterocyclic ring (e.g., cyclobutane, cyclopentane, cyclohexane);
R₅₀ is H, R₂₀, F, Cl, Br, I, OH, SH, N(R)₂, NH(R₁₀), N(R₁₀)(R₁₁), CF₃, CN, NO₂, C₁-C₅ linear or branched, substituted or unsubstituted alkyl C₁-C₅ linear or branched alkoxy, C₁-C₅ linear or branched haloalkyl, —R₈-R₁₀ (e.g., (CH₂)₂O—CH₃), substituted or unsubstituted aryl (e.g., phenyl), substituted or unsubstituted heteroaryl (e.g., pyridine (2, 3, and 4-pyridine).

14. The compound of claim 12, wherein X₁₄ is O or N.

15. The compound of claim 12, wherein R₁ is H, or wherein R₁ and R₂ are joined to form a 3-8 membered carbocyclic or heterocyclic ring (e.g., cyclopropyl, oxetane, pyrrolidine).

16. The compound of claim 12, wherein R₃ is H, substituted or unsubstituted C₁-C₅ alkyl or absent.

17. The compound of claim 12, wherein R₂ and R₄ are joined to form Ring F (preferably pyrrolidine, piperidine, tetrahydrofurane, pyrrolidin-2-one, pyrrolidin-3-ol, morpholine).

18. (canceled)

19. The compound of claim 1, wherein the compound is a substantially pure single stereoisomer.

20. The compound according to claim 1, wherein the compound is a c-MYC mRNA translation modulator, a c-MYC mRNA transcription regulator, a c-MYC inhibitor or any combination thereof.

21. A pharmaceutical composition comprising the compound of claim 1 and a pharmaceutically acceptable carrier.

22. A method of treating, suppressing, reducing the severity, reducing the risk of developing or inhibiting cancer in a subject, comprising administering a therapeutically effective amount of a compound according to claim 1 to a subject, thereby treating, suppressing, reducing the severity, reducing the risk of developing or inhibiting cancer in said subject.

23. The method of claim 22,
wherein the cancer is selected from the list of: breast cancer, ovarian carcinoma, acute myeloid leukemia, chronic myelogenous leukemia, Hodgkin's and Burkitt's lymphoma, diffuse large Bcell lymphoma, prostate cancer, colon cancer, gastric cancer, primary central nervous system lymphoma, glioblastoma, medulloblastoma, melanoma, non-small cell lung carcinoma, germinal center-derived lymphomas, esophageal squamous cell carcinoma, osteosarcoma, bladder cancer, pancreatic cancer, lung adenocarcinoma, BRAF V600E thyroid cancer, choroid plexus carcinoma, colitis-associated cancer, epithelial ovarian cancer, colorectal cancer, pancreatic cancer and uterine cancer;
wherein the cancer is early cancer, advanced cancer, invasive cancer, metastatic cancer, drug resistant cancer or any combination thereof;
wherein the subject has been previously treated with chemotherapy, immunotherapy, radiotherapy, biological therapy, surgical intervention, or any combination thereof;
wherein the compound is administered in combination with an anti-cancer therapy;
or any combination thereof.

24. The method of claim 23, wherein the anti-cancer therapy is chemotherapy, immunotherapy, radiotherapy, biological therapy, surgical intervention, or any combination thereof.

25. A method of suppressing, reducing or inhibiting tumor growth in a subject, comprising administering a therapeutically effective amount of a compound according to claim 1 to a subject thereby suppressing, reducing or inhibiting tumor growth in said subject.

26. A method of modulating c-MYC mRNA translation or regulating c-MYC mRNA transcription in a cell, comprising contacting a compound according to claim 1 with a cell, thereby modulating c-MYC mRNA translation, or regulating c-MYC mRNA transcription in said cell.

27. The method of claim 26, wherein said method is carried out
(a) by regulating c-MYC mRNA splicing (inclusion or exclusion of untranslated region or alternative usage of exons);
(b) by regulation of c-MYC mRNA modifications;
(c) by regulation of the interaction of RNA binding protein with c-MYC mRNA thereby changing mRNA localization;
(d) by regulating c-MYC mRNA localization in the cytoplasm;
(e) by regulating ribosomes or ribosome accessory factor to c-MYC mRNA;
(f) by reducing the amount of c-MYC protein in the cell;
or any combination thereof.
